SEQUENCE LISTING (59 sequences)

SEQ ID NO: 1 - protein, 6 aa, Artificial Sequence
  Description of Artificial Sequence Synthetic peptide
LLIEML

SEQ ID NO: 2 - DNA, 45 bp, Artificial Sequence
  Description of Artificial Sequence Synthetic primer
gctggatccgaaaacctgtacttccagggaggccccaacgtgcct

SEQ ID NO: 3 - DNA, 40 bp, Artificial Sequence
  Description of Artificial Sequence Synthetic primer
ggatccatgtcgactcaagtctgcttggcttgcagcattt

SEQ ID NO: 4 - DNA, 51 bp, Artificial Sequence
  Description of Artificial Sequence Synthetic primer
gctggatccgaaaacctgtacttccagggttctccagcaagcatcccacat

SEQ ID NO: 5 - DNA, 34 bp, Artificial Sequence
  Description of Artificial Sequence Synthetic primer
gttcttgtcgacttatgctcttttggcatgcaac

SEQ ID NO: 6 - DNA, 29 bp, Artificial Sequence
  Description of Artificial Sequence Synthetic primer
cgcatcttgggctctctgcaggagcccac

SEQ ID NO: 7 - DNA, 28 bp, Artificial Sequence
  Description of Artificial Sequence Synthetic primer
cactacccgcactccggggacaaattcc

SEQ ID NO: 8 - DNA, 30 bp, Artificial Sequence
  Description of Artificial Sequence Synthetic primer
gttcttcatatgggaggccccaacgtgcct

SEQ ID NO: 9 - DNA, 33 bp, Artificial Sequence
  Description of Artificial Sequence Synthetic primer
gttcttcatatgtctccagcaagcatcccacat

SEQ ID NO: 10 - DNA, 27 bp, Artificial Sequence
  Description of Artificial Sequence Synthetic primer
cgggcccgcatcaagggctgcctgcag

SEQ ID NO: 11 - DNA, 30 bp, Artificial Sequence
  Description of Artificial Sequence Synthetic primer
ctcctgtgcagaatgttcgaccagaccttc

SEQ ID NO: 12 - DNA, 27 bp, Artificial Sequence
  Description of Artificial Sequence Synthetic primer
ggcaggaggtggcactgaccacagtgg

SEQ ID NO: 13 - DNA, 37 bp, Artificial Sequence
  Description of Artificial Sequence Synthetic primer
cacagtggccacccaggcggagtcgctgctgcacagc

SEQ ID NO: 14 - DNA, 30 bp, Artificial Sequence
  Description of Artificial Sequence Synthetic primer
gcgggctcgctgttccacagcctggtgttg

SEQ ID NO: 15 - DNA, 31 bp, Artificial Sequence
  Description of Artificial Sequence Synthetic primer
cctgagcatgcagttcaaggagtacctgtac

SEQ ID NO: 16 - DNA, 29 bp, Artificial Sequence
  Description of Artificial Sequence Synthetic primer
gcaggccaaggagatgctgtaccacaagc

SEQ ID NO: 17 - DNA, 28 bp, Artificial Sequence
  Description of Artificial Sequence Synthetic primer
gtacctgtaccacatgcacctgggcaac

SEQ ID NO: 18 - DNA, 40 bp, Artificial Sequence
  Description of Artificial Sequence Synthetic primer
gcaggccaaggagatgctgtaccacatgcacctgggcaac

SEQ ID NO: 19 - DNA, 40 bp, Artificial Sequence
  Description of Artificial Sequence Synthetic primer
gcaggccaaggagttcctgtaccacgcgcacctgggcaac

SEQ ID NO: 20 - DNA, 35 bp, Artificial Sequence
  Description of Artificial Sequence Synthetic primer
gcaacaacctgctcatcaagatgctgcaagccaag

SEQ ID NO: 21 - DNA, 31 bp, Artificial Sequence
  Description of Artificial Sequence Synthetic primer
gtccaggctaaaatcaaggcctatttgcagc

SEQ ID NO: 22 - DNA, 32 bp, Artificial Sequence
  Description of Artificial Sequence Synthetic primer
gagcacctttgggtacatgtgcaaaatggcag

SEQ ID NO: 23 - DNA, 33 bp, Artificial Sequence
  Description of Artificial Sequence Synthetic primer
cttatgtgcaaaatgttcgatcaaactctcttc

SEQ ID NO: 24 - DNA, 33 bp, Artificial Sequence
  Description of Artificial Sequence Synthetic primer
cttatgtgcaaaatgatggatcaaactctcttc

SEQ ID NO: 25 - DNA, 34 bp, Artificial Sequence
  Description of Artificial Sequence Synthetic primer
ctgggcaacaagtggcatattccataatagcatc

SEQ ID NO: 26 - DNA, 32 bp, Artificial Sequence
  Description of Artificial Sequence Synthetic primer
caagtggactattcctacatagcatcacaagc

SEQ ID NO: 27 - DNA, 29 bp, Artificial Sequence
  Description of Artificial Sequence Synthetic primer
gccggagccaccttcaacaacctcatgag

SEQ ID NO: 28 - DNA, 32 bp, Artificial Sequence
  Description of Artificial Sequence Synthetic primer
ccatcagtatgcagttcgaagaatacctctac

SEQ ID NO: 29 - DNA, 32 bp, Artificial Sequence
  Description of Artificial Sequence Synthetic primer
ccatcagtatgcagatggaagaatacctctac

SEQ ID NO: 30 - DNA, 39 bp, Artificial Sequence
  Description of Artificial Sequence Synthetic primer
gcaggctgaagaattcctctactacgcgcacctgaacgg

SEQ ID NO: 31 - DNA, 39 bp, Artificial Sequence
  Description of Artificial Sequence Synthetic primer
ctataataaccttctcattaagatgttgcatgccaaaag

SEQ ID NO: 32 - protein, 16 aa, Artificial Sequence
  Description of Artificial Sequence Synthetic peptide
VDLNDIFEAQKIEWHR

SEQ ID NO: 33 - protein, 20 aa, Artificial Sequence
  Description of Artificial Sequence Synthetic peptide
MDGAPDSALRQLLSQKPMEP

SEQ ID NO: 34 - DNA, 1011 bp, Artificial Sequence
  Description of Artificial Sequence Synthetic construct
taatacgactcactataggggaattgtgagcggataacaattcccctctagaaataattttgtttaactttaagaaggagatataccatgaaaaaaggtcaccaccatcaccatcacggatccgaaaacctgtacttccagggaggccccaacgtgcctgagctcatcctgcagctgctgcagctggagccggatgaggaccaggtgcgggcccgcatcttgggctctctgcaggagcccaccaaaagccgccccgaccagccggcggccttcggcctcctgtgcagaatggccgaccagaccttcatctccatcgtggactgggcacgcaggtgcatggtcttcaaggagctggaggtggccgaccagatgacgctgctgcagaactgctggagcgagctgctggtgttcgaccacatctaccgccaggtccagcacggcaaggagggcagcatcctgctggtcaccgggcaggaggtggagctgaccacagtggccacccaggcgggctcgctgctgcacagcctggtgttgcgggcgcaggagctggtgctgcagctgcttgcgctgcagctggaccggcaggagtttgtctgcctcaagttcatcatcctcttcagcctggatttgaagttcctgaataaccacatcctggtgaaagacgctcaggagaaggccaacgccgccctgcttgactacaccctgtgccactacccgcactccggggacaaattccagcagctactgctgtgcctggtggaggtgcgggccctgagcatgcaggccaaggagtacctgtaccacaagcacctgggcaacgagatgccccgcaacaacctgctcatcgaaatgctgcaagccaagcagacttgagtcgaccaccaccaccaccaccactgagatccggctggccctactggccgaaaggaattcgaggccagcagggccaccgctgagcaataactagcataaccccttggggcctctaaacgggtcttgaggggttttttg

SEQ ID NO: 35 - protein, 261 aa, Artificial Sequence
  Description of Artificial Sequence Synthetic construct
MKKGHHHHHHGSENLYFQGGPNVPELILQLLQLEPDEDQVRARILGSLQEPTKSRPDQPAAFGLLCRMADQTFISIVDWARRCMVFKELEVADQMTLLQNCWSELLVFDHIYRQVQHGKEGSILLVTGQEVELTTVATQAGSLLHSLVLRAQELVLQLLALQLDRQEFVCLKFIILFSLDLKFLNNHILVKDAQEKANAALLDYTLCHYPHSGDKFQQLLLCLVEVRALSMQAKEYLYHKHLGNEMPRNNLLIEMLQAKQT

SEQ ID NO: 36 - DNA, 1020 bp, Artificial Sequence
  Description of Artificial Sequence Synthetic construct
taatacgactcactataggggaattgtgagcggataacaattcccctctagaaataattttgtttaactttaagaaggagatataccatgaaaaaaggtcaccaccatcaccatcacggatccgaaaacctgtacttccagggttctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagtcgaccaccaccaccaccaccactgagatccggctggccctactggccgaaaggaattcgaggccagcagggccaccgctgagcaataactagcataaccccttggggcctctaaacgggtcttgaggggttttttg

SEQ ID NO: 37 - protein, 264 aa, Artificial Sequence
  Description of Artificial Sequence Synthetic construct
MKKGHHHHHHGSENLYFQGSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 38 - protein, 18 aa, Artificial Sequence
  Description of Artificial Sequence Synthetic peptide
MKKGHHHHHHGSENLYFQ

SEQ ID NO: 39 - protein, 25 aa, Artificial Sequence
  Description of Artificial Sequence Synthetic peptide
CPSSHSSLTERHKILHRLLQEGSPS

SEQ ID NO: 40 - protein, 13 aa, Artificial Sequence
  Description of Artificial Sequence Synthetic peptide
KENALLRYLLDKD

SEQ ID NO: 41 - protein, 13 aa, Artificial Sequence
  Description of Artificial Sequence Synthetic peptide
KENALLRYLLDKD

SEQ ID NO: 42 - DNA, 3119 bp, Homo sapiens
ggaggacggacggacagggccagcctgctgtccggctgccgcccgccgtggtgtgagggggtttctgcgcacccacagtcgccaccgtcccacctgggctgccggagcctccccctggacccctggtgcccactgccaccctcatccggtgtgagagcgctgcttccgcttcgcggacgccgcgggcatggactattcgtacgacgaggacctggacgagctgtgccccgtgtgcggggacaaggtgtccggctaccactacggactgctcacgtgtgagagctgcaagggcttcttcaagcgcacggtgcagaacaacaagcactacacgtgcaccgagagccagagctgcaagatcgacaagacgcagcgcaagcgctgtcccttctgccgcttccagaaatgcctgacggtggggatgcgcctggaagccgtgcgcgctgaccgtatgaggggtggccggaacaagtttgggccgatgtacaagcgggaccgggccctgaaacagcagaagaaggcacagattcgggccaatggcttcaagctggagacagggcccccgatgggggtgcccccgccgccccctcccgcaccggactacgtgctgcctcccagcctgcatgggcctgagcccaagggcctggccgccggtccacctgctgggccactgggcgactttggggccccagcactgcccatggccgtgcccggtgcccacgggccactggctggctacctctaccctgcctttcctggccgtgccatcaagtctgagtacccggagccttatgccagccccccacagcctgggctgccgtacggctacccagagcccttctctggagggcccaacgtgcctgagctcatcctgcagctgctgcagctggagccggatgaggaccaggtgcgggcccgcatcttgggctgcctgcaggagcccaccaaaagccgccccgaccagccggcggccttcggcctcctgtgcagaatggccgaccagaccttcatctccatcgtggactgggcacgcaggtgcatggtcttcaaggagctggaggtggccgaccagatgacgctgctgcagaactgctggagcgagctgctggtgttcgaccacatctaccgccaggtccagcacggcaaggagggcagcatcctgctggtcaccgggcaggaggtggagctgaccacagtggccacccaggcgggctcgctgctgcacagcctggtgttgcgggcgcaggagctggtgctgcagctgcttgcgctgcagctggaccggcaggagtttgtctgcctcaagttcatcatcctcttcagcctggatttgaagttcctgaataaccacatcctggtgaaagacgctcaggagaaggccaacgccgccctgcttgactacaccctgtgccactacccgcactgcggggacaaattccagcagctgctgctgtgcctggtggaggtgcgggccctgagcatgcaggccaaggagtacctgtaccacaagcacctgggcaacgagatgccccgcaacaacctgctcatcgaaatgctgcaagccaagcagacttgagcctgggccgggggcggggccgggactgggggcgggactgggggcggggcctgggcggggccgcagccacaccgctggctctgcatggttcattttctgatgcccaccgaggagccccagccccgtcccagaggccgctgcccctgagttctgacactgtgtgtttgggaagtgggtgaggctgggcagggcctggcggaggtggagtggccactggcacttgcctgctgcttggagtgccccaaggaggtggctgttaaccacccgccccgccccctccctgctcccagctctctctcctggagtctgaagcctgcaggtccggggaggaggttcgggattccctggtgggcctcgacgtcccttggatcagaggtcatcccttcctcctctcctggaaacagacagggagaagttgagcaggtatcaactaggggaggagagagggtctccagtgttccccccatagagaccaggagggagagcctctgttttgtaaactaaggataaccgagtttgctaaattgagaggggctattgggccctagaggacactaggagactggttaggacaaaaagaccttctccctagcccttctaccccacctgacctctgcaagagggggcattgatacatcatcgggaaaaaactttgctccaggcatcactgattccctctcccacccaaggagaacgtttggtacaatcgacatcctagccccacccagaggtggccctcccaggctggtatttatctgcaaggttgtagtcaagaggtttttctccccgctttttgtttttaagcttctagacactccttgaaatgtgtgtgtgatggagggaaggggacagatttgaggactgaagctggggcttggggattgccactaagtacagctgatggtttctccccggacactcgcctactaagtacccttggggtggtgctgggtcattacttctgagccccagccccaatccagagaagcgctgttgcccgccctccacccactaggtgaacagcaggatgccctgttgggggcttcaggtctctgtgggtgggaatgcaagtgaacttgggagggggcacgggcctgtagatcagggatagcgctgttgatcccctctctgtggctccaacccgttgggtcccttgctgcaaacccatgaagctggccctcagctccctgaccccctgtcctaggtcatgaaggacactctgcagggtgaagcaccagggagaggcctcggctgtctcctgtccccggcggggtgcctgctgtccgtcccgctttcatgttactgttgcagcttgtgctgagcctgcccagttggaggagactgggcacccctgcctcctgcctcccgcctcccgccaccctgtctcagtacctcccccccccgccccctgaaacatgtgcccctgccaaggccggagacccacagccctgaaacgagaagtgcccttaaggatcaccccagcccccacagccctggaataaatttcgcaattagtttccaaaaaaaaaaaaaaaaaaaaaaaaaa

SEQ ID NO: 43 - protein, 461 aa, Homo sapiens
MDYSYDEDLDELCPVCGDKVSGYHYGLLTCESCKGFFKRTVQNNKHYTCTESQSCKIDKTQRKRCPFCRFQKCLTVGMRLEAVRADRMRGGRNKFGPMYKRDRALKQQKKAQIRANGFKLETGPPMGVPPPPPPAPDYVLPPSLHGPEPKGLAAGPPAGPLGDFGAPALPMAVPGAHGPLAGYLYPAFPGRAIKSEYPEPYASPPQPGLPYGYPEPFSGGPNVPELILQLLQLEPDEDQVRARILGCLQEPTKSRPDQPAAFGLLCRMADQTFISIVDWARRCMVFKELEVADQMTLLQNCWSELLVFDHIYRQVQHGKEGSILLVTGQEVELTTVATQAGSLLHSLVLRAQELVLQLLALQLDRQEFVCLKFIILFSLDLKFLNNHILVKDAQEKANAALLDYTLCHYPHCGDKFQQLLLCLVEVRALSMQAKEYLYHKHLGNEMPRNNLLIEMLQAKQT

SEQ ID NO: 44 - DNA, 4916 bp, Homo sapiens
aaaaagtacagagtccagggaaagacttgcttgtaactttatgaattctggatttttttttttcctttgctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattgtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatacttaatagcaaataaatgatgtatcagggtatttgtattgcaaactgtgaatcaaaggcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcgggggatttgagcttacaggattcctccatggtaaagctgaactgaaacaattctcaagaatgcatcagctgtacctacaatagcccctccctcttcctttgaaggccccagcacctctgccctgtggtcaccgaatctgtactaaggacctgtgttcagccacacccagtggtagctccaccaaatcatgaacagcctaattttgagtgtctgtgtcttagacctgcaaacagctaataggaaattctattaatatgttagcttgccattttaaatatgttctgagggttgttttgtctcgtgttcatgatgttaagaaaatgcaggcagtatccctcatcttatgtaagtgtgaattaatattaagggaaatgactacaaactttcaaagcaaatgctccatagctaaagcaacttagaccttatttctgctactgttgctgaaatgtggctttggcattgttggatttcataaaaaatttctggcaggaagtcttgttagtatacatcagtctttttcatcatccaagtttgtagttcatttaaaaatacaacattaaacacattttgctaggatgtcaaatagtcacagttctaagtagttggaaacaaaattgacgcatgttaatctatgcaaagagaaaggaaaggatgaggtgatgtattgactcaaggttcattcttgctgcaattgaacatcctcaagagttgggatggaaatggtgatttttacatgtgtcctggaaagatattaaagtaattcaaatcttccccaaaggggaaaggaagagagtgatactgacctttttaagtcatagaccaaagtctgctgtagaacaaatatgggaggacaaagaatcgcaaattcttcaaatgactattatcagtattattaacatgcgatgccacaggtatgaaagtcttgccttatttcacaattttaaaaggtagctgtgcagatgtggatcaacatttgtttaaaataaagtattaatactttaaagtcaaataagatatagtgtttacattctttaggtcctgaggggcagggggatctgtgatataacaaaatagcaaaagcggtaatttccttaatgttatttttctgattggtaattatttttaacagtacttaattattctatgtcgtgagacactaaaatcaaaaacgggaatctcatttagactttaatttttttgagattatcggcggcacaatcactttgtagaaactgtaaaaaataaaagtatctcctagtcccttaattttttcataaatatttctggcttttgagtagtgtatttatattgtatatcatactttcaactgtagacaattatgatgctaatttattgtttcttggtttcacctttgtataagatatagccaagactgaagaaaccaaatatatgtgtttactgtagcatgtcttcaaattagtggaacttagttcagggacatagaagagtcttaatgaattaaaatcattcacttgattaaatgtctgtaaatcttcatcattcctactgtagtttatttaatatctattgtaaattatgtgacttgtagcttcctctggttttcaagtaaactcaacaaggtggagtcttacctggttttcctttccaagcattgtaaattgtataccaaagatattagttattacttctgtgtgtacaaagaggattattttattatgtttattaatcacctctaatactcatccacatgaagggtacacattaggtaagctgggcgttgactcatgcgcagtctcagtcacccgtgttatcttcgtggctcaaaggacaatgcaaaatcgccgatcagagctcatacccaaagcattacagagaacagcagcatcattgccctccccagctgaaaaacaagttggctagaagatacatggagaggaatggtgtggtcaacagttaatgaaacggttctatcatgcatgtgtaatgtggatggagacaattataagatttgactataactatttggagggtctttaacattgccaaaaaaacaaatatgttgatttttattttattttattttttattttaagaggcgggatcttgatctcacatgttgcccaggctggccttgaactcctgggctcaagcattcctcctgcctcagcctcccccatagctgggactaggggtgcatgccagcatacctggctacgttgactcttaaaatctatgttctcttattttaaagatacagtgctccccactgaaaattaaacctaaaaaatgtcacatattggtatgttgttaacctggtagattaaatcatgagaatgattagaaagacgggcaacacagcgggttacatccacactgctgatcacaccaacgacaggagctgataagcaagaaagcgtcacagccagcgtctgttcacccaaggttgacaagtgaagtttctctaatgttgattgttagccgatttgtaacctggcatttacttagcaactgccttatcaattacaggatttgccggtaaaagcagactcaaatataaaggtttttggcttaacttggtttattatagttgctctatgtttgtaaacagacaatctctaatgtctgattatttgtatcacagatctgcagctgccttggacttgaatccatgcaatgtttagagtgtgaagtcagttacttgttgatgttttcttactgtatcaatgaaatacatattgtcatgtcagttcttgccaggaacttctcaacaaaatggaattttttttttcagtatttcaataaatattgatatgcccagcctgataatttttaaaaaaaaaaaa

SEQ ID NO: 45 - protein, 495 aa, Homo sapiens
MSSNSDTGDLQESLKHGLTPIVSQFKMVNYSYDEDLEELCPVCGDKVSGYHYGLLTCESCKGFFKRTVQNNKRYTCIENQNCQIDKTQRKRCPYCRFQKCLSVGMKLEAVRADRMRGGRNKFGPMYKRDRALKQQKKALIRANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 46 - DNA, 5054 bp, Homo sapiens
aaaaagtacagagtccagggaaagacttgcttgtaactttatgaattctggatttttttttttcctttgctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattggtgctgggcttccggaccgacacggatcccccatccccgcccgcggtcgccttgtcatgctgcccaaagtggagacggaagccctgggactggctcgatcgcatggggaacagggccagatgccggaaaacatgcaagtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatacttaatagcaaataaatgatgtatcagggtatttgtattgcaaactgtgaatcaaaggcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcgggggatttgagcttacaggattcctccatggtaaagctgaactgaaacaattctcaagaatgcatcagctgtacctacaatagcccctccctcttcctttgaaggccccagcacctctgccctgtggtcaccgaatctgtactaaggacctgtgttcagccacacccagtggtagctccaccaaatcatgaacagcctaattttgagtgtctgtgtcttagacctgcaaacagctaataggaaattctattaatatgttagcttgccattttaaatatgttctgagggttgttttgtctcgtgttcatgatgttaagaaaatgcaggcagtatccctcatcttatgtaagtgtgaattaatattaagggaaatgactacaaactttcaaagcaaatgctccatagctaaagcaacttagaccttatttctgctactgttgctgaaatgtggctttggcattgttggatttcataaaaaatttctggcaggaagtcttgttagtatacatcagtctttttcatcatccaagtttgtagttcatttaaaaatacaacattaaacacattttgctaggatgtcaaatagtcacagttctaagtagttggaaacaaaattgacgcatgttaatctatgcaaagagaaaggaaaggatgaggtgatgtattgactcaaggttcattcttgctgcaattgaacatcctcaagagttgggatggaaatggtgatttttacatgtgtcctggaaagatattaaagtaattcaaatcttccccaaaggggaaaggaagagagtgatactgacctttttaagtcatagaccaaagtctgctgtagaacaaatatgggaggacaaagaatcgcaaattcttcaaatgactattatcagtattattaacatgcgatgccacaggtatgaaagtcttgccttatttcacaattttaaaaggtagctgtgcagatgtggatcaacatttgtttaaaataaagtattaatactttaaagtcaaataagatatagtgtttacattctttaggtcctgaggggcagggggatctgtgatataacaaaatagcaaaagcggtaatttccttaatgttatttttctgattggtaattatttttaacagtacttaattattctatgtcgtgagacactaaaatcaaaaacgggaatctcatttagactttaatttttttgagattatcggcggcacaatcactttgtagaaactgtaaaaaataaaagtatctcctagtcccttaattttttcataaatatttctggcttttgagtagtgtatttatattgtatatcatactttcaactgtagacaattatgatgctaatttattgtttcttggtttcacctttgtataagatatagccaagactgaagaaaccaaatatatgtgtttactgtagcatgtcttcaaattagtggaacttagttcagggacatagaagagtcttaatgaattaaaatcattcacttgattaaatgtctgtaaatcttcatcattcctactgtagtttatttaatatctattgtaaattatgtgacttgtagcttcctctggttttcaagtaaactcaacaaggtggagtcttacctggttttcctttccaagcattgtaaattgtataccaaagatattagttattacttctgtgtgtacaaagaggattattttattatgtttattaatcacctctaatactcatccacatgaagggtacacattaggtaagctgggcgttgactcatgcgcagtctcagtcacccgtgttatcttcgtggctcaaaggacaatgcaaaatcgccgatcagagctcatacccaaagcattacagagaacagcagcatcattgccctccccagctgaaaaacaagttggctagaagatacatggagaggaatggtgtggtcaacagttaatgaaacggttctatcatgcatgtgtaatgtggatggagacaattataagatttgactataactatttggagggtctttaacattgccaaaaaaacaaatatgttgatttttattttattttattttttattttaagaggcgggatcttgatctcacatgttgcccaggctggccttgaactcctgggctcaagcattcctcctgcctcagcctcccccatagctgggactaggggtgcatgccagcatacctggctacgttgactcttaaaatctatgttctcttattttaaagatacagtgctccccactgaaaattaaacctaaaaaatgtcacatattggtatgttgttaacctggtagattaaatcatgagaatgattagaaagacgggcaacacagcgggttacatccacactgctgatcacaccaacgacaggagctgataagcaagaaagcgtcacagccagcgtctgttcacccaaggttgacaagtgaagtttctctaatgttgattgttagccgatttgtaacctggcatttacttagcaactgccttatcaattacaggatttgccggtaaaagcagactcaaatataaaggtttttggcttaacttggtttattatagttgctctatgtttgtaaacagacaatctctaatgtctgattatttgtatcacagatctgcagctgccttggacttgaatccatgcaatgtttagagtgtgaagtcagttacttgttgatgttttcttactgtatcaatgaaatacatattgtcatgtcagttcttgccaggaacttctcaacaaaatggaattttttttttcagtatttcaataaatattgatatgcccagcctgataatttttaaaaaaaaaaaa

SEQ ID NO: 47 - protein, 541 aa, Homo sapiens
MSSNSDTGDLQESLKHGLTPIGAGLPDRHGSPIPARGRLVMLPKVETEALGLARSHGEQGQMPENMQVSQFKMVNYSYDEDLEELCPVCGDKVSGYHYGLLTCESCKGFFKRTVQNNKRYTCIENQNCQIDKTQRKRCPYCRFQKCLSVGMKLEAVRADRMRGGRNKFGPMYKRDRALKQQKKALIRANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 48 - DNA, 3027 bp, Mus musculus
tgttttttccccctttttcttaactttcactaaggaaatgagggttactgtagtctgaggtttccttcccaaagtcacaaaatatgacaagctgcaatctttctcacattcaatgatttctgctgtaagccaaaggactgccaataatttcgctaagaatgtctgctagtttggatactggagattttcaagaatttcttaagcatggacttacagctattgcgtctgcaccagggtcagagactcgccactcccccaaacgtgaggaacaactccgggaaaaacgtgctgggcttccggaccgacaccgacgccccattcccgcccgcagccgccttgtcatgctgcccaaagtggagacggaagccccaggactggtccgatcgcatggggaacaggggcagatgccagaaaacatgcaagtgtctcaatttaaaatggtgaattactcctatgatgaagatctggaagagctatgtcctgtgtgtggcgataaagtgtctgggtaccattacggtctcctcacgtgcgaaagctgcaagggtttttttaagcgaactgtccaaaaccaaaaaaggtacacgtgcatagagaaccagaattgccaaattgacaaaacgcagagaaaacgatgtccctactgtcgattcaaaaaatgtatcgatgttgggatgaagctggaagccgtaagagccgaccgcatgcgagggggcagaaataagtttgggccaatgtacaagagagacagggctttgaagcagcagaagaaagccctcattcgagccaatggacttaagctggaagccatgtctcaggtgatccaagcaatgccctcagacctgacctctgcaattcagaacattcattccgcctccaaaggcctacctctgagccatgtagccttgcctccgacagactatgacagaagtccctttgtcacatctcccattagcatgacaatgccacctcacagcagcctgcatggttaccaaccctatggtcactttcctagtcgggccatcaagtctgagtacccagacccctactccagctcacctgagtcaatgatgggttactcctacatggatggttaccagacaaactccccggccagcatcccacacctgatactggaacttttgaagtgtgaaccagatgagcctcaagttcaagcgaagatcatggcttacctccagcaagagcagagtaaccgaaacaggcaagaaaagctgagcgcatttgggcttttatgcaaaatggcggaccagaccctgttctccattgttgagtgggccaggagtagtatcttcttcagggaactgaaggttgatgaccaaatgaagctgcttcaaaactgctggagtgagctcttgattctcgatcacatttaccgacaagtggcgcatgggaaggaagggacaatcttcctggttactggagaacacgtggactactccaccatcatctcacacacagaagtcgcgttcaacaacctcctgagtctcgcacaggagctggtggtgaggctccgttcccttcagttcgatcagcgggagtttgtatgtctcaagttcctggtgctgttcagctcagatgtgaagaacctggagaacctgcagctggtggaaggtgtccaagagcaggtgaatgccgccctgctggactacacggtttgcaactacccacaacagactgagaaattcggacagctacttcttcggctacccgagatccgggcaatcagcaagcaggcagaagactacctgtactataagcacgtgaacggggatgtgccctataataacctcctcattgagatgctgcatgccaaaagagcctaagtccccacccctggaagcttgctctaggaacacagactggaaggagaagaggaggacgatgacagaaacacaatactctgaactgctccaagcaatgctaattataaacttggtttaaagacactgaattttaaaagcataataattaaatacctaatagcaaataaatgatatatcagggtatttgtactgcaaactgtgaatcaaaggctgtatgaatcaaaggattcatatgaaagacattgtaatggggtggattgaacttacagatggagaccaataccacagcagaataaaaatggacagaacaatccttgtatatttaaactaatctgctattaagaaattcagaagttgatctctgttattaattggatttgtcctgaattactccgtggtgacgctgaacaactcaagaatacatgggctgtgcttggcagcccctccccatccctcccaccaccaccacccccacccccacaaggccctataccttctgacctgtgagccctgaagctattttaaggacttctgttcagccatacccagtagtagctccactaaaccatgatttctggatgtctgtgtcttagacctgccaacagctaataagaacaatgtataaatatgtcagcttgcattttaaatatgtgctgaagtttgttttgtcgtgtgttcgtaattaaaaagaaaacgggcagtaaccctcttctatataagcattagttaatattaagggaaatcaaacaaatctaagccaatactcccaacaagcaagttagatcttacttctgctgctgttgctgaaatgtggctttggcatggttgggtttcataaaactttttggccaagaggcttgttagtatacatccatctgtttagtcatcaaggtttgtagttcacttaaaaaaaaataaaccactagacatcttttgctgaatgtcaaatagtcacagtctaagtagccaaaaagtcaaagcgtgttaaacattgccaaatgaaggaaagggtgagctgcaaaggggatggttcgaggttcattccagttgtgacccgagcgtccccaaaacctgggatgcaaagacagtgattctgcatatggcctggaaagacaggaaagccagtctcctacaaaggggaatggaagatcctggcctctaagtcatagaccaaagtctgctgtag

SEQ ID NO: 49 - protein, 241 aa, Mus musculus
PNVPELILQLLQLEPEEDQVRARIVGCLQEPAKSRSDQPAPFSLLCRMADQTFISIVDWARRCMVFKELEVADQMTLLQNCWSELLVLDHIYRQVQYGKEDSILLVTGQEVELSTVAVQAGSLLHSLVLRAQELVLQLHALQLDRQEFVCLKFLILFSLDVKFLNNHSLVKDAQEKANAALLDYTLCHYPHCGDKFQQLLLCLVEVRALSMQAKEYLYHKHLGNEMPRNNLLIEMLQAKQT

SEQ ID NO: 50 - protein, 241 aa, Macropus eugenii
PGVPELILKLLQLEPDEGQLKARILACLQEPSKGRPDRPTPFGLMCKMADQTLFSIVEWARSCVVFKELEVADQMKLLQNCWSELLVFDHIYRQIQHGKEGSILLVTGQEVDLSTVAAQAGSLLHSLVLRAQDLVQQLHSLQVDRQEFVCLKFLILFSLDVKFLENHGLAKDAQEKANSALLEYTMCHYPHCGDKFRQLLLRLAEVRSLSMQAEEYLYHKHLGGEVPCNNLLIEMLHAKRT

SEQ ID NO: 51 - protein, 242 aa, Gallus gallus
PDIPEVILKLLQLEPDEAQVKARILACLQQEQGKGRHEKLSTFGLMCKMADQTLFSIVEWARSCIFFKELEVGDQMKLLQNCWSELLVFDHVYRQLQHGKEHSVLLVTGQEVDLSAVAAQAGSILHSLVLRAQELVLHLHSLQVDRQEFVCLKFLILFSLDVKYLENHALAKDAQEKANAALLEYTVCHYPHCTDKFRQLLLRLTEVRALSMQAEEYLYHKHLSGEVPCNNLLIEMLHAKRT

SEQ ID NO: 52 - protein, 242 aa, Trachemys scripta
PDIPEVILKLLQLEPDEPQVKVRILACLQQEQGKGRHEKLSTFGLMCKMADQTLFSIVEWARSCIFFKELEVGDQMKLLQNCWSELLVFDHIYRQVQHGKEHSMLLVTGQEVEMATIAAQAGSNLNNLVLRAQELVLHLHSLQVDRQEFVCLKFLILFSLDVKYLENHSLAKDAQEKANAALLEYTICHYPHAADKFRQLLLRLAEIRSLSMQAEEYLYHKHLSGEVPCNNLLIEMLHAKRT

SEQ ID NO: 53 - protein, 242 aa, Rana rugosa
PDIPEVILKLLQLEPDEPQIKARIISCLQQEQNKSRHEKLSMFGLMCKMADQTLFSIVEWARSCIYFKELEVSDQMILLQNCWSELLVFDHIYRQVQHSKENSILLVTGQEIELSAIAAQAGSTLNNLVLRAQELVILLHSLQVDRQEFVCLKFLILFSLDEKFLENHSLAKSAQEKVDSALMEYTMCHYPHCTDKYRLLLLRLAEIRSISMQAEEYLYHKHLSGEVPCNNLLIEMLHAKRA

SEQ ID NO: 54 - protein, 243 aa, Gallus gallus
ASIPHLILELQKCEPDEPQVQAKIMAYLQQEQANRSKHEKLNTFGLMCKMADQTLFSIVEWARSSIFFRELKVDDQMKLLQNCWSELLILDHIYRQVVHVKEGSILLVTGQQVDYSVIASQAGATLNNLMSHAQELVAKLRSLQFDLREFVCLKFLVLFSLDVKNLENFQLVEGVQEQVNAALLDYTMCNYPQQTDKFGQLLLRLPEIRAISMQAEEYLYCKHLNGDVPCNNLLIEMLHAKRA

SEQ ID NO: 55 - protein, 243 aa, Xenopus laevis
SNIPHLIVELLKCEPDEPQVQGKIMAYLQQEQANRSKHDKLNTFGLMCKMADQTLFSIVEWARSSIFFRDLKVDDQMKLLQNCWSELLILDHIFRQVLHGKEGSILLVTGQQVDYSVIVTQAGATLNNLMSHAQDLVAKLRSLQFDLREFVCLKFLVLFSLDVKTLENYQLVEGVQEQVNAALLDYTMCNYPQQTDKFGQLLLRLPEIRAISLQAEEYLYYKHLNGDVPCNNLLIEMLHAKRA

SEQ ID NO: 56 - protein, 243 aa, Mus musculus
ASIPHLILELLKCEPDEPQVQAKIMAYLQQEQSNRNRQEKLSAFGLLCKMADQTLFSIVEWARSSIFFRELKVDDQMKLLQNCWSELLILDHIYRQVAHGKEGTIFLVTGEHVDYSTIISHTEVAFNNLLSLAQELVVRLRSLQFDQREFVCLKFLVLFSSDVKNLENLQLVEGVQEQVNAALLDYTVCNYPQQTEKFGQLLLRLPEIRAISKQAEDYLYYKHVNGDVPYNNLLIEMLHAKRA

SEQ ID NO: 57 - protein, 243 aa, Rattus norvegicus
ASIPHLILELLKCEPDEPQVQAKIMAYLQQEQNNRNRQEKLSAFGLLCKMADQTLFSIVEWARSSIFFRELKVDDQMKLLQNCWSELLILDHIYRQVAHGKEGTIFLVTGEHVDYSSIISNTEVAFNNLLSLAQELVVRLRSLQFDQREFVCLKFLVLFSSDVKNLENFQLVEGVQEQVNAALLDYTLCNYPQQTEKFGQLLLRLPEIRAISKQAEDYLYYKHVNGDVPYNNLLIEMLHAKRA

SEQ ID NO: 58 - protein, 245 aa, Danio rerio
PALPPLVLELQSCDPDEEQVRGKICAYLHQEQSGRGKLEKSRPSSLLCVMADQTLFSIVEWARSCVFFKELKVGDQMRLLHNCWSELLLLDHICRQVHHGRDGSLLLITGQEVELSAVLDAGPPLSSMVERGQDLSRRLQLLQVDSREMACLKFLILFNPNVKLLENPQFVESVQEQVNGALLEYTLFSYPQCVERFSQLILRLPELRSLSAEAEDFLCYKHLCGEVPCNNLLIEMLHAKGSSAQ

SEQ ID NO: 59 - protein, 237 aa, Drosophila melanogaster
LRVSPMIREFVQSIDDREWQTQLFALLQKQTYNQVEVDLFELMCKVLDQNLFSQVDWARNTVFFKDLKVDDQMKLLQHSWSDMLVLDHLHHRIHNGLPDETQLNNGQVFNLMSLGLLGVPQLGDYFNELQNKLQDLKFDMGDYVCMKFLILLNPSVRGIVNRKTVSEGHDNVQAALLDYTLTCYPSVNDKFRGLVNILPEIHAMAVRGEDHLYTKHCAGSAPTQTLLMEMLHAKRKG

(19) United States
(12) Patent Application Publication
(10) Pub. No.: US 2006/0160135 A1
(43) Pub. Date: Jul. 20, 2006

(54) SF-1 and LRH-1 modulator development

(76) Inventors: Weiru Wang, Lafayette, CA (US); Chao Zhang, Moraga, CA (US); Adhirai Marimuthu, Berkeley, CA (US); Heike I. Krupka, Hayward, CA (US); Maryam Tabrizizad, Milpitas, CA (US); Rafe Shellooe, Concord, CA (US); Upasana Mehra, San Ramon, CA (US); Brian L. West, San Francisco, CA (US)

Correspondence Address: FOLEY & LARDNER LLP, P.O. BOX 80278, SAN DIEGO, CA, 92138-0278, US

(21) Appl. No.: 11/297,793

(22) Filed: Dec. 7, 2005

Related U.S. Application Data
(60) Provisional application No. 60/634,827, filed on Dec. 8, 2004.

(51) Int. Cl. G01N 33/53; G06F 19/00; C07K 14/575

(52) U.S. Cl. 435/7.1; 530/399; 702/19

(57) ABSTRACT

Structures of SF1 and LRH are described, along with methods for identifying or developing modulators of those receptors and uses for such modulators.

### CROSS-REFERENCE TO RELATED PATENT APPLICATION

This application claims the benefit of U.S. Provisional App. No. 60/634,827, filed Dec. 8, 2004, entitled SF-1 and LRH-1 Modulator Development, which is incorporated herein by reference in its entirety and for all purposes.

### FIELD OF THE INVENTION

The present invention relates to the human orphan nuclear receptors steroidogenic factor-1 (SF-1) and liver receptor homolog-1 (LRH-1) and modulation of the activity of those receptors.

### BACKGROUND OF THE INVENTION

The following description is provided solely to assist the understanding of the reader, and does not constitute an admission that any of the information provided or references cited are prior art to the present invention.

Nuclear receptors constitute a protein superfamily whose members specifically bind particular physiologically relevant small molecules, such as hormones or vitamins. As distinguished from integral membrane receptors and membrane-associated receptors, nuclear receptors are located in either the cytoplasm or nucleus of eukaryotic cells.

In many cases of binding of a molecule to a nuclear receptor, the nuclear receptor changes the ability of a cell to transcribe DNA, i.e. nuclear receptors modulate DNA transcription, but can also have transcription independent effects. Thus nuclear receptors comprise a class of intracellular, soluble ligand-regulated transcription factors. Nuclear receptors include but are not limited to receptors for glucocorticoids, androgens, mineralocorticoids, progestins, estrogens, thyroid hormones, vitamin D retinoids, and icosanoids. Many nuclear receptors identified by either sequence homology to known receptors (see, e.g., Drewes et al., Mol. Cell. Biol., 1996, 16:925-31) or based on their affinity for specific DNA binding sites in gene promoters (see, e.g., Sladek et al., Genes & Dev., 1990, 4:2353-65) have unascertained ligands and are therefore termed “orphan receptors.”

In a structural context, nuclear receptors are generally characterized by two distinct structural elements. First, nuclear receptors include a DNA binding domain that targets the receptor to specific DNA sequences, which are known as hormone response elements (HREs). The DNA binding domains of these receptors are related in structure and sequence. Second, the C-terminal region of nuclear receptors encompasses the ligand binding domain (LBD). Upon binding a ligand, the receptor adopts a transcriptionally active state.

Steroidogenic factor-1 (SF-1), also known as adrenal 4-binding protein (Ad4BP) and NR5A1, is an essential factor in adrenal and gonadal development and for the proper functioning of the hypothalamic-pituitary-gonadal axis. SF-1 maps to human gene map locus 9q33. SF-1 is a transcription factor which activates the promoters of various adrenal/gonadal steroid hydroxylase genes, as well as a variety of genes essential for endocrine organogenesis (Ikeda et al., Mol. Endocrinol., 1993, 7:852-860; Morohashi et al., Mol. Endocrinol., 1993, 7:1196-1204; and Parker & Schimmer, Endocr. Rev., 1997, 18:361-377). Mammalian SF-1 exhibits significant similarity to Drosophila fushi tarazu factor 1 (Ftz-F1), a regulator of the developmental homeobox gene fushi tarazu (Lavorgna et al., Science, 1992, 252:848-851; and Ueda et al., Genes & Dev., 1990, 4:624-635). The mouse SF-1 gene therefore has been designated mouse Ftz-F1.

SF-1 is conserved across both vertebrate and invertebrate species, indicating a conserved role for the protein in all metazoans (Honda et al., J. Biol. Chem., 1993, 268:7494-7502; Lala et al., Mol. Endocrinol., 1992, 6:1249-1258; Nomura et al., J. Biol. Chem., 1995, 270:7453-7461; Oba et al., Biochem. Biophys. Res. Comm., 1996, 226:261-267; Sun et al., Dev. Biol., 1994, 162:426-437; and Wong et al., J. Mol. Endocrinol., 1996, 17:139-147). SF-1 homologs have been cloned, for example, from silkworm, chicken and frog as well as a variety of mammalian species.

SF-1 is a member of the steroid receptor superfamily, and all SF-1 homologs have a common structural organization that shares several features with other members of the steroid receptor superfamily. A classic zinc finger DNA-binding domain (DBD) is present in the amino-terminal region; this domain confers high affinity binding to the SF-1 cognate response element and is essential for DNA binding and subsequent transcriptional activation (Wilson et al., Science, 1992, 256:107-110; Wilson et al., Mol. Cell. Biol., 1993, 13:5794-5804). The major nuclear import signal also maps to the tandem zinc finger domain.

In contrast to the majority of steroid receptors, which function as dimers in DNA-binding and transcriptional regulation, SF-1 binds DNA as a monomer at an extended AGGTCA site such as the perfect SF-1 binding site, TCAAGGTCA (Wilson et al., supra, 1993). In SF-1 and other monomeric nuclear receptors, amino acid residues carboxy-terminal to the DNA-binding domain, denoted the “A” box, contribute to binding specificity by recognizing nucleotides 5′ to the AGGTCA response element, resulting in an extended monomer response element with increased binding fidelity (Ueda et al., Mol. Cell. Biol., 1992, 12:5667-5672; Wilson et al., supra, 1992; and Wilson et al., supra, 1993). Such monomeric nuclear receptors include liver related homolog 1/fetoprotein transcription factor (LRH-1/FTF/SF-1.beta.), nerve growth factor-induced gene-B (NGF-IB), estrogen-related receptor 1 (ERR1), estrogen-related receptor 2 (ERR2) and retinoic acid receptor-related orphan nuclear receptor (ROR).

A variety of genes bound and regulated by SF-1 are known in the art. These SF-1 target genes include, for example, steroidogenic enzymes such as cytochrome P450 cholesterol side-chain cleavage enzyme (P450scc) and other steroidogenic targets such as the ACTH receptor; gonadal SF-1 target genes such as the gene for the male-specific Mullerian inhibiting substance (MIS), which is expressed in the Sertoli cells of the testis and responsible for regression of the female specific Mullerian duct; and pituitary and hypothalamic target genes such as αGSU and the luteinizing hormone β subunit (LHβ). A variety of additional SF-1 target genes are known in the art; see, e.g., Hammer & Ingraham, Frontiers in Neurobiology, 1999, 20:199-223.

Like other members of the steroid receptor superfamily, SF-1 contains a conserved ligand-binding domain positioned at the carboxy-terminus of the receptor and a conserved activation function 2 (AF2) sequence in the carboxy-terminal region of the ligand-binding domain. In many nuclear receptors, this domain confers responsiveness to specific ligands that activate or, in some cases, repress receptor transcriptional activity (Evans, Science, 1988, 240:889-895; Forman et al., Nature, 1998, 395:612-615). While SF-1-dependent transcriptional activity has been shown in one instance to exhibit a modest increase in response to 25-, 26-, and 27-hydroxycholesterol in CV-1 cells (Lala et al., Proc. Natl. Acad. Sci. USA, 1997, 94:4895-4900), a ligand for SF-1 has not been definitively identified, and SF-1 consequently is referred to as an “orphan receptor.”

SF-1 has been shown to have transactivating activity in the absence of exogenous ligand. Two regions have been identified as important for SF-1 transactivation. Point mutations within the conserved AF2 hexamer motif, LLIEML, which is critical for transactivation function of many nuclear receptors (Mangelsdorf et al., Cell, 1995, 83:835-839), abrogated SF-1 activity, as did removal of the distal hinge region that follows the DNA-binding domain. In contrast, much of the ligand-binding domain can be truncated without significantly impairing SF-1 transcriptional activity. Furthermore, in cell lines that support SF-1-transcriptional activity, the AF1 domain of SF-1 is constitutively phosphorylated at serine 203. A nonphosphorylatable mutant, SF-1_{S203A}, consistently exhibited a significant 50-80% reduction in transcriptional activity on the MIS promoter and other promoters as compared to wild-type SF-1 activity. Point mutations in the AF2 hexamer motif also resulted in significant reduction in SF-1 transactivation, and a further reduction in activity was observed when the AF2 hexamer mutation was combined with the S203A mutation (Hammer et al., Mol. Cel, 1999, 3:521-526). In sum, maximal SF-1 transcriptional activity requires both the AF1 in the distal hinge domain and AF2 (Crawford et al., Mol. Endocrinol., 1997, 11:1626-1635; Ito et al., Mol. Cell. Biol., 1997, 17:1476-1483). Two motifs in particular, the phosphorylated Ser 203 and LLIEML hexamer of the AF2 domain, are essential for full SF-1 transcriptional activity.

Consistent with a role for SF-1 as a regulator of steroid hydroxylases, SF-1 is expressed in the primary organs that produce steroid hormones, including adrenal cortical cells, testicular Leydig cells, and ovarian theca and granulosa cells (Ikeda et al., Mol. Endocrinol., 1994, 8:654-662; Sasano et al., J. Clin. Endocrinol. Metab., 1995, 80:2378-2380; Takayama et al., J. Clin. Endocrinol. Metab., 1995, 80:2815-2821). SF-1 also is expressed in the testicular Sertoli cell, the pituitary gonadotrope, and the ventral medial nucleus (VMN) of the hypothalamus (Asa et al., J. Clin. Endocrinol. Metab., 1996, 81:2165-2170; Hatano et al., Develop., 1994, 120:2787-2797; Ikeda et al., supra, 1994; Ingraham et al., Genes & Dev., 1994, 8:2302-2312; Morohashi et al., Mol. Endocrinol., 1993, 7:1196-1204; and Roselli et al:, Brain Res. Mol. Brain Res., 1997, 44:66-72). SF-1 transcripts have been detected in spleen and placenta in addition to the gonad, adrenal, pituitary and hypothalamus.

In vivo significance of SF-1 has been demonstrated in SF-1 knockout mice. Homozygous Ftz-F1 −/− mice all died of glucocorticoid and mineralocorticoid insufficiency (Luo et al., Mol. Endocrinol., 1995, 9:1233-1239). The absence of SF-1 resulted in female external genitalia regardless of chromosomal sex, consistent with a role for SF-1 in gonadal formation and synthesis of androgens such as dihydrotestosterone, which is required for development of male external genitalia. Gonads and adrenal glands were completely absent from both sexes. Furthermore, all mice, regardless of chromosomal sex, displayed a female internal reproductive tract (Luo et al., Cell, 1994, 77:481-490; Sadovsky et al., Proc. Natl. Acad. Sci. USA, 1995, 92:10939-10943), consistent with a known role of SF-1 in regulation of Mullerian inhibiting substance (Giuili et al., Development, 1997, 124:1799-1807; Shen et al., Cell, 1994, 77:651-661). In the absence of this inhibitory substance, regression of the Mullerian duct, the precursor of the vagina, uterus and fallopian tube, does not take place. SF-1 null mice also lacked follicle stimulating hormone (FSH) and luteinizing hormone (LH) expression in the anterior pituitary. These results indicate that SF-1 is critical for appropriate development of the adrenals, gonads and pituitary gonadotropes.

The phenotype of the SF-1 null mice parallels the phenotype observed in the human syndrome of X-linked congenital hypoplasia, a disorder which is characterized by hypoplastic adrenal glands often accompanied by profound hypogonadism. The gene responsible for the human syndrome, DAX-1 (dosage-sensitive sex reversal-adrenal hypoplasia congenita critical region on the X chromosome), localizes to Xp21 and, like deletions of SF-1, DAX-1 deletions result in profound adrenal hypoplasia in humans (Muscatelli et al., Nature, 1994, 372:672-676; Zanaria et al., Nature, 1994, 372:635-641). Dax-1 also is an orphan nuclear receptor expressed in multiple endocrine organs; Dax-1 and SF-1 appear to colocalize to cells of the adrenals, gonads, gonadotropes and VMN (Ikeda et al., Mol. Endocrinol., 1995, 9:478-486; Swain et al., Nat. Genetics, 1996, 12:404-409). Together with the similar phenotypes of SF-1 null mice and Dax mutations in humans, these results reinforce the importance of SF-1 and indicate that SF-1 and DAX-1 can work together as essential regulators of the hypothalamic-pituitary-steroidogenesis axis in humans.

Ingraham et al., U.S. Pat. Pub. No. 20040092716, Appl. No. 10/616,897, discusses a properly folded steroidogenic factor-1 (SF-1)-like receptor variant, or active fragment thereof, which has an amino acid sequence that encodes a SF-1 -like receptor variant or active fragment thereof and that lacks at least one naturally occurring cysteine residue within the ligand-binding domain of the receptor. This patent publication also discusses a LRH-1 receptor variant or an active fragment thereof that contains a substitution at particular cysteine residues.

Liver receptor homolog-1 (LRH-1) is a second orphan nuclear receptor that has sequence similarity to SF-1. LRH-1 is expressed in liver, intestine, and pancreas, and acts on genes coordinating bile acid synthesis, enterohepatic circulation, and absorption. Gene knockout and heterozygous loss-of-function studies show that both SF-1 and LRH-1 are essential during embryogenesis for normal development of the organs in which they are expressed, and mammalian cell transfection experiments indicate that SF-1 and LRH-1 function as obligate factors for their target genes, acting apparently constitutively. The mouse LRH-1 structure contains a cavity available for potential ligands, but mutations to fill this cavity did not diminish activity, supporting a model of constitutive, ligand-independent function.

LRH-1 is involved in the regulation of a number of different genes, including, for example, steroidogenic acute regulatory protein (Kim et al., J. Clin Endocrinol Metab., 2004, 89:3042-3047), apolipoprotein Al (Delerive et al., Mol. Endocrinol., 2004, 18:2378-87), cholesterol 7 alpha-hydroxylase (Qin et al., Mol. Endocrinol., 2004, 18:2424-2439), aromatase (Clyne et al., Mol. Cell. Endocrinol., 2004, 215:39-44), carboxyl ester lipase (Fayard et al., J. Biol. Chem., 2003, 278:35725-31), and cytochrome P450 7A.

Zhao et al. U.S. Pat. Pub. No. 20030077664, application Ser. No. 09/922,226 provides methods of screening for compounds that modulate hormone receptor activity in which an isolated receptor-containing complex is assayed for an altered modification state as compared to a control modification state. The presence of an altered modification state serves to identify an effective agent that modulates a biological activity of the nuclear hormone receptor.” Potential receptors mentioned for use in the methods include without limitation RXR, HNF4, TLX, COUP-TF, TR, RAR, PPAR, reverb, ROR, SF-1, LRH-1, EcR, PXR, CAR, NOR1, NURR1, ER, ERR, GR, AR, PR, and MR.

Goodwin et al., U.S. Pat. Pub. No. 2004/0038862, application Ser. No. 10/343,289 concerns a method to identify compounds that modulate bile acid synthesis by assessing the ability of a compound to act as a ligand for short heterodimerizing partner-i or liver receptor homologue-1, preferably a compound that modulates the interaction of short heterodimerizing partner-1 with liver receptor homologue-1.

### SUMMARY OF THE INVENTION

In accordance with the present invention, it has been discovered that “orphan” nuclear receptors human steroidogenic factor-1 (SF-1) and liver receptor homolog-1 (LRH-1) bind phospholipid ligands in a ligand binding domain (LBD) pocket. As a result, the invention provides methods for the identification of modulators that bind in the respective LBD pockets of these receptors.

Thus, in a first aspect, the invention provides a method for identifying compounds that bind to the ligand binding domain of SF-1 or LRH-1 by contacting the ligand binding domain with a test compound and determining whether the compound binds to the domain, thereby identifying compounds that bind to the ligand binding domain of SF-1 or LRH-1. Compounds that bind to the ligand binding domain but do not have detectable modulating activity can be useful for development of derivative compounds that are active modulators, but in preferred embodiments, such binding compounds modulate activity of SF-1 or LRH-1. Thus, such binding compounds can be assayed for modulating activity. The method can be carried out for a plurality of compounds, e.g., a large plurality such as at least 100, 500, 1000, 5000, 10000 compounds. The method additionally contemplates whether the compound binds in a ligand binding pocket. Such a binding determination can be carried out in a variety of ways, e.g., as a direct binding assay or as a competitive assay in which the test compound competes for binding with a known binding compound, e.g., a molecular scaffold as identified herein. The method can also involve determining whether the compound binds at one or both of the co-activator binding surfaces as identified herein. Such a binding determination can be carried out in a variety of ways, e.g., as a direct binding assay or as a competitive assay in which the test compound competes for binding with a known binding compound, e.g., a phospholipid as identified herein.

Identification of such compounds enables a method for identifying or developing additional compounds active on these receptors, e.g., improved modulators. Such identification includes without limitation determining whether any of a plurality of test compounds active on SF-1 or LRH-1 provides an improvement in one or more desired pharmacologic properties relative to an active reference compound. Thereafter, invention methods comprise selecting a compound, if any, that has an improvement in the desired pharmacologic property, thereby providing an improved modulator. In particular embodiments of aspects of modulator development, the desired pharmacologic property is serum half-life longer than 2 hr or longer than 4 hr or longer than 8 hr, aqueous solubility, oral bioavailability more than 10%, or oral bioavailability more than 20%. In certain embodiments, a plurality of derivatives of an active reference compound (e.g., a compound identified in a method described herein) are used.

Also in particular embodiments of aspects of modulator development, the process can be repeated multiple times, i.e., multiple rounds of preparation of derivatives and/or selection of additional related compounds and evaluation of such further derivatives of related compounds, e.g., 1, 2, 3, 4, 5, 6, 7, 8, 9, 10 or more additional rounds.

In another aspect, the invention provides a method of designing a ligand that binds to SF-1 or LRH-1, by identifying one or more molecular scaffolds that bind to a binding site of SF-1 or LRH-1 ligand binding domain polypeptide with low affinity; determining the orientation of the one or more molecular scaffolds at the binding site of the polypeptide by obtaining co-crystal structures of the one or more molecular scaffolds in the binding site; and modifying one or more structures of at least one scaffold molecule so as to provide a ligand having altered binding affinity or binding specificity or both for binding to the polypeptide as compared to the binding of the scaffold molecule. The designed ligand(s) can then be provided, e.g., by synthesizing or otherwise obtaining the ligand(s). In particular embodiments, one or more molecular scaffolds interact with at least 3 conserved amino acid residues in a binding pocket of the ligand binding domain and/or with at least 3 residues with which a phospholipid ligand interacts. In another aspect, the invention provides a method of developing altered modulators for SF-1 or LRH-1 by selecting a molecular scaffold from a set of at least 3 molecular scaffolds that bind to SF-1 or LRH-1, and modifying one or more structures of the scaffold molecule so as to provide a ligand having altered binding affinity or binding specificity or both for binding to SF-1 or LRH-1 as compared to the binding of the scaffold molecule.

In particular embodiments, a plurality of distinct compounds are assayed for binding to the binding site of the SF-1 or LRH-1 ligand binding domain polypeptide; co-crystals of the molecular scaffolds bound to the polypeptide are isolated, and the orientation of the molecular scaffold is determined by performing X-ray crystallography on the co-crystals. In further embodiments, the method involves identifying common chemical structures of the molecular scaffolds, placing the molecular scaffolds into groups based on having at least one common chemical structure, and determining the orientation of the one or more molecular scaffolds at the binding site of the polypeptide for at least one representative compound from a plurality of groups; the ligand binds to the target molecule with greater binding affinity or greater binding specificity or both than the molecular scaffold; the orientation of the molecular scaffold is determined by nuclear magnetic resonance in co-crystal structure determination; the plurality of distinct compounds are each assayed for binding to a plurality of members of the NR5A nuclear receptor family.

Also in particular embodiments, after the identification of common chemical structures of the distinct compounds that bind, the compounds are grouped into classes based on common chemical structures and a representative compound from a plurality of the classes is selected for performing X-ray crystallography on co-crystals of the compound and target molecule; the distinct compounds are selected based on criteria selected from molecular weight, clogP, and the number of hydrogen bond donors and acceptors; the clogP is less than 2, and the number of hydrogen bond donors and acceptors is less than 5.

In certain embodiments, the distinct compounds have a molecular weight of from about 100 to about 350 daltons, or more preferably from about 150 to about 350 daltons or from 150 to 300 daltons, or from 200 to 300 daltons. The distinct compounds can be of a variety of structures. In some embodiments, the distinct compounds can have a ring structure, either a carbocyclic or heterocyclic ring, such as for example, a phenyl ring, a pyrrole, imidazole, pyridine, purine, or any ring structure.

In various embodiments, a compound or compounds binds with extremely low affinity, very low affinity, low affinity, moderate affinity, or high affinity; at least about 5% of the binding compounds bind with low affinity (and/or has low activity), or at least about 10%, 15%, or 20% of the compounds bind with low affinity (or very low or extremely low). After the identification of common chemical structures of the distinct compounds that bind, the compounds can be grouped into classes based on common chemical structures and at least one representative compound from at least one, or preferably a plurality, of the classes selected for performing orientation determination, e.g., by X-ray crystallography and/or NMR analysis.

In selecting the distinct compounds for assay in the present invention, the selection can be based on various criteria appropriate for the particular application, such as molecular weight, clogP (or other method of assessing lipophilicity), Polar Surface Area (PSA) (or other indicator of charge and polarity or related properties), and the number of hydrogen bond donors and acceptors. Compounds can also be selected using the presence of specific chemical moieties which, based on information derived from the molecular family, might be indicated as having some affinity for members of the family. Compounds with highly similar structures and/or properties can be identified and grouped using computational techniques to facilitate the selection of a representative subset of the group. As indicated above, in preferred embodiments, the molecular weight is from about 150 to about 350 daltons, more preferably from 150 to 300 daltons. The clogp is preferably less than 2, the number of hydrogen bond donors and acceptors is preferably less than 5 and the PSA less than 100. Compounds can be selected that include chemical structures of drugs having acceptable pharmacalogical properties and/or lacking chemical structures that are known to result in undesirable pharmacological properties, e.g., excessive toxicity and lack of solubility.

In some embodiments, the assay is an enzymatic assay, and the number of groups of molecular scaffolds formed can conveniently be about 500. In some embodiments, the assay is a competition assay, e.g., a binding competition assay. Cell-based assays can also be used. As indicated above, compounds can be used that have low, very low, or extremely low activity in a biochemical or cell-based assay.

The modification of a molecular scaffold can be the addition, subtraction, or substitution of a chemical group. The modification may desirably cause the scaffold to be actively transported to or into or out of particular cells and/or a particular organ. In various embodiments, the modification of the compound includes the addition or subtraction of a chemical atom, substituent or group, such as, for example, a hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbamylthio, cyclohexyl, pyridyl, piperidinyl, alkylamino, amino, nitro, mercapto, cyano, hydroxyl, a halogen atom, halomethyl, an oxygen atom (e.g., forming a,ketone, ether or N-oxide), and a sulphur atom (e.g., forming a thiol, thione, sulfonamide or di-alkylsulfoxide (sulfone)).

In certain embodiments, the information provided by performing X-ray crystallography on the co-crystals is provided to a computer program, wherein the computer program provides a measure of the interaction between the molecular scaffold and the protein and a prediction of changes in the interaction between the molecular scaffold and the protein that result from specific modifications to the molecular scaffold, and the molecular scaffold is chemically modified based on the prediction of the biochemical result. The computer program can provide the prediction based on a virtual assay such as, for example, virtual docking of the compound to the protein, shape-based matching, molecular dynamics simulations, free energy perturbation studies, and similarity to a three-dimensional pharmacophore. A variety of such programs are well-known in the art.

Chemical modification of a chemically tractable structure can result in, or be selected to provide, one or more physical changes, e.g., to result in a ligand that fills a void volume in the protein-ligand complex, or in an attractive polar interaction being produced in the protein-ligand complex. The modification can also result in a sub-structure of the ligand being present in a binding pocket of the protein binding site when the protein-ligand complex is formed. After common chemical structures of the compounds that bind are identified, the compounds can be grouped based on having a common chemical sub-structure and a representative compound from each group (or a plurality of groups) can be selected for co-crystallization with the protein and performance of the X-ray crystallography. The X-ray crystallography is preferably performed on the co-crystals under distinct environmental conditions, such as at least 20, 30, 40, or 50 distinct environmental conditions, or more preferably under about 96 distinct environmental conditions. The X-ray crystallography and the modification of a chemically tractable structure of the compound can each be performed a plurality of times, e.g., 2, 3, 4, or more rounds of crystallization and modification.

Also in certain embodiments, one or more molecular scaffolds are selected which bind to a plurality of nuclear receptors, such as members of the NR5A group of nuclear receptors.

The method can also include the identification of conserved residues in a binding site(s) of a SF-1 or LRH-1 ligand binding domain polypeptide, that interact with a molecular scaffold, ligand or other binding compound. Conserved residues can, for example, be identified by sequence alignment of different members of the NR5A family and/or homologs of SF-1 or LRH-1, and identifying binding site residues that are the same or at least similar between multiple members of the group. Interacting residues can be characterized as those within a selected distance from the binding compound(s), e.g., 3, 3.5, 4, 4.5, or 5 angstroms.

As used in connection with binding of a compound with a target, the term “interact” indicates that the distance from a bound compound to a particular amino. acid residue will be 5.0 angstroms or less. In particular embodiments, the distance from the compound to the particular amino acid residue is 4.5 angstroms or less, 4.0 angstroms or less, or 3.5 angstroms or less. Such distances can be determined, for example, using co-crystallography, or estimated using computer fitting of a compound in an active site.

In a related aspect, the invention provides a method of designing a ligand that binds to at least one member of the NR5A family, by identifying as molecular scaffolds one or more compounds that bind to binding sites of a plurality of members of the NR5A family, determining the orientation of one or more molecular scaffolds at the binding site of a NR5A receptor(s) to identify chemically tractable structures of the scaffold(s) that, when modified, alter the binding affinity or binding specificity between the scaffold(s) and the receptor(s), and synthesizing a ligand wherein one or more of the chemically tractable structures of the molecular scaffold(s) is modified to provide a ligand that binds to the receptor with altered binding affinity or binding specificity relative to binding of the scaffold.

Particular embodiments include those described for the preceding aspect.

The invention also provides a method to identify interaction properties that a likely SF-1 or LRH-1 binding compound will possess, thereby allowing, for example, more efficient selection of compounds for structure activity relationship determinations and/or for selection for screening. Thus, another aspect concerns a method for identifying binding characteristics of a ligand of a NR5A protein (e.g., SF-1 or LRH-1), by identifying at least one conserved interacting residue in the receptor that interacts with at least two binding compounds; and identifying at least one common interaction property of those binding compounds with the conserved residue(s). The interaction property and location with respect to the structure of the binding compound defines the binding characteristic.

In various embodiments, the identification of conserved interacting residues involves comparing (e.g., by sequence alignment) a plurality of amino acid sequences in the NR5A family and identifying binding site residues conserved in that family; identification of binding site residues by determining co-crystal structure(s); identifying interacting residues (preferably conserved residues) within a selected distance of the binding compounds, e.g., 3, 3.5, 4, 4.5, or 5 angstroms; the interaction property involves hydrophobic interaction, charge-charge interaction, hydrogen bonding, charge-polar interaction, polar-polar interaction, or combinations thereof.

Another related aspect concerns a method for developing ligands for SF-1 or LRH-1 using a set of scaffolds. The method involves selecting one or both of those receptors, selecting a molecular scaffold, or a compound from a scaffold group, from a set of at least 3 scaffolds or scaffold groups where each of the scaffolds or compounds from each scaffold group are known to bind to the target. In particular embodiments, the set of scaffolds or scaffold groups is at least 4, 5, 6, 7, 8, or even more scaffolds or scaffold groups.

In another aspect the invention provides a method of identifying a modulator of a SF-1 or LRH-1 polypeptide by designing or selecting a compound that interacts with amino acid residues in a ligand binding site of the SF-1 or LRH-1 polypeptide, based upon a crystal structure of the respective ligand binding domain polypeptide, e.g., a structure of such a peptide in complex with one or more of a ligand and a coactivator polypeptide. The method can also involve synthesizing the modulator, and/or determining whether the compound modulates the activity of the SF-1 or LRH-1 polypeptide. Compounds that modulate SF-1 or LRH-1 are thus identified as modulators.

In certain embodiments the amino acid residues are conserved residues; are residues that interact with a phospholipid ligand as described herein; include at least 3, 4, 5, 6, or more conserved residues; include at least 3, 4, 5, 6, or more residues that interact with a phospholipid ligand as described herein; or include at least 2, 3, 4, or more residues that, when mutated from wild-type to a non-similar amino acid residue, changes the level of transcription or expression of a gene regulated by SF-1 or LRH-1 by at least 20% in an assay appropriate for determining such transcription or expression level (in particular embodiments, the gene is one identified herein as regulated by SF-1 or LRH-1).

The invention also provides a method of designing a modulator that modulates the activity of a SF-1 or LRH-1 by evaluating the three-dimensional structure of crystallized SF-1 or LRH-1 ligand binding domain polypeptide complexed with one or more of a ligand and a co-activator polypeptide, and synthesizing or selecting a compound based on the three-dimensional structure of the crystal complex that will bind to the polypeptide. Optionally, such a compound binds to the polypeptide as a potential modulator. The method can also involve determining whether the compound modulates the activity of a SF-1 or LRH-1; such determination can include determination of specificity (e.g., specificity between SF-1 and LRH-1, or specificity between SF-1 or LRH-1 and other members of the NR5A nuclear receptor family, or between SF-1 or LRH-1 and other nuclear receptors.

In another aspect, the invention concerns a method of screening for a modulator of SF-1 or LRH-1. The method involves contacting SF-1 or LRH-1 ligand binding domain polypeptide with a plurality of test compounds and determining whether any of the compounds bind with the ligand binding domain polypeptide. The method can also involve determining whether the compound binds in a LBD phospholipid binding pocket or at one or both of the coactivator binding surfaces as identified herein. Such a binding determination can be carried out as a direct binding assay or as a competitive assay in which the test compound competes for binding with a known binding compound, e.g., a phospholipid as identified herein. Test compounds that bind with SF-1 or LRH-1 can also be assayed for ability to modulate SF-1 or LRH-1 activity.

Additional variants of methods for identifying nuclear receptor modulators that can be applied to SF-1 and LRH-1 are described in Bledsoe et al., U.S. Pat. Pub. No. 2004/0018560, application Ser. No. 10/418,007, which is incorporated herein by reference in its entirety.

In another aspect, the invention provides a protein crystal comprising a substantially pure SF1 ligand binding domain polypeptide optionally comprising a ligand, or a LRH-1 ligand binding domain optionally comprising a ligand. In further embodiments of this aspect, the ligand is a phospholipid ligand.

Preferably, the crystalline form has lattice constants as shown in Table 1 and/or has coordinates as specified in Table 2 or Table 3. In certain embodiments, the ligand is a phospholipid.

The invention also provides a method for obtaining a crystal of SF-1 or LRH-1 ligand binding domain by subjecting substantially pure SF-1 or LRH-1 in the presence of a coactivator peptide and/or a ligand (e.g., a phospholipid ligand as described herein) under conditions substantially equivalent to the crystallization conditions described in the Examples herein.

A related aspect concerns a method for determining the three-dimensional structure of a crystallized SF-1 or LRH-1 ligand binding domain polypeptide in complex with one or more of a ligand and a coactivator polypeptide to a resolution of about 2.8 angstroms or better. In certain embodiments, the method includes: (a) crystallizing a SF-1 or LRH-1 ligand binding domain polypeptide in complex with one or more of a ligand and a coactivator polypeptide to form a crystallized complex; and (b) analyzing the crystallized complex to determine the three-dimensional structure of the SF-1 or LRH-1 ligand binding domain polypeptide in complex with one or more of a ligand and a coactivator polypeptide, whereby the three-dimensional structure of a crystallized SF-1 or LRH-1 ligand binding domain polypeptide in complex with one or more of a ligand and a coactivator polypeptide is determined to a resolution of about 2.8 angstroms or better. It is also preferable that the ligand is a phospholipid, e.g., as described herein.

The invention also provides a modified SF-1 or LRH-1 ligand binding domain, e.g., a domain which is modified as described in the Examples herein. In particular embodiments, the domain is SF-1 ligand binding domain which is modified by substitution or deletion of surface cysteines, C247 and/or C412. The modification can be substitution by serine residues.

As is conventional, the terms “a” and “an” mean “one or more” when used herein, including in the claims.

As used herein, the term “expression” generally refers to the cellular processes by which a polypeptide is produced from RNA.

As used herein, the term “transcription factor” means a cytoplasmic or nuclear protein which binds to a gene, or binds to an RNA transcript of a gene, or binds to another protein which binds to a gene or an RNA transcript or another protein which in turn binds to a gene or an RNA transcript, so as to thereby modulate expression of the gene. Such modulation can additionally be achieved by other mechanisms; the essence of a “transcription factor for a gene” pertains to a factor that alters the level of transcription of the gene in some way.

As used herein in connection with polynucleotides and polypeptides, the term “isolated” means that the molecule is separated from a substantial amount of other nucleic acids, proteins, lipids, carbohydrates or other materials with which they associate, such association being either in cellular material or in a synthesis medium. For example, the polynucleotide or polypeptide can be separated from 50, 60, 70, 80, 90, 95, 97, 98, 99% or more of such other materials.

As used herein, the term “substantially pure” means that the polynucleotide or polypeptide is substantially free of other polynucleotides and/or polypeptides, and thus constitutes at least 50, 60, 70, 80, 90, 95, 97, 98, 99% or more of a sample or preparation as the substantially pure polynucleotide or polypeptide.

As used herein, the term “modified” means an alteration from an entity's normally occurring state. An entity can be modified by removing discrete chemical units or by adding discrete chemical units. The term “modified” encompasses detectable labels as well as those entities added as aids in purification and entities added or removed as aids in crystallization.

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

As used herein, the term “space group” means the arrangement of symmetry elements of a crystal.

As used herein, the term “molecular replacement” means a method that involves generating a preliminary model of, for example, the wild-type SF-1 ligand binding domain, or a SF-1 mutant crystal whose structure coordinates are unknown, by orienting and positioning a molecule whose structure coordinates are known within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See, e.g., Lattman, 1985, Method Enzymol., 115: 55-77; Rossmann (ed.), 1972, The Molecular Replacement Method, Gordon & Breach, New York. Using the structure coordinates of a SF-1 or LRH-1 ligand binding domain provided by the present invention, molecular replacement can be used to determine the structure coordinates of a crystalline mutant or homologue of a SF-1 or LRH-1 ligand binding domain, or of a different crystal form of the SF-1 or LRH-1 ligand binding domain.

As used herein, the term “isomorphous replacement” means a method of using heavy atom derivative crystals to obtain the phase information necessary to elucidate the three-dimensional structure of a native crystal (Blundell et al., Protein Crystallography, 1976, Academic Press; Otwinowski, in Isomorphous Replacement and Anomalous Scattering, (Evans & Leslie, eds.), 1991, 80-86, Daresbury Laboratory, Daresbury, United Kingdom). The phrase “heavy-atom derivatization” is synonymous with the term “isomorphous replacement.”

As used herein, the term “polypeptide” means a polymer of amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless clearly indicated to the contrary. As used herein, the terms “protein”, “polypeptide” and “peptide” are used interchangeably herein when referring to a gene product.

As used herein, the term “modulate” means an increase, decrease, or other alteration of any, or all, chemical and biological activities or properties of a wild-type or mutant SF-1 or LRH-1 polypeptide. The term “modulation” as used herein refers to both upregulation (i.e., activation or stimulation) and downregulation (i.e. inhibition or suppression) of a response. Thus a modulator may be either an agonist or an antagonist.

As used herein, the term “gene” is used for simplicity to refer to a functional protein, polypeptide or peptide encoding unit. As will be understood by those in the art, this functional term includes both genomic sequences and cDNA sequences.

As used herein, the term “intron” means a DNA sequence present in a given gene that is not translated into protein.

As used herein, the term “agonist” means an agent that increases, supplements, or potentiates the bioactivity of a functional gene or protein, e.g., SF-1 or LRH-1.

As used herein, the term “antagonist” means an agent that decreases or inhibits the bioactivity of a functional gene or protein, e.g., SF-1 or LRH-1.

As used herein in connection with SF-1 and LRH-1 modulating compounds, binding compounds or ligands, the term “specific for SF-1”, “specific for LRH-1” and terms of like import mean that a particular compound binds to the specified receptor to a statistically greater extent than to other biomolecules that may be present in a particular organism, e.g., at least 2, 3, 4, 5, 10, 20, 50, 100, or 1000-fold. Also, where biological activity other than binding is indicated, the term “specific for SF-1” or “specific for LRH-1” indicates that a particular compound has greater biological activity associated with binding to the specified receptor than to other biomolecules (e.g., at a level as indicated for binding specificity). Similarly, the specificity can be for the specific receptor with respect to other nuclear receptors that may be present from an organism. In particular embodiments, the specificity is between SF-1 and LRH-1.

As used herein, the terms “ligand” and “modulator” are used equivalently to refer to a compound that alters the activity of a target biomolecule, e.g., SF-1 or LRH-1. Generally a ligand or modulator will be a small molecule, where “small molecule refers to a compound with a molecular weight of 1500 daltons or less, or preferably 1000 daltons or less, 800 daltons or less, or 600 daltons or less. Thus, an “improved ligand” is one that possesses better pharmacological and/or pharmacokinetic properties than a reference compound, where “better” can be defined by a person for a particular biological system or therapeutic use. In terms of the development of ligands from scaffolds, a ligand is a derivative of a molecular scaffold that has been chemically modified at one or more chemically tractable structures to bind to the target molecule with altered or changed binding affinity or binding specificity relative to the molecular scaffold. The ligand can bind with a greater specificity and/or affinity for a member of the molecular family relative to the molecular scaffold. A ligand binds non-covalently to a target molecule, which can preferably be a protein or enzyme.

In the context of binding compounds, molecular scaffolds, and ligands, the term “derivative” or “derivative compound” refers to a compound having a common core chemical structure relative to a parent or reference compound, but differs by having at least one structural difference, e.g., by having one or more substituents added and/or removed and/or substituted, and/or by having one or more atoms substituted with different atoms. Unless clearly indicated to the contrary, the term “derivative” does not mean that the derivative is synthesized using the parent compound as a starting material or as an intermediate, although in some cases, the derivative may be synthesized from the parent.

Thus, the term “parent compound” refers to a reference compound for another compound, having structural features also present in the derivative compound. Often but not always, a parent compound has a simpler chemical structure than the derivative.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. In some cases, the specificity is with reference to a limited set of other biomolecules, e.g., in the case of SF-1 and LRH-1, in some cases the reference may be other nuclear receptors, or for SF-1 it may be LRH-1 and for LRH-1 it may be SF-1. In particular embodiments, the greater specificity is at least 2, 3, 4, 5, 8, 10, 50, 100, 200, 400, 500, or 1000-fold greater specificity.

Another aspect of the invention concerns novel compounds that bind to a ligand binding domain of SF-1 or LRH-1 and make interactions with amino acids in the ligand binding domain pocket that interact with the phospholipids identified herein.

A related aspect of this invention concerns pharmaceutical compositions that include such a binding compound and at least one pharmaceutically acceptable carrier, excipient, or diluent. The composition can include a plurality of different pharmacologically active compounds.

As used herein, the term “pharmaceutical composition” refers to a preparation that includes a therapeutically significant quantity of an active agent, that is prepared in a form adapted for administration to a subject. Thus, the preparation does not include any component or components in such quantity that a reasonably prudent medical practitioner would find the preparation unsuitable for administration to a normal subject. In many cases, such a pharmaceutical composition is a sterile preparation.

In a related aspect, the invention provides kits that include a pharmaceutical composition as described herein. In particular embodiments, the pharmaceutical composition is packaged, e.g., in a vial, bottle, flask, which may be further packaged, e.g., within a box, envelope, or bag; the pharmaceutical composition is approved by the U.S. Food and Drug Administration or similar regulatory agency for administration to a mammal, e.g., a human; the pharmaceutical composition is approved for administration to a mammal, e.g., a human for a SF-1- or LRH-1-mediated disease or condition; the kit includes written instructions or other indication that the composition is suitable or approved for administration to a mammal, e.g., a human, for a SF-1- or LRH-1-mediated disease or condition; the pharmaceutical composition is packaged in unit dose or single dose form, e.g., single dose pills, capsules, or the like.

In another related aspect, such binding compounds can be used in the preparation of a medicament for the treatment of a SF-1- or LRH-1-mediated disease or condition or a disease or condition in which modulation of one of those nuclear receptors provides a therapeutic benefit.

In another aspect, the invention concerns a method of treating or prophylaxis of a disease or condition in a mammal, e.g., a SF-1- or LRH-1-mediated disease or condition or a disease or condition in which modulation of one of those receptors provides a therapeutic benefit, by administering to the mammal a therapeutically effective amount of a compound that binds in the ligand binding domain pocket, a prodrug of such compound, or a pharmaceutically acceptable salt of such compound or prodrug. The compound can be alone or can be part of a pharmaceutical composition. In a further embodiment, the invention provides a method of treating or prophylaxis of a disease or condition in a mammal, e.g., a SF-1- or LRH-1-mediated disease or condition or a disease or condition in which modulation of one of those receptors provides a therapeutic benefit, by administering to the mammal a therapeutically effective amount of a compound that modulates the activity of SF-1 or LRH-1, a prodrug of such compound, or a pharmaceutically acceptable salt of such compound or prodrug. In a preferred embodiment, the SF-1 or LRH-1 modulator is designed according to a method for designing a ligand that binds to SF-1 or LRH-1 as described herein.

In aspects and embodiments involving treatment or prophylaxis of a disease or conditions, the disease or condition includes without limitation elevated cholesterol level, cancer, hepatitis virus infection, improper or risk of improper development.

As used herein, the terms “SF-1-mediated” and “LRH-1-mediated” disease or condition and like terms refer to a disease or condition in which the biological function of the specified receptor affects the development and/or course of the disease or condition, and/or in which modulation of the receptor alters the development, course, and/or symptoms of the disease or condition. Similarly, the phrases “SF-1 modulation provides a therapeutic benefit” and “LRH-1 modulation provides a therapeutic benefit” and the like indicate that modulation of the level of activity of the specified receptor in a subject indicates that such modulation reduces the severity and/or duration of the disease, reduces the likelihood or delays the onset of the disease or condition, and/or causes an improvement in one or more symptoms of the disease or condition.

In the present context, the term “therapeutically effective” indicates that the materials or amount of material are effective to prevent, alleviate, or ameliorate one or more symptoms of a disease or medical condition, and/or to prolong the survival of the subject being treated.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectibles.

“A pharmaceutically acceptable salt” is intended to mean a salt that retains the biological effectiveness of the free acids and bases of the specified compound and that is not biologically or otherwise unacceptable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sodium, chloride, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, .gamma.-hydroxybutyrates, glycollates, tartrates, methane-sulfonates, propanesulfonates, naphthalene-1 -sulfonates, naphthalene-2-sulfonates, and mandelates.

The term “pharmaceutically acceptable metabolite” refers to a pharmacologically acceptable product, which may be an active product, produced through metabolism of a specified compound (or salt thereof) in the body of a subject or patient. Metabolites of a compound may be identified using routine techniques known in the art, and their activities determined using tests such as those described herein. For example, in some compounds, one or more alkoxy groups can be metabolized to hydroxyl groups while retaining pharmacologic activity and/or carboxyl groups can be esterified, e.g., glucuronidation. In some cases, there can be more than one metabolite, where an intermediate metabolite(s) is further metabolized to provide an active metabolite. For example, in some cases a derivative compound resulting from metabolic glucuronidation may be inactive or of low activity, and can be further metabolized to provide an active metabolite.

In another aspect, the invention provides a method for identifying structurally and energetically allowed sites on a binding compound for attachment of an additional component(s) by analyzing the orientation of the binding compound(s) in a SF-1 or LRH-1 binding site (e.g., by analyzing co-crystal structures), thereby identifying accessible sites on the compound for attachment of the additional component. In particular embodiments, the binding compound is a phospholipid, e.g., as described herein.

In various embodiments, the method involves calculating the change in binding energy on attachment of the additional component at one or more of the accessible sites; the orientation is determined by co-crystallography; the additional component includes a linker, a label such as a fluorophore, a solid phase material such as a gel, bead, plate, chip, or well.

In a related aspect, the invention provides a method for attaching a SF-1 or LRH-1 binding compound to an attachment component(s) without substantially altering the ability of the SF-1 or LRH-1 binding compound to bind SF-1 or LRH-1, by identifying energetically allowed sites for attachment of such an attachment component on a binding compound (e.g., as described for the preceding aspect), and attaching the binding compound or derivative thereof to the attachment component(s) at the energetically allowed site(s). In particular embodiments, the binding compound is a phospholipid as identified herein.

In various embodiments, the attachment component is a linker (which can be a traceless linker) for attachment to a solid phase medium, and the method also involves attaching the binding compound or derivative to a solid phase medium through the linker attached at the energetically allowed site; the binding compound or derivative thereof is synthesized on a linker attached to the solid phase medium; a plurality of compounds or derivatives are synthesized in combinatorial synthesis; the attachment of the compound(s) to the solid phase medium provides an affinity medium

In a related aspect, the invention provides a method for making an affinity matrix for SF-1 or LRH-1, where the method involves identifying energetically allowed sites on a SF-1 or LRH-1 binding compound for attachment to a solid phase matrix without substantially altering the ability of the SF-1 or LRH-1 binding compound to bind SF-1 or LRH-1; and attaching the binding compound to the solid phase matrix through the energetically allowed site. In particular embodiments, the binding compound is a phospholipid, e.g., as described herein.

Various embodiments are as described for attachment of an additional component above; identifying energetically allowed sites for attachment to a solid phase matrix is performed for at least 5, 10, 20, 30, 50, 80, or 100 different compounds; identifying energetically allowed sites is performed for molecular scaffolds or other SF-1 or LRH-1 binding compounds.

SF-1 homologs can be identified by their sequences, where exemplary reference sequence accession numbers are NM_{—}004959 (cDNA sequence for hSF-1) (SEQ ID NO:______) and NP_{—}004950 (protein sequence for hSF-1) (SEQ ID NO:______). One of ordinary skill in the art will recognize that sequence differences will exist due to allelic variation, and will also recognize that other animals, particularly other mammals, have corresponding receptors, which have been identified or can be readily identified using sequence alignment and confirmation of activity, which can also be used. A number of such sequences are readily available from GenBank. One of ordinary skill in the art will also recognize that modifications can be introduced in a SF-1 sequence without destroying receptor activity. Such modified receptors can also be used in the present invention, e.g., if the modifications do not alter the binding site conformation to the extent that the modified receptor lacks substantially normal ligand binding.

As used herein, the terms “steroidogenic factor 1 ligand binding domain polypeptide”, “SF-1 ligand binding domain polypeptide”, and “SF-1 LBD polypeptide” (and like terms) refer to a polypeptide that contains the site where phospholipid binding as identified herein occurs. For human SF-1, such domain generally includes residues P221 through T461 of NP_{—}004950. An exemplary such domain polypeptide is the polypeptide used for crystallization herein consisting of residues G219 to T461 of NP_{—}004950; additional examples include homologs and variants thereof.

LRH-1 homologs can be identified by their sequences, where exemplary reference sequence accession numbers are NM_{—}003822 (cDNA sequence for hLRH-1 isoform 2) (SEQ ID NO:______), NP_{—}003813 (protein sequence for HLRH-1 isoform 2) (SEQ ID NO:______), NM_{—}205860 (cDNA sequence for hLRH-1 isoform 1) (SEQ ID NO:______), and NP_{—}995582 (protein sequence for hLRH-1 isoform 1) (SEQ ID NO:______). One of ordinary skill in the art will recognize that sequence differences will exist due to allelic variation, and will also recognize that other animals, particularly other mammals, have corresponding receptors, which have been identified or can be readily identified using sequence alignment and confirmation of activity, which can also be used. A number of such sequences are readily available from GenBank. One of ordinary skill in the art will also recognize that modifications can be introduced in a LRH-1 sequence without destroying receptor activity. Such modified receptors can also be used in the present invention, e.g., if the modifications do not alter the binding site conformation to the extent that the modified receptor lacks substantially normal ligand binding.

As used herein, the terms “liver receptor homolog 1 ligand binding domain polypeptide”, “LRH-1 ligand binding domain polypeptide”, and “LRH-1 LBD polypeptide” (and like terms) refer to a polypeptide that contains the site where phospholipid binding as identified herein occurs. For human LRH-1, such domain generally includes residues A253 through A495 of NP_{—}003813 encoded by NM_{—}003822 (supra). For mouse LRH-1, such sequence generally extends from A318 through A560 of the protein encoded by NM_{—}030676 (SEQ ID NO:______). An exemplary such human domain polypeptide is the polypeptide used for crystallization herein consisting of residues S251-A495 of NP_{—}003822 (supra); additional examples include homologs and variants thereof.

As used herein in connection with the design or development of ligands, the term “bind” and “binding” and like terms refer to a non-covalent energetically favorable association between the specified molecules (i.e., the bound state has a lower free energy than the separated state, which can be measured calorimetrically). For binding to a target, the binding is at least selective, that is, the compound binds preferentially to a particular target or to members of a target family at a binding site, as compared to non-specific binding to unrelated proteins not having a similar binding site. For example, BSA is often used for evaluating or controlling non-specific binding. In addition, for an association to be regarded as binding, the decrease in free energy going from a separated state to the bound state must be sufficient so that the association is detectable in a biochemical assay suitable for the molecules involved.

By “assaying” is meant the creation of experimental conditions and the gathering of data regarding a particular result of the experimental conditions. For example, enzymes can be assayed based on their ability to act upon a detectable substrate. Likewise, for example, a compound or ligand can be assayed based on its ability to bind to a particular target molecule or molecules and/or to modulate an activity of a target molecule.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

When a decision is described as “based on” particular criteria, it is meant that the criteria selected are parameters of the decision and guide its outcome. A substantial change in the parameters is likely to result in a change in the decision.

By “binding site” is meant an area of a target molecule to which a ligand can bind non-covalently. Binding sites embody particular shapes and often contain multiple binding pockets present within the binding site. The particular shapes are often conserved within a class of molecules, such as a molecular family. Binding sites within a class also can contain conserved structures such as, for example, chemical moieties, the presence of a binding pocket, and/or an electrostatic charge at the binding site or some portion of the binding site, all of which can influence the shape of the binding site.

By “binding pocket” is meant a specific region of space within a binding site. A binding pocket is a particular space within a binding site at least partially bounded by target molecule atoms. Thus a binding pocket is a particular shape, indentation, or cavity in the binding site. Binding pockets can contain particular chemical groups or structures that are important in the non-covalent binding of another molecule such as, for example, groups that contribute to ionic, hydrogen bonding, van der Waals, or hydrophobic interactions between the molecules.

By “chemical structure” or “chemical substructure” is meant any definable atom or group of atoms that constitute a part of a molecule. Normally, chemical substructures of a scaffold or ligand can have a role in binding of the scaffold or ligand to a target molecule, or can influence the three-dimensional shape, electrostatic charge, and/or conformational properties of the scaffold or ligand.

By “orientation” in reference to a binding compound bound to a target molecule is meant the spatial relationship of the binding compound and at least some of its constituent atoms to the binding pocket and/or atoms of the target molecule at least partially defining the binding pocket.

In the context of target molecules in the present invention, the term “crystal” refers to an ordered complex of target molecule, such that the complex produces an X-ray diffraction pattern when placed in an X-ray beam. Thus, a “crystal” is distinguished from a disordered or partially ordered complex or aggregate of molecules that do not produce such a diffraction pattern. Preferably a crystal is of sufficient order and size to be useful for X-ray crystallography. A crystal may be formed only of target molecule (with solvent and ions) or may be a co-crystal of more than one molecule, for example, as a co-crystal of target molecule and binding compound, and/or of a complex of proteins (such as a holoenzyme).

In the context of this invention, unless otherwise specified, by “co-crystals” is meant an ordered complex of the compound, molecular scaffold, or ligand bound non-covalently to the target molecule that produces a diffraction pattern when placed in an X-ray beam. Preferably the co-crystal is in a form appropriate for analysis by X-ray or protein crystallography. In preferred embodiments the target molecule-ligand complex can be a protein-ligand complex.

By “clogP” is meant the calculated log P of a compound, “P” referring to the partition coefficient of the compound between a lipophilic and an aqueous phase, usually between octanol and water.

By “chemically tractable structures” is meant chemical structures, sub-structures, or sites on a molecule that can be covalently modified to produce a ligand with a more desirable property. The desirable property will depend on the needs of the particular situation. The property can be, for example, that the ligand binds with greater affinity to a target molecule, binds with more specificity, or binds to a larger or smaller number of target molecules in a molecular family, or other desirable properties as needs require.

In the context of compounds binding to a target, the term “greater affinity” indicates that the compound binds more tightly than a reference compound, or than the same compound in a reference condition, i.e., with a lower dissociation constant. In particular embodiments, the greater affinity is at least 2, 3, 4, 5, 8, 10, 50, 100, 200, 400, 500, 1000, or 10,000-fold greater affinity.

By “designing a ligand,” “preparing a ligand,” “discovering a ligand,” and like phrases is meant the process of considering relevant data (especially, but not limited to, any individual or combination of binding data, X-ray co-crystallography data, molecular weight, clogP, and the number of hydrogen bond donors and acceptors) and making decisions about advantages that can be achieved as a result of specific structural modifications to a molecule, and implementing those decisions. This process of gathering data and making decisions about structural modifications that can be advantageous, implementing those decisions, and determining the result can be repeated as many times as necessary to obtain a ligand with desired properties.

By “docking” is meant the process of attempting to fit a three-dimensional configuration of a binding pair member into a three-dimensional configuration of the binding site or binding pocket of the partner binding pair member, which can be a protein, and determining the extent to which a fit is obtained. The extent to which a fit is obtained can depend on the amount of void volume in the resulting binding pair complex (or target molecule-ligand complex). The configuration can be physical or a representative configuration of the binding pair member, e.g., an in silico representation or other model.

By binding with “low affinity” is meant binding to the target molecule with a dissociation constant (K_{D}) of greater than 1 μM under standard conditions. In particular cases, low affinity binding is in a range of 1 μM-10 mM, 1 μM-1 mM, 1 μM-500 μM, 1 μM-200 μM, 1 μM-100 μM. By binding with “very low affinity” is meant binding with a K_{D} of above about 100 μM under standard conditions, e.g., in a range of 100 μM-1 mM, 100 μM-500 μM, 100 μM-200 μM. By binding with “extremely low affinity” is meant binding at a K_{D} of above about 1 mM under standard conditions. By “moderate affinity” is meant binding with a K_{D} of from about 200 nM to about 1 μM under standard conditions. By “moderately high affinity” is meant binding at a K_{D} of from about 1 nM to about 200 nM. By binding at “high affinity” is meant binding at a K_{D} of below about 1 nM under standard conditions. For example, low affinity binding can occur because of a poorer fit into the binding site of the target molecule or because of a smaller number of non-covalent bonds, or weaker covalent bonds present to cause binding of the scaffold or ligand to the binding site of the target molecule relative to instances where higher affinity binding occurs. The standard conditions for binding are at pH 7.2 at 37° C. for one hour. For example, 100 μl/well can be used in HEPES 50 mM buffer at pH 7.2, NaCl 15 mM, ATP 2 μM, and bovine serum albumin 1 ug/well, 37° C. for one hour.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured. For SF-1 and LRH-1 agonists and antagonists, activities can be determined as described in the Examples, or using other such assay methods as described herein or known in the art.

By “molecular scaffold” or “scaffold” is meant a small target binding molecule to which one or more additional chemical moieties can be covalently attached, modified, or eliminated to form a plurality of molecules with common structural elements. The moieties can include, but are not limited to, a halogen atom, a hydroxyl group, a methyl group, a nitro group, a carboxyl group, or any other type of molecular group including, but not limited to, those recited in this application. Molecular scaffolds bind to at least one target molecule with low or very low affinity and/or bind to a plurality of molecules in a target family (e.g., protein family), and the target molecule is preferably an enzyme, receptor, or other protein. Preferred characteristics of a scaffold include molecular weight of less than about 350 daltons; binding at a target molecule binding site such that one or more substituents on the scaffold are situated in binding pockets in the target molecule binding site; having chemically tractable structures that can be chemically modified, particularly by synthetic reactions, so that a combinatorial library can be easily constructed; having chemical positions where moieties can be attached that do not interfere with binding of the scaffold to a protein binding site, such that the scaffold or library members can be modified to form ligands, to achieve additional desirable characteristics, e.g., enabling the ligand to be actively transported into cells and/or to specific organs, or enabling the ligand to be attached to a chromatography column for additional analysis. Thus, a molecular scaffold is a small, identified target binding molecule prior to modification to improve binding affinity and/or specificity, or other pharmacalogic properties.

The term “scaffold core” refers to the core structure of a molecular scaffold onto which various substituents can be attached. Thus, for a number of scaffold molecules of a particular chemical class, the scaffold core is common to all the scaffold molecules. In many cases, the scaffold core will consist of or include one or more ring structures.

The term “scaffold group” refers to a set of compounds that share a scaffold core and thus can all be regarded as derivatives of one scaffold molecule.

By “molecular family” is meant groups of molecules classed together based on structural and/or functional similarities. Examples of molecular families include proteins, enzymes, polypeptides, receptor molecules, oligosaccharides, nucleic acids, DNA, RNA, etc. Thus, for example, a protein family is a molecular family. Molecules can also be classed together into a family based on, for example, homology. The person of ordinary skill in the art will realize many other molecules that can be classified as members of a molecular family based on similarities in chemical structure or biological function.

By “protein-ligand complex” or “co-complex” is meant a protein and ligand bound non-covalently together.

By “protein” is meant a polymer of amino acids. The amino acids can be naturally or non-naturally occurring. Proteins can also contain adaptations, such as being glycosylated, phosphorylated, or other common modifications.

By “protein family” is meant a classification of proteins based on structural and/or functional similarities. For example, kinases, phosphatases, proteases, and similar groupings of proteins are protein families. Proteins can be grouped into a protein family based on having one or more protein folds in common, a substantial similarity in shape among folds of the proteins, homology, or based on having a common function. In many cases, smaller families will be specified, e.g., the nuclear receptor family or the NR5A nuclear receptor family.

“Protein folds” are 3-dimensional shapes exhibited by the protein and defined by the existence, number, and location in the protein of alpha helices, beta-sheets, and loops, i.e., the basic secondary structures of protein molecules. Folds can be, for example, domains or partial domains of a particular protein.

By “ring structure” is meant a molecule having a chemical ring or sub-structure that is a chemical ring. In most cases, ring structures will be carbocyclic or heterocyclic rings. The chemical ring may be, but is not limited to, a phenyl ring, aryl ring, pyrrole ring, imidazole, pyridine, purine, or any ring structure.

By “specific biochemical effect” is meant a therapeutically significant biochemical change in a biological system causing a detectable result. This specific biochemical effect can be, for example, the inhibition or activation of an enzyme, the inhibition or activation of a protein that binds to a desired target, or similar types of changes in the body's biochemistry. The specific biochemical effect can cause alleviation of symptoms of a disease or condition or another desirable effect. The detectable result can also be detected through an intermediate step.

By “standard conditions” is meant conditions under which an assay is performed to obtain scientifically meaningful data. Standard conditions are dependent on the particular assay, and can be generally subjective. Normally the standard conditions of an assay will be those conditions that are optimal for obtaining useful data from the particular assay. The standard conditions will generally minimize background signal and maximize the signal sought to be detected.

By “standard deviation” is meant the square root of the variance. The variance is a measure of how spread out a distribution is. It is computed as the average squared deviation of each number from its mean. For example, for the numbers 1, 2, and 3, the mean is 2 and the variance is 0.667; viz,

$${σ}^{2} = \frac{{( 1 - 2 )}^{2} + {( 2 - 2 )}^{2} + {( 3 - 2 )}^{2}}{3} = 0.667 .$$

By a “set” of compounds is meant a collection of compounds. The compounds may or may not be structurally related.

In the context of this invention, by “target molecule” is meant a molecule that a compound, molecular scaffold, or ligand is being assayed for binding to. The target molecule has an activity that binding of the molecular scaffold or ligand to the target molecule will alter or change. The binding of the compound, scaffold, or ligand to the target molecule can preferably cause a specific biochemical effect when it occurs in a biological system. A “biological system” includes, but is not limited to, a living system such as a human, animal, plant, or insect. In most but not all cases, the target molecule will be a protein or nucleic acid molecule.

By “pharmacophore” is meant a representation of molecular features that are considered to be responsible for a desired activity, such as interacting or binding with a receptor. A pharmacophore can include 3-dimensional (hydrophobic groups, charged/ionizable groups, hydrogen bond donors/acceptors), 2D (substructures), and ID (physical or biological) properties.

As used herein in connection with numerical values, the terms “approximately” and “about” mean ±10% of the indicated value.

Additional aspects and embodiments will be apparent from the following Detailed Description and from the claims.

### BRIEF DESCRIPTION OF THE DRAWINGS

FIGS. 1A-1D schematically shows the human SF-1 and LRH-1 LBD structures complexed with phospholipid and coactivator peptide. A) The human SF-1 LBD (ribbon model), with phospholipid ligand (spherical model), and NCoA-2 coactivator peptide (ribbon model, dark, surrounded by H3, H4 and H12). B) The human LRH-1 LBD, with phospholipid ligand and NCoA-2 coactivator peptides (coded as in (A)). Note that two NCoA-2 peptides bind to each human LRH-1 molecule, one at the canonical activation function surface (H3, H4 and H12), and the other at the site formed by H2, H3 and the β-sheet (lower right corner of figure). C) Residues of the human SF-1 ligand binding pocket (stick models), showing salt-bridge and hydrogen-bonds (dotted lines) to the PE (stick models). The mesh indicates an unbiased 2Fo-Fc map covering the ligand. H2 and H3 are truncated to show the pocket features. D) Residues of the human LRH-1 ligand binding pocket, depicted as in (C), showing interactions with the PG.

FIGS. 2A-2B schematically shows LBD binding pocket residues that interact with ligand for human SF-1 and LRH-1. Residues making hydrophobic contacts are selected generally using a 4.1 A distance cutoff between carbon atoms. A) human SF-1 contacting PE. B) human LRH-1 contacting PG.

FIGS. 3A-3B shows that the human SF-1 and LRH-1 LBD pocket contours filled with ligand, except for a conserved polar pocket. A) The human SF-1 LBD pocket surface contour (represented by the mesh), calculated using a 1.4A radius ball (Kleywegt, 1994, Acta Crystallogr D Biol Crystallogr 50, 178-85), with a volume of ˜550 Å³. Shown are the SF-1 LBD and coactivator peptide mainchains (ribbon), and the PE molecule (molecular surface). The amine of the PE extends toward the exterior of the pocket, and thus extends outside the mesh. Water molecules (dark spheres) are present in a polar pocket. B) The human LRH-1 ligand pocket surface contour, with a volume of 510 Å³, with PG molecule, depicted as in (A).

FIGS. 4A-4C compares the human SF-1 and LRH-1 structures with the mouse LRH-1 structure. A) The phosphate group of PE interacts with K440, Y436, and G341 of the KYG triad in human SF-1. B) The phosphate group of PG interacts with K474, Y470, and G375 of the KYG triad in human LHR-1. C) E440 in the apo mouse LRH-1 mimics the phosphate group interactions. Only the residues of the phosphate-binding triad and the polar portions of the phospholipids are shown (sticks).

FIG. 5 shows an alignment of various NR5A subfamily LBD sequences. The human SF-1 sequence extends from P221 through T461 [NP_{—}004950 (SEQ ID NO:______) encoded by NM_{—}004959 (SEQ ID NO:______)]; the human LRH-1 sequence extends from A253 through A495 [NP_{—}003813 (SEQ ID NO:______) encoded by NM_{—}003822 (SEQ ID NO:______)]; and the mouse LRH-1 sequence extends from A318 through A560 [encoded by NM_{—}030676 (SEQ ID NO:______)]. The secondary structure features are indicated above the sequences. Shading indicates residues identical in at least 11 of 12 aligned sequences. The pocket residues contacting the ligands are indicated by asterisk. The surface residues constituting the canonical AF-2 surface are indicated by the number 1, and the novel second coactivator-binding site by the number 2. The four phosphate-nucleating residues are indicated by rectangles.

FIGS. 6A-6D shows mass spectral analysis of lipids bound to human SF-1 and LRH-1 LBD proteins purified from E. coli: A) wild-type SF-1, B) SF-1/Y436F-K440A, C) wild-type LRH-1, and D) LRH-1/Y470F-K474A. The analyses were performed in negative mode. PE-12:0 (50 pmol) was mixed with 50 pmol of each LBD protein before extraction, giving the m/z=578 standard peak.

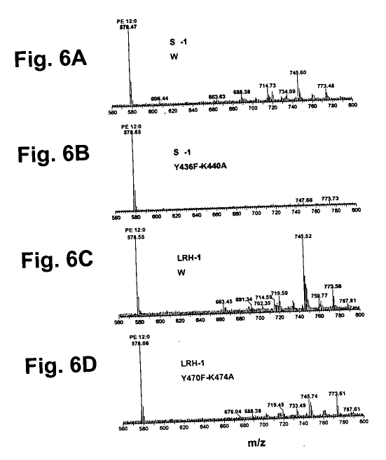

FIGS. 7A-7B shows PE dose-dependent increase in coactivator recruitment to the human SF-1 in vitro. A) PE-18:3 (50 μM 1,2-dilinolenoyl-sn-glycero-3-phosphoethanolamine) but not palmitic acid (50 μM) activates wild-type SF-1 to bind NCoA1 as measured by AlphaScreen. B) Dose-dependent NCoA1 recruitment to SF-1 by PE-18:3. Error bars indicate the standard deviations. The graphs shown are representative of three experiments.

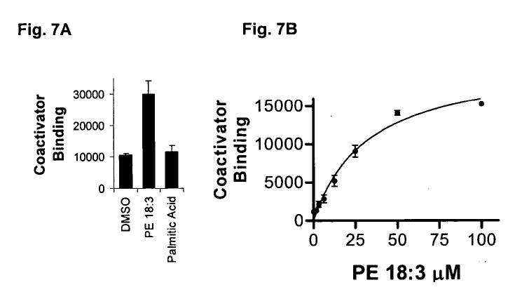

FIGS. 8A-8D shows the effects of pocket residue mutations on human SF-1 and LRH-1 functions in HEK293T cells. A) LRH-1 LBD activity tested as GAL4-DBD fusions acting at a GAL4-responsive LUC reporter gene. The mutations tested include residues A303, L378, A467, Y470 and K474. B) SF-1 LBD activity tested as GAL4-DBD fusions. The mutations tested include residues A269, G341, L344, A433, Y436, and K440. C) Western blot analysis of cells after transfection with vectors encoding GAL4-DBD-LRH-1-LBD fusion proteins using anti-GAL4-DBD antibody. D) Western blot analysis of GAL4-DBD-SF-1-LBD fusion proteins. Error bars indicate the standard deviations. The graphs shown are representative of three independent experiments

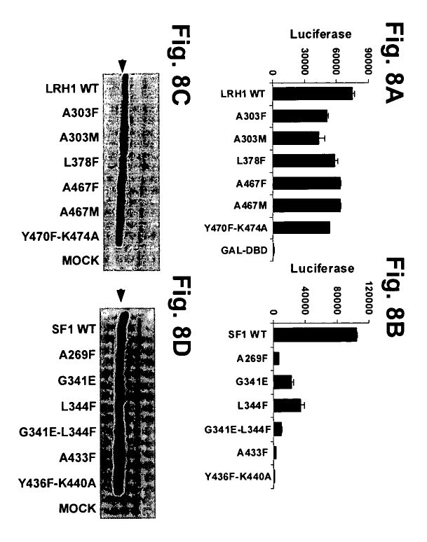

### DETAILED DESCRIPTION OF THE INVENTION

Table 1 provides crystal properties for SF-1 and LRH-1 determined as described in the Examples.

Table 2 provides atomic coordinates for SF1 ligand binding domain polypeptide crystal co-crystallized with a phospholipid ligand as described herein. In this table, the various columns have the following content, beginning with the left-most column:
ATOM: Refers to the relevant moiety for the table row.
Atom number: Refers to the arbitrary atom number designation within the coordinate table.
Atom Name: Identifier for the atom present at the particular coordinates.
Residue Name: Identifier for the residue of the atom for the table row.
Chain ID: Chain ID refers to one monomer of the protein in the crystal, e.g., chain “A”, or to other compound present in the crystal, e.g., HOH for water, and L for a ligand or binding compound. Multiple copies of the protein monomers will have different chain Ids.
Residue Number: The amino acid residue number in the chain.
X, Y, Z: Respectively are the X, Y, and Z coordinate values.
Occupancy: Describes the fraction of time the atom is observed in the crystal. For example, occupancy=1 means that the atom is present all the time; occupancy=0.5 indicates that the atom is present in the location 50% of the time.
B-factor: A measure of the thermal motion of the atom.
Element: Identifier for the element.

Table 3 provides atomic coordinates for LRH1 ligand binding domain polypeptide crystal co-crystallized with a phospholipid ligand as described herein. Table entries are as in Table 2.

Table 4 provides the reference nucleotide sequence for human SF-1 cDNA and the amino acid sequence of the encoded SF-1 polypeptide.

Table 5 provides the reference nucleotide sequence for human LRH-1 cDNA isoform 2 and the corresponding amino acid sequence of the encoded LRH-1 polypeptide, and the reference nucleotide sequence for human LRH-1 cDNA isoform 1 and the encoded amino sequence of the corresponding LRH-1 polypeptide. Additionally, Table 5 provides the nucleotide sequence of mouse LRH-1.

I. General

Steroidogenic factor-1 (SF-1, ADFBP, ELP, NR5A1) and liver receptor homologue-1 (LRH-1, FTF, HB1F, CPF, NR5A2) are ‘orphan’ members of the nuclear receptor family for which no natural ligands have been identified (Fayard et al., Trends Cell Biol., 2004, 14, 250-60; Val et al., Nucl Recept. 2003, 1, 8. These two factors are related to fushi tarazu factor-1 (FTZ-F1) of Drosophila, and comprise the NR5A branch of the nuclear receptor gene family in man. Functional similarities follow their sequence similarities, as SF-1 and LRH-1 both function as monomers (Li et al., J. Biol. Chem., 1998, 273:29022-29031) to regulate genes at similar response elements.

However, SF-1 is expressed predominantly in the adrenals, testis, ventromedial hypothalamus, and pituitary, and regulates genes coordinating adrenal and sex steroid syntheses (Val et al., Nucl. Recept., 2003, 1:8), while LRH-1 is expressed in liver, intestine, and pancreas, and act on genes coordinating bile acid synthesis, enterohepatic circulation, and absorption. (Fayard et al., Trends Cell Biol., 2004, 14:250-260.) Gene knockout and heterozygous loss-of-function studies show that both SF-1 and LRH-1 are essential during embryogenesis for normal development of the organs in which they are expressed and mammalian cell transfection experiments indicate that SF-1 and LRH-1 function as obligate factors for their target genes, acting apparently constitutively. (Pare et al., J. Biol. Chem., 2004, 279, 21206-21216; Zhao et al., Mol. Cell Endocrinol., 2001, 185:27-32; Sadovsky et al., Proc. Natl. Acad. Sci. USA, 1995, 92:10939-10943; Shinoda et al., Dev. Dyn., 1995, 204:22-29; Luo et al., Cell, 1994, 77:481-490; Achermann et al., J. Clin. Endocrinol Metab., 2002, 87:1829-1833.) The mouse LRH-1 structure contains a cavity available for potential ligands, but mutations to fill this cavity did not diminish activity, supporting a model of constitutive, ligand-independent function. (Sablin et al., Mol. Cell, 2003, 11:1575-1585.)

X-ray structures of the ligand-binding domains of human SF-1 and human LRH-1 have been determined. Additionally, it has been discovered that each structure includes a phospholipid ligand. The receptor-ligand interactions indicate that as a class, phospholipids are well-suited as ligands to stabilize the active conformation, a conclusion supported by specific structure-guided mutational analyses. Coactivator-derived peptides included in the co-crystallization experiments bind not only to the canonical activation-function (AF-2) surface of both SF-1 and LRH-1, but in the case of the LRH-1, also to a novel second site. These structures indicate a link between phospholipids and cholesterol regulation, and further, introduce possible new modes of co-regulator recruitment unique to the NR5A branch of the nuclear receptor superfamily.

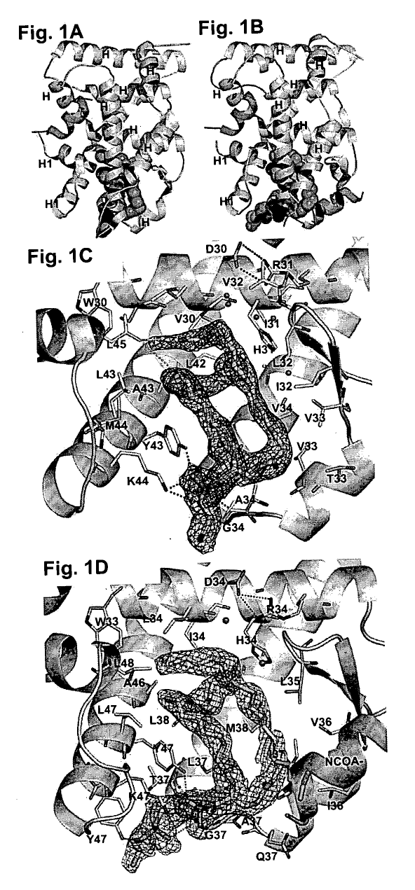

The SF-1 and LRH-1 LBD structures adopt an α-helical sandwich architecture composed of 12 α-helices and one β-hairpin (FIGS. 1A and 1B; Table 1). This protein fold is prototypical of the nuclear receptor superfamily, enclosing a cavity surrounded by several helices and the β-hairpin. (Wurtz et al., Nat. Struct. Biol., 1996, 3, 87-94; Wagner et al., Nature, 1995, 378:690-7.) As observed in mouse LRH-1 (Sablin et al., Mol. Cell, 2003, 11:1575-85.), both the human SF-1 and LRH-1 structures contain a H2 that forms an additional sandwich layer unique to the NR5A family, following a path across and outside of H3 (FIG. 1). This outside path creates an opening to the pocket through a channel formed by H3, H6, H11, and the β-hairpin.

In the SF-1 crystal there are two molecules in the crystallographic asymmetric unit, each delineating residues P221 through K459, one completely and the other incompletely, lacking residues Q249 through R255 in the flexible loop after H2. In the LRH-1 crystal there is one molecule in the asymmetric unit, delineating residues A253 through Q284 and K292 through A492, but also lacking residues 285-291 in the loop after H2. Consistent with reports that SF-1 and LRH-1 function as monomers, none of the crystallization contacts form through the canonical H10 dimerization surface used by other NRs. (Gampe et al., 2000, Mol Cell 5, 545-55; Bourguet et al., 2000, Mol Cell 5, 289-98.)

Strikingly, as indicated above, both structures reveal buried phospholipid molecules derived from the E. coli expression host. Based on well-defined electron density, the molecule in SF-1 can be identified as a phosphatidylethanolamine, and in LRH-1, as a phosphatidylglycerol-phosphoglycerol. In each structure the two acyl chains consist of a palmitic acid (16:0) attached to C1 and apalmitoleic acid (16:1,Δ9) to C2 of the glycerol backbone. The Δ9-cis unsaturation of the palmitoleic acid causes a bend that allows the lipid tails to compact around each other. The polar headgroups of the bound phospholipids reach outside the pocket through the channel formed by H3, H6, H11, and the β-hairpin. In the SF-1 structure the ethanolamine interacts through water molecules to E445 in the loop between H11 and H12. In the LRH-1 structure the glycerol-phosphoglycerol headgroup wraps between the N-terminal end of H7 and the C-terminal end of H11, with the glycerol and phosphate oxygen atoms forming hydrogen bonds with A366 and T377 (H7) and Y473 (H11).

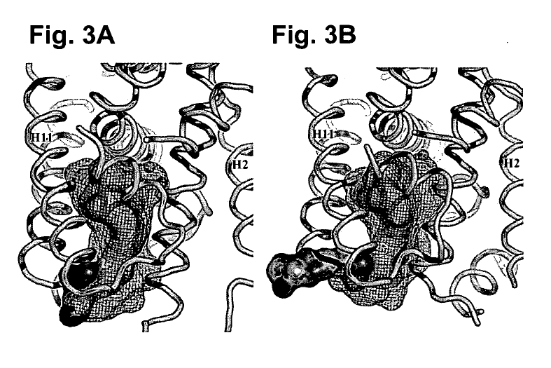

Ligands derived from the expression host have been observed previously in other orphan nuclear receptor structures. In some cases the ligand appears to fill the ligand-binding pocket, making multiple interactions with the protein, suggesting biological relevance. (Kallen et al., 2002, Structure (Camb) 10, 1697-707; Dhe-Paganon et al., 2002, J. Biol. Chem. 277, 37973-6; Wisely et al., 2002, Structure (Camb) 10, 1225-34.) In other cases the ligand is loosely-fit, making interactions with nonconserved residues within the pocket, suggesting these as possible pseudo-ligands. (Stehlin et al., 2001, Embo J. 20, 5822-31.) Phosphatidylethanolamine has also been observed in the structures of the insect nuclear receptor, ultraspiracle, adopting the inactive conformation. (Clayton et al., 2001, Proc Natl Acad Sci USA 98, 1549-54; Billas et al., 2003, Nature 426, 91-6.) The lipids extracted from SF-1 and LRH-1 proteins used here contain several mass spectral peaks that can be interpreted as phosphatidylethanolamine and phosphatidylglycerol, with acyl chain lengths varying from 14 to 18, and of varying saturation. However, the glycerolipid tails of the ligands observed in both the SF-1 and LRH-1 crystal structures are the same, and make extensive van der Waals contacts with hydrophobic residues lining the inside wall of the pocket (FIGS. 1C,D and 2A,B), stabilizing these proteins in the active conformation directly though contacts with the C-terminal activation helix, H12, as well as through hydrophobic interactions with H3 and H 11 that support H12. The total volumes of the LRH-1 and SF-1 cavities are 510 and 550 Å³ respectively (FIG. 3A,B), and with the exception of a polar corner (˜25 Å³ ) that the ligand does not enter, most of the remaining cavity volumes are occupied by the phospholipid ligands.

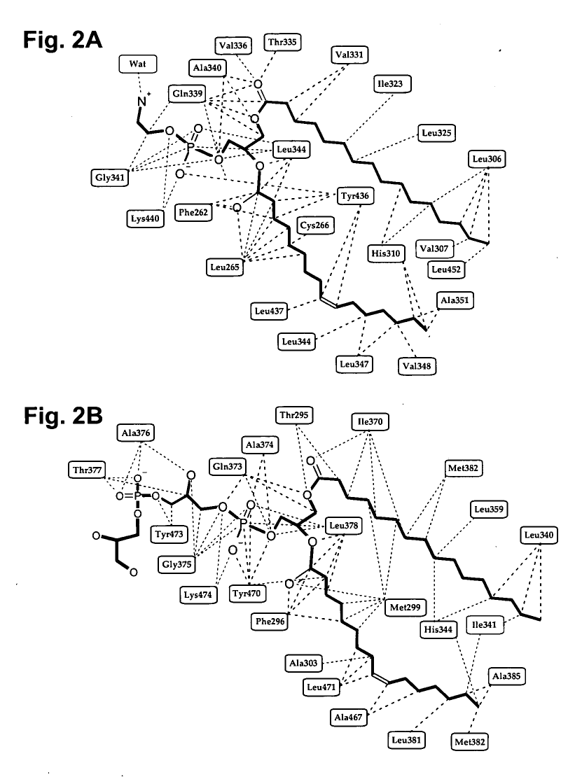

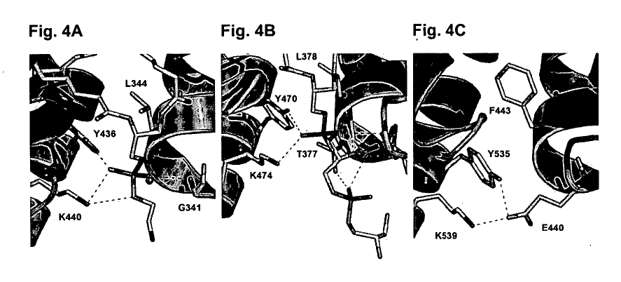

Both SF-1 and LRH-1 make interactions with the phosphate group of the phospholipid that appear likely to affect both ligand affinity and selectivity, and receptor activation. The phosphate lies partially buried, stabilized by forming a salt bridge with a Lys from H11 (K440 in SF-1; K474 in LRH-1), and a hydrogen bond with a Tyr from H 11 (Y436 in SF-1; Y470 in LRH-1) (FIG. 2A,B). The phosphate also makes a hydrogen bond with the backbone amide nitrogen of a Gly from H6 (G341 in SF-1; G375 in LRH-1), thus serving to nucleate the C-terminal ends of H6 and H11 and close off the pocket (FIG. 4A, left and middle). This specific phosphate-binding triad of residues, together with the pocket residues contacting the lipid tails, are highly conserved comparing human LRH-1 and human SF-1, with nineteen of the twenty-two residues identical (FIG. 4B, asterisks). This conservation extends to other species, with seventeen of the twenty-two residues identical comparing the sequences of SF-1 from human, mouse, kangaroo, chicken, turtle, and frog, and LRH-1 from human, chicken, and frog (FIG. 4B), suggesting that SF-1 and LRH-1 from these species recognize similar ligands, and supporting a role for phospholipids as a relevant class of ligand.

Curiously, in the mouse LRH-1 sequence a Glu (residue 440 in mouse) replaces the Gly of the phosphate-binding triad of human LRH-1. In the structure of the mouse LRH-1 this Glu mimics the nucleating interactions with the Lys and Tyr of H11 that the phospholipid phosphorous group makes in other structures of human LRH-1 and SF-1 (FIG. 4C). Just inside the pocket of the human structures a conserved Leu (L344 in SF-1; L378 in LRH-1) exists as Phe in mouse LRH-1 (F443 in mouse LRH-1), helping to bring the N-terminal end of H3 close to H6 and H11 (FIG. 4B,C). Together these two residue changes in the mouse LRH-1 appear to maintain the pocket in a more closed conformation, less able to recognize phospholipid ligands. Of the seventeen residues identical comparing most of the branches of SF-1 and LRH-1, three are changed in the mouse, suggesting mouse is an outlier in its mode of ligand recognition (FIG. 4D). Regulation of bile metabolism differs in man and rodents, that can be partly explained by differences in regulation of CYP7A by the liver-X receptor; the structural differences between mouse and human LRH-1 may also contribute to the species differences. (Goodwin et al., 2003, Mol Endocrinol 17, 386-94.)

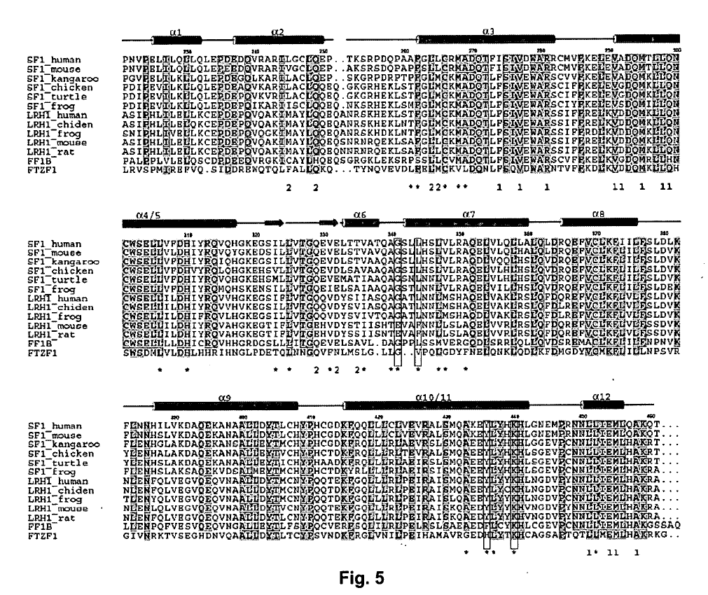

When tested for coactivator binding in vitro, both SF-1 and LRH-1 proteins made in E. coli demonstrated constitutive activity for coactivator recruitment. Addition of phospholipids to these preparations showed little increase in signal, consistent with the preexisting binding of phospholipids. However, the lipids binding SF-1 could be partially extracted by washing the proteins with liposomes prepared using phosphatidylcholine (C22 acyl chain length). It was reasoned that such liposomes with long acyl chains could act as a sink for extracted lipids, without binding the receptors themselves. After such washing the coactivator binding by SF-1 was diminished, but could be activated by the addition of phosphatidylethanolamine (FIG. 5A). The PE 16:0 16:1 observed in the crystal structure is unavailable commercially, so it could not be readily obtained. However PE 18:3 18:3 gave a dose-dependent increase in binding of SRC1. The calculated EC₅₀ in this experiment was 30 μM, comparable to that reported for association of bile acids to their cognate nuclear receptor, FXR. (Parks et al., Science, 1999, 284:1365-8; Makishima et al., Science, 1999, 284:1362-5.)

A selection of structure-guided mutations of SF-1 and LRH-1 pockets were constructed (FIG. 5B) to test their effects on function of these receptors in transfected mammalian cells. When the SF-1 or LRH-1 LBDs were fused to the DNA-binding domain (DBD) of GAL4, strong activation in transfected cells of a reporter gene containing GAL4-responsive elements was observed (FIG. 5C). Mutations of the SF-1 ligand binding pocket, including A269F, G341E, L344F, G341E/L344F and A433F, diminished this activity 68-97% (FIG. 5C) indicating that ligands likely are required for full activation of human SF-1. Mutations of the phosphate-binding residues Y436 and K440 in SF-1 showed the most dramatic lowering effect on activity (99%, FIG. 5C), which is the most suggestive that phospholipids likely act as ligands for SF-1. These mutations are located in the channel to the pocket, and therefore would not interfere with ligands that bind more deeply in the pocket.

Six pocket mutations, A303F, A303M, L378F, A467F, A467M, and Y470F/K474A were tested in LRH-1 (FIG. 5D), and found to diminish activity 16-42% (FIG. 5D), indicating that ligands are likely also required for full activation of human LRH-1. However the equivalent mutations were weaker comparing human LRH-1 and SF-1, suggesting human LRH-1 has a more pronounced apparent constitutive activity, as observed with the mouse LRH-1. The pocket mutants of SF-1 were not observed to alter the expression or stability of these LBDs when tested in E. coli; the expression of each was the same as WT (˜20 mg per liter culture). These data indicate that SF-1 and LRH-1 do not require ligands as constitutive structural cofactors, as has been suggested for another nuclear receptor, HNF4, but rather behave as expected for ligand-regulated receptors.

Both the SF-1 and LRH-1 structures were obtained as complexes with a peptide matching the NR-box 3 of the coactivator NCOA2 (TIF2). The coactivator peptide bound the canonical AF-2 surface through specific sidechain interactions (FIG. 1A,B). (Feng et al., Science, 1998, 280:1747-9; Nolte et al., Nature, 1998, 395:137-43; Marimuthu et al., Mol. Endocrinol., 2002, 16:271-86.) H12 adopts the active AF-2 conformation, and hydrophobic residues from H3 (SF-1: F273, 1274, V277 and LRH-1: L307, F308, V311), H4 (SF-1: V291, M295, L298 and LRH-1:V325, M329, L332 ), and H12 (SF-1: L451, M455 and LRH-1: L485, M489), form a grooved binding surface complementary to the hydrophobic LXXLL motif of NCOA2. Charged residues from H3 (SF-1:R281 and LRH-1: R315) and H12 (SF-1:E454 and LRH-1: E488) form a charge-clamp with the bound peptide backbone. In other crystallization experiments, a synthetic peptide matching the NR-box 2 peptide from another coactivator NCOA1 (SRC-1) was co-crystallized with the SF-1, and found to interact with the same surface.

Surprisingly, in the LRH-1 structure a coactivator peptide was also bound to a novel second site on the surface formed by residues of H2 (M277, L280), H3 (T295, L298, M299, and M302), the β-hairpin (V365), and H6 (1369) that form a hydrophobic patch complementary to the LRYLL motif of the peptide. The hydrophobic patch also includes atoms of the C1 acyl chain of the phospholipid, in coordination with the methyl group of T295, suggesting a direct participation by the ligand in recruitment of coactivator to this site. Unlike the canonical binding site, there is no strong charge-clamp to the coactivator peptide dipole in the second binding site. However the Tyr of the peptide forms a hydrogen bond with D366 of the β-hairpin, suggesting the residue at the second X of the LXXLL motif will influence the coactivator selectivity. Although no second peptide was bound in the SF-1 crystal, the surface features of SF-1 are similar enough with LRH-1 to suggest that SF-1 could also bind coactivators at this site. The difference in results may be due to crystal packing differences; in the LRH-1 crystal the second peptide is located at a favorable crystal packing interface, but in the SF-1 crystal the packing interferes with peptide binding to this site.

Mutated forms of LRH-1 were engineered for analysis of the novel second coactivator binding site observed in the structure (FIG. 5E,F). Binding of coactivator fragments to LRH-1 is strong enough to observe easily through co-expression of the two proteins in E. coli, followed by metal affinity purification of the His-tagged LRH-1 (FIG. 5G,H)). Compared to the LRH-1-WT protein, a mutation of the canonical coactivator site, E488K, caused 70% decrease in coactivator fragment binding (FIG. 5G). However, secondary mutations of the residues that define the novel coactivator-binding surface (D366A, and 1369Y) blocked the remainder of the binding (FIG. 5H). When tested singly, the mutations of the second site were weaker than the mutation of the canonical site in lowering coactivator binding (FIG. 5G). The coactivator site mutants of SF-1 and LRH-1 LBDs were tested as GAL4 DBD fusions in mammalian transfection experiments, with results supporting a functional participation of the novel site to recruit coactivators.

In LRH-1 mutation of the canonical site gave strong reductions in activity (96%), suggesting that under these conditions the canonical site is dominant (FIG. 5I). However mutations of the novel site, M277K and D366A, also lowered activity (40%, FIG. 5I). In SF-1 mutation of the canonical site gave a partial lowering (48%, FIG. 5J); mutations of the novel site, L245K and E332A, gave similar reductions in activity (50% and 41%, FIG. 5J), suggesting a secondary coactivator-binding site also functions on SF-1. It has been reported that some co-regulators, including DAX1 and PROX1, are relatively independent of the canonical coactivator site on the NR5A sub-family. (Marimuthu et al., 2002, Mol Endocrinol 16, 271-86; Crawford et al., Mol. Endocrinol., 1997, 11:1626-35; Suzuki et al., Mol. Cell Biol., 2003, 23:238-49; Qin et al., Mol Endocrinol., 2004, 18:2424-2439.) This novel second site may be a site of binding inferred by these studies. Thus, the NR5A subfamily, functioning as monomers, may require two coactivator-binding sites, compared to other NRs that function as homo- or hetero-dimers, requiring one each. Alternatively, the two sites may bind independently to two coregulators, thereby integrating multiple signals.

In addition to the structural and functional analysis indicated above, phospholipids as ligands for SF-1 and LRH-1 is also reasonable based on mechanistic rationale. Both receptors regulate genes important for cholesterol metabolism. Phospholipid composition must be balanced with cholesterol content in membranes to maintain proper membrane fluidity, and therefore regulation of genes for cholesterol metabolism by a phospholipid signal makes sense. (McConnell & Radhakrishnan, Biochim Biophys Acta 2003, 1610:159-73; Quinn, Prog. Biophys. Mol. Biol., 1981, 38:1-104.) This may be especially true for cells of the adrenal and liver that are specialized for high flux and turnover of cholesterol. (Jefcoate, J. Clin Invest., 2002, 110:881-90.) In fact, a major source of phospholipid in such cells derives from the blood lipoprotein particles, that are known to carry large amounts of phospholipid in addition to cholesterol, so a source of phospholipid signals may derive from these particles. (Vance & Vance, J. Biol. Chem., 1986, 261:4486-91; Wang et al., J. Biol. Chem., 2003, 278:42906-12.) Whether derived from the blood or from intracellular synthesis, phospholipid composition is known to vary with nutrition, exercise, pregnancy, and other metabolic and hormonal status, and such changes could lead to variable NR5A activation, or conceivably, inhibition. (Clamp et al., Lipids, 1997, 32:179-84; Tranquilli et al., Acta Obstet. Gynec., Scand., 2004, 83:443-8; Imai et al., Biochem. Pharmacol., 1999, 58:925-33; Lin et al., J. Lipid Res., 2004, 45:529-35; Andersson et al., Am. J Physiol., 1998, 274:E432-8.) Therefore ligand regulation of these receptors should be considered within a general context of lipid homeostasis. It is noteworthy that cholesterol and phosphatidylethanolamine have been documented to regulate, in mammals and insects respectively, the post-translational processing of the nuclear factor, SREBP, that is important in the regulation of many genes of lipid homeostasis, in some cases cooperating with SF-1. (Wang et al., Cell, 1994, 77:53-62; Dobrosotskaya et al., Science, 2002, 296:879-83; Lopez & McLean, Endocrinology, 1999, 140:5669-81.) Thus the identification of phospholipid as a class of molecule regulating SF-1 and LRH-1, provided by the current X-ray structures provides target structures and allows the identification and development of modulators of these receptors.

II. Applications of SF1 and LRH1 Modulators and Exemplary Assay Methods

A. LRH-1

Compounds that modulate LRH-1 activity can have beneficial effects in the management of cholesterol excess. Thus, activators of LRH-1 would lower circulating cholesterol levels. This is because LRH-1 regulates several genes involved in cholesterol homeostasis, including: CYP7A1, the rate-limiting enzyme for conversion of cholesterol to bile acids (Wang et al., J. Lipid Res., 1996, 37:1831-41; Nitta et al., Proc. Natl. Acad. Sci. USA, 1999, 96:6660-5), the scavenger receptor class B type I (SR-BI), that mediates selective cellular cholesterol uptake from high-density lipoproteins (HDLs) (Schoonjans et al., EMBO Rep., 2002, 3:1181-7), and cholesterol ester transfer protein (CETP), important for remodeling of HDL particles (Luo et al., J. Biol. Chem. , 2001, 276:24767-73).

A second indication for LRH-1 modulators is in treatment or management of hepatitis virus infection. Hepatitis B virus is the major cause of acute and chronic hepatitis, and is associated with development of hepatocellular carcinoma. Certain hepatitus virus genes are stimulated by LRH-1. (Li et al., J. Biol. Chem., 1998, 273, 29022-31; (Gilbert et al., J. Virol., 2000, 74, 5032-9.) Thus inhibitors or modulators of LRH-1 would limit the functions of the hepatitis virus, with beneficial effects on infected individuals.

LRH-1 also regulates other genes important for cholesterol homeostasis, including:
Apical sodium-dependent bile acid transporter (ASBT), important for bile acid recycling (Chen, F., et al., J. Biol. Chem., 2003. 278:19909-19916);
Sterol 12alpha-hydroxylase (CYP8B), involved in synthesis of the more polar bile acids, such as cholic acid (del Castillo-Olivares, A. & G. Gil, J. Biol. Chem., 2000. 275:17793-17799);
Scavenger receptor class B type I (SR-BI), mediates selective cellular cholesterol uptake from high-density lipoproteins (HDLs), important in the reverse cholesterol transport process (Schoonjans, K., et al., EMBO Rep, 2002., 3:1181-1187);
Alpha-fetoprotein, an early marker of fetal liver development, and steroid-binding protein (Galarneau, L., et al., Mol Cell Biol, 1996., 16:3853-3865);
Cholesterol ester transfer protein (CETP), involved in reverse cholesterol transport, and in remodeling of HDL particles (Luo, Y., et al., J. Biol. Chem., 2001, 276:24767-24773);
Carboxyl ester lipase (CEL), made in the pancreas, important for hydrolysis of dietary cholesterol esters (Fayard, E., et al., J. Biol. Chem., 2003, 278:35725-35731);
Multidrug resistance protein (MRP3), a transporter that likely functions to export bile salts from hepatocytes and enterocytes (Inokuchi, A., et al., J. Biol. Chem., 2001, 276:46822-46829);
Short heterodimer partner (SHP), a protein that regulates LRH-1 and other nuclear receptors (Lee, Y. K., et al., J. Biol. Chem., 1999, 274:20869-20873.)

Other targets of LRH-1 include:
Hepatocyte nuclear factor 4 alpha (HNF4α), a nuclear receptor important in regulation by fatty acids. Also, HNF3β and HNF1α two other liver-specific transcription regulators (Pare, J. F., et al., J. Biol. Chem., 2001, 276:13136-13144);
Aromatase cytochrome P450 (CYP19), that catalyzes estrogen syntheseis in adipose tissue, and may contribute to the severity of breast cancer. (Clyne, C. D., et al., J. Biol. Chem., 2002, 277:20591-20597.)

Thus, such additional LRH-1 targets can also be used for assaying or screening for modulators of LRH-1. Such modulators can then be used for treatment of diseases or conditions associated with those additional LRH-1 target genes.

B. SF-1

Compounds that modulate SF-1 can have desireable effects on sexual function and sex-related phenotypic aspects. SF-1 is very important during prenatal development of the sexual anatomy. In conjunction with a genetic screening protocol, in situations that are expected to lead to phenotypic development unsupportive of the primary sexual genotype could be corrected, at least in part, by modulation of SF-1.

SF-1 also functions after birth to regulate genes involved in sex hormone synthesis in the testis or ovaries. Thus modulation of SF-1 should assist in the maintenance of sexual function or of sex-related phenotypic appearance.

SF-1 also regulates genes important for the synthesis of adrenal steroids. Thus it controls the levels of a set of very potent hormone regulators of lipid and carbohydrate metabolism (glucocorticoids), and hypertension (mineralocorticoids). SF-1 is a key regulator in the hypothalamic-pituitary-adrenal axis through which environmental factors such as stress, or physiological factors such as starvation, have effects on overall physiology and metabolism. Pharmaceutical modulators of SF-1 can assist in maintaining a normal physiological balance in situations where the unassisted organs are over-reacting to environmental effects (such as too much stress) or medical procedures (such as surgery or other interventional procedures), or drug-induced manipulations intended to intervene in a subset of the normal metabolic regulatory mechanisms.

Pharmaceutical modulators of SF-1 can also be used in the management of ectopic tumors that produce steroid hormones. Initially modulators of SF-1 can be useful in the diagnosis of abnormal steroid production. Once a diagnosis of steroid-producing tumors is established but before surgical procedures are implemented, normal (or closer to normal) physiological tone can be produced with inhibitors of SF-1. In the case of brain or other tumor locations or conditions in which surgery is difficult, longer-term treatment with SF-1 modulators would be valuable.

Modulators of SF-1 would also be useful for treatment of conditions of poisoning with endocrine-disrupting agents, such as pesticides and polychlorinated biphenyls (PCBs), known to interfere with normal endocrine function. But certainly these agents interfere with the normal production of hormones regulated by SF-1 function, and some may interfere directly with SF-1 function. Thus modulators of SF-1 can reverse the negative effects by such compounds.

SF-1 regulates most of the genes encoding enzymes catalyzing the synthesis of steroid hormones, including P450 cholesterol sidechain cleavage enzyme (CYP11A1) (Hu, M. C., et al., Mol. Endocrinol., 2001, 15:812-818), 11-b-hydroxylase (CYP11B1), aldosterone synthase (CYP11B2), CYP17, CYP19; see, e.g., Mascaro, C., et al., Biochem J., 2000. 350 (Pt 3):785-790, for review.

SF-1 also regulates the gene encoding steroidogenic acute regulatory (StAR) protein, that transports cholesterol into the mitochondria where steroids are synthesized. This transport is the rate-limiting step for steroidogenesis.

Other target genes of SF-1 include, for example:
3-hydroxy-3-methylglutaryl-CoA (HMG-CoA) synthase, that catalyses an early step in the synthesis of cholesterol (Mascaro, C., et al., Biochem .J, 2000. 350 (Pt 3):785-790);
Scavenger receptor class B type I (SR-BI), mediates selective cellular cholesterol uptake from high-density lipoproteins (HDLs), important in the reverse cholesterol transport process (Lopez et al., Endocrinology, 1999, 140:3034-3044; Cao, G., et al., J. Biol. Chem., 1997. 272: 33068-33076);
Sterol carrier protein-2 (SCP-2) that mediates intracellular cholesterol transport in steroidogenic tissues;
Adrenocorticotropin receptor, that transmits the signal to activate adrenal steroidogenesis from the pituitary hormone, adrenocorticotropin;
Follicle stimulating hormone and leutinizing hormone receptors, that transmits the signal to activate the gonadal steroidogenesis from the pituitary hormone.

Thus, such additional SF-1 targets can also be used for assaying or screening for modulators of SF-1. Such modulators can then be used for treatment of diseases or conditions associated with those additional SF-1 target genes.

Nuclear receptors that are highly structurally related to SF-1 are present in most insects, as SF-1 (and LRH-1) comprise the members of the nuclear receptors in man that are most related to the FTZ-F1 receptors in insects. Thus, modulators of SF-1 could serve as effective insecticides through actions on an insect receptor related to SF-1, or as molecular scaffolds or reference compounds for developing effective insecticides. Such development can be carried out as described herein for development of modulators of SF-1 and LRH-1 using the respective insect FTZ-F1 receptor, or by using conventional medicinal chemistry to select and test derivatives of the SF-1 or LRH-1 active compounds.

For example, sequence alignments of all 48 human nuclear receptors indicate that SF-1 and LRH-1 are highly related: these receptors are within the NR5 subfamily of the nuclear receptor (NR) superfamily. When the SF-1 and LRH-1 sequences are compared to all currently known sequences from all species, it is observed that the NR5 subfamily also includes the FTZ-F1 gene from Drosophila. Because Drosophila is a member of the Insect class of eukaryotes, it is likely that inhibitors of SF-1 and LRH-1 as provided herein will have insecticidal properties or inhibit insect development. Thus, compounds provided by the present invention can be used to target many diverse insect pests such as flies, gnats, and fleas among many other types. Furthermore, compounds provided by the invention that bind to SF-1 and LRH-1 can be used to refine other compounds that bind to FTZ-F1. Also, the crystal structures of SF-1 and LRH-1 provided by the invention can be used to make models of FTZ-F1 to predict how one or a series of potential ligands for FTZ-F1 will bind to that target; thereby facilitating development of FTZ-F1-inhibiting compounds.

Screening for molecules, e.g., small molecules, that bind to and modulate the SF-1 and LRH-1 receptors can be accomplished using in vitro assays that quantify the amount of binding of co-regulatory proteins with the SF-1 or LRH-1 receptor proteins. Several co-regulatory proteins have been documented to bind to these receptors, including SRC-1, TReP-132, DAX-1, and SHP. The receptor proteins can be produced in E. coli or other convenient expression system. The co-regulatory proteins are typically too large to be conveniently made as full-length proteins; however the relevant receptor-binding motifs can be produced in E. coli. Alternatively, peptides can be chemically synthesized that contain these co-regulator motifs and used in the assays.

A variety of different methods for detecting molecular interactions can be used. For example, Alpha Screen technology (Perkin Elmer) is suitable to detect the interaction of the receptor with the coactivator fragment. In this case it is suitable to engineer the ligand-binding domain of the SF-1 and LRH-1 as an N-terminally HIS-tagged protein that can bind the acceptor bead (containing Nickel moieties that will bind the HIS tag). The coactivator fragment can be synthesized containing a biotin moiety that will bind the donor bead. In the presence of ‘activating’ compounds the association of the receptor with the co-regulator may be strengthened, whereas the presence of ‘inhibitory’ compounds may destabilize this interaction. Libraries of chemicals, or derivatives, can be quantified for their effects on co-regulator binding.

Thus, in an exemplary implementation, the Alpha Screen Histidine detection (Nickel chelate) kit (Perkin Elmer) is used to detect binding between His-tagged receptor LBD and biotinylated coactivator peptides or fragments. The assay is performed in Costar 384-well white polystyrene plates (Coming Inc.), in a total volume of 20 μL. Compounds to be tested for their abilities to modulate the interaction of nuclear receptor with coactivator are added to the 384-well plate in 1 μL of DMSO or buffer in advance of addition of the receptor and coactivator proteins.

Reactions are initiated in 15 μl containing 50 nM His-tagged nuclear receptor and 50 mM biotin-tagged coactivator fragment, using buffer containing 50 mM Bis-tris HCl (pH 7.0), 50 mM KCl, 0.05% Tween 20, 1 mM DTT, 0.1% BSA. Other buffer variations can be tested to optimize the largest difference in signals obtained using the apo receptor and receptor bound to compounds already determined to bind and activate the receptor. After the protein solutions are added to the compounds, the plate is sealed and incubated at room temp for 2 hours. After incubation, a 5 μL mixture containing streptavidin donor beads (15 μg/ml) and Ni-chelate acceptor beads (15 μg/ml) are added from the Nickel chelate kit. Plates are resealed and incubated in the dark for 2 hours at room temperature and then read in an AlphaFusion reader set at a read time of 1 s/well.

A signal is produced by the binding of coactivator to nuclear receptor that can be detected by the AlphaFusion reader (the binding brings the acceptor beads into close proximity of the donor beads, which allows the acceptor beads to detect the singlet oxygens produced by the donor beads, causing them to emit a light detected by the instrument). Data analysis can be performed using GraphPad Prism (GraphPad Software, Inc.). The relative abilities of many compounds to activate the receptor can be assessed by calculating and comparing each of their EC₅₀ values (i.e., the concentration of compound that causes 50% of the maximal effect, interpolated from the results of a series of tests with varying concentrations of each compound).

C. Assaying the Effects of Ligands in Cell Culture

Ligands that modulate the interaction of SF-1 or LRH-1 with co-regulators will affect the expression of genes that are targets of these receptors. Thus assays of the levels of expression of these genes will indicate the effect such compounds are having. For SF-1 an exemplary suitable cell type is the H-295R human adrenal cell. This cell expresses the enzymes, transport proteins, and receptors required for steroid hormone synthesis, and in fact makes the steroid hormone, progesterone, in assayable amounts. After treatment with a ligand, the levels of mRNA encoding these proteins can be quantified by QPCR methods. Alternatively the levels of progesterone can be assayed.

In the case of LRH-1, an exemplary suitable cell type is the HepG2 human liver cell. This cell expresses enzymes, receptors, and transporters important for bile acid synthesis. After treatment with a ligand, the levels of mRNA encoding one or more of these proteins can be quantified by QPCR methods as indicators of the effects of LRH-1 modulation.

III. Development of SF-1 and LRH-1 Active Compounds

A. Modulator Identification and Design

A large number of different methods can be used to identify modulators and to design improved modulators. Some useful methods involve structure-based design.

Structure-based modulator design and identification methods are powerful techniques that can involve searches of computer databases containing a wide variety of potential modulators and chemical functional groups. The computerized design and identification of modulators is useful as the computer databases contain more compounds than the chemical libraries, often by an order of magnitude. For reviews of structure-based drug design and identification (see Kuntz et al., Acc. Chem. Res., 1994, 27:117; Guida Current Opinion in Struc. Biol., 1994, 4:777; Cohnan, Current Opinion in Struc. Biol., 1994, 4: 868).

The three dimensional structure of a polypeptide defined by structural coordinates can be utilized by these design methods, for example, the structural coordinates of SF-1 or LRH-1. In addition, the three dimensional structures of SF-1 or LRH-1 determined by the homology, molecular replacement, and NMR techniques can also be applied to modulator design and identification methods.

For identifying modulators, structural information for SF-1 or LRH-1, in particular, structural information for the active site of the SF-1 or LRH-1, can be used. However, it may be advantageous to utilize structural information from one or more co-crystals of the receptor with one or more binding compounds. It can also be advantageous if the binding compound has a structural core in common with test compounds.

1. Design by Searching Molecular Data Bases

One method of rational design searches for modulators by docking the computer representations of compounds from a database of molecules. Publicly available databases include, for example:

a) ACD from Molecular Designs Limited

b) NCI from National Cancer Institute

c) CCDC from Cambridge Crystallographic Data Center

d) CAST from Chemical Abstract Service

e) Derwent from Derwent Information Limited

f) Maybridge from Maybridge Chemical Company LTD

g) Aldrich from Aldrich Chemical Company

h) Directory of Natural Products from Chapman & Hall

One such data base (ACD distributed by Molecular Designs Limited Information Systems) contains compounds that are synthetically derived or are natural products. Methods available to those skilled in the art can convert a data set represented in two dimensions to one represented in three dimensions. These methods can be carried out using such computer programs as CONCORD from Tripos Associates or DE-Converter from Molecular Simulations Limited.

Multiple methods of structure-based modulator design are known to those in the art. (Kuntz et al., J. Mol. Biol., 1982, 162:269; Kuntz et al., Acc. Chem. Res., 1994, 27:117; Meng et al., J. Comp. Chem., 1992, 13: 505; Bohm, J. Comp. Aided Molec. Design, 1994, 8: 623.)

A computer program widely utilized by those skilled in the art of rational modulator design is DOCK from the University of California in San Francisco. The general methods utilized by this computer program and programs like it are described in three applications below. More detailed information regarding some of these techniques can be found in the Accelrys User Guide, 1995 (Accelrys, San Diego, Calif.) A typical computer program used for this purpose can perform a process comprising the following steps or functions:
a) remove the existing compound from the protein;
b) dock the structure of another compound into the active-site using the computer program (such as DOCK) or by interactively moving the compound into the active-site;
c) characterize the space between the compound and the active-site atoms;
d) search libraries for molecular fragments which (i) can fit into the empty space between the compound and the active-site, and (ii) can be linked to the compound; and
e) link the fragments found above to the compound and evaluate the new modified compound.

Part (c) refers to characterizing the geometry and the complementary interactions formed between the atoms of the active site and the compounds. A favorable geometric fit is attained when a significant surface area is shared between the compound and active-site atoms without forming unfavorable steric interactions. One skilled in the art would note that the method can be performed by skipping parts (d) and (e) and screening a database of many compounds.

Structure-based design and identification of modulators of SF-1 and LRH-1 function can be used in conjunction with assay screening. As large computer databases of compounds (around 10,000 compounds) can be searched in a matter of hours or even less, the computer-based method can narrow the compounds tested as potential modulators of SF-1 or LRH-1 function in biochemical or cellular assays.

The above descriptions of structure-based modulator design are not all encompassing and other methods are reported in the literature and can be used, e.g.:
a) CAVEAT: Bartlett et al., in Chemical and Biological Problems in Molecular Recognition, Roberts, S. M.; Ley, S. V.; Campbell, M. M. eds.; Royal Society of Chemistry, 1989, Cambridge, pp. 182-196.
b) FLOG: Miller et al., J. Comp. Aided Molec. Design, 1994, 8:153.
c) PRO Modulator: Clark et al., J. Comp. Aided Molec. Design, 1995, 9:13.
c) MCSS: Miranker and Karplus, Proteins: Structure, Function, and Genetics, 1991, 11:29.
e) AUTODOCK: Goodsell & Olson, Proteins: Structure, Function, and Genetics, 1990, 8:195.
f) GRID: Goodford, J. Med. Chem., 1985, 28:849.

2. Design by Modifying Compounds in Complex with SF-1 and LRH-1

Another way of identifying compounds as potential modulators is to modify an existing modulator in the polypeptide active site. For example, the computer representation of modulators can be modified within the computer representation of a SF-1 or LRH-1 active site (e.g., LBD pocket). betailed instructions for this technique can be found, for example, in the Accelrys User Manual, 1995 in LUDI. The computer representation of the modulator is typically modified by the deletion of a chemical group or groups or by the addition of a chemical group or groups.

Upon each modification to the compound, the atoms of the modified compound and active site can be shifted in conformation and the distance between the modulator and the active-site atoms may be scored along with any complementary interactions formed between the two molecules. Scoring can be complete when a favorable geometric fit and favorable complementary interactions are attained. Compounds that have favorable scores are potential modulators.

3. Design by Modifying the Structure of Compounds that Bind SF-1 or LRH-1

A third method of structure-based modulator design is to screen compounds designed by a modulator building or modulator searching computer program. Examples of these types of programs can be found in the Molecular Simulations Package, Catalyst. Descriptions for using this program are documented in the Molecular Simulations User Guide (1995). Other computer programs used in this application are ISIS/HOST, ISIS/BASE, ISIS/DRAW) from Molecular Designs Limited and UNITY from Tripos Associates.

These programs can be operated on the structure of a compound that has been removed from the active site of the three dimensional structure of a compound-receptor complex. Operating the program on such a compound is preferable since it is in a biologically active conformation.

A modulator construction computer program is a computer program that may be used to replace computer representations of chemical groups in a compound complexed with a receptor or other biomolecule with groups from a computer database. A modulator searching computer program is a computer program that may be used to search computer representations of compounds from a computer data base that have similar three dimensional structures and similar chemical groups as compound bound to a particular biomolecule.

A typical program can operate by using the following general steps:
a) map the compounds by chemical features such as by hydrogen bond donors or acceptors, hydrophobic/lipophilic sites, positively ionizable sites, or negatively ionizable sites;
b) add geometric constraints to the mapped features; and
c) search databases with the model generated in (b).

Those skilled in the art also recognize that not all of the possible chemical features of the compound need be present in the model of (b). One can use any subset of the model to generate different models for data base searches.

B. Identification of Active Compounds Using SF-1 or LRH-1 Structure and Molecular Scaffolds

In addition to the methods described above that are normally applied based on screening hits that have a substantial level of activity, the availability of crystal structures that include ligand binding sites for SF-1 and LRH-1 enables application of a scaffold method for identifying and developing additional active compounds.

Thus, the present invention also concerns methods for designing ligands active on SF-1 or LRH-1 by using structural information about the respective ligand binding sites and identified binding compounds. While such methods can be implemented in many ways (e.g., as described above), advantageously the process utilizes molecular scaffolds. Such development processes and related methods are described generally below, and can, as indicated, be applied to SF-1 and LRH-1, individually or as a family.

Molecular scaffolds as discussed herein are low molecular weight molecules that bind with low or very low affinity to the target and typically have low or very low activity on that target and/or act broadly across families of target molecules. The ability of a scaffold or other compound to act broadly across multiple members of a target family is advantageous in developing ligands. For example, a scaffold or set of scaffolds can serve as starting compounds for developing ligands with desired specificity or with desired cross-activity on a selected subset of members of a target family. Further, identification of a set of scaffolds that each bind with members of a target family provides an advantageous basis for selecting a starting point for ligand development for a particular target or subset of targets. In many cases, the ability of a scaffold to bind to and/or have activity on multiple members of a target family is related to active site or binding site homology that exists across the target family.

A scaffold active across multiple members of the target family interacts with surfaces or residues of relatively high homology, i.e., binds to conserved regions of the binding pockets. Scaffolds that bind with multiple members can be modified to provide greater specificity or to have a particular cross-reactivity, e.g., by exploiting differences between target binding sites to provide specificity, and exploiting similarities to design in cross-reactivities. Adding substituents that provide attractive interactions with the particular target typically increases the binding affinity, often increasing the activity. The various parts of the ligand development process are described in more detail in following sections, but the following describes an advantageous approach for scaffold-based ligand development.

Scaffold-based ligand development (scaffold-based drug discovery) can be implemented in a variety of ways, but large scale expression of protein is useful to provide material for crystallization, co-crystallization, and biochemical screening (e.g., binding and activity assays). For crystallization, crystallization conditions can be established for apo protein and a structure determined from those crystals. For screening, preferably a biased library selected for the particular target family is screened for binding and/or activity on the target. Highly preferably a plurality of members from the target family is screened. Such screening, whether on a single target or on multiple members of a target family provides screening hits. Low affinity and/or low activity hits are selected. Such low affinity hits can either identify a scaffold molecule, or allow identification of a scaffold molecule by analyzing common features between binding molecules. Simpler molecules containing the common features can then be tested to determine if they retain binding and/or activity, thereby allowing identification of a scaffold molecule.

When multiple members of a particular target family are used for screening, the overlap in binding and/or activity of compounds can provide a useful selection for compounds that will be subjected to crystallization. For example, for 3 target molecules from a target family, if each target has about 200-500 hits in screening of a particular library, much smaller subsets of those hits will be common to any 2 of the 3 targets, and a still smaller subset will be common to all 3 targets, e.g., 100-300. In many.cases, compounds in the subset common to all 3 targets will be selected for co-crystallography, as they provide the broadest potential for ligand development.

Once compounds for co-crystallography are selected, conditions for forming co-crystals are determined, allowing determination of co-crystal structure, and the orientation of binding compound in the binding site of the target is determined by solving the structure (this can be highly assisted if an apo protein crystal structure has been determined or if the structure of a close homolog is available for use in a homology model.) Preferably the co-crystals are formed by direct co-crystallization rather than by soaking the compound into crystals of apo protein.

From the co-crystals and knowledge of the structure of the binding compounds, additional selection of scaffolds or other binding compounds can be made by applying selection filters, e.g., for (1) binding mode, (2) multiple sites for substitution, and/or (3) tractable chemistry. A binding mode filter can, for example, be based on the demonstration of a dominant binding mode. That is, a scaffold or compounds of a scaffold group bind with a consistent orientation, preferably a consistent orientation across multiple members of a target family. Filtering scaffolds for multiple sites for substitution provides greater potential for developing ligands for specific targets due to the greater capacity for appropriately modifying the structure of the scaffold. Filtering for tractable chemistry also facilitates preparation of ligands derived from a scaffold because the synthetic paths for making derivative compounds are available. Carrying out such a process of development provides scaffolds, preferably of divergent structure.

In some cases, it may be impractical or undesirable to work with a particular target for some or all of the development process. For example, a particular target may be difficult to express, by easily degraded, or be difficult to crystallize. In these cases, a surrogate target from the target family can be used. It is desirable to have the surrogate be as similar as possible to the desired target, thus a family member that has high homology in the binding site should be used, or the binding site can be modified to be more similar to that of the desired target, or part of the sequence of the desired target can be inserted in the family member replacing the corresponding part of the sequence of the family member.

Once one or more scaffolds are identified for a target family, the scaffolds can be used to develop multiple products directed at specific members of the family, or at specific subsets of family members. Thus, starting from a scaffold that acts on multiple member of the target family, derivative compounds (ligands) can be designed and tested that have increasing selectivity. In addition, such ligands are typically developed to have greater activity, and will also typically have greater binding affinity. In this process, starting with the broadly acting scaffold, ligands are developed that have improved selectivity and activity profiles, leading to identification of lead compounds for drug development, leading to drug candidates, and final drug products.

C. Scaffolds

Typically it is advantageous to select scaffolds (and/or compound sets or libraries for scaffold or binding compound identification) with particular types of characteristics, e.g., to select compounds that are more likely to bind to a particular target and/or to select compounds that have physical and/or synthetic properties to simplify preparation of derivatives, to be drug-like, and/or to provide convenient sites and chemistry for modification or synthesis.

Useful chemical properties of molecular scaffolds can include one or more of the following characteristics, but are not limited thereto: an average molecular weight below about 350 daltons, or between from about 150 to about 350 daltons, or from about 150 to about 300 daltons; having a clogP below 3; a number of rotatable bonds of less than 4; a number of hydrogen bond donors and acceptors below 5 or below 4; a Polar Surface Area of less than 100 Å².; binding at protein binding sites in an orientation so that chemical substituents from a combinatorial library that are attached to the scaffold can be projected into pockets in the protein binding site; and possessing chemically tractable structures at its substituent attachment points that can be modified, thereby enabling rapid library construction.

The term “Molecular Polar Surface Area (PSA)” refers to the sum of surface contributions of polar atoms (usually oxygens, nitrogens and attached hydrogens) in a molecule. The polar surface area has been shown to correlate well with drug transport properties, such as intestinal absorption, or blood-brain barrier penetration.

Additional useful chemical properties of distinct compounds for inclusion in a combinatorial library include the ability to attach chemical moieties to the compound that will not interfere with binding of the compound to at least one protein of interest, and that will impart desirable properties to the library members, for example, causing the library members to be actively transported to cells and/or organs of interest, or the ability to attach to a device such as a chromatography column (e.g., a streptavidin column through a molecule such as biotin) for uses such as tissue and proteomics profiling purposes.

A person of ordinary skill in the art will realize other properties that can be desirable for the scaffold or library members to have depending on the particular requirements of the use, and that compounds with these properties can also be sought and identified in like manner. Methods of selecting compounds for assay are known to those of ordinary skill in the art, for example, methods and compounds described in U.S. Pat. Nos. 6,288,234, 6,090,912, and 5,840,485, each of which is hereby incorporated by reference in its entirety, including all charts and drawings.

In various embodiments, the present invention provides methods of designing ligands that bind to a plurality of members of a molecular family, where the ligands contain a common molecular scaffold. Thus, a compound set can be assayed for binding to a plurality of members of a molecular family, e.g., a protein family. One or more compounds that bind to a plurality of family members can be identified as molecular scaffolds. When the orientation of the scaffold at the binding site of the target molecules has been determined and chemically tractable structures have been identified, a set of ligands can be synthesized starting with one or a few molecular scaffolds to arrive at a plurality of ligands, wherein each ligand binds to a separate target molecule of the molecular family with altered or changed binding affinity or binding specificity relative to the scaffold. Thus, a plurality of drug lead molecules can be designed to individually target members of a molecular family based on the same molecular scaffold, and act on them in a specific manner.

D. Binding Assays

1. Use of Binding Assays

The methods of the present invention can involve assays that are able to detect the binding of compounds to a target molecule at a signal of at least about three times the standard deviation of the background signal, or at least about four times the standard deviation of the background signal. The assays can also include assaying compounds for low affinity binding to the target molecule. A large variety of assays indicative of binding are known for different target types and can be used for this invention. Compounds that act broadly across protein families are not likely to have a high affinity against individual targets, due to the broad nature of their binding. Thus, assays (e.g., as described herein) highly preferably allow for the identification of compounds that bind with low affinity, very low affinity, and extremely low affinity. Therefore, potency (or binding affinity) is not the primary, nor even the most important, indicia of identification of a potentially useful binding compound. Rather, even those compounds that bind with low affinity, very low affinity, or extremely low affinity can be considered as molecular scaffolds that can continue to the next phase of the ligand design process.

As indicated above, to design or discover scaffolds that act broadly across protein families, proteins of interest can be assayed against a compound collection or set. The assays can preferably be enzymatic or binding assays. In some embodiments it may be desirable to enhance the solubility of the compounds being screened and then analyze all compounds that show activity in the assay, including those that bind with low affinity or produce a signal with greater than about three times the standard deviation of the background signal. These assays can be any suitable assay such as, for example, binding assays that measure the binding affinity between two binding partners. Various types of screening assays that can be useful in the practice of the present invention are known in the art, such as those described in U.S. Pat. Nos. 5,763,198, 5,747,276, 5,877,007, 6,243,980, 6,294,330, and 6,294,330, each of which is hereby incorporated by reference in its entirety, including all charts and drawings.

In various embodiments of the assays at least one compound, at least about 5%, at least about 10%, at least about 15%, at least about 20%, or at least about 25% of the compounds can bind with low affinity. In many cases, up to about 20% of the compounds can show activity in the screening assay and these compounds can then be analyzed directly with high-throughput co-crystallography, computational analysis to group the compounds into classes with common structural properties (e.g., structural core and/or shape and polarity characteristics), and the identification of common chemical structures between compounds that show activity.

The person of ordinary skill in the art will realize that decisions can be based on criteria that are appropriate for the needs of the particular situation, and that the decisions can be made by computer software programs. Classes can be created containing almost any number of scaffolds, and the criteria selected can be based on increasingly exacting criteria until an arbitrary number of scaffolds is arrived at for each class that is deemed to be advantageous.

2. Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference: Vely F. et al., Methods in Molecular Biology., 2000, 121:313-21; Liparoto et al., J. Molecular Recognition., 1999, 12:316-21; Lipschultz et al., Methods. 2000, 20:310-8; Malmqvist., Biochemical Society Transactions, 1999, 27:335-40; Alfthan, 1998, Biosensors & Bioelectronics. 13:653-63; Fivash et al., Current Opinion in Biotechnology, 1998, 9:97-101; Price et al., 1998, Tumour Biology 19 Suppl 1:1-20; Malmqvist et al., Current Opinion in Chemical Biology., 1997, 1:378-83; O'Shannessy et al., Analytical Biochemistry. 1996, 236:275-83; Malmborg et al., 1995, J. Immunological Methods. 183:7-13; Van Regenmortel, Developments in Biological Standardization., 1994, 83:143-51; and O'Shannessy, Current Opinions in Biotechnology., 1994, 5:65-71.

BIAcore® uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

E. High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known enzyme modulator. Similarly, when ligands to a target are sought, known ligands of the target can be present in control/calibration assay wells.

F. Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of colorimetric assays for the detection of peroxides, as described in Gordon, A. J. and Ford, R. A., The Chemist's Companion: A Handbook Of Practical Data, Techniques, And References, John Wiley and Sons, N.Y., 1972, Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., Spectrophotometry and Spectrofluorometry: A Practical Approach, pp. 91-114, IRL Press Ltd. (1987); and Bell, Spectroscopy In Biochemistry, Vol. 1, pp. 155-194, CRC Press (1981).

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, Oreg.). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex® Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owickiet al., Application ofFluorescence Polarization Assays in High-Throughput Screening, in Genetic Engineering News, 1997, 17:27.

FP is particularly desirable since its readout is independent of the emission intensity (Checovich, W. J., et al., Nature 1995, 375:254-256; Dandliker, W. B., et al., Methods in Enzymology 1981, 74:3-28) and is thus insensitive to the presence of colored compounds that quench fluorescence emission. FP and FRET (see below) are well-suited for identifying compounds that block interactions between sphingolipid receptors and their ligands. See, for example, Parker et al., Development of high throughput screening assays using fluorescence polarization: nuclear receptor-ligand-binding and kinase/phosphatase assays, J. Biomol Screen, 2000, 5:77-88.

Fluorophores derived from sphingolipids that may be used in FP assays are commercially available. For example, Molecular Probes (Eugene, Oreg.) currently sells sphingomyelin and one ceramide flurophores. These are, respectively, N-(4,4-difluoro-5,7-dimethyl-4-bora-3a,4a-diaza-s-indacene-3-pentanoyl)sphingosyl phosphocholine (BODIPY® FL C5-sphingomyelin); N-(4,4-difluoro-5,7-dimethyl-4-bora-3a,4a-diaza-s-indacene-3-dodecanoyl)sphingosyl phosphocholine (BODIPY® FL C12-sphingomyelin); and N-(4,4-difluoro-5,7-dimethyl-4-bora-3 a,4a-diaza-s-indacene-3 -pentanoyl)sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION® fluorescence polarization system (Tecan AG, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., Curr. Biol. 1996, 6:178-182; Mitra et al., Gene, 1996, 173:13-17; and Selvin et al., Meth. Enzymol., 1995, 246:300-345. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., J. Lipid Res., 1997, 38:2365-2373; Kahl et al., Anal. Biochem., 1996, 243:282-283; Undenfriend et al., Anal. Biochem., 1987, 161:494-500). See also U.S. Pat. Nos. 4,626,513 and 4,568,649, and European Patent No. 0,154,734. One commercially available system uses FLASHPLATE® scintillant-coated plates (NEN Life Science Products, Boston, Mass.).

The target molecule can be bound to the scintillator plates by a variety of well known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT® microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., Anal. Biochem., 1998, 257:112-119).

Additionally, the assay can utilize AlphaScreen (amplified luminescent proximity homogeneous assay) format, e.g., AlphaScreening system (Packard BioScience). AlphaScreen is generally described in Seethala and Prabhavathi, Homogenous Assays: AlphaScreen, Handbook of Drug Screening, Marcel Dekkar Pub., 2001, pp. 106-110.

G. Assay Compounds and Molecular Scaffolds

As described above, preferred characteristics of a scaffold include being of low molecular weight (e.g., less than 350 daltons, or from about 100 to about 350 daltons, or from about 150 to about 300 daltons). Preferably clogP of a scaffold is from -1 to 8, more preferably less than 6, 5, or 4, most preferably less than 3. In particular embodiments the clogP is in a range −1 to an upper limit of 2, 3, 4, 5, 6, or 8; or is in a range of 0 to an upper limit of 2, 3, 4, 5, 6, or 8. Preferably the number of rotatable bonds is less than 5, more preferably less than 4. Preferably the number of hydrogen bond donors and acceptors is below 6, more preferably below 5. An additional criterion that can be useful is a Polar Surface Area of less than 100. Guidance that can be useful in identifying criteria for a particular application can be found in Lipinski et al., Advanced Drug Delivery Reviews, 1997, 23:3-25, which is hereby incorporated by reference in its entirety.

A scaffold will preferably bind to a given protein binding site in a configuration that causes substituent moieties of the scaffold to be situated in pockets of the protein binding site. Also, possessing chemically tractable groups that can be chemically modified, particularly through synthetic reactions, to easily create a combinatorial library can be a preferred characteristic of the scaffold. Also preferred can be having positions on the scaffold to which other moieties can be attached, which do not interfere with binding of the scaffold to the protein(s) of interest but do cause the scaffold to achieve a desirable property, for example, active transport of the scaffold to cells and/or organs, enabling the scaffold to be attached to a chromatographic column to facilitate analysis, or another desirable property. A molecular scaffold can bind to a target molecule with any affinity, such as binding with an affinity measurable as about three times the standard deviation of the background signal, or at high affinity, moderate affinity, low affinity, very low affinity, or extremely low affinity.

Thus, the above criteria can be utilized to select many compounds for testing that have the desired attributes. Many compounds having the criteria described are available in the commercial market, and may be selected for assaying depending on the specific needs to which the methods are to be applied. In some cases sufficiently large numbers of compounds may meet specific criteria that additional methods to group similar compounds may be helpful. A variety of methods to assess molecular similarity, such as the Tanimoto coefficient have been used, see Willett et al., J. Chemical Information and Computer Science, 1998, 38:983-996. These can be used to select a smaller subset of a group of highly structurally redundant compounds. In addition, cluster analysis based on relationships between the compounds, or structural components of the compound, can also be carried out to the same end; see Lance & Williams, Computer J., 1967, 9:373-380, Jarvis & Patrick IEEE Transactions in Computers, 1973, C-22:1025-1034 for clustering algorithms, and Downs et al. J. Chemical Information and Computer Sciences, 1994, 34:1094-1102 for a review of these methods applied to chemical problems. One method of deriving the chemical components of a large group of potential scaffolds is to virtually break up the compound at rotatable bonds so as to yield components of no less than 10 atoms. The resulting components may be clustered based on some measure of similarity, e.g. the Tanimoto coefficient, to yield the common component groups in the original collection of compounds. For each component group, all compounds containing that component may be clustered, and the resulting clusters used to select a diverse set of compounds containing a common chemical core structure. In this fashion, a useful library of scaffolds may be derived even from millions of commercial compounds.

A “compound library” or “library” is a collection of different compounds having different chemical structures. A compound library is screenable, that is, the compound library members therein may be subject to screening assays. In preferred embodiments, the library members can have a molecular weight of from about 100 to about 350 daltons, or from about 150 to about 350 daltons.

Libraries can contain at least one compound that binds to the target molecule at low affinity. Libraries of candidate compounds can be assayed by many different assays, such as those described above, e.g., a fluorescence polarization assay. Libraries may consist of chemically synthesized peptides, peptidomimetics, or arrays of combinatorial chemicals that are large or small, focused or nonfocused. By “focused” it is meant that the collection of compounds is prepared using the structure of previously characterized compounds and/or pharmacophores.

Compound libraries may contain molecules isolated from natural sources, artificially synthesized molecules, or molecules synthesized, isolated, or otherwise prepared in such a manner so as to have one or more moieties variable, e.g., moieties that are independently isolated or randomly synthesized. Types of molecules in compound libraries include but are not limited to organic compounds, polypeptides and nucleic acids as those terms are used herein, and derivatives, conjugates and mixtures thereof.

Compound libraries useful for the invention may be purchased on the commercial market or prepared or obtained by any means including, but not limited to, combinatorial chemistry techniques, fermentation methods, plant and cellular extraction procedures and the like (see, e.g., Cwirla et al., Biochemistry, 1990, 87:6378-6382; Houghten et al., Nature, 1991, 354:84-86; Lam et al., Nature, 1991, 354:82-84; Brenner et al., Proc. Natl. Acad. Sci. USA, 1992, 89:5381-5383; R. A. Houghten, Trends Genet., 1993, 9:235-239; E. R. Felder, Chimia, 1994, 48:512-541; Gallop et al., J. Med. Chem., 1994, 37:1233-1251; Gordon et al., J. Med. Chem., 1994, 37:1385-1401; Carell et al., Chem. Biol., 1995,3:171-183; Madden et al., Perspectives in Drug Discovery and Design 2:269-282; Lebl et al., Biopolymers, 1995, 37:177-198); small molecules assembled around a shared molecular structure; collections of chemicals that have been assembled by various commercial and noncommercial groups, natural products; extracts of marine organisms, fungi, bacteria, and plants.

Preferred libraries can be prepared in a homogenous reaction mixture, and separation of unreacted reagents from members of the library is not required prior to screening. Although many combinatorial chemistry approaches are based on solid state chemistry, liquid phase combinatorial chemistry is capable of generating libraries (Sun C M., Recent advances in liquid-phase combinatorial chemistry, Combinatorial Chemistry & High Throughput Screening, 1999, 2:299-318).

Libraries of a variety of types of molecules can be prepared in order to obtain members therefrom having one or more preselected attributes that can be prepared by a variety of techniques, including but not limited to parallel array synthesis (Houghton, Ann. Rev. Pharmacol. Toxicol., 2000, 40:273-82); solution-phase combinatorial chemistry (Merritt, Comb Chem High Throughput Screen, 1998, 1:57-72; Coe et al., Mol. Divers, 1998-99, 4:31-38; Sun, Comb Chem High Throughput Screenm, 1999, 2:299-318); synthesis on soluble polymer (Gravert et al., Curr Opin Chem Biol., 1997, 1:107-13); and the like. See, e.g., Dolle etal., J. Comb Chem., 1999, 1:235-82; and Kundu et al., Prog Drug Res., 1999, 53:89-156, Combinatorial chemistry: polymer supported synthesis of peptide and non-peptide libraries). Compounds may be clinically tagged for ease of identification (Chabala, Curr Opin Biotechnol., 1995 6:633-9, Solid-phase combinatorial chemistry and novel tagging methods for identifying leads).

The combinatorial synthesis of carbohydrates and libraries containing oligosaccharides has been described (Schweizer et al., Curr. Opin. Chem. Biol., 1999, 3:291-8, Combinatorial synthesis of carbohydrates). The synthesis of natural-product based compound libraries has been described (Wessjohann, Curr. Opin. Chem. Biol., 2000, 4:303-9).

Libraries of nucleic acids are prepared by various techniques, including by way of non-limiting example the ones described herein, for the isolation of aptamers. Libraries that include oligonucleotides and polyaminooligonucleotides (Markiewicz et al., Farmaco., 2000, 55:174-7) displayed on streptavidin magnetic beads are known. Nucleic acid libraries are known that can be coupled to parallel sampling and be deconvoluted without complex procedures such as automated mass spectrometry (Enjalbal et al., Mass Spectrometry Reviews., 2000, 19:139-61) and parallel tagging. (Perrin D M., Combinatorial Chemistry & High Throughput Screening, 3:243-69).

Peptidomimetics are identified using combinatorial chemistry and solid phase synthesis (Kim H O. Kahn M., Combinatorial Chemistry & High Throughput Screening, 2000, 3:167-83; al-Obeidi, Mol Biotechnol., 1998, 9:205-23). The synthesis may be entirely random or based in part on a known polypeptide.

Polypeptide libraries can be prepared according to various techniques. In brief, phage display techniques can be used to produce polypeptide ligands (Gram H., Combinatorial Chemistry & High Throughput Screening, 1999, 2:19-28) that may be used as the basis for synthesis of peptidomimetics. Polypeptides, constrained peptides, proteins, protein domains, antibodies, single chain antibody fragments, antibody fragments, and antibody combining regions are displayed on filamentous phage for selection.

Large libraries of individual variants of human single chain Fv antibodies have been produced. See, e.g., Siegel et al., J. Molecular Biology 2000, 302:285-93; Poul et al., J. Molecular Biology. 2000, 301:1149-61; Amersdorfer et al., Methods in Molecular Biology. 2001, 145:219-40; Hughes-Jones et al., British J. Haematology., 1999, 105:811-6; McCall et al., Immunotechnology. 1998, 4:71-87; Sheets et al., (published erratum appears in Proc Natl Acad Sci USA 1999 96:795), 1998, Proc Natl Acad Sci USA 95:6157-62).

Focused or smart chemical and pharmacophore libraries can be designed with the help of sophisticated strategies involving computational chemistry (e.g., Kundu et al., Progress in Drug Research 1999, 53:89-156) and the use of structure-based ligands using database searching and docking, de novo drug design and estimation of ligand binding affinities (Joseph-McCarthy D., Pharmacology & Therapeutics 1999, 84:179-91; Kirkpatrick et al., Combinatorial Chemistry & High Throughput Screening., 1999, 2:211-21; Eliseev & Lehn, Current Topics in Microbiology & Immunology, 1999, 243:159-72; Bolger et al., Methods Enz. 1991, 203:21-45; Martin, Methods Enz. 1991, 203:587-613; Neidle et al., Methods Enz. 1991, 203:433-458; U.S. Pat. No. 6,178,384).

Selecting a library of potential scaffolds and a set of assays measuring binding to representative target molecules which are in a particular protein family thus allows the creation of a data set profiling binding of the library to the target protein family. Groups of scaffolds with different sets of binding properties can be identified using the information within this dataset. Thus, groups of scaffolds binding to one, two or three members of the family may be selected for particular applications.

In many cases, a group of scaffolds exhibiting binding to two or more members of a target protein family will contain scaffolds with a greater likelihood that such binding results from specific interactions with the individual target proteins. This would be expected to substantially reduce the effect of so-called “promiscuous inhibitors” which severely complicate the interpretation of screening assays (see McGovern et al., J. Med. Chem. 2002, 45:1712-22). Thus, in many preferred applications the property of displaying binding to multiple target molecules in a protein family may be used as a selection criteria to identify molecules with desirable properties. In addition, groups of scaffolds binding to specific subsets of a set of potential target molecules may be selected. Such a case would include the subset of scaffolds that bind to any two of three or three of five members of a target protein family.

Such subsets may also be used in combination or opposition to further define a group of scaffolds that have additional desirable properties. This would be of significant utility in cases where inhibiting some members of a protein family had known desirable effects, such as inhibiting tumor growth, whereas inhibiting other members of the protein family which were found to be essential for normal cell function would have undesirable effects. A criteria that would be useful in such a case includes selecting the subset of scaffolds binding to any two of three desirable target molecules and eliminating from this group any that bound to more than one of any three undesirable target molecules.

Representative molecular scaffolds of the invention include, but are not limited to compounds of Formula I:
A-L¹-B-L²-C Formula I
wherein:
A is optional, and if present is selected from the group consisting of aryl, heteroaryl, and derivatives thereof optionally substituted with one, two, or three substituents as defined in [0287] and [02881 attached at any available atom to produce a stable compound;
L¹ is optional, and if present is a divalent C₁₋₃ alkylene radical;
B is selected from the group consisting of aryl, heteroaryl, and derivatives thereof optionally substituted with one, two, or three substituents as defined in [0287] and [0288] attached at any available atom to produce a stable compound;
L² is optional, and if present is selected from the group consisting of divalent C₁₋₃ alkylene radical and —C₁₋₃ alkylene-O-; and
C is optional, and if present is selected from the group consisting of aryl, heteroaryl, and derivatives thereof optionally substituted with one, two, or three substituents as defined in [0287] and [0288] attached at any available atom to produce a stable compound.

The following compound obtained from Chembridge (San Diego, Calif.), 5-(4-chloro-3-methyl-phenoxymethyl)-3-thiophen-2-ylmethyl-[1,2,4]oxadiazole, is an example of a possible molecular scaffold compound for development of ligands that bind to SF-1 or LRH-1:

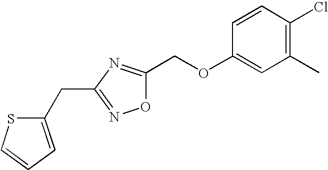

H. Crystallography

After binding compounds have been determined, the orientation of compound bound to target is determined. Preferably this determination involves crystallography on co-crystals of molecular scaffold compounds with target. Most protein crystallographic platforms can preferably be designed to analyze up to about 500 co-complexes of compounds, ligands, or molecular scaffolds bound to protein targets due to the physical parameters of the instruments and convenience of operation.

If the number of scaffolds that have binding activity exceeds a number convenient for the application of crystallography methods, the scaffolds can be placed into groups based on having at least one common chemical structure or other desirable characteristics, and representative compounds can be selected from one or more of the classes. Classes can be made with increasingly exacting criteria until a desired number of classes (e.g., 10, 20, 50, 100, 200, 300, 400, 500) is obtained. The classes can be based on chemical structure similarities between molecular scaffolds in the class, e.g., all possess a pyrrole ring, benzene ring, or other chemical feature. Likewise, classes can be based on shape characteristics, e.g., space-filling characteristics.

The co-crystallography analysis can be performed by co-complexing each scaffold with its target, e.g., at concentrations of the scaffold that showed activity in the screening assay. This co-complexing can, for example, be accomplished with the use of low percentage organic solvents with the target molecule and then concentrating the target with each of the scaffolds. In preferred embodiments these solvents are less than 5% organic solvent such as dimethyl sulfoxide (DMSO), ethanol, methanol, or ethylene glycol in water or another aqueous solvent.

Each scaffold complexed to the target molecule can then be screened with a suitable number of crystallization screening conditions at appropriate temperature, e.g., both 4 and 20 degrees. In preferred embodiments, about 96 crystallization screening conditions can be performed in order to obtain sufficient information about the co-complexation and crystallization conditions, and the orientation of the scaffold at the binding site of the target molecule. Crystal structures can then be analyzed to determine how the bound scaffold is oriented physically within the binding site or within one or more binding pockets of the molecular family member.

It is desirable to determine the atomic coordinates of the compounds bound to the target proteins in order to determine which is a most suitable scaffold for the protein family. X-ray crystallographic analysis is therefore most preferable for determining the atomic coordinates. Those compounds selected can be further tested with the application of medicinal chemistry. Compounds can be selected for medicinal chemistry testing based on their binding position in the target molecule. For example, when the compound binds at a binding site, the compound's binding position in the binding site of the target molecule can be considered with respect to the chemistry that can be performed on chemically tractable structures or sub-structures of the compound, and how such modifications on the compound are expected to interact with structures or sub-structures on the binding site of the target. Thus, one can explore the binding site of the target and the chemistry of the scaffold in order to make decisions on how to modify the scaffold to arrive at a ligand with higher potency and/or selectivity.

The structure of the target molecule bound to the compound may also be superimposed or aligned with other structures of members of the same protein family. In this way modifications of the scaffold can be made to enhance the binding to members of the target family in general, thus enhancing the utility of the scaffold library. Different useful alignments may be generated, using a variety of criteria such as minimal RMSD superposition of alpha-carbons or backbone atoms of homologous or structurally related regions of the proteins.

These processes allow for more direct design of ligands, by utilizing structural and chemical information obtained directly from the co-complex, thereby enabling one to more efficiently and quickly design lead compounds that are likely to lead to beneficial drug products. In various embodiments it may be desirable to perform co-crystallography on all scaffolds that bind, or only those that bind with a particular affinity, for example, only those that bind with high affinity, moderate affinity, low affinity, very low affinity, or extremely low affinity. It may also be advantageous to perform co-crystallography on a selection of scaffolds that bind with any combination of affinities.

Standard X-ray protein diffraction studies such as by using a Rigaku RU-200® (Rigaku, Tokyo, Japan) with an X-ray imaging plate detector or a synchrotron beam-line can be performed on co-crystals and the diffraction data measured on a standard X-ray detector, such as a CCD detector or an X-ray imaging plate detector.

Performing X-ray crystallography on about 200 co-crystals should generally lead to about 50 co-crystal structures, which should provide about 10 scaffolds for validation in chemistry, which should finally result in about 5 selective leads for target molecules.

Additives that promote co-crystallization can of course be included in the target molecule formulation in order to enhance the formation of co-crystals. In the case of proteins or enzymes, the scaffold to be tested can be added to the protein formulation, which is preferably present at a concentration of approximately 1 mg/ml. The formulation can also contain between 0%-10% (v/v) organic solvent, e.g. DMSO, methanol, ethanol, propane diol, or 1,3 dimethyl propane diol (MPD) or some combination of those organic solvents. Compounds are preferably solubilized in the organic solvent at a concentration of about 100 mM and added to the protein sample at a concentration of about 1-10 mM. The protein-compound complex is then concentrated to a final concentration of protein of from about 5 to about 20 mg/ml. The complexation and concentration steps can conveniently be performed using a 96 well formatted concentration apparatus (e.g., Amicon Inc., Piscataway, N.J.). Buffers and other reagents present in the formulation being crystallized can contain other components that promote crystallization or are compatible with crystallization conditions, such as DTT, propane diol, glycerol.

The crystallization experiment can be set-up by placing small aliquots of the concentrated protein-compound complex (e.g., 1 μl) in a 96 well format and sampling under 96 crystallization conditions. (Other formats can also be used, for example, plates with fewer or more wells.) Crystals can typically be obtained using standard crystallization protocols that can involve the 96 well crystallization plate being placed at different temperatures. Co-crystallization varying factors other than temperature can also be considered for each protein-compound complex if desirable. For example, atmospheric pressure, the presence or absence of light or oxygen, a change in gravity, and many other variables can all be tested. The person of ordinary skill in the art will realize other variables that can advantageously be varied and considered. Conveniently, commercially available crystal screening plates with specified conditions in individual wells can be utilized.

I. Virtual Assays

As described above, virtual assays or compound design techniques are useful for identification and design of modulators; such techniques are also applicable to a molecular scaffold method. Commercially available software that generates three-dimensional graphical representations of the complexed target and compound from a set of coordinates provided can be used to illustrate and study how a compound is oriented when bound to a target. (e.g., InsightII®, Accelrys, San Diego, Calif.; or Sybyl®, Tripos Associates, St. Louis, Mo.). Thus, the existence of binding pockets at the binding site of the targets can be particularly useful in the present invention. These binding pockets are revealed by the crystallographic structure determination and show the precise chemical interactions involved in binding the compound to the binding site of the target. The person of ordinary skill will realize that the illustrations can also be used to decide where chemical groups might be added, substituted, modified, or deleted from the scaffold to enhance binding or another desirable effect, by considering where unoccupied space is located in the complex and which chemical substructures might have suitable size and/or charge characteristics to fill it. The person of ordinary skill will also realize that regions within the binding site can be flexible and its properties can change as a result of scaffold binding, and that chemical groups can be specifically targeted to those regions to achieve a desired effect. Specific locations on the molecular scaffold can be considered with reference to where a suitable chemical substructure can be attached and in which conformation, and which site has the most advantageous chemistry available.

An understanding of the forces that bind the compounds to the target proteins reveals which compounds can most advantageously be used as scaffolds, and which properties can most effectively be manipulated in the design of ligands. The person of ordinary skill will realize that steric, ionic, polar, hydrogen bond, and other forces can be considered for their contribution to the maintenance or enhancement of the target-compound complex. Additional data can be obtained with automated computational methods, such as docking and/or molecular dynamics simulations, which can afford a measure of the energy of binding. In addition, to account for other effects such as entropies of binding and desolvation penalties, methods which provide a measure of these effects can be integrated into the automated computational approach. The compounds selected can be used to generate information about the chemical interactions with the target or for elucidating chemical modifications that can enhance selectivity of binding of the compound.

An exemplary calculation of binding energies between protein-ligand complexes can be obtained using the FlexX score (an implementation of the Bohm scoring function) within the Tripos software suite (Tripos Associates, St. Louis, Mo.). The form for that equation is shown below:
ΔGbind=ΔGtr+ΔGhb+ΔGion+ΔGlipo+ΔGarom+ΔGrot
where: ΔGtr is a constant term that accounts for the overall loss of rotational and translational entropy of the ligand, ΔGhb accounts for hydrogen bonds formed between the ligand and protein, ΔGion accounts for the ionic interactions between the ligand and protein, ΔGlipo accounts for the lipophilic interaction that corresponds to the protein-ligand contact surface, ΔGarom accounts for interactions between aromatic rings in the protein and ligand, and ΔGrot accounts for the entropic penalty of restricting rotatable bonds in the ligand upon binding. The calculated binding energy for compounds that bind strongly to a given target will likely be lower than −25 kcal/mol, while the calculated binding affinity for a good scaffold or an unoptimized compound will generally be in the range of −15 to −20. The penalty for restricting a linker such as the ethylene glycol or hexatriene is estimated as typically being in the range of +5 to +15.

This method estimates the free energy of binding that a lead compound should have to a target protein for which there is a crystal structure, and it accounts for the entropic penalty of flexible linkers. It can therefore be used to estimate the penalty incurred by attaching linkers to molecules being screened and the binding energy that a lead compound must attain in order to overcome the penalty of the linker. The method does not account for solvation, and the entropic penalty is likely overestimated when the linkers are bound to the solid phase through an additional binding complex, e.g., a biotin:streptavidin complex.

Another exemplary method for calculating binding energies is the MM-PBSA technique (Massova & Kollman, J. Amer. Chem. Soc., 1999, 121:8133-43; Chong et al., Proc. of the Natl. Acad. of Sci. USA, 1999, 96:14330-5; Donini & Kollman, J. Med. Chem. 2000, 43:4180-8). This method uses a Molecular Dynamics approach to generate many sample configurations of the compound and complexed target molecule, then calculates an interaction energy using the well-known AMBER force field (Cornell, et al., J. Amer. Chem. Soc., 1995, 117:5179-97) with corrections for desolvation and entropy of binding from the ensemble.

Use of this method yields binding energies highly correlated with those found experimentally. The absolute binding energies calculated with this method are reasonably accurate, and the variation of binding energies is approximately linear with a slope of 1±0.5. Thus, the binding energies of compounds interacting strongly with a given target will be lower than about −8 kcal/mol, while a binding energy of a good scaffold or unoptimized compound will be in the range of −3 to −7 kcal/mol.

Computer models, such as homology models (i.e., based on a known, experimentally derived structure) can be constructed using data from the co-crystal structures. A computer program such as Modeller (Accelrys, San Diego Calif.) may be used to assign the three dimensional coordinates to a protein sequence using an alignment of sequences and a set or sets of template coordinates. When the target molecule is a protein or enzyme, preferred co-crystal structures for making homology models contain high sequence identity in the binding site of the protein sequence being modeled, and the proteins will preferentially also be within the same class and/or fold family. Knowledge of conserved residues in active sites of a protein class can be used to select homology models that accurately represent the binding site. Homology models can also be used to map structural information from a surrogate protein where an apo or co-crystal structure exists to the target protein.

Virtual screening methods, such as docking, can also be used to predict the binding configuration and affinity of scaffolds, compounds, and/or combinatorial library members to homology models. Using this data, and carrying out “virtual experiments” using computer software can save substantial resources and allow the person of ordinary skill to make decisions about which compounds can be suitable scaffolds or ligands, without having to actually synthesize the ligand and perform co-crystallization. Decisions thus can be made about which compounds merit actual synthesis and co-crystallization. An understanding of such chemical interactions aids in the discovery and design of drugs that interact more advantageously with target proteins and/or are more selective for one protein family member over others. Thus, applying these principles, compounds with superior properties can be discovered.

Another commonly-used virtual screening method is pharmacophore-based search. Crystal structures of a target protein allow the identification of pharmacophore features in the three-dimensional space using programs such as Catalyst (Accelrys, San Diego Calif.) or MOE (CCG, Montreal, Canada). Programs such as Catalyst and MOE can be used to search a large collection of existing compounds or virtual compounds that satisfy all or a subset of the defined pharmacophore features. Use of these data allows the person of ordinary skill to make decisions about which compounds may have activity for the target. These compounds and the binding hypothesis generated by using pharmacophore-based methods can then be used as a starting point to design compounds with better properties.

J. Ligand Design and Preparation

The design and preparation of ligands can be performed with or without structural and/or co-crystallization data by considering the chemical structures in common between the active scaffolds of a set. In this process structure-activity hypotheses can be formed and those chemical structures found to be present in a substantial number of the scaffolds, including those that bind with low affinity, can be presumed to have some effect on the binding of the scaffold. This binding can be presumed to induce a desired biochemical effect when it occurs in a biological system (e.g., a treated mammal). New or modified scaffolds or combinatorial libraries derived from scaffolds can be tested to disprove the maximum number of binding and/or structure-activity hypotheses. The remaining hypotheses can then be used to design ligands that achieve a desired binding and biochemical effect.

But in many cases it will be preferred to have co-crystallography data for consideration of how to modify the scaffold to achieve the desired binding effect (e.g., binding at higher affinity or with higher selectivity). Using the case of proteins and enzymes, co-crystallography data shows the binding pocket of the protein with the molecular scaffold bound to the binding site, and it will be apparent that a modification can be made to a chemically tractable group on the scaffold. For example, a small volume of space at a protein binding site or pocket might be filled by modifying the scaffold to include a small chemical group that fills the volume. Filling the void volume can be expected to result in a greater binding affinity, or the loss of undesirable binding to another member of the protein family. Similarly, the co-crystallography data may show that deletion of a chemical group on the scaffold may decrease a hindrance to binding and result in greater binding affinity or specificity.

Various software packages have implemented techniques which facilitate the identification and characterization of interactions of potential binding sites from complex structure, or from an apo structure of a target molecule, i.e. one without a compound bound (e.g. SiteID, Tripos Associates, St. Louis Mo. and SiteFinder, Chemical Computing Group, Montreal Canada, GRID, Molecular Discovery Ltd., London UK). Such techniques can be used with the coordinates of a complex between the scaffold of interest and a target molecule, or these data in conjunction with data for a suitably aligned or superimposed related target molecule, in order to evaluate changes to the scaffold that would enhance binding to the desired target molecule structure or structures. Molecular Interaction Field-computing techniques, such as those implemented in the program GRID, result in energy data for particular positive and negative binding interactions of different computational chemical probes being mapped to the vertices of a matrix in the coordinate space of the target molecule. These data can then be analyzed for areas of substitution around the scaffold binding site which are predicted to have a favorable interaction for a particular target molecule. Compatible chemical substitution on the scaffold e.g. a methyl, ethyl or phenyl group in a favorable interaction region computed from a hydrophobic probe, would be expected to result in an improvement in affinity of the scaffold. Conversely, a scaffold could be made more selective for a particular target molecule by making such a substitution in a region predicted to have an unfavorable hydrophobic interaction in a second, related undesirable target molecule.

It can be desirable to take advantage of the presence of a charged chemical group located at the binding site or pocket of the protein. For example, a positively charged group can be complemented with a negatively charged group introduced on the molecular scaffold. This can be expected to increase binding affinity or binding specificity, thereby resulting in a more desirable ligand. In many cases, regions of protein binding sites or pockets are known to vary from one family member to another based on the amino acid differences in those regions. Chemical additions in such regions can result in the creation or elimination of certain interactions (e.g., hydrophobic, electrostatic, or entropic) that allow a compound to be more specific for one protein target over another or to bind with greater affinity, thereby enabling one to synthesize a compound with greater selectivity or affinity for a particular family member. Additionally, certain regions can contain amino acids that are known to be more flexible than others. This often occurs in amino acids contained in loops connecting elements of the secondary structure of the protein, such as alpha helices or beta strands. Additions of chemical moieties can also be directed to these flexible regions in order to increase the likelihood of a specific interaction occurring between the protein target of interest and the compound. Virtual screening methods can also be conducted in silico to assess the effect of chemical additions, subtractions, modifications, and/or substitutions on compounds with respect to members of a protein family or class.

The addition, subtraction, or modification of a chemical structure or sub-structure to a scaffold can be performed with any suitable chemical moiety. For example the following moieties, which are provided by way of example and are not intended to be limiting, can be utilized: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (e.g., forming a ketone or N-oxide) or a sulphur atom (e.g., forming a thiol, thione, di-alkylsulfoxide or sulfone) are all examples of moieties that can be utilized.

Additional examples of structures or sub-structures that may be utilized are an aryl optionally substituted with one, two, or three substituents independently selected from the group consisting of alkyl, alkoxy, halogen, trihalomethyl, carboxylate, nitro, and ester moieties; an amine of formula —NX₂X₃, where X₂ and X₃ are independently selected from the group consisting of hydrogen, saturated or unsaturated alkyl, and homocyclic or heterocyclic ring moieties; halogen or trihalomethyl; a ketone of formula —COX₄, where X₄ is selected from the group consisting of alkyl and homocyclic or heterocyclic ring moieties; a carboxylic acid of formula —(X₅)ₙCOOH or ester of formula (X₆)ₙCOOX₇, where X₅, X₆, and X₇ and are independently selected from the group consisting of alkyl and homocyclic or heterocyclic ring moieties and where n is 0 or 1; an alcohol of formula (X₈)ₙOH or an alkoxy moiety of formula —(X₈)ₙOX₉, where X₈ and X₉ are independently selected from the group consisting of saturated or unsaturated alkyl and homocyclic or heterocyclic ring moieties, wherein said ring is optionally substituted with one or more substituents independently selected from the group consisting of alkyl, alkoxy, halogen, trihalomethyl, carboxylate, nitro, and ester and where n is 0 or 1; an amide of formula NHCOX₁₀, where X₁₀ is selected from the group consisting of alkyl, hydroxyl, and homocyclic or heterocyclic ring moieties, wherein said ring is optionally substituted with one or more substituents independently selected from the group consisting of alkyl, alkoxy, halogen, trihalomethyl, carboxylate, nitro, and ester; SO₂, NX₁₁, X₁₂, where X₁₁ and X₁₂ are selected from the group consisting of hydrogen, alkyl, and homocyclic or heterocyclic ring moieties; a homocyclic or heterocyclic ring moiety optionally substituted with one, two, or three substituents independently selected from the group consisting of alkyl, alkoxy, halogen, trihalomethyl, carboxylate, nitro, and ester moieties; an aldehyde of formula —COH; a sulfone of formula —SO₂X₁₃, where X₁₃ is selected from the group consisting of saturated or unsaturated alkyl and homocyclic or heterocyclic ring moieties; and a nitro of formula —NO₂.

K. Identification of Binding Characteristics of Binding Compounds

It can also be beneficial in selecting compounds for testing to first identify binding characteristics that a ligand should advantageously possess. This can be accomplished by analyzing the interactions that a plurality of different binding compounds have with a particular target, e.g., interactions with one or more conserved residues in the binding site. These interactions are identified by considering the nature of the interacting moieties. In this way, atoms or groups that can participate in hydrogen bonding, polar interactions, charge-charge interactions, and the like are identified based on known structural and electronic factors.

L. Identification of Energetically Allowed Sites for Attachment

In addition to the identification and development of ligands, determination of the orientation of a molecular scaffold or other binding compound in a binding site allows identification of energetically allowed sites for attachment of the binding molecule to another component. For such sites, any free energy change associated with the presence of the attached component should not destablize the binding of the compound to the target to an extent that will disrupt the binding. Preferably, the binding energy with the attachment should be at least 4 kcal/mol., more preferably at least 6, 8, 10, 12, 15, or 20 kcal/mol. Preferably, the presence of the attachment at the particular site reduces binding energy by no more than 3, 4, 5, 8, 10, 12, or 15 kcal/mol.

In many cases, suitable attachment sites will be those that are exposed to solvent when the binding compound is bound in the binding site. In some cases, attachment sites can be used that will result in small displacements of a portion of the enzyme without an excessive energetic cost. Exposed sites can be identified in various ways. For example, exposed sites can be identified using a graphic display or 3-dimensional model. In a graphic display, such as a computer display, an image of a compound bound in a binding site can be visually inspected to reveal atoms or groups on the compound that are exposed to solvent and oriented such that attachment at such atom or group would not preclude binding of the enzyme and binding compound. Energetic costs of attachment can be calculated based on changes or distortions that would be caused by the attachment as well as entropic changes.

Many different types of components can be attached. Persons with skill are familiar with the chemistries used for various attachments. Examples of components that can be attached include, without limitation: solid phase components such as beads, plates, chips, and wells; a direct or indirect label; a linker, which may be a traceless linker; among others. Such linkers can themselves be attached to other components, e.g., to solid phase media, labels, and/or binding moieties.

The binding energy of a compound and the effects on binding energy for attaching the molecule to another component can be calculated approximately by manual calculation, or by using any of a variety of available computational virtual assay techniques, such as docking or molecular dynamics simulations. A virtual library of compounds derived from the attachment of components to a particular scaffold can be assembled using a variety of software programs (such as Afferent, MDL Information Systems, San Leandro, Calif. or CombiLibMaker, Tripos Associates, St. Louis, Mo.). This virtual library can be assigned appropriate three dimensional coordinates using software programs (such as Concord, Tripos Associates, St. Louis, Mo. or Omega, Openeye Scientific Software, Santa Fe, N.Mex.). These structures may then be submitted to the appropriate computational technique for evaluation of binding energy to a particular target molecule. This information can be used for purposes of prioritizing compounds for synthesis, for selecting a subset of chemically tractable compounds for synthesis, and for providing data to correlate with the experimentally determined binding energies for the synthesized compounds.

The crystallographic determination of the orientation of the scaffold in the binding site specifically enables more productive methods of assessing the likelihood of the attachment of a particular component resulting in an improvement in binding energy. Such an example is shown for a docking-based strategy in Haque et al., (J. Med. Chem. 1999, 42:1428-40), wherein an “Anchor and Grow” technique which relied on a crystallographically determined fragment of a larger molecule, potent and selective inhibitors were rapidly created. The use of a crystallographically characterized small molecule fragment in guiding the selection of productive compounds for synthesis has also been demonstrated in Boehm et al., J. Med. Chem. 2000, 43:2664-74. An illustration of the use of crystallographic data and molecular dynamics simulations in the prospective assessment of inhibitor binding energies can be found in Pearlman and Charifson, J. Med. Chem. 2001, 44, 3417-23. Another important class of techniques which rely on a well defined structural starting point for computational design is the combinatorial growth algorithm based systems, such as the GrowMol program (Bohacek & McMartin, J. Amer. Chem. Soc., 1994, 116:5560-71. These techniques have been used to enable the rapid computational evolution of virtual inhibitor computed binding energies, and directly led to more potent synthesized compounds whose binding mode was validated crystallographically (see Organic Letters, 2001, 3:2309-2312).

1. Linkers

Linkers suitable for use in the invention can be of many different types. Linkers can be selected for particular applications based on factors such as linker chemistry compatible for attachment to a binding compound and to another component utilized in the particular application. Additional factors can include, without limitation, linker length, linker stability, and ability to remove the linker at an appropriate time. Exemplary linkers include, but are not limited to, hexenyl, hexatrienyl, ethylene glycol, and peptide linkers. Traceless linkers can also be used, e.g., as described in Plunkett & Ellman., J. Org. Chem., 1995, 60:6006.

Typical functional groups, that are utilized to link binding compound(s), include, but not limited to, carboxylic acid, amine, hydroxyl, and thiol. (Examples can be found in Solid-supported combinatorial and parallel synthesis of small molecular weight compound libraries; Tetrahedron Organic Chemistry Series 1998, Vol.17:85; Pergamon).

2. Labels

As indicated above, labels can also be attached to a binding compound or to a linker attached to a binding compound. Such attachment may be direct (attached directly to the binding compound) or indirect (attached to a component that is directly or indirectly attached to the binding compound). Such labels allow detection of the compound either directly or indirectly. Attachment of labels can be performed using conventional chemistries. Labels can include, for example, fluorescent labels, radiolabels, light scattering particles, light absorbent particles, magnetic particles, enzymes, and specific binding agents (e.g., biotin or an antibody target moiety).

3. Solid Phase Media

Additional examples of components that can be attached directly or indirectly to a binding compound include various solid phase media. Similar to attachment of linkers and labels, attachment to solid phase media can be performed using conventional chemistries. Such solid phase media can include, for example, small components such as beads, nanoparticles, and fibers (e.g., in suspension or in a gel or chromatographic matrix). Likewise, solid phase media can include larger objects such as plates, chips, slides, and tubes. In many cases, the binding compound will be attached in only a portion of such an objects, e.g., in a spot or other local element on a generally flat surface or in a well or portion of a well.

IV. Organic Synthetic Techniques

The versatility of computer-based modulator design and identification lies in the diversity of structures screened by the computer programs. The computer programs can search databases that contain very large numbers of molecules and can modify modulators already complexed with the enzyme with a wide variety of chemical functional groups. A consequence of this chemical diversity is that a potential modulator of a biomolecular function may take a chemical form that is not predictable. A wide array of organic synthetic techniques exist in the art to meet the challenge of constructing these potential modulators. Many of these organic synthetic methods are described in detail in standard reference sources utilized by those skilled in the art. One example of such a reference is March, 1994, Advanced Organic Chemistry; Reactions, Mechanisms and Structure, New York, McGraw Hill. Thus, the techniques useful to synthesize a potential modulator of biomolecular function identified by computer-based methods are readily available to those skilled in the art of organic chemical synthesis.

V. Isomers, Prodrugs, and Active Metabolites

The present invention concerns compounds that can be describes with generic formulas and specific compounds. In addition, such compounds may exist in a number of different forms or derivatives, all within the scope of the present invention. These include, for example, tautomers, stereoisomers, racemic mixtures, regioisomers, salts, prodrugs (e.g., carboxylic acid esters), solvated forms, different crystal forms or polymorphs, and active metabolites.

A. Tautomers, Stereoisomers, Regioisomers, and Solvated Forms

It is understood that certain compounds may exhibit tautomerism. In such cases, the formula drawings within this specification expressly depict only one of the possible tautomeric forms It is therefore to be understood that within the invention the formulas are intended to represent any tautomeric form of the depicted compounds and are not to be limited merely to the specific tautomeric form depicted by the formula drawings.

Likewise, some of the compounds according to the present invention may exist as stereoisomers, i.e. they have the same sequence of covalently bonded atoms and differ in the spatial orientation of the atoms. For example, the compounds may be optical stereoisomers, which contain one or more chiral centers, and therefore, may exist in two or more stereoisomeric forms (e.g. entantiomers or diastereomers). Thus, such compounds may be present as single stereoisomers (i.e., essentially free of other stereoisomers), racemates, and/or mixtures of enantiomers and/or diastereomers. As another example, stereoisomers include geometric isomers, such as cis- or trans-orientation of substituents on adjacent carbons of a double bond. All such single stereoisomers, racemates and mixtures thereof are intended to be within the scope of the present invention. Unless specified to the contrary, all such steroisomeric forms are included within the formulas provided herein.

In certain embodiments, a chiral compound of the present invention is in a form that contains at least 80% of a single isomer (60% enantiomeric excess (“e.e.”) or diastereomeric excess (“d.e.”)), or at least 85% (70% e.e. or d.e.), 90% (80% e.e. or d.e.), 95% (90% e.e. or d.e.), 97.5% (95% e.e. or d.e.), or 99% (98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In certain embodiments, the compound is present in optically pure form.

For compounds in which synthesis involves addition of a single group at a double bond, particularly a carbon-carbon double bond, the addition may occur at either of the double bond-linked atoms. For such compounds, the present invention includes both such regioisomers.

Additionally, the formulas are intended to cover solvated as well as unsolvated forms of the identified structures. For example, the indicated structures include both hydrated and non-hydrated forms. Other examples of solvates include the structures in combination with isopropanol, ethanol, methanol, DMSO, ethyl acetate, acetic acid, or ethanolamine.

B. Prodrugs and Metabolites

For compounds useful in the present invention, the invention also includes prodrugs (generally pharmaceutically acceptable prodrugs), active metabolic derivatives (active metabolites), and their pharmaceutically acceptable salts.

In this context, prodrugs are compounds or pharmaceutically acceptable salts thereof which, when metabolized under physiological conditions or when converted by solvolysis, yield the desired active compound. Typically, the prodrug is inactive, or less active than the active compound, but may provide advantageous handling, administration, or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. A common example is an alkyl ester of a carboxylic acid.

As described in The Practice ofMedicinal Chemistry, Ch. 31-32 (Ed. Wermuth, Academic Press, San Diego, Calif., 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types:

Oxidative reactions: Oxidative reactions are exemplified without limitation to reactions such as oxidation of alcohol, carbonyl, and acid functions, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

Reductive reactions: Reductive reactions are exemplified without limitation to reactions such as reduction of carbonyl groups, reduction of alcoholic groups and carbon-carbon double bonds, reduction of nitrogen-containing functions groups, and other reduction reactions.

Reactions without change in the state of oxidation: Reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Pat. Pub. No. 2004/0077595, U.S. Ser. No. 10/656,838, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, The Practice ofMedicinal Chemistry, Ch. 31-32, Ed. Wermuth, Academic Press, San Diego, Calif., 2001.

Prodrugs may proceed from prodrug form to active form in a single step or may have one or more intermediate forms which may themselves have activity or may be inactive.

Metabolites, e.g., active metabolites overlap with prodrugs as described above, e.g., bioprecursor prodrugs. Thus, such metabolites are pharmacologically active compounds or compounds that further metabolize to pharmacologically active compounds that are derivatives resulting from metabolic process in the body of a subject or patient. Of these, active metabolites are such pharmacologically active derivative compounds. For prodrugs, the prodrug compounds is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al., J. Med Chem., 1997, 40:2011-2016; Shan et al., J. Pharm Sci 86:756-757; Bagshawe, Drug Dev Res., 1995, 34:220-230; Wermuth, (supra).

C. Pharmaceutically Acceptable Salts

Compounds can be formulated as or be in the form of pharmaceutically acceptable salts. Pharmaceutically acceptable salts are non-toxic salts in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

Thus, for example, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

Unless specified to the contrary, specification of a compound herein includes pharmaceutically acceptable salts of such compound.

D. Polymorphic forms

In the case of agents that are solids, it is understood by those skilled in the art that the compounds and salts may exist in different crystal or polymorphic forms, all of which are intended to be within the scope of the present invention and specified formulas.

VI. Administration

The methods and compounds will typically be used in therapy for human patients. However, they may also be used to treat similar or identical diseases in other vertebrates, e.g., mammals such as other primates, sports animals, bovines, equines, porcines, ovines, and pets such as dogs and cats.

Suitable dosage forms, in part, depend upon the use or the route of administration, for example, oral, transdermal, transmucosal, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in Remington: The Science and Practice of Pharmacy, 21^{st} edition, Lippincott, Williams and Wilkins, Philadelphia, Pa., 2005 (hereby incorporated by reference herein).

Carriers or excipients can be used to produce pharmaceutical compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, or transdermal. Oral administration is preferred. For oral administration, for example, the compounds can be formulated into conventional oral dosage forms such as capsules, tablets, and liquid preparations such as syrups, elixirs, and concentrated drops.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/orsubcutaneous. For injection, the compounds of the invention are formulated in sterile liquid solutions, preferably in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The amounts of various compound to be administered can be determined by standard procedures taking into account factors such as the compound IC₅₀, the biological half-life of the compound, the age, size, and weight of the patient, and the disorder associated with the patient. The importance of these and other factors are well known to those of ordinary skill in the art. Generally, a dose will be between about 0.01 and 50 mg/kg, preferably 0.1 and 20 mg/kg of the patient being treated. Multiple doses may be used.

### EXAMPLES

A number of examples involved in the present invention are described below. In most cases, alternative techniques could also be used. The examples are intended to be illustrative and are not limiting or restrictive to the scope of the invention.

### Example 1

### Plasmid Construction

Human SF-1 and LRH-1 constructs were obtained by PCR amplification of cDNA (BD Biosciences). For E. coli expression the SF-1 G219-T461 insert was cloned into a modified pET vector (Novagen) encoding an N-terminal hexa-HIS tag, cleavable using TEV protease. The SF-1 LBD primer containing a BamHI cloning site and a TEV protease recognition site before residue G219 was:

(SEQ ID NO:_)

5′-GCTGGATCCGAAAACCTGTACTTCCAGGGAGGCCCCAACGTGCCT.

The non-coding strand primer, adding a stop codon and a SalI cloning site, was

(SEQ ID NO:_)

5′-GGATCCATGTCGACTCAAGTCTGCTTGGCTTGCAGCATTT.

An analogous strategy was used for expression of the LRH-1 S25 1 -A495 (see below, SEQ ID NO:______) using the coding-strand primer,

(SEQ ID NO:_)

5′-GCTGGATCCGAAAACCTGTACTTCCAGGGTTCTCCAGCAAGCATCCC
ACAT,

and the non-coding strand primer,

(SEQ ID NO:_)

5′-GTTCTTGTCGACTTATGCTCTTTTGGCATGCAAC.

From structure-based alignment with the mouse LRH-1 structure (1PK5) it was obvious that human SF-1 would have surface-exposed Cys residues at positions 247 and 412. For crystallography of SF-1 these Cys were removed by mutagenesis of the SF-1 DNA using Quick-change protocols (Stratagene) with complementary primers (see below, SEQ ID NO______). The coding-strand primers used were:

| | |
|---|---|
| SF-1-C247S: | |
| 5′-CGCATCTTGGGCTCTCTGCAGGAGCCCAC | (SEQ ID NO:_) |
| SF-1-C412S: | |
| 5′-CACTACCCGCACTCCGGGGACAAATTCC. | (SEQ ID NO:_) |

For analysis in mammalian cell culture, transient transfection vectors encoding the LBDs of SF-1 and LRH-1 were cloned as fusion proteins with the GAL4 DBD into a modified SG5-GAL4 vector. The SF-1 G219-T461 LBD primer containing an NdeI cloning site before residue G219 was:

| | |
|---|---|
| 5′-GTTCTTCATATGGGAGGCCCCAACGTGCCT. | (SEQ ID NO:_) |

The LRH-1 S251-A495 LBD primer containing an NdeI site before S251 was

| | |
|---|---|
| 5′-GTTCTTCATATGTCTCCAGCAAGCATCCCACAT. | (SEQ ID NO:_) |

Coding-strand primers for mutations of SF-1 and LRH-1 to test ligand binding and coactivator binding using Quick-change protocols were:

| | |
|---|---|
| SF-1 L245K | |
| 5′-CGGGCCCGCATCAAGGGCTGCCTGCAG | (SEQ ID NO:_) |
| SF-1 A269F | |
| 5′-CTCCTGTGCAGAATGTTCGACCAGACCTTC | (SEQ ID NO:_) |
| SF-1 E332A | |
| 5′-GGCAGGAGGTGGCACTGACCACAGTGG | (SEQ ID NO:_) |
| SF-1 G340E | |
| 5′-CACAGTGGCCACCCAGGCGGAGTCGCTGCTGCACAGC | (SEQ ID NO:_) |
| SF-1 L344F | |
| 5′-GCGGGCTCGCTGTTCCACAGCCTGGTGTTG | (SEQ ID NO:_) |
| SF-1 A433F | |
| 5′-CCTGAGCATGCAGTTCAAGGAGTACCTGTAC | (SEQ ID NO:_) |
| SF-1 Y436M | |
| 5′-GCAGGCCAAGGAGATGCTGTACCACAAGC | (SEQ ID NO:_) |
| SF-1 K440M | |
| 5′-GTACCTGTACCACATGCACCTGGGCAAC | (SEQ ID NO:_) |
| SF-1 Y436FK440M | |
| 5′-GCAGGCCAAGGAGATGCTGTACCACATGCACCTGGGCAAC | (SEQ ID NO:_) |
| SF-1 Y436FK440A | |
| 5′-GCAGGCCAAGGAGTTCCTGTACCACGCGCACCTGGGCAAC | (SEQ ID NO:_) |
| SF-1 E454K | |
| 5′-GCAACAACCTGCTCATCAAGATGCTGCAAGCCAAG | (SEQ ID NO:_) |
| LRH-1 M277K | |
| 5′-GTCCAGGCTAAAATCAAGGCCTATTTGCAGC | (SEQ ID NO:_) |
| LRH-1 L298Y | |
| 5′-GAGCACCTTTGGGTACATGTGCAAAATGGCAG | (SEQ ID NO:_) |
| LRH-1 A303F | |
| 5′-CTTATGTGCAAAATGTTCGATCAAACTCTCTTC | (SEQ ID NO:_) |
| LRH-1 A303M | |
| 5′-CTTATGTGCAAAATGATGGATCAAACTCTCTTC | (SEQ ID NO:_) |
| LRH-1 D366A | |
| 5′-CTGGGCAACAAGTGGCATATTCCATAATAGCATC | (SEQ ID NO:_) |
| LRH-1 I369Y | |
| 5′-CAAGTGGACTATTCCTACATAGCATCACAAGC | (SEQ ID NO:_) |
| LRH-1 L378F | |
| 5′-GCCGGAGCCACCTTCAACAACCTCATGAG | (SEQ ID NO:_) |
| LRH-1 A467F | |
| 5′-CCATCAGTATGCAGTTCGAAGAATACCTCTAC | (SEQ ID NO:_) |
| LRH-1 A467M | |
| 5′-CCATCAGTATGCAGATGGAAGAATACCTCTAC | (SEQ ID NO:_) |
| LRH-1 Y470FK474A | |
| 5′-GCAGGCTGAAGAATTCCTCTACTACGCGCACCTGAACGG | (SEQ ID NO:_) |
| LRH-1 E488K | |
| 5′-CTATAATAACCTTCTCATTAAGATGTTGCATGCCAAAAG | (SEQ ID NO:_) |

E. coli expression vectors for GST fusion proteins with SRC-1 (residues M595-Q780, containing NR-boxes I, II and III) were made as described (Marimuthu et al., Mol. Endocrinol., 2002, 16:271-86) except a modified pGEX-2T vector (Amersham) was engineered to encode a C-terminal fusion peptide,

| | |
|---|---|
| VDLNDIFEAQKIEWHR, | (SEQ ID NO:_) |

with a biotinylation site (Kim & McHenry, J. Biol. Chem., 1996, 271:20690-20698.) The insert encoding a NR-binding site from the coactivator TReP (Gizard et al., J. Biol. Chem., 2002, 277, 39144-39155), M173-P192, encoding residues

| | |
|---|---|
| MDGAPDSALRQLLSQKPMEP | (SEQ ID NO:_) |

was engineered by gene synthesis, and cloned into the N-terminal GST/C-terminal biotinylation site vector. All constructs were sequenced (DavisSequencing, Inc.).

SF-1 G219-T461 with Cys 247 and 412 Removed:

P1098. pET-SPEC SF1 G219-T461-X C247S, C412S

taatacgactcactataggggaattgt

```
gagcggataacaattcccctctagaaataattttgtttaactttaagaaggagatatacc
atgaaaaaaggtcaccaccatcaccatcacggatccgaaaacctgtacttccagggaggc
  M K K G H H H H H H G S E N L Y F Q G G
cccaacgtgcctgagctcatcctgcagctgctgcagctggagccggatgaggaccaggtg
  P N V P E L I L Q L L Q L E P D E D Q V
cgggcccgcatcttgggctctctgcaggagcccaccaaaagccgccccgaccagccggcg
  R A R I L G S L Q E P T K S R P D Q P A
gccttcggcctcctgtgcagaatggccgaccagaccttcatctccatcgtggactgggca
  A F G L L C R M A D Q T F I S I V D W A
cgcaggtgcatggtcttcaaggagctggaggtggccgaccagatgacgctgctgcagaac
  R R C M V F K E L E V A D Q M T L L Q N
tgctggagcgagctgctggtgttcgaccacatctaccgccaggtccagcacggcaaggag
  C W S E L L V F D H I Y R Q V Q H G K E
ggcagcatcctgctggtcaccgggcaggaggtggagctgaccacagtggccacccaggcg
  G S I L L V T G Q E V E L T T V A T Q A
ggctcgctgctgcacagcctggtgttgcgggcgcaggagctggtgctgcagctgcttgcg
  G S L L H S L V L R A Q E L V L Q L L A
ctgcagctggaccggcaggagtttgtctgcctcaagttcatcatcctcttcagcctggat
  L Q L D R Q E F V C L K F I I L F S L D
ttgaagttcctgaataaccacatcctggtgaaagacgctcaggagaaggccaacgccgcc
  L K F L N N H I L V K D A Q E K A N A A
ctgcttgactacaccctgtgccactacccgcactccggggacaaattccagcagctactg
  L L D Y T L C H Y P H S G D K F Q Q L L
ctgtgcctggtggaggtgcgggccctgagcatgcaggccaaggagtacctgtaccacaag
  L C L V E V R A L S M Q A K E Y L Y H K
cacctgggcaacgagatgccccgcaacaacctgctcatcgaaatgctgcaagccaagcag
  H L G N E M P R N N L L I E M L Q A K Q
acttgagtcgaccaccaccaccaccaccactgagatccggctggccctactggccgaaag
  T -
gaattcgaggccagcagggccaccgctgagcaataactagcataaccccttggggcctct
aaacgggtcttgaggggttttttg
```

Nucleic acid(SEQ ID NO: ______)

Encoded protein (SEQ ID NO: ______)

LRH-1 S251-A495 with Cys 247 and 412 Removed:

P1515. pET-SPEC LRH-1 GS251-A495-X

taatacgactcactataggggaattgt

```
gagcggataacaattcccctctagaaataattttgtttaactttaagaaggagatatacc
atgaaaaaaggtcaccaccatcaccatcacggatccgaaaacctgtacttccagggttct
  M K K G H H H H H H G S E N L Y F Q G S
ccagcaagcatcccacatctgatactggaacttttgaagtgtgagccagatgagcctcaa
  P A S I P H L I L E L L K C E P D E P Q
gtccaggctaaaatcatggcctatttgcagcaagagcaggctaaccgaagcaagcacgaa
  V Q A K I M A Y L Q Q E Q A N R S K H E
aagctgagcacctttgggcttatgtgcaaaatggcagatcaaactctcttctccattgtc
  K L S T F G L M C K M A D Q T L F S I V
gagtgggccaggagtagtatcttcttcagagaacttaaggttgatgaccaaatgaagctg
  E W A R S S I F F R E L K V D D Q M K L
cttcagaactgctggagtgagctcttaatcctcgaccacatttaccgacaagtggtacat
  L Q N C W S E L L I L D H I Y R Q V V H
ggaaaggaaggatccatcttcctggttactgggcaacaagtggactattccataatagca
  G K E G S I F L V T G Q Q V D Y S I I A
tcacaagccggagccaccctcaacaacctcatgagtcatgcacaggagttagtggcaaaa
  S Q A G A T L N N L M S H A Q E L V A K
cttcgttctctccagtttgatcaacgagagttcgtatgtctgaaattcttggtgctcttt
  L R S L Q F D Q R E F V C L K F L V L F
agtttagatgtcaaaaaccttgaaaacttccagctggtagaaggtgtccaggaacaagtc
  S L D V K N L E N F Q L V E G V Q E Q V
aatgccgccctgctggactacacaatgtgtaactacccgcagcagacagagaaatttgga
  N A A L L D Y T M C N Y P Q Q T E K F G
cagctacttcttcgactacccgaaatccgggccatcagtatgcaggctgaagaatacctc
  Q L L L R L P E I R A I S M Q A E E Y L
tactacaagcacctgaacggggatgtgccctataataaccttctcattgaaatgttgcat
  Y Y K H L N G D V P Y N N L L I E M L H
gccaaaagagcataagtcgaccaccaccaccaccaccactgagatccggctggccctact
  A K R A -
ggccgaaaggaattcgaggccagcagggccaccgctgagcaataactagcataacccctt
ggggcctctaaacgggtcttgaggggttttttg
```

Nucleic acid(SEQ ID NO: ______)

Encoded protein (SEQ ID NO: ______)

### Example 2

### Protein Expression and Purification

The SF-1 LBD (G219-T416 with C247S/C412S mutations) and the LRH-1 LBD (S251 -A495) used for crystallography were produced as TEV-cleavable N-terminally HIS-tagged proteins in E. coli strain BL21(DE3) RIL (Stratagene). Single colonies were grown for 4 hrs at 37° C. in 2 separate 200 mL Luria broth (LB) media containing kanamycin (30 μg/mL) and chloramphenicol (15 μg/mL). 400 mL culture was transferred to a 45 L Bioreactor containing 30 L Terrific Broth (TB) media also supplemented with kanamycin and chloramphenicol. Cultures were allowed to grow at 37° C. until reaching an OD₆₀₀ of 2.0-2.5 OD then grown at 20° C., with 0.5 mM IPTG added for continued growth for 15 hrs at 20° C. Cells were harvested using a continuous flow centrifuge and paste frozen at −80° C.

Cell pastes with SF-1 or LRH-1 were resuspended with 40 mL lysis buffer (50 mM Na/K Phosphate [pH 8.0], 250 mM NaCl, 5% glycerol) per liter of cells, and lysed using a microfluidizer (Microfluidics M-110H) at 18,000 psi. Lysate was clarified by centrifugation at 15,000 g at 4° C. for 2 hrs. Imidazole was added to the clarified lysate to a final concentration of 15 mM, and then loaded onto a 50 ml Ni-Chelating Sepharose (AP Biotech) column. The column was washed with 500 mL of buffer A (20 mM HEPES [pH8.0], 250 mM NaCl, 5% glycerol) containing 15 mM imidazole, and eluted with a 100 mL gradient to 100% buffer B (20 mM HEPES [pH8.0], 250 mM imidazole, 250 mM NaCl, 5% glycerol). Eluted LBDs were diluted six-fold with buffer C (20 mM Tris [pH 8.0]) and loaded onto a 75 mL Source 30Q (AP Biotech) column. The column was washed with 100 mL buffer C containing 20 mM NaCl and eluted with a fifteen column volume linear gradient from 2 to 25% buffer D (20 mM Tris [pH 8.0], 1 M NaCl). The LBD proteins, which eluted between 50 mM and 150 mM NaCl, were analyzed using native and SDS-PAGE, and tested for coactivator-binding activity. Pooled fractions were incubated with TEV protease at 50 μg/mg overnight at 4° C. for removal of the N-terminal tag. The sequence removed is:

| | |
|---|---|
| MKKGHHHHHHGSENLYFQ | (SEQ ID NO:_) |

The cleaved protein was re-purified using a Source30Q column, and eluted with an eight column volume gradient from 2 to 25% buffer D. At this stage, the proteins were >95% pure as determined by SDS-PAGE analysis. Prior to concentration, beta-mercaptoethanol was added to 14 mM final concentration, and the proteins concentrated to 20 mg/mL and stored at −80° C.

Coactivator N-terminal GST/C-terminal biotinylation site fusion proteins were produced in E. coli strain BL21(DE3) RIL (Stratagene). Shaker cultures (750 ml 2× LB) were grown at 37° C. until an OD₆₀₀ of 1.2. Then, 0.5 mM IPTG was added and cultures were cooled to 15° C. with continued shaking overnight. Cells were harvested by centrifugation, frozen in liquid N₂ and stored at −80° C. Cell pastes (5 gm) were suspended in 50 mL extraction buffer (50 mM Tris pH 8.0,250 mM NaCl, 0.1% Triton X-100). Lysozyme (0.5 mL of 20 mg/mL, Sigma) was added and left on ice 15-30 min., followed by sonication (1.5 min on ice) using flat-tip probe and setting 6 of model 550-sonic dismembranator (Fisher). The prep was checked for loss of DNA viscosity, then centrifuged at 17,000 rpm for 30 min. at 4° C. in a SA-600 rotor (Beckman). Supernatant was recovered and mixed with 0.5 mL buffer-washed slurry of Glutathione-Sepharose beads (Amersham) continuously for 1 hr at 4° C. Beads were centrifuged at low speed and washed once with 20 mL extraction buffer, and twice with 50 mM Tris pH 8.0. GST protein was recovered by elution with 3-5 ml elution buffer (50 mM Tris pH 8.0, 6.5 mg/ml glutathione (Sigma).

For co-expression studies, the ampicillin-resistant GST-coactivator fusion plasmids were co-introduced with the kanamycin-resistant HIS-tagged LRH-1 or SF-1 plasmids. Growth and extraction was the same as for GST-tagged coactivators, above. To the centrifuged prep from 750 mL culture was added imidazole to a final 10 mM, and 1.0 mL buffer-washed slurry of Talon cobalt affinity resin (BD Biosciences), stirring continuously for 1 hr at 4° C. Beads were centrifuged at low speed and washed once with 20 mL extraction buffer containing 10 mM imidazole, and twice with cobalt wash buffer (20 mM Tris pH 8.0, 100 mM NaCl, 10% glycerol) also with 10 mM imidazole. HIS-tagged protein was recovered by elution with 3-5 ml cobalt wash buffer with 200 mM imidazole.

For liposome washing of HIS-tagged SF-1 protein, 20 mg was extracted from a 750 mL culture, bound to cobalt affinity resin, and washed as above. While remaining bound to the resin, two sequential 30 minute, 5 mL washes in cobalt wash buffer containing sonicated 100 μM 1,2-didodecanoyl-sn-glycero-3-phosphocholine (Sigma) were applied, followed by two final washes in cobalt wash buffer. The HIS-tagged protein was recovered in 3 mL cobalt wash buffer with 200 mM imidazole.

### Example 3

### Crystallization

Initial crystallization of human SF-1 and LRH-1 were observed in sparse-matrix screens using Hampton Index screen kits (Hampton Research). Human SF-1 protein was diluted to 15 mg/ml in 20 mM Tris-HCl, pH 8.0, 100 mM NaCl, 10 mM DTT with a 2× molar excess of the peptides NCOA1 (SRC-1) NID-2

| | |
|---|---|
| CPSSHSSLTERHKILHRLLQEGSPS | (SEQ ID NO:_) |

and/or NCOA2 (TIF2, GRIP1) NID-3

| | |
|---|---|
| KENALLRYLLDKD. | (SEQ ID NO:_) |

Crystals were grown by sitting drop vapor diffusion at 4° C., mixing equal volumes of protein/peptide sample with reservoir solution containing 18% polyethylene glycol (PEG) 3350, 0.2M ammonium sulfate, 0.1M BisTris pH 5.5, and 2.5% sucrose. Crystals grew to a size of 0.6 mm×0.3 mm×0.3 mm in 5-8 days. For cryo-protection sucrose was added to SF-1 crystals prior to freezing.

Human LRH-1 protein was diluted to 10 mg/ml in 20 mMTris/HCl, pH 7.5, 62 mM NaCl, 100 mM ammonium acetate, 2 mM CHAPS with 2× molar excess of the peptide NCOA2 NID-3

| | |
|---|---|
| KENALLRYLLDKD. | (SEQ ID NO:_) |

Crystals were grown by sitting drop vapor diffusion at 20° C., mixing equal volumes of protein/peptide sample with reservoir solution containing 0.9M NaH₂PO₄, 0.1 M K₂HPO₄ (Hampton Index screen #17). Crystals grew to a size of 0.13 mm×0.03 mm×0.03 mm in 2 weeks. Glycerol was used for cryo-protection.

### Example 4

### Crystal Data Collection and Structure Determination

The X-ray diffraction data of both human SF-1 and human LRH-1 were collected at the Advanced Light Source (ALS) beam line 8.3.1 using a Quantum 210 CCD detector. Data collection was performed under cryogenic temperature. The diffraction data were integrated and scaled using programs Mosflm and SCALA (Table 1). (Leslie, Acta Crystallogr. D Biol Crystallogr., 1999, 55 (Pt 10):1696-1702.)

To solve the SF-1 structure, a homology model was generated based on the crystal structure of mouse LRH-1 (1PK5). (Sablin et al., Mol. Cell, 2003, 11:1575-1585.) Molecular replacement of the data up to 3.5 Å was carried out using EPMR (Kissinger et al., Acta Crystallogr. D Biol Crystallogr., 1999, 55 (Pt 2):484-91) obtaining a solution in space group P3₁21. Two molecules related by non-crystallographic symmetry were determined in each asymmetric unit. The electron density map calculated with the initial phases revealed the majority of the structure. An initial model was obtained manually using program O. (Jones et al., Acta Crystallogr A, 1991, 47 ( Pt 2):110-9.) The initial model was then subject to refinement using program CNX (Brunger et al., Acta Crystallogr D Biol Crystallogr., 1998, 54 (Pt 5):905-21) with least square minimization on the maximum likelihood target functions, simulated annealing and torsion angle dynamics. Subsequent interactive model building and refinement were performed against 2.1 Å data with least square refinement, individual B-factor refinement, and TLS refinement using programs CNX and REFMAC5. (Brunger et al., Acta Crystallogr D Biol Crystallogr., 1998, 54 (Pt 5):905-21.) Well-defined election density indicated one NCOA2 NID-3 peptide bound to the surface and the unexpected PE ligand bound inside the ligand pocket.

The human LRH-1 structure determination and refinement was similar to that for SF-1. A homology model was generated based on the crystal structure of mouse LRH-1 (1PK5). (Sablin et al., Mol. Cell, 2003, 11, 1575-85.) It was then used as the search model for molecular replacement using program EPMR. (Kissinger et al., Acta Crystallogr D Biol Crystallogr., 1999, 55 ( Pt 2):484-91.) The crystal is in space group P2₁2₁2₁ with one molecule in each asymmetric unit. The initial molecular replacement solution was then subject to iterative refinement against data up to 2.5 Å. At a late stage of refinement, some electron density appeared in the ligand binding pocket representing a phospholipid molecule. The shape of the electron density suggested the structure of a phosphatidylglycerol-phosphoglycerol, confirmed by further refinement. NCOA2 NID-3 peptide was found to bind at two sites on the molecular surface.

### Example 5

### Biochemical Protein Interaction Assay

The Alpha Screen Histidine detection (Nickel chelate) kit (Perkin Elmer) was used to detect binding between His-tagged SF-1 LBD and biotinylated GST-SRC-1 fragments. The assay was performed in Costar 384-well white polystyrene plates (Coming Inc.) in a total volume of 20 μL using buffer containing 50 mM Bis-tris HCl (pH 7.5), 50 mM KCl, 0.05% Tween 20, 1 mM DTT, 0.1% BSA. Reactions were initiated in 15 μL containing 50 nM His-tagged SF-1 receptor and 50 nM biotin-tagged SRC-1 fragment. Phospholipid was included as indicated. PE 18:3 (1,2-Dilinolenoyl-sn-glycero-3-phosphoethanolamine) was from Avanti Polar Lipids. The plate was sealed and incubated at room temp for 2 hours. After incubation, 5 μL containing streptavidin donor beads (15 μg/ml) and Ni-chelate acceptor beads (15 μg/ml) was added from the Nickel chelate kit. Plates were resealed and incubated in the dark for 2 hours at room temperature and then read in a Fusion Alpha reader set at a read time of 1 s/well. Data analysis was done using GraphPad Prism (GraphPad Software, Inc.).

### Example 6

### Cell Culture

HEK293T cells were cultured at 37° C. in Dulbecco's modified Eagle's medium(DMEM) with penicillin(100 U/ml), streptomycin (100 U/ml) and 10% heat-inactivated fetal calf serum (Invitrogen). For transient transfection HEK293T cells were grown to 80% confluency in 96-well plates, and medium exchanged for 100 μl serum-free medium before addition of 100 ng pSG-GAL4-SF-1 -LBD or pSG-GAL4-LRH-1 -LBD expression vector, 40 ng pFR-Luc reporter gene (Stratagene), and 12 ng pRL-TK transfection control plasmids (Promega) mixed with 0.5 μl Metafectene (Biontex). After 4 hours serum-containing medium was added. After 24 hrs medium was removed and cells were lysed in Renilla luciferase assay lysis buffer (Promega). Firefly luciferase was measured using Luciferase Reporter Gene Assay kit (Roche) and Renilla luciferase was measured using Renilla Luciferase Assay System (Promega).

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made in the method for identifying modulators and/or various methods of administration can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

TABLE 1
Statistics of crystallographic data and refinement.

| Crystallization and data collection | | | Refinement | | |
|---|---|---|---|---|---|
| | SF-1 | LRH-1 | | SF-1 | LRH-1 |
| Unit cell | a = b = 73.6, | a = 61.0, b = 67.0, | Resolution | 50-2.1 | 50-2.5 |
| dimensions (Å) | c = 195.7 | c = 78.2 | range (Å) | | |
| Space group | P3₁21 | P2₁2₁2₁ | σ cut off | none | none |
| Solvent content | 49% | 53% | Total non- | 4342 | 2172 |
| | | | hydrogen | | |
| | | | atoms | | |
| Resolution range | 50-2.1 | 50-2.5 | Average B | 22.6 | 33.6 |
| (Å) | | | factor (Å²), | | |
| | | | Main chain | | |
| Unique reflections | 36333 | 10899 | Average B | 24.0 | 34.2 |
| | | | factor (Å²), | | |
| | | | Side chain | | |
| Data redundancy | 4.2 | 4.6 | Average B | 24.89 | 32.2 |
| | | | factor (Å²), | | |
| | | | Solvent | | |
| Completeness (%) | 98.7 | 99.4 | R_{cryst}/R_{free} | 21.6/ | 23.9/ |
| | | | (%)^{b} | 26.5 | 28.1 |
| <I/σ(I)> | 6.9 | 10.0 | r.m.s.d.^{c} | 0.012 | 0.008 |
| | | | bond | | |
| | | | lengths (Å) | | |
| Rsym (%)^{a} | 11.2 | 4.9 | r.m.s.d.^{c} | 1.449 | 1.034 |
| | | | bond | | |
| | | | angles (°) | | |

^{a}R_{sym} = Σ|I_{avg} − Iⱼ|/ΣIⱼ.
^{b}R_{cryst} = Σ|Fₒ − F_{c}|/ΣFₒ, where Fₒ and F_{c} are observed and calculated structure factors, respectively, R_{free} was calculated from a randomly chosen 5% of reflections excluded form the refinement, and R_{cryst} was calculated from the remaining 95% of reflections.
r.m.s.d. is the root-mean-square deviation from ideal geometry. Numbers in parentheses are for the highest resolution shell.

TABLE 2
Atomic coordinates for SF1 crystal

| HEADER | --- XX-XXX-XX WWAN | |
|---|---|---|
| COMPND | SF-1, APO, with phospholipid | |
| REMARK | 3 | |
| REMARK | 3 | REFINEMENT. |

| | | | |
|---|---|---|---|
| REMARK | 3 | PROGRAM | : REFMAC 5.1.25 |
| REMARK | 3 | AUTHORS | : MURSHUDOV, VAGIN, DODSON |
| REMARK | 3 | | |

| | | |
|---|---|---|
| REMARK | 3 | REFINEMENT TARGET: MAXIMUM LIKELIHOOD |
| REMARK | 3 | |

| | | |
|---|---|---|
| REMARK | 3 | DATA USED IN REFINEMENT. |

| | | | | |
|---|---|---|---|---|
| REMARK | 3 | RESOLUTION RANGE HIGH | (ANGSTROMS) : | 2.10 |
| REMARK | 3 | RESOLUTION RANGE LOW | (ANGSTROMS) : | 50.00 |
| REMARK | 3 | DATA CUTOFF | (SIGMA(F)) : | NONE |
| REMARK | 3 | COMPLETENESS FOR RANGE | (%) : | 99.31 |
| REMARK | 3 | NUMBER OF REFLECTIONS | : | 34644 |
| REMARK | 3 | | | |

| | | |
|---|---|---|
| REMARK | 3 | FIT TO DATA USED IN REFINEMENT. |

| REMARK | 3 | CROSS-VALIDATION METHOD | | : | THROUGHOUT |
|---|---|---|---|---|---|
| REMARK | 3 | FREE R VALUE TEST SET SELECTION | | : | RANDOM |
| REMARK | 3 | R VALUE | (WORKING + TEST SET) | : | 0.21823 |
| REMARK | 3 | R VALUE | (WORKING SET) | : | 0.21597 |

| REMARK | 3 | FREE R VALUE | | : | 0.26532 |
|---|---|---|---|---|---|
| REMARK | 3 | FREE R VALUE TEST SET SIZE | (%) | : | 4.3 |
| REMARK | 3 | FREE R VALUE TEST SET COUNT | | : | 1565 |
| REMARK | 3 | | | | |

| | | |
|---|---|---|
| REMARK | 3 | FIT IN THE HIGHEST RESOLUTION BIN. |

| REMARK | 3 | TOTAL NUMBER OF BINS USED | | : | 20 |
|---|---|---|---|---|---|
| REMARK | 3 | BIN RESOLUTION RANGE HIGH | | : | 2.100 |
| REMARK | 3 | BIN RESOLUTION RANGE LOW | | : | 2.155 |
| REMARK | 3 | REFLECTION IN BIN | (WORKING SET) | : | 2494 |
| REMARK | 3 | BIN R VALUE | (WORKING SET) | : | 0.335 |

| | | | | |
|---|---|---|---|---|
| REMARK | 3 | BIN FREE R VALUE SET COUNT | : | 0 |
| REMARK | 3 | BIN FREE R VALUE | : | −999.000 |
| REMARK | 3 | | | |

| | | |
|---|---|---|
| REMARK | 3 | NUMBER OF NON-HYDROGEN ATOMS USED IN REFINEMENT. |

| | | | | |
|---|---|---|---|---|
| REMARK | 3 | ALL ATOMS | : | 4324 |
| REMARK | 3 | | | |

| | | |
|---|---|---|
| REMARK | 3 | B VALUES. |

| | | | | |
|---|---|---|---|---|
| REMARK | 3 | FROM WILSON PLOT | (A**2) : | NULL |
| REMARK | 3 | MEAN B VALUE | (OVERALL, A**2) : | 21.368 |

| | | |
|---|---|---|
| REMARK | 3 | OVERALL ANISOTROPIC B VALUE. |

| | | | |
|---|---|---|---|
| REMARK | 3 | B11 (A**2) : | 1.34 |
| REMARK | 3 | B22 (A**2) : | 1.34 |
| REMARK | 3 | B33 (A**2) : | −2.01 |
| REMARK | 3 | B12 (A**2) : | 0.67 |
| REMARK | 3 | B13 (A**2) : | 0.00 |
| REMARK | 3 | B23 (A**2) : | 0.00 |
| REMARK | 3 | | |

| | | |
|---|---|---|
| REMARK | 3 | ESTIMATED OVERALL COORDINATE ERROR. |

| | | | | |
|---|---|---|---|---|
| REMARK | 3 | ESU BASED ON R VALUE | (A) : | 0.230 |
| REMARK | 3 | ESU BASED ON FREE R VALUE | (A) : | 0.200 |
| REMARK | 3 | ESU BASED ON MAXIMUM LIKELIHOOD | (A) : | 0.205 |
| REMARK | 3 | ESU FOR B VALUES BASED ON MAXIMUM LIKELIHOOD | (A**2) : | 8.778 |
| REMARK | 3 | | | |

| | | |
|---|---|---|
| REMARK | 3 | CORRELATION COEFFICIENTS. |

| | | | |
|---|---|---|---|
| REMARK | 3 | CORRELATION COEFFICIENT FO-FC | : 0.951 |
| REMARK | 3 | CORRELATION COEFFICIENT FO-FC FREE | : 0.926 |
| REMARK | 3 | | |

| | | | |
|---|---|---|---|
| REMARK | 3 | RMS DEVIATIONS FROM IDEAL VALUES | COUNT RMS WEIGHT |

| | | | | |
|---|---|---|---|---|
| REMARK | 3 | BOND LENGTHS REFINED ATOMS | (A): | 4150 ; 0.014 ; 0.021 |
| REMARK | 3 | BOND LENGTHS OTHERS | (A): | 3959 ; 0.002 ; 0.020 |
| REMARK | 3 | BOND ANGLES REFINED ATOMS | (DEGREES): | 5585 ; 1.449 ; 1.999 |
| REMARK | 3 | BOND ANGLES OTHERS | (DEGREES): | 9218 ; 0.870 ; 3.000 |
| REMARK | 3 | TORSION ANGLES, PERIOD 1 | (DEGREES): | 489 ; 6.106 ; 5.000 |
| REMARK | 3 | CHIRAL-CENTER RESTRAINTS | (A**3): | 648 ; 0.083 ; 0.200 |
| REMARK | 3 | GENERAL PLANES REFINED ATOMS | (A): | 4450 ; 0.004 ; 0.020 |
| REMARK | 3 | GENERAL PLANES OTHERS | (A): | 771 ; 0.003 ; 0.020 |
| REMARK | 3 | NON-BONDED CONTACTS REFINED ATOMS | (A): | 1036 ; 0.204 ; 0.200 |
| REMARK | 3 | NON-BONDED CONTACTS OTHERS | (A): | 4476 ; 0.222 ; 0.200 |
| REMARK | 3 | NON-BONDED TORSION OTHERS | (A): | 2537 ; 0.095 ; 0.200 |
| REMARK | 3 | H-BOND (X...Y) REFINED ATOMS | (A): | 190 ; 0.216 ; 0.200 |
| REMARK | 3 | SYMMETRY VDW REFINED ATOMS | (A): | 19 ; 0.195 ; 0.200 |
| REMARK | 3 | SYMMETRY VDW OTHERS | (A): | 74 ; 0.202 ; 0.200 |
| REMARK | 3 | SYMMETRY H-BOND REFINED ATOMS | (A): | 14 ; 0.283 ; 0.200 |
| REMARK | 3 | | | |

| | | | |
|---|---|---|---|
| REMARK | 3 | ISOTROPIC THERMAL FACTOR RESTRAINTS. | COUNT RMS WEIGHT |

| | | | | |
|---|---|---|---|---|
| REMARK | 3 | MAIN-CHAIN BOND REFINED ATOMS | (A**2): | 2472 ; 0.483 ; 1.500 |
| REMARK | 3 | MAIN-CHAIN ANGLE REFINED ATOMS | (A**2): | 3971 ; 0.939 ; 2.000 |
| REMARK | 3 | SIDE-CHAIN BOND REFINED ATOMS | (A**2): | 1678 ; 1.532 ; 3.000 |
| REMARK | 3 | SIDE-CHAIN ANGLE REFINED ATOMS | (A**2): | 1614 ; 2.579 ; 4.500 |
| REMARK | 3 | | | |

| | | |
|---|---|---|
| REMARK | 3 | NCS RESTRAINTS STATISTICS |
| REMARK | 3 | NUMBER OF NCS GROUPS: NULL |
| REMARK | 3 | |
| REMARK | 3 | |
| REMARK | 3 | TLS DETAILS |

| | | |
|---|---|---|
| REMARK | 3 | NUMBER OF TLS GROUPS : 5 |
| REMARK | 3 | |
| REMARK | 3 | TLS GROUP: 1 |

| REMARK | 3 | NUMBER OF COMPONENTS GROUP: 4 | | | | | |
|---|---|---|---|---|---|---|---|
| REMARK | 3 | COMPONENTS | C | SSSEQI | TO | C | SSSEQI |
| REMARK | 3 | RESIDUE RANGE: | A | 221 | | A | 248 |
| REMARK | 3 | RESIDUE RANGE: | A | 256 | | A | 460 |
| REMARK | 3 | RESIDUE RANGE: | L | 1 | | L | 1 |
| REMARK | 3 | RESIDUE RANGE: | S | 1 | | S | 96 |

| | | | | | |
|---|---|---|---|---|---|
| REMARK | 3 | ORIGIN FOR THE GROUP (A): | 4.6333 | 15.7404 | 77.4678 |

| | | | | | | | |
|---|---|---|---|---|---|---|---|
| REMARK | 3 | T TENSOR | | | | | |
| REMARK | 3 | T11: | 0.1475 | T22: | 0.0430 | | |
| REMARK | 3 | T33: | 0.0966 | T12: | 0.0424 | | |
| REMARK | 3 | T13: | 0.0394 | T23: | −0.0073 | | |
| REMARK | 3 | L TENSOR | | | | | |
| REMARK | 3 | L11: | 3.9798 | L22: | 1.1850 | | |
| REMARK | 3 | L33: | 3.8661 | L12: | 1.0540 | | |
| REMARK | 3 | L13: | −1.4853 | L23: | −0.7088 | | |
| REMARK | 3 | S TENSOR | | | | | |
| REMARK | 3 | S11: | −0.2073 | S12: | 0.0305 | S13: | −0.2304 |
| REMARK | 3 | S21: | −0.1365 | S22: | 0.1174 | S23: | −0.1467 |
| REMARK | 3 | S31: | 0.4625 | S32: | 0.1031 | S33: | 0.0899 |
| REMARK | 3 | | | | | | |

| | | |
|---|---|---|
| REMARK | 3 | TLS GROUP: 2 |

| REMARK | 3 | NUMBER OF COMPONENTS GROUP: 3 | | | | | |
|---|---|---|---|---|---|---|---|
| REMARK | 3 | COMPONENTS | C | SSSEQI | TO | C | SSSEQI |
| REMARK | 3 | RESIDUE RANGE: | B | 221 | | B | 459 |
| REMARK | 3 | RESIDUE RANGE: | L | 2 | | L | 2 |
| REMARK | 3 | RESIDUE RANGE: | S | 97 | | S | 189 |

| | | | | | |
|---|---|---|---|---|---|
| REMARK | 3 | ORIGIN FOR THE GROUP (A): | 13.8346 | −26.5101 | 96.2497 |

| | | | | | | | |
|---|---|---|---|---|---|---|---|
| REMARK | 3 | T TENSOR | | | | | |
| REMARK | 3 | T11: | 0.0364 | T22: | 0.0642 | | |
| REMARK | 3 | T33: | 0.1365 | T12: | −0.0393 | | |
| REMARK | 3 | T13: | −0.0407 | T23: | 0.0165 | | |
| REMARK | 3 | L TENSOR | | | | | |
| REMARK | 3 | L11: | 2.3171 | L22: | 2.3418 | | |
| REMARK | 3 | L33: | 4.7606 | L12: | −0.1019 | | |
| REMARK | 3 | L13: | −0.9180 | L23: | 0.1451 | | |
| REMARK | 3 | S TENSOR | | | | | |
| REMARK | 3 | S11: | 0.0958 | S12: | −0.0670 | S13: | −0.0457 |
| REMARK | 3 | S21: | 0.1480 | S22: | −0.1934 | S23: | −0.0795 |
| REMARK | 3 | S31: | −0.1881 | S32: | 0.4297 | S33: | 0.0975 |
| REMARK | 3 | | | | | | |

| | | |
|---|---|---|
| REMARK | 3 | TLS GROUP: 3 |

| REMARK | 3 | NUMBER OF COMPONENTS GROUP: 1 | | | | | |
|---|---|---|---|---|---|---|---|
| REMARK | 3 | COMPONENTS | C | SSSEQI | TO | C | SSSEQI |
| REMARK | 3 | RESIDUE RANGE: | S | 190 | | S | 229 |

| | | | | | |
|---|---|---|---|---|---|
| REMARK | 3 | ORIGIN FOR THE GROUP (A): | 3.9384 | −7.1494 | 86.0037 |

| | | | | | | | |
|---|---|---|---|---|---|---|---|
| REMARK | 3 | T TENSOR | | | | | |
| REMARK | 3 | T11: | 0.1945 | T22: | 0.2987 | | |
| REMARK | 3 | T33: | 0.1919 | T12: | −0.0351 | | |
| REMARK | 3 | T13: | 0.0624 | T23: | −0.0477 | | |
| REMARK | 3 | L TENSOR | | | | | |
| REMARK | 3 | L11: | 0.1838 | L22: | 0.9466 | | |
| REMARK | 3 | L33: | 0.1083 | L12: | −0.1097 | | |
| REMARK | 3 | L13: | 0.2112 | L23: | −0.3093 | | |
| REMARK | 3 | S TENSOR | | | | | |
| REMARK | 3 | S11: | −0.0201 | S12: | 0.0012 | S13: | −0.0369 |
| REMARK | 3 | S21: | 0.0032 | S22: | 0.0667 | S23: | −0.1284 |
| REMARK | 3 | S31: | −0.0260 | S32: | −0.0310 | S33: | −0.0465 |
| REMARK | 3 | | | | | | |

| | | |
|---|---|---|
| REMARK | 3 | TLS GROUP: 4 |

| REMARK | 3 | NUMBER OF COMPONENTS GROUP: 2 | | | | | |
|---|---|---|---|---|---|---|---|
| REMARK | 3 | COMPONENTS | C | SSSEQI | TO | C | SSSEQI |
| REMARK | 3 | RESIDUE RANGE: | P | 741 | | P | 752 |
| REMARK | 3 | RESIDUE RANGE: | S | 230 | | S | 232 |

| | | | | | |
|---|---|---|---|---|---|
| REMARK | 3 | ORIGIN FOR THE GROUP (A): | 12.8434 | 22.0178 | 93.3912 |

| | | | | | | | |
|---|---|---|---|---|---|---|---|
| REMARK | 3 | T TENSOR | | | | | |
| REMARK | 3 | T11: | 0.1206 | T22: | 0.2030 | | |
| REMARK | 3 | T33: | 0.0632 | T12: | 0.0220 | | |
| REMARK | 3 | T13: | 0.0091 | T23: | −0.0144 | | |
| REMARK | 3 | L TENSOR | | | | | |
| REMARK | 3 | L11: | 28.8425 | L22: | 4.4555 | | |
| REMARK | 3 | L33: | 26.0428 | L12: | 7.0765 | | |
| REMARK | 3 | L13: | 1.3623 | L23: | −4.4463 | | |
| REMARK | 3 | S TENSOR | | | | | |
| REMARK | 3 | S11: | −0.3451 | S12: | 0.0365 | S13: | −0.0457 |
| REMARK | 3 | S21: | 0.2228 | S22: | 0.0521 | S23: | −0.7244 |
| REMARK | 3 | S31: | −0.4484 | S32: | 1.1557 | S33: | 0.2930 |
| REMARK | 3 | | | | | | |

| | | |
|---|---|---|
| REMARK | 3 | TLS GROUP: 5 |

| REMARK | 3 | NUMBER OF COMPONENTS GROUP: 2 | | | | | |
|---|---|---|---|---|---|---|---|
| REMARK | 3 | COMPONENTS | C | SSSEQI | TO | C | SSSEQI |
| REMARK | 3 | RESIDUE RANGE: | Q | 741 | | Q | 751 |
| REMARK | 3 | RESIDUE RANGE: | S | 233 | | S | 235 |

| | | | | | |
|---|---|---|---|---|---|
| REMARK | 3 | ORIGIN FOR THE GROUP (A): | 29.1754 | −18.1701 | 101.3310 |

| | | | | | | | |
|---|---|---|---|---|---|---|---|
| REMARK | 3 | T TENSOR | | | | | |
| REMARK | 3 | T11: | 0.5240 | T22: | 0.5317 | | |
| REMARK | 3 | T33: | 0.5700 | T12: | −0.3155 | | |
| REMARK | 3 | T13: | −0.0879 | T23: | 0.1024 | | |
| REMARK | 3 | L TENSOR | | | | | |
| REMARK | 3 | L11: | 20.5790 | L22: | −4.0154 | | |
| REMARK | 3 | L33: | 4.2051 | L12: | −1.1596 | | |
| REMARK | 3 | L13: | 6.8311 | L23: | −2.6023 | | |
| REMARK | 3 | S TENSOR | | | | | |
| REMARK | 3 | S11: | 0.2938 | S12: | −0.6226 | S13: | 1.0566 |
| REMARK | 3 | S21: | 0.3209 | S22: | −0.7442 | S23: | −1.7446 |
| REMARK | 3 | S31: | −0.1387 | S32: | 0.6812 | S33: | 0.4504 |
| REMARK | 3 | | | | | | |
| REMARK | 3 | | | | | | |

| | | |
|---|---|---|
| REMARK | 3 | BULK SOLVENT MODELLING. |

| REMARK | 3 | METHOD USED: BABINET MODEL WITH MASK | | |
|---|---|---|---|---|
| REMARK | 3 | PARAMETERS FOR MASK CALCULATION | | |
| REMARK | 3 | VDW PROBE RADIUS | : | 1.40 |
| REMARK | 3 | ION PROBE RADIUS | : | 0.80 |
| REMARK | 3 | SHRINKAGE RADIUS | : | 0.80 |
| REMARK | 3 | | | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |

| | | | |
|---|---|---|---|
| LINK | LEU A 248 | PRO A 256 | gap |

| | |
|---|---|
| CRYST1 | 73.601 73.601 195.678 90.00 90.00 120.00 P 31 2 1 0 |

| | |
|---|---|
| SCALE1 | 0.013587 0.007844 0.000000 0.00000 |
| SCALE2 | 0.000000 0.015689 0.000000 0.00000 |
| SCALE3 | 0.000000 0.000000 0.005110 0.00000 |

| | | | | | | | | | | | |
|---|---|---|---|---|---|---|---|---|---|---|---|
| ATOM | 1 | N | PRO | A | 221 | 19.749 | 11.027 | 56.510 | 1.00 | 31.78 | N |
| ATOM | 2 | CA | PRO | A | 221 | 20.828 | 10.210 | 57.147 | 1.00 | 31.53 | C |
| ATOM | 3 | CB | PRO | A | 221 | 21.683 | 9.741 | 55.947 | 1.00 | 31.42 | C |
| ATOM | 4 | CG | PRO | A | 221 | 21.395 | 10.756 | 54.830 | 1.00 | 31.70 | C |
| ATOM | 5 | CD | PRO | A | 221 | 20.091 | 11.446 | 55.134 | 1.00 | 31.30 | C |
| ATOM | 6 | C | PRO | A | 221 | 21.630 | 11.069 | 58.108 | 1.00 | 31.12 | C |
| ATOM | 7 | O | PRO | A | 221 | 21.845 | 10.667 | 59.260 | 1.00 | 31.71 | O |
| ATOM | 15 | N | ASN | A | 222 | 22.025 | 12.248 | 57.626 | 1.00 | 30.29 | N |
| ATOM | 16 | CA | ASN | A | 222 | 22.886 | 13.170 | 58.351 | 1.00 | 29.66 | C |
| ATOM | 17 | CB | ASN | A | 222 | 23.483 | 14.136 | 57.322 | 1.00 | 29.96 | C |
| ATOM | 18 | CG | ASN | A | 222 | 24.696 | 14.891 | 57.842 | 1.00 | 31.45 | C |
| ATOM | 19 | OD1 | ASN | A | 222 | 25.860 | 14.636 | 57.242 | 1.00 | 33.52 | O |
| ATOM | 20 | ND2 | ASN | A | 222 | 24.582 | 15.714 | 58.759 | 1.00 | 30.80 | N |
| ATOM | 21 | C | ASN | A | 222 | 22.102 | 13.906 | 59.456 | 1.00 | 28.68 | C |
| ATOM | 22 | O | ASN | A | 222 | 21.881 | 15.118 | 59.381 | 1.00 | 29.00 | O |
| ATOM | 29 | N | VAL | A | 223 | 21.695 | 13.175 | 60.495 | 1.00 | 27.20 | N |
| ATOM | 30 | CA | VAL | A | 223 | 20.790 | 13.709 | 61.523 | 1.00 | 26.08 | C |
| ATOM | 31 | CB | VAL | A | 223 | 20.417 | 12.627 | 62.577 | 1.00 | 25.67 | C |
| ATOM | 32 | CG1 | VAL | A | 223 | 19.438 | 13.173 | 63.626 | 1.00 | 25.61 | C |
| ATOM | 33 | CG2 | VAL | A | 223 | 19.817 | 11.394 | 61.912 | 1.00 | 25.52 | C |
| ATOM | 34 | C | VAL | A | 223 | 21.443 | 14.917 | 62.220 | 1.00 | 25.68 | C |
| ATOM | 35 | O | VAL | A | 223 | 22.627 | 14.842 | 62.558 | 1.00 | 25.95 | O |
| ATOM | 45 | N | PRO | A | 224 | 20.708 | 16.020 | 62.431 | 1.00 | 24.97 | N |
| ATOM | 46 | CA | PRO | A | 224 | 21.288 | 17.199 | 63.098 | 1.00 | 25.02 | C |
| ATOM | 47 | CB | PRO | A | 224 | 20.083 | 18.146 | 63.301 | 1.00 | 24.79 | C |
| ATOM | 48 | CG | PRO | A | 224 | 19.016 | 17.666 | 62.370 | 1.00 | 24.66 | C |
| ATOM | 49 | CD | PRO | A | 224 | 19.291 | 16.231 | 62.074 | 1.00 | 24.65 | C |
| ATOM | 50 | C | PRO | A | 224 | 21.921 | 16.834 | 64.453 | 1.00 | 25.14 | C |
| ATOM | 51 | O | PRO | A | 224 | 21.413 | 15.963 | 65.171 | 1.00 | 25.35 | O |
| ATOM | 59 | N | GLU | A | 225 | 23.010 | 17.498 | 64.805 | 1.00 | 24.91 | N |
| ATOM | 60 | CA | GLU | A | 225 | 23.758 | 17.098 | 65.978 | 1.00 | 25.05 | C |
| ATOM | 61 | CB | GLU | A | 225 | 25.147 | 17.761 | 65.999 | 1.00 | 25.46 | C |
| ATOM | 62 | CG | GLU | A | 225 | 25.251 | 19.065 | 66.756 | 1.00 | 28.01 | C |
| ATOM | 63 | CD | GLU | A | 225 | 26.592 | 19.739 | 66.534 | 1.00 | 32.26 | C |
| ATOM | 64 | OE1 | GLU | A | 225 | 26.950 | 20.003 | 65.348 | 1.00 | 34.55 | O |
| ATOM | 65 | OE2 | GLU | A | 225 | 27.292 | 20.003 | 67.553 | 1.00 | 35.66 | O |
| ATOM | 66 | C | GLU | A | 225 | 22.959 | 17.319 | 67.264 | 1.00 | 24.27 | C |
| ATOM | 67 | O | GLU | A | 225 | 23.053 | 16.518 | 68.186 | 1.00 | 24.47 | O |
| ATOM | 74 | N | LEU | A | 226 | 22.147 | 18.373 | 67.326 | 1.00 | 23.39 | N |
| ATOM | 75 | CA | LEU | A | 226 | 21.289 | 18.584 | 68.490 | 1.00 | 22.56 | C |
| ATOM | 76 | CB | LEU | A | 226 | 20.429 | 19.851 | 68.344 | 1.00 | 22.49 | C |
| ATOM | 77 | CG | LEU | A | 226 | 19.457 | 20.187 | 69.484 | 1.00 | 23.72 | C |
| ATOM | 78 | CD1 | LEU | A | 226 | 20.154 | 20.655 | 70.761 | 1.00 | 23.89 | C |
| ATOM | 79 | CD2 | LEU | A | 226 | 18.461 | 21.248 | 69.038 | 1.00 | 25.85 | C |
| ATOM | 80 | C | LEU | A | 226 | 20.374 | 17.404 | 68.729 | 1.00 | 21.80 | C |
| ATOM | 81 | O | LEU | A | 226 | 20.141 | 17.017 | 69.876 | 1.00 | 20.65 | O |
| ATOM | 93 | N | ILE | A | 227 | 19.821 | 16.860 | 67.653 | 1.00 | 21.23 | N |
| ATOM | 94 | CA | ILE | A | 227 | 18.926 | 15.719 | 67.793 | 1.00 | 21.38 | C |
| ATOM | 95 | CB | ILE | A | 227 | 18.129 | 15.445 | 66.480 | 1.00 | 21.29 | C |
| ATOM | 96 | CG1 | ILE | A | 227 | 17.113 | 16.580 | 66.256 | 1.00 | 21.81 | C |
| ATOM | 97 | CD1 | ILE | A | 227 | 16.198 | 16.411 | 65.050 | 1.00 | 19.55 | C |
| ATOM | 98 | CG2 | ILE | A | 227 | 17.391 | 14.085 | 66.555 | 1.00 | 20.57 | C |
| ATOM | 99 | C | ILE | A | 227 | 19.706 | 14.495 | 68.277 | 1.00 | 21.27 | C |
| ATOM | 100 | O | ILE | A | 227 | 19.210 | 13.721 | 69.093 | 1.00 | 20.95 | O |
| ATOM | 112 | N | LEU | A | 228 | 20.933 | 14.348 | 67.794 | 1.00 | 21.59 | N |
| ATOM | 113 | CA | LEU | A | 228 | 21.781 | 13.237 | 68.211 | 1.00 | 21.96 | C |
| ATOM | 114 | CB | LEU | A | 228 | 23.087 | 13.187 | 67.394 | 1.00 | 21.90 | C |
| ATOM | 115 | CG | LEU | A | 228 | 22.985 | 12.753 | 65.927 | 1.00 | 22.19 | C |
| ATOM | 116 | CD1 | LEU | A | 228 | 24.330 | 12.847 | 65.243 | 1.00 | 22.53 | C |
| ATOM | 117 | CD2 | LEU | A | 228 | 22.460 | 11.333 | 65.797 | 1.00 | 22.94 | C |
| ATOM | 118 | C | LEU | A | 228 | 22.074 | 13.327 | 69.705 | 1.00 | 21.76 | C |
| ATOM | 119 | O | LEU | A | 228 | 21.982 | 12.333 | 70.410 | 1.00 | 21.30 | O |
| ATOM | 131 | N | GLN | A | 229 | 22.385 | 14.530 | 70.179 | 1.00 | 22.09 | N |
| ATOM | 132 | CA | GLN | A | 229 | 22.735 | 14.735 | 71.577 | 1.00 | 22.37 | C |
| ATOM | 133 | CB | GLN | A | 229 | 23.291 | 16.136 | 71.828 | 1.00 | 22.62 | C |
| ATOM | 134 | CG | GLN | A | 229 | 24.781 | 16.290 | 71.481 | 1.00 | 24.53 | C |
| ATOM | 135 | CD | GLN | A | 229 | 25.113 | 17.591 | 70.747 | 1.00 | 26.83 | C |
| ATOM | 136 | OE1 | GLN | A | 229 | 24.389 | 18.585 | 70.866 | 1.00 | 30.47 | O |
| ATOM | 137 | NE2 | GLN | A | 229 | 26.215 | 17.586 | 69.991 | 1.00 | 29.75 | N |
| ATOM | 138 | C | GLN | A | 229 | 21.529 | 14.488 | 72.437 | 1.00 | 22.36 | C |
| ATOM | 139 | O | GLN | A | 229 | 21.664 | 13.927 | 73.509 | 1.00 | 23.16 | O |
| ATOM | 148 | N | LEU | A | 230 | 20.344 | 14.872 | 71.967 | 1.00 | 22.17 | N |
| ATOM | 149 | CA | LEU | A | 230 | 19.117 | 14.611 | 72.713 | 1.00 | 21.90 | C |
| ATOM | 150 | CB | LEU | A | 230 | 17.939 | 15.378 | 72.112 | 1.00 | 22.12 | C |
| ATOM | 151 | CG | LEU | A | 230 | 17.860 | 16.871 | 72.402 | 1.00 | 21.33 | C |
| ATOM | 152 | CD1 | LEU | A | 230 | 16.820 | 17.501 | 71.498 | 1.00 | 21.43 | C |
| ATOM | 153 | CD2 | LEU | A | 230 | 17.530 | 17.115 | 73.837 | 1.00 | 22.18 | C |
| ATOM | 154 | C | LEU | A | 230 | 18.729 | 13.131 | 72.835 | 1.00 | 21.94 | C |
| ATOM | 155 | O | LEU | A | 230 | 18.129 | 12.737 | 73.820 | 1.00 | 22.23 | O |
| ATOM | 167 | N | LEU | A | 231 | 19.030 | 12.320 | 71.832 | 1.00 | 22.27 | N |
| ATOM | 168 | CA | LEU | A | 231 | 18.751 | 10.881 | 71.900 | 1.00 | 22.30 | C |
| ATOM | 169 | CB | LEU | A | 231 | 19.030 | 10.208 | 70.564 | 1.00 | 22.03 | C |
| ATOM | 170 | CG | LEU | A | 231 | 18.053 | 10.602 | 69.467 | 1.00 | 21.89 | C |
| ATOM | 171 | CD1 | LEU | A | 231 | 18.669 | 10.297 | 68.104 | 1.00 | 21.46 | C |
| ATOM | 172 | CD2 | LEU | A | 231 | 16.693 | 9.904 | 69.676 | 1.00 | 20.61 | C |
| ATOM | 173 | C | LEU | A | 231 | 19.615 | 10.219 | 72.934 | 1.00 | 22.38 | C |
| ATOM | 174 | O | LEU | A | 231 | 19.179 | 9.295 | 73.599 | 1.00 | 22.35 | O |
| ATOM | 186 | N | GLN | A | 232 | 20.853 | 10.683 | 73.036 | 1.00 | 22.90 | N |
| ATOM | 187 | CA | GLN | A | 232 | 21.783 | 10.170 | 74.026 | 1.00 | 23.81 | C |
| ATOM | 188 | CB | GLN | A | 232 | 23.197 | 10.691 | 73.746 | 1.00 | 23.79 | C |
| ATOM | 189 | CG | GLN | A | 232 | 23.824 | 10.126 | 72.475 | 1.00 | 25.25 | C |
| ATOM | 190 | CD | GLN | A | 232 | 24.105 | 8.618 | 72.540 | 1.00 | 27.46 | C |
| ATOM | 191 | OE1 | GLN | A | 232 | 23.187 | 7.818 | 72.743 | 1.00 | 30.46 | O |
| ATOM | 192 | NE2 | GLN | A | 232 | 25.366 | 8.231 | 72.346 | 1.00 | 28.30 | N |
| ATOM | 193 | C | GLN | A | 232 | 21.356 | 10.495 | 75.460 | 1.00 | 24.17 | C |
| ATOM | 194 | O | GLN | A | 232 | 21.738 | 9.799 | 76.382 | 1.00 | 23.68 | O |
| ATOM | 203 | N | LEU | A | 233 | 20.565 | 11.551 | 75.641 | 1.00 | 25.36 | N |
| ATOM | 204 | CA | LEU | A | 233 | 20.042 | 11.902 | 76.955 | 1.00 | 26.29 | C |
| ATOM | 205 | CB | LEU | A | 233 | 19.756 | 13.401 | 77.063 | 1.00 | 26.37 | C |
| ATOM | 206 | CG | LEU | A | 233 | 20.852 | 14.374 | 76.621 | 1.00 | 26.99 | C |
| ATOM | 207 | CD1 | LEU | A | 233 | 20.430 | 15.790 | 76.932 | 1.00 | 27.20 | C |
| ATOM | 208 | CD2 | LEU | A | 233 | 22.191 | 14.079 | 77.258 | 1.00 | 28.29 | C |
| ATOM | 209 | C | LEU | A | 233 | 18.765 | 11.158 | 77.264 | 1.00 | 27.41 | C |
| ATOM | 210 | O | LEU | A | 233 | 18.374 | 11.053 | 78.419 | 1.00 | 27.54 | O |
| ATOM | 222 | N | GLU | A | 234 | 18.089 | 10.665 | 76.238 | 1.00 | 28.81 | N |
| ATOM | 223 | CA | GLU | A | 234 | 16.796 | 10.017 | 76.438 | 1.00 | 29.95 | C |
| ATOM | 224 | CB | GLU | A | 234 | 16.184 | 9.618 | 75.082 | 1.00 | 30.25 | C |
| ATOM | 225 | CG | GLU | A | 234 | 14.673 | 9.383 | 75.062 | 1.00 | 31.11 | C |
| ATOM | 226 | CD | GLU | A | 234 | 13.859 | 10.567 | 75.503 | 1.00 | 32.15 | C |
| ATOM | 227 | OE1 | GLU | A | 234 | 14.296 | 11.709 | 75.284 | 1.00 | 34.81 | O |
| ATOM | 228 | OE2 | GLU | A | 234 | 12.775 | 10.359 | 76.088 | 1.00 | 36.52 | O |
| ATOM | 229 | C | GLU | A | 234 | 17.022 | 8.809 | 77.359 | 1.00 | 30.87 | C |
| ATOM | 230 | O | GLU | A | 234 | 17.825 | 7.928 | 77.039 | 1.00 | 31.32 | O |
| ATOM | 237 | N | PRO | A | 235 | 16.366 | 8.791 | 78.522 | 1.00 | 32.07 | N |
| ATOM | 238 | CA | PRO | A | 235 | 16.629 | 7.759 | 79.536 | 1.00 | 33.02 | C |
| ATOM | 239 | CB | PRO | A | 235 | 15.935 | 8.318 | 80.787 | 1.00 | 32.52 | C |
| ATOM | 240 | CG | PRO | A | 235 | 14.817 | 9.079 | 80.254 | 1.00 | 32.38 | C |
| ATOM | 241 | CD | PRO | A | 235 | 15.327 | 9.731 | 78.981 | 1.00 | 32.22 | C |
| ATOM | 242 | C | PRO | A | 235 | 16.027 | 6.403 | 79.187 | 1.00 | 34.42 | C |
| ATOM | 243 | O | PRO | A | 235 | 16.532 | 5.369 | 79.667 | 1.00 | 34.42 | O |
| ATOM | 251 | N | ASP | A | 236 | 14.960 | 6.407 | 78.381 | 1.00 | 35.62 | N |
| ATOM | 252 | CA | ASP | A | 236 | 14.255 | 5.161 | 78.110 | 1.00 | 36.30 | C |
| ATOM | 253 | CB | ASP | A | 236 | 13.056 | 5.358 | 77.167 | 1.00 | 37.21 | C |
| ATOM | 254 | CG | ASP | A | 236 | 11.998 | 4.244 | 77.309 | 1.00 | 38.47 | C |
| ATOM | 255 | OD1 | ASP | A | 236 | 12.046 | 3.477 | 78.310 | 1.00 | 39.87 | O |
| ATOM | 256 | OD2 | ASP | A | 236 | 11.082 | 4.074 | 76.464 | 1.00 | 39.46 | O |
| ATOM | 257 | C | ASP | A | 236 | 15.196 | 4.130 | 77.529 | 1.00 | 36.11 | C |
| ATOM | 258 | O | ASP | A | 236 | 15.715 | 4.285 | 76.411 | 1.00 | 36.58 | O |
| ATOM | 263 | N | GLU | A | 237 | 15.461 | 3.127 | 78.365 | 1.00 | 35.63 | N |
| ATOM | 264 | CA | GLU | A | 237 | 15.920 | 1.815 | 77.958 | 1.00 | 34.76 | C |
| ATOM | 265 | CB | GLU | A | 237 | 17.452 | 1.749 | 78.016 | 1.00 | 34.92 | C |
| ATOM | 266 | CG | GLU | A | 237 | 18.059 | 0.386 | 77.654 | 1.00 | 34.72 | C |
| ATOM | 267 | CD | GLU | A | 237 | 19.339 | 0.480 | 76.821 | 1.00 | 35.23 | C |
| ATOM | 268 | OE1 | GLU | A | 237 | 19.647 | 1.600 | 76.316 | 1.00 | 35.31 | O |
| ATOM | 269 | OE2 | GLU | A | 237 | 20.029 | −0.573 | 76.675 | 1.00 | 33.17 | O |
| ATOM | 270 | C | GLU | A | 237 | 15.233 | 0.802 | 78.920 | 1.00 | 34.49 | C |
| ATOM | 271 | O | GLU | A | 237 | 15.843 | −0.207 | 79.342 | 1.00 | 34.67 | O |
| ATOM | 278 | N | ASP | A | 238 | 13.954 | 1.093 | 79.237 | 1.00 | 33.68 | N |
| ATOM | 279 | CA | ASP | A | 238 | 13.111 | 0.357 | 80.218 | 1.00 | 33.04 | C |
| ATOM | 280 | CB | ASP | A | 238 | 13.355 | −1.173 | 80.194 | 1.00 | 32.75 | C |
| ATOM | 281 | CG | ASP | A | 238 | 12.956 | −1.815 | 78.862 | 1.00 | 32.38 | C |
| ATOM | 282 | OD1 | ASP | A | 238 | 12.070 | −1.262 | 78.185 | 1.00 | 32.82 | O |
| ATOM | 283 | OD2 | ASP | A | 238 | 13.452 | −2.871 | 78.407 | 1.00 | 32.13 | O |
| ATOM | 284 | C | ASP | A | 238 | 13.241 | 0.914 | 81.643 | 1.00 | 32.79 | C |
| ATOM | 285 | O | ASP | A | 238 | 12.569 | 0.466 | 82.582 | 1.00 | 32.38 | O |
| ATOM | 290 | N | GLN | A | 239 | 14.073 | 1.938 | 81.769 | 1.00 | 32.60 | N |
| ATOM | 291 | CA | GLN | A | 239 | 14.556 | 2.396 | 83.057 | 1.00 | 32.49 | C |
| ATOM | 292 | CB | GLN | A | 239 | 15.908 | 3.089 | 82.875 | 1.00 | 32.72 | C |
| ATOM | 293 | CG | GLN | A | 239 | 16.749 | 3.167 | 84.139 | 1.00 | 34.14 | C |
| ATOM | 294 | CD | GLN | A | 239 | 17.662 | 4.394 | 84.162 | 1.00 | 36.23 | C |
| ATOM | 295 | OE1 | GLN | A | 239 | 18.906 | 4.259 | 84.069 | 1.00 | 37.18 | O |
| ATOM | 296 | NE2 | GLN | A | 239 | 17.054 | 5.593 | 84.285 | 1.00 | 36.31 | N |
| ATOM | 297 | C | GLN | A | 239 | 13.563 | 3.346 | 83.691 | 1.00 | 31.96 | C |
| ATOM | 298 | O | GLN | A | 239 | 13.450 | 3.386 | 84.897 | 1.00 | 32.02 | O |
| ATOM | 307 | N | VAL | A | 240 | 12.850 | 4.113 | 82.871 | 1.00 | 31.79 | N |
| ATOM | 308 | CA | VAL | A | 240 | 11.807 | 5.026 | 83.354 | 1.00 | 31.21 | C |
| ATOM | 309 | CB | VAL | A | 240 | 11.184 | 5.791 | 82.162 | 1.00 | 31.13 | C |
| ATOM | 310 | CG1 | VAL | A | 240 | 9.960 | 6.646 | 82.573 | 1.00 | 30.83 | C |
| ATOM | 311 | CG2 | VAL | A | 240 | 12.248 | 6.693 | 81.525 | 1.00 | 31.08 | C |
| ATOM | 312 | C | VAL | A | 240 | 10.759 | 4.230 | 84.165 | 1.00 | 31.32 | C |
| ATOM | 313 | O | VAL | A | 240 | 10.496 | 4.530 | 85.345 | 1.00 | 31.45 | O |
| ATOM | 323 | N | ARG | A | 241 | 10.218 | 3.186 | 83.538 | 1.00 | 30.93 | N |
| ATOM | 324 | CA | ARG | A | 241 | 9.259 | 2.273 | 84.162 | 1.00 | 30.60 | C |
| ATOM | 325 | CB | ARG | A | 241 | 8.899 | 1.183 | 83.159 | 1.00 | 30.71 | C |
| ATOM | 326 | CG | ARG | A | 241 | 7.929 | 0.147 | 83.673 | 1.00 | 31.52 | C |
| ATOM | 327 | CD | ARG | A | 241 | 7.162 | −0.579 | 82.572 | 1.00 | 33.05 | C |
| ATOM | 328 | NE | ARG | A | 241 | 7.949 | −0.690 | 81.339 | 1.00 | 34.64 | N |
| ATOM | 329 | CZ | ARG | A | 241 | 7.511 | −0.408 | 80.105 | 1.00 | 35.34 | C |
| ATOM | 330 | NH1 | ARG | A | 241 | 6.257 | 0.006 | 79.887 | 1.00 | 36.86 | N |
| ATOM | 331 | NH2 | ARG | A | 241 | 8.343 | −0.545 | 79.071 | 1.00 | 35.17 | N |
| ATOM | 332 | C | ARG | A | 241 | 9.740 | 1.613 | 85.457 | 1.00 | 30.38 | C |
| ATOM | 333 | O | ARG | A | 241 | 8.975 | 1.503 | 86.420 | 1.00 | 31.06 | O |
| ATOM | 347 | N | ALA | A | 242 | 10.978 | 1.135 | 85.474 | 1.00 | 29.97 | N |
| ATOM | 348 | CA | ALA | A | 242 | 11.552 | 0.553 | 86.688 | 1.00 | 29.74 | C |
| ATOM | 349 | CB | ALA | A | 242 | 12.966 | 0.046 | 86.410 | 1.00 | 29.59 | C |
| ATOM | 350 | C | ALA | A | 242 | 11.575 | 1.600 | 87.804 | 1.00 | 29.95 | C |
| ATOM | 351 | O | ALA | A | 242 | 11.000 | 1.413 | 88.881 | 1.00 | 29.83 | O |
| ATOM | 357 | N | ARG | A | 243 | 12.224 | 2.720 | 87.508 | 1.00 | 30.42 | N |
| ATOM | 358 | CA | ARG | A | 243 | 12.383 | 3.819 | 88.455 | 1.00 | 30.93 | C |
| ATOM | 359 | CB | ARG | A | 243 | 13.206 | 4.997 | 87.848 | 1.00 | 30.98 | C |
| ATOM | 360 | CG | ARG | A | 243 | 14.530 | 4.597 | 87.092 | 1.00 | 31.63 | C |
| ATOM | 361 | CD | ARG | A | 243 | 15.868 | 5.112 | 87.683 | 1.00 | 32.42 | C |
| ATOM | 362 | NE | ARG | A | 243 | 16.781 | 4.037 | 88.116 | 1.00 | 33.83 | N |
| ATOM | 363 | CZ | ARG | A | 243 | 18.020 | 4.230 | 88.600 | 1.00 | 33.23 | C |
| ATOM | 364 | NH1 | ARG | A | 243 | 18.522 | 5.465 | 88.720 | 1.00 | 33.18 | N |
| ATOM | 365 | NH2 | ARG | A | 243 | 18.758 | 3.184 | 88.974 | 1.00 | 32.26 | N |
| ATOM | 366 | C | ARG | A | 243 | 11.027 | 4.328 | 88.979 | 1.00 | 31.35 | C |
| ATOM | 367 | O | ARG | A | 243 | 10.981 | 4.837 | 90.093 | 1.00 | 32.37 | O |
| ATOM | 381 | N | ILE | A | 244 | 9.928 | 4.190 | 88.224 | 1.00 | 31.35 | N |
| ATOM | 382 | CA | ILE | A | 244 | 8.628 | 4.722 | 88.691 | 1.00 | 31.19 | C |
| ATOM | 383 | CB | ILE | A | 244 | 7.633 | 4.951 | 87.490 | 1.00 | 30.92 | C |
| ATOM | 384 | CG1 | ILE | A | 244 | 8.198 | 6.084 | 86.606 | 1.00 | 30.47 | C |
| ATOM | 385 | CD1 | ILE | A | 244 | 7.202 | 7.094 | 86.014 | 1.00 | 30.15 | C |
| ATOM | 386 | CG2 | ILE | A | 244 | 6.189 | 5.234 | 87.978 | 1.00 | 30.30 | C |
| ATOM | 387 | C | ILE | A | 244 | 8.078 | 3.876 | 89.855 | 1.00 | 31.38 | C |
| ATOM | 388 | O | ILE | A | 244 | 8.013 | 2.650 | 89.760 | 1.00 | 31.07 | O |
| ATOM | 400 | N | LEU | A | 245 | 7.693 | 4.581 | 90.938 | 1.00 | 32.28 | N |
| ATOM | 401 | CA | LEU | A | 245 | 7.757 | 4.086 | 92.358 | 1.00 | 32.82 | C |
| ATOM | 402 | CB | LEU | A | 245 | 8.495 | 5.111 | 93.248 | 1.00 | 32.92 | C |
| ATOM | 403 | CG | LEU | A | 245 | 10.022 | 5.177 | 93.220 | 1.00 | 33.54 | C |
| ATOM | 404 | CD1 | LEU | A | 245 | 10.522 | 6.580 | 93.642 | 1.00 | 33.91 | C |
| ATOM | 405 | CD2 | LEU | A | 245 | 10.653 | 4.071 | 94.098 | 1.00 | 34.68 | C |
| ATOM | 406 | C | LEU | A | 245 | 6.448 | 3.780 | 93.120 | 1.00 | 32.99 | C |
| ATOM | 407 | O | LEU | A | 245 | 6.519 | 3.314 | 94.270 | 1.00 | 33.20 | O |
| ATOM | 419 | N | GLY | A | 246 | 5.276 | 4.085 | 92.556 | 1.00 | 33.22 | N |
| ATOM | 420 | CA | GLY | A | 246 | 4.055 | 3.519 | 93.118 | 1.00 | 33.39 | C |
| ATOM | 421 | C | GLY | A | 246 | 4.295 | 2.012 | 93.198 | 1.00 | 33.55 | C |
| ATOM | 422 | O | GLY | A | 246 | 4.530 | 1.350 | 92.176 | 1.00 | 34.19 | O |
| ATOM | 426 | N | SER | A | 247 | 4.275 | 1.452 | 94.399 | 1.00 | 33.28 | N |
| ATOM | 427 | CA | SER | A | 247 | 4.957 | 0.182 | 94.587 | 1.00 | 33.23 | C |
| ATOM | 428 | CB | SER | A | 247 | 5.817 | 0.245 | 95.869 | 1.00 | 33.55 | C |
| ATOM | 429 | OG | SER | A | 247 | 6.769 | 1.308 | 95.766 | 1.00 | 33.87 | O |
| ATOM | 430 | C | SER | A | 247 | 4.075 | −1.099 | 94.507 | 1.00 | 33.03 | C |
| ATOM | 431 | O | SER | A | 247 | 4.097 | −1.944 | 95.419 | 1.00 | 32.95 | O |
| ATOM | 437 | N | LEU | A | 248 | 3.335 | −1.234 | 93.393 | 1.00 | 32.48 | N |
| ATOM | 438 | CA | LEU | A | 248 | 2.766 | −2.530 | 92.935 | 1.00 | 31.66 | C |
| ATOM | 439 | CB | LEU | A | 248 | 3.829 | −3.361 | 92.163 | 1.00 | 31.38 | C |
| ATOM | 440 | CG | LEU | A | 248 | 4.535 | −2.785 | 90.915 | 1.00 | 29.42 | C |
| ATOM | 441 | CD1 | LEU | A | 248 | 5.668 | −3.701 | 90.410 | 1.00 | 27.71 | C |
| ATOM | 442 | CD2 | LEU | A | 248 | 3.535 | −2.534 | 89.795 | 1.00 | 28.74 | C |
| ATOM | 443 | C | LEU | A | 248 | 2.168 | −3.390 | 94.057 | 1.00 | 31.65 | C |
| ATOM | 444 | O | LEU | A | 248 | 2.636 | −4.502 | 94.334 | 1.00 | 31.42 | O |
| ATOM | 456 | N | PRO | A | 256 | −13.963 | −10.661 | 86.340 | 1.00 | 29.31 | N |
| ATOM | 457 | CA | PRO | A | 256 | −14.062 | −9.960 | 87.630 | 1.00 | 29.13 | C |
| ATOM | 458 | CB | PRO | A | 256 | −13.772 | −11.084 | 88.652 | 1.00 | 29.36 | C |
| ATOM | 459 | CG | PRO | A | 256 | −14.306 | −12.402 | 87.954 | 1.00 | 29.16 | C |
| ATOM | 460 | CD | PRO | A | 256 | −14.415 | −12.067 | 86.451 | 1.00 | 29.39 | C |
| ATOM | 461 | C | PRO | A | 256 | −13.064 | −8.811 | 87.770 | 1.00 | 29.05 | C |
| ATOM | 462 | O | PRO | A | 256 | −12.108 | −8.943 | 88.543 | 1.00 | 29.49 | O |
| ATOM | 470 | N | ASP | A | 257 | −13.281 | −7.706 | 87.049 | 1.00 | 28.58 | N |
| ATOM | 471 | CA | ASP | A | 257 | −12.420 | −6.522 | 87.195 | 1.00 | 28.52 | C |
| ATOM | 472 | CB | ASP | A | 257 | −11.670 | −6.216 | 85.891 | 1.00 | 28.59 | C |
| ATOM | 473 | CG | ASP | A | 257 | −10.181 | −6.549 | 85.993 | 1.00 | 29.04 | C |
| ATOM | 474 | OD1 | ASP | A | 257 | −9.844 | −7.668 | 86.472 | 1.00 | 29.76 | O |
| ATOM | 475 | OD2 | ASP | A | 257 | −9.284 | −5.752 | 85.636 | 1.00 | 29.42 | O |
| ATOM | 476 | C | ASP | A | 257 | −13.145 | −5.283 | 87.763 | 1.00 | 28.19 | C |
| ATOM | 477 | O | ASP | A | 257 | −14.358 | −5.306 | 87.956 | 1.00 | 28.40 | O |
| ATOM | 482 | N | GLN | A | 258 | −12.384 | −4.213 | 88.022 | 1.00 | 27.26 | N |
| ATOM | 483 | CA | GLN | A | 258 | −12.690 | −3.303 | 89.135 | 1.00 | 26.55 | C |
| ATOM | 484 | CB | GLN | A | 258 | −11.721 | −3.555 | 90.322 | 1.00 | 27.13 | C |
| ATOM | 485 | CG | GLN | A | 258 | −10.625 | −4.625 | 90.132 | 1.00 | 29.11 | C |
| ATOM | 486 | CD | GLN | A | 258 | −9.278 | −4.059 | 89.669 | 1.00 | 31.47 | C |
| ATOM | 487 | OE1 | GLN | A | 258 | −9.160 | −2.877 | 89.309 | 1.00 | 34.18 | O |
| ATOM | 488 | NE2 | GLN | A | 258 | −8.256 | −4.915 | 89.677 | 1.00 | 33.92 | N |
| ATOM | 489 | C | GLN | A | 258 | −12.675 | −1.821 | 88.784 | 1.00 | 25.03 | C |
| ATOM | 490 | O | GLN | A | 258 | −12.436 | −1.460 | 87.643 | 1.00 | 24.89 | O |
| ATOM | 499 | N | PRO | A | 259 | −12.964 | −0.958 | 89.759 | 1.00 | 23.54 | N |
| ATOM | 500 | CA | PRO | A | 259 | −12.810 | 0.489 | 89.557 | 1.00 | 22.95 | C |
| ATOM | 501 | CB | PRO | A | 259 | −13.391 | 1.081 | 90.854 | 1.00 | 22.93 | C |
| ATOM | 502 | CG | PRO | A | 259 | −14.229 | −0.017 | 91.459 | 1.00 | 22.46 | C |
| ATOM | 503 | CD | PRO | A | 259 | −13.508 | −1.260 | 91.102 | 1.00 | 23.17 | C |
| ATOM | 504 | C | PRO | A | 259 | −11.352 | 0.956 | 89.334 | 1.00 | 22.51 | C |
| ATOM | 505 | O | PRO | A | 259 | −10.404 | 0.315 | 89.775 | 1.00 | 21.72 | O |
| ATOM | 513 | N | ALA | A | 260 | −11.190 | 2.077 | 88.635 | 1.00 | 22.85 | N |
| ATOM | 514 | CA | ALA | A | 260 | −9.867 | 2.686 | 88.417 | 1.00 | 22.95 | C |
| ATOM | 515 | CB | ALA | A | 260 | −9.980 | 3.975 | 87.616 | 1.00 | 22.96 | C |
| ATOM | 516 | C | ALA | A | 260 | −9.180 | 2.961 | 89.747 | 1.00 | 23.03 | C |
| ATOM | 517 | O | ALA | A | 260 | −9.820 | 3.398 | 90.708 | 1.00 | 23.27 | O |
| ATOM | 523 | N | ALA | A | 261 | −7.883 | 2.682 | 89.797 | 1.00 | 22.89 | N |
| ATOM | 524 | CA | ALA | A | 261 | −7.069 | 2.951 | 90.980 | 1.00 | 22.55 | C |
| ATOM | 525 | CB | ALA | A | 261 | −5.858 | 2.019 | 90.985 | 1.00 | 22.54 | C |
| ATOM | 526 | C | ALA | A | 261 | −6.628 | 4.422 | 91.000 | 1.00 | 22.31 | C |
| ATOM | 527 | O | ALA | A | 261 | −5.475 | 4.724 | 90.727 | 1.00 | 22.45 | O |
| ATOM | 533 | N | PHE | A | 262 | −7.553 | 5.323 | 91.335 | 1.00 | 21.98 | N |
| ATOM | 534 | CA | PHE | A | 262 | −7.343 | 6.772 | 91.242 | 1.00 | 21.66 | C |
| ATOM | 535 | CB | PHE | A | 262 | −8.564 | 7.536 | 91.792 | 1.00 | 21.62 | C |
| ATOM | 536 | CG | PHE | A | 262 | −8.531 | 9.028 | 91.514 | 1.00 | 22.25 | C |
| ATOM | 537 | CD1 | PHE | A | 262 | −8.998 | 9.538 | 90.316 | 1.00 | 24.99 | C |
| ATOM | 538 | CE1 | PHE | A | 262 | −8.956 | 10.927 | 90.054 | 1.00 | 24.78 | C |
| ATOM | 539 | CZ | PHE | A | 262 | −8.454 | 11.796 | 90.998 | 1.00 | 23.51 | C |
| ATOM | 540 | CE2 | PHE | A | 262 | −7.985 | 11.308 | 92.182 | 1.00 | 22.70 | C |
| ATOM | 541 | CD2 | PHE | A | 262 | −8.030 | 9.922 | 92.447 | 1.00 | 23.43 | C |
| ATOM | 542 | C | PHE | A | 262 | −6.059 | 7.289 | 91.905 | 1.00 | 21.42 | C |
| ATOM | 543 | O | PHE | A | 262 | −5.257 | 7.939 | 91.240 | 1.00 | 22.01 | O |
| ATOM | 553 | N | GLY | A | 263 | −5.874 | 7.031 | 93.196 | 1.00 | 21.28 | N |
| ATOM | 554 | CA | GLY | A | 263 | −4.713 | 7.536 | 93.919 | 1.00 | 21.27 | C |
| ATOM | 555 | C | GLY | A | 263 | −3.387 | 7.088 | 93.320 | 1.00 | 21.23 | C |
| ATOM | 556 | O | GLY | A | 263 | −2.401 | 7.809 | 93.311 | 1.00 | 21.23 | O |
| ATOM | 560 | N | LEU | A | 264 | −3.377 | 5.869 | 92.819 | 1.00 | 21.13 | N |
| ATOM | 561 | CA | LEU | A | 264 | −2.192 | 5.261 | 92.264 | 1.00 | 21.46 | C |
| ATOM | 562 | CB | LEU | A | 264 | −2.413 | 3.743 | 92.184 | 1.00 | 21.82 | C |
| ATOM | 563 | CG | LEU | A | 264 | −1.217 | 2.831 | 92.344 | 1.00 | 23.71 | C |
| ATOM | 564 | CD1 | LEU | A | 264 | −0.232 | 3.001 | 91.177 | 1.00 | 25.37 | C |
| ATOM | 565 | CD2 | LEU | A | 264 | −0.551 | 3.055 | 93.723 | 1.00 | 26.08 | C |
| ATOM | 566 | C | LEU | A | 264 | −1.916 | 5.853 | 90.895 | 1.00 | 20.85 | C |
| ATOM | 567 | O | LEU | A | 264 | −0.783 | 6.068 | 90.527 | 1.00 | 20.79 | O |
| ATOM | 579 | N | LEU | A | 265 | −2.972 | 6.134 | 90.149 | 1.00 | 20.83 | N |
| ATOM | 580 | CA | LEU | A | 265 | −2.842 | 6.800 | 88.862 | 1.00 | 20.82 | C |
| ATOM | 581 | CB | LEU | A | 265 | −4.197 | 6.840 | 88.126 | 1.00 | 20.71 | C |
| ATOM | 582 | CG | LEU | A | 265 | −4.706 | 5.460 | 87.649 | 1.00 | 20.97 | C |
| ATOM | 583 | CD1 | LEU | A | 265 | −6.140 | 5.511 | 87.155 | 1.00 | 19.98 | C |
| ATOM | 584 | CD2 | LEU | A | 265 | −3.787 | 4.908 | 86.583 | 1.00 | 20.44 | C |
| ATOM | 585 | C | LEU | A | 265 | −2.278 | 8.190 | 89.048 | 1.00 | 20.79 | C |
| ATOM | 586 | O | LEU | A | 265 | −1.487 | 8.646 | 88.235 | 1.00 | 20.83 | O |
| ATOM | 598 | N | CYS | A | 266 | −2.687 | 8.848 | 90.128 | 1.00 | 21.00 | N |
| ATOM | 599 | CA | CYS | A | 266 | −2.173 | 10.165 | 90.495 | 1.00 | 21.27 | C |
| ATOM | 600 | CB | CYS | A | 266 | −2.921 | 10.730 | 91.704 | 1.00 | 20.77 | C |
| ATOM | 601 | SG | CYS | A | 266 | −4.608 | 11.260 | 91.325 | 1.00 | 22.47 | S |
| ATOM | 602 | C | CYS | A | 266 | −0.697 | 10.084 | 90.810 | 1.00 | 21.11 | C |
| ATOM | 603 | O | CYS | A | 266 | 0.076 | 10.860 | 90.304 | 1.00 | 20.94 | O |
| ATOM | 609 | N | ARG | A | 267 | −0.340 | 9.129 | 91.649 | 1.00 | 21.40 | N |
| ATOM | 610 | CA | ARG | A | 267 | 1.040 | 8.855 | 92.026 | 1.00 | 22.25 | C |
| ATOM | 611 | CB | ARG | A | 267 | 1.096 | 7.599 | 92.907 | 1.00 | 22.94 | C |
| ATOM | 612 | CG | ARG | A | 267 | 1.828 | 7.725 | 94.225 | 1.00 | 25.90 | C |
| ATOM | 613 | CD | ARG | A | 267 | 1.088 | 7.037 | 95.385 | 1.00 | 29.27 | C |
| ATOM | 614 | NE | ARG | A | 267 | −0.120 | 7.794 | 95.747 | 1.00 | 32.39 | N |
| ATOM | 615 | CZ | ARG | A | 267 | −1.275 | 7.279 | 96.222 | 1.00 | 34.13 | C |
| ATOM | 616 | NH1 | ARG | A | 267 | −1.442 | 5.960 | 96.419 | 1.00 | 34.21 | N |
| ATOM | 617 | NH2 | ARG | A | 267 | −2.285 | 8.106 | 96.512 | 1.00 | 34.21 | N |
| ATOM | 618 | C | ARG | A | 267 | 1.918 | 8.622 | 90.807 | 1.00 | 21.47 | C |
| ATOM | 619 | O | ARG | A | 267 | 3.024 | 9.113 | 90.734 | 1.00 | 21.61 | O |
| ATOM | 633 | N | MET | A | 268 | 1.401 | 7.853 | 89.863 | 1.00 | 21.10 | N |
| ATOM | 634 | CA | MET | A | 268 | 2.111 | 7.499 | 88.645 | 1.00 | 20.53 | C |
| ATOM | 635 | CB | MET | A | 268 | 1.264 | 6.522 | 87.832 | 1.00 | 20.55 | C |
| ATOM | 636 | CG | MET | A | 268 | 1.774 | 6.208 | 86.454 | 1.00 | 20.15 | C |
| ATOM | 637 | SD | MET | A | 268 | 0.536 | 5.334 | 85.500 | 1.00 | 20.07 | S |
| ATOM | 638 | CE | MET | A | 268 | −0.412 | 6.684 | 84.990 | 1.00 | 20.41 | C |
| ATOM | 639 | C | MET | A | 268 | 2.416 | 8.717 | 87.825 | 1.00 | 19.68 | C |
| ATOM | 640 | O | MET | A | 268 | 3.487 | 8.823 | 87.273 | 1.00 | 19.96 | O |
| ATOM | 650 | N | ALA | A | 269 | 1.458 | 9.630 | 87.749 | 1.00 | 19.50 | N |
| ATOM | 651 | CA | ALA | A | 269 | 1.600 | 10.873 | 86.989 | 1.00 | 19.16 | C |
| ATOM | 652 | CB | ALA | A | 269 | 0.243 | 11.562 | 86.815 | 1.00 | 18.96 | C |
| ATOM | 653 | C | ALA | A | 269 | 2.602 | 11.825 | 87.635 | 1.00 | 18.77 | C |
| ATOM | 654 | O | ALA | A | 269 | 3.337 | 12.530 | 86.935 | 1.00 | 17.62 | O |
| ATOM | 660 | N | ASP | A | 270 | 2.630 | 11.806 | 88.965 | 1.00 | 18.70 | N |
| ATOM | 661 | CA | ASP | A | 270 | 3.518 | 12.642 | 89.759 | 1.00 | 19.28 | C |
| ATOM | 662 | CB | ASP | A | 270 | 3.197 | 12.497 | 91.256 | 1.00 | 19.29 | C |
| ATOM | 663 | CG | ASP | A | 270 | 2.056 | 13.400 | 91.721 | 1.00 | 20.26 | C |
| ATOM | 664 | OD1 | ASP | A | 270 | 1.536 | 14.198 | 90.895 | 1.00 | 20.42 | O |
| ATOM | 665 | OD2 | ASP | A | 270 | 1.623 | 13.368 | 92.912 | 1.00 | 19.70 | O |
| ATOM | 666 | C | ASP | A | 270 | 4.949 | 12.209 | 89.543 | 1.00 | 19.62 | C |
| ATOM | 667 | O | ASP | A | 270 | 5.840 | 13.037 | 89.447 | 1.00 | 19.68 | O |
| ATOM | 672 | N | GLN | A | 271 | 5.158 | 10.899 | 89.493 | 1.00 | 19.39 | N |
| ATOM | 673 | CA | GLN | A | 271 | 6.478 | 10.337 | 89.297 | 1.00 | 19.83 | C |
| ATOM | 674 | CB | GLN | A | 271 | 6.469 | 8.827 | 89.587 | 1.00 | 20.42 | C |
| ATOM | 675 | CG | GLN | A | 271 | 6.333 | 8.468 | 91.036 | 1.00 | 21.69 | C |
| ATOM | 676 | CD | GLN | A | 271 | 7.444 | 9.071 | 91.878 | 1.00 | 24.42 | C |
| ATOM | 677 | OE1 | GLN | A | 271 | 8.633 | 8.932 | 91.549 | 1.00 | 26.23 | O |
| ATOM | 678 | NE2 | GLN | A | 271 | 7.066 | 9.756 | 92.958 | 1.00 | 25.76 | N |
| ATOM | 679 | C | GLN | A | 271 | 6.967 | 10.572 | 87.887 | 1.00 | 19.13 | C |
| ATOM | 680 | O | GLN | A | 271 | 8.142 | 10.684 | 87.659 | 1.00 | 18.69 | O |
| ATOM | 689 | N | THR | A | 272 | 6.045 | 10.609 | 86.943 | 1.00 | 19.15 | N |
| ATOM | 690 | CA | THR | A | 272 | 6.358 | 10.933 | 85.568 | 1.00 | 19.05 | C |
| ATOM | 691 | CB | THR | A | 272 | 5.160 | 10.696 | 84.651 | 1.00 | 18.45 | C |
| ATOM | 692 | OG1 | THR | A | 272 | 4.728 | 9.345 | 84.760 | 1.00 | 19.57 | O |
| ATOM | 693 | CG2 | THR | A | 272 | 5.549 | 10.821 | 83.220 | 1.00 | 19.11 | C |
| ATOM | 694 | C | THR | A | 272 | 6.769 | 12.380 | 85.501 | 1.00 | 19.13 | C |
| ATOM | 695 | O | THR | A | 272 | 7.673 | 12.731 | 84.751 | 1.00 | 19.69 | O |
| ATOM | 703 | N | PHE | A | 273 | 6.137 | 13.231 | 86.293 | 1.00 | 18.74 | N |
| ATOM | 704 | CA | PHE | A | 273 | 6.549 | 14.612 | 86.273 | 1.00 | 19.24 | C |
| ATOM | 705 | CB | PHE | A | 273 | 5.619 | 15.477 | 87.115 | 1.00 | 19.99 | C |
| ATOM | 706 | CG | PHE | A | 273 | 6.081 | 16.899 | 87.248 | 1.00 | 20.55 | C |
| ATOM | 707 | CD1 | PHE | A | 273 | 6.379 | 17.430 | 88.487 | 1.00 | 21.35 | C |
| ATOM | 708 | CE1 | PHE | A | 273 | 6.819 | 18.731 | 88.589 | 1.00 | 22.29 | C |
| ATOM | 709 | CZ | PHE | A | 273 | 6.973 | 19.506 | 87.470 | 1.00 | 21.03 | C |
| ATOM | 710 | CE2 | PHE | A | 273 | 6.702 | 18.987 | 86.242 | 1.00 | 23.16 | C |
| ATOM | 711 | CD2 | PHE | A | 273 | 6.256 | 17.688 | 86.125 | 1.00 | 22.85 | C |
| ATOM | 712 | C | PHE | A | 273 | 7.973 | 14.695 | 86.771 | 1.00 | 18.84 | C |
| ATOM | 713 | O | PHE | A | 273 | 8.797 | 15.384 | 86.203 | 1.00 | 18.95 | O |
| ATOM | 723 | N | ILE | A | 274 | 8.255 | 13.965 | 87.832 | 1.00 | 19.04 | N |
| ATOM | 724 | CA | ILE | A | 274 | 9.578 | 13.922 | 88.407 | 1.00 | 19.10 | C |
| ATOM | 725 | CB | ILE | A | 274 | 9.622 | 12.888 | 89.547 | 1.00 | 19.68 | C |
| ATOM | 726 | CG1 | ILE | A | 274 | 8.636 | 13.277 | 90.660 | 1.00 | 20.59 | C |
| ATOM | 727 | CD1 | ILE | A | 274 | 9.213 | 13.614 | 92.013 | 1.00 | 21.59 | C |
| ATOM | 728 | CG2 | ILE | A | 274 | 11.075 | 12.671 | 90.017 | 1.00 | 19.28 | C |
| ATOM | 729 | C | ILE | A | 274 | 10.622 | 13.570 | 87.375 | 1.00 | 18.68 | C |
| ATOM | 730 | O | ILE | A | 274 | 11.663 | 14.214 | 87.301 | 1.00 | 17.59 | O |
| ATOM | 742 | N | SER | A | 275 | 10.329 | 12.546 | 86.583 | 1.00 | 18.85 | N |
| ATOM | 743 | CA | SER | A | 275 | 11.261 | 12.053 | 85.586 | 1.00 | 19.69 | C |
| ATOM | 744 | CB | SER | A | 275 | 10.813 | 10.674 | 85.087 | 1.00 | 19.86 | C |
| ATOM | 745 | OG | SER | A | 275 | 9.782 | 10.787 | 84.126 | 1.00 | 22.52 | O |
| ATOM | 746 | C | SER | A | 275 | 11.468 | 13.038 | 84.426 | 1.00 | 19.56 | C |
| ATOM | 747 | O | SER | A | 275 | 12.525 | 13.097 | 83.822 | 1.00 | 19.81 | O |
| ATOM | 753 | N | ILE | A | 276 | 10.447 | 13.829 | 84.136 | 1.00 | 20.02 | N |
| ATOM | 754 | CA | ILE | A | 276 | 10.526 | 14.877 | 83.129 | 1.00 | 19.85 | C |
| ATOM | 755 | CB | ILE | A | 276 | 9.094 | 15.357 | 82.795 | 1.00 | 19.90 | C |
| ATOM | 756 | CG1 | ILE | A | 276 | 8.375 | 14.317 | 81.945 | 1.00 | 19.96 | C |
| ATOM | 757 | CD1 | ILE | A | 276 | 6.916 | 14.616 | 81.763 | 1.00 | 19.95 | C |
| ATOM | 758 | CG2 | ILE | A | 276 | 9.096 | 16.663 | 82.073 | 1.00 | 20.64 | C |
| ATOM | 759 | C | ILE | A | 276 | 11.433 | 16.037 | 83.590 | 1.00 | 19.46 | C |
| ATOM | 760 | O | ILE | A | 276 | 12.130 | 16.622 | 82.784 | 1.00 | 19.34 | O |
| ATOM | 772 | N | VAL | A | 277 | 11.406 | 16.371 | 84.877 | 1.00 | 19.28 | N |
| ATOM | 773 | CA | VAL | A | 277 | 12.287 | 17.398 | 85.430 | 1.00 | 19.33 | C |
| ATOM | 774 | CB | VAL | A | 277 | 11.848 | 17.834 | 86.852 | 1.00 | 19.58 | C |
| ATOM | 775 | CG1 | VAL | A | 277 | 12.831 | 18.822 | 87.471 | 1.00 | 19.89 | C |
| ATOM | 776 | CG2 | VAL | A | 277 | 10.486 | 18.432 | 86.813 | 1.00 | 20.27 | C |
| ATOM | 777 | C | VAL | A | 277 | 13.726 | 16.899 | 85.472 | 1.00 | 18.91 | C |
| ATOM | 778 | O | VAL | A | 277 | 14.635 | 17.663 | 85.221 | 1.00 | 18.14 | O |
| ATOM | 788 | N | ASP | A | 278 | 13.906 | 15.615 | 85.780 | 1.00 | 19.35 | N |
| ATOM | 789 | CA | ASP | A | 278 | 15.200 | 14.934 | 85.684 | 1.00 | 20.07 | C |
| ATOM | 790 | CB | ASP | A | 278 | 15.064 | 13.456 | 86.088 | 1.00 | 20.68 | C |
| ATOM | 791 | CG | ASP | A | 278 | 15.143 | 13.241 | 87.600 | 1.00 | 24.73 | C |
| ATOM | 792 | OD1 | ASP | A | 278 | 15.755 | 14.103 | 88.288 | 1.00 | 30.88 | O |
| ATOM | 793 | OD2 | ASP | A | 278 | 14.654 | 12.229 | 88.202 | 1.00 | 28.68 | O |
| ATOM | 794 | C | ASP | A | 278 | 15.776 | 15.024 | 84.263 | 1.00 | 19.75 | C |
| ATOM | 795 | O | ASP | A | 278 | 16.946 | 15.313 | 84.073 | 1.00 | 19.22 | O |
| ATOM | 800 | N | TRP | A | 279 | 14.919 | 14.811 | 83.279 | 1.00 | 19.61 | N |
| ATOM | 801 | CA | TRP | A | 279 | 15.292 | 14.915 | 81.897 | 1.00 | 19.84 | C |
| ATOM | 802 | CB | TRP | A | 279 | 14.113 | 14.521 | 81.008 | 1.00 | 19.87 | C |
| ATOM | 803 | CG | TRP | A | 279 | 14.319 | 14.966 | 79.617 | 1.00 | 20.67 | C |
| ATOM | 804 | CD1 | TRP | A | 279 | 15.162 | 14.405 | 78.700 | 1.00 | 20.09 | C |
| ATOM | 805 | NE1 | TRP | A | 279 | 15.115 | 15.116 | 77.528 | 1.00 | 21.43 | N |
| ATOM | 806 | CE2 | TRP | A | 279 | 14.228 | 16.152 | 77.660 | 1.00 | 22.16 | C |
| ATOM | 807 | CD2 | TRP | A | 279 | 13.718 | 16.099 | 78.973 | 1.00 | 22.49 | C |
| ATOM | 808 | CE3 | TRP | A | 279 | 12.778 | 17.064 | 79.365 | 1.00 | 22.52 | C |
| ATOM | 809 | CZ3 | TRP | A | 279 | 12.396 | 18.037 | 78.454 | 1.00 | 22.49 | C |
| ATOM | 810 | CH2 | TRP | A | 279 | 12.930 | 18.068 | 77.164 | 1.00 | 23.82 | C |
| ATOM | 811 | CZ2 | TRP | A | 279 | 13.848 | 17.135 | 76.745 | 1.00 | 23.11 | C |
| ATOM | 812 | C | TRP | A | 279 | 15.740 | 16.327 | 81.556 | 1.00 | 20.28 | C |
| ATOM | 813 | O | TRP | A | 279 | 16.804 | 16.513 | 80.981 | 1.00 | 19.67 | O |
| ATOM | 824 | N | ALA | A | 280 | 14.921 | 17.312 | 81.929 | 1.00 | 20.87 | N |
| ATOM | 825 | CA | ALA | A | 280 | 15.156 | 18.699 | 81.570 | 1.00 | 21.43 | C |
| ATOM | 826 | CB | ALA | A | 280 | 13.983 | 19.575 | 81.988 | 1.00 | 21.77 | C |
| ATOM | 827 | C | ALA | A | 280 | 16.427 | 19.209 | 82.200 | 1.00 | 22.16 | C |
| ATOM | 828 | O | ALA | A | 280 | 17.153 | 19.977 | 81.572 | 1.00 | 22.26 | O |
| ATOM | 834 | N | ARG | A | 281 | 16.720 | 18.789 | 83.427 | 1.00 | 22.71 | N |
| ATOM | 835 | CA | ARG | A | 281 | 17.922 | 19.300 | 84.081 | 1.00 | 23.53 | C |
| ATOM | 836 | CB | ARG | A | 281 | 17.836 | 19.216 | 85.606 | 1.00 | 23.92 | C |
| ATOM | 837 | CG | ARG | A | 281 | 18.104 | 17.884 | 86.239 | 1.00 | 25.66 | C |
| ATOM | 838 | CD | ARG | A | 281 | 17.866 | 17.909 | 87.747 | 1.00 | 26.18 | C |
| ATOM | 839 | NE | ARG | A | 281 | 18.914 | 17.205 | 88.486 | 1.00 | 28.32 | N |
| ATOM | 840 | CZ | ARG | A | 281 | 18.938 | 17.057 | 89.815 | 1.00 | 29.33 | C |
| ATOM | 841 | NH1 | ARG | A | 281 | 17.964 | 17.557 | 90.574 | 1.00 | 28.91 | N |
| ATOM | 842 | NH2 | ARG | A | 281 | 19.941 | 16.393 | 90.391 | 1.00 | 29.91 | N |
| ATOM | 843 | C | ARG | A | 281 | 19.226 | 18.727 | 83.502 | 1.00 | 23.49 | C |
| ATOM | 844 | O | ARG | A | 281 | 20.281 | 19.333 | 83.647 | 1.00 | 23.19 | O |
| ATOM | 858 | N | ARG | A | 282 | 19.124 | 17.608 | 82.791 | 1.00 | 23.61 | N |
| ATOM | 859 | CA | ARG | A | 282 | 20.238 | 17.063 | 82.025 | 1.00 | 23.57 | C |
| ATOM | 860 | CB | ARG | A | 282 | 20.114 | 15.537 | 81.916 | 1.00 | 23.90 | C |
| ATOM | 861 | CG | ARG | A | 282 | 20.216 | 14.774 | 83.240 | 1.00 | 24.46 | C |
| ATOM | 862 | CD | ARG | A | 282 | 20.272 | 13.235 | 83.069 | 1.00 | 26.55 | C |
| ATOM | 863 | NE | ARG | A | 282 | 18.943 | 12.616 | 83.098 | 1.00 | 29.91 | N |
| ATOM | 864 | CZ | ARG | A | 282 | 18.131 | 12.434 | 82.034 | 1.00 | 31.88 | C |
| ATOM | 865 | NH1 | ARG | A | 282 | 18.504 | 12.815 | 80.815 | 1.00 | 33.77 | N |
| ATOM | 866 | NH2 | ARG | A | 282 | 16.925 | 11.867 | 82.187 | 1.00 | 30.91 | N |
| ATOM | 867 | C | ARG | A | 282 | 20.375 | 17.655 | 80.608 | 1.00 | 22.91 | C |
| ATOM | 868 | O | ARG | A | 282 | 21.405 | 17.460 | 79.995 | 1.00 | 23.29 | O |
| ATOM | 882 | N | CYS | A | 283 | 19.357 | 18.351 | 80.094 | 1.00 | 22.32 | N |
| ATOM | 883 | CA | CYS | A | 283 | 19.380 | 18.877 | 78.719 | 1.00 | 22.08 | C |
| ATOM | 884 | CB | CYS | A | 283 | 18.069 | 19.510 | 78.299 | 1.00 | 21.67 | C |
| ATOM | 885 | SG | CYS | A | 283 | 16.828 | 18.355 | 77.775 | 1.00 | 22.32 | S |
| ATOM | 886 | C | CYS | A | 283 | 20.417 | 19.946 | 78.524 | 1.00 | 22.31 | C |
| ATOM | 887 | O | CYS | A | 283 | 20.618 | 20.777 | 79.406 | 1.00 | 22.76 | O |
| ATOM | 893 | N | MET | A | 284 | 21.040 | 19.931 | 77.343 | 1.00 | 21.98 | N |
| ATOM | 894 | CA | MET | A | 284 | 21.910 | 20.999 | 76.907 | 1.00 | 21.87 | C |
| ATOM | 895 | CB | MET | A | 284 | 22.466 | 20.734 | 75.492 | 1.00 | 22.32 | C |
| ATOM | 896 | CG | MET | A | 284 | 21.449 | 20.730 | 74.308 | 1.00 | 24.04 | C |
| ATOM | 897 | SD | MET | A | 284 | 20.225 | 19.312 | 74.237 | 1.00 | 25.84 | S |
| ATOM | 898 | CE | MET | A | 284 | 21.401 | 18.025 | 73.776 | 1.00 | 22.40 | C |
| ATOM | 899 | C | MET | A | 284 | 21.143 | 22.302 | 76.972 | 1.00 | 21.33 | C |
| ATOM | 900 | O | MET | A | 284 | 19.927 | 22.320 | 76.853 | 1.00 | 20.48 | O |
| ATOM | 910 | N | VAL | A | 285 | 21.892 | 23.380 | 77.176 | 1.00 | 21.35 | N |
| ATOM | 911 | CA | VAL | A | 285 | 21.386 | 24.750 | 77.325 | 1.00 | 20.78 | C |
| ATOM | 912 | CB | VAL | A | 285 | 20.481 | 25.197 | 76.165 | 1.00 | 20.95 | C |
| ATOM | 913 | CG1 | VAL | A | 285 | 20.141 | 26.687 | 76.313 | 1.00 | 21.40 | C |
| ATOM | 914 | CG2 | VAL | A | 285 | 21.163 | 24.934 | 74.806 | 1.00 | 20.78 | C |
| ATOM | 915 | C | VAL | A | 285 | 20.723 | 24.988 | 78.671 | 1.00 | 20.34 | C |
| ATOM | 916 | O | VAL | A | 285 | 21.141 | 25.883 | 79.400 | 1.00 | 19.80 | O |
| ATOM | 926 | N | PHE | A | 286 | 19.694 | 24.187 | 78.971 | 1.00 | 20.24 | N |
| ATOM | 927 | CA | PHE | A | 286 | 18.949 | 24.220 | 80.217 | 1.00 | 19.94 | C |
| ATOM | 928 | CB | PHE | A | 286 | 17.871 | 23.121 | 80.226 | 1.00 | 19.80 | C |
| ATOM | 929 | CG | PHE | A | 286 | 16.852 | 23.293 | 81.326 | 1.00 | 20.12 | C |
| ATOM | 930 | CD1 | PHE | A | 286 | 15.677 | 24.026 | 81.105 | 1.00 | 19.76 | C |
| ATOM | 931 | CE1 | PHE | A | 286 | 14.765 | 24.199 | 82.109 | 1.00 | 18.63 | C |
| ATOM | 932 | CZ | PHE | A | 286 | 14.994 | 23.663 | 83.355 | 1.00 | 19.66 | C |
| ATOM | 933 | CE2 | PHE | A | 286 | 16.159 | 22.953 | 83.607 | 1.00 | 19.43 | C |
| ATOM | 934 | CD2 | PHE | A | 286 | 17.081 | 22.776 | 82.598 | 1.00 | 19.92 | C |
| ATOM | 935 | C | PHE | A | 286 | 19.846 | 24.070 | 81.451 | 1.00 | 20.29 | C |
| ATOM | 936 | O | PHE | A | 286 | 19.726 | 24.822 | 82.421 | 1.00 | 20.06 | O |
| ATOM | 946 | N | LYS | A | 287 | 20.735 | 23.092 | 81.415 | 1.00 | 20.09 | N |
| ATOM | 947 | CA | LYS | A | 287 | 21.634 | 22.856 | 82.519 | 1.00 | 20.34 | C |
| ATOM | 948 | CB | LYS | A | 287 | 22.356 | 21.539 | 82.297 | 1.00 | 20.86 | C |
| ATOM | 949 | CG | LYS | A | 287 | 23.286 | 21.528 | 81.099 | 1.00 | 21.59 | C |
| ATOM | 950 | CD | LYS | A | 287 | 23.758 | 20.111 | 80.846 | 1.00 | 23.05 | C |
| ATOM | 951 | CE | LYS | A | 287 | 24.755 | 20.044 | 79.733 | 1.00 | 23.01 | C |
| ATOM | 952 | NZ | LYS | A | 287 | 25.659 | 18.915 | 79.951 | 1.00 | 23.69 | N |
| ATOM | 953 | C | LYS | A | 287 | 22.662 | 23.965 | 82.707 | 1.00 | 20.04 | C |
| ATOM | 954 | O | LYS | A | 287 | 23.381 | 23.980 | 83.683 | 1.00 | 20.78 | O |
| ATOM | 968 | N | GLU | A | 288 | 22.743 | 24.877 | 81.764 | 1.00 | 19.81 | N |
| ATOM | 969 | CA | GLU | A | 288 | 23.633 | 26.017 | 81.871 | 1.00 | 20.20 | C |
| ATOM | 970 | CB | GLU | A | 288 | 24.203 | 26.328 | 80.489 | 1.00 | 20.41 | C |
| ATOM | 971 | CG | GLU | A | 288 | 25.680 | 26.039 | 80.413 | 1.00 | 22.77 | C |
| ATOM | 972 | CD | GLU | A | 288 | 25.992 | 24.574 | 80.230 | 1.00 | 24.05 | C |
| ATOM | 973 | OE1 | GLU | A | 288 | 25.508 | 24.003 | 79.236 | 1.00 | 25.33 | O |
| ATOM | 974 | OE2 | GLU | A | 288 | 26.735 | 24.015 | 81.065 | 1.00 | 24.36 | O |
| ATOM | 975 | C | GLU | A | 288 | 22.961 | 27.260 | 82.461 | 1.00 | 19.97 | C |
| ATOM | 976 | O | GLU | A | 288 | 23.617 | 28.150 | 82.967 | 1.00 | 19.12 | O |
| ATOM | 983 | N | LEU | A | 289 | 21.644 | 27.317 | 82.366 | 1.00 | 20.59 | N |
| ATOM | 984 | CA | LEU | A | 289 | 20.865 | 28.403 | 82.940 | 1.00 | 20.93 | C |
| ATOM | 985 | CB | LEU | A | 289 | 19.422 | 28.302 | 82.468 | 1.00 | 21.39 | C |
| ATOM | 986 | CG | LEU | A | 289 | 18.916 | 28.990 | 81.202 | 1.00 | 21.83 | C |
| ATOM | 987 | CD1 | LEU | A | 289 | 20.009 | 29.368 | 80.239 | 1.00 | 23.86 | C |
| ATOM | 988 | CD2 | LEU | A | 289 | 17.850 | 28.090 | 80.569 | 1.00 | 21.58 | C |
| ATOM | 989 | C | LEU | A | 289 | 20.840 | 28.319 | 84.456 | 1.00 | 21.04 | C |
| ATOM | 990 | O | LEU | A | 289 | 21.008 | 27.238 | 85.036 | 1.00 | 21.18 | O |
| ATOM | 1002 | N | GLU | A | 290 | 20.575 | 29.457 | 85.097 | 1.00 | 20.72 | N |
| ATOM | 1003 | CA | GLU | A | 290 | 20.499 | 29.507 | 86.549 | 1.00 | 20.91 | C |
| ATOM | 1004 | CB | GLU | A | 290 | 20.790 | 30.920 | 87.078 | 1.00 | 21.22 | C |
| ATOM | 1005 | CG | GLU | A | 290 | 22.197 | 31.429 | 86.772 | 1.00 | 23.17 | C |
| ATOM | 1006 | CD | GLU | A | 290 | 23.319 | 30.556 | 87.353 | 1.00 | 26.43 | C |
| ATOM | 1007 | OE1 | GLU | A | 290 | 23.204 | 30.106 | 88.517 | 1.00 | 27.12 | O |
| ATOM | 1008 | OE2 | GLU | A | 290 | 24.333 | 30.322 | 86.643 | 1.00 | 29.25 | O |
| ATOM | 1009 | C | GLU | A | 290 | 19.128 | 29.033 | 86.966 | 1.00 | 20.16 | C |
| ATOM | 1010 | O | GLU | A | 290 | 18.209 | 28.997 | 86.163 | 1.00 | 20.19 | O |
| ATOM | 1017 | N | VAL | A | 291 | 18.992 | 28.695 | 88.233 | 1.00 | 19.77 | N |
| ATOM | 1018 | CA | VAL | A | 291 | 17.815 | 27.997 | 88.700 | 1.00 | 20.14 | C |
| ATOM | 1019 | CB | VAL | A | 291 | 17.981 | 27.550 | 90.188 | 1.00 | 20.82 | C |
| ATOM | 1020 | CG1 | VAL | A | 291 | 16.675 | 27.504 | 90.939 | 1.00 | 22.12 | C |
| ATOM | 1021 | CG2 | VAL | A | 291 | 18.611 | 26.157 | 90.239 | 1.00 | 22.69 | C |
| ATOM | 1022 | C | VAL | A | 291 | 16.525 | 28.769 | 88.462 | 1.00 | 18.96 | C |
| ATOM | 1023 | O | VAL | A | 291 | 15.508 | 28.169 | 88.185 | 1.00 | 18.59 | O |
| ATOM | 1033 | N | ALA | A | 292 | 16.587 | 30.092 | 88.536 | 1.00 | 18.33 | N |
| ATOM | 1034 | CA | ALA | A | 292 | 15.400 | 30.928 | 88.404 | 1.00 | 17.83 | C |
| ATOM | 1035 | CB | ALA | A | 292 | 15.735 | 32.391 | 88.720 | 1.00 | 17.74 | C |
| ATOM | 1036 | C | ALA | A | 292 | 14.789 | 30.791 | 87.022 | 1.00 | 17.16 | C |
| ATOM | 1037 | O | ALA | A | 292 | 13.584 | 30.595 | 86.885 | 1.00 | 16.13 | O |
| ATOM | 1043 | N | ASP | A | 293 | 15.641 | 30.845 | 86.008 | 1.00 | 17.07 | N |
| ATOM | 1044 | CA | ASP | A | 293 | 15.215 | 30.638 | 84.622 | 1.00 | 17.08 | C |
| ATOM | 1045 | CB | ASP | A | 293 | 16.343 | 30.948 | 83.645 | 1.00 | 16.87 | C |
| ATOM | 1046 | CG | ASP | A | 293 | 16.479 | 32.426 | 83.366 | 1.00 | 17.26 | C |
| ATOM | 1047 | OD1 | ASP | A | 293 | 15.637 | 33.217 | 83.840 | 1.00 | 19.82 | O |
| ATOM | 1048 | OD2 | ASP | A | 293 | 17.399 | 32.892 | 82.677 | 1.00 | 17.49 | O |
| ATOM | 1049 | C | ASP | A | 293 | 14.719 | 29.233 | 84.380 | 1.00 | 17.08 | C |
| ATOM | 1050 | O | ASP | A | 293 | 13.724 | 29.035 | 83.683 | 1.00 | 16.29 | O |
| ATOM | 1055 | N | GLN | A | 294 | 15.409 | 28.260 | 84.965 | 1.00 | 17.58 | N |
| ATOM | 1056 | CA | GLN | A | 294 | 14.999 | 26.858 | 84.849 | 1.00 | 17.68 | C |
| ATOM | 1057 | CB | GLN | A | 294 | 15.976 | 25.953 | 85.599 | 1.00 | 18.00 | C |
| ATOM | 1058 | CG | GLN | A | 294 | 17.365 | 25.943 | 84.988 | 1.00 | 17.89 | C |
| ATOM | 1059 | CD | GLN | A | 294 | 18.309 | 24.999 | 85.680 | 1.00 | 18.77 | C |
| ATOM | 1060 | OE1 | GLN | A | 294 | 18.176 | 24.878 | 86.972 | 1.00 | 21.65 | O |
| ATOM | 1061 | NE2 | GLN | A | 294 | 19.148 | 24.380 | 85.040 | 1.00 | 17.02 | N |
| ATOM | 1062 | C | GLN | A | 294 | 13.600 | 26.672 | 85.381 | 1.00 | 17.26 | C |
| ATOM | 1063 | O | GLN | A | 294 | 12.818 | 25.912 | 84.834 | 1.00 | 16.86 | O |
| ATOM | 1072 | N | MET | A | 295 | 13.293 | 27.390 | 86.452 | 1.00 | 17.75 | N |
| ATOM | 1073 | CA | MET | A | 295 | 12.004 | 27.263 | 87.092 | 1.00 | 18.05 | C |
| ATOM | 1074 | CB | MET | A | 295 | 12.034 | 27.887 | 88.483 | 1.00 | 18.42 | C |
| ATOM | 1075 | CG | MET | A | 295 | 12.702 | 27.000 | 89.551 | 1.00 | 19.98 | C |
| ATOM | 1076 | SD | MET | A | 295 | 12.655 | 27.635 | 91.234 | 1.00 | 22.36 | S |
| ATOM | 1077 | CE | MET | A | 295 | 13.326 | 29.324 | 91.082 | 1.00 | 22.53 | C |
| ATOM | 1078 | C | MET | A | 295 | 10.909 | 27.882 | 86.227 | 1.00 | 17.10 | C |
| ATOM | 1079 | O | MET | A | 295 | 9.860 | 27.278 | 86.029 | 1.00 | 16.20 | O |
| ATOM | 1089 | N | THR | A | 296 | 11.166 | 29.064 | 85.698 | 1.00 | 16.63 | N |
| ATOM | 1090 | CA | THR | A | 296 | 10.203 | 29.730 | 84.816 | 1.00 | 16.78 | C |
| ATOM | 1091 | CB | THR | A | 296 | 10.761 | 31.073 | 84.333 | 1.00 | 16.68 | C |
| ATOM | 1092 | OG1 | THR | A | 296 | 11.259 | 31.787 | 85.452 | 1.00 | 16.15 | O |
| ATOM | 1093 | CG2 | THR | A | 296 | 9.676 | 31.969 | 83.782 | 1.00 | 16.05 | C |
| ATOM | 1094 | C | THR | A | 296 | 9.833 | 28.871 | 83.623 | 1.00 | 16.80 | C |
| ATOM | 1095 | O | THR | A | 296 | 8.650 | 28.694 | 83.314 | 1.00 | 16.59 | O |
| ATOM | 1103 | N | LEU | A | 297 | 10.854 | 28.328 | 82.970 | 1.00 | 17.38 | N |
| ATOM | 1104 | CA | LEU | A | 297 | 10.680 | 27.486 | 81.798 | 1.00 | 17.65 | C |
| ATOM | 1105 | CB | LEU | A | 297 | 12.025 | 27.002 | 81.297 | 1.00 | 18.16 | C |
| ATOM | 1106 | CG | LEU | A | 297 | 12.962 | 27.998 | 80.623 | 1.00 | 18.62 | C |
| ATOM | 1107 | CD1 | LEU | A | 297 | 14.275 | 27.323 | 80.349 | 1.00 | 19.93 | C |
| ATOM | 1108 | CD2 | LEU | A | 297 | 12.355 | 28.481 | 79.347 | 1.00 | 20.09 | C |
| ATOM | 1109 | C | LEU | A | 297 | 9.832 | 26.276 | 82.088 | 1.00 | 18.35 | C |
| ATOM | 1110 | O | LEU | A | 297 | 8.915 | 25.962 | 81.329 | 1.00 | 19.02 | O |
| ATOM | 1122 | N | LEU | A | 298 | 10.142 | 25.599 | 83.189 | 1.00 | 18.67 | N |
| ATOM | 1123 | CA | LEU | A | 298 | 9.397 | 24.419 | 83.616 | 1.00 | 19.13 | C |
| ATOM | 1124 | CB | LEU | A | 298 | 10.178 | 23.634 | 84.664 | 1.00 | 19.15 | C |
| ATOM | 1125 | CG | LEU | A | 298 | 11.265 | 22.722 | 84.080 | 1.00 | 18.78 | C |
| ATOM | 1126 | CD1 | LEU | A | 298 | 12.047 | 22.108 | 85.222 | 1.00 | 19.04 | C |
| ATOM | 1127 | CD2 | LEU | A | 298 | 10.676 | 21.659 | 83.196 | 1.00 | 17.14 | C |
| ATOM | 1128 | C | LEU | A | 298 | 7.995 | 24.730 | 84.134 | 1.00 | 19.11 | C |
| ATOM | 1129 | O | LEU | A | 298 | 7.082 | 23.934 | 83.908 | 1.00 | 18.71 | O |
| ATOM | 1141 | N | GLN | A | 299 | 7.853 | 25.868 | 84.825 | 1.00 | 19.24 | N |
| ATOM | 1142 | CA | GLN | A | 299 | 6.554 | 26.427 | 85.209 | 1.00 | 19.28 | C |
| ATOM | 1143 | CB | GLN | A | 299 | 6.739 | 27.671 | 86.094 | 1.00 | 19.30 | C |
| ATOM | 1144 | CG | GLN | A | 299 | 5.502 | 28.155 | 86.860 | 1.00 | 20.58 | C |
| ATOM | 1145 | CD | GLN | A | 299 | 4.979 | 27.176 | 87.966 | 1.00 | 23.78 | C |
| ATOM | 1146 | OE1 | GLN | A | 299 | 5.757 | 26.994 | 89.041 | 1.00 | 25.06 | O |
| ATOM | 1147 | NE2 | GLN | A | 299 | 3.873 | 26.632 | 87.849 | 1.00 | 24.01 | N |
| ATOM | 1148 | C | GLN | A | 299 | 5.673 | 26.737 | 83.979 | 1.00 | 19.23 | C |
| ATOM | 1149 | O | GLN | A | 299 | 4.472 | 26.540 | 84.018 | 1.00 | 19.08 | O |
| ATOM | 1158 | N | ASN | A | 300 | 6.281 | 27.140 | 82.877 | 1.00 | 19.52 | N |
| ATOM | 1159 | CA | ASN | A | 300 | 5.558 | 27.383 | 81.638 | 1.00 | 19.92 | C |
| ATOM | 1160 | CB | ASN | A | 300 | 6.390 | 28.268 | 80.717 | 1.00 | 20.02 | C |
| ATOM | 1161 | CG | ASN | A | 300 | 5.666 | 28.610 | 79.428 | 1.00 | 22.82 | C |
| ATOM | 1162 | OD1 | ASN | A | 300 | 4.638 | 29.280 | 79.465 | 1.00 | 26.46 | O |
| ATOM | 1163 | ND2 | ASN | A | 300 | 6.203 | 28.154 | 78.273 | 1.00 | 23.27 | N |
| ATOM | 1164 | C | ASN | A | 300 | 5.132 | 26.126 | 80.878 | 1.00 | 20.03 | C |
| ATOM | 1165 | O | ASN | A | 300 | 4.150 | 26.156 | 80.172 | 1.00 | 19.93 | O |
| ATOM | 1172 | N | CYS | A | 301 | 5.854 | 25.023 | 81.010 | 1.00 | 20.59 | N |
| ATOM | 1173 | CA | CYS | A | 301 | 5.672 | 23.913 | 80.087 | 1.00 | 21.15 | C |
| ATOM | 1174 | CB | CYS | A | 301 | 6.894 | 23.801 | 79.168 | 1.00 | 20.97 | C |
| ATOM | 1175 | SG | CYS | A | 301 | 8.353 | 23.046 | 79.875 | 1.00 | 21.58 | S |
| ATOM | 1176 | C | CYS | A | 301 | 5.349 | 22.560 | 80.694 | 1.00 | 21.23 | C |
| ATOM | 1177 | O | CYS | A | 301 | 5.186 | 21.592 | 79.965 | 1.00 | 22.06 | O |
| ATOM | 1183 | N | TRP | A | 302 | 5.220 | 22.489 | 82.009 | 1.00 | 21.11 | N |
| ATOM | 1184 | CA | TRP | A | 302 | 5.165 | 21.196 | 82.678 | 1.00 | 20.70 | C |
| ATOM | 1185 | CB | TRP | A | 302 | 5.130 | 21.360 | 84.208 | 1.00 | 20.50 | C |
| ATOM | 1186 | CG | TRP | A | 302 | 3.951 | 22.097 | 84.721 | 1.00 | 19.38 | C |
| ATOM | 1187 | CD1 | TRP | A | 302 | 3.830 | 23.440 | 84.864 | 1.00 | 18.06 | C |
| ATOM | 1188 | NE1 | TRP | A | 302 | 2.597 | 23.747 | 85.365 | 1.00 | 16.25 | N |
| ATOM | 1189 | CE2 | TRP | A | 302 | 1.888 | 22.594 | 85.562 | 1.00 | 17.26 | C |
| ATOM | 1190 | CD2 | TRP | A | 302 | 2.719 | 21.529 | 85.192 | 1.00 | 17.84 | C |
| ATOM | 1191 | CE3 | TRP | A | 302 | 2.226 | 20.217 | 85.309 | 1.00 | 18.15 | C |
| ATOM | 1192 | CZ3 | TRP | A | 302 | 0.959 | 20.026 | 85.793 | 1.00 | 17.88 | C |
| ATOM | 1193 | CH2 | TRP | A | 302 | 0.167 | 21.115 | 86.171 | 1.00 | 19.50 | C |
| ATOM | 1194 | CZ2 | TRP | A | 302 | 0.616 | 22.404 | 86.069 | 1.00 | 17.35 | C |
| ATOM | 1195 | C | TRP | A | 302 | 3.979 | 20.355 | 82.221 | 1.00 | 20.85 | C |
| ATOM | 1196 | O | TRP | A | 302 | 4.110 | 19.150 | 82.056 | 1.00 | 20.52 | O |
| ATOM | 1207 | N | SER | A | 303 | 2.826 | 20.985 | 82.010 | 1.00 | 20.60 | N |
| ATOM | 1208 | CA | SER | A | 303 | 1.637 | 20.237 | 81.637 | 1.00 | 20.95 | C |
| ATOM | 1209 | CB | SER | A | 303 | 0.350 | 21.037 | 81.924 | 1.00 | 20.45 | C |
| ATOM | 1210 | OG | SER | A | 303 | 0.293 | 22.224 | 81.170 | 1.00 | 22.98 | O |
| ATOM | 1211 | C | SER | A | 303 | 1.721 | 19.775 | 80.182 | 1.00 | 20.51 | C |
| ATOM | 1212 | O | SER | A | 303 | 1.297 | 18.675 | 79.850 | 1.00 | 20.40 | O |
| ATOM | 1218 | N | GLU | A | 304 | 2.314 | 20.613 | 79.338 | 1.00 | 20.46 | N |
| ATOM | 1219 | CA | GLU | A | 304 | 2.607 | 20.288 | 77.936 | 1.00 | 20.75 | C |
| ATOM | 1220 | CB | GLU | A | 304 | 3.217 | 21.495 | 77.216 | 1.00 | 21.39 | C |
| ATOM | 1221 | CG | GLU | A | 304 | 2.241 | 22.636 | 77.022 | 1.00 | 24.42 | C |
| ATOM | 1222 | CD | GLU | A | 304 | 2.145 | 23.630 | 78.193 | 1.00 | 30.67 | C |
| ATOM | 1223 | OE1 | GLU | A | 304 | 2.592 | 23.352 | 79.347 | 1.00 | 32.15 | O |
| ATOM | 1224 | OE2 | GLU | A | 304 | 1.549 | 24.715 | 77.975 | 1.00 | 35.31 | O |
| ATOM | 1225 | C | GLU | A | 304 | 3.553 | 19.121 | 77.813 | 1.00 | 19.84 | C |
| ATOM | 1226 | O | GLU | A | 304 | 3.320 | 18.225 | 77.032 | 1.00 | 19.95 | O |
| ATOM | 1233 | N | LEU | A | 305 | 4.606 | 19.102 | 78.617 | 1.00 | 20.17 | N |
| ATOM | 1234 | CA | LEU | A | 305 | 5.529 | 17.980 | 78.593 | 1.00 | 20.25 | C |
| ATOM | 1235 | CB | LEU | A | 305 | 6.766 | 18.294 | 79.438 | 1.00 | 20.34 | C |
| ATOM | 1236 | CG | LEU | A | 305 | 7.777 | 19.285 | 78.844 | 1.00 | 20.25 | C |
| ATOM | 1237 | CD1 | LEU | A | 305 | 8.844 | 19.587 | 79.833 | 1.00 | 20.74 | C |
| ATOM | 1238 | CD2 | LEU | A | 305 | 8.404 | 18.759 | 77.591 | 1.00 | 21.19 | C |
| ATOM | 1239 | C | LEU | A | 305 | 4.894 | 16.645 | 79.029 | 1.00 | 20.69 | C |
| ATOM | 1240 | O | LEU | A | 305 | 5.220 | 15.591 | 78.483 | 1.00 | 20.86 | O |
| ATOM | 1252 | N | LEU | A | 306 | 4.020 | 16.696 | 80.028 | 1.00 | 21.12 | N |
| ATOM | 1253 | CA | LEU | A | 306 | 3.309 | 15.519 | 80.499 | 1.00 | 21.35 | C |
| ATOM | 1254 | CB | LEU | A | 306 | 2.593 | 15.835 | 81.803 | 1.00 | 21.80 | C |
| ATOM | 1255 | CG | LEU | A | 306 | 3.372 | 15.569 | 83.092 | 1.00 | 23.77 | C |
| ATOM | 1256 | CD1 | LEU | A | 306 | 2.756 | 16.346 | 84.260 | 1.00 | 24.44 | C |
| ATOM | 1257 | CD2 | LEU | A | 306 | 3.455 | 14.057 | 83.423 | 1.00 | 23.19 | C |
| ATOM | 1258 | C | LEU | A | 306 | 2.299 | 14.994 | 79.484 | 1.00 | 21.24 | C |
| ATOM | 1259 | O | LEU | A | 306 | 2.223 | 13.799 | 79.239 | 1.00 | 21.44 | O |
| ATOM | 1271 | N | VAL | A | 307 | 1.514 | 15.890 | 78.904 | 1.00 | 21.57 | N |
| ATOM | 1272 | CA | VAL | A | 307 | 0.588 | 15.530 | 77.822 | 1.00 | 21.86 | C |
| ATOM | 1273 | CB | VAL | A | 307 | −0.268 | 16.784 | 77.355 | 1.00 | 22.01 | C |
| ATOM | 1274 | CG1 | VAL | A | 307 | −0.963 | 16.532 | 76.027 | 1.00 | 21.89 | C |
| ATOM | 1275 | CG2 | VAL | A | 307 | −1.281 | 17.208 | 78.413 | 1.00 | 22.12 | C |
| ATOM | 1276 | C | VAL | A | 307 | 1.348 | 14.936 | 76.617 | 1.00 | 21.69 | C |
| ATOM | 1277 | O | VAL | A | 307 | 0.987 | 13.881 | 76.094 | 1.00 | 21.37 | O |
| ATOM | 1287 | N | PHE | A | 308 | 2.409 | 15.614 | 76.196 | 1.00 | 21.54 | N |
| ATOM | 1288 | CA | PHE | A | 308 | 3.206 | 15.138 | 75.096 | 1.00 | 21.47 | C |
| ATOM | 1289 | CB | PHE | A | 308 | 4.268 | 16.163 | 74.685 | 1.00 | 21.49 | C |
| ATOM | 1290 | CG | PHE | A | 308 | 4.924 | 15.862 | 73.334 | 1.00 | 21.66 | C |
| ATOM | 1291 | CD1 | PHE | A | 308 | 6.321 | 15.852 | 73.198 | 1.00 | 21.16 | C |
| ATOM | 1292 | CE1 | PHE | A | 308 | 6.929 | 15.612 | 71.992 | 1.00 | 21.59 | C |
| ATOM | 1293 | CZ | PHE | A | 308 | 6.164 | 15.366 | 70.870 | 1.00 | 21.69 | C |
| ATOM | 1294 | CE2 | PHE | A | 308 | 4.768 | 15.363 | 70.973 | 1.00 | 23.31 | C |
| ATOM | 1295 | CD2 | PHE | A | 308 | 4.150 | 15.608 | 72.203 | 1.00 | 22.65 | C |
| ATOM | 1296 | C | PHE | A | 308 | 3.854 | 13.807 | 75.434 | 1.00 | 21.64 | C |
| ATOM | 1297 | O | PHE | A | 308 | 3.973 | 12.942 | 74.574 | 1.00 | 22.04 | O |
| ATOM | 1307 | N | ASP | A | 309 | 4.257 | 13.626 | 76.681 | 1.00 | 21.68 | N |
| ATOM | 1308 | CA | ASP | A | 309 | 4.782 | 12.350 | 77.103 | 1.00 | 21.58 | C |
| ATOM | 1309 | CB | ASP | A | 309 | 5.241 | 12.414 | 78.555 | 1.00 | 22.13 | C |
| ATOM | 1310 | CG | ASP | A | 309 | 5.889 | 11.124 | 79.003 | 1.00 | 23.04 | C |
| ATOM | 1311 | OD1 | ASP | A | 309 | 5.239 | 10.310 | 79.730 | 1.00 | 24.78 | O |
| ATOM | 1312 | OD2 | ASP | A | 309 | 7.042 | 10.834 | 78.625 | 1.00 | 26.05 | O |
| ATOM | 1313 | C | ASP | A | 309 | 3.741 | 11.233 | 76.942 | 1.00 | 21.24 | C |
| ATOM | 1314 | O | ASP | A | 309 | 4.056 | 10.118 | 76.481 | 1.00 | 21.30 | O |
| ATOM | 1319 | N | HIS | A | 310 | 2.513 | 11.533 | 77.332 | 1.00 | 20.67 | N |
| ATOM | 1320 | CA | HIS | A | 310 | 1.438 | 10.569 | 77.254 | 1.00 | 20.41 | C |
| ATOM | 1321 | CB | HIS | A | 310 | 0.230 | 11.073 | 78.071 | 1.00 | 20.28 | C |
| ATOM | 1322 | CG | HIS | A | 310 | −1.065 | 10.444 | 77.687 | 1.00 | 21.06 | C |
| ATOM | 1323 | ND1 | HIS | A | 310 | −1.279 | 9.085 | 77.748 | 1.00 | 22.09 | N |
| ATOM | 1324 | CE1 | HIS | A | 310 | −2.497 | 8.812 | 77.315 | 1.00 | 22.28 | C |
| ATOM | 1325 | NE2 | HIS | A | 310 | −3.082 | 9.943 | 76.974 | 1.00 | 22.19 | N |
| ATOM | 1326 | CD2 | HIS | A | 310 | −2.207 | 10.981 | 77.201 | 1.00 | 22.28 | C |
| ATOM | 1327 | C | HIS | A | 310 | 1.099 | 10.270 | 75.790 | 1.00 | 20.35 | C |
| ATOM | 1328 | O | HIS | A | 310 | 1.022 | 9.104 | 75.408 | 1.00 | 20.75 | O |
| ATOM | 1337 | N | ILE | A | 311 | 0.945 | 11.317 | 74.981 | 1.00 | 20.51 | N |
| ATOM | 1338 | CA | ILE | A | 311 | 0.706 | 11.225 | 73.525 | 1.00 | 20.82 | C |
| ATOM | 1339 | CB | ILE | A | 311 | 0.764 | 12.646 | 72.884 | 1.00 | 21.25 | C |
| ATOM | 1340 | CG1 | ILE | A | 311 | −0.381 | 13.560 | 73.368 | 1.00 | 22.39 | C |
| ATOM | 1341 | CD1 | ILE | A | 311 | −1.777 | 13.051 | 73.060 | 1.00 | 24.52 | C |
| ATOM | 1342 | CG2 | ILE | A | 311 | 0.752 | 12.576 | 71.374 | 1.00 | 20.33 | C |
| ATOM | 1343 | C | ILE | A | 311 | 1.713 | 10.325 | 72.780 | 1.00 | 21.18 | C |
| ATOM | 1344 | O | ILE | A | 311 | 1.326 | 9.410 | 72.040 | 1.00 | 20.68 | O |
| ATOM | 1356 | N | TYR | A | 312 | 3.004 | 10.584 | 72.970 | 1.00 | 21.76 | N |
| ATOM | 1357 | CA | TYR | A | 312 | 4.028 | 9.813 | 72.276 | 1.00 | 22.63 | C |
| ATOM | 1358 | CB | TYR | A | 312 | 5.437 | 10.389 | 72.473 | 1.00 | 22.96 | C |
| ATOM | 1359 | CG | TYR | A | 312 | 6.451 | 9.638 | 71.629 | 1.00 | 22.71 | C |
| ATOM | 1360 | CD1 | TYR | A | 312 | 6.299 | 9.559 | 70.267 | 1.00 | 22.87 | C |
| ATOM | 1361 | CE1 | TYR | A | 312 | 7.199 | 8.868 | 69.477 | 1.00 | 23.85 | C |
| ATOM | 1362 | CZ | TYR | A | 312 | 8.269 | 8.217 | 70.049 | 1.00 | 24.74 | C |
| ATOM | 1363 | OH | TYR | A | 312 | 9.139 | 7.517 | 69.215 | 1.00 | 26.56 | O |
| ATOM | 1364 | CE2 | TYR | A | 312 | 8.444 | 8.260 | 71.425 | 1.00 | 23.16 | C |
| ATOM | 1365 | CD2 | TYR | A | 312 | 7.532 | 8.970 | 72.204 | 1.00 | 24.17 | C |
| ATOM | 1366 | C | TYR | A | 312 | 4.018 | 8.351 | 72.686 | 1.00 | 23.09 | C |
| ATOM | 1367 | O | TYR | A | 312 | 4.278 | 7.470 | 71.854 | 1.00 | 23.34 | O |
| ATOM | 1377 | N | ARG | A | 313 | 3.711 | 8.100 | 73.959 | 1.00 | 23.46 | N |
| ATOM | 1378 | CA | ARG | A | 313 | 3.463 | 6.745 | 74.440 | 1.00 | 23.34 | C |
| ATOM | 1379 | CB | ARG | A | 313 | 3.058 | 6.758 | 75.918 | 1.00 | 23.46 | C |
| ATOM | 1380 | CG | ARG | A | 313 | 2.996 | 5.368 | 76.573 | 1.00 | 22.66 | C |
| ATOM | 1381 | CD | ARG | A | 313 | 3.005 | 5.399 | 78.082 | 1.00 | 20.99 | C |
| ATOM | 1382 | NE | ARG | A | 313 | 4.263 | 5.946 | 78.577 | 1.00 | 21.01 | N |
| ATOM | 1383 | CZ | ARG | A | 313 | 4.458 | 7.168 | 79.086 | 1.00 | 20.05 | C |
| ATOM | 1384 | NH1 | ARG | A | 313 | 3.468 | 8.039 | 79.218 | 1.00 | 18.83 | N |
| ATOM | 1385 | NH2 | ARG | A | 313 | 5.687 | 7.503 | 79.473 | 1.00 | 21.35 | N |
| ATOM | 1386 | C | ARG | A | 313 | 2.374 | 6.053 | 73.630 | 1.00 | 23.31 | C |
| ATOM | 1387 | O | ARG | A | 313 | 2.478 | 4.853 | 73.340 | 1.00 | 23.93 | O |
| ATOM | 1401 | N | GLN | A | 314 | 1.340 | 6.804 | 73.268 | 1.00 | 23.29 | N |
| ATOM | 1402 | CA | GLN | A | 314 | 0.239 | 6.258 | 72.480 | 1.00 | 23.40 | C |
| ATOM | 1403 | CB | GLN | A | 314 | −0.998 | 7.169 | 72.526 | 1.00 | 23.36 | C |
| ATOM | 1404 | CG | GLN | A | 314 | −1.562 | 7.494 | 73.918 | 1.00 | 22.48 | C |
| ATOM | 1405 | CD | GLN | A | 314 | −1.328 | 6.407 | 74.925 | 1.00 | 20.80 | C |
| ATOM | 1406 | OE1 | GLN | A | 314 | −1.853 | 5.232 | 74.649 | 1.00 | 21.43 | O |
| ATOM | 1407 | NE2 | GLN | A | 314 | −0.676 | 6.628 | 75.942 | 1.00 | 19.01 | N |
| ATOM | 1408 | C | GLN | A | 314 | 0.618 | 6.026 | 71.033 | 1.00 | 23.80 | C |
| ATOM | 1409 | O | GLN | A | 314 | 0.028 | 5.165 | 70.394 | 1.00 | 24.46 | O |
| ATOM | 1418 | N | VAL | A | 315 | 1.572 | 6.775 | 70.488 | 1.00 | 23.99 | N |
| ATOM | 1419 | CA | VAL | A | 315 | 1.980 | 6.489 | 69.110 | 1.00 | 24.72 | C |
| ATOM | 1420 | CB | VAL | A | 315 | 2.621 | 7.719 | 68.306 | 1.00 | 24.77 | C |
| ATOM | 1421 | CG1 | VAL | A | 315 | 2.059 | 9.047 | 68.744 | 1.00 | 23.75 | C |
| ATOM | 1422 | CG2 | VAL | A | 315 | 4.164 | 7.696 | 68.319 | 1.00 | 25.94 | C |
| ATOM | 1423 | C | VAL | A | 315 | 2.853 | 5.219 | 69.097 | 1.00 | 24.57 | C |
| ATOM | 1424 | O | VAL | A | 315 | 2.744 | 4.401 | 68.192 | 1.00 | 24.65 | O |
| ATOM | 1434 | N | GLN | A | 316 | 3.664 | 5.052 | 70.143 | 1.00 | 24.61 | N |
| ATOM | 1435 | CA | GLN | A | 316 | 4.443 | 3.834 | 70.368 | 1.00 | 24.30 | C |
| ATOM | 1436 | CB | GLN | A | 316 | 5.307 | 3.997 | 71.605 | 1.00 | 24.38 | C |
| ATOM | 1437 | CG | GLN | A | 316 | 6.499 | 4.860 | 71.385 | 1.00 | 25.28 | C |
| ATOM | 1438 | CD | GLN | A | 316 | 7.358 | 4.988 | 72.630 | 1.00 | 27.40 | C |
| ATOM | 1439 | OE1 | GLN | A | 316 | 6.872 | 5.435 | 73.690 | 1.00 | 29.49 | O |
| ATOM | 1440 | NE2 | GLN | A | 316 | 8.640 | 4.605 | 72.515 | 1.00 | 26.39 | N |
| ATOM | 1441 | C | GLN | A | 316 | 3.585 | 2.604 | 70.578 | 1.00 | 24.20 | C |
| ATOM | 1442 | O | GLN | A | 316 | 4.026 | 1.492 | 70.320 | 1.00 | 24.57 | O |
| ATOM | 1451 | N | HIS | A | 317 | 2.370 | 2.810 | 71.075 | 1.00 | 24.08 | N |
| ATOM | 1452 | CA | HIS | A | 317 | 1.456 | 1.732 | 71.448 | 1.00 | 23.73 | C |
| ATOM | 1453 | CB | HIS | A | 317 | 0.416 | 2.304 | 72.421 | 1.00 | 23.53 | C |
| ATOM | 1454 | CG | HIS | A | 317 | −0.573 | 1.302 | 72.926 | 1.00 | 23.31 | C |
| ATOM | 1455 | ND1 | HIS | A | 317 | −0.444 | −0.009 | 73.236 | 1.00 | 23.30 | N |
| ATOM | 1456 | CE1 | HIS | A | 317 | −1.671 | −0.444 | 73.664 | 1.00 | 21.87 | C |
| ATOM | 1457 | NE2 | HIS | A | 317 | −2.523 | 0.562 | 73.625 | 1.00 | 21.83 | N |
| ATOM | 1458 | CD2 | HIS | A | 317 | −1.884 | 1.631 | 73.191 | 1.00 | 22.24 | C |
| ATOM | 1459 | C | HIS | A | 317 | 0.789 | 1.156 | 70.188 | 1.00 | 23.84 | C |
| ATOM | 1460 | O | HIS | A | 317 | 0.771 | −0.070 | 69.948 | 1.00 | 23.41 | O |
| ATOM | 1469 | N | GLY | A | 318 | 0.259 | 2.075 | 69.384 | 1.00 | 24.10 | N |
| ATOM | 1470 | CA | GLY | A | 318 | −0.301 | 1.759 | 68.092 | 1.00 | 24.13 | C |
| ATOM | 1471 | C | GLY | A | 318 | −1.578 | 0.962 | 68.186 | 1.00 | 24.12 | C |
| ATOM | 1472 | O | GLY | A | 318 | −1.838 | 0.134 | 67.304 | 1.00 | 24.08 | O |
| ATOM | 1476 | N | LYS | A | 319 | −2.363 | 1.200 | 69.244 | 1.00 | 24.19 | N |
| ATOM | 1477 | CA | LYS | A | 319 | −3.687 | 0.580 | 69.393 | 1.00 | 24.31 | C |
| ATOM | 1478 | CB | LYS | A | 319 | −3.700 | −0.473 | 70.504 | 1.00 | 24.38 | C |
| ATOM | 1479 | CG | LYS | A | 319 | −2.494 | −1.401 | 70.532 | 1.00 | 24.18 | C |
| ATOM | 1480 | CD | LYS | A | 319 | −2.727 | −2.546 | 71.507 | 1.00 | 24.92 | C |
| ATOM | 1481 | CE | LYS | A | 319 | −1.430 | −3.266 | 71.900 | 1.00 | 25.47 | C |
| ATOM | 1482 | NZ | LYS | A | 319 | −1.709 | −4.534 | 72.674 | 1.00 | 25.92 | N |
| ATOM | 1483 | C | LYS | A | 319 | −4.774 | 1.623 | 69.665 | 1.00 | 24.39 | C |
| ATOM | 1484 | O | LYS | A | 319 | −4.726 | 2.352 | 70.652 | 1.00 | 24.14 | O |
| ATOM | 1498 | N | GLU | A | 320 | −5.759 | 1.667 | 68.775 | 1.00 | 24.48 | N |
| ATOM | 1499 | CA | GLU | A | 320 | −6.871 | 2.604 | 68.879 | 1.00 | 24.63 | C |
| ATOM | 1500 | CB | GLU | A | 320 | −7.634 | 2.651 | 67.540 | 1.00 | 24.85 | C |
| ATOM | 1501 | CG | GLU | A | 320 | −8.295 | 3.998 | 67.275 | 1.00 | 26.39 | C |
| ATOM | 1502 | CD | GLU | A | 320 | −8.683 | 4.215 | 65.834 | 1.00 | 27.60 | C |
| ATOM | 1503 | OE1 | GLU | A | 320 | −8.842 | 3.211 | 65.111 | 1.00 | 30.80 | O |
| ATOM | 1504 | OE2 | GLU | A | 320 | −8.833 | 5.388 | 65.431 | 1.00 | 27.73 | O |
| ATOM | 1505 | C | GLU | A | 320 | −7.847 | 2.262 | 70.020 | 1.00 | 24.25 | C |
| ATOM | 1506 | O | GLU | A | 320 | −8.579 | 3.140 | 70.507 | 1.00 | 24.05 | O |
| ATOM | 1513 | N | GLY | A | 321 | −7.839 | 0.996 | 70.445 | 1.00 | 23.69 | N |
| ATOM | 1514 | CA | GLY | A | 321 | −8.807 | 0.496 | 71.403 | 1.00 | 23.31 | C |
| ATOM | 1515 | C | GLY | A | 321 | −8.430 | 0.663 | 72.863 | 1.00 | 22.99 | C |
| ATOM | 1516 | O | GLY | A | 321 | −9.261 | 0.418 | 73.721 | 1.00 | 22.56 | O |
| ATOM | 1520 | N | SER | A | 322 | −7.193 | 1.075 | 73.147 | 1.00 | 23.06 | N |
| ATOM | 1521 | CA | SER | A | 322 | −6.719 | 1.202 | 74.526 | 1.00 | 22.78 | C |
| ATOM | 1522 | CB | SER | A | 322 | −6.270 | −0.155 | 75.068 | 1.00 | 22.77 | C |
| ATOM | 1523 | OG | SER | A | 322 | −5.114 | −0.629 | 74.412 | 1.00 | 22.08 | O |
| ATOM | 1524 | C | SER | A | 322 | −5.590 | 2.203 | 74.701 | 1.00 | 23.07 | C |
| ATOM | 1525 | O | SER | A | 322 | −4.966 | 2.651 | 73.723 | 1.00 | 23.00 | O |
| ATOM | 1531 | N | ILE | A | 323 | −5.336 | 2.537 | 75.963 | 1.00 | 23.05 | N |
| ATOM | 1532 | CA | ILE | A | 323 | −4.311 | 3.507 | 76.353 | 1.00 | 23.52 | C |
| ATOM | 1533 | CB | ILE | A | 323 | −4.886 | 4.577 | 77.318 | 1.00 | 24.12 | C |
| ATOM | 1534 | CG1 | ILE | A | 323 | −5.976 | 5.409 | 76.648 | 1.00 | 24.89 | C |
| ATOM | 1535 | CD1 | ILE | A | 323 | −5.496 | 6.209 | 75.497 | 1.00 | 24.58 | C |
| ATOM | 1536 | CG2 | ILE | A | 323 | −3.801 | 5.498 | 77.810 | 1.00 | 26.10 | C |
| ATOM | 1537 | C | ILE | A | 323 | −3.207 | 2.766 | 77.073 | 1.00 | 22.71 | C |
| ATOM | 1538 | O | ILE | A | 323 | −3.467 | 1.914 | 77.898 | 1.00 | 21.81 | O |
| ATOM | 1550 | N | LEU | A | 324 | −1.972 | 3.124 | 76.765 | 1.00 | 22.29 | N |
| ATOM | 1551 | CA | LEU | A | 324 | −0.822 | 2.597 | 77.460 | 1.00 | 21.87 | C |
| ATOM | 1552 | CB | LEU | A | 324 | 0.312 | 2.327 | 76.469 | 1.00 | 21.84 | C |
| ATOM | 1553 | CG | LEU | A | 324 | 1.278 | 1.155 | 76.651 | 1.00 | 22.02 | C |
| ATOM | 1554 | CD1 | LEU | A | 324 | 2.677 | 1.682 | 76.882 | 1.00 | 23.05 | C |
| ATOM | 1555 | CD2 | LEU | A | 324 | 0.895 | 0.218 | 77.770 | 1.00 | 21.56 | C |
| ATOM | 1556 | C | LEU | A | 324 | −0.394 | 3.619 | 78.507 | 1.00 | 21.45 | C |
| ATOM | 1557 | O | LEU | A | 324 | −0.317 | 4.835 | 78.246 | 1.00 | 21.42 | O |
| ATOM | 1569 | N | LEU | A | 325 | −0.124 | 3.101 | 79.698 | 1.00 | 21.12 | N |
| ATOM | 1570 | CA | LEU | A | 325 | 0.419 | 3.882 | 80.773 | 1.00 | 20.88 | C |
| ATOM | 1571 | CB | LEU | A | 325 | −0.301 | 3.577 | 82.069 | 1.00 | 21.12 | C |
| ATOM | 1572 | CG | LEU | A | 325 | −1.820 | 3.613 | 82.049 | 1.00 | 21.55 | C |
| ATOM | 1573 | CD1 | LEU | A | 325 | −2.332 | 3.276 | 83.439 | 1.00 | 20.62 | C |
| ATOM | 1574 | CD2 | LEU | A | 325 | −2.308 | 4.977 | 81.611 | 1.00 | 21.94 | C |
| ATOM | 1575 | C | LEU | A | 325 | 1.871 | 3.528 | 80.935 | 1.00 | 20.52 | C |
| ATOM | 1576 | O | LEU | A | 325 | 2.323 | 2.470 | 80.508 | 1.00 | 19.16 | O |
| ATOM | 1588 | N | VAL | A | 326 | 2.578 | 4.441 | 81.586 | 1.00 | 20.80 | N |
| ATOM | 1589 | CA | VAL | A | 326 | 4.003 | 4.326 | 81.809 | 1.00 | 21.24 | C |
| ATOM | 1590 | CB | VAL | A | 326 | 4.526 | 5.631 | 82.452 | 1.00 | 21.24 | C |
| ATOM | 1591 | CG1 | VAL | A | 326 | 3.965 | 5.829 | 83.848 | 1.00 | 21.63 | C |
| ATOM | 1592 | CG2 | VAL | A | 326 | 6.057 | 5.667 | 82.442 | 1.00 | 22.07 | C |
| ATOM | 1593 | C | VAL | A | 326 | 4.395 | 3.054 | 82.612 | 1.00 | 21.65 | C |
| ATOM | 1594 | O | VAL | A | 326 | 5.506 | 2.502 | 82.448 | 1.00 | 21.72 | O |
| ATOM | 1604 | N | THR | A | 327 | 3.454 | 2.573 | 83.431 | 1.00 | 21.61 | N |
| ATOM | 1605 | CA | THR | A | 327 | 3.605 | 1.333 | 84.187 | 1.00 | 21.24 | C |
| ATOM | 1606 | CB | THR | A | 327 | 2.472 | 1.191 | 85.244 | 1.00 | 21.56 | C |
| ATOM | 1607 | OG1 | THR | A | 327 | 1.195 | 1.095 | 84.595 | 1.00 | 20.95 | O |
| ATOM | 1608 | CG2 | THR | A | 327 | 2.373 | 2.427 | 86.153 | 1.00 | 21.07 | C |
| ATOM | 1609 | C | THR | A | 327 | 3.572 | 0.073 | 83.334 | 1.00 | 21.09 | C |
| ATOM | 1610 | O | THR | A | 327 | 3.859 | −0.991 | 83.835 | 1.00 | 21.10 | O |
| ATOM | 1618 | N | GLY | A | 328 | 3.192 | 0.189 | 82.069 | 1.00 | 21.05 | N |
| ATOM | 1619 | CA | GLY | A | 328 | 2.989 | −0.956 | 81.209 | 1.00 | 21.08 | C |
| ATOM | 1620 | C | GLY | A | 328 | 1.537 | −1.369 | 81.115 | 1.00 | 21.18 | C |
| ATOM | 1621 | O | GLY | A | 328 | 1.188 | −2.259 | 80.359 | 1.00 | 21.32 | O |
| ATOM | 1625 | N | GLN | A | 329 | 0.680 | −0.707 | 81.868 | 1.00 | 21.78 | N |
| ATOM | 1626 | CA | GLN | A | 329 | −0.715 | −1.092 | 81.955 | 1.00 | 22.16 | C |
| ATOM | 1627 | CB | GLN | A | 329 | −1.289 | −0.585 | 83.272 | 1.00 | 22.57 | C |
| ATOM | 1628 | CG | GLN | A | 329 | −2.614 | −1.200 | 83.659 | 1.00 | 23.66 | C |
| ATOM | 1629 | CD | GLN | A | 329 | −3.370 | −0.371 | 84.695 | 1.00 | 25.91 | C |
| ATOM | 1630 | OE1 | GLN | A | 329 | −2.797 | 0.514 | 85.366 | 1.00 | 27.12 | O |
| ATOM | 1631 | NE2 | GLN | A | 329 | −4.659 | −0.652 | 84.829 | 1.00 | 26.09 | N |
| ATOM | 1632 | C | GLN | A | 329 | −1.485 | −0.485 | 80.811 | 1.00 | 22.00 | C |
| ATOM | 1633 | O | GLN | A | 329 | −1.285 | 0.662 | 80.502 | 1.00 | 22.16 | O |
| ATOM | 1642 | N | GLU | A | 330 | −2.368 | −1.256 | 80.188 | 1.00 | 22.41 | N |
| ATOM | 1643 | CA | GLU | A | 330 | −3.260 | −0.751 | 79.143 | 1.00 | 22.85 | C |
| ATOM | 1644 | CB | GLU | A | 330 | −3.288 | −1.686 | 77.922 | 1.00 | 22.97 | C |
| ATOM | 1645 | CG | GLU | A | 330 | −1.916 | −2.100 | 77.400 | 1.00 | 23.69 | C |
| ATOM | 1646 | CD | GLU | A | 330 | −1.968 | −2.989 | 76.170 | 1.00 | 24.58 | C |
| ATOM | 1647 | OE1 | GLU | A | 330 | −0.872 | −3.365 | 75.697 | 1.00 | 26.90 | O |
| ATOM | 1648 | OE2 | GLU | A | 330 | −3.077 | −3.319 | 75.676 | 1.00 | 24.34 | O |
| ATOM | 1649 | C | GLU | A | 330 | −4.686 | −0.597 | 79.672 | 1.00 | 22.92 | C |
| ATOM | 1650 | O | GLU | A | 330 | −5.180 | −1.461 | 80.393 | 1.00 | 24.06 | O |
| ATOM | 1657 | N | VAL | A | 331 | −5.346 | 0.493 | 79.298 | 1.00 | 22.81 | N |
| ATOM | 1658 | CA | VAL | A | 331 | −6.732 | 0.756 | 79.671 | 1.00 | 22.45 | C |
| ATOM | 1659 | CB | VAL | A | 331 | −6.900 | 2.110 | 80.423 | 1.00 | 22.50 | C |
| ATOM | 1660 | CG1 | VAL | A | 331 | −8.363 | 2.317 | 80.838 | 1.00 | 21.93 | C |
| ATOM | 1661 | CG2 | VAL | A | 331 | −5.957 | 2.191 | 81.626 | 1.00 | 22.88 | C |
| ATOM | 1662 | C | VAL | A | 331 | −7.549 | 0.863 | 78.408 | 1.00 | 21.85 | C |
| ATOM | 1663 | O | VAL | A | 331 | −7.352 | 1.772 | 77.623 | 1.00 | 21.61 | O |
| ATOM | 1673 | N | GLU | A | 332 | −8.495 | −0.038 | 78.238 | 1.00 | 21.24 | N |
| ATOM | 1674 | CA | GLU | A | 332 | −9.425 | 0.048 | 77.132 | 1.00 | 20.97 | C |
| ATOM | 1675 | CB | GLU | A | 332 | −10.418 | −1.104 | 77.176 | 1.00 | 21.12 | C |
| ATOM | 1676 | CG | GLU | A | 332 | −9.776 | −2.477 | 77.121 | 1.00 | 23.19 | C |
| ATOM | 1677 | CD | GLU | A | 332 | −8.993 | −2.703 | 75.848 | 1.00 | 26.02 | C |
| ATOM | 1678 | OE1 | GLU | A | 332 | −7.785 | −3.016 | 75.949 | 1.00 | 28.28 | O |
| ATOM | 1679 | OE2 | GLU | A | 332 | −9.590 | −2.570 | 74.754 | 1.00 | 27.84 | O |
| ATOM | 1680 | C | GLU | A | 332 | −10.187 | 1.348 | 77.165 | 1.00 | 19.96 | C |
| ATOM | 1681 | O | GLU | A | 332 | −10.504 | 1.879 | 78.218 | 1.00 | 19.72 | O |
| ATOM | 1688 | N | LEU | A | 333 | −10.488 | 1.858 | 75.988 | 1.00 | 19.44 | N |
| ATOM | 1689 | CA | LEU | A | 333 | −11.242 | 3.088 | 75.877 | 1.00 | 19.28 | C |
| ATOM | 1690 | CB | LEU | A | 333 | −11.165 | 3.638 | 74.453 | 1.00 | 19.01 | C |
| ATOM | 1691 | CG | LEU | A | 333 | −10.055 | 4.664 | 74.200 | 1.00 | 20.67 | C |
| ATOM | 1692 | CD1 | LEU | A | 333 | −10.074 | 5.756 | 75.269 | 1.00 | 22.16 | C |
| ATOM | 1693 | CD2 | LEU | A | 333 | −8.680 | 4.026 | 74.125 | 1.00 | 20.93 | C |
| ATOM | 1694 | C | LEU | A | 333 | −12.695 | 2.904 | 76.317 | 1.00 | 18.82 | C |
| ATOM | 1695 | O | LEU | A | 333 | −13.313 | 3.853 | 76.766 | 1.00 | 18.43 | O |
| ATOM | 1707 | N | THR | A | 334 | −13.237 | 1.694 | 76.170 | 1.00 | 18.80 | N |
| ATOM | 1708 | CA | THR | A | 334 | −14.569 | 1.396 | 76.689 | 1.00 | 18.74 | C |
| ATOM | 1709 | CB | THR | A | 334 | −15.049 | −0.030 | 76.296 | 1.00 | 18.98 | C |
| ATOM | 1710 | OG1 | THR | A | 334 | −14.020 | −0.987 | 76.558 | 1.00 | 19.88 | O |
| ATOM | 1711 | CG2 | THR | A | 334 | −15.301 | −0.143 | 74.801 | 1.00 | 19.02 | C |
| ATOM | 1712 | C | THR | A | 334 | −14.612 | 1.548 | 78.209 | 1.00 | 18.22 | C |
| ATOM | 1713 | O | THR | A | 334 | −15.624 | 1.962 | 78.754 | 1.00 | 18.16 | O |
| ATOM | 1721 | N | THR | A | 335 | −13.516 | 1.213 | 78.879 | 1.00 | 17.54 | N |
| ATOM | 1722 | CA | THR | A | 335 | −13.408 | 1.401 | 80.318 | 1.00 | 17.39 | C |
| ATOM | 1723 | CB | THR | A | 335 | −12.059 | 0.856 | 80.811 | 1.00 | 17.47 | C |
| ATOM | 1724 | OG1 | THR | A | 335 | −12.048 | −0.569 | 80.708 | 1.00 | 16.53 | O |
| ATOM | 1725 | CG2 | THR | A | 335 | −11.839 | 1.137 | 82.292 | 1.00 | 17.84 | C |
| ATOM | 1726 | C | THR | A | 335 | −13.553 | 2.861 | 80.728 | 1.00 | 17.36 | C |
| ATOM | 1727 | O | THR | A | 335 | −14.264 | 3.176 | 81.680 | 1.00 | 16.74 | O |
| ATOM | 1735 | N | VAL | A | 336 | −12.868 | 3.751 | 80.010 | 1.00 | 17.95 | N |
| ATOM | 1736 | CA | VAL | A | 336 | −12.927 | 5.176 | 80.333 | 1.00 | 17.80 | C |
| ATOM | 1737 | CB | VAL | A | 336 | −11.632 | 6.009 | 79.835 | 1.00 | 18.15 | C |
| ATOM | 1738 | CG1 | VAL | A | 336 | −10.501 | 5.119 | 79.332 | 1.00 | 17.46 | C |
| ATOM | 1739 | CG2 | VAL | A | 336 | −11.965 | 7.078 | 78.851 | 1.00 | 18.06 | C |
| ATOM | 1740 | C | VAL | A | 336 | −14.310 | 5.755 | 79.937 | 1.00 | 17.85 | C |
| ATOM | 1741 | O | VAL | A | 336 | −14.849 | 6.636 | 80.597 | 1.00 | 16.84 | O |
| ATOM | 1751 | N | ALA | A | 337 | −14.924 | 5.198 | 78.903 | 1.00 | 18.24 | N |
| ATOM | 1752 | CA | ALA | A | 337 | −16.296 | 5.563 | 78.594 | 1.00 | 18.38 | C |
| ATOM | 1753 | CB | ALA | A | 337 | −16.741 | 4.939 | 77.282 | 1.00 | 18.32 | C |
| ATOM | 1754 | C | ALA | A | 337 | −17.254 | 5.215 | 79.737 | 1.00 | 18.28 | C |
| ATOM | 1755 | O | ALA | A | 337 | −18.167 | 5.989 | 80.006 | 1.00 | 18.22 | O |
| ATOM | 1761 | N | THR | A | 338 | −17.039 | 4.090 | 80.434 | 1.00 | 18.47 | N |
| ATOM | 1762 | CA | THR | A | 338 | −17.950 | 3.708 | 81.530 | 1.00 | 18.54 | C |
| ATOM | 1763 | CB | THR | A | 338 | −18.113 | 2.124 | 81.746 | 1.00 | 18.74 | C |
| ATOM | 1764 | OG1 | THR | A | 338 | −17.141 | 1.589 | 82.648 | 1.00 | 20.83 | O |
| ATOM | 1765 | CG2 | THR | A | 338 | −17.884 | 1.348 | 80.507 | 1.00 | 18.10 | C |
| ATOM | 1766 | C | THR | A | 338 | −17.695 | 4.442 | 82.860 | 1.00 | 18.24 | C |
| ATOM | 1767 | O | THR | A | 338 | −18.647 | 4.712 | 83.591 | 1.00 | 17.57 | O |
| ATOM | 1775 | N | GLN | A | 339 | −16.451 | 4.823 | 83.130 | 1.00 | 18.24 | N |
| ATOM | 1776 | CA | GLN | A | 339 | −16.070 | 5.392 | 84.426 | 1.00 | 19.20 | C |
| ATOM | 1777 | CB | GLN | A | 339 | −14.820 | 4.684 | 84.943 | 1.00 | 19.05 | C |
| ATOM | 1778 | CG | GLN | A | 339 | −15.078 | 3.240 | 85.322 | 1.00 | 20.19 | C |
| ATOM | 1779 | CD | GLN | A | 339 | −13.879 | 2.590 | 85.945 | 1.00 | 21.10 | C |
| ATOM | 1780 | OE1 | GLN | A | 339 | −13.344 | 3.098 | 86.920 | 1.00 | 22.83 | O |
| ATOM | 1781 | NE2 | GLN | A | 339 | −13.448 | 1.463 | 85.387 | 1.00 | 21.99 | N |
| ATOM | 1782 | C | GLN | A | 339 | −15.840 | 6.907 | 84.489 | 1.00 | 19.73 | C |
| ATOM | 1783 | O | GLN | A | 339 | −16.204 | 7.553 | 85.465 | 1.00 | 19.19 | O |
| ATOM | 1792 | N | ALA | A | 340 | −15.222 | 7.462 | 83.454 | 1.00 | 21.16 | N |
| ATOM | 1793 | CA | ALA | A | 340 | −14.806 | 8.873 | 83.430 | 1.00 | 22.03 | C |
| ATOM | 1794 | CB | ALA | A | 340 | −13.871 | 9.125 | 82.253 | 1.00 | 21.95 | C |
| ATOM | 1795 | C | ALA | A | 340 | −15.988 | 9.821 | 83.347 | 1.00 | 22.76 | C |
| ATOM | 1796 | O | ALA | A | 340 | −16.978 | 9.523 | 82.682 | 1.00 | 23.46 | O |
| ATOM | 1802 | N | GLY | A | 341 | −15.877 | 10.961 | 84.021 | 1.00 | 23.53 | N |
| ATOM | 1803 | CA | GLY | A | 341 | −16.838 | 12.044 | 83.895 | 1.00 | 24.13 | C |
| ATOM | 1804 | C | GLY | A | 341 | −16.775 | 12.754 | 82.550 | 1.00 | 24.34 | C |
| ATOM | 1805 | O | GLY | A | 341 | −16.014 | 12.380 | 81.668 | 1.00 | 24.94 | O |
| ATOM | 1809 | N | SER | A | 342 | −17.570 | 13.806 | 82.399 | 1.00 | 24.92 | N |
| ATOM | 1810 | CA | SER | A | 342 | −17.614 | 14.566 | 81.143 | 1.00 | 25.19 | C |
| ATOM | 1811 | CB | SER | A | 342 | −18.543 | 15.784 | 81.265 | 1.00 | 25.60 | C |
| ATOM | 1812 | OG | SER | A | 342 | −19.575 | 15.568 | 82.227 | 1.00 | 28.38 | O |
| ATOM | 1813 | C | SER | A | 342 | −16.227 | 15.048 | 80.749 | 1.00 | 24.46 | C |
| ATOM | 1814 | O | SER | A | 342 | −15.784 | 14.815 | 79.633 | 1.00 | 24.97 | O |
| ATOM | 1820 | N | LEU | A | 343 | −15.550 | 15.693 | 81.692 | 1.00 | 23.79 | N |
| ATOM | 1821 | CA | LEU | A | 343 | −14.278 | 16.351 | 81.437 | 1.00 | 23.55 | C |
| ATOM | 1822 | CB | LEU | A | 343 | −13.844 | 17.182 | 82.644 | 1.00 | 23.52 | C |
| ATOM | 1823 | CG | LEU | A | 343 | −14.600 | 18.480 | 82.897 | 1.00 | 24.00 | C |
| ATOM | 1824 | CD1 | LEU | A | 343 | −13.956 | 19.204 | 84.067 | 1.00 | 24.78 | C |
| ATOM | 1825 | CD2 | LEU | A | 343 | −14.620 | 19.364 | 81.653 | 1.00 | 25.15 | C |
| ATOM | 1826 | C | LEU | A | 343 | −13.165 | 15.395 | 81.083 | 1.00 | 23.06 | C |
| ATOM | 1827 | O | LEU | A | 343 | −12.433 | 15.616 | 80.118 | 1.00 | 23.38 | O |
| ATOM | 1839 | N | LEU | A | 344 | −13.022 | 14.340 | 81.863 | 1.00 | 22.69 | N |
| ATOM | 1840 | CA | LEU | A | 344 | −11.921 | 13.424 | 81.645 | 1.00 | 22.40 | C |
| ATOM | 1841 | CB | LEU | A | 344 | −11.818 | 12.411 | 82.780 | 1.00 | 22.47 | C |
| ATOM | 1842 | CG | LEU | A | 344 | −10.700 | 11.354 | 82.661 | 1.00 | 22.32 | C |
| ATOM | 1843 | CD1 | LEU | A | 344 | −9.268 | 11.970 | 82.503 | 1.00 | 21.58 | C |
| ATOM | 1844 | CD2 | LEU | A | 344 | −10.756 | 10.389 | 83.825 | 1.00 | 22.06 | C |
| ATOM | 1845 | C | LEU | A | 344 | −12.115 | 12.725 | 80.322 | 1.00 | 22.77 | C |
| ATOM | 1846 | O | LEU | A | 344 | −11.164 | 12.542 | 79.571 | 1.00 | 22.48 | O |
| ATOM | 1858 | N | HIS | A | 345 | −13.356 | 12.351 | 80.026 | 1.00 | 23.19 | N |
| ATOM | 1859 | CA | HIS | A | 345 | −13.638 | 11.614 | 78.807 | 1.00 | 23.36 | C |
| ATOM | 1860 | CB | HIS | A | 345 | −15.097 | 11.119 | 78.790 | 1.00 | 23.31 | C |
| ATOM | 1861 | CG | HIS | A | 345 | −15.318 | 9.950 | 77.878 | 1.00 | 22.34 | C |
| ATOM | 1862 | ND1 | HIS | A | 345 | −16.559 | 9.603 | 77.401 | 1.00 | 22.38 | N |
| ATOM | 1863 | CE1 | HIS | A | 345 | −16.444 | 8.552 | 76.609 | 1.00 | 22.26 | C |
| ATOM | 1864 | NE2 | HIS | A | 345 | −15.170 | 8.212 | 76.549 | 1.00 | 21.16 | N |
| ATOM | 1865 | CD2 | HIS | A | 345 | −14.447 | 9.066 | 77.339 | 1.00 | 21.74 | C |
| ATOM | 1866 | C | HIS | A | 345 | −13.305 | 12.438 | 77.557 | 1.00 | 23.50 | C |
| ATOM | 1867 | O | HIS | A | 345 | −12.677 | 11.938 | 76.627 | 1.00 | 23.36 | O |
| ATOM | 1876 | N | SER | A | 346 | −13.694 | 13.713 | 77.559 | 1.00 | 24.22 | N |
| ATOM | 1877 | CA | SER | A | 346 | −13.375 | 14.645 | 76.461 | 1.00 | 24.23 | C |
| ATOM | 1878 | CB | SER | A | 346 | −13.820 | 16.089 | 76.751 | 1.00 | 24.19 | C |
| ATOM | 1879 | OG | SER | A | 346 | −15.085 | 16.161 | 77.385 | 1.00 | 26.68 | O |
| ATOM | 1880 | C | SER | A | 346 | −11.896 | 14.693 | 76.240 | 1.00 | 23.63 | C |
| ATOM | 1881 | O | SER | A | 346 | −11.427 | 14.631 | 75.118 | 1.00 | 24.30 | O |
| ATOM | 1887 | N | LEU | A | 347 | −11.175 | 14.799 | 77.340 | 1.00 | 23.56 | N |
| ATOM | 1888 | CA | LEU | A | 347 | −9.743 | 15.024 | 77.324 | 1.00 | 23.54 | C |
| ATOM | 1889 | CB | LEU | A | 347 | −9.236 | 15.149 | 78.747 | 1.00 | 23.76 | C |
| ATOM | 1890 | CG | LEU | A | 347 | −8.484 | 16.408 | 79.117 | 1.00 | 23.71 | C |
| ATOM | 1891 | CD1 | LEU | A | 347 | −7.814 | 16.164 | 80.452 | 1.00 | 23.50 | C |
| ATOM | 1892 | CD2 | LEU | A | 347 | −7.482 | 16.795 | 78.038 | 1.00 | 25.09 | C |
| ATOM | 1893 | C | LEU | A | 347 | −9.018 | 13.882 | 76.665 | 1.00 | 23.32 | C |
| ATOM | 1894 | O | LEU | A | 347 | −8.153 | 14.074 | 75.824 | 1.00 | 23.62 | O |
| ATOM | 1906 | N | VAL | A | 348 | −9.387 | 12.688 | 77.096 | 1.00 | 23.36 | N |
| ATOM | 1907 | CA | VAL | A | 348 | −8.800 | 11.445 | 76.630 | 1.00 | 23.16 | C |
| ATOM | 1908 | CB | VAL | A | 348 | −9.413 | 10.240 | 77.391 | 1.00 | 23.13 | C |
| ATOM | 1909 | CG1 | VAL | A | 348 | −9.129 | 8.883 | 76.682 | 1.00 | 22.86 | C |
| ATOM | 1910 | CG2 | VAL | A | 348 | −8.898 | 10.227 | 78.829 | 1.00 | 23.03 | C |
| ATOM | 1911 | C | VAL | A | 348 | −9.014 | 11.269 | 75.141 | 1.00 | 22.68 | C |
| ATOM | 1912 | O | VAL | A | 348 | −8.082 | 10.883 | 74.416 | 1.00 | 22.01 | O |
| ATOM | 1922 | N | LEU | A | 349 | −10.239 | 11.551 | 74.713 | 1.00 | 22.18 | N |
| ATOM | 1923 | CA | LEU | A | 349 | −10.636 | 11.365 | 73.325 | 1.00 | 22.29 | C |
| ATOM | 1924 | CB | LEU | A | 349 | −12.169 | 11.505 | 73.202 | 1.00 | 22.40 | C |
| ATOM | 1925 | CG | LEU | A | 349 | −13.042 | 10.231 | 73.140 | 1.00 | 22.26 | C |
| ATOM | 1926 | CD1 | LEU | A | 349 | −12.383 | 9.018 | 73.745 | 1.00 | 22.59 | C |
| ATOM | 1927 | CD2 | LEU | A | 349 | −14.412 | 10.459 | 73.771 | 1.00 | 21.68 | C |
| ATOM | 1928 | C | LEU | A | 349 | −9.914 | 12.355 | 72.408 | 1.00 | 21.94 | C |
| ATOM | 1929 | O | LEU | A | 349 | −9.426 | 11.988 | 71.340 | 1.00 | 20.92 | O |
| ATOM | 1941 | N | ARG | A | 350 | −9.831 | 13.605 | 72.864 | 1.00 | 22.19 | N |
| ATOM | 1942 | CA | ARG | A | 350 | −9.199 | 14.656 | 72.106 | 1.00 | 22.13 | C |
| ATOM | 1943 | CB | ARG | A | 350 | −9.381 | 16.009 | 72.778 | 1.00 | 22.59 | C |
| ATOM | 1944 | CG | ARG | A | 350 | −9.178 | 17.203 | 71.837 | 1.00 | 24.07 | C |
| ATOM | 1945 | CD | ARG | A | 350 | −9.318 | 18.616 | 72.504 | 1.00 | 27.29 | C |
| ATOM | 1946 | NE | ARG | A | 350 | −8.912 | 19.683 | 71.578 | 1.00 | 29.98 | N |
| ATOM | 1947 | CZ | ARG | A | 350 | −8.714 | 20.966 | 71.887 | 1.00 | 29.24 | C |
| ATOM | 1948 | NH1 | ARG | A | 350 | −8.890 | 21.409 | 73.109 | 1.00 | 31.36 | N |
| ATOM | 1949 | NH2 | ARG | A | 350 | −8.356 | 21.820 | 70.943 | 1.00 | 28.95 | N |
| ATOM | 1950 | C | ARG | A | 350 | −7.750 | 14.313 | 71.967 | 1.00 | 22.02 | C |
| ATOM | 1951 | O | ARG | A | 350 | −7.182 | 14.474 | 70.901 | 1.00 | 22.49 | O |
| ATOM | 1965 | N | ALA | A | 351 | −7.150 | 13.812 | 73.039 | 1.00 | 22.06 | N |
| ATOM | 1966 | CA | ALA | A | 351 | −5.769 | 13.341 | 72.994 | 1.00 | 21.85 | C |
| ATOM | 1967 | CB | ALA | A | 351 | −5.328 | 12.896 | 74.380 | 1.00 | 21.83 | C |
| ATOM | 1968 | C | ALA | A | 351 | −5.547 | 12.227 | 71.960 | 1.00 | 22.06 | C |
| ATOM | 1969 | O | ALA | A | 351 | −4.539 | 12.221 | 71.240 | 1.00 | 22.56 | O |
| ATOM | 1975 | N | GLN | A | 352 | −6.493 | 11.307 | 71.849 | 1.00 | 22.06 | N |
| ATOM | 1976 | CA | GLN | A | 352 | −6.337 | 10.190 | 70.924 | 1.00 | 22.40 | C |
| ATOM | 1977 | CB | GLN | A | 352 | −7.387 | 9.090 | 71.181 | 1.00 | 22.43 | C |
| ATOM | 1978 | CG | GLN | A | 352 | −7.176 | 8.293 | 72.477 | 1.00 | 21.35 | C |
| ATOM | 1979 | CD | GLN | A | 352 | −5.899 | 7.507 | 72.470 | 1.00 | 19.06 | C |
| ATOM | 1980 | OE1 | GLN | A | 352 | −4.886 | 8.005 | 73.158 | 1.00 | 20.41 | O |
| ATOM | 1981 | NE2 | GLN | A | 352 | −5.824 | 6.467 | 71.841 | 1.00 | 18.10 | N |
| ATOM | 1982 | C | GLN | A | 352 | −6.398 | 10.639 | 69.483 | 1.00 | 22.76 | C |
| ATOM | 1983 | O | GLN | A | 352 | −5.761 | 10.033 | 68.623 | 1.00 | 23.61 | O |
| ATOM | 1992 | N | GLU | A | 353 | −7.159 | 11.696 | 69.220 | 1.00 | 23.08 | N |
| ATOM | 1993 | CA | GLU | A | 353 | −7.217 | 12.292 | 67.887 | 1.00 | 23.32 | C |
| ATOM | 1994 | CB | GLU | A | 353 | −8.176 | 13.449 | 67.885 | 1.00 | 23.35 | C |
| ATOM | 1995 | CG | GLU | A | 353 | −9.584 | 13.049 | 68.224 | 1.00 | 24.44 | C |
| ATOM | 1996 | CD | GLU | A | 353 | −10.539 | 14.154 | 67.904 | 1.00 | 26.39 | C |
| ATOM | 1997 | OE1 | GLU | A | 353 | −10.961 | 14.890 | 68.825 | 1.00 | 28.35 | O |
| ATOM | 1998 | OE2 | GLU | A | 353 | −10.831 | 14.292 | 66.708 | 1.00 | 29.40 | O |
| ATOM | 1999 | C | GLU | A | 353 | −5.866 | 12.818 | 67.458 | 1.00 | 23.60 | C |
| ATOM | 2000 | O | GLU | A | 353 | −5.440 | 12.638 | 66.315 | 1.00 | 24.20 | O |
| ATOM | 2007 | N | LEU | A | 354 | −5.204 | 13.467 | 68.399 | 1.00 | 23.71 | N |
| ATOM | 2008 | CA | LEU | A | 354 | −3.849 | 13.932 | 68.228 | 1.00 | 24.09 | C |
| ATOM | 2009 | CB | LEU | A | 354 | −3.464 | 14.800 | 69.430 | 1.00 | 24.13 | C |
| ATOM | 2010 | CG | LEU | A | 354 | −2.112 | 15.477 | 69.243 | 1.00 | 26.09 | C |
| ATOM | 2011 | CD1 | LEU | A | 354 | −2.152 | 16.359 | 67.957 | 1.00 | 28.57 | C |
| ATOM | 2012 | CD2 | LEU | A | 354 | −1.651 | 16.269 | 70.454 | 1.00 | 26.57 | C |
| ATOM | 2013 | C | LEU | A | 354 | −2.839 | 12.795 | 68.040 | 1.00 | 23.91 | C |
| ATOM | 2014 | O | LEU | A | 354 | −1.865 | 12.935 | 67.306 | 1.00 | 24.26 | O |
| ATOM | 2026 | N | VAL | A | 355 | −3.066 | 11.667 | 68.698 | 1.00 | 24.35 | N |
| ATOM | 2027 | CA | VAL | A | 355 | −2.215 | 10.493 | 68.503 | 1.00 | 24.18 | C |
| ATOM | 2028 | CB | VAL | A | 355 | −2.624 | 9.365 | 69.489 | 1.00 | 24.01 | C |
| ATOM | 2029 | CG1 | VAL | A | 355 | −1.894 | 8.060 | 69.196 | 1.00 | 24.42 | C |
| ATOM | 2030 | CG2 | VAL | A | 355 | −2.347 | 9.769 | 70.892 | 1.00 | 24.75 | C |
| ATOM | 2031 | C | VAL | A | 355 | −2.317 | 10.005 | 67.024 | 1.00 | 24.31 | C |
| ATOM | 2032 | O | VAL | A | 355 | −1.323 | 9.634 | 66.370 | 1.00 | 23.40 | O |
| ATOM | 2042 | N | LEU | A | 356 | −3.544 | 10.040 | 66.524 | 1.00 | 24.17 | N |
| ATOM | 2043 | CA | LEU | A | 356 | −3.874 | 9.570 | 65.213 | 1.00 | 24.28 | C |
| ATOM | 2044 | CB | LEU | A | 356 | −5.395 | 9.606 | 65.054 | 1.00 | 24.58 | C |
| ATOM | 2045 | CG | LEU | A | 356 | −6.088 | 8.520 | 64.232 | 1.00 | 24.94 | C |
| ATOM | 2046 | CD1 | LEU | A | 356 | −5.483 | 7.125 | 64.437 | 1.00 | 25.65 | C |
| ATOM | 2047 | CD2 | LEU | A | 356 | −7.579 | 8.518 | 64.562 | 1.00 | 24.91 | C |
| ATOM | 2048 | C | LEU | A | 356 | −3.203 | 10.447 | 64.169 | 1.00 | 24.53 | C |
| ATOM | 2049 | O | LEU | A | 356 | −2.713 | 9.953 | 63.138 | 1.00 | 24.41 | O |
| ATOM | 2061 | N | GLN | A | 357 | −3.179 | 11.745 | 64.445 | 1.00 | 24.33 | N |
| ATOM | 2062 | CA | GLN | A | 357 | −2.534 | 12.727 | 63.579 | 1.00 | 24.45 | C |
| ATOM | 2063 | CB | GLN | A | 357 | −2.777 | 14.125 | 64.138 | 1.00 | 25.09 | C |
| ATOM | 2064 | CG | GLN | A | 357 | −4.066 | 14.750 | 63.666 | 1.00 | 28.14 | C |
| ATOM | 2065 | CD | GLN | A | 357 | −3.798 | 15.997 | 62.851 | 1.00 | 32.13 | C |
| ATOM | 2066 | OE1 | GLN | A | 357 | −4.095 | 15.955 | 61.539 | 1.00 | 35.00 | O |
| ATOM | 2067 | NE2 | GLN | A | 357 | −3.299 | 16.981 | 63.399 | 1.00 | 32.18 | N |
| ATOM | 2068 | C | GLN | A | 357 | −1.032 | 12.517 | 63.464 | 1.00 | 23.60 | C |
| ATOM | 2069 | O | GLN | A | 357 | −0.473 | 12.590 | 62.375 | 1.00 | 23.35 | O |
| ATOM | 2078 | N | LEU | A | 358 | −0.390 | 12.266 | 64.600 | 1.00 | 22.82 | N |
| ATOM | 2079 | CA | LEU | A | 358 | 1.049 | 12.092 | 64.640 | 1.00 | 22.51 | C |
| ATOM | 2080 | CB | LEU | A | 358 | 1.562 | 12.179 | 66.069 | 1.00 | 22.81 | C |
| ATOM | 2081 | CG | LEU | A | 358 | 1.443 | 13.586 | 66.659 | 1.00 | 23.01 | C |
| ATOM | 2082 | CD1 | LEU | A | 358 | 1.601 | 13.593 | 68.160 | 1.00 | 24.29 | C |
| ATOM | 2083 | CD2 | LEU | A | 358 | 2.473 | 14.479 | 66.052 | 1.00 | 23.76 | C |
| ATOM | 2084 | C | LEU | A | 358 | 1.455 | 10.779 | 64.019 | 1.00 | 22.50 | C |
| ATOM | 2085 | O | LEU | A | 358 | 2.538 | 10.670 | 63.465 | 1.00 | 22.59 | O |
| ATOM | 2097 | N | LEU | A | 359 | 0.575 | 9.790 | 64.088 | 1.00 | 22.25 | N |
| ATOM | 2098 | CA | LEU | A | 359 | 0.796 | 8.521 | 63.407 | 1.00 | 22.01 | C |
| ATOM | 2099 | CB | LEU | A | 359 | −0.194 | 7.446 | 63.906 | 1.00 | 22.06 | C |
| ATOM | 2100 | CG | LEU | A | 359 | 0.156 | 6.779 | 65.246 | 1.00 | 21.59 | C |
| ATOM | 2101 | CD1 | LEU | A | 359 | −1.011 | 5.953 | 65.726 | 1.00 | 21.97 | C |
| ATOM | 2102 | CD2 | LEU | A | 359 | 1.396 | 5.896 | 65.134 | 1.00 | 21.34 | C |
| ATOM | 2103 | C | LEU | A | 359 | 0.694 | 8.680 | 61.890 | 1.00 | 21.75 | C |
| ATOM | 2104 | O | LEU | A | 359 | 1.442 | 8.044 | 61.162 | 1.00 | 20.95 | O |
| ATOM | 2116 | N | ALA | A | 360 | −0.214 | 9.535 | 61.424 | 1.00 | 21.71 | N |
| ATOM | 2117 | CA | ALA | A | 360 | −0.355 | 9.785 | 59.996 | 1.00 | 21.95 | C |
| ATOM | 2118 | CB | ALA | A | 360 | −1.653 | 10.489 | 59.709 | 1.00 | 21.96 | C |
| ATOM | 2119 | C | ALA | A | 360 | 0.833 | 10.588 | 59.449 | 1.00 | 22.18 | C |
| ATOM | 2120 | O | ALA | A | 360 | 1.124 | 10.537 | 58.255 | 1.00 | 23.10 | O |
| ATOM | 2126 | N | LEU | A | 361 | 1.532 | 11.304 | 60.323 | 1.00 | 21.79 | N |
| ATOM | 2127 | CA | LEU | A | 361 | 2.734 | 12.029 | 59.953 | 1.00 | 21.59 | C |
| ATOM | 2128 | CB | LEU | A | 361 | 2.882 | 13.282 | 60.814 | 1.00 | 21.35 | C |
| ATOM | 2129 | CG | LEU | A | 361 | 1.787 | 14.312 | 60.582 | 1.00 | 21.58 | C |
| ATOM | 2130 | CD1 | LEU | A | 361 | 1.823 | 15.349 | 61.641 | 1.00 | 22.85 | C |
| ATOM | 2131 | CD2 | LEU | A | 361 | 1.929 | 14.940 | 59.210 | 1.00 | 23.29 | C |
| ATOM | 2132 | C | LEU | A | 361 | 3.970 | 11.168 | 60.124 | 1.00 | 21.89 | C |
| ATOM | 2133 | O | LEU | A | 361 | 5.082 | 11.632 | 59.888 | 1.00 | 22.06 | O |
| ATOM | 2145 | N | GLN | A | 362 | 3.780 | 9.925 | 60.548 | 1.00 | 22.19 | N |
| ATOM | 2146 | CA | GLN | A | 362 | 4.872 | 8.978 | 60.751 | 1.00 | 22.51 | C |
| ATOM | 2147 | CB | GLN | A | 362 | 5.539 | 8.613 | 59.421 | 1.00 | 22.99 | C |
| ATOM | 2148 | CG | GLN | A | 362 | 4.593 | 8.134 | 58.311 | 1.00 | 24.14 | C |
| ATOM | 2149 | CD | GLN | A | 362 | 5.357 | 7.747 | 57.061 | 1.00 | 26.67 | C |
| ATOM | 2150 | OE1 | GLN | A | 362 | 5.269 | 8.416 | 56.020 | 1.00 | 29.67 | O |
| ATOM | 2151 | NE2 | GLN | A | 362 | 6.124 | 6.670 | 57.158 | 1.00 | 28.21 | N |
| ATOM | 2152 | C | GLN | A | 362 | 5.904 | 9.522 | 61.725 | 1.00 | 22.18 | C |
| ATOM | 2153 | O | GLN | A | 362 | 7.114 | 9.498 | 61.463 | 1.00 | 22.36 | O |
| ATOM | 2162 | N | LEU | A | 363 | 5.402 | 10.024 | 62.845 | 1.00 | 22.16 | N |
| ATOM | 2163 | CA | LEU | A | 363 | 6.215 | 10.644 | 63.899 | 1.00 | 22.18 | C |
| ATOM | 2164 | CB | LEU | A | 363 | 5.309 | 11.051 | 65.061 | 1.00 | 22.05 | C |
| ATOM | 2165 | CG | LEU | A | 363 | 5.640 | 12.107 | 66.122 | 1.00 | 22.81 | C |
| ATOM | 2166 | CD1 | LEU | A | 363 | 5.555 | 11.496 | 67.535 | 1.00 | 22.62 | C |
| ATOM | 2167 | CD2 | LEU | A | 363 | 6.955 | 12.822 | 65.926 | 1.00 | 23.42 | C |
| ATOM | 2168 | C | LEU | A | 363 | 7.243 | 9.631 | 64.371 | 1.00 | 22.00 | C |
| ATOM | 2169 | O | LEU | A | 363 | 6.896 | 8.475 | 64.607 | 1.00 | 22.21 | O |
| ATOM | 2181 | N | ASP | A | 364 | 8.503 | 10.042 | 64.478 | 1.00 | 21.65 | N |
| ATOM | 2182 | CA | ASP | A | 364 | 9.537 | 9.149 | 64.985 | 1.00 | 21.27 | C |
| ATOM | 2183 | CB | ASP | A | 364 | 10.499 | 8.712 | 63.858 | 1.00 | 21.21 | C |
| ATOM | 2184 | CG | ASP | A | 364 | 11.354 | 9.836 | 63.320 | 1.00 | 21.38 | C |
| ATOM | 2185 | OD1 | ASP | A | 364 | 11.606 | 10.791 | 64.059 | 1.00 | 22.38 | O |
| ATOM | 2186 | OD2 | ASP | A | 364 | 11.837 | 9.845 | 62.161 | 1.00 | 22.50 | O |
| ATOM | 2187 | C | ASP | A | 364 | 10.247 | 9.721 | 66.229 | 1.00 | 21.01 | C |
| ATOM | 2188 | O | ASP | A | 364 | 9.990 | 10.846 | 66.644 | 1.00 | 20.30 | O |
| ATOM | 2193 | N | ARG | A | 365 | 11.105 | 8.907 | 66.830 | 1.00 | 21.05 | N |
| ATOM | 2194 | CA | ARG | A | 365 | 11.795 | 9.229 | 68.078 | 1.00 | 21.57 | C |
| ATOM | 2195 | CB | ARG | A | 365 | 12.621 | 8.013 | 68.524 | 1.00 | 22.05 | C |
| ATOM | 2196 | CG | ARG | A | 365 | 13.409 | 8.152 | 69.843 | 1.00 | 24.28 | C |
| ATOM | 2197 | CD | ARG | A | 365 | 14.001 | 6.793 | 70.358 | 1.00 | 26.84 | C |
| ATOM | 2198 | NE | ARG | A | 365 | 14.229 | 6.735 | 71.813 | 1.00 | 29.63 | N |
| ATOM | 2199 | CZ | ARG | A | 365 | 13.268 | 6.761 | 72.761 | 1.00 | 31.96 | C |
| ATOM | 2200 | NH1 | ARG | A | 365 | 11.974 | 6.858 | 72.443 | 1.00 | 33.32 | N |
| ATOM | 2201 | NH2 | ARG | A | 365 | 13.601 | 6.699 | 74.051 | 1.00 | 32.82 | N |
| ATOM | 2202 | C | ARG | A | 365 | 12.704 | 10.454 | 67.945 | 1.00 | 21.29 | C |
| ATOM | 2203 | O | ARG | A | 365 | 12.928 | 11.177 | 68.908 | 1.00 | 20.92 | O |
| ATOM | 2217 | N | GLN | A | 366 | 13.237 | 10.668 | 66.745 | 1.00 | 20.91 | N |
| ATOM | 2218 | CA | GLN | A | 366 | 14.111 | 11.806 | 66.479 | 1.00 | 20.19 | C |
| ATOM | 2219 | CB | GLN | A | 366 | 14.733 | 11.705 | 65.083 | 1.00 | 20.55 | C |
| ATOM | 2220 | CG | GLN | A | 366 | 15.916 | 10.766 | 64.976 | 1.00 | 20.66 | C |
| ATOM | 2221 | CD | GLN | A | 366 | 15.560 | 9.303 | 65.001 | 1.00 | 21.12 | C |
| ATOM | 2222 | OE1 | GLN | A | 366 | 14.295 | 8.973 | 64.793 | 1.00 | 25.33 | O |
| ATOM | 2223 | NE2 | GLN | A | 366 | 16.446 | 8.472 | 65.184 | 1.00 | 20.14 | N |
| ATOM | 2224 | C | GLN | A | 366 | 13.304 | 13.077 | 66.554 | 1.00 | 19.24 | C |
| ATOM | 2225 | O | GLN | A | 366 | 13.751 | 14.082 | 67.091 | 1.00 | 18.56 | O |
| ATOM | 2234 | N | GLU | A | 367 | 12.118 | 13.017 | 65.978 | 1.00 | 18.48 | N |
| ATOM | 2235 | CA | GLU | A | 367 | 11.237 | 14.143 | 65.960 | 1.00 | 19.03 | C |
| ATOM | 2236 | CB | GLU | A | 367 | 10.118 | 13.897 | 64.969 | 1.00 | 19.13 | C |
| ATOM | 2237 | CG | GLU | A | 367 | 10.588 | 14.102 | 63.532 | 1.00 | 20.28 | C |
| ATOM | 2238 | CD | GLU | A | 367 | 9.763 | 13.378 | 62.492 | 1.00 | 19.78 | C |
| ATOM | 2239 | OE1 | GLU | A | 367 | 8.766 | 12.740 | 62.851 | 1.00 | 21.59 | O |
| ATOM | 2240 | OE2 | GLU | A | 367 | 10.115 | 13.458 | 61.303 | 1.00 | 22.21 | O |
| ATOM | 2241 | C | GLU | A | 367 | 10.710 | 14.356 | 67.345 | 1.00 | 19.37 | C |
| ATOM | 2242 | O | GLU | A | 367 | 10.595 | 15.481 | 67.815 | 1.00 | 19.16 | O |
| ATOM | 2249 | N | PHE | A | 368 | 10.452 | 13.251 | 68.026 | 1.00 | 20.19 | N |
| ATOM | 2250 | CA | PHE | A | 368 | 9.898 | 13.289 | 69.370 | 1.00 | 20.38 | C |
| ATOM | 2251 | CB | PHE | A | 368 | 9.657 | 11.869 | 69.887 | 1.00 | 20.68 | C |
| ATOM | 2252 | CG | PHE | A | 368 | 9.538 | 11.769 | 71.377 | 1.00 | 20.49 | C |
| ATOM | 2253 | CD1 | PHE | A | 368 | 8.445 | 12.294 | 72.033 | 1.00 | 20.62 | C |
| ATOM | 2254 | CE1 | PHE | A | 368 | 8.323 | 12.181 | 73.421 | 1.00 | 21.22 | C |
| ATOM | 2255 | CZ | PHE | A | 368 | 9.303 | 11.527 | 74.143 | 1.00 | 21.40 | C |
| ATOM | 2256 | CE2 | PHE | A | 368 | 10.395 | 10.994 | 73.497 | 1.00 | 21.17 | C |
| ATOM | 2257 | CD2 | PHE | A | 368 | 10.513 | 11.109 | 72.117 | 1.00 | 21.52 | C |
| ATOM | 2258 | C | PHE | A | 368 | 10.805 | 14.075 | 70.293 | 1.00 | 19.99 | C |
| ATOM | 2259 | O | PHE | A | 368 | 10.362 | 15.023 | 70.895 | 1.00 | 21.04 | O |
| ATOM | 2269 | N | VAL | A | 369 | 12.075 | 13.729 | 70.378 | 1.00 | 19.92 | N |
| ATOM | 2270 | CA | VAL | A | 369 | 12.944 | 14.425 | 71.318 | 1.00 | 20.08 | C |
| ATOM | 2271 | CB | VAL | A | 369 | 14.292 | 13.700 | 71.587 | 1.00 | 20.31 | C |
| ATOM | 2272 | CG1 | VAL | A | 369 | 14.045 | 12.261 | 72.035 | 1.00 | 20.80 | C |
| ATOM | 2273 | CG2 | VAL | A | 369 | 15.241 | 13.745 | 70.381 | 1.00 | 21.33 | C |
| ATOM | 2274 | C | VAL | A | 369 | 13.197 | 15.869 | 70.930 | 1.00 | 20.34 | C |
| ATOM | 2275 | O | VAL | A | 369 | 13.441 | 16.685 | 71.808 | 1.00 | 20.38 | O |
| ATOM | 2285 | N | CYS | A | 370 | 13.148 | 16.187 | 69.627 | 1.00 | 20.31 | N |
| ATOM | 2286 | CA | CYS | A | 370 | 13.322 | 17.558 | 69.166 | 1.00 | 19.84 | C |
| ATOM | 2287 | CB | CYS | A | 370 | 13.453 | 17.605 | 67.646 | 1.00 | 19.99 | C |
| ATOM | 2288 | SG | CYS | A | 370 | 14.158 | 19.145 | 67.022 | 1.00 | 19.10 | S |
| ATOM | 2289 | C | CYS | A | 370 | 12.146 | 18.407 | 69.594 | 1.00 | 19.85 | C |
| ATOM | 2290 | O | CYS | A | 370 | 12.282 | 19.551 | 70.009 | 1.00 | 19.53 | O |
| ATOM | 2296 | N | LEU | A | 371 | 10.977 | 17.823 | 69.468 | 1.00 | 20.22 | N |
| ATOM | 2297 | CA | LEU | A | 371 | 9.740 | 18.509 | 69.760 | 1.00 | 20.66 | C |
| ATOM | 2298 | CB | LEU | A | 371 | 8.577 | 17.642 | 69.276 | 1.00 | 20.45 | C |
| ATOM | 2299 | CG | LEU | A | 371 | 7.778 | 17.912 | 67.995 | 1.00 | 20.58 | C |
| ATOM | 2300 | CD1 | LEU | A | 371 | 8.258 | 19.027 | 67.150 | 1.00 | 21.11 | C |
| ATOM | 2301 | CD2 | LEU | A | 371 | 7.627 | 16.631 | 67.175 | 1.00 | 20.00 | C |
| ATOM | 2302 | C | LEU | A | 371 | 9.619 | 18.783 | 71.278 | 1.00 | 20.85 | C |
| ATOM | 2303 | O | LEU | A | 371 | 9.131 | 19.825 | 71.695 | 1.00 | 20.85 | O |
| ATOM | 2315 | N | LYS | A | 372 | 10.074 | 17.856 | 72.109 | 1.00 | 20.71 | N |
| ATOM | 2316 | CA | LYS | A | 372 | 10.015 | 18.089 | 73.527 | 1.00 | 20.77 | C |
| ATOM | 2317 | CB | LYS | A | 372 | 9.928 | 16.775 | 74.298 | 1.00 | 21.39 | C |
| ATOM | 2318 | CG | LYS | A | 372 | 11.137 | 15.961 | 74.569 | 1.00 | 24.12 | C |
| ATOM | 2319 | CD | LYS | A | 372 | 10.653 | 14.549 | 75.043 | 1.00 | 25.22 | C |
| ATOM | 2320 | CE | LYS | A | 372 | 11.720 | 13.715 | 75.709 | 1.00 | 25.11 | C |
| ATOM | 2321 | NZ | LYS | A | 372 | 11.622 | 13.897 | 77.187 | 1.00 | 27.63 | N |
| ATOM | 2322 | C | LYS | A | 372 | 11.049 | 19.081 | 74.036 | 1.00 | 20.17 | C |
| ATOM | 2323 | O | LYS | A | 372 | 10.791 | 19.780 | 74.990 | 1.00 | 19.82 | O |
| ATOM | 2337 | N | PHE | A | 373 | 12.170 | 19.220 | 73.340 | 1.00 | 19.81 | N |
| ATOM | 2338 | CA | PHE | A | 373 | 13.108 | 20.283 | 73.610 | 1.00 | 18.91 | C |
| ATOM | 2339 | CB | PHE | A | 373 | 14.414 | 19.991 | 72.858 | 1.00 | 19.08 | C |
| ATOM | 2340 | CG | PHE | A | 373 | 15.546 | 20.973 | 73.141 | 1.00 | 19.84 | C |
| ATOM | 2341 | CD1 | PHE | A | 373 | 16.382 | 20.808 | 74.240 | 1.00 | 20.35 | C |
| ATOM | 2342 | CE1 | PHE | A | 373 | 17.418 | 21.707 | 74.491 | 1.00 | 20.35 | C |
| ATOM | 2343 | CZ | PHE | A | 373 | 17.625 | 22.778 | 73.646 | 1.00 | 19.59 | C |
| ATOM | 2344 | CE2 | PHE | A | 373 | 16.802 | 22.959 | 72.547 | 1.00 | 20.08 | C |
| ATOM | 2345 | CD2 | PHE | A | 373 | 15.776 | 22.057 | 72.298 | 1.00 | 20.40 | C |
| ATOM | 2346 | C | PHE | A | 373 | 12.472 | 21.602 | 73.189 | 1.00 | 18.33 | C |
| ATOM | 2347 | O | PHE | A | 373 | 12.602 | 22.613 | 73.844 | 1.00 | 18.29 | O |
| ATOM | 2357 | N | ILE | A | 374 | 11.743 | 21.595 | 72.097 | 1.00 | 18.65 | N |
| ATOM | 2358 | CA | ILE | A | 374 | 11.085 | 22.813 | 71.625 | 1.00 | 18.60 | C |
| ATOM | 2359 | CB | ILE | A | 374 | 10.464 | 22.589 | 70.235 | 1.00 | 17.68 | C |
| ATOM | 2360 | CG1 | ILE | A | 374 | 11.575 | 22.499 | 69.199 | 1.00 | 19.05 | C |
| ATOM | 2361 | CD1 | ILE | A | 374 | 11.147 | 21.952 | 67.845 | 1.00 | 18.93 | C |
| ATOM | 2362 | CG2 | ILE | A | 374 | 9.515 | 23.714 | 69.902 | 1.00 | 17.52 | C |
| ATOM | 2363 | C | ILE | A | 374 | 10.054 | 23.327 | 72.628 | 1.00 | 18.48 | C |
| ATOM | 2364 | O | ILE | A | 374 | 9.962 | 24.538 | 72.883 | 1.00 | 18.47 | O |
| ATOM | 2376 | N | ILE | A | 375 | 9.294 | 22.395 | 73.185 | 1.00 | 18.87 | N |
| ATOM | 2377 | CA | ILE | A | 375 | 8.283 | 22.685 | 74.194 | 1.00 | 19.24 | C |
| ATOM | 2378 | CB | ILE | A | 375 | 7.510 | 21.416 | 74.574 | 1.00 | 19.70 | C |
| ATOM | 2379 | CG1 | ILE | A | 375 | 6.640 | 20.963 | 73.410 | 1.00 | 20.22 | C |
| ATOM | 2380 | CD1 | ILE | A | 375 | 6.171 | 19.550 | 73.506 | 1.00 | 20.12 | C |
| ATOM | 2381 | CG2 | ILE | A | 375 | 6.642 | 21.649 | 75.843 | 1.00 | 19.82 | C |
| ATOM | 2382 | C | ILE | A | 375 | 8.921 | 23.248 | 75.423 | 1.00 | 19.45 | C |
| ATOM | 2383 | O | ILE | A | 375 | 8.429 | 24.206 | 75.970 | 1.00 | 20.14 | O |
| ATOM | 2395 | N | LEU | A | 376 | 10.011 | 22.632 | 75.859 | 1.00 | 20.03 | N |
| ATOM | 2396 | CA | LEU | A | 376 | 10.800 | 23.110 | 76.981 | 1.00 | 20.50 | C |
| ATOM | 2397 | CB | LEU | A | 376 | 12.063 | 22.248 | 77.155 | 1.00 | 20.96 | C |
| ATOM | 2398 | CG | LEU | A | 376 | 13.000 | 22.498 | 78.339 | 1.00 | 20.06 | C |
| ATOM | 2399 | CD1 | LEU | A | 376 | 12.269 | 22.400 | 79.639 | 1.00 | 20.14 | C |
| ATOM | 2400 | CD2 | LEU | A | 376 | 14.158 | 21.499 | 78.306 | 1.00 | 21.22 | C |
| ATOM | 2401 | C | LEU | A | 376 | 11.187 | 24.577 | 76.852 | 1.00 | 21.18 | C |
| ATOM | 2402 | O | LEU | A | 376 | 11.048 | 25.340 | 77.819 | 1.00 | 20.99 | O |
| ATOM | 2414 | N | PHE | A | 377 | 11.625 | 24.977 | 75.658 | 1.00 | 22.12 | N |
| ATOM | 2415 | CA | PHE | A | 377 | 12.079 | 26.348 | 75.402 | 1.00 | 22.30 | C |
| ATOM | 2416 | CB | PHE | A | 377 | 13.364 | 26.308 | 74.613 | 1.00 | 22.04 | C |
| ATOM | 2417 | CG | PHE | A | 377 | 14.563 | 26.016 | 75.454 | 1.00 | 22.67 | C |
| ATOM | 2418 | CD1 | PHE | A | 377 | 15.022 | 24.719 | 75.601 | 1.00 | 22.00 | C |
| ATOM | 2419 | CE1 | PHE | A | 377 | 16.152 | 24.446 | 76.400 | 1.00 | 23.73 | C |
| ATOM | 2420 | CZ | PHE | A | 377 | 16.807 | 25.473 | 77.065 | 1.00 | 22.98 | C |
| ATOM | 2421 | CE2 | PHE | A | 377 | 16.329 | 26.778 | 76.946 | 1.00 | 23.96 | C |
| ATOM | 2422 | CD2 | PHE | A | 377 | 15.215 | 27.042 | 76.137 | 1.00 | 22.40 | C |
| ATOM | 2423 | C | PHE | A | 377 | 11.057 | 27.240 | 74.701 | 1.00 | 22.99 | C |
| ATOM | 2424 | O | PHE | A | 377 | 11.397 | 28.302 | 74.212 | 1.00 | 22.76 | O |
| ATOM | 2434 | N | SER | A | 378 | 9.805 | 26.797 | 74.677 | 1.00 | 24.46 | N |
| ATOM | 2435 | CA | SER | A | 378 | 8.680 | 27.561 | 74.109 | 1.00 | 25.71 | C |
| ATOM | 2436 | CB | SER | A | 378 | 7.520 | 26.611 | 73.793 | 1.00 | 25.76 | C |
| ATOM | 2437 | OG | SER | A | 378 | 6.941 | 26.085 | 75.008 | 1.00 | 26.75 | O |
| ATOM | 2438 | C | SER | A | 378 | 8.159 | 28.623 | 75.076 | 1.00 | 25.99 | C |
| ATOM | 2439 | O | SER | A | 378 | 7.001 | 28.586 | 75.475 | 1.00 | 28.05 | O |
| ATOM | 2445 | N | LEU | A | 379 | 9.023 | 29.519 | 75.509 | 1.00 | 25.62 | N |
| ATOM | 2446 | CA | LEU | A | 379 | 8.643 | 30.602 | 76.366 | 1.00 | 25.17 | C |
| ATOM | 2447 | CB | LEU | A | 379 | 9.149 | 30.390 | 77.805 | 1.00 | 24.81 | C |
| ATOM | 2448 | CG | LEU | A | 379 | 8.933 | 31.569 | 78.791 | 1.00 | 24.46 | C |
| ATOM | 2449 | CD1 | LEU | A | 379 | 7.476 | 31.769 | 79.192 | 1.00 | 24.12 | C |
| ATOM | 2450 | CD2 | LEU | A | 379 | 9.758 | 31.419 | 80.044 | 1.00 | 24.30 | C |
| ATOM | 2451 | C | LEU | A | 379 | 9.269 | 31.826 | 75.725 | 1.00 | 25.14 | C |
| ATOM | 2452 | O | LEU | A | 379 | 10.381 | 31.757 | 75.206 | 1.00 | 24.89 | O |
| ATOM | 2464 | N | ASP | A | 380 | 8.538 | 32.935 | 75.745 | 1.00 | 25.27 | N |
| ATOM | 2465 | CA | ASP | A | 380 | 9.042 | 34.174 | 75.205 | 1.00 | 25.53 | C |
| ATOM | 2466 | CB | ASP | A | 380 | 7.964 | 35.240 | 75.171 | 1.00 | 25.99 | C |
| ATOM | 2467 | CG | ASP | A | 380 | 8.321 | 36.366 | 74.235 | 1.00 | 28.41 | C |
| ATOM | 2468 | OD1 | ASP | A | 380 | 8.046 | 37.545 | 74.552 | 1.00 | 31.53 | O |
| ATOM | 2469 | OD2 | ASP | A | 380 | 8.900 | 36.154 | 73.149 | 1.00 | 33.00 | O |
| ATOM | 2470 | C | ASP | A | 380 | 10.189 | 34.657 | 76.044 | 1.00 | 25.32 | C |
| ATOM | 2471 | O | ASP | A | 380 | 10.118 | 34.646 | 77.270 | 1.00 | 24.98 | O |
| ATOM | 2476 | N | LEU | A | 381 | 11.228 | 35.104 | 75.353 | 1.00 | 25.71 | N |
| ATOM | 2477 | CA | LEU | A | 381 | 12.499 | 35.509 | 75.944 | 1.00 | 25.96 | C |
| ATOM | 2478 | CB | LEU | A | 381 | 13.375 | 36.194 | 74.892 | 1.00 | 26.37 | C |
| ATOM | 2479 | CG | LEU | A | 381 | 13.928 | 35.316 | 73.783 | 1.00 | 27.97 | C |
| ATOM | 2480 | CD1 | LEU | A | 381 | 14.812 | 36.130 | 72.837 | 1.00 | 28.28 | C |
| ATOM | 2481 | CD2 | LEU | A | 381 | 14.692 | 34.159 | 74.411 | 1.00 | 29.64 | C |
| ATOM | 2482 | C | LEU | A | 381 | 12.375 | 36.494 | 77.061 | 1.00 | 25.39 | C |
| ATOM | 2483 | O | LEU | A | 381 | 13.193 | 36.495 | 77.968 | 1.00 | 24.91 | O |
| ATOM | 2495 | N | LYS | A | 382 | 11.390 | 37.374 | 76.943 | 1.00 | 25.24 | N |
| ATOM | 2496 | CA | LYS | A | 382 | 11.233 | 38.487 | 77.866 | 1.00 | 25.01 | C |
| ATOM | 2497 | CB | LYS | A | 382 | 10.076 | 39.409 | 77.435 | 1.00 | 25.08 | C |
| ATOM | 2498 | CG | LYS | A | 382 | 8.691 | 38.771 | 77.455 | 1.00 | 26.26 | C |
| ATOM | 2499 | CD | LYS | A | 382 | 7.690 | 39.643 | 76.695 | 1.00 | 27.61 | C |
| ATOM | 2500 | CE | LYS | A | 382 | 6.274 | 39.059 | 76.699 | 1.00 | 28.85 | C |
| ATOM | 2501 | NZ | LYS | A | 382 | 5.364 | 39.885 | 75.833 | 1.00 | 30.23 | N |
| ATOM | 2502 | C | LYS | A | 382 | 11.067 | 38.031 | 79.309 | 1.00 | 24.44 | C |
| ATOM | 2503 | O | LYS | A | 382 | 11.492 | 38.722 | 80.221 | 1.00 | 24.43 | O |
| ATOM | 2517 | N | PHE | A | 383 | 10.502 | 36.845 | 79.513 | 1.00 | 23.99 | N |
| ATOM | 2518 | CA | PHE | A | 383 | 10.249 | 36.324 | 80.862 | 1.00 | 23.57 | C |
| ATOM | 2519 | CB | PHE | A | 383 | 9.141 | 35.268 | 80.793 | 1.00 | 23.87 | C |
| ATOM | 2520 | CG | PHE | A | 383 | 7.837 | 35.791 | 80.243 | 1.00 | 25.00 | C |
| ATOM | 2521 | CD1 | PHE | A | 383 | 7.333 | 35.324 | 79.039 | 1.00 | 26.66 | C |
| ATOM | 2522 | CE1 | PHE | A | 383 | 6.114 | 35.815 | 78.526 | 1.00 | 27.19 | C |
| ATOM | 2523 | CZ | PHE | A | 383 | 5.406 | 36.778 | 79.220 | 1.00 | 27.54 | C |
| ATOM | 2524 | CE2 | PHE | A | 383 | 5.908 | 37.266 | 80.417 | 1.00 | 27.62 | C |
| ATOM | 2525 | CD2 | PHE | A | 383 | 7.122 | 36.766 | 80.925 | 1.00 | 26.88 | C |
| ATOM | 2526 | C | PHE | A | 383 | 11.489 | 35.769 | 81.599 | 1.00 | 22.90 | C |
| ATOM | 2527 | O | PHE | A | 383 | 11.402 | 35.350 | 82.742 | 1.00 | 23.03 | O |
| ATOM | 2537 | N | LEU | A | 384 | 12.646 | 35.815 | 80.964 | 1.00 | 22.63 | N |
| ATOM | 2538 | CA | LEU | A | 384 | 13.868 | 35.216 | 81.490 | 1.00 | 22.66 | C |
| ATOM | 2539 | CB | LEU | A | 384 | 14.286 | 34.058 | 80.581 | 1.00 | 22.60 | C |
| ATOM | 2540 | CG | LEU | A | 384 | 13.219 | 32.984 | 80.437 | 1.00 | 23.54 | C |
| ATOM | 2541 | CD1 | LEU | A | 384 | 13.544 | 32.029 | 79.311 | 1.00 | 25.61 | C |
| ATOM | 2542 | CD2 | LEU | A | 384 | 13.057 | 32.232 | 81.736 | 1.00 | 23.44 | C |
| ATOM | 2543 | C | LEU | A | 384 | 15.009 | 36.221 | 81.551 | 1.00 | 22.32 | C |
| ATOM | 2544 | O | LEU | A | 384 | 14.980 | 37.250 | 80.863 | 1.00 | 22.67 | O |
| ATOM | 2556 | N | ASN | A | 385 | 16.013 | 35.907 | 82.369 | 1.00 | 22.09 | N |
| ATOM | 2557 | CA | ASN | A | 385 | 17.223 | 36.714 | 82.480 | 1.00 | 21.97 | C |
| ATOM | 2558 | CB | ASN | A | 385 | 18.057 | 36.291 | 83.678 | 1.00 | 21.83 | C |
| ATOM | 2559 | CG | ASN | A | 385 | 17.345 | 36.465 | 84.974 | 1.00 | 21.43 | C |
| ATOM | 2560 | OD1 | ASN | A | 385 | 16.868 | 37.552 | 85.307 | 1.00 | 21.64 | O |
| ATOM | 2561 | ND2 | ASN | A | 385 | 17.307 | 35.398 | 85.749 | 1.00 | 20.32 | N |
| ATOM | 2562 | C | ASN | A | 385 | 18.099 | 36.535 | 81.273 | 1.00 | 21.97 | C |
| ATOM | 2563 | O | ASN | A | 385 | 18.561 | 37.492 | 80.661 | 1.00 | 21.84 | O |
| ATOM | 2570 | N | ASN | A | 386 | 18.355 | 35.281 | 80.957 | 1.00 | 22.62 | N |
| ATOM | 2571 | CA | ASN | A | 386 | 19.271 | 34.946 | 79.888 | 1.00 | 23.32 | C |
| ATOM | 2572 | CB | ASN | A | 386 | 19.996 | 33.636 | 80.219 | 1.00 | 23.43 | C |
| ATOM | 2573 | CG | ASN | A | 386 | 21.243 | 33.430 | 79.384 | 1.00 | 23.81 | C |
| ATOM | 2574 | OD1 | ASN | A | 386 | 21.267 | 33.764 | 78.211 | 1.00 | 26.86 | O |
| ATOM | 2575 | ND2 | ASN | A | 386 | 22.280 | 32.885 | 79.987 | 1.00 | 22.52 | N |
| ATOM | 2576 | C | ASN | A | 386 | 18.548 | 34.851 | 78.551 | 1.00 | 23.68 | C |
| ATOM | 2577 | O | ASN | A | 386 | 18.037 | 33.806 | 78.202 | 1.00 | 24.08 | O |
| ATOM | 2584 | N | HIS | A | 387 | 18.520 | 35.951 | 77.806 | 1.00 | 24.33 | N |
| ATOM | 2585 | CA | HIS | A | 387 | 17.990 | 35.944 | 76.440 | 1.00 | 24.77 | C |
| ATOM | 2586 | CB | HIS | A | 387 | 17.830 | 37.363 | 75.858 | 1.00 | 24.89 | C |
| ATOM | 2587 | CG | HIS | A | 387 | 17.144 | 38.314 | 76.773 | 1.00 | 24.42 | C |
| ATOM | 2588 | ND1 | HIS | A | 387 | 17.457 | 39.574 | 77.145 | 1.00 | 25.80 | N |
| ATOM | 2589 | CE1 | HIS | A | 387 | 16.495 | 39.982 | 78.032 | 1.00 | 26.12 | C |
| ATOM | 2590 | NE2 | HIS | A | 387 | 15.626 | 39.002 | 78.203 | 1.00 | 25.87 | N |
| ATOM | 2591 | CD2 | HIS | A | 387 | 16.007 | 37.977 | 77.466 | 1.00 | 24.97 | C |
| ATOM | 2592 | C | HIS | A | 387 | 18.911 | 35.193 | 75.503 | 1.00 | 25.04 | C |
| ATOM | 2593 | O | HIS | A | 387 | 18.458 | 34.328 | 74.780 | 1.00 | 25.70 | O |
| ATOM | 2602 | N | ILE | A | 388 | 20.191 | 35.553 | 75.504 | 1.00 | 25.09 | N |
| ATOM | 2603 | CA | ILE | A | 388 | 21.114 | 35.091 | 74.473 | 1.00 | 25.53 | C |
| ATOM | 2604 | CB | ILE | A | 388 | 22.581 | 35.577 | 74.749 | 1.00 | 25.64 | C |
| ATOM | 2605 | CG1 | ILE | A | 388 | 23.563 | 34.990 | 73.736 | 1.00 | 25.89 | C |
| ATOM | 2606 | CD1 | ILE | A | 388 | 24.894 | 35.787 | 73.619 | 1.00 | 26.15 | C |
| ATOM | 2607 | CG2 | ILE | A | 388 | 23.064 | 35.214 | 76.172 | 1.00 | 26.98 | C |
| ATOM | 2608 | C | ILE | A | 388 | 21.058 | 33.578 | 74.256 | 1.00 | 25.27 | C |
| ATOM | 2609 | O | ILE | A | 388 | 20.988 | 33.112 | 73.126 | 1.00 | 25.36 | O |
| ATOM | 2621 | N | LEU | A | 389 | 21.072 | 32.814 | 75.330 | 1.00 | 25.17 | N |
| ATOM | 2622 | CA | LEU | A | 389 | 21.192 | 31.365 | 75.206 | 1.00 | 25.46 | C |
| ATOM | 2623 | CB | LEU | A | 389 | 21.740 | 30.749 | 76.490 | 1.00 | 25.78 | C |
| ATOM | 2624 | CG | LEU | A | 389 | 23.206 | 31.085 | 76.744 | 1.00 | 26.48 | C |
| ATOM | 2625 | CD1 | LEU | A | 389 | 23.589 | 30.466 | 78.080 | 1.00 | 27.92 | C |
| ATOM | 2626 | CD2 | LEU | A | 389 | 24.111 | 30.583 | 75.627 | 1.00 | 27.22 | C |
| ATOM | 2627 | C | LEU | A | 389 | 19.883 | 30.700 | 74.866 | 1.00 | 25.08 | C |
| ATOM | 2628 | O | LEU | A | 389 | 19.868 | 29.733 | 74.103 | 1.00 | 24.94 | O |
| ATOM | 2640 | N | VAL | A | 390 | 18.798 | 31.217 | 75.432 | 1.00 | 24.57 | N |
| ATOM | 2641 | CA | VAL | A | 390 | 17.477 | 30.683 | 75.150 | 1.00 | 24.63 | C |
| ATOM | 2642 | CB | VAL | A | 390 | 16.415 | 31.265 | 76.105 | 1.00 | 24.41 | C |
| ATOM | 2643 | CG1 | VAL | A | 390 | 15.015 | 30.843 | 75.693 | 1.00 | 24.69 | C |
| ATOM | 2644 | CG2 | VAL | A | 390 | 16.681 | 30.827 | 77.529 | 1.00 | 24.67 | C |
| ATOM | 2645 | C | VAL | A | 390 | 17.055 | 30.928 | 73.690 | 1.00 | 24.69 | C |
| ATOM | 2646 | O | VAL | A | 390 | 16.381 | 30.085 | 73.091 | 1.00 | 25.10 | O |
| ATOM | 2656 | N | LYS | A | 391 | 17.424 | 32.079 | 73.133 | 1.00 | 24.43 | N |
| ATOM | 2657 | CA | LYS | A | 391 | 17.092 | 32.418 | 71.756 | 1.00 | 24.07 | C |
| ATOM | 2658 | CB | LYS | A | 391 | 17.530 | 33.852 | 71.455 | 1.00 | 24.46 | C |
| ATOM | 2659 | CG | LYS | A | 391 | 17.271 | 34.337 | 70.018 | 1.00 | 24.30 | C |
| ATOM | 2660 | CD | LYS | A | 391 | 17.460 | 35.837 | 69.938 | 1.00 | 24.65 | C |
| ATOM | 2661 | CE | LYS | A | 391 | 17.039 | 36.405 | 68.592 | 1.00 | 25.13 | C |
| ATOM | 2662 | NZ | LYS | A | 391 | 18.147 | 37.127 | 67.942 | 1.00 | 24.33 | N |
| ATOM | 2663 | C | LYS | A | 391 | 17.816 | 31.484 | 70.815 | 1.00 | 24.11 | C |
| ATOM | 2664 | O | LYS | A | 391 | 17.262 | 31.039 | 69.821 | 1.00 | 23.59 | O |
| ATOM | 2678 | N | ASP | A | 392 | 19.072 | 31.216 | 71.136 | 1.00 | 24.20 | N |
| ATOM | 2679 | CA | ASP | A | 392 | 19.885 | 30.330 | 70.349 | 1.00 | 24.86 | C |
| ATOM | 2680 | CB | ASP | A | 392 | 21.311 | 30.318 | 70.877 | 1.00 | 25.07 | C |
| ATOM | 2681 | CG | ASP | A | 392 | 22.235 | 29.503 | 70.008 | 1.00 | 27.58 | C |
| ATOM | 2682 | OD1 | ASP | A | 392 | 22.239 | 29.713 | 68.769 | 1.00 | 29.43 | O |
| ATOM | 2683 | OD2 | ASP | A | 392 | 22.997 | 28.622 | 70.469 | 1.00 | 32.58 | O |
| ATOM | 2684 | C | ASP | A | 392 | 19.302 | 28.919 | 70.359 | 1.00 | 24.69 | C |
| ATOM | 2685 | O | ASP | A | 392 | 19.244 | 28.256 | 69.327 | 1.00 | 25.06 | O |
| ATOM | 2690 | N | ALA | A | 393 | 18.873 | 28.467 | 71.528 | 1.00 | 24.15 | N |
| ATOM | 2691 | CA | ALA | A | 393 | 18.264 | 27.152 | 71.667 | 1.00 | 23.68 | C |
| ATOM | 2692 | CB | ALA | A | 393 | 18.013 | 26.852 | 73.114 | 1.00 | 23.83 | C |
| ATOM | 2693 | C | ALA | A | 393 | 16.966 | 27.070 | 70.900 | 1.00 | 23.18 | C |
| ATOM | 2694 | O | ALA | A | 393 | 16.696 | 26.085 | 70.262 | 1.00 | 23.38 | O |
| ATOM | 2700 | N | GLN | A | 394 | 16.166 | 28.119 | 70.958 | 1.00 | 22.93 | N |
| ATOM | 2701 | CA | GLN | A | 394 | 14.878 | 28.138 | 70.272 | 1.00 | 22.68 | C |
| ATOM | 2702 | CB | GLN | A | 394 | 14.124 | 29.422 | 70.594 | 1.00 | 22.83 | C |
| ATOM | 2703 | CG | GLN | A | 394 | 13.471 | 29.451 | 71.969 | 1.00 | 24.07 | C |
| ATOM | 2704 | CD | GLN | A | 394 | 13.039 | 30.845 | 72.399 | 1.00 | 24.29 | C |
| ATOM | 2705 | OE1 | GLN | A | 394 | 13.276 | 31.833 | 71.689 | 1.00 | 26.27 | O |
| ATOM | 2706 | NE2 | GLN | A | 394 | 12.398 | 30.927 | 73.556 | 1.00 | 24.58 | N |
| ATOM | 2707 | C | GLN | A | 394 | 15.060 | 28.057 | 68.775 | 1.00 | 22.11 | C |
| ATOM | 2708 | O | GLN | A | 394 | 14.291 | 27.396 | 68.095 | 1.00 | 21.72 | O |
| ATOM | 2717 | N | GLU | A | 395 | 16.067 | 28.771 | 68.285 | 1.00 | 21.76 | N |
| ATOM | 2718 | CA | GLU | A | 395 | 16.373 | 28.856 | 66.860 | 1.00 | 21.91 | C |
| ATOM | 2719 | CB | GLU | A | 395 | 17.295 | 30.058 | 66.573 | 1.00 | 21.92 | C |
| ATOM | 2720 | CG | GLU | A | 395 | 16.626 | 31.410 | 66.790 | 1.00 | 22.49 | C |
| ATOM | 2721 | CD | GLU | A | 395 | 17.539 | 32.586 | 66.475 | 1.00 | 23.61 | C |
| ATOM | 2722 | OE1 | GLU | A | 395 | 18.774 | 32.421 | 66.502 | 1.00 | 24.02 | O |
| ATOM | 2723 | OE2 | GLU | A | 395 | 17.019 | 33.689 | 66.207 | 1.00 | 25.17 | O |
| ATOM | 2724 | C | GLU | A | 395 | 17.017 | 27.559 | 66.330 | 1.00 | 21.70 | C |
| ATOM | 2725 | O | GLU | A | 395 | 16.610 | 27.039 | 65.294 | 1.00 | 19.95 | O |
| ATOM | 2732 | N | LYS | A | 396 | 18.018 | 27.069 | 67.062 | 1.00 | 21.77 | N |
| ATOM | 2733 | CA | LYS | A | 396 | 18.705 | 25.838 | 66.729 | 1.00 | 22.42 | C |
| ATOM | 2734 | CB | LYS | A | 396 | 19.849 | 25.598 | 67.710 | 1.00 | 22.60 | C |
| ATOM | 2735 | CG | LYS | A | 396 | 20.969 | 26.637 | 67.627 | 1.00 | 24.93 | C |
| ATOM | 2736 | CD | LYS | A | 396 | 22.153 | 26.195 | 66.756 | 1.00 | 26.69 | C |
| ATOM | 2737 | CE | LYS | A | 396 | 23.325 | 27.195 | 66.832 | 1.00 | 27.39 | C |
| ATOM | 2738 | NZ | LYS | A | 396 | 24.249 | 26.967 | 67.995 | 1.00 | 27.30 | N |
| ATOM | 2739 | C | LYS | A | 396 | 17.758 | 24.629 | 66.745 | 1.00 | 22.38 | C |
| ATOM | 2740 | O | LYS | A | 396 | 17.947 | 23.681 | 65.974 | 1.00 | 22.88 | O |
| ATOM | 2754 | N | ALA | A | 397 | 16.748 | 24.664 | 67.613 | 1.00 | 21.70 | N |
| ATOM | 2755 | CA | ALA | A | 397 | 15.843 | 23.534 | 67.766 | 1.00 | 21.29 | C |
| ATOM | 2756 | CB | ALA | A | 397 | 15.116 | 23.609 | 69.092 | 1.00 | 21.44 | C |
| ATOM | 2757 | C | ALA | A | 397 | 14.856 | 23.437 | 66.606 | 1.00 | 20.75 | C |
| ATOM | 2758 | O | ALA | A | 397 | 14.562 | 22.352 | 66.125 | 1.00 | 19.34 | O |
| ATOM | 2764 | N | ASN | A | 398 | 14.351 | 24.580 | 66.167 | 1.00 | 20.73 | N |
| ATOM | 2765 | CA | ASN | A | 398 | 13.471 | 24.638 | 65.014 | 1.00 | 21.27 | C |
| ATOM | 2766 | CB | ASN | A | 398 | 12.882 | 26.044 | 64.875 | 1.00 | 22.09 | C |
| ATOM | 2767 | CG | ASN | A | 398 | 11.553 | 26.197 | 65.588 | 1.00 | 24.23 | C |
| ATOM | 2768 | OD1 | ASN | A | 398 | 11.019 | 25.254 | 66.196 | 1.00 | 26.64 | O |
| ATOM | 2769 | ND2 | ASN | A | 398 | 11.012 | 27.402 | 65.530 | 1.00 | 27.30 | N |
| ATOM | 2770 | C | ASN | A | 398 | 14.184 | 24.306 | 63.712 | 1.00 | 20.32 | C |
| ATOM | 2771 | O | ASN | A | 398 | 13.632 | 23.614 | 62.847 | 1.00 | 19.91 | O |
| ATOM | 2778 | N | ALA | A | 399 | 15.399 | 24.831 | 63.573 | 1.00 | 19.59 | N |
| ATOM | 2779 | CA | ALA | A | 399 | 16.232 | 24.568 | 62.411 | 1.00 | 19.30 | C |
| ATOM | 2780 | CB | ALA | A | 399 | 17.545 | 25.348 | 62.498 | 1.00 | 19.12 | C |
| ATOM | 2781 | C | ALA | A | 399 | 16.522 | 23.084 | 62.288 | 1.00 | 18.71 | C |
| ATOM | 2782 | O | ALA | A | 399 | 16.544 | 22.541 | 61.190 | 1.00 | 18.75 | O |
| ATOM | 2788 | N | ALA | A | 400 | 16.759 | 22.455 | 63.429 | 1.00 | 18.21 | N |
| ATOM | 2789 | CA | ALA | A | 400 | 17.028 | 21.030 | 63.494 | 1.00 | 18.38 | C |
| ATOM | 2790 | CB | ALA | A | 400 | 17.444 | 20.610 | 64.910 | 1.00 | 18.33 | C |
| ATOM | 2791 | C | ALA | A | 400 | 15.838 | 20.228 | 63.044 | 1.00 | 17.84 | C |
| ATOM | 2792 | O | ALA | A | 400 | 16.004 | 19.277 | 62.279 | 1.00 | 18.31 | O |
| ATOM | 2798 | N | LEU | A | 401 | 14.649 | 20.627 | 63.481 | 1.00 | 17.21 | N |
| ATOM | 2799 | CA | LEU | A | 401 | 13.437 | 19.901 | 63.127 | 1.00 | 17.33 | C |
| ATOM | 2800 | CB | LEU | A | 401 | 12.235 | 20.409 | 63.933 | 1.00 | 17.09 | C |
| ATOM | 2801 | CG | LEU | A | 401 | 10.889 | 19.694 | 63.729 | 1.00 | 17.94 | C |
| ATOM | 2802 | CD1 | LEU | A | 401 | 10.898 | 18.250 | 64.242 | 1.00 | 19.26 | C |
| ATOM | 2803 | CD2 | LEU | A | 401 | 9.715 | 20.449 | 64.330 | 1.00 | 17.99 | C |
| ATOM | 2804 | C | LEU | A | 401 | 13.175 | 19.995 | 61.621 | 1.00 | 17.31 | C |
| ATOM | 2805 | O | LEU | A | 401 | 12.845 | 19.011 | 60.983 | 1.00 | 17.20 | O |
| ATOM | 2817 | N | LEU | A | 402 | 13.353 | 21.186 | 61.060 | 1.00 | 17.89 | N |
| ATOM | 2818 | CA | LEU | A | 402 | 13.138 | 21.429 | 59.647 | 1.00 | 17.74 | C |
| ATOM | 2819 | CB | LEU | A | 402 | 13.366 | 22.909 | 59.338 | 1.00 | 17.93 | C |
| ATOM | 2820 | CG | LEU | A | 402 | 12.796 | 23.495 | 58.025 | 1.00 | 18.15 | C |
| ATOM | 2821 | CD1 | LEU | A | 402 | 13.859 | 23.991 | 57.118 | 1.00 | 18.39 | C |
| ATOM | 2822 | CD2 | LEU | A | 402 | 11.850 | 22.560 | 57.267 | 1.00 | 17.97 | C |
| ATOM | 2823 | C | LEU | A | 402 | 14.096 | 20.627 | 58.802 | 1.00 | 17.79 | C |
| ATOM | 2824 | O | LEU | A | 402 | 13.729 | 20.012 | 57.817 | 1.00 | 17.11 | O |
| ATOM | 2836 | N | ASP | A | 403 | 15.347 | 20.662 | 59.206 | 1.00 | 18.07 | N |
| ATOM | 2837 | CA | ASP | A | 403 | 16.389 | 19.985 | 58.491 | 1.00 | 18.39 | C |
| ATOM | 2838 | CB | ASP | A | 403 | 17.726 | 20.243 | 59.166 | 1.00 | 18.69 | C |
| ATOM | 2839 | CG | ASP | A | 403 | 18.865 | 19.843 | 58.322 | 1.00 | 19.23 | C |
| ATOM | 2840 | OD1 | ASP | A | 403 | 19.400 | 18.731 | 58.534 | 1.00 | 21.05 | O |
| ATOM | 2841 | OD2 | ASP | A | 403 | 19.278 | 20.582 | 57.407 | 1.00 | 21.58 | O |
| ATOM | 2842 | C | ASP | A | 403 | 16.093 | 18.505 | 58.457 | 1.00 | 18.36 | C |
| ATOM | 2843 | O | ASP | A | 403 | 16.226 | 17.893 | 57.410 | 1.00 | 18.43 | O |
| ATOM | 2848 | N | TYR | A | 404 | 15.660 | 17.944 | 59.587 | 1.00 | 18.19 | N |
| ATOM | 2849 | CA | TYR | A | 404 | 15.412 | 16.503 | 59.686 | 1.00 | 18.39 | C |
| ATOM | 2850 | CB | TYR | A | 404 | 15.291 | 16.067 | 61.153 | 1.00 | 18.42 | C |
| ATOM | 2851 | CG | TYR | A | 404 | 14.993 | 14.599 | 61.323 | 1.00 | 17.30 | C |
| ATOM | 2852 | CD1 | TYR | A | 404 | 16.018 | 13.675 | 61.430 | 1.00 | 16.30 | C |
| ATOM | 2853 | CE1 | TYR | A | 404 | 15.754 | 12.333 | 61.563 | 1.00 | 16.45 | C |
| ATOM | 2854 | CZ | TYR | A | 404 | 14.449 | 11.907 | 61.595 | 1.00 | 17.69 | C |
| ATOM | 2855 | OH | TYR | A | 404 | 14.178 | 10.577 | 61.721 | 1.00 | 19.68 | O |
| ATOM | 2856 | CE2 | TYR | A | 404 | 13.408 | 12.810 | 61.494 | 1.00 | 18.10 | C |
| ATOM | 2857 | CD2 | TYR | A | 404 | 13.688 | 14.142 | 61.359 | 1.00 | 17.53 | C |
| ATOM | 2858 | C | TYR | A | 404 | 14.180 | 16.063 | 58.879 | 1.00 | 18.55 | C |
| ATOM | 2859 | O | TYR | A | 404 | 14.241 | 15.109 | 58.128 | 1.00 | 18.35 | O |
| ATOM | 2869 | N | THR | A | 405 | 13.081 | 16.789 | 59.013 | 1.00 | 19.24 | N |
| ATOM | 2870 | CA | THR | A | 405 | 11.823 | 16.464 | 58.327 | 1.00 | 19.72 | C |
| ATOM | 2871 | CB | THR | A | 405 | 10.674 | 17.383 | 58.811 | 1.00 | 19.76 | C |
| ATOM | 2872 | OG1 | THR | A | 405 | 11.060 | 18.763 | 58.729 | 1.00 | 20.43 | O |
| ATOM | 2873 | CG2 | THR | A | 405 | 10.380 | 17.167 | 60.266 | 1.00 | 20.30 | C |
| ATOM | 2874 | C | THR | A | 405 | 11.952 | 16.623 | 56.843 | 1.00 | 19.76 | C |
| ATOM | 2875 | O | THR | A | 405 | 11.335 | 15.907 | 56.079 | 1.00 | 20.31 | O |
| ATOM | 2883 | N | LEU | A | 406 | 12.754 | 17.589 | 56.435 | 1.00 | 20.28 | N |
| ATOM | 2884 | CA | LEU | A | 406 | 12.972 | 17.871 | 55.028 | 1.00 | 20.72 | C |
| ATOM | 2885 | CB | LEU | A | 406 | 13.685 | 19.219 | 54.898 | 1.00 | 20.95 | C |
| ATOM | 2886 | CG | LEU | A | 406 | 13.165 | 20.295 | 53.943 | 1.00 | 21.77 | C |
| ATOM | 2887 | CD1 | LEU | A | 406 | 11.660 | 20.374 | 53.914 | 1.00 | 22.46 | C |
| ATOM | 2888 | CD2 | LEU | A | 406 | 13.785 | 21.648 | 54.309 | 1.00 | 21.82 | C |
| ATOM | 2889 | C | LEU | A | 406 | 13.773 | 16.737 | 54.339 | 1.00 | 20.73 | C |
| ATOM | 2890 | O | LEU | A | 406 | 13.492 | 16.379 | 53.205 | 1.00 | 19.98 | O |
| ATOM | 2902 | N | CYS | A | 407 | 14.722 | 16.162 | 55.073 | 1.00 | 21.27 | N |
| ATOM | 2903 | CA | CYS | A | 407 | 15.601 | 15.104 | 54.604 | 1.00 | 21.98 | C |
| ATOM | 2904 | CB | CYS | A | 407 | 16.898 | 15.147 | 55.405 | 1.00 | 21.71 | C |
| ATOM | 2905 | SG | CYS | A | 407 | 17.979 | 16.555 | 55.037 | 1.00 | 22.56 | S |
| ATOM | 2906 | C | CYS | A | 407 | 15.022 | 13.694 | 54.729 | 1.00 | 22.93 | C |
| ATOM | 2907 | O | CYS | A | 407 | 15.429 | 12.783 | 54.009 | 1.00 | 23.48 | O |
| ATOM | 2913 | N | HIS | A | 408 | 14.091 | 13.507 | 55.649 | 1.00 | 24.40 | N |
| ATOM | 2914 | CA | HIS | A | 408 | 13.660 | 12.167 | 56.059 | 1.00 | 25.54 | C |
| ATOM | 2915 | CB | HIS | A | 408 | 13.881 | 11.969 | 57.575 | 1.00 | 25.12 | C |
| ATOM | 2916 | CG | HIS | A | 408 | 15.330 | 11.945 | 57.942 | 1.00 | 24.87 | C |
| ATOM | 2917 | ND1 | HIS | A | 408 | 16.181 | 12.938 | 58.302 | 1.00 | 25.13 | N |
| ATOM | 2918 | CE1 | HIS | A | 408 | 17.424 | 12.374 | 58.448 | 1.00 | 25.95 | C |
| ATOM | 2919 | NE2 | HIS | A | 408 | 17.347 | 11.088 | 58.151 | 1.00 | 25.53 | N |
| ATOM | 2920 | CD2 | HIS | A | 408 | 16.098 | 10.809 | 57.822 | 1.00 | 24.61 | C |
| ATOM | 2921 | C | HIS | A | 408 | 12.231 | 11.878 | 55.651 | 1.00 | 26.80 | C |
| ATOM | 2922 | O | HIS | A | 408 | 11.852 | 10.717 | 55.491 | 1.00 | 27.52 | O |
| ATOM | 2931 | N | TYR | A | 409 | 11.464 | 12.939 | 55.440 | 1.00 | 28.12 | N |
| ATOM | 2932 | CA | TYR | A | 409 | 10.076 | 12.853 | 55.017 | 1.00 | 29.13 | C |
| ATOM | 2933 | CB | TYR | A | 409 | 9.177 | 13.456 | 56.096 | 1.00 | 29.19 | C |
| ATOM | 2934 | CG | TYR | A | 409 | 7.680 | 13.168 | 55.993 | 1.00 | 30.74 | C |
| ATOM | 2935 | CD1 | TYR | A | 409 | 7.118 | 12.099 | 56.689 | 1.00 | 31.34 | C |
| ATOM | 2936 | CE1 | TYR | A | 409 | 5.742 | 11.848 | 56.642 | 1.00 | 32.04 | C |
| ATOM | 2937 | CZ | TYR | A | 409 | 4.895 | 12.676 | 55.902 | 1.00 | 32.68 | C |
| ATOM | 2938 | OH | TYR | A | 409 | 3.532 | 12.380 | 55.889 | 1.00 | 34.50 | O |
| ATOM | 2939 | CE2 | TYR | A | 409 | 5.419 | 13.764 | 55.203 | 1.00 | 31.24 | C |
| ATOM | 2940 | CD2 | TYR | A | 409 | 6.810 | 14.010 | 55.255 | 1.00 | 31.82 | C |
| ATOM | 2941 | C | TYR | A | 409 | 9.988 | 13.644 | 53.717 | 1.00 | 29.66 | C |
| ATOM | 2942 | O | TYR | A | 409 | 9.519 | 14.789 | 53.709 | 1.00 | 30.19 | O |
| ATOM | 2952 | N | PRO | A | 410 | 10.446 | 13.046 | 52.618 | 1.00 | 30.13 | N |
| ATOM | 2953 | CA | PRO | A | 410 | 10.390 | 13.722 | 51.309 | 1.00 | 30.52 | C |
| ATOM | 2954 | CB | PRO | A | 410 | 11.524 | 13.050 | 50.515 | 1.00 | 30.48 | C |
| ATOM | 2955 | CG | PRO | A | 410 | 11.640 | 11.643 | 51.130 | 1.00 | 30.57 | C |
| ATOM | 2956 | CD | PRO | A | 410 | 11.014 | 11.687 | 52.511 | 1.00 | 30.11 | C |
| ATOM | 2957 | C | PRO | A | 410 | 9.005 | 13.539 | 50.657 | 1.00 | 30.55 | C |
| ATOM | 2958 | O | PRO | A | 410 | 8.822 | 13.620 | 49.433 | 1.00 | 31.52 | O |
| ATOM | 2966 | N | HIS | A | 411 | 8.019 | 13.341 | 51.523 | 1.00 | 30.50 | N |
| ATOM | 2967 | CA | HIS | A | 411 | 6.654 | 13.089 | 51.133 | 1.00 | 30.19 | C |
| ATOM | 2968 | CB | HIS | A | 411 | 6.090 | 12.060 | 52.109 | 1.00 | 30.33 | C |
| ATOM | 2969 | CG | HIS | A | 411 | 6.903 | 10.803 | 52.148 | 1.00 | 32.02 | C |
| ATOM | 2970 | ND1 | HIS | A | 411 | 6.796 | 9.866 | 53.156 | 1.00 | 33.58 | N |
| ATOM | 2971 | CE1 | HIS | A | 411 | 7.626 | 8.866 | 52.905 | 1.00 | 33.80 | C |
| ATOM | 2972 | NE2 | HIS | A | 411 | 8.277 | 9.128 | 51.783 | 1.00 | 32.45 | N |
| ATOM | 2973 | CD2 | HIS | A | 411 | 7.844 | 10.332 | 51.288 | 1.00 | 31.89 | C |
| ATOM | 2974 | C | HIS | A | 411 | 5.845 | 14.387 | 51.092 | 1.00 | 29.33 | C |
| ATOM | 2975 | O | HIS | A | 411 | 6.358 | 15.476 | 51.385 | 1.00 | 29.45 | O |
| ATOM | 2984 | N | SER | A | 412 | 4.585 | 14.272 | 50.691 | 1.00 | 28.16 | N |
| ATOM | 2985 | CA | SER | A | 412 | 3.746 | 15.446 | 50.516 | 1.00 | 26.61 | C |
| ATOM | 2986 | CB | SER | A | 412 | 2.498 | 15.126 | 49.667 | 1.00 | 26.56 | C |
| ATOM | 2987 | OG | SER | A | 412 | 1.342 | 15.028 | 50.475 | 1.00 | 26.34 | O |
| ATOM | 2988 | C | SER | A | 412 | 3.390 | 15.976 | 51.895 | 1.00 | 24.90 | C |
| ATOM | 2989 | O | SER | A | 412 | 3.464 | 15.263 | 52.906 | 1.00 | 25.13 | O |
| ATOM | 2995 | N | GLY | A | 413 | 3.038 | 17.251 | 51.921 | 1.00 | 22.83 | N |
| ATOM | 2996 | CA | GLY | A | 413 | 2.794 | 17.964 | 53.149 | 1.00 | 20.85 | C |
| ATOM | 2997 | C | GLY | A | 413 | 4.098 | 18.488 | 53.688 | 1.00 | 19.27 | C |
| ATOM | 2998 | O | GLY | A | 413 | 5.114 | 17.801 | 53.684 | 1.00 | 18.32 | O |
| ATOM | 3002 | N | ASP | A | 414 | 4.046 | 19.705 | 54.203 | 1.00 | 18.12 | N |
| ATOM | 3003 | CA | ASP | A | 414 | 5.158 | 20.293 | 54.947 | 1.00 | 17.08 | C |
| ATOM | 3004 | CB | ASP | A | 414 | 5.057 | 21.810 | 54.833 | 1.00 | 16.59 | C |
| ATOM | 3005 | CG | ASP | A | 414 | 6.122 | 22.525 | 55.574 | 1.00 | 15.14 | C |
| ATOM | 3006 | OD1 | ASP | A | 414 | 6.745 | 21.940 | 56.472 | 1.00 | 13.97 | O |
| ATOM | 3007 | OD2 | ASP | A | 414 | 6.402 | 23.707 | 55.332 | 1.00 | 15.29 | O |
| ATOM | 3008 | C | ASP | A | 414 | 5.121 | 19.773 | 56.403 | 1.00 | 17.21 | C |
| ATOM | 3009 | O | ASP | A | 414 | 4.345 | 20.238 | 57.251 | 1.00 | 16.62 | O |
| ATOM | 3014 | N | LYS | A | 415 | 5.974 | 18.792 | 56.677 | 1.00 | 17.29 | N |
| ATOM | 3015 | CA | LYS | A | 415 | 5.931 | 18.060 | 57.936 | 1.00 | 17.45 | C |
| ATOM | 3016 | CB | LYS | A | 415 | 6.730 | 16.748 | 57.863 | 1.00 | 17.29 | C |
| ATOM | 3017 | CG | LYS | A | 415 | 6.694 | 15.963 | 59.167 | 1.00 | 17.76 | C |
| ATOM | 3018 | CD | LYS | A | 415 | 6.885 | 14.488 | 58.983 | 1.00 | 18.61 | C |
| ATOM | 3019 | CE | LYS | A | 415 | 7.034 | 13.772 | 60.331 | 1.00 | 19.03 | C |
| ATOM | 3020 | NZ | LYS | A | 415 | 7.674 | 12.427 | 60.235 | 1.00 | 17.38 | N |
| ATOM | 3021 | C | LYS | A | 415 | 6.387 | 18.893 | 59.123 | 1.00 | 17.45 | C |
| ATOM | 3022 | O | LYS | A | 415 | 5.871 | 18.737 | 60.207 | 1.00 | 17.58 | O |
| ATOM | 3036 | N | PHE | A | 416 | 7.371 | 19.747 | 58.913 | 1.00 | 17.58 | N |
| ATOM | 3037 | CA | PHE | A | 416 | 7.787 | 20.721 | 59.897 | 1.00 | 18.01 | C |
| ATOM | 3038 | CB | PHE | A | 416 | 8.830 | 21.632 | 59.259 | 1.00 | 17.90 | C |
| ATOM | 3039 | CG | PHE | A | 416 | 9.221 | 22.817 | 60.098 | 1.00 | 17.94 | C |
| ATOM | 3040 | CD1 | PHE | A | 416 | 10.047 | 22.656 | 61.223 | 1.00 | 17.60 | C |
| ATOM | 3041 | CE1 | PHE | A | 416 | 10.433 | 23.744 | 61.991 | 1.00 | 17.05 | C |
| ATOM | 3042 | CZ | PHE | A | 416 | 10.007 | 25.030 | 61.631 | 1.00 | 17.24 | C |
| ATOM | 3043 | CE2 | PHE | A | 416 | 9.179 | 25.214 | 60.513 | 1.00 | 16.33 | C |
| ATOM | 3044 | CD2 | PHE | A | 416 | 8.796 | 24.104 | 59.745 | 1.00 | 17.04 | C |
| ATOM | 3045 | C | PHE | A | 416 | 6.604 | 21.547 | 60.404 | 1.00 | 18.34 | C |
| ATOM | 3046 | O | PHE | A | 416 | 6.376 | 21.641 | 61.589 | 1.00 | 18.38 | O |
| ATOM | 3056 | N | GLN | A | 417 | 5.872 | 22.137 | 59.478 | 1.00 | 19.03 | N |
| ATOM | 3057 | CA | GLN | A | 417 | 4.755 | 22.986 | 59.793 | 1.00 | 19.91 | C |
| ATOM | 3058 | CB | GLN | A | 417 | 4.287 | 23.643 | 58.498 | 1.00 | 20.13 | C |
| ATOM | 3059 | CG | GLN | A | 417 | 3.310 | 24.770 | 58.664 | 1.00 | 22.66 | C |
| ATOM | 3060 | CD | GLN | A | 417 | 3.649 | 25.669 | 59.847 | 1.00 | 26.76 | C |
| ATOM | 3061 | OE1 | GLN | A | 417 | 4.724 | 26.290 | 59.865 | 1.00 | 28.30 | O |
| ATOM | 3062 | NE2 | GLN | A | 417 | 2.745 | 25.715 | 60.856 | 1.00 | 30.74 | N |
| ATOM | 3063 | C | GLN | A | 417 | 3.594 | 22.220 | 60.460 | 1.00 | 20.39 | C |
| ATOM | 3064 | O | GLN | A | 417 | 2.947 | 22.720 | 61.396 | 1.00 | 20.50 | O |
| ATOM | 3073 | N | GLN | A | 418 | 3.344 | 21.002 | 59.984 | 1.00 | 20.56 | N |
| ATOM | 3074 | CA | GLN | A | 418 | 2.287 | 20.143 | 60.507 | 1.00 | 20.58 | C |
| ATOM | 3075 | CB | GLN | A | 418 | 2.161 | 18.881 | 59.654 | 1.00 | 20.94 | C |
| ATOM | 3076 | CG | GLN | A | 418 | 1.547 | 19.118 | 58.268 | 1.00 | 24.25 | C |
| ATOM | 3077 | CD | GLN | A | 418 | 1.649 | 17.894 | 57.319 | 1.00 | 29.18 | C |
| ATOM | 3078 | OE1 | GLN | A | 418 | 2.665 | 17.190 | 57.296 | 1.00 | 33.32 | O |
| ATOM | 3079 | NE2 | GLN | A | 418 | 0.598 | 17.661 | 56.531 | 1.00 | 31.50 | N |
| ATOM | 3080 | C | GLN | A | 418 | 2.567 | 19.763 | 61.950 | 1.00 | 20.44 | C |
| ATOM | 3081 | O | GLN | A | 418 | 1.670 | 19.755 | 62.769 | 1.00 | 20.10 | O |
| ATOM | 3090 | N | LEU | A | 419 | 3.824 | 19.461 | 62.252 | 1.00 | 20.18 | N |
| ATOM | 3091 | CA | LEU | A | 419 | 4.250 | 19.159 | 63.605 | 1.00 | 20.32 | C |
| ATOM | 3092 | CB | LEU | A | 419 | 5.690 | 18.617 | 63.627 | 1.00 | 19.76 | C |
| ATOM | 3093 | CG | LEU | A | 419 | 5.892 | 17.220 | 63.023 | 1.00 | 19.28 | C |
| ATOM | 3094 | CD1 | LEU | A | 419 | 7.355 | 16.823 | 63.058 | 1.00 | 18.25 | C |
| ATOM | 3095 | CD2 | LEU | A | 419 | 5.045 | 16.175 | 63.730 | 1.00 | 20.07 | C |
| ATOM | 3096 | C | LEU | A | 419 | 4.130 | 20.365 | 64.539 | 1.00 | 20.91 | C |
| ATOM | 3097 | O | LEU | A | 419 | 3.780 | 20.201 | 65.693 | 1.00 | 20.09 | O |
| ATOM | 3109 | N | LEU | A | 420 | 4.428 | 21.562 | 64.037 | 1.00 | 21.53 | N |
| ATOM | 3110 | CA | LEU | A | 420 | 4.214 | 22.780 | 64.812 | 1.00 | 22.24 | C |
| ATOM | 3111 | CB | LEU | A | 420 | 4.778 | 24.005 | 64.088 | 1.00 | 22.10 | C |
| ATOM | 3112 | CG | LEU | A | 420 | 6.295 | 24.048 | 63.918 | 1.00 | 23.12 | C |
| ATOM | 3113 | CD1 | LEU | A | 420 | 6.739 | 25.397 | 63.360 | 1.00 | 24.11 | C |
| ATOM | 3114 | CD2 | LEU | A | 420 | 7.028 | 23.743 | 65.231 | 1.00 | 23.78 | C |
| ATOM | 3115 | C | LEU | A | 420 | 2.731 | 22.988 | 65.133 | 1.00 | 22.74 | C |
| ATOM | 3116 | O | LEU | A | 420 | 2.385 | 23.408 | 66.239 | 1.00 | 22.80 | O |
| ATOM | 3128 | N | LEU | A | 421 | 1.867 | 22.664 | 64.182 | 1.00 | 22.92 | N |
| ATOM | 3129 | CA | LEU | A | 421 | 0.439 | 22.745 | 64.400 | 1.00 | 23.62 | C |
| ATOM | 3130 | CB | LEU | A | 421 | −0.277 | 22.410 | 63.106 | 1.00 | 24.23 | C |
| ATOM | 3131 | CG | LEU | A | 421 | −1.434 | 23.291 | 62.674 | 1.00 | 25.57 | C |
| ATOM | 3132 | CD1 | LEU | A | 421 | −1.065 | 24.775 | 62.729 | 1.00 | 26.71 | C |
| ATOM | 3133 | CD2 | LEU | A | 421 | −1.814 | 22.881 | 61.260 | 1.00 | 25.87 | C |
| ATOM | 3134 | C | LEU | A | 421 | −0.018 | 21.775 | 65.491 | 1.00 | 23.45 | C |
| ATOM | 3135 | O | LEU | A | 421 | −0.964 | 22.048 | 66.229 | 1.00 | 23.05 | O |
| ATOM | 3147 | N | CYS | A | 422 | 0.660 | 20.631 | 65.557 | 1.00 | 23.26 | N |
| ATOM | 3148 | CA | CYS | A | 422 | 0.427 | 19.637 | 66.573 | 1.00 | 23.18 | C |
| ATOM | 3149 | CB | CYS | A | 422 | 1.278 | 18.412 | 66.323 | 1.00 | 23.22 | C |
| ATOM | 3150 | SG | CYS | A | 422 | 0.586 | 17.316 | 65.109 | 1.00 | 26.71 | S |
| ATOM | 3151 | C | CYS | A | 422 | 0.789 | 20.136 | 67.942 | 1.00 | 22.63 | C |
| ATOM | 3152 | O | CYS | A | 422 | 0.133 | 19.789 | 68.909 | 1.00 | 21.99 | O |
| ATOM | 3158 | N | LEU | A | 423 | 1.874 | 20.888 | 68.025 | 1.00 | 22.02 | N |
| ATOM | 3159 | CA | LEU | A | 423 | 2.281 | 21.498 | 69.274 | 1.00 | 22.11 | C |
| ATOM | 3160 | CB | LEU | A | 423 | 3.644 | 22.167 | 69.114 | 1.00 | 22.19 | C |
| ATOM | 3161 | CG | LEU | A | 423 | 4.857 | 21.403 | 69.682 | 1.00 | 22.64 | C |
| ATOM | 3162 | CD1 | LEU | A | 423 | 4.761 | 19.886 | 69.585 | 1.00 | 23.66 | C |
| ATOM | 3163 | CD2 | LEU | A | 423 | 6.163 | 21.851 | 69.082 | 1.00 | 23.15 | C |
| ATOM | 3164 | C | LEU | A | 423 | 1.234 | 22.489 | 69.788 | 1.00 | 22.00 | C |
| ATOM | 3165 | O | LEU | A | 423 | 1.042 | 22.613 | 70.974 | 1.00 | 22.66 | O |
| ATOM | 3177 | N | VAL | A | 424 | 0.544 | 23.166 | 68.889 | 1.00 | 21.95 | N |
| ATOM | 3178 | CA | VAL | A | 424 | −0.517 | 24.076 | 69.261 | 1.00 | 22.08 | C |
| ATOM | 3179 | CB | VAL | A | 424 | −1.028 | 24.876 | 68.045 | 1.00 | 22.12 | C |
| ATOM | 3180 | CG1 | VAL | A | 424 | −2.294 | 25.649 | 68.356 | 1.00 | 22.02 | C |
| ATOM | 3181 | CG2 | VAL | A | 424 | 0.056 | 25.860 | 67.549 | 1.00 | 23.78 | C |
| ATOM | 3182 | C | VAL | A | 424 | −1.618 | 23.282 | 69.922 | 1.00 | 21.76 | C |
| ATOM | 3183 | O | VAL | A | 424 | −2.159 | 23.692 | 70.948 | 1.00 | 22.10 | O |
| ATOM | 3193 | N | GLU | A | 425 | −1.909 | 22.119 | 69.368 | 1.00 | 21.82 | N |
| ATOM | 3194 | CA | GLU | A | 425 | −2.895 | 21.219 | 69.947 | 1.00 | 21.50 | C |
| ATOM | 3195 | CB | GLU | A | 425 | −3.188 | 20.096 | 68.971 | 1.00 | 21.50 | C |
| ATOM | 3196 | CG | GLU | A | 425 | −4.113 | 19.040 | 69.536 | 1.00 | 23.59 | C |
| ATOM | 3197 | CD | GLU | A | 425 | −5.475 | 19.597 | 69.907 | 1.00 | 25.13 | C |
| ATOM | 3198 | OE1 | GLU | A | 425 | −5.775 | 20.738 | 69.496 | 1.00 | 29.93 | O |
| ATOM | 3199 | OE2 | GLU | A | 425 | −6.242 | 18.899 | 70.583 | 1.00 | 24.97 | O |
| ATOM | 3200 | C | GLU | A | 425 | −2.470 | 20.626 | 71.281 | 1.00 | 21.37 | C |
| ATOM | 3201 | O | GLU | A | 425 | −3.279 | 20.419 | 72.139 | 1.00 | 20.52 | O |
| ATOM | 3208 | N | VAL | A | 426 | −1.189 | 20.342 | 71.452 | 1.00 | 21.91 | N |
| ATOM | 3209 | CA | VAL | A | 426 | −0.667 | 19.846 | 72.709 | 1.00 | 22.12 | C |
| ATOM | 3210 | CB | VAL | A | 426 | 0.841 | 19.537 | 72.617 | 1.00 | 22.40 | C |
| ATOM | 3211 | CG1 | VAL | A | 426 | 1.500 | 19.561 | 73.995 | 1.00 | 22.16 | C |
| ATOM | 3212 | CG2 | VAL | A | 426 | 1.074 | 18.220 | 71.935 | 1.00 | 23.67 | C |
| ATOM | 3213 | C | VAL | A | 426 | −0.870 | 20.905 | 73.782 | 1.00 | 22.38 | C |
| ATOM | 3214 | O | VAL | A | 426 | −1.193 | 20.592 | 74.908 | 1.00 | 22.52 | O |
| ATOM | 3224 | N | ARG | A | 427 | −0.673 | 22.156 | 73.428 | 1.00 | 22.52 | N |
| ATOM | 3225 | CA | ARG | A | 427 | −0.875 | 23.225 | 74.370 | 1.00 | 23.14 | C |
| ATOM | 3226 | CB | ARG | A | 427 | −0.296 | 24.502 | 73.761 | 1.00 | 23.80 | C |
| ATOM | 3227 | CG | ARG | A | 427 | −0.552 | 25.723 | 74.591 | 1.00 | 28.07 | C |
| ATOM | 3228 | CD | ARG | A | 427 | 0.366 | 26.946 | 74.314 | 1.00 | 32.33 | C |
| ATOM | 3229 | NE | ARG | A | 427 | 0.073 | 28.002 | 75.306 | 1.00 | 35.62 | N |
| ATOM | 3230 | CZ | ARG | A | 427 | −0.951 | 28.900 | 75.256 | 1.00 | 37.93 | C |
| ATOM | 3231 | NH1 | ARG | A | 427 | −1.814 | 28.958 | 74.226 | 1.00 | 36.94 | N |
| ATOM | 3232 | NH2 | ARG | A | 427 | −1.089 | 29.784 | 76.257 | 1.00 | 38.53 | N |
| ATOM | 3233 | C | ARG | A | 427 | −2.377 | 23.383 | 74.773 | 1.00 | 22.48 | C |
| ATOM | 3234 | O | ARG | A | 427 | −2.721 | 23.672 | 75.931 | 1.00 | 22.08 | O |
| ATOM | 3248 | N | ALA | A | 428 | −3.268 | 23.164 | 73.823 | 1.00 | 22.07 | N |
| ATOM | 3249 | CA | ALA | A | 428 | −4.685 | 23.348 | 74.056 | 1.00 | 22.12 | C |
| ATOM | 3250 | CB | ALA | A | 428 | −5.449 | 23.406 | 72.741 | 1.00 | 22.06 | C |
| ATOM | 3251 | C | ALA | A | 428 | −5.200 | 22.226 | 74.926 | 1.00 | 22.29 | C |
| ATOM | 3252 | O | ALA | A | 428 | −5.998 | 22.458 | 75.828 | 1.00 | 22.78 | O |
| ATOM | 3258 | N | LEU | A | 429 | −4.714 | 21.024 | 74.663 | 1.00 | 22.14 | N |
| ATOM | 3259 | CA | LEU | A | 429 | −4.971 | 19.852 | 75.483 | 1.00 | 22.59 | C |
| ATOM | 3260 | CB | LEU | A | 429 | −4.249 | 18.666 | 74.881 | 1.00 | 22.98 | C |
| ATOM | 3261 | CG | LEU | A | 429 | −4.986 | 17.393 | 74.493 | 1.00 | 25.32 | C |
| ATOM | 3262 | CD1 | LEU | A | 429 | −6.415 | 17.583 | 74.080 | 1.00 | 26.29 | C |
| ATOM | 3263 | CD2 | LEU | A | 429 | −4.200 | 16.753 | 73.362 | 1.00 | 27.92 | C |
| ATOM | 3264 | C | LEU | A | 429 | −4.488 | 19.994 | 76.917 | 1.00 | 22.44 | C |
| ATOM | 3265 | O | LEU | A | 429 | −5.155 | 19.584 | 77.858 | 1.00 | 21.62 | O |
| ATOM | 3277 | N | SER | A | 430 | −3.307 | 20.564 | 77.078 | 1.00 | 22.14 | N |
| ATOM | 3278 | CA | SER | A | 430 | −2.726 | 20.707 | 78.393 | 1.00 | 22.01 | C |
| ATOM | 3279 | CB | SER | A | 430 | −1.227 | 20.976 | 78.282 | 1.00 | 22.24 | C |
| ATOM | 3280 | OG | SER | A | 430 | −0.971 | 22.351 | 78.119 | 1.00 | 25.47 | O |
| ATOM | 3281 | C | SER | A | 430 | −3.468 | 21.736 | 79.242 | 1.00 | 21.52 | C |
| ATOM | 3282 | O | SER | A | 430 | −3.521 | 21.610 | 80.448 | 1.00 | 21.05 | O |
| ATOM | 3288 | N | MET | A | 431 | −4.081 | 22.715 | 78.595 | 1.00 | 21.64 | N |
| ATOM | 3289 | CA | MET | A | 431 | −4.979 | 23.654 | 79.236 | 1.00 | 21.88 | C |
| ATOM | 3290 | CB | MET | A | 431 | −5.324 | 24.752 | 78.236 | 1.00 | 22.82 | C |
| ATOM | 3291 | CG | MET | A | 431 | −5.955 | 25.980 | 78.843 | 1.00 | 26.42 | C |
| ATOM | 3292 | SD | MET | A | 431 | −6.381 | 27.348 | 77.679 | 1.00 | 34.08 | S |
| ATOM | 3293 | CE | MET | A | 431 | −8.031 | 27.647 | 78.149 | 1.00 | 31.99 | C |
| ATOM | 3294 | C | MET | A | 431 | −6.270 | 22.969 | 79.758 | 1.00 | 21.17 | C |
| ATOM | 3295 | O | MET | A | 431 | −6.660 | 23.168 | 80.902 | 1.00 | 19.28 | O |
| ATOM | 3305 | N | GLN | A | 432 | −6.902 | 22.148 | 78.920 | 1.00 | 21.13 | N |
| ATOM | 3306 | CA | GLN | A | 432 | −8.015 | 21.310 | 79.350 | 1.00 | 21.64 | C |
| ATOM | 3307 | CB | GLN | A | 432 | −8.595 | 20.439 | 78.232 | 1.00 | 21.71 | C |
| ATOM | 3308 | CG | GLN | A | 432 | −8.838 | 21.141 | 76.918 | 1.00 | 25.51 | C |
| ATOM | 3309 | CD | GLN | A | 432 | −10.029 | 20.584 | 76.163 | 1.00 | 27.85 | C |
| ATOM | 3310 | OE1 | GLN | A | 432 | −10.183 | 19.365 | 76.011 | 1.00 | 30.10 | O |
| ATOM | 3311 | NE2 | GLN | A | 432 | −10.890 | 21.479 | 75.708 | 1.00 | 30.95 | N |
| ATOM | 3312 | C | GLN | A | 432 | −7.605 | 20.390 | 80.480 | 1.00 | 20.60 | C |
| ATOM | 3313 | O | GLN | A | 432 | −8.407 | 20.099 | 81.324 | 1.00 | 21.14 | O |
| ATOM | 3322 | N | ALA | A | 433 | −6.366 | 19.937 | 80.491 | 1.00 | 20.19 | N |
| ATOM | 3323 | CA | ALA | A | 433 | −5.885 | 19.056 | 81.543 | 1.00 | 20.11 | C |
| ATOM | 3324 | CB | ALA | A | 433 | −4.532 | 18.390 | 81.137 | 1.00 | 19.80 | C |
| ATOM | 3325 | C | ALA | A | 433 | −5.780 | 19.744 | 82.902 | 1.00 | 19.69 | C |
| ATOM | 3326 | O | ALA | A | 433 | −6.185 | 19.174 | 83.914 | 1.00 | 19.36 | O |
| ATOM | 3332 | N | LYS | A | 434 | −5.228 | 20.956 | 82.918 | 1.00 | 19.12 | N |
| ATOM | 3333 | CA | LYS | A | 434 | −5.181 | 21.774 | 84.134 | 1.00 | 19.41 | C |
| ATOM | 3334 | CB | LYS | A | 434 | −4.479 | 23.081 | 83.847 | 1.00 | 19.36 | C |
| ATOM | 3335 | CG | LYS | A | 434 | −3.001 | 22.980 | 83.614 | 1.00 | 21.64 | C |
| ATOM | 3336 | CD | LYS | A | 434 | −2.527 | 24.334 | 83.135 | 1.00 | 22.59 | C |
| ATOM | 3337 | CE | LYS | A | 434 | −1.098 | 24.624 | 83.379 | 1.00 | 23.44 | C |
| ATOM | 3338 | NZ | LYS | A | 434 | −1.001 | 26.090 | 83.688 | 1.00 | 25.51 | N |
| ATOM | 3339 | C | LYS | A | 434 | −6.564 | 22.120 | 84.696 | 1.00 | 18.85 | C |
| ATOM | 3340 | O | LYS | A | 434 | −6.726 | 22.257 | 85.877 | 1.00 | 19.53 | O |
| ATOM | 3354 | N | GLU | A | 435 | −7.539 | 22.291 | 83.820 | 1.00 | 18.64 | N |
| ATOM | 3355 | CA | GLU | A | 435 | −8.895 | 22.591 | 84.195 | 1.00 | 18.22 | C |
| ATOM | 3356 | CB | GLU | A | 435 | −9.656 | 23.099 | 82.954 | 1.00 | 18.24 | C |
| ATOM | 3357 | CG | GLU | A | 435 | −9.142 | 24.445 | 82.447 | 1.00 | 19.28 | C |
| ATOM | 3358 | CD | GLU | A | 435 | −9.757 | 24.916 | 81.127 | 1.00 | 21.29 | C |
| ATOM | 3359 | OE1 | GLU | A | 435 | −9.426 | 26.023 | 80.682 | 1.00 | 22.03 | O |
| ATOM | 3360 | OE2 | GLU | A | 435 | −10.584 | 24.221 | 80.524 | 1.00 | 24.43 | O |
| ATOM | 3361 | C | GLU | A | 435 | −9.568 | 21.361 | 84.806 | 1.00 | 18.08 | C |
| ATOM | 3362 | O | GLU | A | 435 | −10.323 | 21.482 | 85.771 | 1.00 | 17.60 | O |
| ATOM | 3369 | N | TYR | A | 436 | −9.280 | 20.178 | 84.263 | 1.00 | 18.46 | N |
| ATOM | 3370 | CA | TYR | A | 436 | −9.705 | 18.909 | 84.871 | 1.00 | 18.96 | C |
| ATOM | 3371 | CB | TYR | A | 436 | −9.261 | 17.696 | 84.046 | 1.00 | 19.26 | C |
| ATOM | 3372 | CG | TYR | A | 436 | −9.386 | 16.356 | 84.793 | 1.00 | 20.38 | C |
| ATOM | 3373 | CD1 | TYR | A | 436 | −10.632 | 15.718 | 84.961 | 1.00 | 19.35 | C |
| ATOM | 3374 | CE1 | TYR | A | 436 | −10.735 | 14.496 | 85.654 | 1.00 | 18.63 | C |
| ATOM | 3375 | CZ | TYR | A | 436 | −9.596 | 13.911 | 86.191 | 1.00 | 18.69 | C |
| ATOM | 3376 | OH | TYR | A | 436 | −9.657 | 12.721 | 86.877 | 1.00 | 17.66 | O |
| ATOM | 3377 | CE2 | TYR | A | 436 | −8.367 | 14.518 | 86.035 | 1.00 | 20.19 | C |
| ATOM | 3378 | CD2 | TYR | A | 436 | −8.263 | 15.731 | 85.337 | 1.00 | 20.27 | C |
| ATOM | 3379 | C | TYR | A | 436 | −9.118 | 18.787 | 86.249 | 1.00 | 19.34 | C |
| ATOM | 3380 | O | TYR | A | 436 | −9.828 | 18.535 | 87.194 | 1.00 | 19.29 | O |
| ATOM | 3390 | N | LEU | A | 437 | −7.810 | 18.996 | 86.349 | 1.00 | 19.64 | N |
| ATOM | 3391 | CA | LEU | A | 437 | −7.112 | 19.060 | 87.635 | 1.00 | 19.87 | C |
| ATOM | 3392 | CB | LEU | A | 437 | −5.657 | 19.463 | 87.424 | 1.00 | 20.12 | C |
| ATOM | 3393 | CG | LEU | A | 437 | −4.533 | 18.431 | 87.561 | 1.00 | 22.65 | C |
| ATOM | 3394 | CD1 | LEU | A | 437 | −4.965 | 16.968 | 87.674 | 1.00 | 24.13 | C |
| ATOM | 3395 | CD2 | LEU | A | 437 | −3.523 | 18.605 | 86.433 | 1.00 | 23.71 | C |
| ATOM | 3396 | C | LEU | A | 437 | −7.716 | 20.034 | 88.626 | 1.00 | 18.85 | C |
| ATOM | 3397 | O | LEU | A | 437 | −7.891 | 19.702 | 89.782 | 1.00 | 18.16 | O |
| ATOM | 3409 | N | TYR | A | 438 | −8.021 | 21.236 | 88.170 | 1.00 | 17.93 | N |
| ATOM | 3410 | CA | TYR | A | 438 | −8.580 | 22.263 | 89.034 | 1.00 | 17.38 | C |
| ATOM | 3411 | CB | TYR | A | 438 | −8.583 | 23.581 | 88.280 | 1.00 | 16.90 | C |
| ATOM | 3412 | CG | TYR | A | 438 | −9.069 | 24.767 | 89.069 | 1.00 | 17.24 | C |
| ATOM | 3413 | CD1 | TYR | A | 438 | −8.266 | 25.360 | 90.044 | 1.00 | 17.55 | C |
| ATOM | 3414 | CE1 | TYR | A | 438 | −8.702 | 26.440 | 90.776 | 1.00 | 15.15 | C |
| ATOM | 3415 | CZ | TYR | A | 438 | −9.936 | 26.981 | 90.518 | 1.00 | 16.02 | C |
| ATOM | 3416 | OH | TYR | A | 438 | −10.363 | 28.067 | 91.233 | 1.00 | 14.28 | O |
| ATOM | 3417 | CE2 | TYR | A | 438 | −10.763 | 26.418 | 89.551 | 1.00 | 16.67 | C |
| ATOM | 3418 | CD2 | TYR | A | 438 | −10.329 | 25.320 | 88.833 | 1.00 | 16.48 | C |
| ATOM | 3419 | C | TYR | A | 438 | −9.988 | 21.886 | 89.535 | 1.00 | 16.97 | C |
| ATOM | 3420 | O | TYR | A | 438 | −10.349 | 22.138 | 90.680 | 1.00 | 16.23 | O |
| ATOM | 3430 | N | HIS | A | 439 | −10.764 | 21.256 | 88.663 | 1.00 | 16.84 | N |
| ATOM | 3431 | CA | HIS | A | 439 | −12.044 | 20.697 | 89.023 | 1.00 | 16.24 | C |
| ATOM | 3432 | CB | HIS | A | 439 | −12.662 | 20.075 | 87.787 | 1.00 | 16.41 | C |
| ATOM | 3433 | CG | HIS | A | 439 | −13.940 | 19.362 | 88.050 | 1.00 | 17.16 | C |
| ATOM | 3434 | ND1 | HIS | A | 439 | −15.089 | 20.019 | 88.429 | 1.00 | 19.56 | N |
| ATOM | 3435 | CE1 | HIS | A | 439 | −16.053 | 19.135 | 88.620 | 1.00 | 18.80 | C |
| ATOM | 3436 | NE2 | HIS | A | 439 | −15.561 | 17.931 | 88.393 | 1.00 | 18.39 | N |
| ATOM | 3437 | CD2 | HIS | A | 439 | −14.241 | 18.043 | 88.041 | 1.00 | 17.24 | C |
| ATOM | 3438 | C | HIS | A | 439 | −11.922 | 19.687 | 90.167 | 1.00 | 15.77 | C |
| ATOM | 3439 | O | HIS | A | 439 | −12.604 | 19.810 | 91.163 | 1.00 | 15.02 | O |
| ATOM | 3448 | N | LYS | A | 440 | −11.017 | 18.719 | 90.045 | 1.00 | 16.25 | N |
| ATOM | 3449 | CA | LYS | A | 440 | −10.769 | 17.713 | 91.099 | 1.00 | 16.49 | C |
| ATOM | 3450 | CB | LYS | A | 440 | −9.698 | 16.704 | 90.683 | 1.00 | 16.38 | C |
| ATOM | 3451 | CG | LYS | A | 440 | −9.955 | 15.960 | 89.382 | 1.00 | 17.80 | C |
| ATOM | 3452 | CD | LYS | A | 440 | −11.308 | 15.227 | 89.324 | 1.00 | 19.43 | C |
| ATOM | 3453 | CE | LYS | A | 440 | −11.317 | 13.973 | 90.160 | 1.00 | 20.78 | C |
| ATOM | 3454 | NZ | LYS | A | 440 | −12.522 | 13.172 | 89.858 | 1.00 | 21.15 | N |
| ATOM | 3455 | C | LYS | A | 440 | −10.346 | 18.333 | 92.420 | 1.00 | 16.31 | C |
| ATOM | 3456 | O | LYS | A | 440 | −10.779 | 17.919 | 93.479 | 1.00 | 15.33 | O |
| ATOM | 3470 | N | HIS | A | 441 | −9.511 | 19.357 | 92.320 | 1.00 | 17.08 | N |
| ATOM | 3471 | CA | HIS | A | 441 | −8.972 | 20.066 | 93.458 | 1.00 | 17.11 | C |
| ATOM | 3472 | CB | HIS | A | 441 | −7.863 | 21.040 | 92.981 | 1.00 | 17.64 | C |
| ATOM | 3473 | CG | HIS | A | 441 | −7.518 | 22.103 | 93.982 | 1.00 | 18.38 | C |
| ATOM | 3474 | ND1 | HIS | A | 441 | −6.758 | 21.852 | 95.104 | 1.00 | 20.55 | N |
| ATOM | 3475 | CE1 | HIS | A | 441 | −6.652 | 22.957 | 95.820 | 1.00 | 21.85 | C |
| ATOM | 3476 | NE2 | HIS | A | 441 | −7.332 | 23.912 | 95.208 | 1.00 | 22.54 | N |
| ATOM | 3477 | CD2 | HIS | A | 441 | −7.887 | 23.401 | 94.060 | 1.00 | 19.63 | C |
| ATOM | 3478 | C | HIS | A | 441 | −10.057 | 20.796 | 94.244 | 1.00 | 16.93 | C |
| ATOM | 3479 | O | HIS | A | 441 | −10.047 | 20.771 | 95.462 | 1.00 | 17.41 | O |
| ATOM | 3488 | N | LEU | A | 442 | −10.984 | 21.445 | 93.551 | 1.00 | 17.18 | N |
| ATOM | 3489 | CA | LEU | A | 442 | −12.047 | 22.185 | 94.210 | 1.00 | 17.67 | C |
| ATOM | 3490 | CB | LEU | A | 442 | −12.800 | 23.055 | 93.209 | 1.00 | 17.57 | C |
| ATOM | 3491 | CG | LEU | A | 442 | −12.105 | 24.319 | 92.690 | 1.00 | 18.90 | C |
| ATOM | 3492 | CD1 | LEU | A | 442 | −13.127 | 25.131 | 91.904 | 1.00 | 20.19 | C |
| ATOM | 3493 | CD2 | LEU | A | 442 | −11.471 | 25.184 | 93.785 | 1.00 | 18.70 | C |
| ATOM | 3494 | C | LEU | A | 442 | −13.028 | 21.257 | 94.949 | 1.00 | 17.99 | C |
| ATOM | 3495 | O | LEU | A | 442 | −13.580 | 21.631 | 95.958 | 1.00 | 17.24 | O |
| ATOM | 3507 | N | GLY | A | 443 | −13.213 | 20.046 | 94.439 | 1.00 | 19.06 | N |
| ATOM | 3508 | CA | GLY | A | 443 | −14.031 | 19.043 | 95.083 | 1.00 | 19.99 | C |
| ATOM | 3509 | C | GLY | A | 443 | −13.265 | 18.152 | 96.048 | 1.00 | 21.13 | C |
| ATOM | 3510 | O | GLY | A | 443 | −13.752 | 17.092 | 96.432 | 1.00 | 21.16 | O |
| ATOM | 3514 | N | ASN | A | 444 | −12.075 | 18.590 | 96.448 | 1.00 | 22.73 | N |
| ATOM | 3515 | CA | ASN | A | 444 | −11.206 | 17.844 | 97.365 | 1.00 | 23.90 | C |
| ATOM | 3516 | CB | ASN | A | 444 | −11.714 | 17.995 | 98.812 | 1.00 | 23.88 | C |
| ATOM | 3517 | CG | ASN | A | 444 | −10.582 | 18.034 | 99.831 | 1.00 | 26.27 | C |
| ATOM | 3518 | OD1 | ASN | A | 444 | −9.478 | 18.485 | 99.522 | 1.00 | 32.29 | O |
| ATOM | 3519 | ND2 | ASN | A | 444 | −10.847 | 17.567 | 101.051 | 1.00 | 27.48 | N |
| ATOM | 3520 | C | ASN | A | 444 | −10.997 | 16.350 | 97.001 | 1.00 | 24.01 | C |
| ATOM | 3521 | O | ASN | A | 444 | −10.868 | 15.509 | 97.888 | 1.00 | 24.08 | O |
| ATOM | 3528 | N | GLU | A | 445 | −10.930 | 16.051 | 95.700 | 1.00 | 24.16 | N |
| ATOM | 3529 | CA | GLU | A | 445 | −10.796 | 14.670 | 95.184 | 1.00 | 24.21 | C |
| ATOM | 3530 | CB | GLU | A | 445 | −11.645 | 14.506 | 93.913 | 1.00 | 23.91 | C |
| ATOM | 3531 | CG | GLU | A | 445 | −13.121 | 14.317 | 94.233 | 1.00 | 25.54 | C |
| ATOM | 3532 | CD | GLU | A | 445 | −14.082 | 14.870 | 93.194 | 1.00 | 26.62 | C |
| ATOM | 3533 | OE1 | GLU | A | 445 | −15.297 | 14.945 | 93.494 | 1.00 | 30.08 | O |
| ATOM | 3534 | OE2 | GLU | A | 445 | −13.654 | 15.226 | 92.087 | 1.00 | 28.63 | O |
| ATOM | 3535 | C | GLU | A | 445 | −9.342 | 14.207 | 94.918 | 1.00 | 23.92 | C |
| ATOM | 3536 | O | GLU | A | 445 | −9.100 | 13.029 | 94.664 | 1.00 | 23.88 | O |
| ATOM | 3543 | N | MET | A | 446 | −8.388 | 15.127 | 94.982 | 1.00 | 23.69 | N |
| ATOM | 3544 | CA | MET | A | 446 | −6.992 | 14.791 | 94.740 | 1.00 | 23.62 | C |
| ATOM | 3545 | CB | MET | A | 446 | −6.250 | 15.976 | 94.099 | 1.00 | 23.35 | C |
| ATOM | 3546 | CG | MET | A | 446 | −6.784 | 16.449 | 92.771 | 1.00 | 22.16 | C |
| ATOM | 3547 | SD | MET | A | 446 | −6.763 | 15.187 | 91.490 | 1.00 | 22.14 | S |
| ATOM | 3548 | CE | MET | A | 446 | −5.016 | 15.109 | 91.079 | 1.00 | 21.86 | C |
| ATOM | 3549 | C | MET | A | 446 | −6.270 | 14.396 | 96.024 | 1.00 | 23.74 | C |
| ATOM | 3550 | O | MET | A | 446 | −6.569 | 14.912 | 97.092 | 1.00 | 24.01 | O |
| ATOM | 3560 | N | PRO | A | 447 | −5.286 | 13.504 | 95.918 | 1.00 | 24.34 | N |
| ATOM | 3561 | CA | PRO | A | 447 | −4.416 | 13.186 | 97.055 | 1.00 | 24.52 | C |
| ATOM | 3562 | CB | PRO | A | 447 | −3.321 | 12.329 | 96.427 | 1.00 | 24.38 | C |
| ATOM | 3563 | CG | PRO | A | 447 | −3.890 | 11.778 | 95.221 | 1.00 | 23.68 | C |
| ATOM | 3564 | CD | PRO | A | 447 | −4.917 | 12.732 | 94.718 | 1.00 | 24.29 | C |
| ATOM | 3565 | C | PRO | A | 447 | −3.820 | 14.452 | 97.652 | 1.00 | 25.05 | C |
| ATOM | 3566 | O | PRO | A | 447 | −3.561 | 15.408 | 96.926 | 1.00 | 25.52 | O |
| ATOM | 3574 | N | PRO | A | 448 | −3.608 | 14.445 | 98.957 | 1.00 | 25.61 | N |
| ATOM | 3575 | CA | PRO | A | 448 | −3.270 | 15.660 | 99.707 | 1.00 | 26.15 | C |
| ATOM | 3576 | CB | ARG | A | 448 | −3.287 | 15.374 | 101.212 | 1.00 | 26.49 | C |
| ATOM | 3577 | CG | ARG | A | 448 | −4.133 | 16.340 | 102.041 | 1.00 | 29.42 | C |
| ATOM | 3578 | CD | ARG | A | 448 | −4.001 | 16.153 | 103.587 | 1.00 | 31.87 | C |
| ATOM | 3579 | NE | ARG | A | 448 | −2.591 | 16.166 | 104.016 | 1.00 | 34.77 | N |
| ATOM | 3580 | CZ | ARG | A | 448 | −1.777 | 15.097 | 104.058 | 1.00 | 35.53 | C |
| ATOM | 3581 | NH1 | ARG | A | 448 | −2.225 | 13.886 | 103.705 | 1.00 | 36.15 | N |
| ATOM | 3582 | NH2 | ARG | A | 448 | −0.509 | 15.242 | 104.458 | 1.00 | 34.42 | N |
| ATOM | 3583 | C | ARG | A | 448 | −1.911 | 16.240 | 99.347 | 1.00 | 25.78 | C |
| ATOM | 3584 | O | ARG | A | 448 | −1.657 | 17.427 | 99.570 | 1.00 | 26.34 | O |
| ATOM | 3598 | N | ASN | A | 449 | −1.012 | 15.418 | 98.842 | 1.00 | 24.73 | N |
| ATOM | 3599 | CA | ASN | A | 449 | 0.298 | 15.938 | 98.574 | 1.00 | 24.59 | C |
| ATOM | 3600 | CB | ASN | A | 449 | 1.287 | 15.326 | 99.569 | 1.00 | 25.27 | C |
| ATOM | 3601 | CG | ASN | A | 449 | 1.389 | 16.153 | 100.868 | 1.00 | 28.02 | C |
| ATOM | 3602 | OD1 | ASN | A | 449 | 1.002 | 15.705 | 101.965 | 1.00 | 29.49 | O |
| ATOM | 3603 | ND2 | ASN | A | 449 | 1.901 | 17.383 | 100.737 | 1.00 | 31.03 | N |
| ATOM | 3604 | C | ASN | A | 449 | 0.688 | 15.756 | 97.102 | 1.00 | 23.59 | C |
| ATOM | 3605 | O | ASN | A | 449 | 1.870 | 15.652 | 96.771 | 1.00 | 23.24 | O |
| ATOM | 3612 | N | ASN | A | 450 | −0.334 | 15.800 | 96.235 | 1.00 | 21.88 | N |
| ATOM | 3613 | CA | ASN | A | 450 | −0.176 | 15.680 | 94.806 | 1.00 | 21.08 | C |
| ATOM | 3614 | CB | ASN | A | 450 | −1.547 | 15.744 | 94.142 | 1.00 | 21.25 | C |
| ATOM | 3615 | CG | ASN | A | 450 | −1.516 | 15.421 | 92.690 | 1.00 | 19.62 | C |
| ATOM | 3616 | OD1 | ASN | A | 450 | −1.457 | 16.302 | 91.863 | 1.00 | 18.95 | O |
| ATOM | 3617 | ND2 | ASN | A | 450 | −1.593 | 14.145 | 92.364 | 1.00 | 22.27 | N |
| ATOM | 3618 | C | ASN | A | 450 | 0.741 | 16.770 | 94.252 | 1.00 | 20.62 | C |
| ATOM | 3619 | O | ASN | A | 450 | 0.613 | 17.951 | 94.593 | 1.00 | 20.11 | O |
| ATOM | 3626 | N | LEU | A | 451 | 1.679 | 16.343 | 93.406 | 1.00 | 19.70 | N |
| ATOM | 3627 | CA | LEU | A | 451 | 2.646 | 17.234 | 92.800 | 1.00 | 18.92 | C |
| ATOM | 3628 | CB | LEU | A | 451 | 3.845 | 16.442 | 92.281 | 1.00 | 18.93 | C |
| ATOM | 3629 | CG | LEU | A | 451 | 4.580 | 15.564 | 93.311 | 1.00 | 18.03 | C |
| ATOM | 3630 | CD1 | LEU | A | 451 | 5.864 | 15.065 | 92.722 | 1.00 | 16.94 | C |
| ATOM | 3631 | CD2 | LEU | A | 451 | 4.867 | 16.251 | 94.646 | 1.00 | 18.18 | C |
| ATOM | 3632 | C | LEU | A | 451 | 2.051 | 18.075 | 91.691 | 1.00 | 18.24 | C |
| ATOM | 3633 | O | LEU | A | 451 | 2.413 | 19.237 | 91.535 | 1.00 | 17.33 | O |
| ATOM | 3645 | N | LEU | A | 452 | 1.120 | 17.512 | 90.934 | 1.00 | 18.42 | N |
| ATOM | 3646 | CA | LEU | A | 452 | 0.528 | 18.276 | 89.834 | 1.00 | 18.97 | C |
| ATOM | 3647 | CB | LEU | A | 452 | −0.249 | 17.387 | 88.850 | 1.00 | 19.13 | C |
| ATOM | 3648 | CG | LEU | A | 452 | 0.471 | 16.176 | 88.254 | 1.00 | 18.55 | C |
| ATOM | 3649 | CD1 | LEU | A | 452 | −0.357 | 15.616 | 87.148 | 1.00 | 18.89 | C |
| ATOM | 3650 | CD2 | LEU | A | 452 | 1.876 | 16.459 | 87.760 | 1.00 | 18.82 | C |
| ATOM | 3651 | C | LEU | A | 452 | −0.311 | 19.443 | 90.354 | 1.00 | 19.05 | C |
| ATOM | 3652 | O | LEU | A | 452 | −0.304 | 20.504 | 89.757 | 1.00 | 18.60 | O |
| ATOM | 3664 | N | ILE | A | 453 | −0.947 | 19.261 | 91.512 | 1.00 | 19.14 | N |
| ATOM | 3665 | CA | ILE | A | 453 | −1.729 | 20.321 | 92.136 | 1.00 | 19.46 | C |
| ATOM | 3666 | CB | ILE | A | 453 | −2.659 | 19.753 | 93.256 | 1.00 | 19.21 | C |
| ATOM | 3667 | CG1 | ILE | A | 453 | −3.702 | 18.795 | 92.671 | 1.00 | 19.29 | C |
| ATOM | 3668 | CD1 | ILE | A | 453 | −4.629 | 19.427 | 91.644 | 1.00 | 19.78 | C |
| ATOM | 3669 | CG2 | ILE | A | 453 | −3.325 | 20.875 | 94.028 | 1.00 | 19.64 | C |
| ATOM | 3670 | C | ILE | A | 453 | −0.816 | 21.411 | 92.678 | 1.00 | 19.12 | C |
| ATOM | 3671 | O | ILE | A | 453 | −1.128 | 22.573 | 92.588 | 1.00 | 18.36 | O |
| ATOM | 3683 | N | GLU | A | 454 | 0.307 | 21.024 | 93.248 | 1.00 | 19.27 | N |
| ATOM | 3684 | CA | GLU | A | 454 | 1.275 | 21.986 | 93.716 | 1.00 | 19.99 | C |
| ATOM | 3685 | CB | GLU | A | 454 | 2.379 | 21.275 | 94.504 | 1.00 | 20.28 | C |
| ATOM | 3686 | CG | GLU | A | 454 | 3.556 | 22.151 | 94.870 | 1.00 | 22.28 | C |
| ATOM | 3687 | CD | GLU | A | 454 | 4.412 | 21.584 | 96.006 | 1.00 | 24.40 | C |
| ATOM | 3688 | OE1 | GLU | A | 454 | 4.325 | 20.349 | 96.311 | 1.00 | 23.52 | O |
| ATOM | 3689 | OE2 | GLU | A | 454 | 5.174 | 22.406 | 96.581 | 1.00 | 24.50 | O |
| ATOM | 3690 | C | GLU | A | 454 | 1.838 | 22.802 | 92.567 | 1.00 | 20.15 | C |
| ATOM | 3691 | O | GLU | A | 454 | 2.115 | 23.970 | 92.750 | 1.00 | 20.41 | O |
| ATOM | 3698 | N | MET | A | 455 | 1.990 | 22.198 | 91.392 | 1.00 | 20.68 | N |
| ATOM | 3699 | CA | MET | A | 455 | 2.458 | 22.905 | 90.183 | 1.00 | 21.84 | C |
| ATOM | 3700 | CB | MET | A | 455 | 2.872 | 21.904 | 89.104 | 1.00 | 22.12 | C |
| ATOM | 3701 | CG | MET | A | 455 | 4.132 | 21.097 | 89.446 | 1.00 | 22.21 | C |
| ATOM | 3702 | SD | MET | A | 455 | 5.554 | 22.148 | 89.621 | 1.00 | 21.64 | S |
| ATOM | 3703 | CE | MET | A | 455 | 5.546 | 22.999 | 88.110 | 1.00 | 21.41 | C |
| ATOM | 3704 | C | MET | A | 455 | 1.422 | 23.857 | 89.588 | 1.00 | 22.47 | C |
| ATOM | 3705 | O | MET | A | 455 | 1.757 | 24.916 | 89.078 | 1.00 | 22.12 | O |
| ATOM | 3715 | N | LEU | A | 456 | 0.168 | 23.442 | 89.679 | 1.00 | 23.51 | N |
| ATOM | 3716 | CA | LEU | A | 456 | −0.994 | 24.202 | 89.255 | 1.00 | 24.84 | C |
| ATOM | 3717 | CB | LEU | A | 456 | −2.234 | 23.326 | 89.463 | 1.00 | 24.74 | C |
| ATOM | 3718 | CG | LEU | A | 456 | −3.514 | 23.598 | 88.687 | 1.00 | 25.88 | C |
| ATOM | 3719 | CD1 | LEU | A | 456 | −3.407 | 23.131 | 87.267 | 1.00 | 27.08 | C |
| ATOM | 3720 | CD2 | LEU | A | 456 | −4.675 | 22.887 | 89.366 | 1.00 | 27.47 | C |
| ATOM | 3721 | C | LEU | A | 456 | −1.129 | 25.499 | 90.057 | 1.00 | 26.00 | C |
| ATOM | 3722 | O | LEU | A | 456 | −1.456 | 26.557 | 89.510 | 1.00 | 25.65 | O |
| ATOM | 3734 | N | GLN | A | 457 | −0.831 | 25.421 | 91.349 | 1.00 | 27.59 | N |
| ATOM | 3735 | CA | GLN | A | 457 | −1.184 | 26.491 | 92.268 | 1.00 | 29.35 | C |
| ATOM | 3736 | CB | GLN | A | 457 | −1.459 | 25.970 | 93.691 | 1.00 | 29.84 | C |
| ATOM | 3737 | CG | GLN | A | 457 | −2.283 | 24.723 | 93.794 | 1.00 | 31.50 | C |
| ATOM | 3738 | CD | GLN | A | 457 | −3.759 | 24.992 | 93.792 | 1.00 | 34.74 | C |
| ATOM | 3739 | OE1 | GLN | A | 457 | −4.364 | 25.065 | 92.706 | 1.00 | 38.46 | O |
| ATOM | 3740 | NE2 | GLN | A | 457 | −4.369 | 25.123 | 94.992 | 1.00 | 34.45 | N |
| ATOM | 3741 | C | GLN | A | 457 | −0.109 | 27.564 | 92.367 | 1.00 | 29.98 | C |
| ATOM | 3742 | O | GLN | A | 457 | −0.322 | 28.566 | 93.064 | 1.00 | 29.59 | O |
| ATOM | 3751 | N | ALA | A | 458 | 1.025 | 27.351 | 91.701 | 1.00 | 31.10 | N |
| ATOM | 3752 | CA | ALA | A | 458 | 2.163 | 28.273 | 91.774 | 1.00 | 32.37 | C |
| ATOM | 3753 | CB | ALA | A | 458 | 3.445 | 27.483 | 92.026 | 1.00 | 32.18 | C |
| ATOM | 3754 | C | ALA | A | 458 | 2.328 | 29.165 | 90.523 | 1.00 | 33.97 | C |
| ATOM | 3755 | O | ALA | A | 458 | 3.175 | 28.880 | 89.668 | 1.00 | 34.67 | O |
| ATOM | 3761 | N | LYS | A | 459 | 1.508 | 30.220 | 90.447 | 1.00 | 35.40 | N |
| ATOM | 3762 | CA | LYS | A | 459 | 1.638 | 31.393 | 89.531 | 1.00 | 36.40 | C |
| ATOM | 3763 | CB | LYS | A | 459 | 2.193 | 32.609 | 90.292 | 1.00 | 36.16 | C |
| ATOM | 3764 | CG | LYS | A | 459 | 1.184 | 33.188 | 91.284 | 1.00 | 35.23 | C |
| ATOM | 3765 | CD | LYS | A | 459 | 1.743 | 34.410 | 92.062 | 1.00 | 34.96 | C |
| ATOM | 3766 | CE | LYS | A | 459 | 0.793 | 34.852 | 93.236 | 1.00 | 34.01 | C |
| ATOM | 3767 | NZ | LYS | A | 459 | 1.397 | 35.897 | 94.195 | 1.00 | 32.95 | N |
| ATOM | 3768 | C | LYS | A | 459 | 2.314 | 31.221 | 88.134 | 1.00 | 38.21 | C |
| ATOM | 3769 | O | LYS | A | 459 | 3.499 | 31.557 | 87.870 | 1.00 | 38.13 | O |
| ATOM | 3783 | N | GLN | A | 460 | 1.491 | 30.645 | 87.265 | 1.00 | 40.18 | N |
| ATOM | 3784 | CA | GLN | A | 460 | 1.552 | 30.840 | 85.829 | 1.00 | 41.41 | C |
| ATOM | 3785 | CB | GLN | A | 460 | 1.320 | 29.495 | 85.095 | 1.00 | 41.20 | C |
| ATOM | 3786 | CG | GLN | A | 460 | 2.033 | 28.276 | 85.723 | 1.00 | 41.20 | C |
| ATOM | 3787 | CD | GLN | A | 460 | 1.076 | 27.176 | 86.239 | 1.00 | 42.91 | C |
| ATOM | 3788 | OE1 | GLN | A | 460 | 1.596 | 25.977 | 86.390 | 1.00 | 46.44 | O |
| ATOM | 3789 | NE2 | GLN | A | 460 | −0.102 | 27.423 | 86.513 | 1.00 | 41.96 | N |
| ATOM | 3790 | C | GLN | A | 460 | 0.434 | 31.874 | 85.503 | 1.00 | 42.41 | C |
| ATOM | 3791 | O | GLN | A | 460 | −0.117 | 32.573 | 86.385 | 1.00 | 43.76 | O |
| ATOM | 3792 | OXT | GLN | A | 460 | −0.116 | 32.572 | 86.386 | 1.00 | 43.75 | O |
| ATOM | 3801 | N | PRO | B | 221 | −6.216 | −19.409 | 113.153 | 1.00 | 30.56 | N |
| ATOM | 3802 | CA | PRO | B | 221 | −5.628 | −19.881 | 114.445 | 1.00 | 30.36 | C |
| ATOM | 3803 | CB | PRO | B | 221 | −6.714 | −19.520 | 115.471 | 1.00 | 30.42 | C |
| ATOM | 3804 | CG | PRO | B | 221 | −7.330 | −18.245 | 114.917 | 1.00 | 30.92 | C |
| ATOM | 3805 | CD | PRO | B | 221 | −7.259 | −18.388 | 113.383 | 1.00 | 30.88 | C |
| ATOM | 3806 | C | PRO | B | 221 | −5.321 | −21.384 | 114.496 | 1.00 | 30.10 | C |
| ATOM | 3807 | O | PRO | B | 221 | −5.570 | −22.117 | 113.520 | 1.00 | 29.92 | O |
| ATOM | 3815 | N | ASN | B | 222 | −4.810 | −21.813 | 115.660 | 1.00 | 29.52 | N |
| ATOM | 3816 | CA | ASN | B | 222 | −4.258 | −23.148 | 115.863 | 1.00 | 28.81 | C |
| ATOM | 3817 | CB | ASN | B | 222 | −5.348 | −24.241 | 115.778 | 1.00 | 29.24 | C |
| ATOM | 3818 | CG | ASN | B | 222 | −6.781 | −23.684 | 115.837 | 1.00 | 30.75 | C |
| ATOM | 3819 | OD1 | ASN | B | 222 | −7.532 | −23.764 | 114.854 | 1.00 | 32.35 | O |
| ATOM | 3820 | ND2 | ASN | B | 222 | −7.169 | −23.141 | 116.996 | 1.00 | 32.77 | N |
| ATOM | 3821 | C | ASN | B | 222 | −3.126 | −23.396 | 114.851 | 1.00 | 27.38 | C |
| ATOM | 3822 | O | ASN | B | 222 | −3.180 | −24.345 | 114.062 | 1.00 | 27.55 | O |
| ATOM | 3829 | N | VAL | B | 223 | −2.129 | −22.508 | 114.848 | 1.00 | 25.83 | N |
| ATOM | 3830 | CA | VAL | B | 223 | −0.978 | −22.637 | 113.951 | 1.00 | 24.40 | C |
| ATOM | 3831 | CB | VAL | B | 223 | −0.911 | −21.547 | 112.851 | 1.00 | 24.21 | C |
| ATOM | 3832 | CG1 | VAL | B | 223 | 0.244 | −21.857 | 111.868 | 1.00 | 23.35 | C |
| ATOM | 3833 | CG2 | VAL | B | 223 | −2.225 | −21.427 | 112.085 | 1.00 | 24.00 | C |
| ATOM | 3834 | C | VAL | B | 223 | 0.293 | −22.553 | 114.767 | 1.00 | 23.64 | C |
| ATOM | 3835 | O | VAL | B | 223 | 0.526 | −21.542 | 115.413 | 1.00 | 23.55 | O |
| ATOM | 3845 | N | PRO | B | 224 | 1.124 | −23.597 | 114.728 | 1.00 | 22.92 | N |
| ATOM | 3846 | CA | PRO | B | 224 | 2.374 | −23.618 | 115.506 | 1.00 | 22.54 | C |
| ATOM | 3847 | CB | PRO | B | 224 | 3.022 | −24.955 | 115.115 | 1.00 | 22.12 | C |
| ATOM | 3848 | CG | PRO | B | 224 | 1.935 | −25.780 | 114.588 | 1.00 | 22.30 | C |
| ATOM | 3849 | CD | PRO | B | 224 | 0.934 | −24.850 | 113.975 | 1.00 | 22.61 | C |
| ATOM | 3850 | C | PRO | B | 224 | 3.317 | −22.458 | 115.202 | 1.00 | 22.24 | C |
| ATOM | 3851 | O | PRO | B | 224 | 3.530 | −22.119 | 114.052 | 1.00 | 21.75 | O |
| ATOM | 3859 | N | GLU | B | 225 | 3.876 | −21.873 | 116.247 | 1.00 | 22.61 | N |
| ATOM | 3860 | CA | GLU | B | 225 | 4.769 | −20.728 | 116.120 | 1.00 | 23.07 | C |
| ATOM | 3861 | CB | GLU | B | 225 | 5.391 | −20.366 | 117.492 | 1.00 | 23.28 | C |
| ATOM | 3862 | CG | GLU | B | 225 | 6.430 | −19.216 | 117.523 | 1.00 | 24.29 | C |
| ATOM | 3863 | CD | GLU | B | 225 | 5.929 | −17.828 | 117.073 | 1.00 | 26.09 | C |
| ATOM | 3864 | OE1 | GLU | B | 225 | 4.736 | −17.662 | 116.718 | 1.00 | 26.54 | O |
| ATOM | 3865 | OE2 | GLU | B | 225 | 6.755 | −16.873 | 117.082 | 1.00 | 26.90 | O |
| ATOM | 3866 | C | GLU | B | 225 | 5.839 | −20.947 | 115.052 | 1.00 | 22.83 | C |
| ATOM | 3867 | O | GLU | B | 225 | 6.101 | −20.047 | 114.268 | 1.00 | 23.16 | O |
| ATOM | 3874 | N | LEU | B | 226 | 6.420 | −22.145 | 114.998 | 1.00 | 22.42 | N |
| ATOM | 3875 | CA | LEU | B | 226 | 7.460 | −22.458 | 114.019 | 1.00 | 21.93 | C |
| ATOM | 3876 | CB | LEU | B | 226 | 7.798 | −23.954 | 114.086 | 1.00 | 21.87 | C |
| ATOM | 3877 | CG | LEU | B | 226 | 9.212 | −24.480 | 113.849 | 1.00 | 22.15 | C |
| ATOM | 3878 | CD1 | LEU | B | 226 | 9.244 | −25.574 | 112.811 | 1.00 | 21.70 | C |
| ATOM | 3879 | CD2 | LEU | B | 226 | 10.207 | −23.378 | 113.493 | 1.00 | 24.88 | C |
| ATOM | 3880 | C | LEU | B | 226 | 6.986 | −22.101 | 112.601 | 1.00 | 21.69 | C |
| ATOM | 3881 | O | LEU | B | 226 | 7.636 | −21.370 | 111.857 | 1.00 | 21.00 | O |
| ATOM | 3893 | N | ILE | B | 227 | 5.815 | −22.620 | 112.262 | 1.00 | 21.20 | N |
| ATOM | 3894 | CA | ILE | B | 227 | 5.225 | −22.383 | 110.973 | 1.00 | 20.78 | C |
| ATOM | 3895 | CB | ILE | B | 227 | 3.966 | −23.245 | 110.829 | 1.00 | 20.21 | C |
| ATOM | 3896 | CG1 | ILE | B | 227 | 4.396 | −24.708 | 110.714 | 1.00 | 20.55 | C |
| ATOM | 3897 | CD1 | ILE | B | 227 | 3.260 | −25.739 | 110.608 | 1.00 | 20.83 | C |
| ATOM | 3898 | CG2 | ILE | B | 227 | 3.174 | −22.815 | 109.627 | 1.00 | 19.79 | C |
| ATOM | 3899 | C | ILE | B | 227 | 4.951 | −20.890 | 110.734 | 1.00 | 20.95 | C |
| ATOM | 3900 | O | ILE | B | 227 | 5.220 | −20.381 | 109.645 | 1.00 | 20.68 | O |
| ATOM | 3912 | N | LEU | B | 228 | 4.410 | −20.201 | 111.734 | 1.00 | 20.97 | N |
| ATOM | 3913 | CA | LEU | B | 228 | 4.124 | −18.769 | 111.621 | 1.00 | 21.18 | C |
| ATOM | 3914 | CB | LEU | B | 228 | 3.437 | −18.247 | 112.891 | 1.00 | 21.32 | C |
| ATOM | 3915 | CG | LEU | B | 228 | 2.015 | −18.760 | 113.123 | 1.00 | 21.81 | C |
| ATOM | 3916 | CD1 | LEU | B | 228 | 1.618 | −18.507 | 114.542 | 1.00 | 22.11 | C |
| ATOM | 3917 | CD2 | LEU | B | 228 | 1.029 | −18.114 | 112.175 | 1.00 | 22.53 | C |
| ATOM | 3918 | C | LEU | B | 228 | 5.406 | −17.975 | 111.365 | 1.00 | 21.08 | C |
| ATOM | 3919 | O | LEU | B | 228 | 5.407 | −17.011 | 110.609 | 1.00 | 20.95 | O |
| ATOM | 3931 | N | GLN | B | 229 | 6.486 | −18.408 | 112.007 | 1.00 | 21.01 | N |
| ATOM | 3932 | CA | GLN | B | 229 | 7.803 | −17.833 | 111.821 | 1.00 | 20.47 | C |
| ATOM | 3933 | CB | GLN | B | 229 | 8.787 | −18.389 | 112.857 | 1.00 | 20.71 | C |
| ATOM | 3934 | CG | GLN | B | 229 | 8.431 | −18.160 | 114.332 | 1.00 | 20.12 | C |
| ATOM | 3935 | CD | GLN | B | 229 | 9.484 | −18.711 | 115.268 | 1.00 | 19.11 | C |
| ATOM | 3936 | OE1 | GLN | B | 229 | 9.476 | −20.017 | 115.482 | 1.00 | 20.97 | O |
| ATOM | 3937 | NE2 | GLN | B | 229 | 10.303 | −17.962 | 115.782 | 1.00 | 19.52 | N |
| ATOM | 3938 | C | GLN | B | 229 | 8.335 | −18.126 | 110.429 | 1.00 | 20.33 | C |
| ATOM | 3939 | O | GLN | B | 229 | 8.962 | −17.266 | 109.815 | 1.00 | 20.59 | O |
| ATOM | 3948 | N | LEU | B | 230 | 8.105 | −19.336 | 109.930 | 1.00 | 19.99 | N |
| ATOM | 3949 | CA | LEU | B | 230 | 8.521 | −19.696 | 108.577 | 1.00 | 20.01 | C |
| ATOM | 3950 | CB | LEU | B | 230 | 8.398 | −21.207 | 108.357 | 1.00 | 20.29 | C |
| ATOM | 3951 | CG | LEU | B | 230 | 9.404 | −22.015 | 109.176 | 1.00 | 21.64 | C |
| ATOM | 3952 | CD1 | LEU | B | 230 | 9.083 | −23.542 | 109.233 | 1.00 | 21.25 | C |
| ATOM | 3953 | CD2 | LEU | B | 230 | 10.800 | −21.764 | 108.624 | 1.00 | 22.88 | C |
| ATOM | 3954 | C | LEU | B | 230 | 7.757 | −18.932 | 107.498 | 1.00 | 19.57 | C |
| ATOM | 3955 | O | LEU | B | 230 | 8.309 | −18.614 | 106.448 | 1.00 | 19.66 | O |
| ATOM | 3967 | N | LEU | B | 231 | 6.498 | −18.626 | 107.755 | 1.00 | 19.07 | N |
| ATOM | 3968 | CA | LEU | B | 231 | 5.738 | −17.786 | 106.853 | 1.00 | 19.31 | C |
| ATOM | 3969 | CB | LEU | B | 231 | 4.279 | −17.775 | 107.266 | 1.00 | 19.21 | C |
| ATOM | 3970 | CG | LEU | B | 231 | 3.478 | −19.056 | 107.003 | 1.00 | 20.38 | C |
| ATOM | 3971 | CD1 | LEU | B | 231 | 2.139 | −18.987 | 107.764 | 1.00 | 21.29 | C |
| ATOM | 3972 | CD2 | LEU | B | 231 | 3.225 | −19.243 | 105.528 | 1.00 | 18.94 | C |
| ATOM | 3973 | C | LEU | B | 231 | 6.278 | −16.339 | 106.822 | 1.00 | 19.20 | C |
| ATOM | 3974 | O | LEU | B | 231 | 6.230 | −15.680 | 105.799 | 1.00 | 19.30 | O |
| ATOM | 3986 | N | GLN | B | 232 | 6.785 | −15.855 | 107.948 | 1.00 | 19.16 | N |
| ATOM | 3987 | CA | GLN | B | 232 | 7.335 | −14.496 | 108.050 | 1.00 | 19.26 | C |
| ATOM | 3988 | CB | GLN | B | 232 | 7.579 | −14.127 | 109.521 | 1.00 | 19.24 | C |
| ATOM | 3989 | CG | GLN | B | 232 | 6.354 | −13.818 | 110.373 | 1.00 | 18.42 | C |
| ATOM | 3990 | CD | GLN | B | 232 | 6.735 | −13.686 | 111.835 | 1.00 | 18.98 | C |
| ATOM | 3991 | OE1 | GLN | B | 232 | 7.601 | −12.888 | 112.169 | 1.00 | 17.49 | O |
| ATOM | 3992 | NE2 | GLN | B | 232 | 6.106 | −14.476 | 112.704 | 1.00 | 18.53 | N |
| ATOM | 3993 | C | GLN | B | 232 | 8.656 | −14.364 | 107.295 | 1.00 | 19.31 | C |
| ATOM | 3994 | O | GLN | B | 232 | 9.044 | −13.278 | 106.864 | 1.00 | 19.08 | O |
| ATOM | 4003 | N | LEU | B | 233 | 9.337 | −15.490 | 107.157 | 1.00 | 20.16 | N |
| ATOM | 4004 | CA | LEU | B | 233 | 10.654 | −15.558 | 106.552 | 1.00 | 20.76 | C |
| ATOM | 4005 | CB | LEU | B | 233 | 11.523 | −16.497 | 107.387 | 1.00 | 20.78 | C |
| ATOM | 4006 | CG | LEU | B | 233 | 11.889 | −16.001 | 108.787 | 1.00 | 20.72 | C |
| ATOM | 4007 | CD1 | LEU | B | 233 | 12.789 | −16.996 | 109.505 | 1.00 | 20.02 | C |
| ATOM | 4008 | CD2 | LEU | B | 233 | 12.563 | −14.644 | 108.720 | 1.00 | 21.85 | C |
| ATOM | 4009 | C | LEU | B | 233 | 10.637 | −16.007 | 105.076 | 1.00 | 21.49 | C |
| ATOM | 4010 | O | LEU | B | 233 | 11.672 | −15.937 | 104.389 | 1.00 | 21.23 | O |
| ATOM | 4022 | N | GLU | B | 234 | 9.463 | −16.419 | 104.587 | 1.00 | 22.11 | N |
| ATOM | 4023 | CA | GLU | B | 234 | 9.336 | −16.900 | 103.222 | 1.00 | 22.65 | C |
| ATOM | 4024 | CB | GLU | B | 234 | 7.984 | −17.599 | 103.016 | 1.00 | 22.60 | C |
| ATOM | 4025 | CG | GLU | B | 234 | 7.835 | −18.457 | 101.748 | 1.00 | 22.92 | C |
| ATOM | 4026 | CD | GLU | B | 234 | 9.022 | −19.341 | 101.425 | 1.00 | 24.14 | C |
| ATOM | 4027 | OE1 | GLU | B | 234 | 9.699 | −19.814 | 102.364 | 1.00 | 24.85 | O |
| ATOM | 4028 | OE2 | GLU | B | 234 | 9.273 | −19.589 | 100.211 | 1.00 | 26.66 | O |
| ATOM | 4029 | C | GLU | B | 234 | 9.512 | −15.742 | 102.244 | 1.00 | 23.50 | C |
| ATOM | 4030 | O | GLU | B | 234 | 8.795 | −14.729 | 102.320 | 1.00 | 23.59 | O |
| ATOM | 4037 | N | PRO | B | 235 | 10.487 | −15.865 | 101.343 | 1.00 | 24.14 | N |
| ATOM | 4038 | CA | PRO | B | 235 | 10.666 | −14.850 | 100.295 | 1.00 | 24.80 | C |
| ATOM | 4039 | CB | PRO | B | 235 | 11.736 | −15.467 | 99.369 | 1.00 | 24.74 | C |
| ATOM | 4040 | CG | PRO | B | 235 | 12.490 | −16.429 | 100.239 | 1.00 | 24.98 | C |
| ATOM | 4041 | CD | PRO | B | 235 | 11.503 | −16.934 | 101.260 | 1.00 | 24.00 | C |
| ATOM | 4042 | C | PRO | B | 235 | 9.357 | −14.603 | 99.541 | 1.00 | 24.93 | C |
| ATOM | 4043 | O | PRO | B | 235 | 8.651 | −15.555 | 99.258 | 1.00 | 24.27 | O |
| ATOM | 4051 | N | ASP | B | 236 | 9.038 | −13.347 | 99.266 | 1.00 | 25.53 | N |
| ATOM | 4052 | CA | ASP | B | 236 | 7.915 | −13.023 | 98.403 | 1.00 | 26.48 | C |
| ATOM | 4053 | CB | ASP | B | 236 | 7.587 | −11.536 | 98.496 | 1.00 | 26.83 | C |
| ATOM | 4054 | CG | ASP | B | 236 | 6.460 | −11.140 | 97.574 | 1.00 | 29.05 | C |
| ATOM | 4055 | OD1 | ASP | B | 236 | 6.504 | −10.027 | 97.012 | 1.00 | 32.21 | O |
| ATOM | 4056 | OD2 | ASP | B | 236 | 5.480 | −11.884 | 97.351 | 1.00 | 33.83 | O |
| ATOM | 4057 | C | ASP | B | 236 | 8.223 | −13.389 | 96.941 | 1.00 | 26.70 | C |
| ATOM | 4058 | O | ASP | B | 236 | 9.216 | −12.921 | 96.377 | 1.00 | 25.63 | O |
| ATOM | 4063 | N | GLU | B | 237 | 7.331 | −14.190 | 96.344 | 1.00 | 27.18 | N |
| ATOM | 4064 | CA | GLU | B | 237 | 7.522 | −14.770 | 95.001 | 1.00 | 28.02 | C |
| ATOM | 4065 | CB | GLU | B | 237 | 6.417 | −15.826 | 94.688 | 1.00 | 28.48 | C |
| ATOM | 4066 | CG | GLU | B | 237 | 6.364 | −16.373 | 93.248 | 1.00 | 31.64 | C |
| ATOM | 4067 | CD | GLU | B | 237 | 7.329 | −17.546 | 92.915 | 1.00 | 34.68 | C |
| ATOM | 4068 | OE1 | GLU | B | 237 | 8.134 | −17.985 | 93.784 | 1.00 | 36.95 | O |
| ATOM | 4069 | OE2 | GLU | B | 237 | 7.268 | −18.042 | 91.745 | 1.00 | 35.77 | O |
| ATOM | 4070 | C | GLU | B | 237 | 7.646 | −13.676 | 93.930 | 1.00 | 27.06 | C |
| ATOM | 4071 | O | GLU | B | 237 | 8.453 | −13.794 | 93.015 | 1.00 | 26.28 | O |
| ATOM | 4078 | N | ASP | B | 238 | 6.879 | −12.601 | 94.092 | 1.00 | 26.82 | N |
| ATOM | 4079 | CA | ASP | B | 238 | 6.953 | −11.443 | 93.208 | 1.00 | 26.64 | C |
| ATOM | 4080 | CB | ASP | B | 238 | 5.791 | −10.495 | 93.465 | 1.00 | 26.93 | C |
| ATOM | 4081 | CG | ASP | B | 238 | 4.453 | −11.091 | 93.063 | 1.00 | 28.54 | C |
| ATOM | 4082 | OD1 | ASP | B | 238 | 4.407 | −12.022 | 92.225 | 1.00 | 29.27 | O |
| ATOM | 4083 | OD2 | ASP | B | 238 | 3.379 | −10.688 | 93.545 | 1.00 | 33.04 | O |
| ATOM | 4084 | C | ASP | B | 238 | 8.244 | −10.664 | 93.343 | 1.00 | 26.18 | C |
| ATOM | 4085 | O | ASP | B | 238 | 8.759 | −10.154 | 92.352 | 1.00 | 26.26 | O |
| ATOM | 4090 | N | GLN | B | 239 | 8.757 | −10.556 | 94.560 | 1.00 | 25.72 | N |
| ATOM | 4091 | CA | GLN | B | 239 | 10.064 | −9.938 | 94.782 | 1.00 | 25.93 | C |
| ATOM | 4092 | CB | GLN | B | 239 | 10.295 | −9.653 | 96.268 | 1.00 | 25.95 | C |
| ATOM | 4093 | CG | GLN | B | 239 | 9.386 | −8.587 | 96.835 | 1.00 | 25.99 | C |
| ATOM | 4094 | CD | GLN | B | 239 | 9.748 | −8.254 | 98.248 | 1.00 | 26.98 | C |
| ATOM | 4095 | OE1 | GLN | B | 239 | 10.007 | −9.152 | 99.052 | 1.00 | 27.22 | O |
| ATOM | 4096 | NE2 | GLN | B | 239 | 9.817 | −6.965 | 98.554 | 1.00 | 27.89 | N |
| ATOM | 4097 | C | GLN | B | 239 | 11.224 | −10.784 | 94.270 | 1.00 | 26.07 | C |
| ATOM | 4098 | O | GLN | B | 239 | 12.159 | −10.256 | 93.663 | 1.00 | 26.46 | O |
| ATOM | 4107 | N | VAL | B | 240 | 11.191 | −12.082 | 94.538 | 1.00 | 26.27 | N |
| ATOM | 4108 | CA | VAL | B | 240 | 12.240 | −12.951 | 94.028 | 1.00 | 26.69 | C |
| ATOM | 4109 | CB | VAL | B | 240 | 12.248 | −14.387 | 94.673 | 1.00 | 26.92 | C |
| ATOM | 4110 | CG1 | VAL | B | 240 | 10.897 | −14.839 | 95.030 | 1.00 | 29.16 | C |
| ATOM | 4111 | CG2 | VAL | B | 240 | 12.905 | −15.439 | 93.750 | 1.00 | 26.94 | C |
| ATOM | 4112 | C | VAL | B | 240 | 12.227 | −12.933 | 92.486 | 1.00 | 26.20 | C |
| ATOM | 4113 | O | VAL | B | 240 | 13.283 | −12.999 | 91.871 | 1.00 | 26.29 | O |
| ATOM | 4123 | N | ARG | B | 241 | 11.057 | −12.770 | 91.874 | 1.00 | 25.75 | N |
| ATOM | 4124 | CA | ARG | B | 241 | 10.959 | −12.734 | 90.412 | 1.00 | 25.67 | C |
| ATOM | 4125 | CB | ARG | B | 241 | 9.494 | −12.731 | 89.960 | 1.00 | 25.76 | C |
| ATOM | 4126 | CG | ARG | B | 241 | 9.271 | −12.724 | 88.449 | 1.00 | 26.11 | C |
| ATOM | 4127 | CD | ARG | B | 241 | 7.814 | −12.465 | 87.997 | 1.00 | 27.93 | C |
| ATOM | 4128 | NE | ARG | B | 241 | 6.821 | −12.689 | 89.049 | 1.00 | 30.76 | N |
| ATOM | 4129 | CZ | ARG | B | 241 | 6.506 | −13.897 | 89.556 | 1.00 | 34.28 | C |
| ATOM | 4130 | NH1 | ARG | B | 241 | 7.097 | −15.017 | 89.113 | 1.00 | 36.71 | N |
| ATOM | 4131 | NH2 | ARG | B | 241 | 5.602 | −14.001 | 90.527 | 1.00 | 34.68 | N |
| ATOM | 4132 | C | ARG | B | 241 | 11.679 | −11.518 | 89.866 | 1.00 | 25.51 | C |
| ATOM | 4133 | O | ARG | B | 241 | 12.516 | −11.639 | 88.977 | 1.00 | 25.81 | O |
| ATOM | 4147 | N | ALA | B | 242 | 11.364 | −10.346 | 90.415 | 1.00 | 25.33 | N |
| ATOM | 4148 | CA | ALA | B | 242 | 11.949 | −9.083 | 89.965 | 1.00 | 25.05 | C |
| ATOM | 4149 | CB | ALA | B | 242 | 11.292 | −7.913 | 90.689 | 1.00 | 24.75 | C |
| ATOM | 4150 | C | ALA | B | 242 | 13.465 | −9.051 | 90.168 | 1.00 | 25.00 | C |
| ATOM | 4151 | O | ALA | B | 242 | 14.211 | −8.578 | 89.315 | 1.00 | 25.04 | O |
| ATOM | 4157 | N | ARG | B | 243 | 13.901 | −9.552 | 91.311 | 1.00 | 24.95 | N |
| ATOM | 4158 | CA | ARG | B | 243 | 15.311 | −9.680 | 91.628 | 1.00 | 25.22 | C |
| ATOM | 4159 | CB | ARG | B | 243 | 15.409 | −10.298 | 93.016 | 1.00 | 25.32 | C |
| ATOM | 4160 | CG | ARG | B | 243 | 16.733 | −10.147 | 93.696 | 1.00 | 26.31 | C |
| ATOM | 4161 | CD | ARG | B | 243 | 16.621 | −10.291 | 95.220 | 1.00 | 28.18 | C |
| ATOM | 4162 | NE | ARG | B | 243 | 16.664 | −8.989 | 95.904 | 1.00 | 29.87 | N |
| ATOM | 4163 | CZ | ARG | B | 243 | 16.338 | −8.776 | 97.186 | 1.00 | 30.13 | C |
| ATOM | 4164 | NH1 | ARG | B | 243 | 15.930 | −9.779 | 97.973 | 1.00 | 30.42 | N |
| ATOM | 4165 | NH2 | ARG | B | 243 | 16.417 | −7.539 | 97.680 | 1.00 | 30.30 | N |
| ATOM | 4166 | C | ARG | B | 243 | 16.065 | −10.541 | 90.581 | 1.00 | 25.49 | C |
| ATOM | 4167 | O | ARG | B | 243 | 17.077 | −10.113 | 90.011 | 1.00 | 25.81 | O |
| ATOM | 4181 | N | ILE | B | 244 | 15.536 | −11.736 | 90.307 | 1.00 | 25.78 | N |
| ATOM | 4182 | CA | ILE | B | 244 | 16.163 | −12.696 | 89.380 | 1.00 | 25.53 | C |
| ATOM | 4183 | CB | ILE | B | 244 | 15.458 | −14.065 | 89.489 | 1.00 | 25.26 | C |
| ATOM | 4184 | CG1 | ILE | B | 244 | 15.813 | −14.692 | 90.831 | 1.00 | 25.22 | C |
| ATOM | 4185 | CD1 | ILE | B | 244 | 15.135 | −16.015 | 91.141 | 1.00 | 25.46 | C |
| ATOM | 4186 | CG2 | ILE | B | 244 | 15.849 | −14.989 | 88.339 | 1.00 | 24.80 | C |
| ATOM | 4187 | C | ILE | B | 244 | 16.153 | −12.176 | 87.930 | 1.00 | 25.79 | C |
| ATOM | 4188 | O | ILE | B | 244 | 17.169 | −12.243 | 87.226 | 1.00 | 25.26 | O |
| ATOM | 4200 | N | LEU | B | 245 | 14.987 | −11.677 | 87.512 | 1.00 | 25.76 | N |
| ATOM | 4201 | CA | LEU | B | 245 | 14.799 | −11.006 | 86.235 | 1.00 | 25.85 | C |
| ATOM | 4202 | CB | LEU | B | 245 | 13.393 | −10.405 | 86.181 | 1.00 | 26.41 | C |
| ATOM | 4203 | CG | LEU | B | 245 | 12.350 | −10.979 | 85.208 | 1.00 | 26.92 | C |
| ATOM | 4204 | CD1 | LEU | B | 245 | 12.548 | −12.451 | 84.952 | 1.00 | 27.67 | C |
| ATOM | 4205 | CD2 | LEU | B | 245 | 10.912 | −10.684 | 85.705 | 1.00 | 26.22 | C |
| ATOM | 4206 | C | LEU | B | 245 | 15.820 | −9.895 | 86.023 | 1.00 | 25.80 | C |
| ATOM | 4207 | O | LEU | B | 245 | 16.411 | −9.767 | 84.947 | 1.00 | 25.15 | O |
| ATOM | 4219 | N | GLY | B | 246 | 16.028 | −9.107 | 87.074 | 1.00 | 25.97 | N |
| ATOM | 4220 | CA | GLY | B | 246 | 17.008 | −8.035 | 87.072 | 1.00 | 25.92 | C |
| ATOM | 4221 | C | GLY | B | 246 | 18.441 | −8.534 | 87.052 | 1.00 | 25.78 | C |
| ATOM | 4222 | O | GLY | B | 246 | 19.299 | −7.921 | 86.428 | 1.00 | 25.80 | O |
| ATOM | 4226 | N | SER | B | 247 | 18.705 | −9.650 | 87.724 | 1.00 | 25.86 | N |
| ATOM | 4227 | CA | SER | B | 247 | 20.010 | −10.282 | 87.608 | 1.00 | 26.04 | C |
| ATOM | 4228 | CB | SER | B | 247 | 20.172 | −11.388 | 88.624 | 1.00 | 26.06 | C |
| ATOM | 4229 | OG | SER | B | 247 | 21.495 | −11.882 | 88.547 | 1.00 | 26.68 | O |
| ATOM | 4230 | C | SER | B | 247 | 20.270 | −10.850 | 86.207 | 1.00 | 26.14 | C |
| ATOM | 4231 | O | SER | B | 247 | 21.403 | −10.828 | 85.718 | 1.00 | 26.35 | O |
| ATOM | 4237 | N | LEU | B | 248 | 19.225 | −11.327 | 85.544 | 1.00 | 26.41 | N |
| ATOM | 4238 | CA | LEU | B | 248 | 19.384 | −11.906 | 84.211 | 1.00 | 26.78 | C |
| ATOM | 4239 | CB | LEU | B | 248 | 18.210 | −12.838 | 83.870 | 1.00 | 26.62 | C |
| ATOM | 4240 | CG | LEU | B | 248 | 18.071 | −14.089 | 84.758 | 1.00 | 25.74 | C |
| ATOM | 4241 | CD1 | LEU | B | 248 | 16.844 | −14.895 | 84.349 | 1.00 | 25.23 | C |
| ATOM | 4242 | CD2 | LEU | B | 248 | 19.350 | −14.938 | 84.729 | 1.00 | 24.89 | C |
| ATOM | 4243 | C | LEU | B | 248 | 19.553 | −10.838 | 83.140 | 1.00 | 27.21 | C |
| ATOM | 4244 | O | LEU | B | 248 | 19.745 | −11.169 | 81.974 | 1.00 | 27.26 | O |
| ATOM | 4256 | N | GLN | B | 249 | 19.497 | −9.569 | 83.546 | 1.00 | 28.10 | N |
| ATOM | 4257 | CA | GLN | B | 249 | 19.713 | −8.433 | 82.645 | 1.00 | 28.88 | C |
| ATOM | 4258 | CB | GLN | B | 249 | 18.745 | −7.288 | 82.998 | 1.00 | 29.21 | C |
| ATOM | 4259 | CG | GLN | B | 249 | 17.311 | −7.503 | 82.458 | 1.00 | 30.45 | C |
| ATOM | 4260 | CD | GLN | B | 249 | 17.294 | −7.928 | 80.981 | 1.00 | 31.79 | C |
| ATOM | 4261 | OE1 | GLN | B | 249 | 17.483 | −7.094 | 80.085 | 1.00 | 34.35 | O |
| ATOM | 4262 | NE2 | GLN | B | 249 | 17.092 | −9.223 | 80.732 | 1.00 | 31.33 | N |
| ATOM | 4263 | C | GLN | B | 249 | 21.158 | −7.906 | 82.597 | 1.00 | 29.18 | C |
| ATOM | 4264 | O | GLN | B | 249 | 21.516 | −7.172 | 81.674 | 1.00 | 28.86 | O |
| ATOM | 4273 | N | GLU | B | 250 | 21.976 | −8.277 | 83.581 | 1.00 | 29.76 | N |
| ATOM | 4274 | CA | GLU | B | 250 | 23.415 | −7.966 | 83.564 | 1.00 | 30.10 | C |
| ATOM | 4275 | CB | GLU | B | 250 | 24.058 | −8.418 | 84.888 | 1.00 | 30.29 | C |
| ATOM | 4276 | CG | GLU | B | 250 | 23.585 | −7.630 | 86.105 | 1.00 | 30.79 | C |
| ATOM | 4277 | CD | GLU | B | 250 | 23.689 | −8.393 | 87.420 | 1.00 | 33.08 | C |
| ATOM | 4278 | OE1 | GLU | B | 250 | 23.865 | −9.636 | 87.435 | 1.00 | 34.21 | O |
| ATOM | 4279 | OE2 | GLU | B | 250 | 23.573 | −7.733 | 88.469 | 1.00 | 35.79 | O |
| ATOM | 4280 | C | GLU | B | 250 | 24.107 | −8.664 | 82.369 | 1.00 | 30.16 | C |
| ATOM | 4281 | O | GLU | B | 250 | 23.740 | −9.796 | 82.034 | 1.00 | 30.25 | O |
| ATOM | 4288 | N | PRO | B | 251 | 25.090 | −8.022 | 81.726 | 1.00 | 30.59 | N |
| ATOM | 4289 | CA | PRO | B | 251 | 25.752 | −8.638 | 80.561 | 1.00 | 30.87 | C |
| ATOM | 4290 | CB | PRO | B | 251 | 26.604 | −7.500 | 79.992 | 1.00 | 30.78 | C |
| ATOM | 4291 | CG | PRO | B | 251 | 26.175 | −6.258 | 80.726 | 1.00 | 30.53 | C |
| ATOM | 4292 | CD | PRO | B | 251 | 25.664 | −6.698 | 82.040 | 1.00 | 30.32 | C |
| ATOM | 4293 | C | PRO | B | 251 | 26.639 | −9.841 | 80.940 | 1.00 | 31.51 | C |
| ATOM | 4294 | O | PRO | B | 251 | 27.405 | −9.750 | 81.911 | 1.00 | 32.17 | O |
| ATOM | 4302 | N | THR | B | 252 | 26.512 | −10.954 | 80.214 | 1.00 | 31.67 | N |
| ATOM | 4303 | CA | THR | B | 252 | 27.392 | −12.108 | 80.411 | 1.00 | 31.74 | C |
| ATOM | 4304 | CB | THR | B | 252 | 26.605 | −13.427 | 80.286 | 1.00 | 31.77 | C |
| ATOM | 4305 | OG1 | THR | B | 252 | 25.979 | −13.713 | 81.541 | 1.00 | 32.51 | O |
| ATOM | 4306 | CG2 | THR | B | 252 | 27.533 | −14.653 | 80.043 | 1.00 | 31.27 | C |
| ATOM | 4307 | C | THR | B | 252 | 28.511 | −12.020 | 79.384 | 1.00 | 32.04 | C |
| ATOM | 4308 | O | THR | B | 252 | 29.630 | −11.559 | 79.698 | 1.00 | 32.32 | O |
| ATOM | 4316 | N | LYS | B | 253 | 28.197 | −12.437 | 78.151 | 1.00 | 32.10 | N |
| ATOM | 4317 | CA | LYS | B | 253 | 29.122 | −12.315 | 77.019 | 1.00 | 31.72 | C |
| ATOM | 4318 | CB | LYS | B | 253 | 30.291 | −13.309 | 77.185 | 1.00 | 31.78 | C |
| ATOM | 4319 | CG | LYS | B | 253 | 31.667 | −12.749 | 76.816 | 1.00 | 30.29 | C |
| ATOM | 4320 | CD | LYS | B | 253 | 32.462 | −13.703 | 75.916 | 1.00 | 29.61 | C |
| ATOM | 4321 | CE | LYS | B | 253 | 33.966 | −13.500 | 76.093 | 1.00 | 29.68 | C |
| ATOM | 4322 | NZ | LYS | B | 253 | 34.733 | −13.530 | 74.815 | 1.00 | 30.02 | N |
| ATOM | 4323 | C | LYS | B | 253 | 28.448 | −12.560 | 75.657 | 1.00 | 31.80 | C |
| ATOM | 4324 | O | LYS | B | 253 | 27.229 | −12.819 | 75.556 | 1.00 | 31.64 | O |
| ATOM | 4338 | N | SER | B | 254 | 29.270 | −12.459 | 74.614 | 1.00 | 31.73 | N |
| ATOM | 4339 | CA | SER | B | 254 | 28.935 | −12.967 | 73.289 | 1.00 | 31.86 | C |
| ATOM | 4340 | CB | SER | B | 254 | 29.317 | −11.949 | 72.204 | 1.00 | 31.96 | C |
| ATOM | 4341 | OG | SER | B | 254 | 29.110 | −12.473 | 70.896 | 1.00 | 33.14 | O |
| ATOM | 4342 | C | SER | B | 254 | 29.671 | −14.288 | 73.078 | 1.00 | 31.76 | C |
| ATOM | 4343 | O | SER | B | 254 | 30.772 | −14.309 | 72.524 | 1.00 | 31.94 | O |
| ATOM | 4349 | N | ARG | B | 255 | 29.085 | −15.380 | 73.569 | 1.00 | 31.90 | N |
| ATOM | 4350 | CA | ARG | B | 255 | 29.529 | −16.722 | 73.188 | 1.00 | 31.79 | C |
| ATOM | 4351 | CB | ARG | B | 255 | 29.758 | −17.617 | 74.422 | 1.00 | 31.63 | C |
| ATOM | 4352 | CG | ARG | B | 255 | 31.254 | −17.913 | 74.687 | 1.00 | 31.94 | C |
| ATOM | 4353 | CD | ARG | B | 255 | 31.500 | −19.125 | 75.587 | 1.00 | 32.68 | C |
| ATOM | 4354 | NE | ARG | B | 255 | 32.458 | −18.879 | 76.680 | 1.00 | 33.33 | N |
| ATOM | 4355 | CZ | ARG | B | 255 | 32.238 | −19.120 | 77.997 | 1.00 | 32.79 | C |
| ATOM | 4356 | NH1 | ARG | B | 255 | 31.074 | −19.622 | 78.454 | 1.00 | 32.41 | N |
| ATOM | 4357 | NH2 | ARG | B | 255 | 33.210 | −18.852 | 78.875 | 1.00 | 32.93 | N |
| ATOM | 4358 | C | ARG | B | 255 | 28.484 | −17.321 | 72.234 | 1.00 | 31.84 | C |
| ATOM | 4359 | O | ARG | B | 255 | 27.565 | −18.014 | 72.704 | 1.00 | 31.90 | O |
| ATOM | 4373 | N | PRO | B | 256 | 28.622 | −17.045 | 70.914 | 1.00 | 31.79 | N |
| ATOM | 4374 | CA | PRO | B | 256 | 27.676 | −17.532 | 69.882 | 1.00 | 31.85 | C |
| ATOM | 4375 | CB | PRO | B | 256 | 27.965 | −16.599 | 68.683 | 1.00 | 31.56 | C |
| ATOM | 4376 | CG | PRO | B | 256 | 29.434 | −16.268 | 68.798 | 1.00 | 31.38 | C |
| ATOM | 4377 | CD | PRO | B | 256 | 29.704 | −16.240 | 70.295 | 1.00 | 31.59 | C |
| ATOM | 4378 | C | PRO | B | 256 | 27.858 | −19.026 | 69.485 | 1.00 | 32.20 | C |
| ATOM | 4379 | O | PRO | B | 256 | 27.878 | −19.350 | 68.288 | 1.00 | 32.00 | O |
| ATOM | 4387 | N | ASP | B | 257 | 27.963 | −19.903 | 70.495 | 1.00 | 32.63 | N |
| ATOM | 4388 | CA | ASP | B | 257 | 28.268 | −21.333 | 70.316 | 1.00 | 33.05 | C |
| ATOM | 4389 | CB | ASP | B | 257 | 29.732 | −21.709 | 70.691 | 1.00 | 33.60 | C |
| ATOM | 4390 | CG | ASP | B | 257 | 30.508 | −20.591 | 71.444 | 1.00 | 35.52 | C |
| ATOM | 4391 | OD1 | ASP | B | 257 | 30.423 | −19.395 | 71.047 | 1.00 | 37.38 | O |
| ATOM | 4392 | OD2 | ASP | B | 257 | 31.276 | −20.834 | 72.431 | 1.00 | 38.32 | O |
| ATOM | 4393 | C | ASP | B | 257 | 27.311 | −22.161 | 71.157 | 1.00 | 32.77 | C |
| ATOM | 4394 | O | ASP | B | 257 | 26.663 | −23.093 | 70.667 | 1.00 | 33.06 | O |
| ATOM | 4399 | N | GLN | B | 258 | 27.252 | −21.828 | 72.441 | 1.00 | 32.31 | N |
| ATOM | 4400 | CA | GLN | B | 258 | 26.251 | −22.396 | 73.325 | 1.00 | 31.68 | C |
| ATOM | 4401 | CB | GLN | B | 258 | 26.753 | −22.450 | 74.776 | 1.00 | 31.98 | C |
| ATOM | 4402 | CG | GLN | B | 258 | 27.931 | −23.456 | 75.022 | 1.00 | 32.49 | C |
| ATOM | 4403 | CD | GLN | B | 258 | 27.462 | −24.917 | 75.227 | 1.00 | 33.03 | C |
| ATOM | 4404 | OE1 | GLN | B | 258 | 26.432 | −25.175 | 75.893 | 1.00 | 32.90 | O |
| ATOM | 4405 | NE2 | GLN | B | 258 | 28.217 | −25.867 | 74.653 | 1.00 | 32.76 | N |
| ATOM | 4406 | C | GLN | B | 258 | 24.943 | −21.592 | 73.235 | 1.00 | 30.93 | C |
| ATOM | 4407 | O | GLN | B | 258 | 24.958 | −20.360 | 73.019 | 1.00 | 30.52 | O |
| ATOM | 4416 | N | PRO | B | 259 | 23.818 | −22.295 | 73.412 | 1.00 | 29.83 | N |
| ATOM | 4417 | CA | PRO | B | 259 | 22.490 | −21.666 | 73.338 | 1.00 | 28.91 | C |
| ATOM | 4418 | CB | PRO | B | 259 | 21.508 | −22.821 | 73.571 | 1.00 | 29.03 | C |
| ATOM | 4419 | CG | PRO | B | 259 | 22.303 | −24.074 | 73.427 | 1.00 | 29.43 | C |
| ATOM | 4420 | CD | PRO | B | 259 | 23.735 | −23.735 | 73.720 | 1.00 | 29.67 | C |
| ATOM | 4421 | C | PRO | B | 259 | 22.354 | −20.651 | 74.435 | 1.00 | 28.06 | C |
| ATOM | 4422 | O | PRO | B | 259 | 22.611 | −20.970 | 75.598 | 1.00 | 28.12 | O |
| ATOM | 4430 | N | ALA | B | 260 | 21.985 | −19.438 | 74.052 | 1.00 | 27.13 | N |
| ATOM | 4431 | CA | ALA | B | 260 | 21.848 | −18.339 | 74.986 | 1.00 | 26.40 | C |
| ATOM | 4432 | CB | ALA | B | 260 | 21.372 | −17.088 | 74.252 | 1.00 | 26.54 | C |
| ATOM | 4433 | C | ALA | B | 260 | 20.899 | −18.700 | 76.116 | 1.00 | 25.81 | C |
| ATOM | 4434 | O | ALA | B | 260 | 21.194 | −18.439 | 77.278 | 1.00 | 26.30 | O |
| ATOM | 4440 | N | ALA | B | 261 | 19.784 | −19.332 | 75.778 | 1.00 | 25.02 | N |
| ATOM | 4441 | CA | ALA | B | 261 | 18.767 | −19.680 | 76.765 | 1.00 | 24.72 | C |
| ATOM | 4442 | CB | ALA | B | 261 | 17.485 | −20.139 | 76.074 | 1.00 | 24.62 | C |
| ATOM | 4443 | C | ALA | B | 261 | 19.216 | −20.738 | 77.771 | 1.00 | 24.72 | C |
| ATOM | 4444 | O | ALA | B | 261 | 18.689 | −20.802 | 78.881 | 1.00 | 24.97 | O |
| ATOM | 4450 | N | PHE | B | 262 | 20.160 | −21.593 | 77.407 | 1.00 | 24.33 | N |
| ATOM | 4451 | CA | PHE | B | 262 | 20.663 | −22.546 | 78.384 | 1.00 | 23.79 | C |
| ATOM | 4452 | CB | PHE | B | 262 | 21.550 | −23.604 | 77.739 | 1.00 | 23.95 | C |
| ATOM | 4453 | CG | PHE | B | 262 | 22.042 | −24.609 | 78.719 | 1.00 | 24.92 | C |
| ATOM | 4454 | CD1 | PHE | B | 262 | 21.216 | −25.642 | 79.132 | 1.00 | 25.79 | C |
| ATOM | 4455 | CE1 | PHE | B | 262 | 21.655 | −26.557 | 80.076 | 1.00 | 26.48 | C |
| ATOM | 4456 | CZ | PHE | B | 262 | 22.920 | −26.435 | 80.630 | 1.00 | 25.35 | C |
| ATOM | 4457 | CE2 | PHE | B | 262 | 23.744 | −25.402 | 80.237 | 1.00 | 25.60 | C |
| ATOM | 4458 | CD2 | PHE | B | 262 | 23.305 | −24.486 | 79.294 | 1.00 | 25.44 | C |
| ATOM | 4459 | C | PHE | B | 262 | 21.426 | −21.836 | 79.516 | 1.00 | 23.18 | C |
| ATOM | 4460 | O | PHE | B | 262 | 21.218 | −22.122 | 80.678 | 1.00 | 21.96 | O |
| ATOM | 4470 | N | GLY | B | 263 | 22.302 | −20.895 | 79.172 | 1.00 | 23.10 | N |
| ATOM | 4471 | CA | GLY | B | 263 | 23.004 | −20.124 | 80.192 | 1.00 | 22.98 | C |
| ATOM | 4472 | C | GLY | B | 263 | 22.056 | −19.312 | 81.068 | 1.00 | 22.85 | C |
| ATOM | 4473 | O | GLY | B | 263 | 22.218 | −19.202 | 82.284 | 1.00 | 22.43 | O |
| ATOM | 4477 | N | LEU | B | 264 | 21.057 | −18.749 | 80.408 | 1.00 | 22.77 | N |
| ATOM | 4478 | CA | LEU | B | 264 | 20.025 | −17.948 | 81.026 | 1.00 | 22.80 | C |
| ATOM | 4479 | CB | LEU | B | 264 | 19.038 | −17.532 | 79.926 | 1.00 | 23.46 | C |
| ATOM | 4480 | CG | LEU | B | 264 | 17.982 | −16.455 | 80.174 | 1.00 | 25.74 | C |
| ATOM | 4481 | CD1 | LEU | B | 264 | 16.650 | −17.072 | 80.600 | 1.00 | 27.18 | C |
| ATOM | 4482 | CD2 | LEU | B | 264 | 18.472 | −15.427 | 81.203 | 1.00 | 27.38 | C |
| ATOM | 4483 | C | LEU | B | 264 | 19.299 | −18.714 | 82.129 | 1.00 | 22.14 | C |
| ATOM | 4484 | O | LEU | B | 264 | 19.129 | −18.215 | 83.233 | 1.00 | 21.59 | O |
| ATOM | 4496 | N | LEU | B | 265 | 18.875 | −19.926 | 81.819 | 1.00 | 21.31 | N |
| ATOM | 4497 | CA | LEU | B | 265 | 18.162 | −20.756 | 82.762 | 1.00 | 21.53 | C |
| ATOM | 4498 | CB | LEU | B | 265 | 17.541 | −21.938 | 82.038 | 1.00 | 22.06 | C |
| ATOM | 4499 | CG | LEU | B | 265 | 16.464 | −21.551 | 81.030 | 1.00 | 24.04 | C |
| ATOM | 4500 | CD1 | LEU | B | 265 | 16.233 | −22.671 | 79.989 | 1.00 | 24.63 | C |
| ATOM | 4501 | CD2 | LEU | B | 265 | 15.186 | −21.197 | 81.781 | 1.00 | 25.12 | C |
| ATOM | 4502 | C | LEU | B | 265 | 19.061 | −21.296 | 83.861 | 1.00 | 20.95 | C |
| ATOM | 4503 | O | LEU | B | 265 | 18.632 | −21.505 | 84.974 | 1.00 | 20.80 | O |
| ATOM | 4515 | N | CYS | B | 266 | 20.303 | −21.568 | 83.513 | 1.00 | 21.01 | N |
| ATOM | 4516 | CA | CYS | B | 266 | 21.334 | −21.879 | 84.483 | 1.00 | 21.08 | C |
| ATOM | 4517 | CB | CYS | B | 266 | 22.656 | −22.159 | 83.770 | 1.00 | 21.01 | C |
| ATOM | 4518 | SG | CYS | B | 266 | 22.743 | −23.763 | 82.953 | 1.00 | 22.23 | S |
| ATOM | 4519 | C | CYS | B | 266 | 21.512 | −20.723 | 85.469 | 1.00 | 20.65 | C |
| ATOM | 4520 | O | CYS | B | 266 | 21.617 | −20.947 | 86.654 | 1.00 | 20.45 | O |
| ATOM | 4526 | N | ARG | B | 267 | 21.519 | −19.491 | 84.979 | 1.00 | 20.57 | N |
| ATOM | 4527 | CA | ARG | B | 267 | 21.673 | −18.336 | 85.864 | 1.00 | 20.75 | C |
| ATOM | 4528 | CB | ARG | B | 267 | 21.913 | −17.056 | 85.088 | 1.00 | 20.51 | C |
| ATOM | 4529 | CG | ARG | B | 267 | 23.341 | −16.948 | 84.666 | 1.00 | 22.77 | C |
| ATOM | 4530 | CD | ARG | B | 267 | 23.698 | −15.646 | 84.008 | 1.00 | 25.81 | C |
| ATOM | 4531 | NE | ARG | B | 267 | 23.247 | −15.635 | 82.628 | 1.00 | 29.25 | N |
| ATOM | 4532 | CZ | ARG | B | 267 | 23.061 | −14.543 | 81.906 | 1.00 | 31.89 | C |
| ATOM | 4533 | NH1 | ARG | B | 267 | 23.287 | −13.329 | 82.430 | 1.00 | 32.32 | N |
| ATOM | 4534 | NH2 | ARG | B | 267 | 22.643 | −14.671 | 80.646 | 1.00 | 33.23 | N |
| ATOM | 4535 | C | ARG | B | 267 | 20.497 | −18.165 | 86.750 | 1.00 | 20.44 | C |
| ATOM | 4536 | O | ARG | B | 267 | 20.648 | −17.828 | 87.900 | 1.00 | 20.41 | O |
| ATOM | 4550 | N | MET | B | 268 | 19.323 | −18.420 | 86.196 | 1.00 | 20.87 | N |
| ATOM | 4551 | CA | MET | B | 268 | 18.049 | −18.304 | 86.896 | 1.00 | 20.78 | C |
| ATOM | 4552 | CB | MET | B | 268 | 16.910 | −18.557 | 85.899 | 1.00 | 20.72 | C |
| ATOM | 4553 | CG | MET | B | 268 | 15.541 | −18.773 | 86.512 | 1.00 | 21.70 | C |
| ATOM | 4554 | SD | MET | B | 268 | 14.329 | −19.464 | 85.345 | 1.00 | 21.30 | S |
| ATOM | 4555 | CE | MET | B | 268 | 14.718 | −21.205 | 85.689 | 1.00 | 21.55 | C |
| ATOM | 4556 | C | MET | B | 268 | 17.952 | −19.255 | 88.091 | 1.00 | 20.46 | C |
| ATOM | 4557 | O | MET | B | 268 | 17.405 | −18.898 | 89.134 | 1.00 | 19.88 | O |
| ATOM | 4567 | N | ALA | B | 269 | 18.466 | −20.470 | 87.921 | 1.00 | 20.66 | N |
| ATOM | 4568 | CA | ALA | B | 269 | 18.458 | −21.467 | 88.971 | 1.00 | 20.74 | C |
| ATOM | 4569 | CB | ALA | B | 269 | 18.678 | −22.832 | 88.390 | 1.00 | 21.23 | C |
| ATOM | 4570 | C | ALA | B | 269 | 19.534 | −21.170 | 90.018 | 1.00 | 20.85 | C |
| ATOM | 4571 | O | ALA | B | 269 | 19.358 | −21.475 | 91.183 | 1.00 | 21.64 | O |
| ATOM | 4577 | N | ASP | B | 270 | 20.648 | −20.602 | 89.587 | 1.00 | 20.75 | N |
| ATOM | 4578 | CA | ASP | B | 270 | 21.683 | −20.128 | 90.477 | 1.00 | 21.05 | C |
| ATOM | 4579 | CB | ASP | B | 270 | 22.843 | −19.503 | 89.668 | 1.00 | 21.42 | C |
| ATOM | 4580 | CG | ASP | B | 270 | 23.945 | −20.491 | 89.328 | 1.00 | 21.19 | C |
| ATOM | 4581 | OD1 | ASP | B | 270 | 23.745 | −21.703 | 89.517 | 1.00 | 20.42 | O |
| ATOM | 4582 | OD2 | ASP | B | 270 | 25.047 | −20.130 | 88.856 | 1.00 | 21.50 | O |
| ATOM | 4583 | C | ASP | B | 270 | 21.108 | −19.059 | 91.397 | 1.00 | 20.84 | C |
| ATOM | 4584 | O | ASP | B | 270 | 21.316 | −19.076 | 92.596 | 1.00 | 20.99 | O |
| ATOM | 4589 | N | GLN | B | 271 | 20.388 | −18.121 | 90.821 | 1.00 | 20.68 | N |
| ATOM | 4590 | CA | GLN | B | 271 | 19.858 | −17.002 | 91.569 | 1.00 | 20.63 | C |
| ATOM | 4591 | CB | GLN | B | 271 | 19.355 | −15.925 | 90.608 | 1.00 | 20.67 | C |
| ATOM | 4592 | CG | GLN | B | 271 | 20.437 | −15.150 | 89.898 | 1.00 | 20.94 | C |
| ATOM | 4593 | CD | GLN | B | 271 | 21.456 | −14.543 | 90.867 | 1.00 | 21.35 | C |
| ATOM | 4594 | OE1 | GLN | B | 271 | 22.659 | −14.652 | 90.645 | 1.00 | 21.04 | O |
| ATOM | 4595 | NE2 | GLN | B | 271 | 20.972 | −13.933 | 91.946 | 1.00 | 20.11 | N |
| ATOM | 4596 | C | GLN | B | 271 | 18.739 | −17.444 | 92.488 | 1.00 | 20.52 | C |
| ATOM | 4597 | O | GLN | B | 271 | 18.476 | −16.802 | 93.505 | 1.00 | 20.27 | O |
| ATOM | 4606 | N | THR | B | 272 | 18.082 | −18.544 | 92.134 | 1.00 | 20.47 | N |
| ATOM | 4607 | CA | THR | B | 272 | 17.007 | −19.097 | 92.952 | 1.00 | 20.43 | C |
| ATOM | 4608 | CB | THR | B | 272 | 16.229 | −20.243 | 92.209 | 1.00 | 20.20 | C |
| ATOM | 4609 | OG1 | THR | B | 272 | 15.725 | −19.788 | 90.943 | 1.00 | 20.34 | O |
| ATOM | 4610 | CG2 | THR | B | 272 | 14.995 | −20.634 | 92.979 | 1.00 | 19.22 | C |
| ATOM | 4611 | C | THR | B | 272 | 17.646 | −19.667 | 94.189 | 1.00 | 20.68 | C |
| ATOM | 4612 | O | THR | B | 272 | 17.159 | −19.507 | 95.306 | 1.00 | 20.86 | O |
| ATOM | 4620 | N | PHE | B | 273 | 18.759 | −20.350 | 93.964 | 1.00 | 20.91 | N |
| ATOM | 4621 | CA | PHE | B | 273 | 19.492 | −20.963 | 95.040 | 1.00 | 20.80 | C |
| ATOM | 4622 | CB | PHE | B | 273 | 20.555 | −21.928 | 94.513 | 1.00 | 20.56 | C |
| ATOM | 4623 | CG | PHE | B | 273 | 21.254 | −22.652 | 95.608 | 1.00 | 21.32 | C |
| ATOM | 4624 | CD1 | PHE | B | 273 | 20.551 | −23.531 | 96.410 | 1.00 | 20.50 | C |
| ATOM | 4625 | CE1 | PHE | B | 273 | 21.171 | −24.156 | 97.462 | 1.00 | 21.33 | C |
| ATOM | 4626 | CZ | PHE | B | 273 | 22.500 | −23.907 | 97.724 | 1.00 | 20.08 | C |
| ATOM | 4627 | CE2 | PHE | B | 273 | 23.203 | −23.006 | 96.939 | 1.00 | 20.71 | C |
| ATOM | 4628 | CD2 | PHE | B | 273 | 22.583 | −22.385 | 95.901 | 1.00 | 19.85 | C |
| ATOM | 4629 | C | PHE | B | 273 | 20.101 | −19.915 | 95.965 | 1.00 | 20.36 | C |
| ATOM | 4630 | O | PHE | B | 273 | 20.071 | −20.083 | 97.173 | 1.00 | 20.94 | O |
| ATOM | 4640 | N | ILE | B | 274 | 20.648 | −18.838 | 95.417 | 1.00 | 19.99 | N |
| ATOM | 4641 | CA | ILE | B | 274 | 21.098 | −17.724 | 96.242 | 1.00 | 20.19 | C |
| ATOM | 4642 | CB | ILE | B | 274 | 21.673 | −16.597 | 95.348 | 1.00 | 19.88 | C |
| ATOM | 4643 | CG1 | ILE | B | 274 | 23.068 | −16.996 | 94.845 | 1.00 | 19.71 | C |
| ATOM | 4644 | CD1 | ILE | B | 274 | 23.530 | −16.255 | 93.625 | 1.00 | 18.67 | C |
| ATOM | 4645 | CG2 | ILE | B | 274 | 21.719 | −15.291 | 96.095 | 1.00 | 19.32 | C |
| ATOM | 4646 | C | ILE | B | 274 | 19.989 | −17.193 | 97.176 | 1.00 | 20.64 | C |
| ATOM | 4647 | O | ILE | B | 274 | 20.231 | −16.901 | 98.340 | 1.00 | 20.78 | O |
| ATOM | 4659 | N | SER | B | 275 | 18.776 | −17.089 | 96.655 | 1.00 | 21.52 | N |
| ATOM | 4660 | CA | SER | B | 275 | 17.635 | −16.620 | 97.416 | 1.00 | 22.24 | C |
| ATOM | 4661 | CB | SER | B | 275 | 16.448 | −16.396 | 96.481 | 1.00 | 22.89 | C |
| ATOM | 4662 | OG | SER | B | 275 | 15.230 | −16.441 | 97.210 | 1.00 | 25.58 | O |
| ATOM | 4663 | C | SER | B | 275 | 17.250 | −17.606 | 98.511 | 1.00 | 22.16 | C |
| ATOM | 4664 | O | SER | B | 275 | 16.795 | −17.202 | 99.586 | 1.00 | 22.60 | O |
| ATOM | 4670 | N | ILE | B | 276 | 17.437 | −18.891 | 98.228 | 1.00 | 21.61 | N |
| ATOM | 4671 | CA | ILE | B | 276 | 17.204 | −19.946 | 99.192 | 1.00 | 21.64 | C |
| ATOM | 4672 | CB | ILE | B | 276 | 17.191 | −21.330 | 98.492 | 1.00 | 21.18 | C |
| ATOM | 4673 | CG1 | ILE | B | 276 | 15.855 | −21.514 | 97.784 | 1.00 | 21.72 | C |
| ATOM | 4674 | CD1 | ILE | B | 276 | 15.844 | −22.615 | 96.736 | 1.00 | 21.24 | C |
| ATOM | 4675 | CG2 | ILE | B | 276 | 17.480 | −22.455 | 99.487 | 1.00 | 19.73 | C |
| ATOM | 4676 | C | ILE | B | 276 | 18.230 | −19.908 | 100.309 | 1.00 | 21.79 | C |
| ATOM | 4677 | O | ILE | B | 276 | 17.887 | −20.111 | 101.463 | 1.00 | 22.32 | O |
| ATOM | 4689 | N | VAL | B | 277 | 19.487 | −19.644 | 99.974 | 1.00 | 21.96 | N |
| ATOM | 4690 | CA | VAL | B | 277 | 20.521 | −19.464 | 100.984 | 1.00 | 21.75 | C |
| ATOM | 4691 | CB | VAL | B | 277 | 21.927 | −19.313 | 100.353 | 1.00 | 21.45 | C |
| ATOM | 4692 | CG1 | VAL | B | 277 | 22.969 | −18.930 | 101.394 | 1.00 | 21.31 | C |
| ATOM | 4693 | CG2 | VAL | B | 277 | 22.336 | −20.600 | 99.703 | 1.00 | 21.22 | C |
| ATOM | 4694 | C | VAL | B | 277 | 20.213 | −18.258 | 101.884 | 1.00 | 22.23 | C |
| ATOM | 4695 | O | VAL | B | 277 | 20.414 | −18.335 | 103.088 | 1.00 | 21.91 | O |
| ATOM | 4705 | N | ASP | B | 278 | 19.752 | −17.148 | 101.306 | 1.00 | 22.32 | N |
| ATOM | 4706 | CA | ASP | B | 278 | 19.381 | −15.988 | 102.110 | 1.00 | 22.49 | C |
| ATOM | 4707 | CB | ASP | B | 278 | 19.058 | −14.753 | 101.235 | 1.00 | 22.89 | C |
| ATOM | 4708 | CG | ASP | B | 278 | 18.665 | −13.519 | 102.076 | 1.00 | 25.45 | C |
| ATOM | 4709 | OD1 | ASP | B | 278 | 17.465 | −13.139 | 102.107 | 1.00 | 28.79 | O |
| ATOM | 4710 | OD2 | ASP | B | 278 | 19.487 | −12.868 | 102.763 | 1.00 | 27.03 | O |
| ATOM | 4711 | C | ASP | B | 278 | 18.215 | −16.334 | 103.058 | 1.00 | 21.82 | C |
| ATOM | 4712 | O | ASP | B | 278 | 18.211 | −15.898 | 104.202 | 1.00 | 21.62 | O |
| ATOM | 4717 | N | TRP | B | 279 | 17.251 | −17.130 | 102.600 | 1.00 | 21.23 | N |
| ATOM | 4718 | CA | TRP | B | 279 | 16.153 | −17.602 | 103.471 | 1.00 | 20.95 | C |
| ATOM | 4719 | CB | TRP | B | 279 | 15.183 | −18.473 | 102.685 | 1.00 | 20.76 | C |
| ATOM | 4720 | CG | TRP | B | 279 | 14.348 | −19.383 | 103.519 | 1.00 | 19.54 | C |
| ATOM | 4721 | CD1 | TRP | B | 279 | 13.234 | −19.053 | 104.227 | 1.00 | 19.44 | C |
| ATOM | 4722 | NE1 | TRP | B | 279 | 12.725 | −20.165 | 104.857 | 1.00 | 19.44 | N |
| ATOM | 4723 | CE2 | TRP | B | 279 | 13.504 | −21.249 | 104.550 | 1.00 | 19.20 | C |
| ATOM | 4724 | CD2 | TRP | B | 279 | 14.535 | −20.796 | 103.705 | 1.00 | 20.07 | C |
| ATOM | 4725 | CE3 | TRP | B | 279 | 15.472 | −21.730 | 103.237 | 1.00 | 19.96 | C |
| ATOM | 4726 | CZ3 | TRP | B | 279 | 15.357 | −23.060 | 103.647 | 1.00 | 21.50 | C |
| ATOM | 4727 | CH2 | TRP | B | 279 | 14.318 | −23.474 | 104.488 | 1.00 | 20.86 | C |
| ATOM | 4728 | CZ2 | TRP | B | 279 | 13.380 | −22.594 | 104.939 | 1.00 | 20.44 | C |
| ATOM | 4729 | C | TRP | B | 279 | 16.659 | −18.402 | 104.680 | 1.00 | 21.06 | C |
| ATOM | 4730 | O | TRP | B | 279 | 16.266 | −18.162 | 105.817 | 1.00 | 20.73 | O |
| ATOM | 4741 | N | ALA | B | 280 | 17.545 | −19.348 | 104.406 | 1.00 | 21.12 | N |
| ATOM | 4742 | CA | ALA | B | 280 | 18.095 | −20.227 | 105.415 | 1.00 | 21.36 | C |
| ATOM | 4743 | CB | ALA | B | 280 | 18.945 | −21.287 | 104.764 | 1.00 | 21.57 | C |
| ATOM | 4744 | C | ALA | B | 280 | 18.909 | −19.494 | 106.469 | 1.00 | 21.17 | C |
| ATOM | 4745 | O | ALA | B | 280 | 18.727 | −19.749 | 107.655 | 1.00 | 20.74 | O |
| ATOM | 4751 | N | ARG | B | 281 | 19.789 | −18.581 | 106.055 | 1.00 | 21.41 | N |
| ATOM | 4752 | CA | ARG | B | 281 | 20.649 | −17.893 | 107.009 | 1.00 | 21.77 | C |
| ATOM | 4753 | CB | ARG | B | 281 | 21.742 | −17.063 | 106.324 | 1.00 | 22.12 | C |
| ATOM | 4754 | CG | ARG | B | 281 | 21.255 | −15.934 | 105.466 | 1.00 | 23.17 | C |
| ATOM | 4755 | CD | ARG | B | 281 | 22.092 | −14.642 | 105.554 | 1.00 | 24.68 | C |
| ATOM | 4756 | NE | ARG | B | 281 | 21.245 | −13.492 | 105.210 | 1.00 | 26.13 | N |
| ATOM | 4757 | CZ | ARG | B | 281 | 20.990 | −12.446 | 106.002 | 1.00 | 27.24 | C |
| ATOM | 4758 | NH1 | ARG | B | 281 | 21.552 | −12.346 | 107.201 | 1.00 | 27.57 | N |
| ATOM | 4759 | NH2 | ARG | B | 281 | 20.178 | −11.472 | 105.575 | 1.00 | 27.88 | N |
| ATOM | 4760 | C | ARG | B | 281 | 19.855 | −17.058 | 108.017 | 1.00 | 21.86 | C |
| ATOM | 4761 | O | ARG | B | 281 | 20.345 | −16.785 | 109.108 | 1.00 | 21.98 | O |
| ATOM | 4775 | N | ARG | B | 282 | 18.629 | −16.687 | 107.652 | 1.00 | 22.24 | N |
| ATOM | 4776 | CA | ARG | B | 282 | 17.715 | −15.969 | 108.539 | 1.00 | 22.38 | C |
| ATOM | 4777 | CB | ARG | B | 282 | 16.739 | −15.152 | 107.712 | 1.00 | 22.31 | C |
| ATOM | 4778 | CG | ARG | B | 282 | 17.384 | −14.070 | 106.899 | 1.00 | 22.57 | C |
| ATOM | 4779 | CD | ARG | B | 282 | 16.384 | −13.389 | 106.002 | 1.00 | 22.90 | C |
| ATOM | 4780 | NE | ARG | B | 282 | 15.679 | −12.326 | 106.730 | 1.00 | 24.48 | N |
| ATOM | 4781 | CZ | ARG | B | 282 | 14.354 | −12.149 | 106.763 | 1.00 | 23.61 | C |
| ATOM | 4782 | NH1 | ARG | B | 282 | 13.545 | −12.975 | 106.093 | 1.00 | 23.21 | N |
| ATOM | 4783 | NH2 | ARG | B | 282 | 13.842 | −11.120 | 107.457 | 1.00 | 20.80 | N |
| ATOM | 4784 | C | ARG | B | 282 | 16.884 | −16.867 | 109.433 | 1.00 | 22.43 | C |
| ATOM | 4785 | O | ARG | B | 282 | 16.316 | −16.380 | 110.414 | 1.00 | 22.84 | O |
| ATOM | 4799 | N | CYS | B | 283 | 16.769 | −18.144 | 109.081 | 1.00 | 22.29 | N |
| ATOM | 4800 | CA | CYS | B | 283 | 15.891 | −19.070 | 109.821 | 1.00 | 22.73 | C |
| ATOM | 4801 | CB | CYS | B | 283 | 15.754 | −20.437 | 109.130 | 1.00 | 22.69 | C |
| ATOM | 4802 | SG | CYS | B | 283 | 14.571 | −20.498 | 107.793 | 1.00 | 22.22 | S |
| ATOM | 4803 | C | CYS | B | 283 | 16.334 | −19.327 | 111.238 | 1.00 | 22.77 | C |
| ATOM | 4804 | O | CYS | B | 283 | 17.513 | −19.262 | 111.559 | 1.00 | 22.82 | O |
| ATOM | 4810 | N | MET | B | 284 | 15.348 | −19.653 | 112.062 | 1.00 | 23.22 | N |
| ATOM | 4811 | CA | MET | B | 284 | 15.550 | −20.064 | 113.442 | 1.00 | 23.63 | C |
| ATOM | 4812 | CB | MET | B | 284 | 14.214 | −20.406 | 114.116 | 1.00 | 24.07 | C |
| ATOM | 4813 | CG | MET | B | 284 | 13.198 | −19.271 | 114.111 | 1.00 | 24.96 | C |
| ATOM | 4814 | SD | MET | B | 284 | 12.335 | −19.065 | 112.528 | 1.00 | 27.35 | S |
| ATOM | 4815 | CE | MET | B | 284 | 11.934 | −20.767 | 112.068 | 1.00 | 25.81 | C |
| ATOM | 4816 | C | MET | B | 284 | 16.428 | −21.299 | 113.480 | 1.00 | 23.32 | C |
| ATOM | 4817 | O | MET | B | 284 | 16.368 | −22.130 | 112.582 | 1.00 | 23.31 | O |
| ATOM | 4827 | N | VAL | B | 285 | 17.234 | −21.389 | 114.533 | 1.00 | 23.26 | N |
| ATOM | 4828 | CA | VAL | B | 285 | 18.192 | −22.469 | 114.740 | 1.00 | 22.92 | C |
| ATOM | 4829 | CB | VAL | B | 285 | 17.526 | −23.874 | 114.731 | 1.00 | 22.83 | C |
| ATOM | 4830 | CG1 | VAL | B | 285 | 18.584 | −24.953 | 114.959 | 1.00 | 23.23 | C |
| ATOM | 4831 | CG2 | VAL | B | 285 | 16.482 | −23.945 | 115.810 | 1.00 | 22.39 | C |
| ATOM | 4832 | C | VAL | B | 285 | 19.368 | −22.379 | 113.761 | 1.00 | 22.38 | C |
| ATOM | 4833 | O | VAL | B | 285 | 20.503 | −22.176 | 114.178 | 1.00 | 22.60 | O |
| ATOM | 4843 | N | PHE | B | 286 | 19.104 | −22.526 | 112.473 | 1.00 | 21.64 | N |
| ATOM | 4844 | CA | PHE | B | 286 | 20.137 | −22.356 | 111.466 | 1.00 | 21.39 | C |
| ATOM | 4845 | CB | PHE | B | 286 | 19.502 | −22.327 | 110.064 | 1.00 | 21.00 | C |
| ATOM | 4846 | CG | PHE | B | 286 | 20.496 | −22.517 | 108.951 | 1.00 | 20.08 | C |
| ATOM | 4847 | CD1 | PHE | B | 286 | 20.719 | −23.772 | 108.413 | 1.00 | 18.72 | C |
| ATOM | 4848 | CE1 | PHE | B | 286 | 21.633 | −23.941 | 107.412 | 1.00 | 19.77 | C |
| ATOM | 4849 | CZ | PHE | B | 286 | 22.363 | −22.833 | 106.935 | 1.00 | 19.66 | C |
| ATOM | 4850 | CE2 | PHE | B | 286 | 22.161 | −21.587 | 107.476 | 1.00 | 17.57 | C |
| ATOM | 4851 | CD2 | PHE | B | 286 | 21.237 | −21.430 | 108.471 | 1.00 | 18.42 | C |
| ATOM | 4852 | C | PHE | B | 286 | 21.021 | −21.104 | 111.712 | 1.00 | 21.71 | C |
| ATOM | 4853 | O | PHE | B | 286 | 22.249 | −21.170 | 111.566 | 1.00 | 21.83 | O |
| ATOM | 4863 | N | LYS | B | 287 | 20.410 | −19.983 | 112.098 | 1.00 | 21.75 | N |
| ATOM | 4864 | CA | LYS | B | 287 | 21.158 | −18.730 | 112.339 | 1.00 | 21.95 | C |
| ATOM | 4865 | CB | LYS | B | 287 | 20.196 | −17.533 | 112.410 | 1.00 | 21.82 | C |
| ATOM | 4866 | CG | LYS | B | 287 | 19.233 | −17.553 | 113.570 | 1.00 | 21.85 | C |
| ATOM | 4867 | CD | LYS | B | 287 | 18.315 | −16.356 | 113.550 | 1.00 | 22.26 | C |
| ATOM | 4868 | CE | LYS | B | 287 | 17.148 | −16.564 | 114.520 | 1.00 | 23.12 | C |
| ATOM | 4869 | NZ | LYS | B | 287 | 16.770 | −15.317 | 115.262 | 1.00 | 23.87 | N |
| ATOM | 4870 | C | LYS | B | 287 | 22.080 | −18.746 | 113.578 | 1.00 | 22.07 | C |
| ATOM | 4871 | O | LYS | B | 287 | 22.992 | −17.913 | 113.715 | 1.00 | 21.88 | O |
| ATOM | 4885 | N | GLU | B | 288 | 21.834 | −19.672 | 114.494 | 1.00 | 22.31 | N |
| ATOM | 4886 | CA | GLU | B | 288 | 22.687 | −19.804 | 115.670 | 1.00 | 22.72 | C |
| ATOM | 4887 | CB | GLU | B | 288 | 21.867 | −20.164 | 116.924 | 1.00 | 22.76 | C |
| ATOM | 4888 | CG | GLU | B | 288 | 20.543 | −19.421 | 117.084 | 1.00 | 23.78 | C |
| ATOM | 4889 | CD | GLU | B | 288 | 20.697 | −17.952 | 117.465 | 1.00 | 26.00 | C |
| ATOM | 4890 | OE1 | GLU | B | 288 | 19.703 | −17.201 | 117.307 | 1.00 | 27.85 | O |
| ATOM | 4891 | OE2 | GLU | B | 288 | 21.787 | −17.533 | 117.939 | 1.00 | 27.27 | O |
| ATOM | 4892 | C | GLU | B | 288 | 23.773 | −20.851 | 115.420 | 1.00 | 22.64 | C |
| ATOM | 4893 | O | GLU | B | 288 | 24.701 | −20.981 | 116.211 | 1.00 | 22.97 | O |
| ATOM | 4900 | N | LEU | B | 289 | 23.637 | −21.616 | 114.343 | 1.00 | 22.85 | N |
| ATOM | 4901 | CA | LEU | B | 289 | 24.675 | −22.560 | 113.944 | 1.00 | 23.32 | C |
| ATOM | 4902 | CB | LEU | B | 289 | 24.175 | −23.509 | 112.841 | 1.00 | 23.61 | C |
| ATOM | 4903 | CG | LEU | B | 289 | 23.522 | −24.858 | 113.206 | 1.00 | 24.52 | C |
| ATOM | 4904 | CD1 | LEU | B | 289 | 22.450 | −24.738 | 114.256 | 1.00 | 24.97 | C |
| ATOM | 4905 | CD2 | LEU | B | 289 | 22.943 | −25.531 | 111.972 | 1.00 | 24.62 | C |
| ATOM | 4906 | C | LEU | B | 289 | 25.866 | −21.763 | 113.425 | 1.00 | 23.52 | C |
| ATOM | 4907 | O | LEU | B | 289 | 25.686 | −20.714 | 112.788 | 1.00 | 23.37 | O |
| ATOM | 4919 | N | GLU | B | 290 | 27.081 | −22.237 | 113.691 | 1.00 | 23.70 | N |
| ATOM | 4920 | CA | GLU | B | 290 | 28.245 | −21.591 | 113.086 | 1.00 | 24.18 | C |
| ATOM | 4921 | CB | GLU | B | 290 | 29.555 | −21.840 | 113.858 | 1.00 | 24.72 | C |
| ATOM | 4922 | CG | GLU | B | 290 | 30.035 | −23.283 | 113.924 | 1.00 | 26.12 | C |
| ATOM | 4923 | CD | GLU | B | 290 | 30.590 | −23.657 | 115.289 | 1.00 | 28.89 | C |
| ATOM | 4924 | OE1 | GLU | B | 290 | 31.449 | −24.570 | 115.342 | 1.00 | 31.77 | O |
| ATOM | 4925 | OE2 | GLU | B | 290 | 30.176 | −23.046 | 116.311 | 1.00 | 29.77 | O |
| ATOM | 4926 | C | GLU | B | 290 | 28.348 | −21.996 | 111.624 | 1.00 | 23.92 | C |
| ATOM | 4927 | O | GLU | B | 290 | 27.724 | −22.973 | 111.190 | 1.00 | 24.08 | O |
| ATOM | 4934 | N | VAL | B | 291 | 29.115 | −21.219 | 110.866 | 1.00 | 23.42 | N |
| ATOM | 4935 | CA | VAL | B | 291 | 29.116 | −21.321 | 109.412 | 1.00 | 23.20 | C |
| ATOM | 4936 | CB | VAL | B | 291 | 30.079 | −20.259 | 108.740 | 1.00 | 23.54 | C |
| ATOM | 4937 | CG1 | VAL | B | 291 | 30.321 | −20.553 | 107.244 | 1.00 | 23.73 | C |
| ATOM | 4938 | CG2 | VAL | B | 291 | 29.520 | −18.840 | 108.903 | 1.00 | 24.07 | C |
| ATOM | 4939 | C | VAL | B | 291 | 29.437 | −22.750 | 108.968 | 1.00 | 22.55 | C |
| ATOM | 4940 | O | VAL | B | 291 | 28.892 | −23.209 | 107.980 | 1.00 | 22.73 | O |
| ATOM | 4950 | N | ALA | B | 292 | 30.296 | −23.466 | 109.692 | 1.00 | 21.85 | N |
| ATOM | 4951 | CA | ALA | B | 292 | 30.643 | −24.828 | 109.278 | 1.00 | 21.49 | C |
| ATOM | 4952 | CB | ALA | B | 292 | 31.696 | −25.434 | 110.207 | 1.00 | 21.37 | C |
| ATOM | 4953 | C | ALA | B | 292 | 29.396 | −25.735 | 109.178 | 1.00 | 21.05 | C |
| ATOM | 4954 | O | ALA | B | 292 | 29.201 | −26.416 | 108.178 | 1.00 | 20.32 | O |
| ATOM | 4960 | N | ASP | B | 293 | 28.557 | −25.726 | 110.205 | 1.00 | 20.81 | N |
| ATOM | 4961 | CA | ASP | B | 293 | 27.364 | −26.563 | 110.198 | 1.00 | 21.03 | C |
| ATOM | 4962 | CB | ASP | B | 293 | 26.698 | −26.582 | 111.572 | 1.00 | 20.84 | C |
| ATOM | 4963 | CG | ASP | B | 293 | 27.249 | −27.657 | 112.467 | 1.00 | 20.66 | C |
| ATOM | 4964 | OD1 | ASP | B | 293 | 27.789 | −28.661 | 111.947 | 1.00 | 19.06 | O |
| ATOM | 4965 | OD2 | ASP | B | 293 | 27.184 | −27.564 | 113.712 | 1.00 | 20.70 | O |
| ATOM | 4966 | C | ASP | B | 293 | 26.357 | −26.102 | 109.166 | 1.00 | 21.01 | C |
| ATOM | 4967 | O | ASP | B | 293 | 25.696 | −26.917 | 108.529 | 1.00 | 21.20 | O |
| ATOM | 4972 | N | GLN | B | 294 | 26.234 | −24.788 | 109.025 | 1.00 | 21.06 | N |
| ATOM | 4973 | CA | GLN | B | 294 | 25.370 | −24.186 | 108.023 | 1.00 | 20.59 | C |
| ATOM | 4974 | CB | GLN | B | 294 | 25.508 | −22.676 | 108.092 | 1.00 | 20.60 | C |
| ATOM | 4975 | CG | GLN | B | 294 | 24.788 | −22.053 | 109.257 | 1.00 | 20.21 | C |
| ATOM | 4976 | CD | GLN | B | 294 | 24.765 | −20.553 | 109.166 | 1.00 | 20.67 | C |
| ATOM | 4977 | OE1 | GLN | B | 294 | 25.562 | −19.996 | 108.260 | 1.00 | 22.24 | O |
| ATOM | 4978 | NE2 | GLN | B | 294 | 24.076 | −19.899 | 109.939 | 1.00 | 21.98 | N |
| ATOM | 4979 | C | GLN | B | 294 | 25.724 | −24.672 | 106.618 | 1.00 | 20.45 | C |
| ATOM | 4980 | O | GLN | B | 294 | 24.841 | −24.955 | 105.821 | 1.00 | 20.19 | O |
| ATOM | 4989 | N | MET | B | 295 | 27.015 | −24.791 | 106.326 | 1.00 | 20.27 | N |
| ATOM | 4990 | CA | MET | B | 295 | 27.462 | −25.238 | 105.010 | 1.00 | 20.39 | C |
| ATOM | 4991 | CB | MET | B | 295 | 28.955 | −24.963 | 104.846 | 1.00 | 20.36 | C |
| ATOM | 4992 | CG | MET | B | 295 | 29.294 | −23.480 | 104.824 | 1.00 | 21.58 | C |
| ATOM | 4993 | SD | MET | B | 295 | 31.075 | −23.126 | 104.729 | 1.00 | 26.19 | S |
| ATOM | 4994 | CE | MET | B | 295 | 31.281 | −23.322 | 103.006 | 1.00 | 26.02 | C |
| ATOM | 4995 | C | MET | B | 295 | 27.162 | −26.718 | 104.779 | 1.00 | 20.13 | C |
| ATOM | 4996 | O | MET | B | 295 | 26.797 | −27.119 | 103.695 | 1.00 | 20.41 | O |
| ATOM | 5006 | N | THR | B | 296 | 27.310 | −27.511 | 105.825 | 1.00 | 20.34 | N |
| ATOM | 5007 | CA | THR | B | 296 | 27.135 | −28.953 | 105.776 | 1.00 | 20.32 | C |
| ATOM | 5008 | CB | THR | B | 296 | 27.548 | −29.572 | 107.164 | 1.00 | 20.31 | C |
| ATOM | 5009 | OG1 | THR | B | 296 | 28.966 | −29.446 | 107.372 | 1.00 | 19.78 | O |
| ATOM | 5010 | CG2 | THR | B | 296 | 27.290 | −31.077 | 107.229 | 1.00 | 19.90 | C |
| ATOM | 5011 | C | THR | B | 296 | 25.694 | −29.303 | 105.441 | 1.00 | 20.29 | C |
| ATOM | 5012 | O | THR | B | 296 | 25.442 | −30.164 | 104.603 | 1.00 | 20.47 | O |
| ATOM | 5020 | N | LEU | B | 297 | 24.756 | −28.635 | 106.111 | 1.00 | 20.31 | N |
| ATOM | 5021 | CA | LEU | B | 297 | 23.321 | −28.819 | 105.872 | 1.00 | 20.34 | C |
| ATOM | 5022 | CB | LEU | B | 297 | 22.519 | −27.933 | 106.812 | 1.00 | 20.37 | C |
| ATOM | 5023 | CG | LEU | B | 297 | 22.520 | −28.330 | 108.273 | 1.00 | 20.70 | C |
| ATOM | 5024 | CD1 | LEU | B | 297 | 21.731 | −27.291 | 109.069 | 1.00 | 21.32 | C |
| ATOM | 5025 | CD2 | LEU | B | 297 | 21.946 | −29.750 | 108.451 | 1.00 | 20.56 | C |
| ATOM | 5026 | C | LEU | B | 297 | 22.934 | −28.468 | 104.463 | 1.00 | 20.22 | C |
| ATOM | 5027 | O | LEU | B | 297 | 22.314 | −29.255 | 103.758 | 1.00 | 20.93 | O |
| ATOM | 5039 | N | LEU | B | 298 | 23.299 | −27.259 | 104.067 | 1.00 | 20.30 | N |
| ATOM | 5040 | CA | LEU | B | 298 | 23.092 | −26.780 | 102.713 | 1.00 | 19.83 | C |
| ATOM | 5041 | CB | LEU | B | 298 | 23.526 | −25.318 | 102.587 | 1.00 | 19.73 | C |
| ATOM | 5042 | CG | LEU | B | 298 | 22.615 | −24.275 | 103.243 | 1.00 | 19.57 | C |
| ATOM | 5043 | CD1 | LEU | B | 298 | 23.317 | −22.950 | 103.331 | 1.00 | 20.68 | C |
| ATOM | 5044 | CD2 | LEU | B | 298 | 21.279 | −24.124 | 102.520 | 1.00 | 19.62 | C |
| ATOM | 5045 | C | LEU | B | 298 | 23.792 | −27.627 | 101.659 | 1.00 | 20.04 | C |
| ATOM | 5046 | O | LEU | B | 298 | 23.277 | −27.773 | 100.584 | 1.00 | 19.76 | O |
| ATOM | 5058 | N | GLN | B | 299 | 24.951 | −28.200 | 101.964 | 1.00 | 21.20 | N |
| ATOM | 5059 | CA | GLN | B | 299 | 25.628 | −29.079 | 101.002 | 1.00 | 21.72 | C |
| ATOM | 5060 | CB | GLN | B | 299 | 27.059 | −29.402 | 101.446 | 1.00 | 22.32 | C |
| ATOM | 5061 | CG | GLN | B | 299 | 28.126 | −28.470 | 100.872 | 1.00 | 23.94 | C |
| ATOM | 5062 | CD | GLN | B | 299 | 29.454 | −28.516 | 101.632 | 1.00 | 27.45 | C |
| ATOM | 5063 | OE1 | GLN | B | 299 | 30.321 | −27.665 | 101.418 | 1.00 | 28.07 | O |
| ATOM | 5064 | NE2 | GLN | B | 299 | 29.618 | −29.509 | 102.515 | 1.00 | 30.84 | N |
| ATOM | 5065 | C | GLN | B | 299 | 24.819 | −30.362 | 100.804 | 1.00 | 21.88 | C |
| ATOM | 5066 | O | GLN | B | 299 | 24.829 | −30.940 | 99.730 | 1.00 | 22.34 | O |
| ATOM | 5075 | N | ASN | B | 300 | 24.094 | −30.777 | 101.835 | 1.00 | 21.96 | N |
| ATOM | 5076 | CA | ASN | B | 300 | 23.299 | −32.000 | 101.800 | 1.00 | 22.38 | C |
| ATOM | 5077 | CB | ASN | B | 300 | 23.065 | −32.499 | 103.238 | 1.00 | 23.00 | C |
| ATOM | 5078 | CG | ASN | B | 300 | 22.013 | −33.608 | 103.328 | 1.00 | 25.15 | C |
| ATOM | 5079 | OD1 | ASN | B | 300 | 22.296 | −34.765 | 102.982 | 1.00 | 28.54 | O |
| ATOM | 5080 | ND2 | ASN | B | 300 | 20.795 | −33.265 | 103.808 | 1.00 | 24.65 | N |
| ATOM | 5081 | C | ASN | B | 300 | 21.959 | −31.870 | 101.076 | 1.00 | 21.83 | C |
| ATOM | 5082 | O | ASN | B | 300 | 21.467 | −32.846 | 100.514 | 1.00 | 21.54 | O |
| ATOM | 5089 | N | CYS | B | 301 | 21.387 | −30.670 | 101.070 | 1.00 | 21.46 | N |
| ATOM | 5090 | CA | CYS | B | 301 | 20.003 | −30.471 | 100.633 | 1.00 | 21.54 | C |
| ATOM | 5091 | CB | CYS | B | 301 | 19.126 | −30.080 | 101.840 | 1.00 | 21.45 | C |
| ATOM | 5092 | SG | CYS | B | 301 | 19.477 | −28.428 | 102.440 | 1.00 | 22.49 | S |
| ATOM | 5093 | C | CYS | B | 301 | 19.804 | −29.438 | 99.540 | 1.00 | 20.51 | C |
| ATOM | 5094 | O | CYS | B | 301 | 18.676 | −29.092 | 99.262 | 1.00 | 20.64 | O |
| ATOM | 5100 | N | TRP | B | 302 | 20.882 | −28.934 | 98.935 | 1.00 | 20.27 | N |
| ATOM | 5101 | CA | TRP | B | 302 | 20.759 | −27.853 | 97.958 | 1.00 | 19.67 | C |
| ATOM | 5102 | CB | TRP | B | 302 | 22.137 | −27.364 | 97.458 | 1.00 | 19.77 | C |
| ATOM | 5103 | CG | TRP | B | 302 | 22.926 | −28.383 | 96.721 | 1.00 | 18.99 | C |
| ATOM | 5104 | CD1 | TRP | B | 302 | 23.750 | −29.312 | 97.264 | 1.00 | 17.57 | C |
| ATOM | 5105 | NE1 | TRP | B | 302 | 24.266 | −30.114 | 96.279 | 1.00 | 16.02 | N |
| ATOM | 5106 | CE2 | TRP | B | 302 | 23.784 | −29.710 | 95.069 | 1.00 | 15.34 | C |
| ATOM | 5107 | CD2 | TRP | B | 302 | 22.934 | −28.614 | 95.308 | 1.00 | 16.64 | C |
| ATOM | 5108 | CE3 | TRP | B | 302 | 22.287 | −28.021 | 94.217 | 1.00 | 15.05 | C |
| ATOM | 5109 | CZ3 | TRP | B | 302 | 22.521 | −28.523 | 92.956 | 1.00 | 14.93 | C |
| ATOM | 5110 | CH2 | TRP | B | 302 | 23.377 | −29.610 | 92.754 | 1.00 | 16.76 | C |
| ATOM | 5111 | CZ2 | TRP | B | 302 | 24.025 | −30.211 | 93.796 | 1.00 | 16.30 | C |
| ATOM | 5112 | C | TRP | B | 302 | 19.818 | −28.234 | 96.786 | 1.00 | 19.44 | C |
| ATOM | 5113 | O | TRP | B | 302 | 18.952 | −27.462 | 96.414 | 1.00 | 18.38 | O |
| ATOM | 5124 | N | SER | B | 303 | 19.982 | −29.439 | 96.248 | 1.00 | 19.51 | N |
| ATOM | 5125 | CA | SER | B | 303 | 19.195 | −29.907 | 95.120 | 1.00 | 19.82 | C |
| ATOM | 5126 | CB | SER | B | 303 | 19.869 | −31.113 | 94.460 | 1.00 | 19.56 | C |
| ATOM | 5127 | OG | SER | B | 303 | 20.086 | −32.137 | 95.387 | 1.00 | 20.85 | O |
| ATOM | 5128 | C | SER | B | 303 | 17.723 | −30.220 | 95.490 | 1.00 | 19.83 | C |
| ATOM | 5129 | O | SER | B | 303 | 16.809 | −29.987 | 94.696 | 1.00 | 18.13 | O |
| ATOM | 5135 | N | GLU | B | 304 | 17.514 | −30.711 | 96.705 | 1.00 | 19.98 | N |
| ATOM | 5136 | CA | GLU | B | 304 | 16.165 | −30.950 | 97.213 | 1.00 | 20.80 | C |
| ATOM | 5137 | CB | GLU | B | 304 | 16.239 | −31.727 | 98.532 | 1.00 | 21.27 | C |
| ATOM | 5138 | CG | GLU | B | 304 | 16.641 | −33.205 | 98.395 | 1.00 | 24.23 | C |
| ATOM | 5139 | CD | GLU | B | 304 | 18.159 | −33.451 | 98.398 | 1.00 | 29.84 | C |
| ATOM | 5140 | OE1 | GLU | B | 304 | 18.928 | −32.457 | 98.230 | 1.00 | 33.46 | O |
| ATOM | 5141 | OE2 | GLU | B | 304 | 18.602 | −34.641 | 98.555 | 1.00 | 31.69 | O |
| ATOM | 5142 | C | GLU | B | 304 | 15.345 | −29.645 | 97.360 | 1.00 | 20.45 | C |
| ATOM | 5143 | O | GLU | B | 304 | 14.178 | −29.579 | 97.002 | 1.00 | 19.98 | O |
| ATOM | 5150 | N | LEU | B | 305 | 15.981 | −28.595 | 97.841 | 1.00 | 20.78 | N |
| ATOM | 5151 | CA | LEU | B | 305 | 15.314 | −27.326 | 98.038 | 1.00 | 21.70 | C |
| ATOM | 5152 | CB | LEU | B | 305 | 16.228 | −26.379 | 98.829 | 1.00 | 22.34 | C |
| ATOM | 5153 | CG | LEU | B | 305 | 15.980 | −26.075 | 100.314 | 1.00 | 23.87 | C |
| ATOM | 5154 | CD1 | LEU | B | 305 | 14.969 | −26.950 | 101.059 | 1.00 | 23.15 | C |
| ATOM | 5155 | CD2 | LEU | B | 305 | 17.342 | −26.100 | 101.007 | 1.00 | 25.21 | C |
| ATOM | 5156 | C | LEU | B | 305 | 14.938 | −26.677 | 96.721 | 1.00 | 21.77 | C |
| ATOM | 5157 | O | LEU | B | 305 | 13.867 | −26.110 | 96.597 | 1.00 | 21.41 | O |
| ATOM | 5169 | N | LEU | B | 306 | 15.841 | −26.755 | 95.743 | 1.00 | 22.26 | N |
| ATOM | 5170 | CA | LEU | B | 306 | 15.574 | −26.265 | 94.392 | 1.00 | 22.10 | C |
| ATOM | 5171 | CB | LEU | B | 306 | 16.820 | −26.376 | 93.542 | 1.00 | 22.42 | C |
| ATOM | 5172 | CG | LEU | B | 306 | 17.771 | −25.239 | 93.824 | 1.00 | 23.36 | C |
| ATOM | 5173 | CD1 | LEU | B | 306 | 19.197 | −25.638 | 93.387 | 1.00 | 24.98 | C |
| ATOM | 5174 | CD2 | LEU | B | 306 | 17.262 | −23.966 | 93.156 | 1.00 | 22.31 | C |
| ATOM | 5175 | C | LEU | B | 306 | 14.455 | −27.008 | 93.695 | 1.00 | 21.82 | C |
| ATOM | 5176 | O | LEU | B | 306 | 13.597 | −26.397 | 93.086 | 1.00 | 21.87 | O |
| ATOM | 5188 | N | VAL | B | 307 | 14.470 | −28.327 | 93.797 | 1.00 | 21.88 | N |
| ATOM | 5189 | CA | VAL | B | 307 | 13.410 | −29.166 | 93.241 | 1.00 | 22.05 | C |
| ATOM | 5190 | CB | VAL | B | 307 | 13.761 | −30.673 | 93.432 | 1.00 | 21.75 | C |
| ATOM | 5191 | CG1 | VAL | B | 307 | 12.565 | −31.595 | 93.128 | 1.00 | 22.21 | C |
| ATOM | 5192 | CG2 | VAL | B | 307 | 14.925 | −31.043 | 92.562 | 1.00 | 21.95 | C |
| ATOM | 5193 | C | VAL | B | 307 | 12.044 | −28.831 | 93.841 | 1.00 | 21.41 | C |
| ATOM | 5194 | O | VAL | B | 307 | 11.078 | −28.601 | 93.148 | 1.00 | 21.08 | O |
| ATOM | 5204 | N | PHE | B | 308 | 12.001 | −28.799 | 95.153 | 1.00 | 21.79 | N |
| ATOM | 5205 | CA | PHE | B | 308 | 10.781 | −28.546 | 95.897 | 1.00 | 22.11 | C |
| ATOM | 5206 | CB | PHE | B | 308 | 11.055 | −28.760 | 97.390 | 1.00 | 22.05 | C |
| ATOM | 5207 | CG | PHE | B | 308 | 9.820 | −28.877 | 98.235 | 1.00 | 22.51 | C |
| ATOM | 5208 | CD1 | PHE | B | 308 | 9.644 | −28.060 | 99.349 | 1.00 | 22.64 | C |
| ATOM | 5209 | CE1 | PHE | B | 308 | 8.510 | −28.180 | 100.166 | 1.00 | 22.90 | C |
| ATOM | 5210 | CZ | PHE | B | 308 | 7.557 | −29.127 | 99.874 | 1.00 | 22.77 | C |
| ATOM | 5211 | CE2 | PHE | B | 308 | 7.724 | −29.962 | 98.767 | 1.00 | 22.96 | C |
| ATOM | 5212 | CD2 | PHE | B | 308 | 8.863 | −29.832 | 97.955 | 1.00 | 22.95 | C |
| ATOM | 5213 | C | PHE | B | 308 | 10.273 | −27.140 | 95.630 | 1.00 | 21.44 | C |
| ATOM | 5214 | O | PHE | B | 308 | 9.088 | −26.912 | 95.555 | 1.00 | 21.90 | O |
| ATOM | 5224 | N | ASP | B | 309 | 11.187 | −26.200 | 95.474 | 1.00 | 21.39 | N |
| ATOM | 5225 | CA | ASP | B | 309 | 10.834 | −24.848 | 95.057 | 1.00 | 21.05 | C |
| ATOM | 5226 | CB | ASP | B | 309 | 12.088 | −23.970 | 95.004 | 1.00 | 21.59 | C |
| ATOM | 5227 | CG | ASP | B | 309 | 11.802 | −22.578 | 94.493 | 1.00 | 21.57 | C |
| ATOM | 5228 | OD1 | ASP | B | 309 | 12.098 | −22.287 | 93.329 | 1.00 | 21.09 | O |
| ATOM | 5229 | OD2 | ASP | B | 309 | 11.259 | −21.712 | 95.180 | 1.00 | 26.55 | O |
| ATOM | 5230 | C | ASP | B | 309 | 10.133 | −24.854 | 93.709 | 1.00 | 20.83 | C |
| ATOM | 5231 | O | ASP | B | 309 | 9.072 | −24.274 | 93.564 | 1.00 | 20.35 | O |
| ATOM | 5236 | N | HIS | B | 310 | 10.721 | −25.541 | 92.739 | 1.00 | 21.00 | N |
| ATOM | 5237 | CA | HIS | B | 310 | 10.103 | −25.747 | 91.429 | 1.00 | 21.25 | C |
| ATOM | 5238 | CB | HIS | B | 310 | 11.040 | −26.574 | 90.522 | 1.00 | 21.15 | C |
| ATOM | 5239 | CG | HIS | B | 310 | 10.402 | −27.069 | 89.257 | 1.00 | 19.60 | C |
| ATOM | 5240 | ND1 | HIS | B | 310 | 10.144 | −26.249 | 88.182 | 1.00 | 19.91 | N |
| ATOM | 5241 | CE1 | HIS | B | 310 | 9.579 | −26.956 | 87.219 | 1.00 | 19.44 | C |
| ATOM | 5242 | NE2 | HIS | B | 310 | 9.466 | −28.205 | 87.630 | 1.00 | 18.08 | N |
| ATOM | 5243 | CD2 | HIS | B | 310 | 9.971 | −28.302 | 88.899 | 1.00 | 18.53 | C |
| ATOM | 5244 | C | HIS | B | 310 | 8.755 | −26.436 | 91.536 | 1.00 | 21.32 | C |
| ATOM | 5245 | O | HIS | B | 310 | 7.797 | −26.013 | 90.910 | 1.00 | 21.75 | O |
| ATOM | 5254 | N | ILE | B | 311 | 8.695 | −27.486 | 92.343 | 1.00 | 21.88 | N |
| ATOM | 5255 | CA | ILE | B | 311 | 7.499 | −28.300 | 92.498 | 1.00 | 21.49 | C |
| ATOM | 5256 | CB | ILE | B | 311 | 7.763 | −29.461 | 93.484 | 1.00 | 22.24 | C |
| ATOM | 5257 | CG1 | ILE | B | 311 | 8.726 | −30.493 | 92.901 | 1.00 | 22.50 | C |
| ATOM | 5258 | CD1 | ILE | B | 311 | 8.122 | −31.324 | 91.859 | 1.00 | 24.07 | C |
| ATOM | 5259 | CG2 | ILE | B | 311 | 6.479 | −30.147 | 93.903 | 1.00 | 22.13 | C |
| ATOM | 5260 | C | ILE | B | 311 | 6.390 | −27.424 | 93.037 | 1.00 | 21.50 | C |
| ATOM | 5261 | O | ILE | B | 311 | 5.269 | −27.464 | 92.550 | 1.00 | 20.80 | O |
| ATOM | 5273 | N | TYR | B | 312 | 6.699 | −26.613 | 94.043 | 1.00 | 21.46 | N |
| ATOM | 5274 | CA | TYR | B | 312 | 5.654 | −25.836 | 94.689 | 1.00 | 21.05 | C |
| ATOM | 5275 | CB | TYR | B | 312 | 6.105 | −25.266 | 96.045 | 1.00 | 21.15 | C |
| ATOM | 5276 | CG | TYR | B | 312 | 4.966 | −24.603 | 96.809 | 1.00 | 21.33 | C |
| ATOM | 5277 | CD1 | TYR | B | 312 | 3.875 | −25.340 | 97.234 | 1.00 | 20.83 | C |
| ATOM | 5278 | CE1 | TYR | B | 312 | 2.833 | −24.748 | 97.909 | 1.00 | 20.35 | C |
| ATOM | 5279 | CZ | TYR | B | 312 | 2.849 | −23.412 | 98.141 | 1.00 | 20.63 | C |
| ATOM | 5280 | OH | TYR | B | 312 | 1.775 | −22.811 | 98.800 | 1.00 | 21.74 | O |
| ATOM | 5281 | CE2 | TYR | B | 312 | 3.907 | −22.647 | 97.692 | 1.00 | 20.69 | C |
| ATOM | 5282 | CD2 | TYR | B | 312 | 4.947 | −23.239 | 97.035 | 1.00 | 20.68 | C |
| ATOM | 5283 | C | TYR | B | 312 | 5.163 | −24.722 | 93.786 | 1.00 | 20.94 | C |
| ATOM | 5284 | O | TYR | B | 312 | 4.004 | −24.386 | 93.846 | 1.00 | 21.75 | O |
| ATOM | 5294 | N | ARG | B | 313 | 6.039 | −24.153 | 92.955 | 1.00 | 20.48 | N |
| ATOM | 5295 | CA | ARG | B | 313 | 5.649 | −23.153 | 91.980 | 1.00 | 20.05 | C |
| ATOM | 5296 | CB | ARG | B | 313 | 6.876 | −22.680 | 91.207 | 1.00 | 20.14 | C |
| ATOM | 5297 | CG | ARG | B | 313 | 6.609 | −21.665 | 90.143 | 1.00 | 19.58 | C |
| ATOM | 5298 | CD | ARG | B | 313 | 7.840 | −20.897 | 89.669 | 1.00 | 20.44 | C |
| ATOM | 5299 | NE | ARG | B | 313 | 8.596 | −20.313 | 90.778 | 1.00 | 20.86 | N |
| ATOM | 5300 | CZ | ARG | B | 313 | 9.670 | −20.859 | 91.365 | 1.00 | 18.45 | C |
| ATOM | 5301 | NH1 | ARG | B | 313 | 10.201 | −21.977 | 90.946 | 1.00 | 19.23 | N |
| ATOM | 5302 | NH2 | ARG | B | 313 | 10.238 | −20.243 | 92.361 | 1.00 | 19.38 | N |
| ATOM | 5303 | C | ARG | B | 313 | 4.628 | −23.712 | 91.018 | 1.00 | 19.80 | C |
| ATOM | 5304 | O | ARG | B | 313 | 3.760 | −22.983 | 90.546 | 1.00 | 19.89 | O |
| ATOM | 5318 | N | GLN | B | 314 | 4.745 | −25.007 | 90.733 | 1.00 | 20.11 | N |
| ATOM | 5319 | CA | GLN | B | 314 | 3.838 | −25.721 | 89.833 | 1.00 | 20.74 | C |
| ATOM | 5320 | CB | GLN | B | 314 | 4.444 | −27.044 | 89.332 | 1.00 | 20.80 | C |
| ATOM | 5321 | CG | GLN | B | 314 | 5.752 | −27.018 | 88.510 | 1.00 | 20.83 | C |
| ATOM | 5322 | CD | GLN | B | 314 | 5.951 | −25.764 | 87.709 | 1.00 | 20.62 | C |
| ATOM | 5323 | OE1 | GLN | B | 314 | 5.058 | −25.504 | 86.771 | 1.00 | 23.09 | O |
| ATOM | 5324 | NE2 | GLN | B | 314 | 6.905 | −25.027 | 87.951 | 1.00 | 20.26 | N |
| ATOM | 5325 | C | GLN | B | 314 | 2.504 | −26.035 | 90.496 | 1.00 | 20.93 | C |
| ATOM | 5326 | O | GLN | B | 314 | 1.461 | −26.074 | 89.834 | 1.00 | 21.31 | O |
| ATOM | 5335 | N | VAL | B | 315 | 2.545 | −26.287 | 91.792 | 1.00 | 21.47 | N |
| ATOM | 5336 | CA | VAL | B | 315 | 1.339 | −26.477 | 92.588 | 1.00 | 21.86 | C |
| ATOM | 5337 | CB | VAL | B | 315 | 1.695 | −26.874 | 94.042 | 1.00 | 21.90 | C |
| ATOM | 5338 | CG1 | VAL | B | 315 | 0.452 | −26.870 | 94.944 | 1.00 | 21.57 | C |
| ATOM | 5339 | CG2 | VAL | B | 315 | 2.389 | −28.246 | 94.093 | 1.00 | 22.69 | C |
| ATOM | 5340 | C | VAL | B | 315 | 0.515 | −25.184 | 92.587 | 1.00 | 22.33 | C |
| ATOM | 5341 | O | VAL | B | 315 | −0.684 | −25.206 | 92.394 | 1.00 | 22.71 | O |
| ATOM | 5351 | N | GLN | B | 316 | 1.178 | −24.059 | 92.816 | 1.00 | 22.87 | N |
| ATOM | 5352 | CA | GLN | B | 316 | 0.579 | −22.747 | 92.676 | 1.00 | 23.16 | C |
| ATOM | 5353 | CB | GLN | B | 316 | 1.617 | −21.714 | 93.064 | 1.00 | 23.70 | C |
| ATOM | 5354 | CG | GLN | B | 316 | 1.939 | −21.660 | 94.544 | 1.00 | 25.96 | C |
| ATOM | 5355 | CD | GLN | B | 316 | 2.938 | −20.545 | 94.866 | 1.00 | 29.62 | C |
| ATOM | 5356 | OE1 | GLN | B | 316 | 4.023 | −20.466 | 94.251 | 1.00 | 29.77 | O |
| ATOM | 5357 | NE2 | GLN | B | 316 | 2.569 | −19.668 | 95.810 | 1.00 | 31.49 | N |
| ATOM | 5358 | C | GLN | B | 316 | 0.045 | −22.405 | 91.264 | 1.00 | 22.88 | C |
| ATOM | 5359 | O | GLN | B | 316 | −0.976 | −21.764 | 91.139 | 1.00 | 22.62 | O |
| ATOM | 5368 | N | HIS | B | 317 | 0.774 | −22.819 | 90.228 | 1.00 | 22.96 | N |
| ATOM | 5369 | CA | HIS | B | 317 | 0.440 | −22.619 | 88.800 | 1.00 | 22.71 | C |
| ATOM | 5370 | CB | HIS | B | 317 | 1.624 | −23.147 | 87.977 | 1.00 | 22.82 | C |
| ATOM | 5371 | CG | HIS | B | 317 | 1.516 | −22.925 | 86.505 | 1.00 | 22.98 | C |
| ATOM | 5372 | ND1 | HIS | B | 317 | 1.188 | −21.829 | 85.787 | 1.00 | 23.02 | N |
| ATOM | 5373 | CE1 | HIS | B | 317 | 1.298 | −22.170 | 84.461 | 1.00 | 20.97 | C |
| ATOM | 5374 | NE2 | HIS | B | 317 | 1.688 | −23.427 | 84.368 | 1.00 | 22.88 | N |
| ATOM | 5375 | CD2 | HIS | B | 317 | 1.833 | −23.905 | 85.588 | 1.00 | 21.70 | C |
| ATOM | 5376 | C | HIS | B | 317 | −0.840 | −23.364 | 88.389 | 1.00 | 22.46 | C |
| ATOM | 5377 | O | HIS | B | 317 | −1.677 | −22.812 | 87.707 | 1.00 | 22.08 | O |
| ATOM | 5386 | N | GLY | B | 318 | −0.971 | −24.614 | 88.827 | 1.00 | 22.55 | N |
| ATOM | 5387 | CA | GLY | B | 318 | −2.170 | −25.409 | 88.643 | 1.00 | 22.69 | C |
| ATOM | 5388 | C | GLY | B | 318 | −2.602 | −25.526 | 87.190 | 1.00 | 23.03 | C |
| ATOM | 5389 | O | GLY | B | 318 | −3.760 | −25.291 | 86.873 | 1.00 | 22.58 | O |
| ATOM | 5393 | N | LYS | B | 319 | −1.650 | −25.824 | 86.305 | 1.00 | 23.49 | N |
| ATOM | 5394 | CA | LYS | B | 319 | −1.922 | −26.083 | 84.891 | 1.00 | 23.20 | C |
| ATOM | 5395 | CB | LYS | B | 319 | −1.556 | −24.894 | 84.002 | 1.00 | 23.43 | C |
| ATOM | 5396 | CG | LYS | B | 319 | −2.234 | −23.561 | 84.313 | 1.00 | 24.64 | C |
| ATOM | 5397 | CD | LYS | B | 319 | −2.180 | −22.590 | 83.084 | 1.00 | 26.59 | C |
| ATOM | 5398 | CE | LYS | B | 319 | −2.124 | −21.076 | 83.466 | 1.00 | 27.34 | C |
| ATOM | 5399 | NZ | LYS | B | 319 | −3.265 | −20.259 | 82.933 | 1.00 | 28.68 | N |
| ATOM | 5400 | C | LYS | B | 319 | −1.038 | −27.232 | 84.500 | 1.00 | 23.02 | C |
| ATOM | 5401 | O | LYS | B | 319 | 0.167 | −27.115 | 84.545 | 1.00 | 23.76 | O |
| ATOM | 5415 | N | GLU | B | 320 | −1.631 | −28.344 | 84.110 | 1.00 | 22.79 | N |
| ATOM | 5416 | CA | GLU | B | 320 | −0.873 | −29.524 | 83.708 | 1.00 | 22.90 | C |
| ATOM | 5417 | CB | GLU | B | 320 | −1.824 | −30.707 | 83.582 | 1.00 | 23.42 | C |
| ATOM | 5418 | CG | GLU | B | 320 | −2.188 | −31.317 | 84.910 | 1.00 | 25.32 | C |
| ATOM | 5419 | CD | GLU | B | 320 | −2.927 | −32.596 | 84.700 | 1.00 | 27.91 | C |
| ATOM | 5420 | OE1 | GLU | B | 320 | −4.130 | −32.648 | 85.061 | 1.00 | 31.19 | O |
| ATOM | 5421 | OE2 | GLU | B | 320 | −2.303 | −33.526 | 84.126 | 1.00 | 30.61 | O |
| ATOM | 5422 | C | GLU | B | 320 | −0.103 | −29.411 | 82.381 | 1.00 | 21.87 | C |
| ATOM | 5423 | O | GLU | B | 320 | 0.910 | −30.085 | 82.217 | 1.00 | 22.12 | O |
| ATOM | 5430 | N | GLY | B | 321 | −0.581 | −28.581 | 81.456 | 1.00 | 20.73 | N |
| ATOM | 5431 | CA | GLY | B | 321 | 0.006 | −28.441 | 80.132 | 1.00 | 20.44 | C |
| ATOM | 5432 | C | GLY | B | 321 | 1.298 | −27.635 | 80.010 | 1.00 | 20.18 | C |
| ATOM | 5433 | O | GLY | B | 321 | 1.944 | −27.678 | 78.965 | 1.00 | 20.14 | O |
| ATOM | 5437 | N | SER | B | 322 | 1.668 | −26.893 | 81.046 | 1.00 | 20.00 | N |
| ATOM | 5438 | CA | SER | B | 322 | 2.902 | −26.123 | 81.027 | 1.00 | 20.11 | C |
| ATOM | 5439 | CB | SER | B | 322 | 2.679 | −24.693 | 80.513 | 1.00 | 19.76 | C |
| ATOM | 5440 | OG | SER | B | 322 | 1.809 | −23.969 | 81.355 | 1.00 | 20.88 | O |
| ATOM | 5441 | C | SER | B | 322 | 3.541 | −26.076 | 82.393 | 1.00 | 19.98 | C |
| ATOM | 5442 | O | SER | B | 322 | 2.881 | −26.298 | 83.405 | 1.00 | 20.62 | O |
| ATOM | 5448 | N | ILE | B | 323 | 4.842 | −25.802 | 82.398 | 1.00 | 19.67 | N |
| ATOM | 5449 | CA | ILE | B | 323 | 5.581 | −25.499 | 83.607 | 1.00 | 19.58 | C |
| ATOM | 5450 | CB | ILE | B | 323 | 6.847 | −26.406 | 83.733 | 1.00 | 20.06 | C |
| ATOM | 5451 | CG1 | ILE | B | 323 | 7.831 | −26.185 | 82.573 | 1.00 | 20.96 | C |
| ATOM | 5452 | CD1 | ILE | B | 323 | 9.245 | −26.517 | 82.893 | 1.00 | 21.82 | C |
| ATOM | 5453 | CG2 | ILE | B | 323 | 6.403 | −27.889 | 83.816 | 1.00 | 19.02 | C |
| ATOM | 5454 | C | ILE | B | 323 | 5.877 | −24.004 | 83.705 | 1.00 | 18.66 | C |
| ATOM | 5455 | O | ILE | B | 323 | 6.011 | −23.320 | 82.723 | 1.00 | 17.59 | O |
| ATOM | 5467 | N | LEU | B | 324 | 5.871 | −23.505 | 84.925 | 1.00 | 19.02 | N |
| ATOM | 5468 | CA | LEU | B | 324 | 6.091 | −22.093 | 85.222 | 1.00 | 18.94 | C |
| ATOM | 5469 | CB | LEU | B | 324 | 4.984 | −21.593 | 86.147 | 1.00 | 18.81 | C |
| ATOM | 5470 | CG | LEU | B | 324 | 4.998 | −20.106 | 86.509 | 1.00 | 18.54 | C |
| ATOM | 5471 | CD1 | LEU | B | 324 | 4.604 | −19.260 | 85.355 | 1.00 | 19.16 | C |
| ATOM | 5472 | CD2 | LEU | B | 324 | 4.074 | −19.889 | 87.670 | 1.00 | 18.76 | C |
| ATOM | 5473 | C | LEU | B | 324 | 7.472 | −21.918 | 85.867 | 1.00 | 18.41 | C |
| ATOM | 5474 | O | LEU | B | 324 | 7.808 | −22.590 | 86.815 | 1.00 | 18.18 | O |
| ATOM | 5486 | N | LEU | B | 325 | 8.274 | −21.025 | 85.314 | 1.00 | 19.31 | N |
| ATOM | 5487 | CA | LEU | B | 325 | 9.587 | −20.713 | 85.866 | 1.00 | 20.23 | C |
| ATOM | 5488 | CB | LEU | B | 325 | 10.613 | −20.481 | 84.759 | 1.00 | 20.48 | C |
| ATOM | 5489 | CG | LEU | B | 325 | 10.568 | −21.506 | 83.604 | 1.00 | 20.83 | C |
| ATOM | 5490 | CD1 | LEU | B | 325 | 11.655 | −21.277 | 82.628 | 1.00 | 21.68 | C |
| ATOM | 5491 | CD2 | LEU | B | 325 | 10.651 | −22.913 | 84.097 | 1.00 | 21.82 | C |
| ATOM | 5492 | C | LEU | B | 325 | 9.472 | −19.519 | 86.791 | 1.00 | 20.95 | C |
| ATOM | 5493 | O | LEU | B | 325 | 8.467 | −18.817 | 86.804 | 1.00 | 20.70 | O |
| ATOM | 5505 | N | VAL | B | 326 | 10.490 | −19.329 | 87.612 | 1.00 | 22.23 | N |
| ATOM | 5506 | CA | VAL | B | 326 | 10.458 | −18.278 | 88.626 | 1.00 | 23.07 | C |
| ATOM | 5507 | CB | VAL | B | 326 | 11.611 | −18.411 | 89.673 | 1.00 | 23.46 | C |
| ATOM | 5508 | CG1 | VAL | B | 326 | 13.003 | −18.179 | 89.026 | 1.00 | 24.79 | C |
| ATOM | 5509 | CG2 | VAL | B | 326 | 11.397 | −17.435 | 90.840 | 1.00 | 23.97 | C |
| ATOM | 5510 | C | VAL | B | 326 | 10.509 | −16.897 | 87.994 | 1.00 | 23.05 | C |
| ATOM | 5511 | O | VAL | B | 326 | 10.133 | −15.918 | 88.641 | 1.00 | 23.38 | O |
| ATOM | 5521 | N | THR | B | 327 | 10.984 | −16.828 | 86.754 | 1.00 | 22.52 | N |
| ATOM | 5522 | CA | THR | B | 327 | 10.954 | −15.594 | 85.986 | 1.00 | 22.89 | C |
| ATOM | 5523 | CB | THR | B | 327 | 11.784 | −15.715 | 84.699 | 1.00 | 23.11 | C |
| ATOM | 5524 | OG1 | THR | B | 327 | 11.386 | −16.890 | 83.968 | 1.00 | 22.59 | O |
| ATOM | 5525 | CG2 | THR | B | 327 | 13.277 | −15.870 | 85.036 | 1.00 | 23.51 | C |
| ATOM | 5526 | C | THR | B | 327 | 9.562 | −15.189 | 85.557 | 1.00 | 22.77 | C |
| ATOM | 5527 | O | THR | B | 327 | 9.361 | −14.042 | 85.182 | 1.00 | 22.67 | O |
| ATOM | 5535 | N | GLY | B | 328 | 8.639 | −16.144 | 85.552 | 1.00 | 22.68 | N |
| ATOM | 5536 | CA | GLY | B | 328 | 7.284 | −15.923 | 85.129 | 1.00 | 22.64 | C |
| ATOM | 5537 | C | GLY | B | 328 | 7.031 | −16.505 | 83.761 | 1.00 | 23.07 | C |
| ATOM | 5538 | O | GLY | B | 328 | 5.893 | −16.531 | 83.302 | 1.00 | 23.49 | O |
| ATOM | 5542 | N | GLN | B | 329 | 8.069 | −16.971 | 83.086 | 1.00 | 23.52 | N |
| ATOM | 5543 | CA | GLN | B | 329 | 7.860 | −17.539 | 81.761 | 1.00 | 23.91 | C |
| ATOM | 5544 | CB | GLN | B | 329 | 9.094 | −17.351 | 80.842 | 1.00 | 24.51 | C |
| ATOM | 5545 | CG | GLN | B | 329 | 10.367 | −18.083 | 81.191 | 1.00 | 26.54 | C |
| ATOM | 5546 | CD | GLN | B | 329 | 11.668 | −17.273 | 80.852 | 1.00 | 30.05 | C |
| ATOM | 5547 | OE1 | GLN | B | 329 | 12.667 | −17.343 | 81.747 | 1.00 | 33.63 | O |
| ATOM | 5548 | NE2 | GLN | B | 329 | 11.755 | −16.609 | 79.804 | 1.00 | 30.94 | N |
| ATOM | 5549 | C | GLN | B | 329 | 7.337 | −18.986 | 81.814 | 1.00 | 22.90 | C |
| ATOM | 5550 | O | GLN | B | 329 | 7.708 | −19.764 | 82.676 | 1.00 | 22.66 | O |
| ATOM | 5559 | N | GLU | B | 330 | 6.424 | −19.287 | 80.894 | 1.00 | 22.31 | N |
| ATOM | 5560 | CA | GLU | B | 330 | 5.735 | −20.571 | 80.791 | 1.00 | 21.68 | C |
| ATOM | 5561 | CB | GLU | B | 330 | 4.264 | −20.339 | 80.435 | 1.00 | 21.82 | C |
| ATOM | 5562 | CG | GLU | B | 330 | 3.252 | −20.645 | 81.527 | 1.00 | 23.21 | C |
| ATOM | 5563 | CD | GLU | B | 330 | 1.930 | −19.910 | 81.338 | 1.00 | 24.77 | C |
| ATOM | 5564 | OE1 | GLU | B | 330 | 1.321 | −19.443 | 82.329 | 1.00 | 27.79 | O |
| ATOM | 5565 | OE2 | GLU | B | 330 | 1.483 | −19.769 | 80.195 | 1.00 | 26.18 | O |
| ATOM | 5566 | C | GLU | B | 330 | 6.378 | −21.386 | 79.678 | 1.00 | 20.95 | C |
| ATOM | 5567 | O | GLU | B | 330 | 6.739 | −20.845 | 78.636 | 1.00 | 21.08 | O |
| ATOM | 5574 | N | VAL | B | 331 | 6.523 | −22.684 | 79.890 | 1.00 | 19.80 | N |
| ATOM | 5575 | CA | VAL | B | 331 | 6.989 | −23.587 | 78.858 | 1.00 | 19.11 | C |
| ATOM | 5576 | CB | VAL | B | 331 | 8.383 | −24.160 | 79.178 | 1.00 | 19.27 | C |
| ATOM | 5577 | CG1 | VAL | B | 331 | 8.922 | −24.967 | 78.029 | 1.00 | 19.53 | C |
| ATOM | 5578 | CG2 | VAL | B | 331 | 9.366 | −23.044 | 79.543 | 1.00 | 19.22 | C |
| ATOM | 5579 | C | VAL | B | 331 | 6.005 | −24.725 | 78.758 | 1.00 | 18.56 | C |
| ATOM | 5580 | O | VAL | B | 331 | 5.759 | −25.432 | 79.707 | 1.00 | 17.52 | O |
| ATOM | 5590 | N | GLU | B | 332 | 5.449 | −24.904 | 77.578 | 1.00 | 19.06 | N |
| ATOM | 5591 | CA | GLU | B | 332 | 4.551 | −26.009 | 77.336 | 1.00 | 19.31 | C |
| ATOM | 5592 | CB | GLU | B | 332 | 3.840 | −25.869 | 75.995 | 1.00 | 19.80 | C |
| ATOM | 5593 | CG | GLU | B | 332 | 2.340 | −25.703 | 76.147 | 1.00 | 22.70 | C |
| ATOM | 5594 | CD | GLU | B | 332 | 1.886 | −24.280 | 75.955 | 1.00 | 26.13 | C |
| ATOM | 5595 | OE1 | GLU | B | 332 | 1.550 | −23.942 | 74.787 | 1.00 | 27.53 | O |
| ATOM | 5596 | OE2 | GLU | B | 332 | 1.851 | −23.514 | 76.960 | 1.00 | 28.01 | O |
| ATOM | 5597 | C | GLU | B | 332 | 5.310 | −27.297 | 77.390 | 1.00 | 18.70 | C |
| ATOM | 5598 | O | GLU | B | 332 | 6.478 | −27.356 | 77.066 | 1.00 | 17.93 | O |
| ATOM | 5605 | N | LEU | B | 333 | 4.618 | −28.326 | 77.829 | 1.00 | 18.85 | N |
| ATOM | 5606 | CA | LEU | B | 333 | 5.184 | −29.644 | 77.943 | 1.00 | 19.01 | C |
| ATOM | 5607 | CB | LEU | B | 333 | 4.239 | −30.574 | 78.724 | 1.00 | 19.51 | C |
| ATOM | 5608 | CG | LEU | B | 333 | 4.390 | −30.820 | 80.237 | 1.00 | 20.04 | C |
| ATOM | 5609 | CD1 | LEU | B | 333 | 5.841 | −30.774 | 80.667 | 1.00 | 21.65 | C |
| ATOM | 5610 | CD2 | LEU | B | 333 | 3.603 | −29.881 | 81.039 | 1.00 | 20.78 | C |
| ATOM | 5611 | C | LEU | B | 333 | 5.441 | −30.192 | 76.557 | 1.00 | 18.61 | C |
| ATOM | 5612 | O | LEU | B | 333 | 6.283 | −31.053 | 76.396 | 1.00 | 18.43 | O |
| ATOM | 5624 | N | THR | B | 334 | 4.709 | −29.694 | 75.561 | 1.00 | 18.86 | N |
| ATOM | 5625 | CA | THR | B | 334 | 4.971 | −30.047 | 74.166 | 1.00 | 18.83 | C |
| ATOM | 5626 | CB | THR | B | 334 | 3.900 | −29.487 | 73.189 | 1.00 | 18.90 | C |
| ATOM | 5627 | OG1 | THR | B | 334 | 3.645 | −28.106 | 73.470 | 1.00 | 20.09 | O |
| ATOM | 5628 | CG2 | THR | B | 334 | 2.559 | −30.159 | 73.366 | 1.00 | 18.44 | C |
| ATOM | 5629 | C | THR | B | 334 | 6.347 | −29.552 | 73.732 | 1.00 | 18.70 | C |
| ATOM | 5630 | O | THR | B | 334 | 7.040 | −30.216 | 72.968 | 1.00 | 18.50 | O |
| ATOM | 5638 | N | THR | B | 335 | 6.719 | −28.372 | 74.203 | 1.00 | 18.43 | N |
| ATOM | 5639 | CA | THR | B | 335 | 7.993 | −27.792 | 73.866 | 1.00 | 18.47 | C |
| ATOM | 5640 | CB | THR | B | 335 | 8.080 | −26.358 | 74.356 | 1.00 | 18.29 | C |
| ATOM | 5641 | OG1 | THR | B | 335 | 7.134 | −25.556 | 73.662 | 1.00 | 16.43 | O |
| ATOM | 5642 | CG2 | THR | B | 335 | 9.428 | −25.741 | 73.968 | 1.00 | 18.60 | C |
| ATOM | 5643 | C | THR | B | 335 | 9.110 | −28.611 | 74.466 | 1.00 | 19.38 | C |
| ATOM | 5644 | O | THR | B | 335 | 10.135 | −28.837 | 73.816 | 1.00 | 19.78 | O |
| ATOM | 5652 | N | VAL | B | 336 | 8.918 | −29.081 | 75.687 | 1.00 | 19.76 | N |
| ATOM | 5653 | CA | VAL | B | 336 | 9.937 | −29.918 | 76.284 | 1.00 | 20.75 | C |
| ATOM | 5654 | CB | VAL | B | 336 | 9.949 | −29.929 | 77.859 | 1.00 | 21.14 | C |
| ATOM | 5655 | CG1 | VAL | B | 336 | 8.935 | −28.983 | 78.467 | 1.00 | 22.56 | C |
| ATOM | 5656 | CG2 | VAL | B | 336 | 9.816 | −31.314 | 78.427 | 1.00 | 22.19 | C |
| ATOM | 5657 | C | VAL | B | 336 | 9.956 | −31.325 | 75.674 | 1.00 | 20.72 | C |
| ATOM | 5658 | O | VAL | B | 336 | 11.028 | −31.896 | 75.518 | 1.00 | 20.49 | O |
| ATOM | 5668 | N | ALA | B | 337 | 8.793 | −31.860 | 75.292 | 1.00 | 20.96 | N |
| ATOM | 5669 | CA | ALA | B | 337 | 8.729 | −33.199 | 74.689 | 1.00 | 21.04 | C |
| ATOM | 5670 | CB | ALA | B | 337 | 7.281 | −33.597 | 74.383 | 1.00 | 21.17 | C |
| ATOM | 5671 | C | ALA | B | 337 | 9.564 | −33.238 | 73.416 | 1.00 | 21.02 | C |
| ATOM | 5672 | O | ALA | B | 337 | 10.128 | −34.261 | 73.049 | 1.00 | 21.74 | O |
| ATOM | 5678 | N | THR | B | 338 | 9.683 | −32.086 | 72.783 | 1.00 | 20.62 | N |
| ATOM | 5679 | CA | THR | B | 338 | 10.217 | −31.968 | 71.454 | 1.00 | 20.13 | C |
| ATOM | 5680 | CB | THR | B | 338 | 9.172 | −31.145 | 70.678 | 1.00 | 20.15 | C |
| ATOM | 5681 | OG1 | THR | B | 338 | 8.789 | −31.854 | 69.499 | 1.00 | 20.52 | O |
| ATOM | 5682 | CG2 | THR | B | 338 | 9.675 | −29.800 | 70.231 | 1.00 | 19.18 | C |
| ATOM | 5683 | C | THR | B | 338 | 11.658 | −31.380 | 71.421 | 1.00 | 19.85 | C |
| ATOM | 5684 | O | THR | B | 338 | 12.463 | −31.765 | 70.581 | 1.00 | 19.91 | O |
| ATOM | 5692 | N | GLN | B | 339 | 11.987 | −30.498 | 72.362 | 1.00 | 19.44 | N |
| ATOM | 5693 | CA | GLN | B | 339 | 13.287 | −29.815 | 72.386 | 1.00 | 19.33 | C |
| ATOM | 5694 | CB | GLN | B | 339 | 13.064 | −28.315 | 72.643 | 1.00 | 19.34 | C |
| ATOM | 5695 | CG | GLN | B | 339 | 12.191 | −27.605 | 71.630 | 1.00 | 19.69 | C |
| ATOM | 5696 | CD | GLN | B | 339 | 12.613 | −27.836 | 70.188 | 1.00 | 19.13 | C |
| ATOM | 5697 | OE1 | GLN | B | 339 | 11.688 | −28.311 | 69.380 | 1.00 | 20.42 | O |
| ATOM | 5698 | NE2 | GLN | B | 339 | 13.758 | −27.576 | 69.809 | 1.00 | 18.11 | N |
| ATOM | 5699 | C | GLN | B | 339 | 14.308 | −30.361 | 73.414 | 1.00 | 18.84 | C |
| ATOM | 5700 | O | GLN | B | 339 | 15.504 | −30.179 | 73.269 | 1.00 | 18.94 | O |
| ATOM | 5709 | N | ALA | B | 340 | 13.823 | −31.003 | 74.461 | 1.00 | 19.15 | N |
| ATOM | 5710 | CA | ALA | B | 340 | 14.683 | −31.523 | 75.515 | 1.00 | 19.55 | C |
| ATOM | 5711 | CB | ALA | B | 340 | 13.956 | −31.519 | 76.845 | 1.00 | 19.34 | C |
| ATOM | 5712 | C | ALA | B | 340 | 15.121 | −32.932 | 75.179 | 1.00 | 19.63 | C |
| ATOM | 5713 | O | ALA | B | 340 | 14.483 | −33.615 | 74.386 | 1.00 | 19.73 | O |
| ATOM | 5719 | N | GLY | B | 341 | 16.214 | −33.363 | 75.791 | 1.00 | 20.00 | N |
| ATOM | 5720 | CA | GLY | B | 341 | 16.676 | −34.723 | 75.649 | 1.00 | 20.38 | C |
| ATOM | 5721 | C | GLY | B | 341 | 15.957 | −35.656 | 76.599 | 1.00 | 20.77 | C |
| ATOM | 5722 | O | GLY | B | 341 | 15.116 | −35.236 | 77.376 | 1.00 | 20.80 | O |
| ATOM | 5726 | N | SER | B | 342 | 16.326 | −36.930 | 76.513 | 1.00 | 21.87 | N |
| ATOM | 5727 | CA | SER | B | 342 | 15.827 | −38.023 | 77.348 | 1.00 | 22.21 | C |
| ATOM | 5728 | CB | SER | B | 342 | 16.716 | −39.254 | 77.163 | 1.00 | 21.97 | C |
| ATOM | 5729 | OG | SER | B | 342 | 16.513 | −39.828 | 75.909 | 1.00 | 23.35 | O |
| ATOM | 5730 | C | SER | B | 342 | 15.812 | −37.731 | 78.829 | 1.00 | 22.52 | C |
| ATOM | 5731 | O | SER | B | 342 | 14.778 | −37.878 | 79.477 | 1.00 | 22.89 | O |
| ATOM | 5737 | N | LEU | B | 343 | 16.982 | −37.384 | 79.360 | 1.00 | 22.84 | N |
| ATOM | 5738 | CA | LEU | B | 343 | 17.155 | −37.151 | 80.783 | 1.00 | 23.34 | C |
| ATOM | 5739 | CB | LEU | B | 343 | 18.622 | −36.791 | 81.102 | 1.00 | 23.95 | C |
| ATOM | 5740 | CG | LEU | B | 343 | 19.726 | −37.873 | 81.105 | 1.00 | 26.27 | C |
| ATOM | 5741 | CD1 | LEU | B | 343 | 19.174 | −39.261 | 81.461 | 1.00 | 28.04 | C |
| ATOM | 5742 | CD2 | LEU | B | 343 | 20.512 | −37.981 | 79.777 | 1.00 | 28.14 | C |
| ATOM | 5743 | C | LEU | B | 343 | 16.232 | −36.037 | 81.263 | 1.00 | 22.93 | C |
| ATOM | 5744 | O | LEU | B | 343 | 15.420 | −36.244 | 82.168 | 1.00 | 23.32 | O |
| ATOM | 5756 | N | LEU | B | 344 | 16.341 | −34.872 | 80.634 | 1.00 | 22.28 | N |
| ATOM | 5757 | CA | LEU | B | 344 | 15.598 | −33.696 | 81.053 | 1.00 | 22.08 | C |
| ATOM | 5758 | CB | LEU | B | 344 | 16.066 | −32.454 | 80.286 | 1.00 | 21.40 | C |
| ATOM | 5759 | CG | LEU | B | 344 | 15.341 | −31.183 | 80.709 | 1.00 | 21.75 | C |
| ATOM | 5760 | CD1 | LEU | B | 344 | 15.549 | −30.958 | 82.222 | 1.00 | 21.88 | C |
| ATOM | 5761 | CD2 | LEU | B | 344 | 15.755 | −29.958 | 79.897 | 1.00 | 21.56 | C |
| ATOM | 5762 | C | LEU | B | 344 | 14.084 | −33.886 | 80.886 | 1.00 | 22.46 | C |
| ATOM | 5763 | O | LEU | B | 344 | 13.303 | −33.521 | 81.759 | 1.00 | 22.54 | O |
| ATOM | 5775 | N | HIS | B | 345 | 13.685 | −34.424 | 79.747 | 1.00 | 22.89 | N |
| ATOM | 5776 | CA | HIS | B | 345 | 12.299 | −34.732 | 79.493 | 1.00 | 23.28 | C |
| ATOM | 5777 | CB | HIS | B | 345 | 12.161 | −35.460 | 78.161 | 1.00 | 23.38 | C |
| ATOM | 5778 | CG | HIS | B | 345 | 10.741 | −35.692 | 77.737 | 1.00 | 23.67 | C |
| ATOM | 5779 | ND1 | HIS | B | 345 | 10.181 | −36.660 | 76.974 | 1.00 | 23.76 | N |
| ATOM | 5780 | CE1 | HIS | B | 345 | 8.837 | −36.386 | 76.891 | 1.00 | 24.30 | C |
| ATOM | 5781 | NE2 | HIS | B | 345 | 8.583 | −35.287 | 77.573 | 1.00 | 23.65 | N |
| ATOM | 5782 | CD2 | HIS | B | 345 | 9.715 | −34.851 | 78.097 | 1.00 | 24.06 | C |
| ATOM | 5783 | C | HIS | B | 345 | 11.714 | −35.588 | 80.616 | 1.00 | 23.75 | C |
| ATOM | 5784 | O | HIS | B | 345 | 10.665 | −35.240 | 81.166 | 1.00 | 23.59 | O |
| ATOM | 5793 | N | SER | B | 346 | 12.403 | −36.685 | 80.948 | 1.00 | 23.97 | N |
| ATOM | 5794 | CA | SER | B | 346 | 11.962 | −37.627 | 81.982 | 1.00 | 24.68 | C |
| ATOM | 5795 | CB | SER | B | 346 | 12.893 | −38.856 | 82.032 | 1.00 | 25.22 | C |
| ATOM | 5796 | OG | SER | B | 346 | 13.070 | −39.328 | 83.373 | 1.00 | 27.97 | O |
| ATOM | 5797 | C | SER | B | 346 | 11.884 | −36.999 | 83.365 | 1.00 | 24.28 | C |
| ATOM | 5798 | O | SER | B | 346 | 10.986 | −37.258 | 84.127 | 1.00 | 24.46 | O |
| ATOM | 5804 | N | LEU | B | 347 | 12.846 | −36.162 | 83.677 | 1.00 | 24.40 | N |
| ATOM | 5805 | CA | LEU | B | 347 | 12.877 | −35.436 | 84.939 | 1.00 | 24.27 | C |
| ATOM | 5806 | CB | LEU | B | 347 | 14.182 | −34.655 | 84.962 | 1.00 | 23.88 | C |
| ATOM | 5807 | CG | LEU | B | 347 | 14.828 | −34.173 | 86.239 | 1.00 | 26.27 | C |
| ATOM | 5808 | CD1 | LEU | B | 347 | 14.833 | −35.213 | 87.387 | 1.00 | 26.65 | C |
| ATOM | 5809 | CD2 | LEU | B | 347 | 16.245 | −33.727 | 85.882 | 1.00 | 26.86 | C |
| ATOM | 5810 | C | LEU | B | 347 | 11.665 | −34.492 | 85.089 | 1.00 | 24.01 | C |
| ATOM | 5811 | O | LEU | B | 347 | 10.999 | −34.457 | 86.106 | 1.00 | 24.10 | O |
| ATOM | 5823 | N | VAL | B | 348 | 11.384 | −33.728 | 84.052 | 1.00 | 23.56 | N |
| ATOM | 5824 | CA | VAL | B | 348 | 10.302 | −32.772 | 84.085 | 1.00 | 23.17 | C |
| ATOM | 5825 | CB | VAL | B | 348 | 10.284 | −31.928 | 82.789 | 1.00 | 23.27 | C |
| ATOM | 5826 | CG1 | VAL | B | 348 | 9.008 | −31.093 | 82.667 | 1.00 | 23.23 | C |
| ATOM | 5827 | CG2 | VAL | B | 348 | 11.513 | −31.042 | 82.720 | 1.00 | 23.56 | C |
| ATOM | 5828 | C | VAL | B | 348 | 8.986 | −33.508 | 84.309 | 1.00 | 23.05 | C |
| ATOM | 5829 | O | VAL | B | 348 | 8.209 | −33.108 | 85.153 | 1.00 | 23.12 | O |
| ATOM | 5839 | N | LEU | B | 349 | 8.763 | −34.595 | 83.576 | 1.00 | 22.99 | N |
| ATOM | 5840 | CA | LEU | B | 349 | 7.536 | −35.397 | 83.710 | 1.00 | 23.25 | C |
| ATOM | 5841 | CB | LEU | B | 349 | 7.501 | −36.503 | 82.659 | 1.00 | 23.12 | C |
| ATOM | 5842 | CG | LEU | B | 349 | 6.752 | −36.233 | 81.360 | 1.00 | 24.30 | C |
| ATOM | 5843 | CD1 | LEU | B | 349 | 6.615 | −34.749 | 81.020 | 1.00 | 25.05 | C |
| ATOM | 5844 | CD2 | LEU | B | 349 | 7.424 | −37.015 | 80.217 | 1.00 | 25.07 | C |
| ATOM | 5845 | C | LEU | B | 349 | 7.331 | −36.029 | 85.086 | 1.00 | 23.43 | C |
| ATOM | 5846 | O | LEU | B | 349 | 6.208 | −36.078 | 85.573 | 1.00 | 23.00 | O |
| ATOM | 5858 | N | ARG | B | 350 | 8.422 | −36.501 | 85.692 | 1.00 | 23.93 | N |
| ATOM | 5859 | CA | ARG | B | 350 | 8.395 | −37.140 | 86.995 | 1.00 | 24.51 | C |
| ATOM | 5860 | CB | ARG | B | 350 | 9.756 | −37.766 | 87.342 | 1.00 | 25.37 | C |
| ATOM | 5861 | CG | ARG | B | 350 | 9.713 | −38.938 | 88.345 | 1.00 | 28.59 | C |
| ATOM | 5862 | CD | ARG | B | 350 | 10.758 | −40.042 | 88.015 | 1.00 | 33.51 | C |
| ATOM | 5863 | NE | ARG | B | 350 | 11.034 | −41.071 | 89.054 | 1.00 | 37.55 | N |
| ATOM | 5864 | CZ | ARG | B | 350 | 11.576 | −40.840 | 90.272 | 1.00 | 38.97 | C |
| ATOM | 5865 | NH1 | ARG | B | 350 | 11.878 | −39.603 | 90.669 | 1.00 | 39.59 | N |
| ATOM | 5866 | NH2 | ARG | B | 350 | 11.797 | −41.854 | 91.113 | 1.00 | 39.17 | N |
| ATOM | 5867 | C | ARG | B | 350 | 8.027 | −36.118 | 88.028 | 1.00 | 24.08 | C |
| ATOM | 5868 | O | ARG | B | 350 | 7.204 | −36.394 | 88.903 | 1.00 | 24.39 | O |
| ATOM | 5882 | N | ALA | B | 351 | 8.629 | −34.932 | 87.913 | 1.00 | 23.55 | N |
| ATOM | 5883 | CA | ALA | B | 351 | 8.323 | −33.802 | 88.785 | 1.00 | 22.95 | C |
| ATOM | 5884 | CB | ALA | B | 351 | 9.197 | −32.621 | 88.440 | 1.00 | 22.93 | C |
| ATOM | 5885 | C | ALA | B | 351 | 6.853 | −33.410 | 88.718 | 1.00 | 22.23 | C |
| ATOM | 5886 | O | ALA | B | 351 | 6.224 | −33.172 | 89.737 | 1.00 | 21.92 | O |
| ATOM | 5892 | N | GLN | B | 352 | 6.326 | −33.359 | 87.506 | 1.00 | 21.95 | N |
| ATOM | 5893 | CA | GLN | B | 352 | 4.924 | −33.032 | 87.262 | 1.00 | 21.67 | C |
| ATOM | 5894 | CB | GLN | B | 352 | 4.651 | −32.970 | 85.755 | 1.00 | 21.25 | C |
| ATOM | 5895 | CG | GLN | B | 352 | 5.097 | −31.711 | 85.044 | 1.00 | 21.07 | C |
| ATOM | 5896 | CD | GLN | B | 352 | 4.677 | −30.432 | 85.744 | 1.00 | 20.58 | C |
| ATOM | 5897 | OE1 | GLN | B | 352 | 5.571 | −29.907 | 86.555 | 1.00 | 20.58 | O |
| ATOM | 5898 | NE2 | GLN | B | 352 | 3.563 | −29.914 | 85.542 | 1.00 | 20.12 | N |
| ATOM | 5899 | C | GLN | B | 352 | 3.960 | −34.040 | 87.921 | 1.00 | 21.82 | C |
| ATOM | 5900 | O | GLN | B | 352 | 2.884 | −33.673 | 88.338 | 1.00 | 21.80 | O |
| ATOM | 5909 | N | GLU | B | 353 | 4.344 | −35.309 | 87.989 | 1.00 | 22.34 | N |
| ATOM | 5910 | CA | GLU | B | 353 | 3.566 | −36.319 | 88.697 | 1.00 | 22.67 | C |
| ATOM | 5911 | CB | GLU | B | 353 | 4.143 | −37.713 | 88.475 | 1.00 | 23.12 | C |
| ATOM | 5912 | CG | GLU | B | 353 | 4.103 | −38.213 | 87.045 | 1.00 | 23.86 | C |
| ATOM | 5913 | CD | GLU | B | 353 | 4.609 | −39.636 | 86.929 | 1.00 | 27.46 | C |
| ATOM | 5914 | OE1 | GLU | B | 353 | 5.790 | −39.899 | 87.297 | 1.00 | 29.56 | O |
| ATOM | 5915 | OE2 | GLU | B | 353 | 3.824 | −40.507 | 86.479 | 1.00 | 29.46 | O |
| ATOM | 5916 | C | GLU | B | 353 | 3.509 | −36.080 | 90.188 | 1.00 | 22.32 | C |
| ATOM | 5917 | O | GLU | B | 353 | 2.510 | −36.336 | 90.795 | 1.00 | 23.45 | O |
| ATOM | 5924 | N | LEU | B | 354 | 4.594 | −35.619 | 90.775 | 1.00 | 22.08 | N |
| ATOM | 5925 | CA | LEU | B | 354 | 4.625 | −35.260 | 92.180 | 1.00 | 22.11 | C |
| ATOM | 5926 | CB | LEU | B | 354 | 6.085 | −35.105 | 92.602 | 1.00 | 22.17 | C |
| ATOM | 5927 | CG | LEU | B | 354 | 6.391 | −34.597 | 94.005 | 1.00 | 23.43 | C |
| ATOM | 5928 | CD1 | LEU | B | 354 | 5.860 | −35.556 | 95.001 | 1.00 | 24.79 | C |
| ATOM | 5929 | CD2 | LEU | B | 354 | 7.861 | −34.445 | 94.224 | 1.00 | 25.27 | C |
| ATOM | 5930 | C | LEU | B | 354 | 3.826 | −33.972 | 92.482 | 1.00 | 21.79 | C |
| ATOM | 5931 | O | LEU | B | 354 | 3.291 | −33.797 | 93.573 | 1.00 | 20.59 | O |
| ATOM | 5943 | N | VAL | B | 355 | 3.784 | −33.066 | 91.511 | 1.00 | 21.88 | N |
| ATOM | 5944 | CA | VAL | B | 355 | 2.921 | −31.897 | 91.574 | 1.00 | 21.58 | C |
| ATOM | 5945 | CB | VAL | B | 355 | 3.154 | −30.977 | 90.350 | 1.00 | 21.33 | C |
| ATOM | 5946 | CG1 | VAL | B | 355 | 2.109 | −29.825 | 90.271 | 1.00 | 20.89 | C |
| ATOM | 5947 | CG2 | VAL | B | 355 | 4.529 | −30.388 | 90.425 | 1.00 | 21.73 | C |
| ATOM | 5948 | C | VAL | B | 355 | 1.467 | −32.353 | 91.677 | 1.00 | 21.27 | C |
| ATOM | 5949 | O | VAL | B | 355 | 0.688 | −31.797 | 92.430 | 1.00 | 20.90 | O |
| ATOM | 5959 | N | LEU | B | 356 | 1.126 | −33.399 | 90.949 | 1.00 | 21.33 | N |
| ATOM | 5960 | CA | LEU | B | 356 | −0.231 | −33.918 | 90.931 | 1.00 | 21.93 | C |
| ATOM | 5961 | CB | LEU | B | 356 | −0.380 | −34.934 | 89.799 | 1.00 | 22.39 | C |
| ATOM | 5962 | CG | LEU | B | 356 | −1.514 | −34.808 | 88.772 | 1.00 | 24.08 | C |
| ATOM | 5963 | CD1 | LEU | B | 356 | −1.837 | −33.364 | 88.291 | 1.00 | 24.27 | C |
| ATOM | 5964 | CD2 | LEU | B | 356 | −1.168 | −35.709 | 87.600 | 1.00 | 24.55 | C |
| ATOM | 5965 | C | LEU | B | 356 | −0.635 | −34.535 | 92.278 | 1.00 | 22.05 | C |
| ATOM | 5966 | O | LEU | B | 356 | −1.746 | −34.280 | 92.764 | 1.00 | 21.37 | O |
| ATOM | 5978 | N | GLN | B | 357 | 0.273 | −35.324 | 92.869 | 1.00 | 22.25 | N |
| ATOM | 5979 | CA | GLN | B | 357 | 0.149 | −35.840 | 94.231 | 1.00 | 22.55 | C |
| ATOM | 5980 | CB | GLN | B | 357 | 1.439 | −36.540 | 94.673 | 1.00 | 23.08 | C |
| ATOM | 5981 | CG | GLN | B | 357 | 1.778 | −37.817 | 94.002 | 1.00 | 26.83 | C |
| ATOM | 5982 | CD | GLN | B | 357 | 3.092 | −38.448 | 94.565 | 1.00 | 32.10 | C |
| ATOM | 5983 | OE1 | GLN | B | 357 | 3.253 | −38.591 | 95.801 | 1.00 | 35.37 | O |
| ATOM | 5984 | NE2 | GLN | B | 357 | 4.019 | −38.830 | 93.655 | 1.00 | 31.88 | N |
| ATOM | 5985 | C | GLN | B | 357 | −0.093 | −34.735 | 95.250 | 1.00 | 21.89 | C |
| ATOM | 5986 | O | GLN | B | 357 | −0.989 | −34.820 | 96.050 | 1.00 | 21.86 | O |
| ATOM | 5995 | N | LEU | B | 358 | 0.770 | −33.727 | 95.248 | 1.00 | 22.32 | N |
| ATOM | 5996 | CA | LEU | B | 358 | 0.676 | −32.613 | 96.184 | 1.00 | 22.25 | C |
| ATOM | 5997 | CB | LEU | B | 358 | 1.927 | −31.699 | 96.100 | 1.00 | 22.07 | C |
| ATOM | 5998 | CG | LEU | B | 358 | 3.197 | −32.384 | 96.640 | 1.00 | 23.22 | C |
| ATOM | 5999 | CD1 | LEU | B | 358 | 4.486 | −31.681 | 96.197 | 1.00 | 24.57 | C |
| ATOM | 6000 | CD2 | LEU | B | 358 | 3.185 | −32.503 | 98.168 | 1.00 | 23.31 | C |
| ATOM | 6001 | C | LEU | B | 358 | −0.639 | −31.840 | 96.029 | 1.00 | 21.90 | C |
| ATOM | 6002 | O | LEU | B | 358 | −1.201 | −31.409 | 97.020 | 1.00 | 21.81 | O |
| ATOM | 6014 | N | LEU | B | 359 | −1.148 | −31.703 | 94.808 | 1.00 | 22.03 | N |
| ATOM | 6015 | CA | LEU | B | 359 | −2.484 | −31.100 | 94.578 | 1.00 | 21.79 | C |
| ATOM | 6016 | CB | LEU | B | 359 | −2.752 | −30.812 | 93.088 | 1.00 | 21.49 | C |
| ATOM | 6017 | CG | LEU | B | 359 | −1.950 | −29.654 | 92.483 | 1.00 | 21.57 | C |
| ATOM | 6018 | CD1 | LEU | B | 359 | −1.958 | −29.728 | 90.975 | 1.00 | 22.48 | C |
| ATOM | 6019 | CD2 | LEU | B | 359 | −2.477 | −28.317 | 92.947 | 1.00 | 21.25 | C |
| ATOM | 6020 | C | LEU | B | 359 | −3.604 | −31.988 | 95.120 | 1.00 | 21.66 | C |
| ATOM | 6021 | O | LEU | B | 359 | −4.583 | −31.491 | 95.672 | 1.00 | 21.05 | O |
| ATOM | 6033 | N | ALA | B | 360 | −3.437 | −33.296 | 94.968 | 1.00 | 21.68 | N |
| ATOM | 6034 | CA | ALA | B | 360 | −4.397 | −34.250 | 95.478 | 1.00 | 22.04 | C |
| ATOM | 6035 | CB | ALA | B | 360 | −4.157 | −35.648 | 94.858 | 1.00 | 22.02 | C |
| ATOM | 6036 | C | ALA | B | 360 | −4.407 | −34.292 | 97.016 | 1.00 | 22.38 | C |
| ATOM | 6037 | O | ALA | B | 360 | −5.460 | −34.442 | 97.613 | 1.00 | 22.99 | O |
| ATOM | 6043 | N | LEU | B | 361 | −3.255 | −34.108 | 97.650 | 1.00 | 22.87 | N |
| ATOM | 6044 | CA | LEU | B | 361 | −3.144 | −34.041 | 99.116 | 1.00 | 23.18 | C |
| ATOM | 6045 | CB | LEU | B | 361 | −1.707 | −34.358 | 99.551 | 1.00 | 23.45 | C |
| ATOM | 6046 | CG | LEU | B | 361 | −1.172 | −35.738 | 99.213 | 1.00 | 23.32 | C |
| ATOM | 6047 | CD1 | LEU | B | 361 | 0.324 | −35.801 | 99.442 | 1.00 | 22.44 | C |
| ATOM | 6048 | CD2 | LEU | B | 361 | −1.924 | −36.771 | 100.065 | 1.00 | 23.84 | C |
| ATOM | 6049 | C | LEU | B | 361 | −3.456 | −32.649 | 99.659 | 1.00 | 23.57 | C |
| ATOM | 6050 | O | LEU | B | 361 | −3.393 | −32.427 | 100.865 | 1.00 | 23.93 | O |
| ATOM | 6062 | N | GLN | B | 362 | −3.726 | −31.702 | 98.769 | 1.00 | 23.71 | N |
| ATOM | 6063 | CA | GLN | B | 362 | −4.104 | −30.343 | 99.153 | 1.00 | 23.59 | C |
| ATOM | 6064 | CB | GLN | B | 362 | −5.407 | −30.323 | 99.965 | 1.00 | 23.92 | C |
| ATOM | 6065 | CG | GLN | B | 362 | −6.619 | −30.595 | 99.145 | 1.00 | 25.78 | C |
| ATOM | 6066 | CD | GLN | B | 362 | −7.741 | −31.106 | 99.998 | 1.00 | 29.85 | C |
| ATOM | 6067 | OE1 | GLN | B | 362 | −8.091 | −32.286 | 99.915 | 1.00 | 33.74 | O |
| ATOM | 6068 | NE2 | GLN | B | 362 | −8.297 | −30.238 | 100.850 | 1.00 | 31.65 | N |
| ATOM | 6069 | C | GLN | B | 362 | −3.032 | −29.646 | 99.926 | 1.00 | 22.72 | C |
| ATOM | 6070 | O | GLN | B | 362 | −3.311 | −29.043 | 100.958 | 1.00 | 23.20 | O |
| ATOM | 6079 | N | LEU | B | 363 | −1.808 | −29.709 | 99.425 | 1.00 | 22.13 | N |
| ATOM | 6080 | CA | LEU | B | 363 | −0.698 | −28.974 | 100.025 | 1.00 | 21.58 | C |
| ATOM | 6081 | CB | LEU | B | 363 | 0.596 | −29.233 | 99.242 | 1.00 | 21.22 | C |
| ATOM | 6082 | CG | LEU | B | 363 | 1.847 | −28.485 | 99.721 | 1.00 | 21.10 | C |
| ATOM | 6083 | CD1 | LEU | B | 363 | 2.469 | −29.087 | 100.968 | 1.00 | 20.20 | C |
| ATOM | 6084 | CD2 | LEU | B | 363 | 2.837 | −28.462 | 98.603 | 1.00 | 21.96 | C |
| ATOM | 6085 | C | LEU | B | 363 | −0.977 | −27.463 | 100.095 | 1.00 | 21.21 | C |
| ATOM | 6086 | O | LEU | B | 363 | −1.315 | −26.848 | 99.088 | 1.00 | 20.77 | O |
| ATOM | 6098 | N | ASP | B | 364 | −0.816 | −26.883 | 101.284 | 1.00 | 21.16 | N |
| ATOM | 6099 | CA | ASP | B | 364 | −0.986 | −25.446 | 101.481 | 1.00 | 21.13 | C |
| ATOM | 6100 | CB | ASP | B | 364 | −2.158 | −25.136 | 102.438 | 1.00 | 21.52 | C |
| ATOM | 6101 | CG | ASP | B | 364 | −1.879 | −25.510 | 103.881 | 1.00 | 21.50 | C |
| ATOM | 6102 | OD1 | ASP | B | 364 | −0.725 | −25.818 | 104.233 | 1.00 | 20.48 | O |
| ATOM | 6103 | OD2 | ASP | B | 364 | −2.780 | −25.485 | 104.738 | 1.00 | 22.01 | O |
| ATOM | 6104 | C | ASP | B | 364 | 0.317 | −24.814 | 101.925 | 1.00 | 20.71 | C |
| ATOM | 6105 | O | ASP | B | 364 | 1.288 | −25.510 | 102.166 | 1.00 | 21.50 | O |
| ATOM | 6110 | N | ARG | B | 365 | 0.339 | −23.497 | 102.027 | 1.00 | 20.55 | N |
| ATOM | 6111 | CA | ARG | B | 365 | 1.590 | −22.754 | 102.219 | 1.00 | 20.47 | C |
| ATOM | 6112 | CB | ARG | B | 365 | 1.341 | −21.267 | 102.085 | 1.00 | 20.98 | C |
| ATOM | 6113 | CG | ARG | B | 365 | 2.615 | −20.434 | 101.922 | 1.00 | 22.30 | C |
| ATOM | 6114 | CD | ARG | B | 365 | 2.297 | −18.953 | 101.742 | 1.00 | 23.78 | C |
| ATOM | 6115 | NE | ARG | B | 365 | 3.450 | −18.068 | 101.575 | 1.00 | 24.72 | N |
| ATOM | 6116 | CZ | ARG | B | 365 | 4.252 | −18.042 | 100.508 | 1.00 | 26.85 | C |
| ATOM | 6117 | NH1 | ARG | B | 365 | 4.076 | −18.874 | 99.482 | 1.00 | 28.00 | N |
| ATOM | 6118 | NH2 | ARG | B | 365 | 5.250 | −17.164 | 100.456 | 1.00 | 27.80 | N |
| ATOM | 6119 | C | ARG | B | 365 | 2.265 | −23.029 | 103.548 | 1.00 | 20.21 | C |
| ATOM | 6120 | O | ARG | B | 365 | 3.485 | −22.979 | 103.629 | 1.00 | 19.68 | O |
| ATOM | 6134 | N | GLN | B | 366 | 1.453 | −23.315 | 104.567 | 1.00 | 20.52 | N |
| ATOM | 6135 | CA | GLN | B | 366 | 1.893 | −23.691 | 105.909 | 1.00 | 20.24 | C |
| ATOM | 6136 | CB | GLN | B | 366 | 0.675 | −23.868 | 106.819 | 1.00 | 20.43 | C |
| ATOM | 6137 | CG | GLN | B | 366 | −0.015 | −22.576 | 107.275 | 1.00 | 21.31 | C |
| ATOM | 6138 | CD | GLN | B | 366 | −0.887 | −21.892 | 106.208 | 1.00 | 21.89 | C |
| ATOM | 6139 | OE1 | GLN | B | 366 | −1.449 | −22.644 | 105.298 | 1.00 | 26.18 | O |
| ATOM | 6140 | NE2 | GLN | B | 366 | −1.047 | −20.692 | 106.247 | 1.00 | 20.22 | N |
| ATOM | 6141 | C | GLN | B | 366 | 2.693 | −25.003 | 105.891 | 1.00 | 20.06 | C |
| ATOM | 6142 | O | GLN | B | 366 | 3.761 | −25.113 | 106.501 | 1.00 | 19.35 | O |
| ATOM | 6151 | N | GLU | B | 367 | 2.157 | −25.995 | 105.190 | 1.00 | 20.36 | N |
| ATOM | 6152 | CA | GLU | B | 367 | 2.846 | −27.267 | 105.011 | 1.00 | 20.59 | C |
| ATOM | 6153 | CB | GLU | B | 367 | 1.892 | −28.308 | 104.410 | 1.00 | 20.23 | C |
| ATOM | 6154 | CG | GLU | B | 367 | 0.767 | −28.689 | 105.352 | 1.00 | 20.47 | C |
| ATOM | 6155 | CD | GLU | B | 367 | −0.540 | −28.989 | 104.658 | 1.00 | 19.18 | C |
| ATOM | 6156 | OE1 | GLU | B | 367 | −0.583 | −28.880 | 103.416 | 1.00 | 19.11 | O |
| ATOM | 6157 | OE2 | GLU | B | 367 | −1.520 | −29.329 | 105.363 | 1.00 | 18.42 | O |
| ATOM | 6158 | C | GLU | B | 367 | 4.097 | −27.097 | 104.137 | 1.00 | 20.64 | C |
| ATOM | 6159 | O | GLU | B | 367 | 5.151 | −27.637 | 104.455 | 1.00 | 22.04 | O |
| ATOM | 6166 | N | PHE | B | 368 | 3.978 | −26.333 | 103.058 | 1.00 | 20.10 | N |
| ATOM | 6167 | CA | PHE | B | 368 | 5.101 | −26.038 | 102.194 | 1.00 | 20.21 | C |
| ATOM | 6168 | CB | PHE | B | 368 | 4.706 | −25.047 | 101.090 | 1.00 | 20.31 | C |
| ATOM | 6169 | CG | PHE | B | 368 | 5.881 | −24.502 | 100.344 | 1.00 | 20.83 | C |
| ATOM | 6170 | CD1 | PHE | B | 368 | 6.695 | −25.351 | 99.600 | 1.00 | 21.73 | C |
| ATOM | 6171 | CE1 | PHE | B | 368 | 7.787 | −24.872 | 98.952 | 1.00 | 21.46 | C |
| ATOM | 6172 | CZ | PHE | B | 368 | 8.105 | −23.519 | 99.022 | 1.00 | 21.70 | C |
| ATOM | 6173 | CE2 | PHE | B | 368 | 7.311 | −22.657 | 99.744 | 1.00 | 21.28 | C |
| ATOM | 6174 | CD2 | PHE | B | 368 | 6.203 | −23.149 | 100.412 | 1.00 | 21.89 | C |
| ATOM | 6175 | C | PHE | B | 368 | 6.290 | −25.496 | 102.956 | 1.00 | 19.85 | C |
| ATOM | 6176 | O | PHE | B | 368 | 7.373 | −26.026 | 102.851 | 1.00 | 19.48 | O |
| ATOM | 6186 | N | VAL | B | 369 | 6.088 | −24.430 | 103.716 | 1.00 | 20.23 | N |
| ATOM | 6187 | CA | VAL | B | 369 | 7.199 | −23.783 | 104.389 | 1.00 | 20.13 | C |
| ATOM | 6188 | CB | VAL | B | 369 | 6.864 | −22.360 | 104.958 | 1.00 | 20.25 | C |
| ATOM | 6189 | CG1 | VAL | B | 369 | 6.477 | −21.385 | 103.842 | 1.00 | 20.69 | C |
| ATOM | 6190 | CG2 | VAL | B | 369 | 5.822 | −22.386 | 106.090 | 1.00 | 20.83 | C |
| ATOM | 6191 | C | VAL | B | 369 | 7.787 | −24.667 | 105.479 | 1.00 | 20.45 | C |
| ATOM | 6192 | O | VAL | B | 369 | 8.992 | −24.602 | 105.747 | 1.00 | 20.34 | O |
| ATOM | 6202 | N | CYS | B | 370 | 6.950 | −25.508 | 106.091 | 1.00 | 20.80 | N |
| ATOM | 6203 | CA | CYS | B | 370 | 7.420 | −26.397 | 107.153 | 1.00 | 20.77 | C |
| ATOM | 6204 | CB | CYS | B | 370 | 6.264 | −27.006 | 107.940 | 1.00 | 21.11 | C |
| ATOM | 6205 | SG | CYS | B | 370 | 6.773 | −27.757 | 109.500 | 1.00 | 20.59 | S |
| ATOM | 6206 | C | CYS | B | 370 | 8.271 | −27.497 | 106.578 | 1.00 | 20.20 | C |
| ATOM | 6207 | O | CYS | B | 370 | 9.309 | −27.814 | 107.125 | 1.00 | 19.56 | O |
| ATOM | 6213 | N | LEU | B | 371 | 7.803 | −28.056 | 105.469 | 1.00 | 20.92 | N |
| ATOM | 6214 | CA | LEU | B | 371 | 8.539 | −29.049 | 104.687 | 1.00 | 21.60 | C |
| ATOM | 6215 | CB | LEU | B | 371 | 7.677 | −29.557 | 103.540 | 1.00 | 21.77 | C |
| ATOM | 6216 | CG | LEU | B | 371 | 6.513 | −30.454 | 103.984 | 1.00 | 21.18 | C |
| ATOM | 6217 | CD1 | LEU | B | 371 | 5.506 | −30.539 | 102.876 | 1.00 | 22.84 | C |
| ATOM | 6218 | CD2 | LEU | B | 371 | 6.964 | −31.867 | 104.379 | 1.00 | 20.73 | C |
| ATOM | 6219 | C | LEU | B | 371 | 9.878 | −28.572 | 104.143 | 1.00 | 22.28 | C |
| ATOM | 6220 | O | LEU | B | 371 | 10.822 | −29.331 | 104.132 | 1.00 | 22.85 | O |
| ATOM | 6232 | N | LYS | B | 372 | 9.954 | −27.323 | 103.695 | 1.00 | 22.83 | N |
| ATOM | 6233 | CA | LYS | B | 372 | 11.227 | −26.698 | 103.307 | 1.00 | 23.30 | C |
| ATOM | 6234 | CB | LYS | B | 372 | 11.009 | −25.223 | 102.901 | 1.00 | 24.00 | C |
| ATOM | 6235 | CG | LYS | B | 372 | 11.086 | −24.886 | 101.456 | 1.00 | 24.89 | C |
| ATOM | 6236 | CD | LYS | B | 372 | 10.746 | −23.409 | 101.260 | 1.00 | 26.29 | C |
| ATOM | 6237 | CE | LYS | B | 372 | 11.815 | −22.520 | 101.826 | 1.00 | 28.79 | C |
| ATOM | 6238 | NZ | LYS | B | 372 | 12.282 | −21.355 | 100.973 | 1.00 | 32.44 | N |
| ATOM | 6239 | C | LYS | B | 372 | 12.222 | −26.703 | 104.475 | 1.00 | 22.50 | C |
| ATOM | 6240 | O | LYS | B | 372 | 13.410 | −26.928 | 104.283 | 1.00 | 21.79 | O |
| ATOM | 6254 | N | PHE | B | 373 | 11.720 | −26.411 | 105.675 | 1.00 | 21.75 | N |
| ATOM | 6255 | CA | PHE | B | 373 | 12.538 | −26.397 | 106.881 | 1.00 | 21.48 | C |
| ATOM | 6256 | CB | PHE | B | 373 | 11.752 | −25.711 | 108.006 | 1.00 | 21.59 | C |
| ATOM | 6257 | CG | PHE | B | 373 | 12.568 | −25.400 | 109.234 | 1.00 | 22.30 | C |
| ATOM | 6258 | CD1 | PHE | B | 373 | 13.254 | −24.182 | 109.352 | 1.00 | 23.47 | C |
| ATOM | 6259 | CE1 | PHE | B | 373 | 13.988 | −23.875 | 110.519 | 1.00 | 23.22 | C |
| ATOM | 6260 | CZ | PHE | B | 373 | 14.046 | −24.791 | 111.572 | 1.00 | 22.77 | C |
| ATOM | 6261 | CE2 | PHE | B | 373 | 13.364 | −26.010 | 111.453 | 1.00 | 22.66 | C |
| ATOM | 6262 | CD2 | PHE | B | 373 | 12.630 | −26.305 | 110.294 | 1.00 | 22.09 | C |
| ATOM | 6263 | C | PHE | B | 373 | 13.013 | −27.796 | 107.317 | 1.00 | 21.12 | C |
| ATOM | 6264 | O | PHE | B | 373 | 14.158 | −27.994 | 107.775 | 1.00 | 20.29 | O |
| ATOM | 6274 | N | ILE | B | 374 | 12.105 | −28.751 | 107.197 | 1.00 | 20.74 | N |
| ATOM | 6275 | CA | ILE | B | 374 | 12.425 | −30.145 | 107.402 | 1.00 | 20.61 | C |
| ATOM | 6276 | CB | ILE | B | 374 | 11.153 | −31.001 | 107.227 | 1.00 | 20.39 | C |
| ATOM | 6277 | CG1 | ILE | B | 374 | 10.213 | −30.798 | 108.438 | 1.00 | 20.30 | C |
| ATOM | 6278 | CD1 | ILE | B | 374 | 8.746 | −31.217 | 108.223 | 1.00 | 19.72 | C |
| ATOM | 6279 | CG2 | ILE | B | 374 | 11.512 | −32.485 | 107.049 | 1.00 | 20.70 | C |
| ATOM | 6280 | C | ILE | B | 374 | 13.545 | −30.619 | 106.469 | 1.00 | 20.48 | C |
| ATOM | 6281 | O | ILE | B | 374 | 14.441 | −31.322 | 106.904 | 1.00 | 20.58 | O |
| ATOM | 6293 | N | ILE | B | 375 | 13.476 | −30.251 | 105.200 | 1.00 | 20.37 | N |
| ATOM | 6294 | CA | ILE | B | 375 | 14.477 | −30.667 | 104.233 | 1.00 | 20.81 | C |
| ATOM | 6295 | CB | ILE | B | 375 | 14.116 | −30.171 | 102.795 | 1.00 | 20.54 | C |
| ATOM | 6296 | CG1 | ILE | B | 375 | 12.958 | −30.976 | 102.224 | 1.00 | 20.00 | C |
| ATOM | 6297 | CD1 | ILE | B | 375 | 12.338 | −30.334 | 100.985 | 1.00 | 19.63 | C |
| ATOM | 6298 | CG2 | ILE | B | 375 | 15.370 | −30.176 | 101.819 | 1.00 | 21.08 | C |
| ATOM | 6299 | C | ILE | B | 375 | 15.820 | −30.087 | 104.646 | 1.00 | 21.03 | C |
| ATOM | 6300 | O | ILE | B | 375 | 16.849 | −30.772 | 104.601 | 1.00 | 20.52 | O |
| ATOM | 6312 | N | LEU | B | 376 | 15.786 | −28.810 | 105.016 | 1.00 | 21.04 | N |
| ATOM | 6313 | CA | LEU | B | 376 | 16.960 | −28.085 | 105.434 | 1.00 | 21.32 | C |
| ATOM | 6314 | CB | LEU | B | 376 | 16.567 | −26.669 | 105.892 | 1.00 | 21.62 | C |
| ATOM | 6315 | CG | LEU | B | 376 | 17.668 | −25.769 | 106.479 | 1.00 | 22.29 | C |
| ATOM | 6316 | CD1 | LEU | B | 376 | 18.818 | −25.620 | 105.506 | 1.00 | 23.14 | C |
| ATOM | 6317 | CD2 | LEU | B | 376 | 17.127 | −24.389 | 106.897 | 1.00 | 22.61 | C |
| ATOM | 6318 | C | LEU | B | 376 | 17.704 | −28.863 | 106.536 | 1.00 | 21.60 | C |
| ATOM | 6319 | O | LEU | B | 376 | 18.919 | −29.071 | 106.442 | 1.00 | 20.65 | O |
| ATOM | 6331 | N | PHE | B | 377 | 16.966 | −29.334 | 107.544 | 1.00 | 21.48 | N |
| ATOM | 6332 | CA | PHE | B | 377 | 17.571 | −30.010 | 108.681 | 1.00 | 21.51 | C |
| ATOM | 6333 | CB | PHE | B | 377 | 16.918 | −29.529 | 109.980 | 1.00 | 21.37 | C |
| ATOM | 6334 | CG | PHE | B | 377 | 17.438 | −28.198 | 110.463 | 1.00 | 21.13 | C |
| ATOM | 6335 | CD1 | PHE | B | 377 | 16.814 | −27.012 | 110.107 | 1.00 | 20.61 | C |
| ATOM | 6336 | CE1 | PHE | B | 377 | 17.299 | −25.775 | 110.576 | 1.00 | 20.67 | C |
| ATOM | 6337 | CZ | PHE | B | 377 | 18.409 | −25.734 | 111.378 | 1.00 | 20.49 | C |
| ATOM | 6338 | CE2 | PHE | B | 377 | 19.042 | −26.908 | 111.745 | 1.00 | 21.35 | C |
| ATOM | 6339 | CD2 | PHE | B | 377 | 18.559 | −28.130 | 111.279 | 1.00 | 21.81 | C |
| ATOM | 6340 | C | PHE | B | 377 | 17.535 | −31.538 | 108.584 | 1.00 | 22.05 | C |
| ATOM | 6341 | O | PHE | B | 377 | 17.823 | −32.214 | 109.543 | 1.00 | 22.25 | O |
| ATOM | 6351 | N | SER | B | 378 | 17.225 | −32.083 | 107.413 | 1.00 | 23.24 | N |
| ATOM | 6352 | CA | SER | B | 378 | 17.149 | −33.532 | 107.212 | 1.00 | 23.97 | C |
| ATOM | 6353 | CB | SER | B | 378 | 16.253 | −33.854 | 106.031 | 1.00 | 23.80 | C |
| ATOM | 6354 | OG | SER | B | 378 | 16.701 | −33.159 | 104.859 | 1.00 | 26.53 | O |
| ATOM | 6355 | C | SER | B | 378 | 18.523 | −34.065 | 106.921 | 1.00 | 24.41 | C |
| ATOM | 6356 | O | SER | B | 378 | 18.851 | −34.325 | 105.776 | 1.00 | 25.79 | O |
| ATOM | 6362 | N | LEU | B | 379 | 19.349 | −34.164 | 107.944 | 1.00 | 24.72 | N |
| ATOM | 6363 | CA | LEU | B | 379 | 20.692 | −34.706 | 107.803 | 1.00 | 24.56 | C |
| ATOM | 6364 | CB | LEU | B | 379 | 21.736 | −33.611 | 107.606 | 1.00 | 24.22 | C |
| ATOM | 6365 | CG | LEU | B | 379 | 23.177 | −34.096 | 107.371 | 1.00 | 22.91 | C |
| ATOM | 6366 | CD1 | LEU | B | 379 | 23.307 | −35.123 | 106.239 | 1.00 | 22.01 | C |
| ATOM | 6367 | CD2 | LEU | B | 379 | 24.080 | −32.919 | 107.097 | 1.00 | 22.89 | C |
| ATOM | 6368 | C | LEU | B | 379 | 20.998 | −35.454 | 109.063 | 1.00 | 25.35 | C |
| ATOM | 6369 | O | LEU | B | 379 | 20.855 | −34.913 | 110.160 | 1.00 | 25.39 | O |
| ATOM | 6381 | N | ASP | B | 380 | 21.427 | −36.702 | 108.916 | 1.00 | 26.30 | N |
| ATOM | 6382 | CA | ASP | B | 380 | 21.766 | −37.495 | 110.080 | 1.00 | 26.69 | C |
| ATOM | 6383 | CB | ASP | B | 380 | 22.405 | −38.832 | 109.705 | 1.00 | 26.94 | C |
| ATOM | 6384 | CG | ASP | B | 380 | 22.343 | −39.827 | 110.839 | 1.00 | 28.24 | C |
| ATOM | 6385 | OD1 | ASP | B | 380 | 21.298 | −39.835 | 111.542 | 1.00 | 30.15 | O |
| ATOM | 6386 | OD2 | ASP | B | 380 | 23.277 | −40.623 | 111.118 | 1.00 | 29.59 | O |
| ATOM | 6387 | C | ASP | B | 380 | 22.712 | −36.705 | 110.954 | 1.00 | 26.83 | C |
| ATOM | 6388 | O | ASP | B | 380 | 23.650 | −36.068 | 110.455 | 1.00 | 26.94 | O |
| ATOM | 6393 | N | LEU | B | 381 | 22.448 | −36.772 | 112.259 | 1.00 | 26.95 | N |
| ATOM | 6394 | CA | LEU | B | 381 | 23.122 | −35.958 | 113.272 | 1.00 | 26.72 | C |
| ATOM | 6395 | CB | LEU | B | 381 | 22.480 | −36.172 | 114.648 | 1.00 | 26.90 | C |
| ATOM | 6396 | CG | LEU | B | 381 | 21.569 | −35.075 | 115.200 | 1.00 | 26.71 | C |
| ATOM | 6397 | CD1 | LEU | B | 381 | 20.429 | −34.799 | 114.274 | 1.00 | 26.78 | C |
| ATOM | 6398 | CD2 | LEU | B | 381 | 21.046 | −35.500 | 116.557 | 1.00 | 26.73 | C |
| ATOM | 6399 | C | LEU | B | 381 | 24.578 | −36.325 | 113.369 | 1.00 | 26.82 | C |
| ATOM | 6400 | O | LEU | B | 381 | 25.409 | −35.482 | 113.693 | 1.00 | 26.46 | O |
| ATOM | 6412 | N | LYS | B | 382 | 24.856 | −37.600 | 113.095 | 1.00 | 27.09 | N |
| ATOM | 6413 | CA | LYS | B | 382 | 26.202 | −38.181 | 113.120 | 1.00 | 27.16 | C |
| ATOM | 6414 | CB | LYS | B | 382 | 26.148 | −39.644 | 112.593 | 1.00 | 27.31 | C |
| ATOM | 6415 | CG | LYS | B | 382 | 27.392 | −40.181 | 111.841 | 1.00 | 27.83 | C |
| ATOM | 6416 | CD | LYS | B | 382 | 27.058 | −41.351 | 110.886 | 1.00 | 28.33 | C |
| ATOM | 6417 | CE | LYS | B | 382 | 27.868 | −41.262 | 109.580 | 1.00 | 28.67 | C |
| ATOM | 6418 | NZ | LYS | B | 382 | 27.961 | −42.547 | 108.823 | 1.00 | 28.78 | N |
| ATOM | 6419 | C | LYS | B | 382 | 27.274 | −37.343 | 112.395 | 1.00 | 27.03 | C |
| ATOM | 6420 | O | LYS | B | 382 | 28.462 | −37.489 | 112.704 | 1.00 | 27.15 | O |
| ATOM | 6434 | N | PHE | B | 383 | 26.890 | −36.473 | 111.453 | 1.00 | 26.97 | N |
| ATOM | 6435 | CA | PHE | B | 383 | 27.896 | −35.577 | 110.852 | 1.00 | 27.01 | C |
| ATOM | 6436 | CB | PHE | B | 383 | 28.407 | −36.026 | 109.448 | 1.00 | 27.55 | C |
| ATOM | 6437 | CG | PHE | B | 383 | 27.382 | −36.673 | 108.549 | 1.00 | 28.72 | C |
| ATOM | 6438 | CD1 | PHE | B | 383 | 27.171 | −36.165 | 107.274 | 1.00 | 31.72 | C |
| ATOM | 6439 | CE1 | PHE | B | 383 | 26.254 | −36.770 | 106.405 | 1.00 | 33.05 | C |
| ATOM | 6440 | CZ | PHE | B | 383 | 25.570 | −37.921 | 106.823 | 1.00 | 32.42 | C |
| ATOM | 6441 | CE2 | PHE | B | 383 | 25.807 | −38.445 | 108.081 | 1.00 | 30.18 | C |
| ATOM | 6442 | CD2 | PHE | B | 383 | 26.715 | −37.833 | 108.924 | 1.00 | 29.87 | C |
| ATOM | 6443 | C | PHE | B | 383 | 27.628 | −34.066 | 110.909 | 1.00 | 25.92 | C |
| ATOM | 6444 | O | PHE | B | 383 | 27.923 | −33.341 | 109.971 | 1.00 | 25.81 | O |
| ATOM | 6454 | N | LEU | B | 384 | 27.142 | −33.605 | 112.053 | 1.00 | 25.16 | N |
| ATOM | 6455 | CA | LEU | B | 384 | 27.258 | −32.203 | 112.432 | 1.00 | 24.71 | C |
| ATOM | 6456 | CB | LEU | B | 384 | 25.883 | −31.556 | 112.525 | 1.00 | 24.63 | C |
| ATOM | 6457 | CG | LEU | B | 384 | 25.175 | −31.470 | 111.167 | 1.00 | 24.97 | C |
| ATOM | 6458 | CD1 | LEU | B | 384 | 23.750 | −31.977 | 111.293 | 1.00 | 25.36 | C |
| ATOM | 6459 | CD2 | LEU | B | 384 | 25.221 | −30.069 | 110.545 | 1.00 | 24.62 | C |
| ATOM | 6460 | C | LEU | B | 384 | 27.989 | −32.078 | 113.761 | 1.00 | 24.36 | C |
| ATOM | 6461 | O | LEU | B | 384 | 28.133 | −33.037 | 114.506 | 1.00 | 24.77 | O |
| ATOM | 6473 | N | ASN | B | 385 | 28.475 | −30.885 | 114.045 | 1.00 | 23.95 | N |
| ATOM | 6474 | CA | ASN | B | 385 | 29.093 | −30.605 | 115.316 | 1.00 | 23.43 | C |
| ATOM | 6475 | CB | ASN | B | 385 | 29.884 | −29.303 | 115.241 | 1.00 | 23.49 | C |
| ATOM | 6476 | CG | ASN | B | 385 | 30.973 | −29.334 | 114.180 | 1.00 | 24.09 | C |
| ATOM | 6477 | OD1 | ASN | B | 385 | 31.633 | −30.484 | 114.029 | 1.00 | 24.69 | O |
| ATOM | 6478 | ND2 | ASN | B | 385 | 31.221 | −28.333 | 113.506 | 1.00 | 24.86 | N |
| ATOM | 6479 | C | ASN | B | 385 | 28.022 | −30.496 | 116.375 | 1.00 | 23.20 | C |
| ATOM | 6480 | O | ASN | B | 385 | 28.058 | −31.206 | 117.374 | 1.00 | 23.23 | O |
| ATOM | 6487 | N | ASN | B | 386 | 27.062 | −29.608 | 116.136 | 1.00 | 23.03 | N |
| ATOM | 6488 | CA | ASN | B | 386 | 26.027 | −29.285 | 117.108 | 1.00 | 23.15 | C |
| ATOM | 6489 | CB | ASN | B | 386 | 25.682 | −27.788 | 117.053 | 1.00 | 23.30 | C |
| ATOM | 6490 | CG | ASN | B | 386 | 24.910 | −27.309 | 118.284 | 1.00 | 23.81 | C |
| ATOM | 6491 | OD1 | ASN | B | 386 | 24.112 | −28.039 | 118.844 | 1.00 | 24.01 | O |
| ATOM | 6492 | ND2 | ASN | B | 386 | 25.166 | −26.075 | 118.711 | 1.00 | 25.49 | N |
| ATOM | 6493 | C | ASN | B | 386 | 24.778 | −30.117 | 116.905 | 1.00 | 23.00 | C |
| ATOM | 6494 | O | ASN | B | 386 | 23.776 | −29.634 | 116.404 | 1.00 | 23.29 | O |
| ATOM | 6501 | N | HIS | B | 387 | 24.852 | −31.370 | 117.335 | 1.00 | 23.13 | N |
| ATOM | 6502 | CA | HIS | B | 387 | 23.706 | −32.273 | 117.380 | 1.00 | 23.03 | C |
| ATOM | 6503 | CB | HIS | B | 387 | 24.101 | −33.593 | 118.061 | 1.00 | 23.23 | C |
| ATOM | 6504 | CG | HIS | B | 387 | 25.414 | −34.141 | 117.607 | 1.00 | 23.26 | C |
| ATOM | 6505 | ND1 | HIS | B | 387 | 25.677 | −34.451 | 116.291 | 1.00 | 24.07 | N |
| ATOM | 6506 | CE1 | HIS | B | 387 | 26.913 | −34.906 | 116.186 | 1.00 | 23.05 | C |
| ATOM | 6507 | NE2 | HIS | B | 387 | 27.468 | −34.876 | 117.380 | 1.00 | 23.67 | N |
| ATOM | 6508 | CD2 | HIS | B | 387 | 26.551 | −34.403 | 118.289 | 1.00 | 24.20 | C |
| ATOM | 6509 | C | HIS | B | 387 | 22.518 | −31.690 | 118.145 | 1.00 | 22.88 | C |
| ATOM | 6510 | O | HIS | B | 387 | 21.380 | −32.006 | 117.847 | 1.00 | 23.07 | O |
| ATOM | 6519 | N | ILE | B | 388 | 22.775 | −30.863 | 119.148 | 1.00 | 22.86 | N |
| ATOM | 6520 | CA | ILE | B | 388 | 21.705 | −30.440 | 120.047 | 1.00 | 23.14 | C |
| ATOM | 6521 | CB | ILE | B | 388 | 22.279 | −29.988 | 121.461 | 1.00 | 23.40 | C |
| ATOM | 6522 | CG1 | ILE | B | 388 | 21.158 | −29.728 | 122.464 | 1.00 | 23.09 | C |
| ATOM | 6523 | CD1 | ILE | B | 388 | 21.612 | −29.788 | 123.872 | 1.00 | 23.90 | C |
| ATOM | 6524 | CG2 | ILE | B | 388 | 23.186 | −28.765 | 121.379 | 1.00 | 24.25 | C |
| ATOM | 6525 | C | ILE | B | 388 | 20.742 | −29.434 | 119.385 | 1.00 | 23.07 | C |
| ATOM | 6526 | O | ILE | B | 388 | 19.525 | −29.619 | 119.424 | 1.00 | 22.78 | O |
| ATOM | 6538 | N | LEU | B | 389 | 21.278 | −28.415 | 118.730 | 1.00 | 23.05 | N |
| ATOM | 6539 | CA | LEU | B | 389 | 20.436 | −27.420 | 118.063 | 1.00 | 23.18 | C |
| ATOM | 6540 | CB | LEU | B | 389 | 21.264 | −26.242 | 117.534 | 1.00 | 23.08 | C |
| ATOM | 6541 | CG | LEU | B | 389 | 21.149 | −24.909 | 118.281 | 1.00 | 23.42 | C |
| ATOM | 6542 | CD1 | LEU | B | 389 | 21.217 | −25.086 | 119.795 | 1.00 | 23.09 | C |
| ATOM | 6543 | CD2 | LEU | B | 389 | 22.246 | −23.946 | 117.801 | 1.00 | 24.17 | C |
| ATOM | 6544 | C | LEU | B | 389 | 19.696 | −28.052 | 116.911 | 1.00 | 23.20 | C |
| ATOM | 6545 | O | LEU | B | 389 | 18.552 | −27.715 | 116.643 | 1.00 | 23.38 | O |
| ATOM | 6557 | N | VAL | B | 390 | 20.374 | −28.964 | 116.228 | 1.00 | 23.24 | N |
| ATOM | 6558 | CA | VAL | B | 390 | 19.841 | −29.618 | 115.046 | 1.00 | 23.18 | C |
| ATOM | 6559 | CB | VAL | B | 390 | 20.968 | −30.338 | 114.302 | 1.00 | 23.10 | C |
| ATOM | 6560 | CG1 | VAL | B | 390 | 20.417 | −31.332 | 113.284 | 1.00 | 23.45 | C |
| ATOM | 6561 | CG2 | VAL | B | 390 | 21.887 | −29.292 | 113.639 | 1.00 | 23.10 | C |
| ATOM | 6562 | C | VAL | B | 390 | 18.733 | −30.595 | 115.395 | 1.00 | 23.39 | C |
| ATOM | 6563 | O | VAL | B | 390 | 17.710 | −30.615 | 114.744 | 1.00 | 23.16 | O |
| ATOM | 6573 | N | LYS | B | 391 | 18.958 | −31.404 | 116.422 | 1.00 | 24.08 | N |
| ATOM | 6574 | CA | LYS | B | 391 | 17.966 | −32.347 | 116.918 | 1.00 | 24.62 | C |
| ATOM | 6575 | CB | LYS | B | 391 | 18.548 | −33.125 | 118.087 | 1.00 | 24.73 | C |
| ATOM | 6576 | CG | LYS | B | 391 | 17.776 | −34.377 | 118.453 | 1.00 | 25.84 | C |
| ATOM | 6577 | CD | LYS | B | 391 | 18.317 | −34.982 | 119.760 | 1.00 | 26.49 | C |
| ATOM | 6578 | CE | LYS | B | 391 | 17.519 | −34.555 | 121.015 | 1.00 | 26.94 | C |
| ATOM | 6579 | NZ | LYS | B | 391 | 17.125 | −35.746 | 121.827 | 1.00 | 26.96 | N |
| ATOM | 6580 | C | LYS | B | 391 | 16.687 | −31.625 | 117.346 | 1.00 | 24.82 | C |
| ATOM | 6581 | O | LYS | B | 391 | 15.599 | −32.005 | 116.930 | 1.00 | 24.88 | O |
| ATOM | 6595 | N | ASP | B | 392 | 16.836 | −30.588 | 118.175 | 1.00 | 25.21 | N |
| ATOM | 6596 | CA | ASP | B | 392 | 15.739 | −29.671 | 118.548 | 1.00 | 25.44 | C |
| ATOM | 6597 | CB | ASP | B | 392 | 16.291 | −28.502 | 119.394 | 1.00 | 25.44 | C |
| ATOM | 6598 | CG | ASP | B | 392 | 15.306 | −27.327 | 119.528 | 1.00 | 25.66 | C |
| ATOM | 6599 | OD1 | ASP | B | 392 | 14.331 | −27.446 | 120.301 | 1.00 | 27.05 | O |
| ATOM | 6600 | OD2 | ASP | B | 392 | 15.435 | −26.240 | 118.917 | 1.00 | 25.54 | O |
| ATOM | 6601 | C | ASP | B | 392 | 14.979 | −29.139 | 117.315 | 1.00 | 25.52 | C |
| ATOM | 6602 | O | ASP | B | 392 | 13.748 | −29.162 | 117.286 | 1.00 | 26.32 | O |
| ATOM | 6607 | N | ALA | B | 393 | 15.721 | −28.677 | 116.305 | 1.00 | 25.09 | N |
| ATOM | 6608 | CA | ALA | B | 393 | 15.147 | −28.181 | 115.061 | 1.00 | 24.72 | C |
| ATOM | 6609 | CB | ALA | B | 393 | 16.229 | −27.571 | 114.188 | 1.00 | 24.90 | C |
| ATOM | 6610 | C | ALA | B | 393 | 14.403 | −29.263 | 114.280 | 1.00 | 24.18 | C |
| ATOM | 6611 | O | ALA | B | 393 | 13.329 | −29.003 | 113.752 | 1.00 | 24.12 | O |
| ATOM | 6617 | N | GLN | B | 394 | 14.995 | −30.456 | 114.201 | 1.00 | 23.48 | N |
| ATOM | 6618 | CA | GLN | B | 394 | 14.409 | −31.598 | 113.497 | 1.00 | 23.18 | C |
| ATOM | 6619 | CB | GLN | B | 394 | 15.394 | −32.791 | 113.483 | 1.00 | 22.91 | C |
| ATOM | 6620 | CG | GLN | B | 394 | 16.508 | −32.706 | 112.433 | 1.00 | 22.04 | C |
| ATOM | 6621 | CD | GLN | B | 394 | 17.537 | −33.848 | 112.487 | 1.00 | 21.23 | C |
| ATOM | 6622 | OE1 | GLN | B | 394 | 17.528 | −34.668 | 113.397 | 1.00 | 21.48 | O |
| ATOM | 6623 | NE2 | GLN | B | 394 | 18.424 | −33.887 | 111.501 | 1.00 | 20.15 | N |
| ATOM | 6624 | C | GLN | B | 394 | 13.095 | −32.017 | 114.176 | 1.00 | 23.38 | C |
| ATOM | 6625 | O | GLN | B | 394 | 12.052 | −32.172 | 113.531 | 1.00 | 23.34 | O |
| ATOM | 6634 | N | GLU | B | 395 | 13.168 | −32.166 | 115.493 | 1.00 | 23.47 | N |
| ATOM | 6635 | CA | GLU | B | 395 | 12.030 | −32.546 | 116.316 | 1.00 | 23.62 | C |
| ATOM | 6636 | CB | GLU | B | 395 | 12.510 | −32.778 | 117.762 | 1.00 | 23.68 | C |
| ATOM | 6637 | CG | GLU | B | 395 | 13.409 | −34.001 | 117.884 | 1.00 | 24.92 | C |
| ATOM | 6638 | CD | GLU | B | 395 | 14.178 | −34.109 | 119.203 | 1.00 | 27.89 | C |
| ATOM | 6639 | OE1 | GLU | B | 395 | 14.335 | −33.091 | 119.941 | 1.00 | 29.21 | O |
| ATOM | 6640 | OE2 | GLU | B | 395 | 14.642 | −35.241 | 119.493 | 1.00 | 27.49 | O |
| ATOM | 6641 | C | GLU | B | 395 | 10.894 | −31.511 | 116.280 | 1.00 | 23.16 | C |
| ATOM | 6642 | O | GLU | B | 395 | 9.734 | −31.868 | 116.154 | 1.00 | 22.93 | O |
| ATOM | 6649 | N | LYS | B | 396 | 11.238 | −30.237 | 116.393 | 1.00 | 23.08 | N |
| ATOM | 6650 | CA | LYS | B | 396 | 10.244 | −29.170 | 116.355 | 1.00 | 23.50 | C |
| ATOM | 6651 | CB | LYS | B | 396 | 10.914 | −27.816 | 116.625 | 1.00 | 23.66 | C |
| ATOM | 6652 | CG | LYS | B | 396 | 11.014 | −27.457 | 118.083 | 1.00 | 24.10 | C |
| ATOM | 6653 | CD | LYS | B | 396 | 12.056 | −26.372 | 118.335 | 1.00 | 25.74 | C |
| ATOM | 6654 | CE | LYS | B | 396 | 11.478 | −24.958 | 118.345 | 1.00 | 26.64 | C |
| ATOM | 6655 | NZ | LYS | B | 396 | 12.356 | −24.037 | 119.166 | 1.00 | 26.34 | N |
| ATOM | 6656 | C | LYS | B | 396 | 9.459 | −29.088 | 115.021 | 1.00 | 23.19 | C |
| ATOM | 6657 | O | LYS | B | 396 | 8.248 | −28.881 | 115.023 | 1.00 | 23.10 | O |
| ATOM | 6671 | N | ALA | B | 397 | 10.168 | −29.223 | 113.897 | 1.00 | 23.00 | N |
| ATOM | 6672 | CA | ALA | B | 397 | 9.568 | −29.128 | 112.561 | 1.00 | 22.47 | C |
| ATOM | 6673 | CB | ALA | B | 397 | 10.649 | −28.963 | 111.498 | 1.00 | 22.10 | C |
| ATOM | 6674 | C | ALA | B | 397 | 8.689 | −30.336 | 112.245 | 1.00 | 22.41 | C |
| ATOM | 6675 | O | ALA | B | 397 | 7.653 | −30.208 | 111.617 | 1.00 | 21.88 | O |
| ATOM | 6681 | N | ASN | B | 398 | 9.127 | −31.511 | 112.670 | 1.00 | 22.66 | N |
| ATOM | 6682 | CA | ASN | B | 398 | 8.338 | −32.718 | 112.539 | 1.00 | 23.18 | C |
| ATOM | 6683 | CB | ASN | B | 398 | 9.231 | −33.904 | 112.890 | 1.00 | 23.88 | C |
| ATOM | 6684 | CG | ASN | B | 398 | 8.564 | −35.231 | 112.689 | 1.00 | 26.26 | C |
| ATOM | 6685 | OD1 | ASN | B | 398 | 7.609 | −35.553 | 113.559 | 1.00 | 31.29 | O |
| ATOM | 6686 | ND2 | ASN | B | 398 | 8.922 | −35.987 | 111.780 | 1.00 | 29.41 | N |
| ATOM | 6687 | C | ASN | B | 398 | 7.088 | −32.636 | 113.446 | 1.00 | 22.52 | C |
| ATOM | 6688 | O | ASN | B | 398 | 5.996 | −33.031 | 113.045 | 1.00 | 22.40 | O |
| ATOM | 6695 | N | ALA | B | 399 | 7.243 | −32.079 | 114.646 | 1.00 | 22.04 | N |
| ATOM | 6696 | CA | ALA | B | 399 | 6.117 | −31.903 | 115.561 | 1.00 | 21.71 | C |
| ATOM | 6697 | CB | ALA | B | 399 | 6.609 | −31.600 | 116.967 | 1.00 | 21.86 | C |
| ATOM | 6698 | C | ALA | B | 399 | 5.153 | −30.814 | 115.097 | 1.00 | 21.68 | C |
| ATOM | 6699 | O | ALA | B | 399 | 3.955 | −30.965 | 115.230 | 1.00 | 21.61 | O |
| ATOM | 6705 | N | ALA | B | 400 | 5.676 | −29.720 | 114.551 | 1.00 | 21.65 | N |
| ATOM | 6706 | CA | ALA | B | 400 | 4.846 | −28.622 | 114.034 | 1.00 | 21.60 | C |
| ATOM | 6707 | CB | ALA | B | 400 | 5.722 | −27.425 | 113.605 | 1.00 | 22.00 | C |
| ATOM | 6708 | C | ALA | B | 400 | 3.971 | −29.065 | 112.872 | 1.00 | 21.44 | C |
| ATOM | 6709 | O | ALA | B | 400 | 2.784 | −28.735 | 112.808 | 1.00 | 20.65 | O |
| ATOM | 6715 | N | LEU | B | 401 | 4.568 | −29.812 | 111.952 | 1.00 | 21.36 | N |
| ATOM | 6716 | CA | LEU | B | 401 | 3.838 | −30.338 | 110.804 | 1.00 | 21.80 | C |
| ATOM | 6717 | CB | LEU | B | 401 | 4.789 | −30.966 | 109.780 | 1.00 | 21.64 | C |
| ATOM | 6718 | CG | LEU | B | 401 | 4.116 | −31.348 | 108.460 | 1.00 | 22.07 | C |
| ATOM | 6719 | CD1 | LEU | B | 401 | 3.762 | −30.114 | 107.614 | 1.00 | 22.71 | C |
| ATOM | 6720 | CD2 | LEU | B | 401 | 4.985 | −32.325 | 107.679 | 1.00 | 22.26 | C |
| ATOM | 6721 | C | LEU | B | 401 | 2.786 | −31.353 | 111.231 | 1.00 | 21.48 | C |
| ATOM | 6722 | O | LEU | B | 401 | 1.656 | −31.295 | 110.769 | 1.00 | 21.87 | O |
| ATOM | 6734 | N | LEU | B | 402 | 3.139 | −32.271 | 112.117 | 1.00 | 21.42 | N |
| ATOM | 6735 | CA | LEU | B | 402 | 2.172 | −33.271 | 112.527 | 1.00 | 21.58 | C |
| ATOM | 6736 | CB | LEU | B | 402 | 2.776 | −34.299 | 113.512 | 1.00 | 21.77 | C |
| ATOM | 6737 | CG | LEU | B | 402 | 1.825 | −35.348 | 114.146 | 1.00 | 21.18 | C |
| ATOM | 6738 | CD1 | LEU | B | 402 | 1.328 | −36.362 | 113.143 | 1.00 | 21.50 | C |
| ATOM | 6739 | CD2 | LEU | B | 402 | 2.484 | −36.064 | 115.270 | 1.00 | 19.93 | C |
| ATOM | 6740 | C | LEU | B | 402 | 0.973 | −32.557 | 113.132 | 1.00 | 21.32 | C |
| ATOM | 6741 | O | LEU | B | 402 | −0.149 | −32.804 | 112.735 | 1.00 | 21.16 | O |
| ATOM | 6753 | N | ASP | B | 403 | 1.232 | −31.663 | 114.079 | 1.00 | 21.07 | N |
| ATOM | 6754 | CA | ASP | B | 403 | 0.181 | −30.930 | 114.763 | 1.00 | 21.02 | C |
| ATOM | 6755 | CB | ASP | B | 403 | 0.774 | −29.940 | 115.763 | 1.00 | 21.36 | C |
| ATOM | 6756 | CG | ASP | B | 403 | −0.294 | −29.222 | 116.564 | 1.00 | 22.91 | C |
| ATOM | 6757 | OD1 | ASP | B | 403 | −0.742 | −28.135 | 116.137 | 1.00 | 25.78 | O |
| ATOM | 6758 | OD2 | ASP | B | 403 | −0.765 | −29.670 | 117.630 | 1.00 | 25.66 | O |
| ATOM | 6759 | C | ASP | B | 403 | −0.686 | −30.175 | 113.772 | 1.00 | 20.33 | C |
| ATOM | 6760 | O | ASP | B | 403 | −1.914 | −30.243 | 113.806 | 1.00 | 20.42 | O |
| ATOM | 6765 | N | TYR | B | 404 | −0.024 | −29.465 | 112.884 | 1.00 | 19.56 | N |
| ATOM | 6766 | CA | TYR | B | 404 | −0.702 | −28.596 | 111.963 | 1.00 | 19.32 | C |
| ATOM | 6767 | CB | TYR | B | 404 | 0.300 | −27.758 | 111.114 | 1.00 | 18.97 | C |
| ATOM | 6768 | CG | TYR | B | 404 | −0.423 | −26.955 | 110.067 | 1.00 | 17.95 | C |
| ATOM | 6769 | CD1 | TYR | B | 404 | −1.013 | −25.737 | 110.388 | 1.00 | 16.65 | C |
| ATOM | 6770 | CE1 | TYR | B | 404 | −1.738 | −25.019 | 109.425 | 1.00 | 16.18 | C |
| ATOM | 6771 | CZ | TYR | B | 404 | −1.881 | −25.534 | 108.148 | 1.00 | 15.09 | C |
| ATOM | 6772 | OH | TYR | B | 404 | −2.589 | −24.821 | 107.207 | 1.00 | 14.54 | O |
| ATOM | 6773 | CE2 | TYR | B | 404 | −1.314 | −26.751 | 107.827 | 1.00 | 15.16 | C |
| ATOM | 6774 | CD2 | TYR | B | 404 | −0.602 | −27.452 | 108.777 | 1.00 | 14.95 | C |
| ATOM | 6775 | C | TYR | B | 404 | −1.645 | −29.424 | 111.091 | 1.00 | 19.08 | C |
| ATOM | 6776 | O | TYR | B | 404 | −2.800 | −29.079 | 110.940 | 1.00 | 18.67 | O |
| ATOM | 6786 | N | THR | B | 405 | −1.153 | −30.526 | 110.540 | 1.00 | 19.41 | N |
| ATOM | 6787 | CA | THR | B | 405 | −1.936 | −31.303 | 109.577 | 1.00 | 19.66 | C |
| ATOM | 6788 | CB | THR | B | 405 | −1.078 | −32.365 | 108.829 | 1.00 | 19.37 | C |
| ATOM | 6789 | OG1 | THR | B | 405 | −0.309 | −33.155 | 109.748 | 1.00 | 20.23 | O |
| ATOM | 6790 | CG2 | THR | B | 405 | −0.039 | −31.699 | 107.991 | 1.00 | 19.49 | C |
| ATOM | 6791 | C | THR | B | 405 | −3.166 | −31.947 | 110.217 | 1.00 | 19.90 | C |
| ATOM | 6792 | O | THR | B | 405 | −4.242 | −31.978 | 109.625 | 1.00 | 19.53 | O |
| ATOM | 6800 | N | LEU | B | 406 | −3.038 | −32.454 | 111.429 | 1.00 | 20.50 | N |
| ATOM | 6801 | CA | LEU | B | 406 | −4.206 | −33.104 | 112.015 | 1.00 | 21.01 | C |
| ATOM | 6802 | CB | LEU | B | 406 | −3.840 | −34.319 | 112.878 | 1.00 | 21.44 | C |
| ATOM | 6803 | CG | LEU | B | 406 | −3.152 | −34.324 | 114.237 | 1.00 | 21.95 | C |
| ATOM | 6804 | CD1 | LEU | B | 406 | −2.159 | −35.471 | 114.248 | 1.00 | 22.14 | C |
| ATOM | 6805 | CD2 | LEU | B | 406 | −2.497 | −33.048 | 114.606 | 1.00 | 23.36 | C |
| ATOM | 6806 | C | LEU | B | 406 | −5.172 | −32.158 | 112.674 | 1.00 | 20.62 | C |
| ATOM | 6807 | O | LEU | B | 406 | −6.318 | −32.505 | 112.835 | 1.00 | 20.88 | O |
| ATOM | 6819 | N | CYS | B | 407 | −4.734 | −30.939 | 112.983 | 1.00 | 21.02 | N |
| ATOM | 6820 | CA | CYS | B | 407 | −5.629 | −29.918 | 113.533 | 1.00 | 21.06 | C |
| ATOM | 6821 | CB | CYS | B | 407 | −4.862 | −28.866 | 114.346 | 1.00 | 21.54 | C |
| ATOM | 6822 | SG | CYS | B | 407 | −4.329 | −29.426 | 116.000 | 1.00 | 25.17 | S |
| ATOM | 6823 | C | CYS | B | 407 | −6.384 | −29.216 | 112.435 | 1.00 | 20.23 | C |
| ATOM | 6824 | O | CYS | B | 407 | −7.530 | −28.847 | 112.628 | 1.00 | 19.72 | O |
| ATOM | 6830 | N | HIS | B | 408 | −5.728 | −29.015 | 111.292 | 1.00 | 20.11 | N |
| ATOM | 6831 | CA | HIS | B | 408 | −6.307 | −28.271 | 110.162 | 1.00 | 19.76 | C |
| ATOM | 6832 | CB | HIS | B | 408 | −5.253 | −27.361 | 109.528 | 1.00 | 19.87 | C |
| ATOM | 6833 | CG | HIS | B | 408 | −5.058 | −26.078 | 110.258 | 1.00 | 20.31 | C |
| ATOM | 6834 | ND1 | HIS | B | 408 | −5.567 | −24.884 | 109.803 | 1.00 | 22.45 | N |
| ATOM | 6835 | CE1 | HIS | B | 408 | −5.252 | −23.921 | 110.652 | 1.00 | 23.12 | C |
| ATOM | 6836 | NE2 | HIS | B | 408 | −4.560 | −24.450 | 111.646 | 1.00 | 22.32 | N |
| ATOM | 6837 | CD2 | HIS | B | 408 | −4.423 | −25.799 | 111.420 | 1.00 | 22.36 | C |
| ATOM | 6838 | C | HIS | B | 408 | −6.928 | −29.168 | 109.084 | 1.00 | 19.20 | C |
| ATOM | 6839 | O | HIS | B | 408 | −7.720 | −28.697 | 108.258 | 1.00 | 18.68 | O |
| ATOM | 6848 | N | TYR | B | 409 | −6.554 | −30.444 | 109.085 | 1.00 | 18.81 | N |
| ATOM | 6849 | CA | TYR | B | 409 | −7.075 | −31.413 | 108.120 | 1.00 | 18.74 | C |
| ATOM | 6850 | CB | TYR | B | 409 | −6.096 | −31.618 | 106.946 | 1.00 | 18.84 | C |
| ATOM | 6851 | CG | TYR | B | 409 | −5.734 | −30.338 | 106.199 | 1.00 | 19.99 | C |
| ATOM | 6852 | CD1 | TYR | B | 409 | −4.689 | −29.501 | 106.647 | 1.00 | 19.57 | C |
| ATOM | 6853 | CE1 | TYR | B | 409 | −4.368 | −28.326 | 105.974 | 1.00 | 19.09 | C |
| ATOM | 6854 | CZ | TYR | B | 409 | −5.085 | −27.970 | 104.845 | 1.00 | 19.76 | C |
| ATOM | 6855 | OH | TYR | B | 409 | −4.780 | −26.821 | 104.167 | 1.00 | 20.38 | O |
| ATOM | 6856 | CE2 | TYR | B | 409 | −6.113 | −28.786 | 104.368 | 1.00 | 20.83 | C |
| ATOM | 6857 | CD2 | TYR | B | 409 | −6.432 | −29.960 | 105.046 | 1.00 | 20.35 | C |
| ATOM | 6858 | C | TYR | B | 409 | −7.328 | −32.728 | 108.851 | 1.00 | 18.24 | C |
| ATOM | 6859 | O | TYR | B | 409 | −6.709 | −33.748 | 108.532 | 1.00 | 17.30 | O |
| ATOM | 6869 | N | PRO | B | 410 | −8.225 | −32.698 | 109.839 | 1.00 | 18.31 | N |
| ATOM | 6870 | CA | PRO | B | 410 | −8.457 | −33.860 | 110.712 | 1.00 | 18.54 | C |
| ATOM | 6871 | CB | PRO | B | 410 | −9.461 | −33.331 | 111.754 | 1.00 | 18.58 | C |
| ATOM | 6872 | CG | PRO | B | 410 | −10.159 | −32.187 | 111.087 | 1.00 | 18.74 | C |
| ATOM | 6873 | CD | PRO | B | 410 | −9.098 | −31.565 | 110.208 | 1.00 | 18.41 | C |
| ATOM | 6874 | C | PRO | B | 410 | −9.033 | −35.075 | 109.982 | 1.00 | 18.41 | C |
| ATOM | 6875 | O | PRO | B | 410 | −8.958 | −36.187 | 110.503 | 1.00 | 18.32 | O |
| ATOM | 6883 | N | HIS | B | 411 | −9.585 | −34.856 | 108.796 | 1.00 | 18.30 | N |
| ATOM | 6884 | CA | HIS | B | 411 | −10.212 | −35.918 | 108.038 | 1.00 | 18.54 | C |
| ATOM | 6885 | CB | HIS | B | 411 | −11.488 | −35.386 | 107.412 | 1.00 | 18.24 | C |
| ATOM | 6886 | CG | HIS | B | 411 | −12.570 | −35.143 | 108.402 | 1.00 | 16.79 | C |
| ATOM | 6887 | ND1 | HIS | B | 411 | −13.419 | −34.068 | 108.322 | 1.00 | 15.46 | N |
| ATOM | 6888 | CE1 | HIS | B | 411 | −14.282 | −34.120 | 109.317 | 1.00 | 17.37 | C |
| ATOM | 6889 | NE2 | HIS | B | 411 | −14.016 | −35.186 | 110.046 | 1.00 | 17.99 | N |
| ATOM | 6890 | CD2 | HIS | B | 411 | −12.946 | −35.842 | 109.493 | 1.00 | 16.72 | C |
| ATOM | 6891 | C | HIS | B | 411 | −9.306 | −36.527 | 106.974 | 1.00 | 19.31 | C |
| ATOM | 6892 | O | HIS | B | 411 | −9.725 | −37.418 | 106.241 | 1.00 | 19.35 | O |
| ATOM | 6901 | N | SER | B | 412 | −8.078 | −36.026 | 106.868 | 1.00 | 20.42 | N |
| ATOM | 6902 | CA | SER | B | 412 | −6.981 | −36.798 | 106.263 | 1.00 | 21.12 | C |
| ATOM | 6903 | CB | SER | B | 412 | −6.277 | −36.034 | 105.122 | 1.00 | 21.16 | C |
| ATOM | 6904 | OG | SER | B | 412 | −6.639 | −34.668 | 105.044 | 1.00 | 21.16 | O |
| ATOM | 6905 | C | SER | B | 412 | −6.000 | −37.214 | 107.382 | 1.00 | 21.39 | C |
| ATOM | 6906 | O | SER | B | 412 | −5.051 | −36.497 | 107.691 | 1.00 | 21.39 | O |
| ATOM | 6912 | N | GLY | B | 413 | −6.254 | −38.375 | 107.990 | 1.00 | 21.90 | N |
| ATOM | 6913 | CA | GLY | B | 413 | −5.530 | −38.799 | 109.183 | 1.00 | 22.37 | C |
| ATOM | 6914 | C | GLY | B | 413 | −4.092 | −39.228 | 108.927 | 1.00 | 22.44 | C |
| ATOM | 6915 | O | GLY | B | 413 | −3.331 | −39.513 | 109.857 | 1.00 | 22.29 | O |
| ATOM | 6919 | N | ASP | B | 414 | −3.733 | −39.253 | 107.648 | 1.00 | 22.70 | N |
| ATOM | 6920 | CA | ASP | B | 414 | −2.446 | −39.725 | 107.169 | 1.00 | 22.79 | C |
| ATOM | 6921 | CB | ASP | B | 414 | −2.708 | −40.861 | 106.184 | 1.00 | 23.34 | C |
| ATOM | 6922 | CG | ASP | B | 414 | −1.904 | −42.081 | 106.492 | 1.00 | 25.72 | C |
| ATOM | 6923 | OD1 | ASP | B | 414 | −0.677 | −41.927 | 106.757 | 1.00 | 25.79 | O |
| ATOM | 6924 | OD2 | ASP | B | 414 | −2.442 | −43.233 | 106.503 | 1.00 | 30.96 | O |
| ATOM | 6925 | C | ASP | B | 414 | −1.648 | −38.652 | 106.449 | 1.00 | 21.59 | C |
| ATOM | 6926 | O | ASP | B | 414 | −0.622 | −38.947 | 105.869 | 1.00 | 20.93 | O |
| ATOM | 6931 | N | LYS | B | 415 | −2.131 | −37.419 | 106.484 | 1.00 | 20.94 | N |
| ATOM | 6932 | CA | LYS | B | 415 | −1.593 | −36.366 | 105.650 | 1.00 | 20.82 | C |
| ATOM | 6933 | CB | LYS | B | 415 | −2.384 | −35.063 | 105.855 | 1.00 | 20.88 | C |
| ATOM | 6934 | CG | LYS | B | 415 | −1.814 | −33.818 | 105.138 | 1.00 | 22.14 | C |
| ATOM | 6935 | CD | LYS | B | 415 | −2.731 | −33.229 | 104.066 | 1.00 | 23.16 | C |
| ATOM | 6936 | CE | LYS | B | 415 | −3.147 | −31.794 | 104.345 | 1.00 | 22.70 | C |
| ATOM | 6937 | NZ | LYS | B | 415 | −2.831 | −30.825 | 103.256 | 1.00 | 20.39 | N |
| ATOM | 6938 | C | LYS | B | 415 | −0.106 | −36.169 | 105.960 | 1.00 | 20.56 | C |
| ATOM | 6939 | O | LYS | B | 415 | 0.697 | −36.004 | 105.064 | 1.00 | 19.34 | O |
| ATOM | 6953 | N | PHE | B | 416 | 0.233 | −36.195 | 107.242 | 1.00 | 20.78 | N |
| ATOM | 6954 | CA | PHE | B | 416 | 1.593 | −35.999 | 107.695 | 1.00 | 20.83 | C |
| ATOM | 6955 | CB | PHE | B | 416 | 1.650 | −36.191 | 109.204 | 1.00 | 20.97 | C |
| ATOM | 6956 | CG | PHE | B | 416 | 3.024 | −36.143 | 109.759 | 1.00 | 20.30 | C |
| ATOM | 6957 | CD1 | PHE | B | 416 | 3.746 | −34.967 | 109.729 | 1.00 | 20.06 | C |
| ATOM | 6958 | CE1 | PHE | B | 416 | 5.024 | −34.907 | 110.229 | 1.00 | 21.54 | C |
| ATOM | 6959 | CZ | PHE | B | 416 | 5.595 | −36.043 | 110.790 | 1.00 | 24.11 | C |
| ATOM | 6960 | CE2 | PHE | B | 416 | 4.871 | −37.236 | 110.833 | 1.00 | 22.95 | C |
| ATOM | 6961 | CD2 | PHE | B | 416 | 3.598 | −37.276 | 110.308 | 1.00 | 21.71 | C |
| ATOM | 6962 | C | PHE | B | 416 | 2.523 | −37.002 | 107.041 | 1.00 | 21.35 | C |
| ATOM | 6963 | O | PHE | B | 416 | 3.546 | −36.634 | 106.474 | 1.00 | 20.47 | O |
| ATOM | 6973 | N | GLN | B | 417 | 2.159 | −38.275 | 107.135 | 1.00 | 21.61 | N |
| ATOM | 6974 | CA | GLN | B | 417 | 2.978 | −39.329 | 106.555 | 1.00 | 22.38 | C |
| ATOM | 6975 | CB | GLN | B | 417 | 2.482 | −40.713 | 106.983 | 1.00 | 22.46 | C |
| ATOM | 6976 | CG | GLN | B | 417 | 3.524 | −41.511 | 107.759 | 1.00 | 25.00 | C |
| ATOM | 6977 | CD | GLN | B | 417 | 3.746 | −41.019 | 109.213 | 1.00 | 27.27 | C |
| ATOM | 6978 | OE1 | GLN | B | 417 | 2.998 | −41.407 | 110.138 | 1.00 | 26.95 | O |
| ATOM | 6979 | NE2 | GLN | B | 417 | 4.784 | −40.187 | 109.412 | 1.00 | 26.51 | N |
| ATOM | 6980 | C | GLN | B | 417 | 3.052 | −39.255 | 105.030 | 1.00 | 22.54 | C |
| ATOM | 6981 | O | GLN | B | 417 | 4.107 | −39.500 | 104.446 | 1.00 | 23.17 | O |
| ATOM | 6990 | N | GLN | B | 418 | 1.932 | −38.942 | 104.385 | 1.00 | 22.40 | N |
| ATOM | 6991 | CA | GLN | B | 418 | 1.885 | −38.827 | 102.931 | 1.00 | 22.19 | C |
| ATOM | 6992 | CB | GLN | B | 418 | 0.435 | −38.629 | 102.464 | 1.00 | 22.16 | C |
| ATOM | 6993 | CG | GLN | B | 418 | −0.476 | −39.856 | 102.700 | 1.00 | 24.07 | C |
| ATOM | 6994 | CD | GLN | B | 418 | 0.136 | −41.194 | 102.215 | 1.00 | 27.05 | C |
| ATOM | 6995 | OE1 | GLN | B | 418 | 0.512 | −41.327 | 101.032 | 1.00 | 28.99 | O |
| ATOM | 6996 | NE2 | GLN | B | 418 | 0.220 | −42.185 | 103.121 | 1.00 | 26.31 | N |
| ATOM | 6997 | C | GLN | B | 418 | 2.784 | −37.688 | 102.443 | 1.00 | 21.63 | C |
| ATOM | 6998 | O | GLN | B | 418 | 3.389 | −37.780 | 101.402 | 1.00 | 21.00 | O |
| ATOM | 7007 | N | LEU | B | 419 | 2.879 | −36.630 | 103.230 | 1.00 | 21.62 | N |
| ATOM | 7008 | CA | LEU | B | 419 | 3.716 | −35.480 | 102.899 | 1.00 | 21.95 | C |
| ATOM | 7009 | CB | LEU | B | 419 | 3.324 | −34.260 | 103.755 | 1.00 | 21.35 | C |
| ATOM | 7010 | CG | LEU | B | 419 | 2.034 | −33.521 | 103.379 | 1.00 | 20.69 | C |
| ATOM | 7011 | CD1 | LEU | B | 419 | 1.764 | −32.388 | 104.401 | 1.00 | 20.62 | C |
| ATOM | 7012 | CD2 | LEU | B | 419 | 2.085 | −32.968 | 101.955 | 1.00 | 19.91 | C |
| ATOM | 7013 | C | LEU | B | 419 | 5.207 | −35.808 | 103.055 | 1.00 | 22.13 | C |
| ATOM | 7014 | O | LEU | B | 419 | 6.030 | −35.384 | 102.249 | 1.00 | 22.28 | O |
| ATOM | 7026 | N | LEU | B | 420 | 5.540 | −36.552 | 104.099 | 1.00 | 22.38 | N |
| ATOM | 7027 | CA | LEU | B | 420 | 6.895 | −37.028 | 104.301 | 1.00 | 22.58 | C |
| ATOM | 7028 | CB | LEU | B | 420 | 7.008 | −37.730 | 105.650 | 1.00 | 22.77 | C |
| ATOM | 7029 | CG | LEU | B | 420 | 6.987 | −36.848 | 106.898 | 1.00 | 22.87 | C |
| ATOM | 7030 | CD1 | LEU | B | 420 | 7.395 | −37.687 | 108.084 | 1.00 | 22.76 | C |
| ATOM | 7031 | CD2 | LEU | B | 420 | 7.888 | −35.606 | 106.768 | 1.00 | 22.43 | C |
| ATOM | 7032 | C | LEU | B | 420 | 7.328 | −37.983 | 103.195 | 1.00 | 22.86 | C |
| ATOM | 7033 | O | LEU | B | 420 | 8.488 | −37.985 | 102.809 | 1.00 | 23.16 | O |
| ATOM | 7045 | N | LEU | B | 421 | 6.401 | −38.800 | 102.694 | 1.00 | 22.70 | N |
| ATOM | 7046 | CA | LEU | B | 421 | 6.693 | −39.703 | 101.584 | 1.00 | 22.68 | C |
| ATOM | 7047 | CB | LEU | B | 421 | 5.518 | −40.669 | 101.379 | 1.00 | 22.74 | C |
| ATOM | 7048 | CG | LEU | B | 421 | 5.511 | −41.608 | 100.162 | 1.00 | 23.68 | C |
| ATOM | 7049 | CD1 | LEU | B | 421 | 6.720 | −42.543 | 100.190 | 1.00 | 24.19 | C |
| ATOM | 7050 | CD2 | LEU | B | 421 | 4.208 | −42.439 | 100.090 | 1.00 | 23.40 | C |
| ATOM | 7051 | C | LEU | B | 421 | 7.012 | −38.893 | 100.306 | 1.00 | 22.82 | C |
| ATOM | 7052 | O | LEU | B | 421 | 7.884 | −39.256 | 99.506 | 1.00 | 22.49 | O |
| ATOM | 7064 | N | CYS | B | 422 | 6.320 | −37.773 | 100.137 | 1.00 | 22.82 | N |
| ATOM | 7065 | CA | CYS | B | 422 | 6.593 | −36.874 | 99.027 | 1.00 | 22.67 | C |
| ATOM | 7066 | CB | CYS | B | 422 | 5.595 | −35.716 | 99.018 | 1.00 | 22.94 | C |
| ATOM | 7067 | SG | CYS | B | 422 | 3.923 | −36.151 | 98.517 | 1.00 | 20.83 | S |
| ATOM | 7068 | C | CYS | B | 422 | 8.024 | −36.332 | 99.079 | 1.00 | 22.51 | C |
| ATOM | 7069 | O | CYS | B | 422 | 8.622 | −36.081 | 98.050 | 1.00 | 22.33 | O |
| ATOM | 7075 | N | LEU | B | 423 | 8.532 | −36.136 | 100.288 | 1.00 | 22.32 | N |
| ATOM | 7076 | CA | LEU | B | 423 | 9.907 | −35.736 | 100.517 | 1.00 | 22.50 | C |
| ATOM | 7077 | CB | LEU | B | 423 | 10.120 | −35.401 | 101.998 | 1.00 | 22.66 | C |
| ATOM | 7078 | CG | LEU | B | 423 | 10.104 | −33.929 | 102.403 | 1.00 | 23.93 | C |
| ATOM | 7079 | CD1 | LEU | B | 423 | 9.182 | −33.051 | 101.514 | 1.00 | 25.23 | C |
| ATOM | 7080 | CD2 | LEU | B | 423 | 9.770 | −33.770 | 103.879 | 1.00 | 23.91 | C |
| ATOM | 7081 | C | LEU | B | 423 | 10.894 | −36.821 | 100.074 | 1.00 | 22.59 | C |
| ATOM | 7082 | O | LEU | B | 423 | 11.970 | −36.511 | 99.575 | 1.00 | 22.73 | O |
| ATOM | 7094 | N | VAL | B | 424 | 10.532 | −38.086 | 100.232 | 1.00 | 22.19 | N |
| ATOM | 7095 | CA | VAL | B | 424 | 11.363 | −39.169 | 99.732 | 1.00 | 22.31 | C |
| ATOM | 7096 | CB | VAL | B | 424 | 10.786 | −40.558 | 100.100 | 1.00 | 22.12 | C |
| ATOM | 7097 | CG1 | VAL | B | 424 | 11.561 | −41.678 | 99.434 | 1.00 | 22.07 | C |
| ATOM | 7098 | CG2 | VAL | B | 424 | 10.785 | −40.769 | 101.612 | 1.00 | 22.49 | C |
| ATOM | 7099 | C | VAL | B | 424 | 11.495 | −39.020 | 98.215 | 1.00 | 22.77 | C |
| ATOM | 7100 | O | VAL | B | 424 | 12.580 | −39.155 | 97.648 | 1.00 | 22.70 | O |
| ATOM | 7110 | N | GLU | B | 425 | 10.379 | −38.708 | 97.574 | 1.00 | 23.11 | N |
| ATOM | 7111 | CA | GLU | B | 425 | 10.335 | −38.580 | 96.138 | 1.00 | 23.64 | C |
| ATOM | 7112 | CB | GLU | B | 425 | 8.874 | −38.558 | 95.626 | 1.00 | 24.51 | C |
| ATOM | 7113 | CG | GLU | B | 425 | 8.692 | −38.352 | 94.103 | 1.00 | 27.05 | C |
| ATOM | 7114 | CD | GLU | B | 425 | 9.340 | −39.443 | 93.216 | 1.00 | 30.11 | C |
| ATOM | 7115 | OE1 | GLU | B | 425 | 9.811 | −40.489 | 93.741 | 1.00 | 31.04 | O |
| ATOM | 7116 | OE2 | GLU | B | 425 | 9.383 | −39.252 | 91.972 | 1.00 | 31.28 | O |
| ATOM | 7117 | C | GLU | B | 425 | 11.090 | −37.362 | 95.674 | 1.00 | 22.54 | C |
| ATOM | 7118 | O | GLU | B | 425 | 11.609 | −37.375 | 94.593 | 1.00 | 22.71 | O |
| ATOM | 7125 | N | VAL | B | 426 | 11.145 | −36.315 | 96.481 | 1.00 | 22.34 | N |
| ATOM | 7126 | CA | VAL | B | 426 | 11.919 | −35.123 | 96.147 | 1.00 | 22.46 | C |
| ATOM | 7127 | CB | VAL | B | 426 | 11.626 | −33.942 | 97.096 | 1.00 | 22.32 | C |
| ATOM | 7128 | CG1 | VAL | B | 426 | 12.675 | −32.826 | 96.968 | 1.00 | 21.39 | C |
| ATOM | 7129 | CG2 | VAL | B | 426 | 10.226 | −33.409 | 96.851 | 1.00 | 23.48 | C |
| ATOM | 7130 | C | VAL | B | 426 | 13.405 | −35.447 | 96.189 | 1.00 | 22.58 | C |
| ATOM | 7131 | O | VAL | B | 426 | 14.163 | −34.940 | 95.372 | 1.00 | 23.48 | O |
| ATOM | 7141 | N | ARG | B | 427 | 13.816 | −36.286 | 97.129 | 1.00 | 22.61 | N |
| ATOM | 7142 | CA | ARG | B | 427 | 15.193 | −36.762 | 97.177 | 1.00 | 23.27 | C |
| ATOM | 7143 | CB | ARG | B | 427 | 15.458 | −37.517 | 98.485 | 1.00 | 23.29 | C |
| ATOM | 7144 | CG | ARG | B | 427 | 16.939 | −37.668 | 98.832 | 1.00 | 27.00 | C |
| ATOM | 7145 | CD | ARG | B | 427 | 17.282 | −38.927 | 99.634 | 1.00 | 30.09 | C |
| ATOM | 7146 | NE | ARG | B | 427 | 18.693 | −39.019 | 100.042 | 1.00 | 32.26 | N |
| ATOM | 7147 | CZ | ARG | B | 427 | 19.714 | −39.352 | 99.241 | 1.00 | 33.99 | C |
| ATOM | 7148 | NH1 | ARG | B | 427 | 19.516 | −39.612 | 97.948 | 1.00 | 35.23 | N |
| ATOM | 7149 | NH2 | ARG | B | 427 | 20.956 | −39.407 | 99.732 | 1.00 | 34.24 | N |
| ATOM | 7150 | C | ARG | B | 427 | 15.575 | −37.603 | 95.919 | 1.00 | 22.55 | C |
| ATOM | 7151 | O | ARG | B | 427 | 16.640 | −37.408 | 95.366 | 1.00 | 21.84 | O |
| ATOM | 7165 | N | ALA | B | 428 | 14.689 | −38.488 | 95.465 | 1.00 | 22.27 | N |
| ATOM | 7166 | CA | ALA | B | 428 | 14.887 | −39.271 | 94.239 | 1.00 | 22.31 | C |
| ATOM | 7167 | CB | ALA | B | 428 | 13.740 | −40.261 | 94.074 | 1.00 | 22.19 | C |
| ATOM | 7168 | C | ALA | B | 428 | 15.006 | −38.401 | 92.969 | 1.00 | 22.76 | C |
| ATOM | 7169 | O | ALA | B | 428 | 15.850 | −38.639 | 92.106 | 1.00 | 22.20 | O |
| ATOM | 7175 | N | LEU | B | 429 | 14.129 | −37.403 | 92.878 | 1.00 | 23.53 | N |
| ATOM | 7176 | CA | LEU | B | 429 | 14.125 | −36.412 | 91.819 | 1.00 | 23.79 | C |
| ATOM | 7177 | CB | LEU | B | 429 | 13.000 | −35.408 | 92.028 | 1.00 | 23.98 | C |
| ATOM | 7178 | CG | LEU | B | 429 | 11.689 | −35.789 | 91.375 | 1.00 | 25.98 | C |
| ATOM | 7179 | CD1 | LEU | B | 429 | 10.590 | −34.845 | 91.818 | 1.00 | 26.36 | C |
| ATOM | 7180 | CD2 | LEU | B | 429 | 11.870 | −35.763 | 89.852 | 1.00 | 28.45 | C |
| ATOM | 7181 | C | LEU | B | 429 | 15.394 | −35.629 | 91.818 | 1.00 | 23.51 | C |
| ATOM | 7182 | O | LEU | B | 429 | 15.915 | −35.309 | 90.757 | 1.00 | 23.86 | O |
| ATOM | 7194 | N | SER | B | 430 | 15.853 | −35.284 | 93.014 | 1.00 | 23.31 | N |
| ATOM | 7195 | CA | SER | B | 430 | 17.080 | −34.512 | 93.214 | 1.00 | 23.04 | C |
| ATOM | 7196 | CB | SER | B | 430 | 17.259 | −34.189 | 94.674 | 1.00 | 22.85 | C |
| ATOM | 7197 | OG | SER | B | 430 | 16.253 | −33.262 | 94.991 | 1.00 | 26.94 | O |
| ATOM | 7198 | C | SER | B | 430 | 18.313 | −35.209 | 92.749 | 1.00 | 22.50 | C |
| ATOM | 7199 | O | SER | B | 430 | 19.239 | −34.564 | 92.300 | 1.00 | 22.33 | O |
| ATOM | 7205 | N | MET | B | 431 | 18.327 | −36.525 | 92.872 | 1.00 | 22.75 | N |
| ATOM | 7206 | CA | MET | B | 431 | 19.450 | −37.310 | 92.418 | 1.00 | 23.05 | C |
| ATOM | 7207 | CB | MET | B | 431 | 19.480 | −38.704 | 93.055 | 1.00 | 23.86 | C |
| ATOM | 7208 | CG | MET | B | 431 | 19.618 | −38.729 | 94.603 | 1.00 | 27.56 | C |
| ATOM | 7209 | SD | MET | B | 431 | 21.120 | −37.925 | 95.389 | 1.00 | 35.11 | S |
| ATOM | 7210 | CE | MET | B | 431 | 20.084 | −36.525 | 96.354 | 1.00 | 32.67 | C |
| ATOM | 7211 | C | MET | B | 431 | 19.453 | −37.370 | 90.897 | 1.00 | 22.10 | C |
| ATOM | 7212 | O | MET | B | 431 | 20.498 | −37.184 | 90.304 | 1.00 | 21.56 | O |
| ATOM | 7222 | N | GLN | B | 432 | 18.302 | −37.587 | 90.261 | 1.00 | 21.51 | N |
| ATOM | 7223 | CA | GLN | B | 432 | 18.236 | −37.563 | 88.792 | 1.00 | 21.34 | C |
| ATOM | 7224 | CB | GLN | B | 432 | 16.811 | −37.783 | 88.298 | 1.00 | 22.12 | C |
| ATOM | 7225 | CG | GLN | B | 432 | 16.379 | −39.214 | 88.008 | 1.00 | 24.82 | C |
| ATOM | 7226 | CD | GLN | B | 432 | 14.895 | −39.261 | 87.673 | 1.00 | 27.87 | C |
| ATOM | 7227 | OE1 | GLN | B | 432 | 14.107 | −39.859 | 88.570 | 1.00 | 29.87 | O |
| ATOM | 7228 | NE2 | GLN | B | 432 | 14.460 | −38.736 | 86.622 | 1.00 | 30.12 | N |
| ATOM | 7229 | C | GLN | B | 432 | 18.663 | −36.217 | 88.227 | 1.00 | 20.10 | C |
| ATOM | 7230 | O | GLN | B | 432 | 19.226 | −36.145 | 87.154 | 1.00 | 20.01 | O |
| ATOM | 7239 | N | ALA | B | 433 | 18.305 | −35.154 | 88.926 | 1.00 | 19.02 | N |
| ATOM | 7240 | CA | ALA | B | 433 | 18.659 | −33.802 | 88.546 | 1.00 | 18.88 | C |
| ATOM | 7241 | CB | ALA | B | 433 | 17.923 | −32.774 | 89.432 | 1.00 | 18.80 | C |
| ATOM | 7242 | C | ALA | B | 433 | 20.149 | −33.595 | 88.637 | 1.00 | 18.10 | C |
| ATOM | 7243 | O | ALA | B | 433 | 20.723 | −32.985 | 87.772 | 1.00 | 18.11 | O |
| ATOM | 7249 | N | LYS | B | 434 | 20.762 | −34.079 | 89.708 | 1.00 | 17.95 | N |
| ATOM | 7250 | CA | LYS | B | 434 | 22.208 | −34.084 | 89.842 | 1.00 | 17.73 | C |
| ATOM | 7251 | CB | LYS | B | 434 | 22.616 | −34.783 | 91.134 | 1.00 | 18.12 | C |
| ATOM | 7252 | CG | LYS | B | 434 | 22.520 | −33.926 | 92.368 | 1.00 | 20.50 | C |
| ATOM | 7253 | CD | LYS | B | 434 | 22.868 | −34.756 | 93.584 | 1.00 | 23.01 | C |
| ATOM | 7254 | CE | LYS | B | 434 | 23.195 | −33.910 | 94.765 | 1.00 | 25.51 | C |
| ATOM | 7255 | NZ | LYS | B | 434 | 23.192 | −34.712 | 96.017 | 1.00 | 27.93 | N |
| ATOM | 7256 | C | LYS | B | 434 | 22.887 | −34.797 | 88.685 | 1.00 | 16.47 | C |
| ATOM | 7257 | O | LYS | B | 434 | 23.922 | −34.369 | 88.232 | 1.00 | 15.82 | O |
| ATOM | 7271 | N | GLU | B | 435 | 22.313 | −35.902 | 88.244 | 1.00 | 15.85 | N |
| ATOM | 7272 | CA | GLU | B | 435 | 22.839 | −36.657 | 87.127 | 1.00 | 15.85 | C |
| ATOM | 7273 | CB | GLU | B | 435 | 22.143 | −38.009 | 87.025 | 1.00 | 15.55 | C |
| ATOM | 7274 | CG | GLU | B | 435 | 22.351 | −38.843 | 88.253 | 1.00 | 15.98 | C |
| ATOM | 7275 | CD | GLU | B | 435 | 21.851 | −40.243 | 88.091 | 1.00 | 17.57 | C |
| ATOM | 7276 | OE1 | GLU | B | 435 | 21.214 | −40.733 | 88.996 | 1.00 | 20.15 | O |
| ATOM | 7277 | OE2 | GLU | B | 435 | 22.141 | −40.888 | 87.082 | 1.00 | 23.10 | O |
| ATOM | 7278 | C | GLU | B | 435 | 22.719 | −35.897 | 85.803 | 1.00 | 15.78 | C |
| ATOM | 7279 | O | GLU | B | 435 | 23.583 | −36.028 | 84.943 | 1.00 | 15.95 | O |
| ATOM | 7286 | N | TYR | B | 436 | 21.659 | −35.108 | 85.660 | 1.00 | 16.29 | N |
| ATOM | 7287 | CA | TYR | B | 436 | 21.477 | −34.231 | 84.514 | 1.00 | 16.80 | C |
| ATOM | 7288 | CB | TYR | B | 436 | 20.115 | −33.530 | 84.574 | 1.00 | 16.95 | C |
| ATOM | 7289 | CG | TYR | B | 436 | 19.949 | −32.462 | 83.512 | 1.00 | 17.51 | C |
| ATOM | 7290 | CD1 | TYR | B | 436 | 19.787 | −32.797 | 82.177 | 1.00 | 17.21 | C |
| ATOM | 7291 | CE1 | TYR | B | 436 | 19.655 | −31.808 | 81.204 | 1.00 | 17.44 | C |
| ATOM | 7292 | CZ | TYR | B | 436 | 19.691 | −30.479 | 81.564 | 1.00 | 15.57 | C |
| ATOM | 7293 | OH | TYR | B | 436 | 19.578 | −29.493 | 80.622 | 1.00 | 11.65 | O |
| ATOM | 7294 | CE2 | TYR | B | 436 | 19.861 | −30.136 | 82.869 | 1.00 | 17.56 | C |
| ATOM | 7295 | CD2 | TYR | B | 436 | 19.990 | −31.121 | 83.839 | 1.00 | 17.47 | C |
| ATOM | 7296 | C | TYR | B | 436 | 22.583 | −33.194 | 84.465 | 1.00 | 16.61 | C |
| ATOM | 7297 | O | TYR | B | 436 | 23.205 | −32.999 | 83.440 | 1.00 | 15.88 | O |
| ATOM | 7307 | N | LEU | B | 437 | 22.807 | −32.537 | 85.597 | 1.00 | 16.76 | N |
| ATOM | 7308 | CA | LEU | B | 437 | 23.881 | −31.562 | 85.760 | 1.00 | 17.13 | C |
| ATOM | 7309 | CB | LEU | B | 437 | 23.883 | −31.019 | 87.188 | 1.00 | 17.44 | C |
| ATOM | 7310 | CG | LEU | B | 437 | 22.784 | −30.035 | 87.506 | 1.00 | 20.73 | C |
| ATOM | 7311 | CD1 | LEU | B | 437 | 22.836 | −29.720 | 88.995 | 1.00 | 23.50 | C |
| ATOM | 7312 | CD2 | LEU | B | 437 | 22.939 | −28.764 | 86.653 | 1.00 | 22.47 | C |
| ATOM | 7313 | C | LEU | B | 437 | 25.259 | −32.107 | 85.490 | 1.00 | 16.11 | C |
| ATOM | 7314 | O | LEU | B | 437 | 26.078 | −31.416 | 84.921 | 1.00 | 16.51 | O |
| ATOM | 7326 | N | TYR | B | 438 | 25.529 | −33.323 | 85.947 | 1.00 | 15.30 | N |
| ATOM | 7327 | CA | TYR | B | 438 | 26.831 | −33.948 | 85.752 | 1.00 | 14.80 | C |
| ATOM | 7328 | CB | TYR | B | 438 | 26.927 | −35.233 | 86.578 | 1.00 | 14.49 | C |
| ATOM | 7329 | CG | TYR | B | 438 | 28.236 | −35.957 | 86.479 | 1.00 | 11.52 | C |
| ATOM | 7330 | CD1 | TYR | B | 438 | 29.379 | −35.454 | 87.075 | 1.00 | 9.80 | C |
| ATOM | 7331 | CE1 | TYR | B | 438 | 30.584 | −36.119 | 86.988 | 1.00 | 8.50 | C |
| ATOM | 7332 | CZ | TYR | B | 438 | 30.657 | −37.314 | 86.315 | 1.00 | 7.69 | C |
| ATOM | 7333 | OH | TYR | B | 438 | 31.826 | −37.989 | 86.237 | 1.00 | 4.74 | O |
| ATOM | 7334 | CE2 | TYR | B | 438 | 29.544 | −37.836 | 85.719 | 1.00 | 9.89 | C |
| ATOM | 7335 | CD2 | TYR | B | 438 | 28.329 | −37.153 | 85.807 | 1.00 | 10.18 | C |
| ATOM | 7336 | C | TYR | B | 438 | 27.076 | −34.229 | 84.266 | 1.00 | 14.44 | C |
| ATOM | 7337 | O | TYR | B | 438 | 28.175 | −34.005 | 83.766 | 1.00 | 13.05 | O |
| ATOM | 7347 | N | HIS | B | 439 | 26.036 | −34.701 | 83.583 | 1.00 | 14.58 | N |
| ATOM | 7348 | CA | HIS | B | 439 | 26.062 | −34.912 | 82.132 | 1.00 | 15.49 | C |
| ATOM | 7349 | CB | HIS | B | 439 | 24.714 | −35.455 | 81.637 | 1.00 | 15.49 | C |
| ATOM | 7350 | CG | HIS | B | 439 | 24.567 | −35.424 | 80.153 | 1.00 | 16.84 | C |
| ATOM | 7351 | ND1 | HIS | B | 439 | 25.173 | −36.345 | 79.327 | 1.00 | 19.78 | N |
| ATOM | 7352 | CE1 | HIS | B | 439 | 24.887 | −36.061 | 78.069 | 1.00 | 21.07 | C |
| ATOM | 7353 | NE2 | HIS | B | 439 | 24.120 | −34.984 | 78.050 | 1.00 | 20.71 | N |
| ATOM | 7354 | CD2 | HIS | B | 439 | 23.912 | −34.562 | 79.342 | 1.00 | 19.48 | C |
| ATOM | 7355 | C | HIS | B | 439 | 26.404 | −33.631 | 81.364 | 1.00 | 15.61 | C |
| ATOM | 7356 | O | HIS | B | 439 | 27.259 | −33.638 | 80.483 | 1.00 | 15.55 | O |
| ATOM | 7365 | N | LYS | B | 440 | 25.712 | −32.557 | 81.721 | 1.00 | 15.84 | N |
| ATOM | 7366 | CA | LYS | B | 440 | 25.914 | −31.235 | 81.148 | 1.00 | 16.87 | C |
| ATOM | 7367 | CB | LYS | B | 440 | 24.929 | −30.262 | 81.777 | 1.00 | 16.99 | C |
| ATOM | 7368 | CG | LYS | B | 440 | 23.563 | −30.082 | 81.138 | 1.00 | 18.92 | C |
| ATOM | 7369 | CD | LYS | B | 440 | 23.194 | −30.887 | 79.889 | 1.00 | 21.22 | C |
| ATOM | 7370 | CE | LYS | B | 440 | 22.590 | −29.936 | 78.838 | 1.00 | 22.38 | C |
| ATOM | 7371 | NZ | LYS | B | 440 | 22.180 | −30.579 | 77.547 | 1.00 | 23.99 | N |
| ATOM | 7372 | C | LYS | B | 440 | 27.320 | −30.724 | 81.410 | 1.00 | 16.85 | C |
| ATOM | 7373 | O | LYS | B | 440 | 27.990 | −30.207 | 80.527 | 1.00 | 16.61 | O |
| ATOM | 7387 | N | HIS | B | 441 | 27.752 | −30.891 | 82.647 | 1.00 | 17.02 | N |
| ATOM | 7388 | CA | HIS | B | 441 | 29.100 | −30.563 | 83.076 | 1.00 | 17.03 | C |
| ATOM | 7389 | CB | HIS | B | 441 | 29.228 | −30.921 | 84.560 | 1.00 | 17.15 | C |
| ATOM | 7390 | CG | HIS | B | 441 | 30.628 | −30.922 | 85.050 | 1.00 | 16.74 | C |
| ATOM | 7391 | ND1 | HIS | B | 441 | 31.277 | −29.770 | 85.428 | 1.00 | 17.40 | N |
| ATOM | 7392 | CE1 | HIS | B | 441 | 32.510 | −30.070 | 85.801 | 1.00 | 18.55 | C |
| ATOM | 7393 | NE2 | HIS | B | 441 | 32.687 | −31.372 | 85.659 | 1.00 | 18.05 | N |
| ATOM | 7394 | CD2 | HIS | B | 441 | 31.521 | −31.929 | 85.196 | 1.00 | 18.60 | C |
| ATOM | 7395 | C | HIS | B | 441 | 30.217 | −31.261 | 82.284 | 1.00 | 17.10 | C |
| ATOM | 7396 | O | HIS | B | 441 | 31.256 | −30.646 | 81.989 | 1.00 | 16.96 | O |
| ATOM | 7405 | N | LEU | B | 442 | 30.028 | −32.543 | 81.968 | 1.00 | 16.97 | N |
| ATOM | 7406 | CA | LEU | B | 442 | 31.078 | −33.323 | 81.309 | 1.00 | 17.19 | C |
| ATOM | 7407 | CB | LEU | B | 442 | 30.836 | −34.838 | 81.416 | 1.00 | 16.69 | C |
| ATOM | 7408 | CG | LEU | B | 442 | 31.123 | −35.537 | 82.742 | 1.00 | 15.56 | C |
| ATOM | 7409 | CD1 | LEU | B | 442 | 30.754 | −36.989 | 82.597 | 1.00 | 15.35 | C |
| ATOM | 7410 | CD2 | LEU | B | 442 | 32.562 | −35.397 | 83.173 | 1.00 | 14.70 | C |
| ATOM | 7411 | C | LEU | B | 442 | 31.167 | −32.907 | 79.848 | 1.00 | 17.78 | C |
| ATOM | 7412 | O | LEU | B | 442 | 32.244 | −32.924 | 79.263 | 1.00 | 17.31 | O |
| ATOM | 7424 | N | GLY | B | 443 | 30.023 | −32.517 | 79.286 | 1.00 | 19.00 | N |
| ATOM | 7425 | CA | GLY | B | 443 | 29.939 | −32.036 | 77.920 | 1.00 | 19.92 | C |
| ATOM | 7426 | C | GLY | B | 443 | 30.392 | −30.599 | 77.774 | 1.00 | 20.64 | C |
| ATOM | 7427 | O | GLY | B | 443 | 30.306 | −30.049 | 76.685 | 1.00 | 20.88 | O |
| ATOM | 7431 | N | ASN | B | 444 | 30.867 | −30.010 | 78.872 | 1.00 | 21.66 | N |
| ATOM | 7432 | CA | ASN | B | 444 | 31.364 | −28.636 | 78.935 | 1.00 | 22.75 | C |
| ATOM | 7433 | CB | ASN | B | 444 | 32.661 | −28.480 | 78.126 | 1.00 | 23.21 | C |
| ATOM | 7434 | CG | ASN | B | 444 | 33.860 | −28.232 | 79.014 | 1.00 | 25.30 | C |
| ATOM | 7435 | OD1 | ASN | B | 444 | 33.759 | −27.521 | 80.027 | 1.00 | 28.04 | O |
| ATOM | 7436 | ND2 | ASN | B | 444 | 35.007 | −28.818 | 78.651 | 1.00 | 27.89 | N |
| ATOM | 7437 | C | ASN | B | 444 | 30.326 | −27.589 | 78.541 | 1.00 | 22.95 | C |
| ATOM | 7438 | O | ASN | B | 444 | 30.648 | −26.532 | 78.011 | 1.00 | 23.14 | O |
| ATOM | 7445 | N | GLU | B | 445 | 29.080 | −27.889 | 78.862 | 1.00 | 23.41 | N |
| ATOM | 7446 | CA | GLU | B | 445 | 27.931 | −27.101 | 78.432 | 1.00 | 23.88 | C |
| ATOM | 7447 | CB | GLU | B | 445 | 26.800 | −28.049 | 78.022 | 1.00 | 24.02 | C |
| ATOM | 7448 | CG | GLU | B | 445 | 27.298 | −29.218 | 77.178 | 1.00 | 25.96 | C |
| ATOM | 7449 | CD | GLU | B | 445 | 26.219 | −29.911 | 76.365 | 1.00 | 29.01 | C |
| ATOM | 7450 | OE1 | GLU | B | 445 | 25.403 | −30.668 | 76.950 | 1.00 | 29.85 | O |
| ATOM | 7451 | OE2 | GLU | B | 445 | 26.212 | −29.717 | 75.127 | 1.00 | 32.33 | O |
| ATOM | 7452 | C | GLU | B | 445 | 27.459 | −26.120 | 79.505 | 1.00 | 23.74 | C |
| ATOM | 7453 | O | GLU | B | 445 | 26.709 | −25.191 | 79.202 | 1.00 | 24.18 | O |
| ATOM | 7460 | N | MET | B | 446 | 27.893 | −26.326 | 80.749 | 1.00 | 23.56 | N |
| ATOM | 7461 | CA | MET | B | 446 | 27.545 | −25.419 | 81.845 | 1.00 | 23.61 | C |
| ATOM | 7462 | CB | MET | B | 446 | 27.745 | −26.071 | 83.223 | 1.00 | 23.35 | C |
| ATOM | 7463 | CG | MET | B | 446 | 26.910 | −27.314 | 83.476 | 1.00 | 22.74 | C |
| ATOM | 7464 | SD | MET | B | 446 | 25.147 | −27.053 | 83.444 | 1.00 | 23.47 | S |
| ATOM | 7465 | CE | MET | B | 446 | 24.926 | −26.049 | 84.899 | 1.00 | 23.47 | C |
| ATOM | 7466 | C | MET | B | 446 | 28.384 | −24.158 | 81.789 | 1.00 | 23.64 | C |
| ATOM | 7467 | O | MET | B | 446 | 29.538 | −24.204 | 81.381 | 1.00 | 23.28 | O |
| ATOM | 7477 | N | PRO | B | 447 | 27.805 | −23.037 | 82.213 | 1.00 | 24.28 | N |
| ATOM | 7478 | CA | PRO | B | 447 | 28.568 | −21.798 | 82.413 | 1.00 | 24.88 | C |
| ATOM | 7479 | CB | PRO | B | 447 | 27.531 | −20.855 | 83.023 | 1.00 | 24.82 | C |
| ATOM | 7480 | CG | PRO | B | 447 | 26.228 | −21.362 | 82.532 | 1.00 | 24.37 | C |
| ATOM | 7481 | CD | PRO | B | 447 | 26.376 | −22.847 | 82.519 | 1.00 | 24.16 | C |
| ATOM | 7482 | C | PRO | B | 447 | 29.713 | −22.007 | 83.393 | 1.00 | 25.43 | C |
| ATOM | 7483 | O | PRO | B | 447 | 29.571 | −22.812 | 84.316 | 1.00 | 25.71 | O |
| ATOM | 7491 | N | ARG | B | 448 | 30.817 | −21.291 | 83.212 | 1.00 | 26.30 | N |
| ATOM | 7492 | CA | ARG | B | 448 | 31.995 | −21.513 | 84.056 | 1.00 | 26.79 | C |
| ATOM | 7493 | CB | ARG | B | 448 | 33.284 | −20.978 | 83.393 | 1.00 | 27.35 | C |
| ATOM | 7494 | CG | ARG | B | 448 | 34.559 | −21.889 | 83.596 | 1.00 | 29.10 | C |
| ATOM | 7495 | CD | ARG | B | 448 | 34.620 | −22.642 | 84.983 | 1.00 | 31.72 | C |
| ATOM | 7496 | NE | ARG | B | 448 | 35.755 | −23.567 | 85.180 | 1.00 | 33.20 | N |
| ATOM | 7497 | CZ | ARG | B | 448 | 35.858 | −24.459 | 86.198 | 1.00 | 32.57 | C |
| ATOM | 7498 | NH1 | ARG | B | 448 | 34.909 | −24.564 | 87.135 | 1.00 | 30.34 | N |
| ATOM | 7499 | NH2 | ARG | B | 448 | 36.934 | −25.247 | 86.276 | 1.00 | 33.54 | N |
| ATOM | 7500 | C | ARG | B | 448 | 31.795 | −20.971 | 85.484 | 1.00 | 26.72 | C |
| ATOM | 7501 | O | ARG | B | 448 | 32.376 | −21.499 | 86.437 | 1.00 | 26.88 | O |
| ATOM | 7515 | N | ASN | B | 449 | 30.925 | −19.973 | 85.651 | 1.00 | 26.59 | N |
| ATOM | 7516 | CA | ASN | B | 449 | 30.643 | −19.430 | 86.996 | 1.00 | 26.57 | C |
| ATOM | 7517 | CB | ASN | B | 449 | 30.387 | −17.920 | 86.911 | 1.00 | 27.04 | C |
| ATOM | 7518 | CG | ASN | B | 449 | 31.634 | −17.118 | 87.223 | 1.00 | 28.38 | C |
| ATOM | 7519 | OD1 | ASN | B | 449 | 32.519 | −16.953 | 86.369 | 1.00 | 30.50 | O |
| ATOM | 7520 | ND2 | ASN | B | 449 | 31.739 | −16.653 | 88.467 | 1.00 | 30.31 | N |
| ATOM | 7521 | C | ASN | B | 449 | 29.514 | −20.123 | 87.774 | 1.00 | 25.66 | C |
| ATOM | 7522 | O | ASN | B | 449 | 29.040 | −19.621 | 88.811 | 1.00 | 25.95 | O |
| ATOM | 7529 | N | ASN | B | 450 | 29.133 | −21.309 | 87.311 | 1.00 | 24.51 | N |
| ATOM | 7530 | CA | ASN | B | 450 | 27.921 | −21.953 | 87.789 | 1.00 | 23.46 | C |
| ATOM | 7531 | CB | ASN | B | 450 | 27.566 | −23.112 | 86.867 | 1.00 | 23.16 | C |
| ATOM | 7532 | CG | ASN | B | 450 | 26.163 | −23.560 | 87.050 | 1.00 | 21.23 | C |
| ATOM | 7533 | OD1 | ASN | B | 450 | 25.901 | −24.444 | 87.831 | 1.00 | 18.82 | O |
| ATOM | 7534 | ND2 | ASN | B | 450 | 25.237 | −22.917 | 86.353 | 1.00 | 19.39 | N |
| ATOM | 7535 | C | ASN | B | 450 | 28.046 | −22.446 | 89.216 | 1.00 | 23.09 | C |
| ATOM | 7536 | O | ASN | B | 450 | 28.915 | −23.259 | 89.502 | 1.00 | 22.55 | O |
| ATOM | 7543 | N | LEU | B | 451 | 27.187 | −21.962 | 90.116 | 1.00 | 23.13 | N |
| ATOM | 7544 | CA | LEU | B | 451 | 27.264 | −22.420 | 91.506 | 1.00 | 23.28 | C |
| ATOM | 7545 | CB | LEU | B | 451 | 26.837 | −21.367 | 92.535 | 1.00 | 23.63 | C |
| ATOM | 7546 | CG | LEU | B | 451 | 25.457 | −20.737 | 92.564 | 1.00 | 24.59 | C |
| ATOM | 7547 | CD1 | LEU | B | 451 | 24.963 | −20.581 | 93.997 | 1.00 | 24.29 | C |
| ATOM | 7548 | CD2 | LEU | B | 451 | 25.542 | −19.373 | 91.905 | 1.00 | 27.31 | C |
| ATOM | 7549 | C | LEU | B | 451 | 26.578 | −23.724 | 91.805 | 1.00 | 22.46 | C |
| ATOM | 7550 | O | LEU | B | 451 | 26.918 | −24.358 | 92.801 | 1.00 | 22.86 | O |
| ATOM | 7562 | N | LEU | B | 452 | 25.644 | −24.158 | 90.972 | 1.00 | 21.82 | N |
| ATOM | 7563 | CA | LEU | B | 452 | 25.055 | −25.473 | 91.196 | 1.00 | 21.41 | C |
| ATOM | 7564 | CB | LEU | B | 452 | 23.912 | −25.747 | 90.232 | 1.00 | 21.14 | C |
| ATOM | 7565 | CG | LEU | B | 452 | 22.669 | −24.881 | 90.413 | 1.00 | 21.92 | C |
| ATOM | 7566 | CD1 | LEU | B | 452 | 21.509 | −25.614 | 89.784 | 1.00 | 21.80 | C |
| ATOM | 7567 | CD2 | LEU | B | 452 | 22.367 | −24.518 | 91.888 | 1.00 | 21.48 | C |
| ATOM | 7568 | C | LEU | B | 452 | 26.129 | −26.542 | 91.043 | 1.00 | 21.09 | C |
| ATOM | 7569 | O | LEU | B | 452 | 26.102 | −27.558 | 91.734 | 1.00 | 20.40 | O |
| ATOM | 7581 | N | ILE | B | 453 | 27.065 | −26.281 | 90.130 | 1.00 | 21.24 | N |
| ATOM | 7582 | CA | ILE | B | 453 | 28.201 | −27.156 | 89.856 | 1.00 | 21.38 | C |
| ATOM | 7583 | CB | ILE | B | 453 | 28.848 | −26.778 | 88.501 | 1.00 | 21.33 | C |
| ATOM | 7584 | CG1 | ILE | B | 453 | 27.872 | −27.039 | 87.348 | 1.00 | 21.30 | C |
| ATOM | 7585 | CD1 | ILE | B | 453 | 27.339 | −28.437 | 87.257 | 1.00 | 21.17 | C |
| ATOM | 7586 | CG2 | ILE | B | 453 | 30.173 | −27.505 | 88.289 | 1.00 | 21.81 | C |
| ATOM | 7587 | C | ILE | B | 453 | 29.209 | −27.097 | 90.996 | 1.00 | 21.22 | C |
| ATOM | 7588 | O | ILE | B | 453 | 29.785 | −28.112 | 91.344 | 1.00 | 20.85 | O |
| ATOM | 7600 | N | GLU | B | 454 | 29.405 | −25.924 | 91.590 | 1.00 | 21.64 | N |
| ATOM | 7601 | CA | GLU | B | 454 | 30.193 | −25.836 | 92.828 | 1.00 | 22.36 | C |
| ATOM | 7602 | CB | GLU | B | 454 | 30.256 | −24.411 | 93.385 | 1.00 | 22.90 | C |
| ATOM | 7603 | CG | GLU | B | 454 | 31.015 | −23.416 | 92.529 | 1.00 | 25.00 | C |
| ATOM | 7604 | CD | GLU | B | 454 | 32.498 | −23.455 | 92.771 | 1.00 | 28.01 | C |
| ATOM | 7605 | OE1 | GLU | B | 454 | 32.915 | −23.479 | 93.961 | 1.00 | 31.23 | O |
| ATOM | 7606 | OE2 | GLU | B | 454 | 33.248 | −23.459 | 91.764 | 1.00 | 29.63 | O |
| ATOM | 7607 | C | GLU | B | 454 | 29.568 | −26.705 | 93.894 | 1.00 | 21.83 | C |
| ATOM | 7608 | O | GLU | B | 454 | 30.261 | −27.434 | 94.580 | 1.00 | 21.68 | O |
| ATOM | 7615 | N | MET | B | 455 | 28.254 | −26.588 | 94.052 | 1.00 | 21.87 | N |
| ATOM | 7616 | CA | MET | B | 455 | 27.525 | −27.413 | 94.999 | 1.00 | 21.89 | C |
| ATOM | 7617 | CB | MET | B | 455 | 26.039 | −27.029 | 95.064 | 1.00 | 21.83 | C |
| ATOM | 7618 | CG | MET | B | 455 | 25.766 | −25.613 | 95.603 | 1.00 | 21.77 | C |
| ATOM | 7619 | SD | MET | B | 455 | 26.597 | −25.187 | 97.143 | 1.00 | 22.46 | S |
| ATOM | 7620 | CE | MET | B | 455 | 25.732 | −26.266 | 98.335 | 1.00 | 21.63 | C |
| ATOM | 7621 | C | MET | B | 455 | 27.670 | −28.883 | 94.641 | 1.00 | 22.11 | C |
| ATOM | 7622 | O | MET | B | 455 | 27.910 | −29.693 | 95.518 | 1.00 | 21.96 | O |
| ATOM | 7632 | N | LEU | B | 456 | 27.566 | −29.220 | 93.360 | 1.00 | 22.74 | N |
| ATOM | 7633 | CA | LEU | B | 456 | 27.618 | −30.616 | 92.916 | 1.00 | 23.40 | C |
| ATOM | 7634 | CB | LEU | B | 456 | 27.273 | −30.693 | 91.433 | 1.00 | 23.45 | C |
| ATOM | 7635 | CG | LEU | B | 456 | 27.045 | −32.087 | 90.842 | 1.00 | 24.35 | C |
| ATOM | 7636 | CD1 | LEU | B | 456 | 25.627 | −32.582 | 91.106 | 1.00 | 24.33 | C |
| ATOM | 7637 | CD2 | LEU | B | 456 | 27.338 | −32.091 | 89.346 | 1.00 | 24.45 | C |
| ATOM | 7638 | C | LEU | B | 456 | 28.990 | −31.250 | 93.160 | 1.00 | 24.19 | C |
| ATOM | 7639 | O | LEU | B | 456 | 29.086 | −32.421 | 93.513 | 1.00 | 23.84 | O |
| ATOM | 7651 | N | GLN | B | 457 | 30.039 | −30.448 | 93.009 | 1.00 | 25.41 | N |
| ATOM | 7652 | CA | GLN | B | 457 | 31.417 | −30.911 | 93.077 | 1.00 | 26.93 | C |
| ATOM | 7653 | CB | GLN | B | 457 | 32.258 | −30.204 | 92.015 | 1.00 | 26.95 | C |
| ATOM | 7654 | CG | GLN | B | 457 | 32.009 | −30.696 | 90.601 | 1.00 | 27.52 | C |
| ATOM | 7655 | CD | GLN | B | 457 | 33.220 | −30.482 | 89.730 | 1.00 | 28.65 | C |
| ATOM | 7656 | OE1 | GLN | B | 457 | 33.340 | −29.448 | 89.077 | 1.00 | 30.29 | O |
| ATOM | 7657 | NE2 | GLN | B | 457 | 34.145 | −31.439 | 89.745 | 1.00 | 30.23 | N |
| ATOM | 7658 | C | GLN | B | 457 | 32.042 | −30.675 | 94.456 | 1.00 | 28.35 | C |
| ATOM | 7659 | O | GLN | B | 457 | 33.252 | −30.448 | 94.579 | 1.00 | 28.63 | O |
| ATOM | 7668 | N | ALA | B | 458 | 31.207 | −30.707 | 95.485 | 1.00 | 29.95 | N |
| ATOM | 7669 | CA | ALA | B | 458 | 31.683 | −30.782 | 96.852 | 1.00 | 31.48 | C |
| ATOM | 7670 | CB | ALA | B | 458 | 30.931 | −29.774 | 97.723 | 1.00 | 31.49 | C |
| ATOM | 7671 | C | ALA | B | 458 | 31.428 | −32.221 | 97.312 | 1.00 | 32.83 | C |
| ATOM | 7672 | O | ALA | B | 458 | 30.765 | −32.989 | 96.599 | 1.00 | 33.11 | O |
| ATOM | 7678 | N | LYS | B | 459 | 31.977 | −32.571 | 98.479 | 1.00 | 34.31 | N |
| ATOM | 7679 | CA | LYS | B | 459 | 31.629 | −33.793 | 99.256 | 1.00 | 35.60 | C |
| ATOM | 7680 | CB | LYS | B | 459 | 30.359 | −34.516 | 98.736 | 1.00 | 35.65 | C |
| ATOM | 7681 | CG | LYS | B | 459 | 29.669 | −35.449 | 99.755 | 1.00 | 35.40 | C |
| ATOM | 7682 | CD | LYS | B | 459 | 30.062 | −36.930 | 99.555 | 1.00 | 36.33 | C |
| ATOM | 7683 | CE | LYS | B | 459 | 28.917 | −37.885 | 99.923 | 1.00 | 37.28 | C |
| ATOM | 7684 | NZ | LYS | B | 459 | 28.376 | −37.655 | 101.317 | 1.00 | 38.97 | N |
| ATOM | 7685 | C | LYS | B | 459 | 32.804 | −34.781 | 99.346 | 1.00 | 36.63 | C |
| ATOM | 7686 | O | LYS | B | 459 | 33.657 | −34.811 | 98.438 | 1.00 | 37.92 | O |
| ATOM | 7687 | OXT | LYS | B | 459 | 33.694 | −34.880 | 98.481 | 1.00 | 37.82 | O |
| ATOM | 7701 | N | GLU | P | 741 | 7.583 | 31.933 | 94.328 | 1.00 | 22.27 | N |
| ATOM | 7702 | CA | GLU | P | 741 | 8.478 | 30.965 | 93.616 | 1.00 | 22.83 | C |
| ATOM | 7703 | CB | GLU | P | 741 | 9.939 | 31.320 | 93.884 | 1.00 | 22.92 | C |
| ATOM | 7704 | CG | GLU | P | 741 | 10.426 | 30.946 | 95.275 | 1.00 | 24.13 | C |
| ATOM | 7705 | CD | GLU | P | 741 | 11.609 | 30.002 | 95.240 | 1.00 | 25.50 | C |
| ATOM | 7706 | OE1 | GLU | P | 741 | 11.429 | 28.757 | 95.371 | 1.00 | 25.82 | O |
| ATOM | 7707 | OE2 | GLU | P | 741 | 12.729 | 30.536 | 95.079 | 1.00 | 27.09 | O |
| ATOM | 7708 | C | GLU | P | 741 | 8.179 | 29.508 | 94.018 | 1.00 | 22.38 | C |
| ATOM | 7709 | O | GLU | P | 741 | 7.774 | 29.254 | 95.145 | 1.00 | 22.01 | O |
| ATOM | 7715 | N | ASN | P | 742 | 8.392 | 28.565 | 93.097 | 1.00 | 22.40 | N |
| ATOM | 7716 | CA | ASN | P | 742 | 7.913 | 27.181 | 93.267 | 1.00 | 22.52 | C |
| ATOM | 7717 | CB | ASN | P | 742 | 7.629 | 26.524 | 91.902 | 1.00 | 22.87 | C |
| ATOM | 7718 | CG | ASN | P | 742 | 6.694 | 25.302 | 91.998 | 1.00 | 22.97 | C |
| ATOM | 7719 | OD1 | ASN | P | 742 | 6.702 | 24.557 | 92.978 | 1.00 | 23.50 | O |
| ATOM | 7720 | ND2 | ASN | P | 742 | 5.871 | 25.116 | 90.966 | 1.00 | 23.35 | N |
| ATOM | 7721 | C | ASN | P | 742 | 8.895 | 26.339 | 94.052 | 1.00 | 22.21 | C |
| ATOM | 7722 | O | ASN | P | 742 | 9.969 | 26.021 | 93.556 | 1.00 | 21.92 | O |
| ATOM | 7729 | N | ALA | P | 743 | 8.510 | 25.982 | 95.276 | 1.00 | 22.04 | N |
| ATOM | 7730 | CA | ALA | P | 743 | 9.395 | 25.272 | 96.192 | 1.00 | 21.92 | C |
| ATOM | 7731 | CB | ALA | P | 743 | 8.795 | 25.219 | 97.591 | 1.00 | 21.54 | C |
| ATOM | 7732 | C | ALA | P | 743 | 9.668 | 23.881 | 95.685 | 1.00 | 22.04 | C |
| ATOM | 7733 | O | ALA | P | 743 | 10.739 | 23.360 | 95.886 | 1.00 | 22.41 | O |
| ATOM | 7739 | N | LEU | P | 744 | 8.692 | 23.297 | 95.011 | 1.00 | 22.75 | N |
| ATOM | 7740 | CA | LEU | P | 744 | 8.828 | 21.973 | 94.421 | 1.00 | 23.19 | C |
| ATOM | 7741 | CB | LEU | P | 744 | 7.463 | 21.455 | 93.936 | 1.00 | 23.29 | C |
| ATOM | 7742 | CG | LEU | P | 744 | 7.386 | 20.069 | 93.284 | 1.00 | 22.36 | C |
| ATOM | 7743 | CD1 | LEU | P | 744 | 7.749 | 18.975 | 94.243 | 1.00 | 20.47 | C |
| ATOM | 7744 | CD2 | LEU | P | 744 | 5.998 | 19.827 | 92.701 | 1.00 | 23.32 | C |
| ATOM | 7745 | C | LEU | P | 744 | 9.819 | 21.958 | 93.278 | 1.00 | 23.49 | C |
| ATOM | 7746 | O | LEU | P | 744 | 10.649 | 21.069 | 93.193 | 1.00 | 24.84 | O |
| ATOM | 7758 | N | LEU | P | 745 | 9.729 | 22.927 | 92.384 | 1.00 | 23.13 | N |
| ATOM | 7759 | CA | LEU | P | 745 | 10.666 | 23.010 | 91.286 | 1.00 | 22.64 | C |
| ATOM | 7760 | CB | LEU | P | 745 | 10.291 | 24.093 | 90.274 | 1.00 | 22.23 | C |
| ATOM | 7761 | CG | LEU | P | 745 | 9.109 | 23.848 | 89.355 | 1.00 | 21.35 | C |
| ATOM | 7762 | CD1 | LEU | P | 745 | 8.913 | 25.045 | 88.488 | 1.00 | 19.96 | C |
| ATOM | 7763 | CD2 | LEU | P | 745 | 9.325 | 22.600 | 88.496 | 1.00 | 22.35 | C |
| ATOM | 7764 | C | LEU | P | 745 | 12.061 | 23.257 | 91.791 | 1.00 | 22.33 | C |
| ATOM | 7765 | O | LEU | P | 745 | 12.982 | 22.653 | 91.294 | 1.00 | 23.00 | O |
| ATOM | 7777 | N | ARG | P | 746 | 12.243 | 24.132 | 92.768 | 1.00 | 21.95 | N |
| ATOM | 7778 | CA | ARG | P | 746 | 13.588 | 24.317 | 93.310 | 1.00 | 21.74 | C |
| ATOM | 7779 | CB | ARG | P | 746 | 13.640 | 25.397 | 94.393 | 1.00 | 22.00 | C |
| ATOM | 7780 | CG | ARG | P | 746 | 15.082 | 25.748 | 94.785 | 1.00 | 23.05 | C |
| ATOM | 7781 | CD | ARG | P | 746 | 15.255 | 26.771 | 95.872 | 1.00 | 24.65 | C |
| ATOM | 7782 | NE | ARG | P | 746 | 16.072 | 27.907 | 95.421 | 1.00 | 26.68 | N |
| ATOM | 7783 | CZ | ARG | P | 746 | 15.605 | 28.901 | 94.667 | 1.00 | 26.31 | C |
| ATOM | 7784 | NH1 | ARG | P | 746 | 14.346 | 28.890 | 94.263 | 1.00 | 26.42 | N |
| ATOM | 7785 | NH2 | ARG | P | 746 | 16.397 | 29.904 | 94.301 | 1.00 | 26.05 | N |
| ATOM | 7786 | C | ARG | P | 746 | 14.135 | 23.011 | 93.883 | 1.00 | 20.96 | C |
| ATOM | 7787 | O | ARG | P | 746 | 15.311 | 22.688 | 93.716 | 1.00 | 20.34 | O |
| ATOM | 7801 | N | TYR | P | 747 | 13.271 | 22.271 | 94.562 | 1.00 | 20.56 | N |
| ATOM | 7802 | CA | TYR | P | 747 | 13.671 | 21.021 | 95.191 | 1.00 | 20.31 | C |
| ATOM | 7803 | CB | TYR | P | 747 | 12.539 | 20.489 | 96.088 | 1.00 | 19.69 | C |
| ATOM | 7804 | CG | TYR | P | 747 | 12.758 | 19.080 | 96.618 | 1.00 | 16.87 | C |
| ATOM | 7805 | CD1 | TYR | P | 747 | 13.858 | 18.760 | 97.400 | 1.00 | 13.85 | C |
| ATOM | 7806 | CE1 | TYR | P | 747 | 14.052 | 17.469 | 97.870 | 1.00 | 11.93 | C |
| ATOM | 7807 | CZ | TYR | P | 747 | 13.141 | 16.492 | 97.564 | 1.00 | 11.67 | C |
| ATOM | 7808 | OH | TYR | P | 747 | 13.284 | 15.208 | 98.001 | 1.00 | 9.56 | O |
| ATOM | 7809 | CE2 | TYR | P | 747 | 12.045 | 16.789 | 96.793 | 1.00 | 13.94 | C |
| ATOM | 7810 | CD2 | TYR | P | 747 | 11.857 | 18.071 | 96.324 | 1.00 | 15.63 | C |
| ATOM | 7811 | C | TYR | P | 747 | 14.102 | 19.975 | 94.150 | 1.00 | 20.33 | C |
| ATOM | 7812 | O | TYR | P | 747 | 15.086 | 19.298 | 94.327 | 1.00 | 19.89 | O |
| ATOM | 7822 | N | LEU | P | 748 | 13.359 | 19.874 | 93.057 | 1.00 | 20.86 | N |
| ATOM | 7823 | CA | LEU | P | 748 | 13.593 | 18.836 | 92.078 | 1.00 | 20.79 | C |
| ATOM | 7824 | CB | LEU | P | 748 | 12.361 | 18.662 | 91.215 | 1.00 | 20.83 | C |
| ATOM | 7825 | CG | LEU | P | 748 | 11.096 | 18.248 | 91.956 | 1.00 | 21.06 | C |
| ATOM | 7826 | CD1 | LEU | P | 748 | 9.961 | 18.231 | 90.935 | 1.00 | 21.29 | C |
| ATOM | 7827 | CD2 | LEU | P | 748 | 11.250 | 16.876 | 92.645 | 1.00 | 20.53 | C |
| ATOM | 7828 | C | LEU | P | 748 | 14.801 | 19.155 | 91.214 | 1.00 | 21.05 | C |
| ATOM | 7829 | O | LEU | P | 748 | 15.431 | 18.261 | 90.663 | 1.00 | 19.63 | O |
| ATOM | 7841 | N | LEU | P | 749 | 15.109 | 20.443 | 91.131 | 1.00 | 21.63 | N |
| ATOM | 7842 | CA | LEU | P | 749 | 16.232 | 20.933 | 90.377 | 1.00 | 22.48 | C |
| ATOM | 7843 | CB | LEU | P | 749 | 15.933 | 22.342 | 89.857 | 1.00 | 22.82 | C |
| ATOM | 7844 | CG | LEU | P | 749 | 14.927 | 22.499 | 88.709 | 1.00 | 21.55 | C |
| ATOM | 7845 | CD1 | LEU | P | 749 | 14.506 | 23.954 | 88.626 | 1.00 | 21.81 | C |
| ATOM | 7846 | CD2 | LEU | P | 749 | 15.527 | 22.048 | 87.381 | 1.00 | 20.78 | C |
| ATOM | 7847 | C | LEU | P | 749 | 17.501 | 20.969 | 91.213 | 1.00 | 23.25 | C |
| ATOM | 7848 | O | LEU | P | 749 | 18.578 | 20.833 | 90.669 | 1.00 | 23.26 | O |
| ATOM | 7860 | N | ASP | P | 750 | 17.371 | 21.159 | 92.525 | 1.00 | 24.72 | N |
| ATOM | 7861 | CA | ASP | P | 750 | 18.534 | 21.318 | 93.413 | 1.00 | 25.72 | C |
| ATOM | 7862 | CB | ASP | P | 750 | 18.211 | 22.199 | 94.633 | 1.00 | 26.16 | C |
| ATOM | 7863 | CG | ASP | P | 750 | 18.238 | 23.715 | 94.305 | 1.00 | 27.16 | C |
| ATOM | 7864 | OD1 | ASP | P | 750 | 18.566 | 24.110 | 93.156 | 1.00 | 28.19 | O |
| ATOM | 7865 | OD2 | ASP | P | 750 | 17.944 | 24.588 | 95.148 | 1.00 | 28.34 | O |
| ATOM | 7866 | C | ASP | P | 750 | 19.058 | 19.946 | 93.826 | 1.00 | 26.28 | C |
| ATOM | 7867 | O | ASP | P | 750 | 20.218 | 19.604 | 93.554 | 1.00 | 26.58 | O |
| ATOM | 7872 | N | LYS | P | 751 | 18.217 | 19.137 | 94.446 | 1.00 | 26.38 | N |
| ATOM | 7873 | CA | LYS | P | 751 | 18.450 | 17.698 | 94.345 | 1.00 | 27.07 | C |
| ATOM | 7874 | CB | LYS | P | 751 | 19.556 | 17.203 | 95.286 | 1.00 | 27.10 | C |
| ATOM | 7875 | CG | LYS | P | 751 | 19.799 | 15.668 | 95.285 | 1.00 | 26.43 | C |
| ATOM | 7876 | CD | LYS | P | 751 | 20.136 | 15.110 | 93.903 | 1.00 | 25.89 | C |
| ATOM | 7877 | CE | LYS | P | 751 | 19.840 | 13.621 | 93.804 | 1.00 | 25.20 | C |
| ATOM | 7878 | NZ | LYS | P | 751 | 18.467 | 13.367 | 93.275 | 1.00 | 24.62 | N |
| ATOM | 7879 | C | LYS | P | 751 | 17.163 | 16.919 | 94.534 | 1.00 | 27.56 | C |
| ATOM | 7880 | O | LYS | P | 751 | 16.379 | 17.102 | 95.457 | 1.00 | 27.49 | O |
| ATOM | 7881 | OXT | LYS | P | 751 | 16.887 | 16.067 | 93.692 | 1.00 | 28.85 | O |
| ATOM | 7895 | N | ASP | P | 752 | 14.845 | 15.866 | 93.845 | 1.00 | 35.80 | N |
| ATOM | 7896 | CA | ASP | P | 752 | 14.670 | 14.462 | 94.206 | 1.00 | 36.05 | C |
| ATOM | 7897 | CB | ASP | P | 752 | 15.191 | 14.228 | 95.637 | 1.00 | 35.76 | C |
| ATOM | 7898 | CG | ASP | P | 752 | 15.232 | 12.751 | 96.043 | 1.00 | 35.84 | C |
| ATOM | 7899 | OD1 | ASP | P | 752 | 14.176 | 12.200 | 96.431 | 1.00 | 36.42 | O |
| ATOM | 7900 | OD2 | ASP | P | 752 | 16.282 | 12.070 | 96.062 | 1.00 | 34.75 | O |
| ATOM | 7901 | C | ASP | P | 752 | 13.171 | 14.134 | 94.079 | 1.00 | 36.56 | C |
| ATOM | 7902 | O | ASP | P | 752 | 12.785 | 13.311 | 93.197 | 1.00 | 37.05 | O |
| ATOM | 7906 | N | GLU | Q | 741 | 38.045 | −24.398 | 99.950 | 1.00 | 25.99 | N |
| ATOM | 7907 | CA | GLU | Q | 741 | 37.135 | −25.566 | 99.745 | 1.00 | 25.78 | C |
| ATOM | 7908 | CB | GLU | Q | 741 | 36.917 | −26.330 | 101.063 | 1.00 | 25.87 | C |
| ATOM | 7909 | CG | GLU | Q | 741 | 36.705 | −25.451 | 102.299 | 1.00 | 26.45 | C |
| ATOM | 7910 | CD | GLU | Q | 741 | 35.331 | −25.623 | 102.946 | 1.00 | 27.21 | C |
| ATOM | 7911 | OE1 | GLU | Q | 741 | 35.264 | −26.213 | 104.052 | 1.00 | 27.93 | O |
| ATOM | 7912 | OE2 | GLU | Q | 741 | 34.319 | −25.162 | 102.361 | 1.00 | 27.00 | O |
| ATOM | 7913 | C | GLU | Q | 741 | 35.790 | −25.165 | 99.118 | 1.00 | 25.63 | C |
| ATOM | 7914 | O | GLU | Q | 741 | 35.438 | −25.677 | 98.054 | 1.00 | 25.74 | O |
| ATOM | 7920 | N | ASN | Q | 742 | 35.045 | −24.262 | 99.766 | 1.00 | 25.32 | N |
| ATOM | 7921 | CA | ASN | Q | 742 | 33.708 | −23.855 | 99.284 | 1.00 | 25.13 | C |
| ATOM | 7922 | CB | ASN | Q | 742 | 32.607 | −24.798 | 99.821 | 1.00 | 25.21 | C |
| ATOM | 7923 | CG | ASN | Q | 742 | 31.309 | −24.741 | 98.987 | 1.00 | 27.06 | C |
| ATOM | 7924 | OD1 | ASN | Q | 742 | 31.028 | −23.747 | 98.298 | 1.00 | 29.42 | O |
| ATOM | 7925 | ND2 | ASN | Q | 742 | 30.517 | −25.813 | 99.047 | 1.00 | 29.32 | N |
| ATOM | 7926 | C | ASN | Q | 742 | 33.404 | −22.377 | 99.616 | 1.00 | 24.48 | C |
| ATOM | 7927 | O | ASN | Q | 742 | 32.407 | −22.058 | 100.270 | 1.00 | 23.87 | O |
| ATOM | 7934 | N | ALA | Q | 743 | 34.271 | −21.490 | 99.116 | 1.00 | 23.91 | N |
| ATOM | 7935 | CA | ALA | Q | 743 | 34.260 | −20.060 | 99.459 | 1.00 | 23.32 | C |
| ATOM | 7936 | CB | ALA | Q | 743 | 35.622 | −19.409 | 99.095 | 1.00 | 23.24 | C |
| ATOM | 7937 | C | ALA | Q | 743 | 33.101 | −19.257 | 98.846 | 1.00 | 22.56 | C |
| ATOM | 7938 | O | ALA | Q | 743 | 32.793 | −18.164 | 99.316 | 1.00 | 22.52 | O |
| ATOM | 7944 | N | LEU | Q | 744 | 32.465 | −19.779 | 97.803 | 1.00 | 21.98 | N |
| ATOM | 7945 | CA | LEU | Q | 744 | 31.279 | −19.120 | 97.243 | 1.00 | 21.60 | C |
| ATOM | 7946 | CB | LEU | Q | 744 | 30.949 | −19.669 | 95.846 | 1.00 | 21.85 | C |
| ATOM | 7947 | CG | LEU | Q | 744 | 29.983 | −18.892 | 94.916 | 1.00 | 23.13 | C |
| ATOM | 7948 | CD1 | LEU | Q | 744 | 29.721 | −17.437 | 95.364 | 1.00 | 23.26 | C |
| ATOM | 7949 | CD2 | LEU | Q | 744 | 30.463 | −18.925 | 93.429 | 1.00 | 23.57 | C |
| ATOM | 7950 | C | LEU | Q | 744 | 30.080 | −19.273 | 98.186 | 1.00 | 20.71 | C |
| ATOM | 7951 | O | LEU | Q | 744 | 29.380 | −18.303 | 98.461 | 1.00 | 20.33 | O |
| ATOM | 7963 | N | LEU | Q | 745 | 29.869 | −20.484 | 98.691 | 1.00 | 19.83 | N |
| ATOM | 7964 | CA | LEU | Q | 745 | 28.816 | −20.743 | 99.683 | 1.00 | 19.67 | C |
| ATOM | 7965 | CB | LEU | Q | 745 | 28.708 | −22.249 | 99.948 | 1.00 | 19.59 | C |
| ATOM | 7966 | CG | LEU | Q | 745 | 27.380 | −22.895 | 100.363 | 1.00 | 19.72 | C |
| ATOM | 7967 | CD1 | LEU | Q | 745 | 27.643 | −24.069 | 101.299 | 1.00 | 19.23 | C |
| ATOM | 7968 | CD2 | LEU | Q | 745 | 26.372 | −21.934 | 100.977 | 1.00 | 19.90 | C |
| ATOM | 7969 | C | LEU | Q | 745 | 29.057 | −20.011 | 101.013 | 1.00 | 19.51 | C |
| ATOM | 7970 | O | LEU | Q | 745 | 28.121 | −19.537 | 101.656 | 1.00 | 18.85 | O |
| ATOM | 7982 | N | ARG | Q | 746 | 30.327 | −19.942 | 101.413 | 1.00 | 19.67 | N |
| ATOM | 7983 | CA | ARG | Q | 746 | 30.746 | −19.241 | 102.627 | 1.00 | 19.59 | C |
| ATOM | 7984 | CB | ARG | Q | 746 | 32.254 | −19.411 | 102.857 | 1.00 | 19.83 | C |
| ATOM | 7985 | CG | ARG | Q | 746 | 32.684 | −19.212 | 104.302 | 1.00 | 20.54 | C |
| ATOM | 7986 | CD | ARG | Q | 746 | 34.138 | −19.572 | 104.574 | 1.00 | 21.57 | C |
| ATOM | 7987 | NE | ARG | Q | 746 | 34.276 | −20.619 | 105.602 | 1.00 | 21.15 | N |
| ATOM | 7988 | CZ | ARG | Q | 746 | 34.544 | −21.909 | 105.369 | 1.00 | 20.65 | C |
| ATOM | 7989 | NH1 | ARG | Q | 746 | 34.704 | −22.384 | 104.131 | 1.00 | 20.65 | N |
| ATOM | 7990 | NH2 | ARG | Q | 746 | 34.647 | −22.741 | 106.394 | 1.00 | 20.71 | N |
| ATOM | 7991 | C | ARG | Q | 746 | 30.406 | −17.762 | 102.566 | 1.00 | 19.23 | C |
| ATOM | 7992 | O | ARG | Q | 746 | 29.868 | −17.212 | 103.518 | 1.00 | 19.25 | O |
| ATOM | 8006 | N | TYR | Q | 747 | 30.713 | −17.129 | 101.442 | 1.00 | 19.07 | N |
| ATOM | 8007 | CA | TYR | Q | 747 | 30.428 | −15.714 | 101.254 | 1.00 | 19.43 | C |
| ATOM | 8008 | CB | TYR | Q | 747 | 30.884 | −15.263 | 99.862 | 1.00 | 19.40 | C |
| ATOM | 8009 | CG | TYR | Q | 747 | 30.492 | −13.834 | 99.561 | 1.00 | 20.10 | C |
| ATOM | 8010 | CD1 | TYR | Q | 747 | 31.145 | −12.776 | 100.200 | 1.00 | 21.02 | C |
| ATOM | 8011 | CE1 | TYR | Q | 747 | 30.800 | −11.456 | 99.954 | 1.00 | 20.26 | C |
| ATOM | 8012 | CZ | TYR | Q | 747 | 29.780 | −11.174 | 99.072 | 1.00 | 20.07 | C |
| ATOM | 8013 | OH | TYR | Q | 747 | 29.460 | −9.860 | 98.856 | 1.00 | 19.67 | O |
| ATOM | 8014 | CE2 | TYR | Q | 747 | 29.089 | −12.206 | 98.423 | 1.00 | 20.56 | C |
| ATOM | 8015 | CD2 | TYR | Q | 747 | 29.450 | −13.531 | 98.672 | 1.00 | 20.17 | C |
| ATOM | 8016 | C | TYR | Q | 747 | 28.939 | −15.429 | 101.435 | 1.00 | 19.55 | C |
| ATOM | 8017 | O | TYR | Q | 747 | 28.542 | −14.456 | 102.075 | 1.00 | 19.32 | O |
| ATOM | 8027 | N | LEU | Q | 748 | 28.133 | −16.308 | 100.851 | 1.00 | 20.30 | N |
| ATOM | 8028 | CA | LEU | Q | 748 | 26.675 | −16.235 | 100.884 | 1.00 | 20.81 | C |
| ATOM | 8029 | CB | LEU | Q | 748 | 26.076 | −17.406 | 100.074 | 1.00 | 20.89 | C |
| ATOM | 8030 | CG | LEU | Q | 748 | 25.648 | −17.246 | 98.597 | 1.00 | 21.19 | C |
| ATOM | 8031 | CD1 | LEU | Q | 748 | 26.554 | −16.394 | 97.776 | 1.00 | 21.30 | C |
| ATOM | 8032 | CD2 | LEU | Q | 748 | 25.513 | −18.620 | 97.927 | 1.00 | 21.92 | C |
| ATOM | 8033 | C | LEU | Q | 748 | 26.121 | −16.243 | 102.317 | 1.00 | 21.05 | C |
| ATOM | 8034 | O | LEU | Q | 748 | 25.164 | −15.540 | 102.620 | 1.00 | 21.22 | O |
| ATOM | 8046 | N | LEU | Q | 749 | 26.732 | −17.030 | 103.192 | 1.00 | 21.60 | N |
| ATOM | 8047 | CA | LEU | Q | 749 | 26.287 | −17.134 | 104.585 | 1.00 | 22.24 | C |
| ATOM | 8048 | CB | LEU | Q | 749 | 26.724 | −18.481 | 105.153 | 1.00 | 22.05 | C |
| ATOM | 8049 | CG | LEU | Q | 749 | 26.048 | −19.682 | 104.488 | 1.00 | 21.89 | C |
| ATOM | 8050 | CD1 | LEU | Q | 749 | 26.846 | −20.949 | 104.723 | 1.00 | 21.72 | C |
| ATOM | 8051 | CD2 | LEU | Q | 749 | 24.632 | −19.838 | 105.017 | 1.00 | 21.90 | C |
| ATOM | 8052 | C | LEU | Q | 749 | 26.771 | −15.999 | 105.512 | 1.00 | 22.98 | C |
| ATOM | 8053 | O | LEU | Q | 749 | 26.154 | −15.721 | 106.544 | 1.00 | 23.04 | O |
| ATOM | 8065 | N | ASP | Q | 750 | 27.858 | −15.333 | 105.138 | 1.00 | 23.89 | N |
| ATOM | 8066 | CA | ASP | Q | 750 | 28.469 | −14.324 | 106.002 | 1.00 | 24.77 | C |
| ATOM | 8067 | CB | ASP | Q | 750 | 29.979 | −14.185 | 105.688 | 1.00 | 24.95 | C |
| ATOM | 8068 | CG | ASP | Q | 750 | 30.838 | −15.210 | 106.429 | 1.00 | 25.43 | C |
| ATOM | 8069 | OD1 | ASP | Q | 750 | 30.280 | −16.188 | 106.996 | 1.00 | 26.05 | O |
| ATOM | 8070 | OD2 | ASP | Q | 750 | 32.084 | −15.114 | 106.488 | 1.00 | 25.58 | O |
| ATOM | 8071 | C | ASP | Q | 750 | 27.798 | −12.959 | 105.907 | 1.00 | 25.17 | C |
| ATOM | 8072 | O | ASP | Q | 750 | 28.300 | −12.005 | 106.478 | 1.00 | 25.49 | O |
| ATOM | 8077 | N | LYS | Q | 751 | 26.668 | −12.869 | 105.217 | 1.00 | 25.94 | N |
| ATOM | 8078 | CA | LYS | Q | 751 | 26.010 | −11.590 | 104.948 | 1.00 | 26.70 | C |
| ATOM | 8079 | CB | LYS | Q | 751 | 24.570 | −11.589 | 105.485 | 1.00 | 26.72 | C |
| ATOM | 8080 | CG | LYS | Q | 751 | 23.671 | −10.502 | 104.856 | 1.00 | 26.43 | C |
| ATOM | 8081 | CD | LYS | Q | 751 | 23.726 | −9.156 | 105.624 | 1.00 | 26.00 | C |
| ATOM | 8082 | CE | LYS | Q | 751 | 23.156 | −9.277 | 107.040 | 1.00 | 25.72 | C |
| ATOM | 8083 | NZ | LYS | Q | 751 | 24.042 | −8.735 | 108.112 | 1.00 | 25.70 | N |
| ATOM | 8084 | C | LYS | Q | 751 | 26.789 | −10.389 | 105.490 | 1.00 | 27.21 | C |
| ATOM | 8085 | O | LYS | Q | 751 | 27.723 | −9.909 | 104.844 | 1.00 | 28.04 | O |
| ATOM | 8086 | OXT | LYS | Q | 751 | 26.561 | −9.833 | 106.576 | 1.00 | 27.92 | O |
| ATOM | 8100 | O30 | LIG | L | 1 | −11.719 | 5.990 | 83.371 | 1.00 | 19.90 | O |
| ATOM | 8101 | C30 | LIG | L | 1 | −10.759 | 5.840 | 84.111 | 1.00 | 21.00 | C |
| ATOM | 8102 | C31 | LIG | L | 1 | −9.377 | 5.322 | 83.689 | 1.00 | 21.65 | C |
| ATOM | 8103 | C32 | LIG | L | 1 | −8.502 | 6.522 | 83.304 | 1.00 | 22.04 | C |
| ATOM | 8104 | C33 | LIG | L | 1 | −6.988 | 6.306 | 83.257 | 1.00 | 23.71 | C |
| ATOM | 8105 | C34 | LIG | L | 1 | −6.310 | 7.488 | 82.530 | 1.00 | 24.65 | C |
| ATOM | 8106 | C35 | LIG | L | 1 | −4.876 | 7.836 | 82.977 | 1.00 | 24.95 | C |
| ATOM | 8107 | C36 | LIG | L | 1 | −4.183 | 8.860 | 82.059 | 1.00 | 24.27 | C |
| ATOM | 8108 | C37 | LIG | L | 1 | −2.858 | 9.326 | 82.686 | 1.00 | 25.39 | C |
| ATOM | 8109 | C38 | LIG | L | 1 | −1.882 | 10.020 | 81.726 | 1.00 | 24.22 | C |
| ATOM | 8110 | C39 | LIG | L | 1 | −0.815 | 10.753 | 82.528 | 1.00 | 23.94 | C |
| ATOM | 8111 | C40 | LIG | L | 1 | −0.204 | 11.922 | 81.782 | 1.00 | 24.72 | C |
| ATOM | 8112 | C41 | LIG | L | 1 | −1.053 | 13.197 | 81.645 | 1.00 | 24.94 | C |
| ATOM | 8113 | C42 | LIG | L | 1 | −0.757 | 14.203 | 82.744 | 1.00 | 24.72 | C |
| ATOM | 8114 | C43 | LIG | L | 1 | −1.207 | 15.593 | 82.371 | 1.00 | 25.68 | C |
| ATOM | 8115 | C44 | LIG | L | 1 | −1.057 | 16.556 | 83.289 | 1.00 | 27.06 | C |
| ATOM | 8116 | C45 | LIG | L | 1 | −1.459 | 18.004 | 83.072 | 1.00 | 28.47 | C |
| ATOM | 8117 | O4 | LIG | L | 1 | −10.953 | 6.216 | 85.486 | 1.00 | 21.73 | O |
| ATOM | 8118 | C1 | LIG | L | 1 | −11.942 | 7.145 | 85.912 | 1.00 | 21.64 | C |
| ATOM | 8119 | C6 | LIG | L | 1 | −11.356 | 7.940 | 87.074 | 1.00 | 21.28 | C |
| ATOM | 8120 | C5 | LIG | L | 1 | −12.442 | 8.804 | 87.667 | 1.00 | 20.25 | C |
| ATOM | 8121 | O14 | LIG | L | 1 | −12.814 | 9.692 | 86.637 | 1.00 | 20.59 | O |
| ATOM | 8122 | P1 | LIG | L | 1 | −13.186 | 11.198 | 86.946 | 1.00 | 20.27 | P |
| ATOM | 8123 | O12 | LIG | L | 1 | −13.815 | 11.704 | 85.695 | 1.00 | 20.89 | O |
| ATOM | 8124 | O13 | LIG | L | 1 | −11.976 | 11.849 | 87.565 | 1.00 | 21.92 | O |
| ATOM | 8125 | O11 | LIG | L | 1 | −14.315 | 11.130 | 88.079 | 1.00 | 22.86 | O |
| ATOM | 8126 | C3 | LIG | L | 1 | −15.594 | 10.495 | 87.925 | 1.00 | 23.76 | C |
| ATOM | 8127 | C2 | LIG | L | 1 | −16.251 | 10.361 | 89.290 | 1.00 | 25.14 | C |
| ATOM | 8128 | N1 | LIG | L | 1 | −16.706 | 11.388 | 89.880 | 1.00 | 26.74 | N |
| ATOM | 8129 | O7 | LIG | L | 1 | −10.341 | 8.793 | 86.562 | 1.00 | 21.42 | O |
| ATOM | 8130 | C8 | LIG | L | 1 | −8.967 | 8.455 | 86.820 | 1.00 | 20.71 | C |
| ATOM | 8131 | O10 | LIG | L | 1 | −8.667 | 7.496 | 87.521 | 1.00 | 22.43 | O |
| ATOM | 8132 | C11 | LIG | L | 1 | −7.950 | 9.359 | 86.188 | 1.00 | 21.34 | C |
| ATOM | 8133 | C12 | LIG | L | 1 | −6.793 | 9.562 | 87.140 | 1.00 | 22.63 | C |
| ATOM | 8134 | C13 | LIG | L | 1 | −5.731 | 10.441 | 86.506 | 1.00 | 24.25 | C |
| ATOM | 8135 | C14 | LIG | L | 1 | −4.892 | 11.007 | 87.627 | 1.00 | 24.96 | C |
| ATOM | 8136 | C15 | LIG | L | 1 | −3.658 | 11.753 | 87.182 | 1.00 | 25.07 | C |
| ATOM | 8137 | C16 | LIG | L | 1 | −3.992 | 13.177 | 86.847 | 1.00 | 26.47 | C |
| ATOM | 8138 | C17 | LIG | L | 1 | −3.607 | 13.412 | 85.401 | 1.00 | 26.01 | C |
| ATOM | 8139 | C18 | LIG | L | 1 | −4.223 | 14.691 | 84.828 | 1.00 | 25.09 | C |
| ATOM | 8140 | C19 | LIG | L | 1 | −5.068 | 14.626 | 83.685 | 1.00 | 25.18 | C |
| ATOM | 8141 | C20 | LIG | L | 1 | −5.349 | 13.247 | 83.119 | 1.00 | 25.99 | C |
| ATOM | 8142 | C21 | LIG | L | 1 | −5.951 | 13.287 | 81.737 | 1.00 | 26.69 | C |
| ATOM | 8143 | C22 | LIG | L | 1 | −4.898 | 13.037 | 80.700 | 1.00 | 26.38 | C |
| ATOM | 8144 | C23 | LIG | L | 1 | −5.505 | 12.537 | 79.406 | 1.00 | 27.17 | C |
| ATOM | 8145 | C24 | LIG | L | 1 | −4.493 | 12.818 | 78.329 | 1.00 | 27.54 | C |
| ATOM | 8146 | C25 | LIG | L | 1 | −4.425 | 14.297 | 77.966 | 1.00 | 28.07 | C |
| ATOM | 8147 | O30 | LIG | L | 2 | 12.865 | −28.246 | 76.567 | 1.00 | 22.60 | O |
| ATOM | 8148 | C30 | LIG | L | 2 | 13.249 | −27.091 | 76.564 | 1.00 | 20.95 | C |
| ATOM | 8149 | C31 | LIG | L | 2 | 12.369 | −25.951 | 76.971 | 1.00 | 19.52 | C |
| ATOM | 8150 | C32 | LIG | L | 2 | 12.941 | −25.325 | 78.236 | 1.00 | 19.87 | C |
| ATOM | 8151 | C33 | LIG | L | 2 | 12.866 | −26.265 | 79.437 | 1.00 | 20.50 | C |
| ATOM | 8152 | C34 | LIG | L | 2 | 13.219 | −25.522 | 80.709 | 1.00 | 21.93 | C |
| ATOM | 8153 | C35 | LIG | L | 2 | 13.165 | −26.404 | 81.953 | 1.00 | 23.06 | C |
| ATOM | 8154 | C36 | LIG | L | 2 | 13.436 | −25.497 | 83.158 | 1.00 | 24.57 | C |
| ATOM | 8155 | C37 | LIG | L | 2 | 13.404 | −26.201 | 84.516 | 1.00 | 24.64 | C |
| ATOM | 8156 | C38 | LIG | L | 2 | 14.295 | −25.465 | 85.509 | 1.00 | 25.45 | C |
| ATOM | 8157 | C39 | LIG | L | 2 | 13.748 | −25.553 | 86.943 | 1.00 | 27.56 | C |
| ATOM | 8158 | C40 | LIG | L | 2 | 14.700 | −24.900 | 87.959 | 1.00 | 26.53 | C |
| ATOM | 8159 | C41 | LIG | L | 2 | 14.848 | −25.648 | 89.286 | 1.00 | 27.39 | C |
| ATOM | 8160 | C42 | LIG | L | 2 | 15.478 | −27.032 | 89.170 | 1.00 | 26.94 | C |
| ATOM | 8161 | C43 | LIG | L | 2 | 16.867 | −27.143 | 89.754 | 1.00 | 24.66 | C |
| ATOM | 8162 | C44 | LIG | L | 2 | 17.144 | −28.317 | 90.331 | 1.00 | 25.04 | C |
| ATOM | 8163 | C45 | LIG | L | 2 | 18.479 | −28.648 | 90.970 | 1.00 | 26.26 | C |
| ATOM | 8164 | O4 | LIG | L | 2 | 14.600 | −26.827 | 76.168 | 1.00 | 20.74 | O |
| ATOM | 8165 | C1 | LIG | L | 2 | 15.525 | −27.882 | 76.002 | 1.00 | 22.20 | C |
| ATOM | 8166 | C6 | LIG | L | 2 | 16.793 | −27.428 | 76.686 | 1.00 | 22.13 | C |
| ATOM | 8167 | C5 | LIG | L | 2 | 17.935 | −28.406 | 76.388 | 1.00 | 22.38 | C |
| ATOM | 8168 | O14 | LIG | L | 2 | 17.593 | −29.755 | 76.719 | 1.00 | 21.10 | O |
| ATOM | 8169 | P1 | LIG | L | 2 | 18.734 | −30.792 | 77.099 | 1.00 | 19.19 | P |
| ATOM | 8170 | O12 | LIG | L | 2 | 18.149 | −32.154 | 77.332 | 1.00 | 19.54 | O |
| ATOM | 8171 | O13 | LIG | L | 2 | 19.600 | −30.138 | 78.128 | 1.00 | 21.51 | O |
| ATOM | 8172 | O11 | LIG | L | 2 | 19.591 | −30.853 | 75.760 | 1.00 | 22.08 | O |
| ATOM | 8173 | C3 | LIG | L | 2 | 18.984 | −31.205 | 74.513 | 1.00 | 23.80 | C |
| ATOM | 8174 | C2 | LIG | L | 2 | 19.844 | −30.734 | 73.378 | 1.00 | 23.47 | C |
| ATOM | 8175 | N1 | LIG | L | 2 | 20.461 | −31.595 | 72.698 | 1.00 | 24.47 | N |
| ATOM | 8176 | O7 | LIG | L | 2 | 16.431 | −27.385 | 78.060 | 1.00 | 24.25 | O |
| ATOM | 8177 | C8 | LIG | L | 2 | 17.211 | −26.542 | 78.917 | 1.00 | 23.54 | C |
| ATOM | 8178 | O10 | LIG | L | 2 | 17.946 | −25.736 | 78.410 | 1.00 | 23.62 | O |
| ATOM | 8179 | C11 | LIG | L | 2 | 17.051 | −26.712 | 80.405 | 1.00 | 25.02 | C |
| ATOM | 8180 | C12 | LIG | L | 2 | 18.040 | −25.862 | 81.184 | 1.00 | 24.97 | C |
| ATOM | 8181 | C13 | LIG | L | 2 | 17.811 | −25.994 | 82.680 | 1.00 | 24.63 | C |
| ATOM | 8182 | C14 | LIG | L | 2 | 18.968 | −25.304 | 83.386 | 1.00 | 25.31 | C |
| ATOM | 8183 | C15 | LIG | L | 2 | 18.789 | −25.296 | 84.909 | 1.00 | 26.01 | C |
| ATOM | 8184 | C16 | LIG | L | 2 | 19.618 | −26.369 | 85.610 | 1.00 | 25.86 | C |
| ATOM | 8185 | C17 | LIG | L | 2 | 18.766 | −27.155 | 86.600 | 1.00 | 26.05 | C |
| ATOM | 8186 | C18 | LIG | L | 2 | 19.083 | −28.652 | 86.717 | 1.00 | 25.23 | C |
| ATOM | 8187 | C19 | LIG | L | 2 | 18.078 | −29.612 | 86.430 | 1.00 | 23.96 | C |
| ATOM | 8188 | C20 | LIG | L | 2 | 16.738 | −29.098 | 85.971 | 1.00 | 25.75 | C |
| ATOM | 8189 | C21 | LIG | L | 2 | 15.647 | −30.143 | 86.017 | 1.00 | 27.27 | C |
| ATOM | 8190 | C22 | LIG | L | 2 | 14.377 | −29.553 | 86.596 | 1.00 | 27.53 | C |
| ATOM | 8191 | C23 | LIG | L | 2 | 13.151 | −30.396 | 86.274 | 1.00 | 28.74 | C |
| ATOM | 8192 | C24 | LIG | L | 2 | 12.201 | −30.494 | 87.453 | 1.00 | 29.15 | C |
| ATOM | 8193 | C25 | LIG | L | 2 | 12.751 | −31.301 | 88.599 | 1.00 | 29.35 | C |
| ATOM | 8194 | O | HOH | S | 1 | −11.848 | 0.045 | 73.744 | 1.00 | 6.41 | O |
| ATOM | 8195 | O | HOH | S | 2 | 21.576 | 28.395 | 89.940 | 1.00 | 28.18 | O |
| ATOM | 8196 | O | HOH | S | 3 | −2.496 | −4.035 | 81.610 | 1.00 | 12.19 | O |
| ATOM | 8197 | O | HOH | S | 4 | −12.110 | 18.170 | 79.275 | 1.00 | 21.34 | O |
| ATOM | 8198 | O | HOH | S | 5 | 14.541 | 34.073 | 66.873 | 1.00 | 30.52 | O |
| ATOM | 8199 | O | HOH | S | 6 | −3.878 | 17.940 | 96.398 | 1.00 | 20.59 | O |
| ATOM | 8200 | O | HOH | S | 7 | −11.366 | 24.254 | 77.896 | 1.00 | 31.35 | O |
| ATOM | 8201 | O | HOH | S | 8 | 8.608 | 10.615 | 95.038 | 1.00 | 25.93 | O |
| ATOM | 8202 | O | HOH | S | 9 | −6.633 | 16.553 | 69.383 | 1.00 | 24.92 | O |
| ATOM | 8203 | O | HOH | S | 10 | 6.518 | −26.501 | 71.593 | 1.00 | 31.61 | O |
| ATOM | 8204 | O | HOH | S | 11 | 17.529 | 22.711 | 56.913 | 1.00 | 20.73 | O |
| ATOM | 8205 | O | HOH | S | 12 | 6.497 | 30.502 | 83.315 | 1.00 | 24.76 | O |
| ATOM | 8206 | O | HOH | S | 13 | 6.423 | 9.243 | 76.156 | 1.00 | 21.99 | O |
| ATOM | 8207 | O | HOH | S | 14 | −13.750 | 4.877 | 88.801 | 1.00 | 20.07 | O |
| ATOM | 8208 | O | HOH | S | 15 | 13.687 | 40.062 | 80.451 | 1.00 | 18.74 | O |
| ATOM | 8209 | O | HOH | S | 16 | 7.918 | 5.797 | 78.272 | 1.00 | 30.80 | O |
| ATOM | 8210 | O | HOH | S | 17 | 11.093 | 5.849 | 65.735 | 1.00 | 24.49 | O |
| ATOM | 8211 | O | HOH | S | 18 | 0.335 | 7.306 | 79.139 | 1.00 | 19.00 | O |
| ATOM | 8212 | O | HOH | S | 19 | 5.772 | 32.988 | 76.726 | 1.00 | 29.37 | O |
| ATOM | 8213 | O | HOH | S | 20 | 21.341 | 37.526 | 78.216 | 1.00 | 24.14 | O |
| ATOM | 8214 | O | HOH | S | 21 | 25.119 | 14.785 | 62.332 | 1.00 | 25.59 | O |
| ATOM | 8215 | O | HOH | S | 22 | 1.070 | 26.163 | 64.252 | 1.00 | 29.08 | O |
| ATOM | 8216 | O | HOH | S | 23 | −5.577 | 9.663 | 75.298 | 1.00 | 19.04 | O |
| ATOM | 8217 | O | HOH | S | 24 | 17.283 | 24.160 | 58.969 | 1.00 | 22.51 | O |
| ATOM | 8218 | O | HOH | S | 25 | 10.486 | 1.320 | 79.206 | 1.00 | 37.34 | O |
| ATOM | 8219 | O | HOH | S | 26 | 7.855 | 14.982 | 77.472 | 1.00 | 23.64 | O |
| ATOM | 8220 | O | HOH | S | 27 | −8.160 | −1.021 | 68.907 | 1.00 | 23.10 | O |
| ATOM | 8221 | O | HOH | S | 28 | 24.046 | 19.142 | 62.678 | 1.00 | 31.48 | O |
| ATOM | 8222 | O | HOH | S | 29 | −14.326 | 14.259 | 84.635 | 1.00 | 19.68 | O |
| ATOM | 8223 | O | HOH | S | 30 | −8.102 | 6.987 | 67.685 | 1.00 | 22.49 | O |
| ATOM | 8224 | O | HOH | S | 31 | 26.692 | 15.403 | 64.996 | 1.00 | 26.67 | O |
| ATOM | 8225 | O | HOH | S | 32 | −7.898 | 5.568 | 70.330 | 1.00 | 28.68 | O |
| ATOM | 8226 | O | HOH | S | 33 | −1.085 | 19.111 | 96.380 | 1.00 | 21.49 | O |
| ATOM | 8227 | O | HOH | S | 34 | 3.380 | 24.556 | 75.151 | 1.00 | 43.72 | O |
| ATOM | 8228 | O | HOH | S | 35 | 27.739 | 27.252 | 82.058 | 1.00 | 38.98 | O |
| ATOM | 8229 | O | HOH | S | 36 | 19.112 | 15.192 | 58.685 | 1.00 | 19.63 | O |
| ATOM | 8230 | O | HOH | S | 37 | −2.911 | 26.134 | 71.807 | 1.00 | 28.60 | O |
| ATOM | 8231 | O | HOH | S | 38 | 7.559 | 0.608 | 89.004 | 1.00 | 25.81 | O |
| ATOM | 8232 | O | HOH | S | 39 | −11.195 | 19.801 | 81.326 | 1.00 | 22.02 | O |
| ATOM | 8233 | O | HOH | S | 40 | −0.690 | 13.278 | 89.901 | 1.00 | 27.83 | O |
| ATOM | 8234 | O | HOH | S | 41 | −9.569 | 16.425 | 65.308 | 1.00 | 35.44 | O |
| ATOM | 8235 | O | HOH | S | 42 | −2.051 | 15.092 | 60.530 | 1.00 | 33.27 | O |
| ATOM | 8236 | O | HOH | S | 43 | −8.203 | 13.529 | 99.160 | 1.00 | 32.28 | O |
| ATOM | 8237 | O | HOH | S | 44 | −4.814 | 8.626 | 97.478 | 1.00 | 25.22 | O |
| ATOM | 8238 | O | HOH | S | 45 | 7.913 | 39.628 | 72.687 | 1.00 | 34.13 | O |
| ATOM | 8239 | O | HOH | S | 46 | −5.692 | 7.101 | 68.121 | 1.00 | 23.77 | O |
| ATOM | 8240 | O | HOH | S | 47 | 3.350 | 4.337 | 90.405 | 1.00 | 29.34 | O |
| ATOM | 8241 | O | HOH | S | 48 | −0.647 | 24.199 | 79.409 | 1.00 | 42.40 | O |
| ATOM | 8242 | O | HOH | S | 49 | 2.430 | 9.235 | 83.252 | 1.00 | 24.84 | O |
| ATOM | 8243 | O | HOH | S | 50 | 1.833 | 25.905 | 82.808 | 1.00 | 35.93 | O |
| ATOM | 8244 | O | HOH | S | 51 | 4.202 | 9.699 | 93.766 | 1.00 | 31.21 | O |
| ATOM | 8245 | O | HOH | S | 52 | 24.929 | 33.687 | 78.248 | 1.00 | 33.27 | O |
| ATOM | 8246 | O | HOH | S | 53 | −12.909 | 13.827 | 70.633 | 1.00 | 29.29 | O |
| ATOM | 8247 | O | HOH | S | 54 | 1.099 | 6.980 | 81.982 | 1.00 | 16.07 | O |
| ATOM | 8248 | O | HOH | S | 55 | 18.076 | 14.482 | 86.944 | 1.00 | 25.16 | O |
| ATOM | 8249 | O | HOH | S | 56 | 15.837 | 13.743 | 74.988 | 1.00 | 14.80 | O |
| ATOM | 8250 | O | HOH | S | 57 | −6.914 | −2.601 | 72.504 | 1.00 | 24.99 | O |
| ATOM | 8251 | O | HOH | S | 58 | 10.407 | 12.014 | 59.007 | 1.00 | 22.51 | O |
| ATOM | 8252 | O | HOH | S | 59 | −10.634 | 9.895 | 69.885 | 1.00 | 27.70 | O |
| ATOM | 8253 | O | HOH | S | 60 | 24.600 | 22.845 | 77.427 | 1.00 | 19.23 | O |
| ATOM | 8254 | O | HOH | S | 61 | −5.381 | 23.053 | 68.143 | 1.00 | 20.02 | O |
| ATOM | 8255 | O | HOH | S | 62 | −7.874 | 17.568 | 96.874 | 1.00 | 16.75 | O |
| ATOM | 8256 | O | HOH | S | 63 | −14.724 | 18.040 | 91.770 | 1.00 | 24.86 | O |
| ATOM | 8257 | O | HOH | S | 64 | 3.772 | 25.380 | 67.441 | 1.00 | 20.01 | O |
| ATOM | 8258 | O | HOH | S | 65 | −13.116 | 15.350 | 72.940 | 1.00 | 30.75 | O |
| ATOM | 8259 | O | HOH | S | 66 | −10.109 | 6.778 | 63.300 | 1.00 | 28.25 | O |
| ATOM | 8260 | O | HOH | S | 67 | 3.285 | 11.943 | 94.844 | 1.00 | 24.18 | O |
| ATOM | 8261 | O | HOH | S | 68 | 12.154 | 6.604 | 61.460 | 1.00 | 20.68 | O |
| ATOM | 8262 | O | HOH | S | 69 | 7.496 | 6.724 | 75.616 | 1.00 | 32.39 | O |
| ATOM | 8263 | O | HOH | S | 70 | 10.233 | 9.332 | 88.642 | 1.00 | 29.72 | O |
| ATOM | 8264 | O | HOH | S | 71 | −9.454 | −1.504 | 80.672 | 1.00 | 10.90 | O |
| ATOM | 8265 | O | HOH | S | 72 | 20.166 | 23.032 | 64.629 | 1.00 | 24.63 | O |
| ATOM | 8266 | O | HOH | S | 73 | −18.791 | 7.731 | 82.550 | 1.00 | 17.95 | O |
| ATOM | 8267 | O | HOH | S | 74 | 2.666 | 11.033 | 80.786 | 1.00 | 23.16 | O |
| ATOM | 8268 | O | HOH | S | 75 | −18.404 | 2.018 | 77.603 | 1.00 | 6.45 | O |
| ATOM | 8269 | O | HOH | S | 76 | −7.109 | 13.236 | 64.292 | 1.00 | 22.10 | O |
| ATOM | 8270 | O | HOH | S | 77 | 8.180 | 26.356 | 67.308 | 1.00 | 25.74 | O |
| ATOM | 8271 | O | HOH | S | 78 | 3.053 | 22.573 | 73.022 | 1.00 | 36.70 | O |
| ATOM | 8272 | O | HOH | S | 79 | −7.584 | 24.463 | 75.954 | 1.00 | 25.35 | O |
| ATOM | 8273 | O | HOH | S | 80 | 22.147 | 7.393 | 76.175 | 1.00 | 30.54 | O |
| ATOM | 8274 | O | HOH | S | 81 | 8.766 | 12.582 | 77.945 | 1.00 | 25.36 | O |
| ATOM | 8275 | O | HOH | S | 82 | 7.722 | 28.541 | 89.456 | 1.00 | 34.45 | O |
| ATOM | 8276 | O | HOH | S | 83 | 19.692 | 32.076 | 83.533 | 1.00 | 19.66 | O |
| ATOM | 8277 | O | HOH | S | 84 | 23.730 | 16.044 | 81.113 | 1.00 | 23.60 | O |
| ATOM | 8278 | O | HOH | S | 85 | 11.361 | −1.523 | 83.138 | 1.00 | 33.59 | O |
| ATOM | 8279 | O | HOH | S | 86 | 11.020 | 26.643 | 71.517 | 1.00 | 17.48 | O |
| ATOM | 8280 | O | HOH | S | 87 | 13.993 | 11.203 | 83.138 | 1.00 | 22.45 | O |
| ATOM | 8281 | O | HOH | S | 88 | −0.647 | 11.959 | 93.928 | 1.00 | 20.40 | O |
| ATOM | 8282 | O | HOH | S | 89 | −6.158 | 19.109 | 95.982 | 1.00 | 15.52 | O |
| ATOM | 8283 | O | HOH | S | 90 | 10.105 | 4.971 | 70.240 | 1.00 | 32.18 | O |
| ATOM | 8284 | O | HOH | S | 91 | −3.240 | 4.402 | 72.816 | 1.00 | 26.21 | O |
| ATOM | 8285 | O | HOH | S | 92 | −14.385 | −1.695 | 79.207 | 1.00 | 9.75 | O |
| ATOM | 8286 | O | HOH | S | 93 | −2.498 | 4.193 | 69.718 | 1.00 | 18.69 | O |
| ATOM | 8287 | O | HOH | S | 94 | 8.795 | 26.670 | 78.625 | 1.00 | 13.34 | O |
| ATOM | 8288 | O | HOH | S | 95 | 2.256 | 24.010 | 81.688 | 1.00 | 20.63 | O |
| ATOM | 8289 | O | HOH | S | 96 | 12.188 | 26.263 | 68.871 | 1.00 | 20.08 | O |
| ATOM | 8290 | O | HOH | S | 97 | 9.827 | −21.901 | 117.485 | 1.00 | 22.08 | O |
| ATOM | 8291 | O | HOH | S | 98 | 24.936 | −38.085 | 84.324 | 1.00 | 7.67 | O |
| ATOM | 8292 | O | HOH | S | 99 | 0.643 | −27.106 | 87.354 | 1.00 | 19.89 | O |
| ATOM | 8293 | O | HOH | S | 100 | 10.132 | −23.404 | 88.290 | 1.00 | 17.09 | O |
| ATOM | 8294 | O | HOH | S | 101 | 12.180 | −21.693 | 87.878 | 1.00 | 15.75 | O |
| ATOM | 8295 | O | HOH | S | 102 | 14.536 | −32.296 | 109.447 | 1.00 | 19.31 | O |
| ATOM | 8296 | O | HOH | S | 103 | 4.032 | −36.034 | 84.112 | 1.00 | 19.63 | O |
| ATOM | 8297 | O | HOH | S | 104 | 25.344 | −19.909 | 85.942 | 1.00 | 20.10 | O |
| ATOM | 8298 | O | HOH | S | 105 | 8.107 | −30.337 | 86.204 | 1.00 | 18.04 | O |
| ATOM | 8299 | O | HOH | S | 106 | 13.407 | −23.569 | 91.389 | 1.00 | 20.83 | O |
| ATOM | 8300 | O | HOH | S | 107 | −7.362 | −25.880 | 113.607 | 1.00 | 20.66 | O |
| ATOM | 8301 | O | HOH | S | 108 | 23.600 | −33.313 | 97.913 | 1.00 | 25.74 | O |
| ATOM | 8302 | O | HOH | S | 109 | 1.602 | −28.712 | 76.418 | 1.00 | 16.79 | O |
| ATOM | 8303 | O | HOH | S | 110 | 21.274 | −31.951 | 97.554 | 1.00 | 23.33 | O |
| ATOM | 8304 | O | HOH | S | 111 | 9.761 | −20.076 | 105.094 | 1.00 | 18.09 | O |
| ATOM | 8305 | O | HOH | S | 112 | 14.144 | −21.859 | 89.703 | 1.00 | 24.29 | O |
| ATOM | 8306 | O | HOH | S | 113 | 8.228 | −22.274 | 95.106 | 1.00 | 20.83 | O |
| ATOM | 8307 | O | HOH | S | 114 | −1.733 | −35.448 | 109.541 | 1.00 | 19.44 | O |
| ATOM | 8308 | O | HOH | S | 115 | 0.950 | −31.639 | 87.039 | 1.00 | 29.68 | O |
| ATOM | 8309 | O | HOH | S | 116 | 3.016 | −27.124 | 85.974 | 1.00 | 18.53 | O |
| ATOM | 8310 | O | HOH | S | 117 | 1.188 | −20.360 | 98.505 | 1.00 | 24.05 | O |
| ATOM | 8311 | O | HOH | S | 118 | 20.678 | −30.912 | 105.042 | 1.00 | 19.24 | O |
| ATOM | 8312 | O | HOH | S | 119 | 21.881 | −15.407 | 100.051 | 1.00 | 24.46 | O |
| ATOM | 8313 | O | HOH | S | 120 | 20.398 | −41.513 | 93.566 | 1.00 | 27.05 | O |
| ATOM | 8314 | O | HOH | S | 121 | 26.862 | −33.196 | 77.865 | 1.00 | 28.47 | O |
| ATOM | 8315 | O | HOH | S | 122 | 8.471 | −20.256 | 77.121 | 1.00 | 18.54 | O |
| ATOM | 8316 | O | HOH | S | 123 | 3.212 | −23.199 | 118.642 | 1.00 | 15.93 | O |
| ATOM | 8317 | O | HOH | S | 124 | 0.344 | −19.356 | 86.925 | 1.00 | 32.96 | O |
| ATOM | 8318 | O | HOH | S | 125 | 6.529 | −20.257 | 94.347 | 1.00 | 32.96 | O |
| ATOM | 8319 | O | HOH | S | 126 | 9.771 | −39.430 | 84.144 | 1.00 | 29.29 | O |
| ATOM | 8320 | O | HOH | S | 127 | 9.644 | −5.104 | 96.772 | 1.00 | 31.77 | O |
| ATOM | 8321 | O | HOH | S | 128 | −5.490 | −23.914 | 88.430 | 1.00 | 30.08 | O |
| ATOM | 8322 | O | HOH | S | 129 | 30.742 | −27.366 | 81.823 | 1.00 | 16.78 | O |
| ATOM | 8323 | O | HOH | S | 130 | −4.096 | −33.781 | 91.498 | 1.00 | 22.19 | O |
| ATOM | 8324 | O | HOH | S | 131 | 10.256 | −12.592 | 102.662 | 1.00 | 29.97 | O |
| ATOM | 8325 | O | HOH | S | 132 | 1.678 | −32.162 | 83.900 | 1.00 | 25.75 | O |
| ATOM | 8326 | O | HOH | S | 133 | 1.027 | −38.289 | 90.469 | 1.00 | 27.43 | O |
| ATOM | 8327 | O | HOH | S | 134 | 19.461 | −36.807 | 77.186 | 1.00 | 23.03 | O |
| ATOM | 8328 | O | HOH | S | 135 | 10.293 | −34.855 | 109.783 | 1.00 | 32.29 | O |
| ATOM | 8329 | O | HOH | S | 136 | −2.291 | −26.507 | 114.519 | 1.00 | 24.94 | O |
| ATOM | 8330 | O | HOH | S | 137 | 6.934 | −27.525 | 117.115 | 1.00 | 19.61 | O |
| ATOM | 8331 | O | HOH | S | 138 | 6.502 | −14.639 | 101.725 | 1.00 | 25.27 | O |
| ATOM | 8332 | O | HOH | S | 139 | 13.860 | −18.446 | 97.192 | 1.00 | 35.04 | O |
| ATOM | 8333 | O | HOH | S | 140 | −4.133 | −34.900 | 109.273 | 1.00 | 20.34 | O |
| ATOM | 8334 | O | HOH | S | 141 | −5.112 | −39.092 | 104.886 | 1.00 | 24.25 | O |
| ATOM | 8335 | O | HOH | S | 142 | 12.552 | −21.978 | 117.503 | 1.00 | 22.22 | O |
| ATOM | 8336 | O | HOH | S | 143 | 18.402 | −34.335 | 78.672 | 1.00 | 19.40 | O |
| ATOM | 8337 | O | HOH | S | 144 | −0.680 | −24.384 | 98.333 | 1.00 | 25.65 | O |
| ATOM | 8338 | O | HOH | S | 145 | 12.625 | −39.013 | 77.626 | 1.00 | 28.34 | O |
| ATOM | 8339 | O | HOH | S | 146 | 23.449 | −32.596 | 76.198 | 1.00 | 26.56 | O |
| ATOM | 8340 | O | HOH | S | 147 | 29.807 | −26.312 | 112.933 | 1.00 | 20.01 | O |
| ATOM | 8341 | O | HOH | S | 148 | 27.464 | −25.196 | 115.005 | 1.00 | 9.28 | O |
| ATOM | 8342 | O | HOH | S | 149 | 29.431 | −29.639 | 110.502 | 1.00 | 13.94 | O |
| ATOM | 8343 | O | HOH | S | 150 | 4.221 | −13.079 | 95.584 | 1.00 | 42.41 | O |
| ATOM | 8344 | O | HOH | S | 151 | −1.468 | −38.162 | 111.032 | 1.00 | 23.14 | O |
| ATOM | 8345 | O | HOH | S | 152 | 15.434 | −14.452 | 99.287 | 1.00 | 38.95 | O |
| ATOM | 8346 | O | HOH | S | 153 | 29.033 | −20.734 | 75.583 | 1.00 | 27.52 | O |
| ATOM | 8347 | O | HOH | S | 154 | 11.741 | −22.156 | 98.104 | 1.00 | 25.70 | O |
| ATOM | 8348 | O | HOH | S | 155 | 16.481 | −13.784 | 111.145 | 1.00 | 24.65 | O |
| ATOM | 8349 | O | HOH | S | 156 | 11.911 | −19.101 | 95.524 | 1.00 | 28.66 | O |
| ATOM | 8350 | O | HOH | S | 157 | −0.223 | −23.159 | 95.736 | 1.00 | 32.23 | O |
| ATOM | 8351 | O | HOH | S | 158 | 18.416 | −14.199 | 93.943 | 1.00 | 26.35 | O |
| ATOM | 8352 | O | HOH | S | 159 | −2.647 | −27.525 | 96.727 | 1.00 | 31.45 | O |
| ATOM | 8353 | O | HOH | S | 160 | 17.051 | −40.632 | 96.780 | 1.00 | 31.25 | O |
| ATOM | 8354 | O | HOH | S | 161 | 11.471 | −11.535 | 99.068 | 1.00 | 29.83 | O |
| ATOM | 8355 | O | HOH | S | 162 | 24.932 | −17.040 | 88.452 | 1.00 | 22.29 | O |
| ATOM | 8356 | O | HOH | S | 163 | 27.184 | −23.674 | 117.108 | 1.00 | 24.41 | O |
| ATOM | 8357 | O | HOH | S | 164 | 23.078 | −16.700 | 77.901 | 1.00 | 22.70 | O |
| ATOM | 8358 | O | HOH | S | 165 | −2.643 | −21.416 | 103.436 | 1.00 | 32.18 | O |
| ATOM | 8359 | O | HOH | S | 166 | 5.768 | −36.309 | 76.898 | 1.00 | 26.30 | O |
| ATOM | 8360 | O | HOH | S | 167 | 9.935 | −17.735 | 95.615 | 1.00 | 44.63 | O |
| ATOM | 8361 | O | HOH | S | 168 | −0.262 | −25.700 | 117.449 | 1.00 | 21.14 | O |
| ATOM | 8362 | O | HOH | S | 169 | 12.179 | −35.090 | 74.455 | 1.00 | 28.62 | O |
| ATOM | 8363 | O | HOH | S | 170 | 16.543 | −10.871 | 103.291 | 1.00 | 20.50 | O |
| ATOM | 8364 | O | HOH | S | 171 | 16.641 | −37.012 | 101.670 | 1.00 | 34.66 | O |
| ATOM | 8365 | O | HOH | S | 172 | −0.773 | −29.882 | 87.429 | 1.00 | 23.92 | O |
| ATOM | 8366 | O | HOH | S | 173 | −2.179 | −22.054 | 100.847 | 1.00 | 21.00 | O |
| ATOM | 8367 | O | HOH | S | 174 | 4.053 | −16.182 | 104.427 | 1.00 | 50.25 | O |
| ATOM | 8368 | O | HOH | S | 175 | 10.766 | −12.156 | 105.410 | 1.00 | 19.60 | O |
| ATOM | 8369 | O | HOH | S | 176 | 16.889 | −19.664 | 117.027 | 1.00 | 23.06 | O |
| ATOM | 8370 | O | HOH | S | 177 | −0.689 | −24.536 | 79.431 | 1.00 | 25.78 | O |
| ATOM | 8371 | O | HOH | S | 178 | −5.326 | −28.870 | 96.514 | 1.00 | 33.20 | O |
| ATOM | 8372 | O | HOH | S | 179 | 4.908 | −15.704 | 103.038 | 1.00 | 47.77 | O |
| ATOM | 8373 | O | HOH | S | 180 | 4.993 | −15.341 | 97.968 | 1.00 | 30.21 | O |
| ATOM | 8374 | O | HOH | S | 181 | 31.405 | −33.192 | 112.823 | 1.00 | 34.15 | O |
| ATOM | 8375 | O | HOH | S | 182 | 24.408 | −30.477 | 72.907 | 1.00 | 25.60 | O |
| ATOM | 8376 | O | HOH | S | 183 | 14.020 | −14.569 | 103.616 | 1.00 | 22.92 | O |
| ATOM | 8377 | O | HOH | S | 184 | 21.258 | −15.129 | 116.030 | 1.00 | 13.23 | O |
| ATOM | 8378 | O | HOH | S | 185 | 26.478 | −32.419 | 103.620 | 1.00 | 24.83 | O |
| ATOM | 8379 | O | HOH | S | 186 | 10.460 | −38.043 | 104.325 | 1.00 | 26.05 | O |
| ATOM | 8380 | O | HOH | S | 187 | −5.404 | −26.834 | 101.195 | 1.00 | 25.30 | O |
| ATOM | 8381 | O | HOH | S | 188 | 18.956 | −37.230 | 84.815 | 1.00 | 25.97 | O |
| ATOM | 8382 | O | HOH | S | 189 | −1.319 | −37.828 | 96.344 | 1.00 | 27.71 | O |
| ATOM | 8383 | O | HOH | S | 190 | −4.388 | −35.434 | 102.281 | 1.00 | 26.23 | O |
| ATOM | 8384 | O | HOH | S | 191 | 7.118 | −14.775 | 115.570 | 1.00 | 18.18 | O |
| ATOM | 8385 | O | HOH | S | 192 | 1.261 | −21.092 | 76.141 | 1.00 | 29.68 | O |
| ATOM | 8386 | O | HOH | S | 193 | 12.356 | −38.022 | 75.284 | 1.00 | 26.81 | O |
| ATOM | 8387 | O | HOH | S | 194 | 6.301 | 3.453 | 78.830 | 1.00 | 35.93 | O |
| ATOM | 8388 | O | HOH | S | 195 | −16.503 | 16.080 | 84.965 | 1.00 | 25.88 | O |
| ATOM | 8389 | O | HOH | S | 196 | −1.221 | −21.095 | 95.587 | 1.00 | 25.85 | O |
| ATOM | 8390 | O | HOH | S | 197 | 3.995 | −27.947 | 117.147 | 1.00 | 20.63 | O |
| ATOM | 8391 | O | HOH | S | 198 | 1.906 | −36.042 | 85.629 | 1.00 | 20.49 | O |
| ATOM | 8392 | O | HOH | S | 199 | 30.633 | −7.979 | 97.903 | 1.00 | 12.48 | O |
| ATOM | 8393 | O | HOH | S | 200 | −3.672 | −23.554 | 99.216 | 1.00 | 31.50 | O |
| ATOM | 8394 | O | HOH | S | 201 | 34.085 | −36.889 | 86.839 | 1.00 | 15.05 | O |
| ATOM | 8395 | O | HOH | S | 202 | 9.304 | 35.547 | 84.371 | 1.00 | 25.53 | O |
| ATOM | 8396 | O | HOH | S | 203 | 4.831 | −33.963 | 77.745 | 1.00 | 25.01 | O |
| ATOM | 8397 | O | HOH | S | 204 | 3.361 | −34.091 | 82.648 | 1.00 | 19.63 | O |
| ATOM | 8398 | O | HOH | S | 205 | 18.977 | −37.610 | 74.409 | 1.00 | 25.39 | O |
| ATOM | 8399 | O | HOH | S | 206 | −11.991 | 23.416 | 86.183 | 1.00 | 12.11 | O |
| ATOM | 8400 | O | HOH | S | 207 | −21.118 | 4.569 | 83.181 | 1.00 | 17.78 | O |
| ATOM | 8401 | O | HOH | S | 208 | 14.988 | −28.059 | 67.041 | 1.00 | 14.52 | O |
| ATOM | 8402 | O | HOH | S | 209 | 2.814 | 25.911 | 70.291 | 1.00 | 19.94 | O |
| ATOM | 8403 | O | HOH | S | 210 | −3.477 | −37.642 | 103.241 | 1.00 | 26.47 | O |
| ATOM | 8404 | O | HOH | S | 211 | −9.339 | 9.207 | 67.701 | 1.00 | 22.43 | O |
| ATOM | 8405 | O | HOH | S | 212 | 7.036 | 34.814 | 83.987 | 1.00 | 25.13 | O |
| ATOM | 8406 | O | HOH | S | 213 | −14.042 | 19.252 | 78.099 | 1.00 | 30.33 | O |
| ATOM | 8407 | O | HOH | S | 214 | 6.739 | −24.867 | 117.142 | 1.00 | 18.15 | O |
| ATOM | 8408 | O | HOH | S | 215 | 5.947 | 28.859 | 70.765 | 1.00 | 31.06 | O |
| ATOM | 8409 | O | HOH | S | 216 | −2.701 | 26.463 | 79.845 | 1.00 | 20.12 | O |
| ATOM | 8410 | O | HOH | S | 217 | 14.326 | 8.724 | 84.446 | 1.00 | 30.71 | O |
| ATOM | 8411 | O | HOH | S | 218 | −18.120 | 8.896 | 86.200 | 1.00 | 21.72 | O |
| ATOM | 8412 | O | HOH | S | 219 | −3.364 | −29.297 | 87.731 | 1.00 | 21.43 | O |
| ATOM | 8413 | O | HOH | S | 220 | 6.151 | 25.819 | 70.332 | 1.00 | 31.90 | O |
| ATOM | 8414 | O | HOH | S | 221 | 6.226 | 12.552 | 95.790 | 1.00 | 24.36 | O |
| ATOM | 8415 | O | HOH | S | 222 | −2.950 | −25.055 | 95.340 | 1.00 | 25.30 | O |
| ATOM | 8416 | O | HOH | S | 223 | 6.124 | −0.314 | 87.042 | 1.00 | 39.45 | O |
| ATOM | 8417 | O | HOH | S | 224 | 25.865 | −34.275 | 101.945 | 1.00 | 28.79 | O |
| ATOM | 8418 | O | HOH | S | 225 | 14.968 | −41.154 | 100.575 | 1.00 | 24.20 | O |
| ATOM | 8419 | O | HOH | S | 226 | 22.534 | −30.563 | 74.493 | 1.00 | 29.80 | O |
| ATOM | 8420 | O | HOH | S | 227 | 9.898 | 9.739 | 59.451 | 1.00 | 23.89 | O |
| ATOM | 8421 | O | HOH | S | 228 | −18.450 | 11.391 | 87.308 | 1.00 | 35.35 | O |
| ATOM | 8422 | O | HOH | S | 229 | 11.020 | 29.183 | 68.361 | 1.00 | 25.29 | O |
| ATOM | 8423 | O | HOH | S | 230 | 12.401 | 24.227 | 97.943 | 1.00 | 13.15 | O |
| ATOM | 8424 | O | HOH | S | 231 | 17.174 | 23.709 | 97.668 | 1.00 | 16.32 | O |
| ATOM | 8425 | O | HOH | S | 232 | 15.430 | 29.366 | 98.099 | 1.00 | 19.40 | O |
| ATOM | 8426 | O | HOH | S | 233 | 8.062 | −42.720 | 92.132 | 1.00 | 33.03 | O |
| ATOM | 8427 | O | HOH | S | 234 | −18.362 | 14.035 | 86.404 | 1.00 | 17.97 | O |
| ATOM | 8428 | O | HOH | S | 235 | −17.088 | 16.268 | 88.540 | 1.00 | 28.91 | O |

TABLE 3
Atomic coordinates for LRH crystal

| | | |
|---|---|---|
| HEADER | --- | XX-XXX-XX xxxx |

| COMPND | HUMAN LRH-1, LBD, CADIOLIPIN bouond, TWO GRIP-1 NB3 BOUND | | |
|---|---|---|---|
| REMARK | 3 | | |
| REMARK | 3 | REFINEMENT. | |
| REMARK | 3 | PROGRAM | : REFMAC 5.1.25 |
| REMARK | 3 | AUTHORS | : MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) : | 2.50 |
| REMARK | 3 | RESOLUTION RANGE LOW | (ANGSTROMS) : | 50.00 |
| REMARK | 3 | DATA CUTOFF | (SIGMA(F)) : | NONE |
| REMARK | 3 | COMPLETENESS FOR RANGE | (%) : | 99.37 |
| REMARK | 3 | NUMBER OF REFLECTIONS | : | 10899 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.24161 |
| REMARK | 3 | R VALUE | (WORKING SET) | : 0.23942 |

| REMARK | 3 | FREE R VALUE | | : 0.28129 |
|---|---|---|---|---|
| REMARK | 3 | FREE R VALUE TEST SET SIZE | (%) | : 5.2 |

| | | | |
|---|---|---|---|
| REMARK | 3 | FREE R VALUE TEST SET COUNT | : 595 |
| REMARK | 3 | | |
| REMARK | 3 | FIT IN THE HIGHEST RESOLUTION BIN. | |

| REMARK | 3 | TOTAL NUMBER OF BINS USED | | : 20 |
|---|---|---|---|---|
| REMARK | 3 | BIN RESOLUTION RANGE HIGH | | : 2.500 |
| REMARK | 3 | BIN RESOLUTION RANGE LOW | | : 2.565 |
| REMARK | 3 | REFLECTION IN BIN | (WORKING SET) | : 777 |
| REMARK | 3 | BIN R VALUE | (WORKING SET) | : 0.331 |

| | | | |
|---|---|---|---|
| REMARK | 3 | BIN FREE R VALUE SET COUNT | : 32 |
| REMARK | 3 | BIN FREE R VALUE | : 0.349 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 2169 |
| REMARK | 3 | | |
| REMARK | 3 | B VALUES. | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 32.206 |

| REMARK | 3 | OVERALL ANISOTROPIC B VALUE. | | |
|---|---|---|---|---|
| REMARK | 3 | B11 (A**2) : −1.28 | | |
| REMARK | 3 | B22 (A**2) : −2.04 | | |
| REMARK | 3 | B33 (A**2) : 3.32 | | |
| REMARK | 3 | B12 (A**2) : 0.00 | | |
| REMARK | 3 | B13 (A**2) : 0.00 | | |
| REMARK | 3 | B23 (A**2) : 0.00 | | |
| REMARK | 3 | | | |
| REMARK | 3 | ESTIMATED OVERALL COORDINATE ERROR. | | |
| REMARK | 3 | ESU BASED ON R VALUE | (A) : | 0.603 |
| REMARK | 3 | ESU BASED ON FREE R VALUE | (A) : | 0.320 |
| REMARK | 3 | ESU BASED ON MAXIMUM LIKELIHOOD | (A) : | 0.272 |
| REMARK | 3 | ESU FOR B VALUES BASED ON MAXIMUM LIKELIHOOD | (A**2) : | 12.874 |
| REMARK | 3 | | | |

| REMARK | 3 | CORRELATION COEFFICIENTS. | | | | |
|---|---|---|---|---|---|---|
| REMARK | 3 | CORRELATION COEFFICIENT FO-FC | : 0.937 | | | |
| REMARK | 3 | CORRELATION COEFFICIENT FO-FC FREE | : 0.924 | | | |
| REMARK | 3 | | | | | |
| REMARK | 3 | RMS DEVIATIONS FROM IDEAL VALUES | | COUNT | RMS | WEIGHT |
| REMARK | 3 | BOND LENGTHS REFINED ATOMS | (A): | 2167 ; | 0.008 ; | 0.022 |
| REMARK | 3 | BOND LENGTHS OTHERS | (A): | 2021 ; | 0.002 ; | 0.020 |

| | | | | | | |
|---|---|---|---|---|---|---|
| REMARK | 3 | BOND ANGLES REFINED ATOMS | (DEGREES): | 2913 ; | 1.034 ; | 1.999 |
| REMARK | 3 | BOND ANGLES OTHERS | (DEGREES): | 4722 ; | 0.722 ; | 3.000 |
| REMARK | 3 | TORSION ANGLES, PERIOD 1 | (DEGREES): | 250 ; | 5.261 ; | 5.000 |
| REMARK | 3 | CHIRAL-CENTER RESTRAINTS | (A**3): | 328 ; | 0.053 ; | 0.200 |

| | | | | | | |
|---|---|---|---|---|---|---|
| REMARK | 3 | GENERAL PLANES REFINED ATOMS | (A): | 2298 ; | 0.003 ; | 0.020 |
| REMARK | 3 | GENERAL PLANES OTHERS | (A): | 405 ; | 0.001 ; | 0.020 |
| REMARK | 3 | NON-BONDED CONTACTS REFINED ATOMS | (A): | 507 ; | 0.179 ; | 0.200 |
| REMARK | 3 | NON-BONDED CONTACTS OTHERS | (A): | 2284 ; | 0.192 ; | 0.200 |
| REMARK | 3 | NON-BONDED TORSION OTHERS | (A): | 1324 ; | 0.087 ; | 0.200 |
| REMARK | 3 | H-BOND (X...Y) REFINED ATOMS | (A): | 39 ; | 0.164 ; | 0.200 |
| REMARK | 3 | SYMMETRY VDW REFINED ATOMS | (A): | 10 ; | 0.153 ; | 0.200 |
| REMARK | 3 | SYMMETRY VDW OTHERS | (A): | 53 ; | 0.136 ; | 0.200 |
| REMARK | 3 | SYMMETRY H-BOND REFINED ATOMS | (A): | 4 ; | 0.372 ; | 0.200 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 1268 ; | 0.203 ; | 1.500 |
| REMARK | 3 | MAIN-CHAIN ANGLE REFINED ATOMS | (A**2): | 2037 ; | 0.396 ; | 2.000 |
| REMARK | 3 | SIDE-CHAIN BOND REFINED ATOMS | (A**2): | 899 ; | 0.729 ; | 3.000 |
| REMARK | 3 | SIDE-CHAIN ANGLE REFINED ATOMS | (A**2): | 876 ; | 1.213 ; | 4.500 |
| REMARK | 3 | | | | | |

| REMARK | 3 | NCS RESTRAINTS STATISTICS | | | | | |
|---|---|---|---|---|---|---|---|
| REMARK | 3 | NUMBER OF NCS GROUPS : NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS : 4 | | | | | |
| REMARK | 3 | | | | | | |
| REMARK | 3 | TLS GROUP : 1 | | | | | |
| REMARK | 3 | NUMBER OF COMPONENTS GROUP : 5 | | | | | |
| REMARK | 3 | COMPONENTS | C | SSSEQI | TO | C | SSSEQI |
| REMARK | 3 | RESIDUE RANGE : | A | 253 | | A | 284 |
| REMARK | 3 | RESIDUE RANGE : | A | 292 | | A | 492 |
| REMARK | 3 | RESIDUE RANGE : | L | 1 | | L | 1 |
| REMARK | 3 | RESIDUE RANGE : | L | 3 | | L | 3 |
| REMARK | 3 | RESIDUE RANGE : | S | 1 | | S | 26 |

| REMARK | 3 | ORIGIN FOR THE GROUP (A): 6.5570 28.9030 9.4730 | | | |
|---|---|---|---|---|---|
| REMARK | 3 | T TENSOR | | | |
| REMARK | 3 | T11: | 0.2196 T22: | 0.2025 | |
| REMARK | 3 | T33: | 0.1271 T12: | −0.1156 | |
| REMARK | 3 | T13: | 0.0150 T23: | −0.0164 | |
| REMARK | 3 | L TENSOR | | | |
| REMARK | 3 | L11: | 4.1042 L22: | 4.8604 | |
| REMARK | 3 | L33: | 3.3226 L12: | −1.0404 | |
| REMARK | 3 | L13: | −1.2019 L23: | 2.1420 | |
| REMARK | 3 | S TENSOR | | | |
| REMARK | 3 | S11: | 0.0552 S12: | 0.4310 S13: | 0.2096 |
| REMARK | 3 | S21: | −0.2874 S22: | 0.1482 S23: | 0.0435 |
| REMARK | 3 | S31: | −0.1607 S32: | 0.1279 S33: | −0.2034 |
| REMARK | 3 | | | | |

| REMARK | 3 | TLS GROUP : 2 | | | | | |
|---|---|---|---|---|---|---|---|
| REMARK | 3 | NUMBER OF COMPONENTS GROUP : 10 1 | | | | | |
| REMARK | 3 | COMPONENTS | C | SSSEQI | TO | C | SSSEQI |
| REMARK | 3 | RESIDUE RANGE : | S | 27 | | S | 30 |

| REMARK | 3 | ORIGIN FOR THE GROUP (A): 1.8830 13.9510 10.3950 | | | |
|---|---|---|---|---|---|
| REMARK | 3 | T TENSOR | | | |
| REMARK | 3 | T11: | 0.3899 T22: | 0.3846 | |
| REMARK | 3 | T33: | 0.3917 T12: | −0.0069 | |
| REMARK | 3 | T13: | −0.0080 T23: | −0.0028 | |
| REMARK | 3 | L TENSOR | | | |
| REMARK | 3 | L11: | 13.3866 L22: | 8.4652 | |
| REMARK | 3 | L33: | 2.3797 L12: | 16.5096 | |
| REMARK | 3 | L13: | 20.9657 L23: | 6.5128 | |
| REMARK | 3 | S TENSOR | | | |
| REMARK | 3 | S11: | −0.2123 S12: | 3.4380 S13: | −2.4695 |
| REMARK | 3 | S21: | 0.4630 S22: | 0.0037 S23: | 0.3701 |
| REMARK | 3 | S31: | −0.4595 S32: | 1.3261 S33: | 0.2086 |
| REMARK | 3 | | | | |

| REMARK | 3 | TLS GROUP : 3 | | | | | |
|---|---|---|---|---|---|---|---|
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | P | 741 | | P | 751 |
| REMARK | 3 | RESIDUE RANGE : | S | 31 | | S | 33 |

| REMARK | 3 | ORIGIN FOR THE GROUP (A): 25.2440 24.0090 10.7780 | | | |
|---|---|---|---|---|---|
| REMARK | 3 | T TENSOR | | | |
| REMARK | 3 | T11: | 0.3312 T22: | 0.3836 | |
| REMARK | 3 | T33: | 0.1885 T12: | −0.1248 | |
| REMARK | 3 | T13: | 0.0035 T23: | −0.2482 | |
| REMARK | 3 | L TENSOR | | | |
| REMARK | 3 | L11: | 27.0359 L22: | 12.8898 | |
| REMARK | 3 | L33: | 3.0488 L12: | −11.5308 | |
| REMARK | 3 | L13: | 6.9418 L23: | 9.7576 | |
| REMARK | 3 | S TENSOR | | | |
| REMARK | 3 | S11: | −0.1796 S12: | −0.7811 S13: | 0.0328 |
| REMARK | 3 | S21: | 0.0981 S22: | 0.6418 S23: | −0.9020 |
| REMARK | 3 | S31: | 0.3258 S32: | 0.3699 S33: | −0.4622 |
| REMARK | 3 | | | | |

| REMARK | 3 | TLS GROUP : 4 | | | | | |
|---|---|---|---|---|---|---|---|
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE: | Q | 742 | | Q | 751 |
| REMARK | 3 | RESIDUE RANGE: | S | 34 | | S | 34 |

| REMARK | 3 | ORIGIN FOR THE GROUP (A): 2.0970 14.6320 −9.7540 | | | |
|---|---|---|---|---|---|
| REMARK | 3 | T TENSOR | | | |
| REMARK | 3 | T11: | 0.3641 T22: | 0.7886 | |
| REMARK | 3 | T33: | 0.3224 T12: | 0.0231 | |
| REMARK | 3 | T13: | −0.0287 T23: | −0.3737 | |
| REMARK | 3 | L TENSOR | | | |
| REMARK | 3 | L11: | 12.2712 L22: | 36.0604 | |
| REMARK | 3 | L33: | 29.0357 L12: | 8.7367 | |
| REMARK | 3 | L13: | −1.5182 L23: | −6.3934 | |
| REMARK | 3 | S TENSOR | | | |
| REMARK | 3 | S11: | 0.2279 S12: | −0.5890 S13: | 0.3514 |
| REMARK | 3 | S21: | −0.2928 S22: | −0.1347 S23: | −0.9119 |
| REMARK | 3 | S31: | −1.2806 S32: | 0.5486 S33: | −0.0932 |
| REMARK | 3 | | | | |
| REMARK | 3 | | | | |

| REMARK | 3 | BULK SOLVENT MODELLING. | |
|---|---|---|---|
| REMARK | 3 | METHOD USED: BABINET MODEL WITH MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |
| LINK | | GLN A 284 LYS A 292 gap |

| | |
|---|---|
| CRYST1 | 61.010 67.000 78.210 90.00 90.00 90.00 P 21 21 21 0 |

| | |
|---|---|
| SCALE1 | 0.016391 0.000000 0.000000 0.00000 |
| SCALE2 | 0.000000 0.014925 0.000000 0.00000 |
| SCALE3 | 0.000000 0.000000 0.012786 0.00000 |

| | | | | | | | | | | | |
|---|---|---|---|---|---|---|---|---|---|---|---|
| ATOM | 1 | N | ALA | A | 253 | −0.028 | 51.603 | 1.317 | 1.00 | 35.04 | N |
| ATOM | 2 | CA | ALA | A | 253 | 0.140 | 51.454 | 2.791 | 1.00 | 35.02 | C |
| ATOM | 3 | CB | ALA | A | 253 | 0.597 | 50.029 | 3.128 | 1.00 | 34.99 | C |
| ATOM | 4 | C | ALA | A | 253 | 1.130 | 52.487 | 3.344 | 1.00 | 35.03 | C |
| ATOM | 5 | O | ALA | A | 253 | 1.654 | 53.326 | 2.601 | 1.00 | 35.03 | O |
| ATOM | 10 | N | SER | A | 254 | 1.347 | 52.434 | 4.659 | 1.00 | 34.96 | N |
| ATOM | 11 | CA | SER | A | 254 | 2.419 | 53.178 | 5.327 | 1.00 | 34.85 | C |
| ATOM | 12 | CB | SER | A | 254 | 1.872 | 54.385 | 6.098 | 1.00 | 34.74 | C |
| ATOM | 13 | OG | SER | A | 254 | 1.057 | 53.989 | 7.188 | 1.00 | 34.80 | O |
| ATOM | 14 | C | SER | A | 254 | 3.145 | 52.198 | 6.255 | 1.00 | 34.78 | C |
| ATOM | 15 | O | SER | A | 254 | 2.549 | 51.648 | 7.188 | 1.00 | 34.95 | O |
| ATOM | 21 | N | ILE | A | 255 | 4.426 | 51.975 | 5.977 | 1.00 | 34.55 | N |
| ATOM | 22 | CA | ILE | A | 255 | 5.174 | 50.888 | 6.584 | 1.00 | 34.41 | C |
| ATOM | 23 | CB | ILE | A | 255 | 5.728 | 49.955 | 5.479 | 1.00 | 34.48 | C |
| ATOM | 24 | CG1 | ILE | A | 255 | 4.617 | 49.530 | 4.504 | 1.00 | 34.49 | C |
| ATOM | 25 | CD1 | ILE | A | 255 | 5.127 | 49.148 | 3.123 | 1.00 | 34.63 | C |
| ATOM | 26 | CG2 | ILE | A | 255 | 6.371 | 48.716 | 6.097 | 1.00 | 34.54 | C |
| ATOM | 27 | C | ILE | A | 255 | 6.318 | 51.442 | 7.428 | 1.00 | 34.29 | C |
| ATOM | 28 | O | ILE | A | 255 | 7.280 | 51.965 | 6.875 | 1.00 | 34.19 | O |
| ATOM | 40 | N | PRO | A | 256 | 6.226 | 51.324 | 8.755 | 1.00 | 34.34 | N |
| ATOM | 41 | CA | PRO | A | 256 | 7.305 | 51.769 | 9.651 | 1.00 | 34.38 | C |
| ATOM | 42 | CB | PRO | A | 256 | 6.932 | 51.136 | 10.993 | 1.00 | 34.33 | C |
| ATOM | 43 | CG | PRO | A | 256 | 5.456 | 50.995 | 10.947 | 1.00 | 34.37 | C |
| ATOM | 44 | CD | PRO | A | 256 | 5.094 | 50.754 | 9.509 | 1.00 | 34.40 | C |
| ATOM | 45 | C | PRO | A | 256 | 8.664 | 51.263 | 9.190 | 1.00 | 34.52 | C |
| ATOM | 46 | O | PRO | A | 256 | 8.746 | 50.136 | 8.703 | 1.00 | 34.52 | O |
| ATOM | 54 | N | HIS | A | 257 | 9.706 | 52.075 | 9.344 | 1.00 | 34.67 | N |
| ATOM | 55 | CA | HIS | A | 257 | 10.997 | 51.775 | 8.725 | 1.00 | 34.84 | C |
| ATOM | 56 | CB | HIS | A | 257 | 12.056 | 52.818 | 9.089 | 1.00 | 34.94 | C |
| ATOM | 57 | CG | HIS | A | 257 | 13.344 | 52.637 | 8.348 | 1.00 | 35.70 | C |
| ATOM | 58 | ND1 | HIS | A | 257 | 14.576 | 52.855 | 8.926 | 1.00 | 36.95 | N |
| ATOM | 59 | CE1 | HIS | A | 257 | 15.526 | 52.614 | 8.039 | 1.00 | 37.08 | C |
| ATOM | 60 | NE2 | HIS | A | 257 | 14.956 | 52.239 | 6.908 | 1.00 | 36.60 | N |
| ATOM | 61 | CD2 | HIS | A | 257 | 13.592 | 52.244 | 7.075 | 1.00 | 36.39 | C |
| ATOM | 62 | C | HIS | A | 257 | 11.534 | 50.384 | 9.049 | 1.00 | 34.64 | C |
| ATOM | 63 | O | HIS | A | 257 | 12.142 | 49.752 | 8.190 | 1.00 | 34.67 | O |
| ATOM | 72 | N | LEU | A | 258 | 11.319 | 49.911 | 10.274 | 1.00 | 34.47 | N |
| ATOM | 73 | CA | LEU | A | 258 | 11.841 | 48.608 | 10.681 | 1.00 | 34.37 | C |
| ATOM | 74 | CB | LEU | A | 258 | 11.629 | 48.366 | 12.181 | 1.00 | 34.33 | C |
| ATOM | 75 | CG | LEU | A | 258 | 12.156 | 47.034 | 12.743 | 1.00 | 34.17 | C |
| ATOM | 76 | CD1 | LEU | A | 258 | 13.650 | 46.901 | 12.515 | 1.00 | 34.23 | C |
| ATOM | 77 | CD2 | LEU | A | 258 | 11.834 | 46.886 | 14.225 | 1.00 | 33.95 | C |
| ATOM | 78 | C | LEU | A | 258 | 11.205 | 47.485 | 9.863 | 1.00 | 34.51 | C |
| ATOM | 79 | O | LEU | A | 258 | 11.888 | 46.539 | 9.483 | 1.00 | 34.67 | O |
| ATOM | 91 | N | ILE | A | 259 | 9.901 | 47.595 | 9.601 | 1.00 | 34.47 | N |
| ATOM | 92 | CA | ILE | A | 259 | 9.184 | 46.624 | 8.773 | 1.00 | 34.23 | C |
| ATOM | 93 | CB | ILE | A | 259 | 7.650 | 46.875 | 8.816 | 1.00 | 34.21 | C |
| ATOM | 94 | CG1 | ILE | A | 259 | 7.126 | 46.688 | 10.245 | 1.00 | 34.13 | C |
| ATOM | 95 | CD1 | ILE | A | 259 | 5.608 | 46.774 | 10.386 | 1.00 | 34.05 | C |
| ATOM | 96 | CG2 | ILE | A | 259 | 6.910 | 45.936 | 7.838 | 1.00 | 34.09 | C |
| ATOM | 97 | C | ILE | A | 259 | 9.685 | 46.630 | 7.329 | 1.00 | 34.20 | C |
| ATOM | 98 | O | ILE | A | 259 | 9.696 | 45.590 | 6.683 | 1.00 | 34.37 | O |
| ATOM | 110 | N | LEU | A | 260 | 10.087 | 47.789 | 6.816 | 1.00 | 34.17 | N |
| ATOM | 111 | CA | LEU | A | 260 | 10.668 | 47.856 | 5.474 | 1.00 | 34.22 | C |
| ATOM | 112 | CB | LEU | A | 260 | 10.936 | 49.307 | 5.047 | 1.00 | 34.28 | C |
| ATOM | 113 | CG | LEU | A | 260 | 9.735 | 50.187 | 4.679 | 1.00 | 34.15 | C |
| ATOM | 114 | CD1 | LEU | A | 260 | 10.197 | 51.608 | 4.366 | 1.00 | 33.45 | C |
| ATOM | 115 | CD2 | LEU | A | 260 | 8.954 | 49.602 | 3.503 | 1.00 | 34.15 | C |
| ATOM | 116 | C | LEU | A | 260 | 11.962 | 47.045 | 5.404 | 1.00 | 34.19 | C |
| ATOM | 117 | O | LEU | A | 260 | 12.224 | 46.381 | 4.402 | 1.00 | 34.22 | O |
| ATOM | 129 | N | GLU | A | 261 | 12.756 | 47.087 | 6.473 | 1.00 | 34.17 | N |
| ATOM | 130 | CA | GLU | A | 261 | 14.004 | 46.326 | 6.533 | 1.00 | 34.21 | C |
| ATOM | 131 | CB | GLU | A | 261 | 14.906 | 46.805 | 7.683 | 1.00 | 34.22 | C |
| ATOM | 132 | CG | GLU | A | 261 | 15.294 | 48.283 | 7.636 | 1.00 | 34.51 | C |
| ATOM | 133 | CD | GLU | A | 261 | 16.406 | 48.614 | 6.642 | 1.00 | 35.08 | C |
| ATOM | 134 | OE1 | GLU | A | 261 | 16.696 | 47.801 | 5.738 | 1.00 | 35.35 | O |
| ATOM | 135 | OE2 | GLU | A | 261 | 16.996 | 49.712 | 6.761 | 1.00 | 35.39 | O |
| ATOM | 136 | C | GLU | A | 261 | 13.739 | 44.821 | 6.655 | 1.00 | 34.18 | C |
| ATOM | 137 | O | GLU | A | 261 | 14.434 | 44.022 | 6.030 | 1.00 | 34.27 | O |
| ATOM | 144 | N | LEU | A | 262 | 12.732 | 44.437 | 7.438 | 1.00 | 34.19 | N |
| ATOM | 145 | CA | LEU | A | 262 | 12.372 | 43.025 | 7.583 | 1.00 | 34.16 | C |
| ATOM | 146 | CB | LEU | A | 262 | 11.336 | 42.815 | 8.690 | 1.00 | 34.09 | C |
| ATOM | 147 | CG | LEU | A | 262 | 11.505 | 43.371 | 10.105 | 1.00 | 34.22 | C |
| ATOM | 148 | CD1 | LEU | A | 262 | 10.235 | 43.069 | 10.909 | 1.00 | 34.46 | C |
| ATOM | 149 | CD2 | LEU | A | 262 | 12.724 | 42.800 | 10.816 | 1.00 | 34.11 | C |
| ATOM | 150 | C | LEU | A | 262 | 11.819 | 42.448 | 6.276 | 1.00 | 34.16 | C |
| ATOM | 151 | O | LEU | A | 262 | 11.961 | 41.260 | 6.013 | 1.00 | 34.24 | O |
| ATOM | 163 | N | LEU | A | 263 | 11.186 | 43.294 | 5.468 | 1.00 | 34.28 | N |
| ATOM | 164 | CA | LEU | A | 263 | 10.620 | 42.880 | 4.186 | 1.00 | 34.29 | C |
| ATOM | 165 | CB | LEU | A | 263 | 9.709 | 43.970 | 3.622 | 1.00 | 34.21 | C |
| ATOM | 166 | CG | LEU | A | 263 | 8.296 | 44.003 | 4.195 | 1.00 | 34.12 | C |
| ATOM | 167 | CD1 | LEU | A | 263 | 7.651 | 45.353 | 3.913 | 1.00 | 34.11 | C |
| ATOM | 168 | CD2 | LEU | A | 263 | 7.449 | 42.857 | 3.631 | 1.00 | 33.80 | C |
| ATOM | 169 | C | LEU | A | 263 | 11.702 | 42.557 | 3.163 | 1.00 | 34.52 | C |
| ATOM | 170 | O | LEU | A | 263 | 11.517 | 41.673 | 2.326 | 1.00 | 34.65 | O |
| ATOM | 182 | N | LYS | A | 264 | 12.824 | 43.274 | 3.232 | 1.00 | 34.77 | N |
| ATOM | 183 | CA | LYS | A | 264 | 13.944 | 43.069 | 2.309 | 1.00 | 34.88 | C |
| ATOM | 184 | CB | LYS | A | 264 | 14.989 | 44.181 | 2.476 | 1.00 | 34.88 | C |
| ATOM | 185 | CG | LYS | A | 264 | 14.546 | 45.536 | 1.916 | 1.00 | 34.69 | C |
| ATOM | 186 | CD | LYS | A | 264 | 15.605 | 46.623 | 2.104 | 1.00 | 34.14 | C |
| ATOM | 187 | CE | LYS | A | 264 | 15.012 | 48.029 | 1.932 | 1.00 | 34.29 | C |
| ATOM | 188 | NZ | LYS | A | 264 | 15.905 | 48.961 | 1.171 | 1.00 | 34.46 | N |
| ATOM | 189 | C | LYS | A | 264 | 14.609 | 41.693 | 2.461 | 1.00 | 35.16 | C |
| ATOM | 190 | O | LYS | A | 264 | 15.318 | 41.244 | 1.556 | 1.00 | 35.37 | O |
| ATOM | 204 | N | CYS | A | 265 | 14.362 | 41.029 | 3.592 | 1.00 | 35.36 | N |
| ATOM | 205 | CA | CYS | A | 265 | 14.913 | 39.701 | 3.883 | 1.00 | 35.51 | C |
| ATOM | 206 | CB | CYS | A | 265 | 15.025 | 39.509 | 5.400 | 1.00 | 35.53 | C |
| ATOM | 207 | SG | CYS | A | 265 | 15.969 | 40.801 | 6.253 | 1.00 | 36.50 | S |
| ATOM | 208 | C | CYS | A | 265 | 14.109 | 38.533 | 3.287 | 1.00 | 35.47 | C |
| ATOM | 209 | O | CYS | A | 265 | 14.568 | 37.394 | 3.310 | 1.00 | 35.70 | O |
| ATOM | 215 | N | GLU | A | 266 | 12.920 | 38.815 | 2.762 | 1.00 | 35.41 | N |
| ATOM | 216 | CA | GLU | A | 266 | 12.057 | 37.792 | 2.169 | 1.00 | 35.35 | C |
| ATOM | 217 | CB | GLU | A | 266 | 10.660 | 38.372 | 1.931 | 1.00 | 35.27 | C |
| ATOM | 218 | CG | GLU | A | 266 | 9.887 | 38.643 | 3.208 | 1.00 | 35.13 | C |
| ATOM | 219 | CD | GLU | A | 266 | 9.531 | 37.366 | 3.922 | 1.00 | 34.75 | C |
| ATOM | 220 | OE1 | GLU | A | 266 | 10.097 | 37.091 | 5.009 | 1.00 | 33.72 | O |
| ATOM | 221 | OE2 | GLU | A | 266 | 8.703 | 36.626 | 3.362 | 1.00 | 35.80 | O |
| ATOM | 222 | C | GLU | A | 266 | 12.602 | 37.277 | 0.840 | 1.00 | 35.54 | C |
| ATOM | 223 | O | GLU | A | 266 | 12.744 | 38.051 | −0.095 | 1.00 | 35.91 | O |
| ATOM | 230 | N | PRO | A | 267 | 12.881 | 35.981 | 0.732 | 1.00 | 35.85 | N |
| ATOM | 231 | CA | PRO | A | 267 | 13.479 | 35.432 | −0.494 | 1.00 | 36.14 | C |
| ATOM | 232 | CB | PRO | A | 267 | 13.901 | 34.019 | −0.080 | 1.00 | 36.16 | C |
| ATOM | 233 | CG | PRO | A | 267 | 12.969 | 33.650 | 1.016 | 1.00 | 36.22 | C |
| ATOM | 234 | CD | PRO | A | 267 | 12.642 | 34.934 | 1.741 | 1.00 | 36.03 | C |
| ATOM | 235 | C | PRO | A | 267 | 12.508 | 35.379 | −1.671 | 1.00 | 36.43 | C |
| ATOM | 236 | O | PRO | A | 267 | 11.303 | 35.505 | −1.460 | 1.00 | 36.66 | O |
| ATOM | 244 | N | ASP | A | 268 | 13.036 | 35.205 | −2.886 | 1.00 | 36.59 | N |
| ATOM | 245 | CA | ASP | A | 268 | 12.202 | 35.072 | −4.083 | 1.00 | 36.67 | C |
| ATOM | 246 | CB | ASP | A | 268 | 13.042 | 35.177 | −5.372 | 1.00 | 36.75 | C |
| ATOM | 247 | CG | ASP | A | 268 | 12.247 | 35.731 | −6.558 | 1.00 | 37.23 | C |
| ATOM | 248 | OD1 | ASP | A | 268 | 11.582 | 36.780 | −6.399 | 1.00 | 38.06 | O |
| ATOM | 249 | OD2 | ASP | A | 268 | 12.234 | 35.199 | −7.692 | 1.00 | 37.63 | O |
| ATOM | 250 | C | ASP | A | 268 | 11.480 | 33.732 | −4.025 | 1.00 | 36.79 | C |
| ATOM | 251 | O | ASP | A | 268 | 12.092 | 32.679 | −4.235 | 1.00 | 36.90 | O |
| ATOM | 256 | N | GLU | A | 269 | 10.183 | 33.784 | −3.717 | 1.00 | 36.76 | N |
| ATOM | 257 | CA | GLU | A | 269 | 9.342 | 32.585 | −3.627 | 1.00 | 36.63 | C |
| ATOM | 258 | CB | GLU | A | 269 | 7.869 | 32.962 | −3.430 | 1.00 | 36.81 | C |
| ATOM | 259 | CG | GLU | A | 269 | 7.375 | 32.895 | −1.995 | 1.00 | 38.11 | C |
| ATOM | 260 | CD | GLU | A | 269 | 5.861 | 33.078 | −1.891 | 1.00 | 40.19 | C |
| ATOM | 261 | OE1 | GLU | A | 269 | 5.189 | 33.080 | −2.953 | 1.00 | 39.55 | O |
| ATOM | 262 | OE2 | GLU | A | 269 | 5.342 | 33.216 | −0.742 | 1.00 | 42.24 | O |
| ATOM | 263 | C | GLU | A | 269 | 9.464 | 31.664 | −4.851 | 1.00 | 36.33 | C |
| ATOM | 264 | O | GLU | A | 269 | 9.605 | 30.453 | −4.680 | 1.00 | 36.29 | O |
| ATOM | 271 | N | PRO | A | 270 | 9.386 | 32.216 | −6.068 | 1.00 | 35.81 | N |
| ATOM | 272 | CA | PRO | A | 270 | 9.456 | 31.392 | −7.286 | 1.00 | 35.72 | C |
| ATOM | 273 | CB | PRO | A | 270 | 9.381 | 32.428 | −8.418 | 1.00 | 35.67 | C |
| ATOM | 274 | CG | PRO | A | 270 | 8.716 | 33.598 | −7.819 | 1.00 | 35.66 | C |
| ATOM | 275 | CD | PRO | A | 270 | 9.178 | 33.639 | −6.396 | 1.00 | 35.62 | C |
| ATOM | 276 | C | PRO | A | 270 | 10.721 | 30.528 | −7.433 | 1.00 | 35.64 | C |
| ATOM | 277 | O | PRO | A | 270 | 10.646 | 29.469 | −8.058 | 1.00 | 35.87 | O |
| ATOM | 285 | N | GLN | A | 271 | 11.849 | 30.977 | −6.886 | 1.00 | 35.43 | N |
| ATOM | 286 | CA | GLN | A | 271 | 13.108 | 30.238 | −6.982 | 1.00 | 35.29 | C |
| ATOM | 287 | CB | GLN | A | 271 | 14.306 | 31.191 | −6.863 | 1.00 | 35.33 | C |
| ATOM | 288 | CG | GLN | A | 271 | 14.651 | 31.868 | −8.199 | 1.00 | 35.89 | C |
| ATOM | 289 | CD | GLN | A | 271 | 15.692 | 32.974 | −8.085 | 1.00 | 36.39 | C |
| ATOM | 290 | OE1 | GLN | A | 271 | 16.176 | 33.281 | −6.992 | 1.00 | 37.36 | O |
| ATOM | 291 | NE2 | GLN | A | 271 | 16.036 | 33.576 | −9.220 | 1.00 | 36.72 | N |
| ATOM | 292 | C | GLN | A | 271 | 13.186 | 29.123 | −5.942 | 1.00 | 35.19 | C |
| ATOM | 293 | O | GLN | A | 271 | 13.676 | 28.033 | −6.231 | 1.00 | 34.98 | O |
| ATOM | 302 | N | VAL | A | 272 | 12.701 | 29.398 | −4.733 | 1.00 | 35.21 | N |
| ATOM | 303 | CA | VAL | A | 272 | 12.583 | 28.370 | −3.698 | 1.00 | 35.12 | C |
| ATOM | 304 | CB | VAL | A | 272 | 12.025 | 28.964 | −2.376 | 1.00 | 35.21 | C |
| ATOM | 305 | CG1 | VAL | A | 272 | 11.755 | 27.855 | −1.342 | 1.00 | 35.08 | C |
| ATOM | 306 | CG2 | VAL | A | 272 | 12.975 | 30.031 | −1.808 | 1.00 | 35.06 | C |
| ATOM | 307 | C | VAL | A | 272 | 11.652 | 27.245 | −4.184 | 1.00 | 35.04 | C |
| ATOM | 308 | O | VAL | A | 272 | 11.912 | 26.060 | −3.969 | 1.00 | 35.24 | O |
| ATOM | 318 | N | GLN | A | 273 | 10.588 | 27.651 | −4.869 | 1.00 | 34.71 | N |
| ATOM | 319 | CA | GLN | A | 273 | 9.518 | 26.777 | −5.336 | 1.00 | 34.40 | C |
| ATOM | 320 | CB | GLN | A | 273 | 8.449 | 27.662 | −5.977 | 1.00 | 34.54 | C |
| ATOM | 321 | CG | GLN | A | 273 | 7.136 | 27.005 | −6.297 | 1.00 | 34.62 | C |
| ATOM | 322 | CD | GLN | A | 273 | 6.093 | 28.027 | −6.700 | 1.00 | 34.79 | C |
| ATOM | 323 | OE1 | GLN | A | 273 | 5.679 | 28.079 | −7.859 | 1.00 | 34.90 | O |
| ATOM | 324 | NE2 | GLN | A | 273 | 5.681 | 28.860 | −5.747 | 1.00 | 34.57 | N |
| ATOM | 325 | C | GLN | A | 273 | 9.983 | 25.722 | −6.337 | 1.00 | 34.07 | C |
| ATOM | 326 | O | GLN | A | 273 | 9.655 | 24.543 | −6.200 | 1.00 | 33.83 | O |
| ATOM | 335 | N | ALA | A | 274 | 10.739 | 26.157 | −7.343 | 1.00 | 33.91 | N |
| ATOM | 336 | CA | ALA | A | 274 | 11.242 | 25.271 | −8.400 | 1.00 | 33.62 | C |
| ATOM | 337 | CB | ALA | A | 274 | 11.479 | 26.059 | −9.669 | 1.00 | 33.73 | C |
| ATOM | 338 | C | ALA | A | 274 | 12.523 | 24.544 | −7.995 | 1.00 | 33.44 | C |
| ATOM | 339 | O | ALA | A | 274 | 12.847 | 23.503 | −8.557 | 1.00 | 33.36 | O |
| ATOM | 345 | N | LYS | A | 275 | 13.255 | 25.111 | −7.037 | 1.00 | 33.32 | N |
| ATOM | 346 | CA | LYS | A | 275 | 14.398 | 24.439 | −6.417 | 1.00 | 33.26 | C |
| ATOM | 347 | CB | LYS | A | 275 | 15.112 | 25.387 | −5.435 | 1.00 | 33.47 | C |
| ATOM | 348 | CG | LYS | A | 275 | 16.255 | 24.764 | −4.577 | 1.00 | 34.38 | C |
| ATOM | 349 | CD | LYS | A | 275 | 15.813 | 24.388 | −3.120 | 1.00 | 35.18 | C |
| ATOM | 350 | CE | LYS | A | 275 | 15.544 | 25.612 | −2.219 | 1.00 | 35.54 | C |
| ATOM | 351 | NZ | LYS | A | 275 | 15.439 | 25.264 | −0.752 | 1.00 | 35.70 | N |
| ATOM | 352 | C | LYS | A | 275 | 13.928 | 23.176 | −5.694 | 1.00 | 32.95 | C |
| ATOM | 353 | O | LYS | A | 275 | 14.563 | 22.131 | −5.790 | 1.00 | 32.74 | O |
| ATOM | 367 | N | ILE | A | 276 | 12.807 | 23.277 | −4.981 | 1.00 | 32.76 | N |
| ATOM | 368 | CA | ILE | A | 276 | 12.300 | 22.165 | −4.182 | 1.00 | 32.70 | C |
| ATOM | 369 | CB | ILE | A | 276 | 11.345 | 22.666 | −3.077 | 1.00 | 32.87 | C |
| ATOM | 370 | CG1 | ILE | A | 276 | 12.134 | 23.472 | −2.038 | 1.00 | 33.42 | C |
| ATOM | 371 | CD1 | ILE | A | 276 | 11.287 | 24.397 | −1.180 | 1.00 | 34.02 | C |
| ATOM | 372 | CG2 | ILE | A | 276 | 10.636 | 21.496 | −2.400 | 1.00 | 32.89 | C |
| ATOM | 373 | C | ILE | A | 276 | 11.623 | 21.137 | −5.070 | 1.00 | 32.47 | C |
| ATOM | 374 | O | ILE | A | 276 | 11.677 | 19.944 | −4.785 | 1.00 | 32.13 | O |
| ATOM | 386 | N | MET | A | 277 | 10.999 | 21.609 | −6.147 | 1.00 | 32.54 | N |
| ATOM | 387 | CA | MET | A | 277 | 10.365 | 20.735 | −7.136 | 1.00 | 32.48 | C |
| ATOM | 388 | CB | MET | A | 277 | 9.516 | 21.555 | −8.117 | 1.00 | 32.41 | C |
| ATOM | 389 | CG | MET | A | 277 | 8.475 | 20.745 | −8.881 | 1.00 | 32.21 | C |
| ATOM | 390 | SD | MET | A | 277 | 7.224 | 20.013 | −7.805 | 1.00 | 32.33 | S |
| ATOM | 391 | CE | MET | A | 277 | 7.055 | 18.389 | −8.523 | 1.00 | 31.78 | C |
| ATOM | 392 | C | MET | A | 277 | 11.405 | 19.925 | −7.905 | 1.00 | 32.51 | C |
| ATOM | 393 | O | MET | A | 277 | 11.241 | 18.723 | −8.100 | 1.00 | 32.30 | O |
| ATOM | 403 | N | ALA | A | 278 | 12.478 | 20.587 | −8.325 | 1.00 | 32.71 | N |
| ATOM | 404 | CA | ALA | A | 278 | 13.528 | 19.940 | −9.106 | 1.00 | 32.90 | C |
| ATOM | 405 | CB | ALA | A | 278 | 14.528 | 20.969 | −9.595 | 1.00 | 32.95 | C |
| ATOM | 406 | C | ALA | A | 278 | 14.232 | 18.869 | −8.281 | 1.00 | 33.09 | C |
| ATOM | 407 | O | ALA | A | 278 | 14.516 | 17.777 | −8.775 | 1.00 | 33.06 | O |
| ATOM | 413 | N | TYR | A | 279 | 14.504 | 19.198 | −7.022 | 1.00 | 33.37 | N |
| ATOM | 414 | CA | TYR | A | 279 | 15.112 | 18.269 | −6.071 | 1.00 | 33.76 | C |
| ATOM | 415 | CB | TYR | A | 279 | 15.346 | 18.973 | −4.726 | 1.00 | 34.02 | C |
| ATOM | 416 | CG | TYR | A | 279 | 15.514 | 18.044 | −3.540 | 1.00 | 35.08 | C |
| ATOM | 417 | CD1 | TYR | A | 279 | 14.462 | 17.815 | −2.661 | 1.00 | 36.26 | C |
| ATOM | 418 | CE1 | TYR | A | 279 | 14.602 | 16.963 | −1.567 | 1.00 | 37.30 | C |
| ATOM | 419 | CZ | TYR | A | 279 | 15.813 | 16.327 | −1.338 | 1.00 | 37.78 | C |
| ATOM | 420 | OH | TYR | A | 279 | 15.942 | 15.487 | −0.248 | 1.00 | 38.33 | O |
| ATOM | 421 | CE2 | TYR | A | 279 | 16.880 | 16.537 | −2.201 | 1.00 | 37.27 | C |
| ATOM | 422 | CD2 | TYR | A | 279 | 16.723 | 17.394 | −3.299 | 1.00 | 36.43 | C |
| ATOM | 423 | C | TYR | A | 279 | 14.264 | 17.016 | −5.856 | 1.00 | 33.81 | C |
| ATOM | 424 | O | TYR | A | 279 | 14.805 | 15.917 | −5.754 | 1.00 | 33.96 | O |
| ATOM | 434 | N | LEU | A | 280 | 12.943 | 17.184 | −5.783 | 1.00 | 33.82 | N |
| ATOM | 435 | CA | LEU | A | 280 | 12.031 | 16.062 | −5.543 | 1.00 | 33.75 | C |
| ATOM | 436 | CB | LEU | A | 280 | 10.633 | 16.563 | −5.175 | 1.00 | 33.81 | C |
| ATOM | 437 | CG | LEU | A | 280 | 10.490 | 17.123 | −3.760 | 1.00 | 33.80 | C |
| ATOM | 438 | CD1 | LEU | A | 280 | 9.246 | 17.981 | −3.660 | 1.00 | 33.96 | C |
| ATOM | 439 | CD2 | LEU | A | 280 | 10.466 | 16.007 | −2.724 | 1.00 | 33.99 | C |
| ATOM | 440 | C | LEU | A | 280 | 11.940 | 15.152 | −6.752 | 1.00 | 33.84 | C |
| ATOM | 441 | O | LEU | A | 280 | 11.755 | 13.943 | −6.613 | 1.00 | 34.03 | O |
| ATOM | 453 | N | GLN | A | 281 | 12.068 | 15.738 | −7.936 | 1.00 | 33.95 | N |
| ATOM | 454 | CA | GLN | A | 281 | 12.094 | 14.974 | −9.178 | 1.00 | 34.08 | C |
| ATOM | 455 | CB | GLN | A | 281 | 11.973 | 15.913 | −10.382 | 1.00 | 34.08 | C |
| ATOM | 456 | CG | GLN | A | 281 | 10.563 | 16.472 | −10.574 | 1.00 | 34.16 | C |
| ATOM | 457 | CD | GLN | A | 281 | 10.441 | 17.374 | −11.787 | 1.00 | 34.10 | C |
| ATOM | 458 | OE1 | GLN | A | 281 | 10.470 | 18.598 | −11.659 | 1.00 | 34.59 | O |
| ATOM | 459 | NE2 | GLN | A | 281 | 10.301 | 16.775 | −12.962 | 1.00 | 32.93 | N |
| ATOM | 460 | C | GLN | A | 281 | 13.362 | 14.125 | −9.278 | 1.00 | 34.25 | C |
| ATOM | 461 | O | GLN | A | 281 | 13.342 | 13.044 | −9.863 | 1.00 | 34.25 | O |
| ATOM | 470 | N | GLN | A | 282 | 14.449 | 14.614 | −8.684 | 1.00 | 34.59 | N |
| ATOM | 471 | CA | GLN | A | 282 | 15.730 | 13.916 | −8.684 | 1.00 | 34.93 | C |
| ATOM | 472 | CB | GLN | A | 282 | 16.854 | 14.866 | −8.286 | 1.00 | 34.99 | C |
| ATOM | 473 | CG | GLN | A | 282 | 18.220 | 14.371 | −8.732 | 1.00 | 35.44 | C |
| ATOM | 474 | CD | GLN | A | 282 | 18.602 | 14.869 | −10.107 | 1.00 | 35.71 | C |
| ATOM | 475 | OE1 | GLN | A | 282 | 19.324 | 15.863 | −10.232 | 1.00 | 36.78 | O |
| ATOM | 476 | NE2 | GLN | A | 282 | 18.127 | 14.183 | −11.143 | 1.00 | 34.76 | N |
| ATOM | 477 | C | GLN | A | 282 | 15.739 | 12.712 | −7.751 | 1.00 | 35.15 | C |
| ATOM | 478 | O | GLN | A | 282 | 16.192 | 11.626 | −8.126 | 1.00 | 35.40 | O |
| ATOM | 487 | N | GLU | A | 283 | 15.241 | 12.912 | −6.536 | 1.00 | 35.49 | N |
| ATOM | 488 | CA | GLU | A | 283 | 15.036 | 11.824 | −5.577 | 1.00 | 35.86 | C |
| ATOM | 489 | CB | GLU | A | 283 | 14.509 | 12.389 | −4.252 | 1.00 | 35.83 | C |
| ATOM | 490 | CG | GLU | A | 283 | 15.558 | 13.155 | −3.456 | 1.00 | 36.16 | C |
| ATOM | 491 | CD | GLU | A | 283 | 16.041 | 12.413 | −2.216 | 1.00 | 37.22 | C |
| ATOM | 492 | OE1 | GLU | A | 283 | 15.749 | 11.201 | −2.091 | 1.00 | 37.40 | O |
| ATOM | 493 | OE2 | GLU | A | 283 | 16.718 | 13.046 | −1.361 | 1.00 | 37.35 | O |
| ATOM | 494 | C | GLU | A | 283 | 14.075 | 10.740 | −6.098 | 1.00 | 36.16 | C |
| ATOM | 495 | O | GLU | A | 283 | 13.855 | 9.732 | −5.420 | 1.00 | 36.44 | O |
| ATOM | 502 | N | GLN | A | 284 | 13.494 | 10.971 | −7.279 | 1.00 | 36.30 | N |
| ATOM | 503 | CA | GLN | A | 284 | 12.695 | 9.985 | −8.000 | 1.00 | 36.41 | C |
| ATOM | 504 | CB | GLN | A | 284 | 13.512 | 8.719 | −8.301 | 1.00 | 36.29 | C |
| ATOM | 505 | CG | GLN | A | 284 | 14.421 | 8.864 | −9.520 | 1.00 | 36.33 | C |
| ATOM | 506 | CD | GLN | A | 284 | 15.672 | 8.006 | −9.437 | 1.00 | 36.30 | C |
| ATOM | 507 | OE1 | GLN | A | 284 | 15.652 | 6.828 | −9.795 | 1.00 | 36.11 | O |
| ATOM | 508 | NE2 | GLN | A | 284 | 16.760 | 8.595 | −8.963 | 1.00 | 36.48 | N |
| ATOM | 509 | C | GLN | A | 284 | 11.401 | 9.654 | −7.255 | 1.00 | 36.73 | C |
| ATOM | 510 | O | GLN | A | 284 | 10.346 | 9.474 | −7.877 | 1.00 | 37.29 | O |
| ATOM | 519 | N | LYS | A | 292 | 11.505 | 3.201 | −4.037 | 1.00 | 35.08 | N |
| ATOM | 520 | CA | LYS | A | 292 | 10.407 | 3.653 | −4.886 | 1.00 | 35.02 | C |
| ATOM | 521 | CB | LYS | A | 292 | 9.755 | 2.443 | −5.559 | 1.00 | 34.97 | C |
| ATOM | 522 | CG | LYS | A | 292 | 10.801 | 1.462 | −6.143 | 1.00 | 34.79 | C |
| ATOM | 523 | CD | LYS | A | 292 | 10.219 | 0.465 | −7.155 | 1.00 | 34.31 | C |
| ATOM | 524 | CE | LYS | A | 292 | 10.984 | 0.505 | −8.478 | 1.00 | 34.16 | C |
| ATOM | 525 | NZ | LYS | A | 292 | 10.505 | −0.509 | −9.449 | 1.00 | 33.93 | N |
| ATOM | 526 | C | LYS | A | 292 | 9.415 | 4.506 | −4.065 | 1.00 | 35.16 | C |
| ATOM | 527 | O | LYS | A | 292 | 8.318 | 4.065 | −3.716 | 1.00 | 35.29 | O |
| ATOM | 540 | N | LEU | A | 293 | 9.842 | 5.747 | −3.809 | 1.00 | 35.14 | N |
| ATOM | 541 | CA | LEU | A | 293 | 9.260 | 6.688 | −2.831 | 1.00 | 35.15 | C |
| ATOM | 542 | CB | LEU | A | 293 | 9.605 | 8.138 | −3.230 | 1.00 | 35.26 | C |
| ATOM | 543 | CG | LEU | A | 293 | 10.454 | 8.963 | −2.253 | 1.00 | 35.63 | C |
| ATOM | 544 | CD1 | LEU | A | 293 | 10.470 | 10.427 | −2.674 | 1.00 | 35.86 | C |
| ATOM | 545 | CD2 | LEU | A | 293 | 9.973 | 8.847 | −0.818 | 1.00 | 36.21 | C |
| ATOM | 546 | C | LEU | A | 293 | 7.757 | 6.637 | −2.537 | 1.00 | 34.85 | C |
| ATOM | 547 | O | LEU | A | 293 | 6.939 | 6.448 | −3.438 | 1.00 | 34.95 | O |
| ATOM | 559 | N | SER | A | 294 | 7.423 | 6.851 | −1.262 | 1.00 | 34.43 | N |
| ATOM | 560 | CA | SER | A | 294 | 6.044 | 6.884 | −0.780 | 1.00 | 34.12 | C |
| ATOM | 561 | CB | SER | A | 294 | 5.932 | 6.114 | 0.537 | 1.00 | 33.96 | C |
| ATOM | 562 | OG | SER | A | 294 | 5.970 | 6.984 | 1.651 | 1.00 | 33.91 | O |
| ATOM | 563 | C | SER | A | 294 | 5.561 | 8.318 | −0.582 | 1.00 | 34.03 | C |
| ATOM | 564 | O | SER | A | 294 | 6.364 | 9.246 | −0.479 | 1.00 | 34.16 | O |
| ATOM | 570 | N | THR | A | 295 | 4.245 | 8.484 | −0.501 | 1.00 | 33.74 | N |
| ATOM | 571 | CA | THR | A | 295 | 3.616 | 9.803 | −0.432 | 1.00 | 33.71 | C |
| ATOM | 572 | CB | THR | A | 295 | 2.090 | 9.643 | −0.334 | 1.00 | 33.71 | C |
| ATOM | 573 | OG1 | THR | A | 295 | 1.619 | 8.764 | −1.363 | 1.00 | 34.76 | O |
| ATOM | 574 | CG2 | THR | A | 295 | 1.381 | 10.951 | −0.617 | 1.00 | 33.56 | C |
| ATOM | 575 | C | THR | A | 295 | 4.092 | 10.631 | 0.763 | 1.00 | 33.65 | C |
| ATOM | 576 | O | THR | A | 295 | 4.461 | 11.798 | 0.625 | 1.00 | 33.34 | O |
| ATOM | 584 | N | PHE | A | 296 | 4.044 | 10.018 | 1.941 | 1.00 | 33.65 | N |
| ATOM | 585 | CA | PHE | A | 296 | 4.418 | 10.686 | 3.174 | 1.00 | 33.55 | C |
| ATOM | 586 | CB | PHE | A | 296 | 4.020 | 9.840 | 4.387 | 1.00 | 33.35 | C |
| ATOM | 587 | CG | PHE | A | 296 | 4.522 | 10.390 | 5.686 | 1.00 | 33.60 | C |
| ATOM | 588 | CD1 | PHE | A | 296 | 3.858 | 11.434 | 6.305 | 1.00 | 33.45 | C |
| ATOM | 589 | CE1 | PHE | A | 296 | 4.312 | 11.967 | 7.490 | 1.00 | 33.36 | C |
| ATOM | 590 | CZ | PHE | A | 296 | 5.461 | 11.469 | 8.066 | 1.00 | 34.14 | C |
| ATOM | 591 | CE2 | PHE | A | 296 | 6.146 | 10.428 | 7.453 | 1.00 | 34.21 | C |
| ATOM | 592 | CD2 | PHE | A | 296 | 5.678 | 9.898 | 6.266 | 1.00 | 33.98 | C |
| ATOM | 593 | C | PHE | A | 296 | 5.918 | 10.977 | 3.201 | 1.00 | 33.61 | C |
| ATOM | 594 | O | PHE | A | 296 | 6.344 | 11.998 | 3.734 | 1.00 | 33.93 | O |
| ATOM | 604 | N | GLY | A | 297 | 6.714 | 10.073 | 2.646 | 1.00 | 33.59 | N |
| ATOM | 605 | CA | GLY | A | 297 | 8.152 | 10.272 | 2.555 | 1.00 | 33.65 | C |
| ATOM | 606 | C | GLY | A | 297 | 8.517 | 11.443 | 1.661 | 1.00 | 33.54 | C |
| ATOM | 607 | O | GLY | A | 297 | 9.445 | 12.183 | 1.962 | 1.00 | 33.58 | O |
| ATOM | 611 | N | LEU | A | 298 | 7.771 | 11.609 | 0.572 | 1.00 | 33.43 | N |
| ATOM | 612 | CA | LEU | A | 298 | 7.962 | 12.710 | −0.370 | 1.00 | 33.33 | C |
| ATOM | 613 | CB | LEU | A | 298 | 7.033 | 12.519 | −1.580 | 1.00 | 33.47 | C |
| ATOM | 614 | CG | LEU | A | 298 | 7.192 | 13.484 | −2.768 | 1.00 | 34.02 | C |
| ATOM | 615 | CD1 | LEU | A | 298 | 7.998 | 12.858 | −3.909 | 1.00 | 33.98 | C |
| ATOM | 616 | CD2 | LEU | A | 298 | 5.831 | 13.952 | −3.273 | 1.00 | 34.28 | C |
| ATOM | 617 | C | LEU | A | 298 | 7.695 | 14.066 | 0.290 | 1.00 | 33.05 | C |
| ATOM | 618 | O | LEU | A | 298 | 8.392 | 15.041 | 0.025 | 1.00 | 32.81 | O |
| ATOM | 630 | N | MET | A | 299 | 6.686 | 14.111 | 1.155 | 1.00 | 32.85 | N |
| ATOM | 631 | CA | MET | A | 299 | 6.292 | 15.335 | 1.845 | 1.00 | 32.68 | C |
| ATOM | 632 | CB | MET | A | 299 | 4.896 | 15.172 | 2.447 | 1.00 | 32.94 | C |
| ATOM | 633 | CG | MET | A | 299 | 3.789 | 14.987 | 1.434 | 1.00 | 33.15 | C |
| ATOM | 634 | SD | MET | A | 299 | 3.296 | 16.556 | 0.759 | 1.00 | 34.78 | S |
| ATOM | 635 | CE | MET | A | 299 | 4.242 | 16.586 | −0.721 | 1.00 | 34.64 | C |
| ATOM | 636 | C | MET | A | 299 | 7.254 | 15.696 | 2.960 | 1.00 | 32.45 | C |
| ATOM | 637 | O | MET | A | 299 | 7.367 | 16.859 | 3.324 | 1.00 | 32.55 | O |
| ATOM | 647 | N | CYS | A | 300 | 7.924 | 14.696 | 3.523 | 1.00 | 32.20 | N |
| ATOM | 648 | CA | CYS | A | 300 | 8.936 | 14.932 | 4.545 | 1.00 | 32.03 | C |
| ATOM | 649 | CB | CYS | A | 300 | 9.351 | 13.625 | 5.219 | 1.00 | 32.02 | C |
| ATOM | 650 | SG | CYS | A | 300 | 8.111 | 12.993 | 6.354 | 1.00 | 31.80 | S |
| ATOM | 651 | C | CYS | A | 300 | 10.154 | 15.580 | 3.930 | 1.00 | 31.89 | C |
| ATOM | 652 | O | CYS | A | 300 | 10.802 | 16.386 | 4.565 | 1.00 | 31.85 | O |
| ATOM | 658 | N | LYS | A | 301 | 10.454 | 15.197 | 2.694 | 1.00 | 32.14 | N |
| ATOM | 659 | CA | LYS | A | 301 | 11.546 | 15.773 | 1.917 | 1.00 | 32.31 | C |
| ATOM | 660 | CB | LYS | A | 301 | 11.846 | 14.883 | 0.705 | 1.00 | 32.39 | C |
| ATOM | 661 | CG | LYS | A | 301 | 12.430 | 13.516 | 1.069 | 1.00 | 32.92 | C |
| ATOM | 662 | CD | LYS | A | 301 | 12.824 | 12.713 | −0.167 | 1.00 | 34.05 | C |
| ATOM | 663 | CE | LYS | A | 301 | 13.419 | 11.351 | 0.212 | 1.00 | 34.70 | C |
| ATOM | 664 | NZ | LYS | A | 301 | 14.683 | 11.447 | 1.007 | 1.00 | 34.61 | N |
| ATOM | 665 | C | LYS | A | 301 | 11.227 | 17.194 | 1.458 | 1.00 | 32.31 | C |
| ATOM | 666 | O | LYS | A | 301 | 12.121 | 18.019 | 1.345 | 1.00 | 32.38 | O |
| ATOM | 680 | N | MET | A | 302 | 9.950 | 17.467 | 1.199 | 1.00 | 32.46 | N |
| ATOM | 681 | CA | MET | A | 302 | 9.479 | 18.805 | 0.833 | 1.00 | 32.46 | C |
| ATOM | 682 | CB | MET | A | 302 | 8.055 | 18.715 | 0.269 | 1.00 | 32.43 | C |
| ATOM | 683 | CG | MET | A | 302 | 7.426 | 20.058 | −0.092 | 1.00 | 31.98 | C |
| ATOM | 684 | SD | MET | A | 302 | 5.748 | 19.923 | −0.724 | 1.00 | 31.72 | S |
| ATOM | 685 | CE | MET | A | 302 | 5.967 | 18.922 | −2.199 | 1.00 | 31.29 | C |
| ATOM | 686 | C | MET | A | 302 | 9.499 | 19.760 | 2.032 | 1.00 | 32.56 | C |
| ATOM | 687 | O | MET | A | 302 | 9.766 | 20.947 | 1.882 | 1.00 | 32.54 | O |
| ATOM | 697 | N | ALA | A | 303 | 9.179 | 19.241 | 3.213 | 1.00 | 32.72 | N |
| ATOM | 698 | CA | ALA | A | 303 | 9.232 | 20.022 | 4.436 | 1.00 | 32.97 | C |
| ATOM | 699 | CB | ALA | A | 303 | 8.473 | 19.318 | 5.537 | 1.00 | 32.96 | C |
| ATOM | 700 | C | ALA | A | 303 | 10.687 | 20.257 | 4.849 | 1.00 | 33.37 | C |
| ATOM | 701 | O | ALA | A | 303 | 11.038 | 21.322 | 5.340 | 1.00 | 33.52 | O |
| ATOM | 707 | N | ASP | A | 304 | 11.528 | 19.255 | 4.634 | 1.00 | 33.76 | N |
| ATOM | 708 | CA | ASP | A | 304 | 12.944 | 19.320 | 4.978 | 1.00 | 34.10 | C |
| ATOM | 709 | CB | ASP | A | 304 | 13.592 | 17.964 | 4.692 | 1.00 | 34.31 | C |
| ATOM | 710 | CG | ASP | A | 304 | 15.091 | 18.007 | 4.754 | 1.00 | 35.78 | C |
| ATOM | 711 | OD1 | ASP | A | 304 | 15.633 | 18.269 | 5.848 | 1.00 | 36.75 | O |
| ATOM | 712 | OD2 | ASP | A | 304 | 15.816 | 17.777 | 3.758 | 1.00 | 38.38 | O |
| ATOM | 713 | C | ASP | A | 304 | 13.642 | 20.440 | 4.196 | 1.00 | 34.11 | C |
| ATOM | 714 | O | ASP | A | 304 | 14.325 | 21.283 | 4.779 | 1.00 | 34.10 | O |
| ATOM | 719 | N | GLN | A | 305 | 13.437 | 20.451 | 2.882 | 1.00 | 33.97 | N |
| ATOM | 720 | CA | GLN | A | 305 | 14.000 | 21.474 | 2.000 | 1.00 | 33.96 | C |
| ATOM | 721 | CB | GLN | A | 305 | 13.759 | 21.105 | 0.531 | 1.00 | 34.08 | C |
| ATOM | 722 | CG | GLN | A | 305 | 14.721 | 20.082 | −0.011 | 1.00 | 34.87 | C |
| ATOM | 723 | CD | GLN | A | 305 | 16.131 | 20.629 | −0.157 | 1.00 | 37.01 | C |
| ATOM | 724 | OE1 | GLN | A | 305 | 16.354 | 21.634 | −0.841 | 1.00 | 38.44 | O |
| ATOM | 725 | NE2 | GLN | A | 305 | 17.087 | 19.969 | 0.483 | 1.00 | 38.72 | N |
| ATOM | 726 | C | GLN | A | 305 | 13.428 | 22.865 | 2.260 | 1.00 | 33.62 | C |
| ATOM | 727 | O | GLN | A | 305 | 14.093 | 23.864 | 2.024 | 1.00 | 33.70 | O |
| ATOM | 736 | N | THR | A | 306 | 12.186 | 22.928 | 2.718 | 1.00 | 33.47 | N |
| ATOM | 737 | CA | THR | A | 306 | 11.592 | 24.192 | 3.130 | 1.00 | 33.45 | C |
| ATOM | 738 | CB | THR | A | 306 | 10.117 | 23.996 | 3.472 | 1.00 | 33.29 | C |
| ATOM | 739 | OG1 | THR | A | 306 | 9.437 | 23.372 | 2.375 | 1.00 | 32.99 | O |
| ATOM | 740 | CG2 | THR | A | 306 | 9.417 | 25.333 | 3.629 | 1.00 | 33.54 | C |
| ATOM | 741 | C | THR | A | 306 | 12.327 | 24.747 | 4.352 | 1.00 | 33.60 | C |
| ATOM | 742 | O | THR | A | 306 | 12.448 | 25.952 | 4.525 | 1.00 | 33.60 | O |
| ATOM | 750 | N | LEU | A | 307 | 12.815 | 23.849 | 5.193 | 1.00 | 33.77 | N |
| ATOM | 751 | CA | LEU | A | 307 | 13.544 | 24.217 | 6.394 | 1.00 | 34.00 | C |
| ATOM | 752 | CB | LEU | A | 307 | 13.622 | 23.008 | 7.333 | 1.00 | 34.30 | C |
| ATOM | 753 | CG | LEU | A | 307 | 13.884 | 23.226 | 8.820 | 1.00 | 34.74 | C |
| ATOM | 754 | CD1 | LEU | A | 307 | 13.080 | 24.391 | 9.383 | 1.00 | 34.91 | C |
| ATOM | 755 | CD2 | LEU | A | 307 | 13.564 | 21.915 | 9.557 | 1.00 | 35.34 | C |
| ATOM | 756 | C | LEU | A | 307 | 14.944 | 24.748 | 6.068 | 1.00 | 33.69 | C |
| ATOM | 757 | O | LEU | A | 307 | 15.440 | 25.628 | 6.757 | 1.00 | 33.50 | O |
| ATOM | 769 | N | PHE | A | 308 | 15.572 | 24.208 | 5.025 | 1.00 | 33.39 | N |
| ATOM | 770 | CA | PHE | A | 308 | 16.799 | 24.777 | 4.472 | 1.00 | 33.02 | C |
| ATOM | 771 | CB | PHE | A | 308 | 17.254 | 24.014 | 3.224 | 1.00 | 33.09 | C |
| ATOM | 772 | CG | PHE | A | 308 | 18.079 | 22.788 | 3.508 | 1.00 | 33.54 | C |
| ATOM | 773 | CD1 | PHE | A | 308 | 17.526 | 21.693 | 4.150 | 1.00 | 34.65 | C |
| ATOM | 774 | CE1 | PHE | A | 308 | 18.275 | 20.552 | 4.396 | 1.00 | 34.55 | C |
| ATOM | 775 | CZ | PHE | A | 308 | 19.588 | 20.493 | 3.992 | 1.00 | 34.29 | C |
| ATOM | 776 | CE2 | PHE | A | 308 | 20.153 | 21.575 | 3.341 | 1.00 | 34.60 | C |
| ATOM | 777 | CD2 | PHE | A | 308 | 19.398 | 22.714 | 3.097 | 1.00 | 34.14 | C |
| ATOM | 778 | C | PHE | A | 308 | 16.540 | 26.213 | 4.059 | 1.00 | 32.89 | C |
| ATOM | 779 | O | PHE | A | 308 | 17.358 | 27.086 | 4.289 | 1.00 | 33.16 | O |
| ATOM | 789 | N | SER | A | 309 | 15.403 | 26.436 | 3.416 | 1.00 | 32.80 | N |
| ATOM | 790 | CA | SER | A | 309 | 15.008 | 27.755 | 2.943 | 1.00 | 32.70 | C |
| ATOM | 791 | CB | SER | A | 309 | 13.771 | 27.624 | 2.042 | 1.00 | 32.66 | C |
| ATOM | 792 | OG | SER | A | 309 | 13.097 | 28.854 | 1.893 | 1.00 | 32.71 | O |
| ATOM | 793 | C | SER | A | 309 | 14.754 | 28.731 | 4.107 | 1.00 | 32.83 | C |
| ATOM | 794 | O | SER | A | 309 | 15.044 | 29.927 | 3.999 | 1.00 | 33.00 | O |
| ATOM | 800 | N | ILE | A | 310 | 14.230 | 28.219 | 5.219 | 1.00 | 32.78 | N |
| ATOM | 801 | CA | ILE | A | 310 | 13.966 | 29.038 | 6.399 | 1.00 | 32.67 | C |
| ATOM | 802 | CB | ILE | A | 310 | 13.017 | 28.302 | 7.384 | 1.00 | 32.62 | C |
| ATOM | 803 | CG1 | ILE | A | 310 | 11.623 | 28.208 | 6.758 | 1.00 | 32.65 | C |
| ATOM | 804 | CD1 | ILE | A | 310 | 10.511 | 27.832 | 7.692 | 1.00 | 32.71 | C |
| ATOM | 805 | CG2 | ILE | A | 310 | 12.947 | 29.021 | 8.727 | 1.00 | 32.94 | C |
| ATOM | 806 | C | ILE | A | 310 | 15.272 | 29.453 | 7.081 | 1.00 | 32.56 | C |
| ATOM | 807 | O | ILE | A | 310 | 15.416 | 30.612 | 7.480 | 1.00 | 32.17 | O |
| ATOM | 819 | N | VAL | A | 311 | 16.216 | 28.515 | 7.193 | 1.00 | 32.25 | N |
| ATOM | 820 | CA | VAL | A | 311 | 17.505 | 28.783 | 7.832 | 1.00 | 31.95 | C |
| ATOM | 821 | CB | VAL | A | 311 | 18.345 | 27.504 | 8.024 | 1.00 | 31.68 | C |
| ATOM | 822 | CG1 | VAL | A | 311 | 19.729 | 27.841 | 8.564 | 1.00 | 31.83 | C |
| ATOM | 823 | CG2 | VAL | A | 311 | 17.666 | 26.545 | 8.961 | 1.00 | 31.30 | C |
| ATOM | 824 | C | VAL | A | 311 | 18.302 | 29.789 | 7.008 | 1.00 | 32.22 | C |
| ATOM | 825 | O | VAL | A | 311 | 19.033 | 30.597 | 7.566 | 1.00 | 32.24 | O |
| ATOM | 835 | N | GLU | A | 312 | 18.145 | 29.733 | 5.687 | 1.00 | 32.37 | N |
| ATOM | 836 | CA | GLU | A | 312 | 18.802 | 30.650 | 4.764 | 1.00 | 32.69 | C |
| ATOM | 837 | CB | GLU | A | 312 | 18.530 | 30.209 | 3.318 | 1.00 | 32.75 | C |
| ATOM | 838 | CG | GLU | A | 312 | 19.394 | 30.888 | 2.265 | 1.00 | 33.86 | C |
| ATOM | 839 | CD | GLU | A | 312 | 19.164 | 30.343 | 0.855 | 1.00 | 35.82 | C |
| ATOM | 840 | OE1 | GLU | A | 312 | 18.100 | 29.707 | 0.608 | 1.00 | 35.74 | O |
| ATOM | 841 | OE2 | GLU | A | 312 | 20.054 | 30.555 | −0.013 | 1.00 | 36.01 | O |
| ATOM | 842 | C | GLU | A | 312 | 18.306 | 32.079 | 4.975 | 1.00 | 32.72 | C |
| ATOM | 843 | O | GLU | A | 312 | 19.087 | 33.022 | 4.992 | 1.00 | 32.85 | O |
| ATOM | 850 | N | TRP | A | 313 | 16.997 | 32.223 | 5.135 | 1.00 | 33.09 | N |
| ATOM | 851 | CA | TRP | A | 313 | 16.369 | 33.524 | 5.345 | 1.00 | 33.22 | C |
| ATOM | 852 | CB | TRP | A | 313 | 14.841 | 33.394 | 5.311 | 1.00 | 33.27 | C |
| ATOM | 853 | CG | TRP | A | 313 | 14.144 | 34.449 | 6.097 | 1.00 | 33.50 | C |
| ATOM | 854 | CD1 | TRP | A | 313 | 13.846 | 35.702 | 5.679 | 1.00 | 34.11 | C |
| ATOM | 855 | NE1 | TRP | A | 313 | 13.215 | 36.402 | 6.679 | 1.00 | 34.60 | N |
| ATOM | 856 | CE2 | TRP | A | 313 | 13.094 | 35.594 | 7.779 | 1.00 | 33.80 | C |
| ATOM | 857 | CD2 | TRP | A | 313 | 13.674 | 34.355 | 7.450 | 1.00 | 33.38 | C |
| ATOM | 858 | CE3 | TRP | A | 313 | 13.692 | 33.351 | 8.424 | 1.00 | 33.26 | C |
| ATOM | 859 | CZ3 | TRP | A | 313 | 13.129 | 33.610 | 9.671 | 1.00 | 32.69 | C |
| ATOM | 860 | CH2 | TRP | A | 313 | 12.559 | 34.849 | 9.961 | 1.00 | 32.35 | C |
| ATOM | 861 | CZ2 | TRP | A | 313 | 12.532 | 35.853 | 9.033 | 1.00 | 33.05 | C |
| ATOM | 862 | C | TRP | A | 313 | 16.804 | 34.136 | 6.671 | 1.00 | 33.37 | C |
| ATOM | 863 | O | TRP | A | 313 | 17.085 | 35.326 | 6.737 | 1.00 | 34.05 | O |
| ATOM | 874 | N | ALA | A | 314 | 16.852 | 33.318 | 7.718 | 1.00 | 33.35 | N |
| ATOM | 875 | CA | ALA | A | 314 | 17.235 | 33.763 | 9.053 | 1.00 | 33.33 | C |
| ATOM | 876 | CB | ALA | A | 314 | 17.003 | 32.649 | 10.063 | 1.00 | 33.10 | C |
| ATOM | 877 | C | ALA | A | 314 | 18.697 | 34.204 | 9.085 | 1.00 | 33.55 | C |
| ATOM | 878 | O | ALA | A | 314 | 19.053 | 35.191 | 9.746 | 1.00 | 33.26 | O |
| ATOM | 884 | N | ARG | A | 315 | 19.519 | 33.476 | 8.333 | 1.00 | 33.59 | N |
| ATOM | 885 | CA | ARG | A | 315 | 20.973 | 33.629 | 8.336 | 1.00 | 33.77 | C |
| ATOM | 886 | CB | ARG | A | 315 | 21.589 | 32.530 | 7.466 | 1.00 | 33.68 | C |
| ATOM | 887 | CG | ARG | A | 315 | 23.054 | 32.284 | 7.683 | 1.00 | 33.90 | C |
| ATOM | 888 | CD | ARG | A | 315 | 23.650 | 31.368 | 6.641 | 1.00 | 34.20 | C |
| ATOM | 889 | NE | ARG | A | 315 | 25.091 | 31.557 | 6.505 | 1.00 | 34.17 | N |
| ATOM | 890 | CZ | ARG | A | 315 | 25.782 | 31.371 | 5.384 | 1.00 | 34.70 | C |
| ATOM | 891 | NH1 | ARG | A | 315 | 25.183 | 30.983 | 4.261 | 1.00 | 35.10 | N |
| ATOM | 892 | NH2 | ARG | A | 315 | 27.094 | 31.575 | 5.382 | 1.00 | 34.75 | N |
| ATOM | 893 | C | ARG | A | 315 | 21.435 | 34.991 | 7.827 | 1.00 | 33.91 | C |
| ATOM | 894 | O | ARG | A | 315 | 22.496 | 35.485 | 8.220 | 1.00 | 33.87 | O |
| ATOM | 908 | N | SER | A | 316 | 20.648 | 35.582 | 6.935 | 1.00 | 34.21 | N |
| ATOM | 909 | CA | SER | A | 316 | 20.967 | 36.887 | 6.363 | 1.00 | 34.44 | C |
| ATOM | 910 | CB | SER | A | 316 | 21.078 | 36.778 | 4.842 | 1.00 | 34.41 | C |
| ATOM | 911 | OG | SER | A | 316 | 19.929 | 36.159 | 4.296 | 1.00 | 34.54 | O |
| ATOM | 912 | C | SER | A | 316 | 19.906 | 37.916 | 6.757 | 1.00 | 34.73 | C |
| ATOM | 913 | O | SER | A | 316 | 19.713 | 38.915 | 6.066 | 1.00 | 34.97 | O |
| ATOM | 919 | N | SER | A | 317 | 19.227 | 37.663 | 7.875 | 1.00 | 34.90 | N |
| ATOM | 920 | CA | SER | A | 317 | 18.271 | 38.604 | 8.446 | 1.00 | 34.94 | C |
| ATOM | 921 | CB | SER | A | 317 | 17.202 | 37.863 | 9.246 | 1.00 | 35.07 | C |
| ATOM | 922 | OG | SER | A | 317 | 16.437 | 37.015 | 8.404 | 1.00 | 35.41 | O |
| ATOM | 923 | C | SER | A | 317 | 18.994 | 39.603 | 9.338 | 1.00 | 35.00 | C |
| ATOM | 924 | O | SER | A | 317 | 20.104 | 39.341 | 9.790 | 1.00 | 34.71 | O |
| ATOM | 930 | N | ILE | A | 318 | 18.325 | 40.717 | 9.629 | 1.00 | 35.19 | N |
| ATOM | 931 | CA | ILE | A | 318 | 18.972 | 41.927 | 10.149 | 1.00 | 35.26 | C |
| ATOM | 932 | CB | ILE | A | 318 | 17.919 | 43.064 | 10.361 | 1.00 | 35.39 | C |
| ATOM | 933 | CG1 | ILE | A | 318 | 17.236 | 43.428 | 9.037 | 1.00 | 35.49 | C |
| ATOM | 934 | CD1 | ILE | A | 318 | 15.785 | 43.034 | 8.983 | 1.00 | 35.43 | C |
| ATOM | 935 | CG2 | ILE | A | 318 | 18.567 | 44.320 | 10.978 | 1.00 | 35.56 | C |
| ATOM | 936 | C | ILE | A | 318 | 19.759 | 41.712 | 11.441 | 1.00 | 35.15 | C |
| ATOM | 937 | O | ILE | A | 318 | 20.965 | 41.966 | 11.501 | 1.00 | 35.44 | O |
| ATOM | 949 | N | PHE | A | 319 | 19.068 | 41.264 | 12.476 | 1.00 | 34.79 | N |
| ATOM | 950 | CA | PHE | A | 319 | 19.659 | 41.189 | 13.804 | 1.00 | 34.46 | C |
| ATOM | 951 | CB | PHE | A | 319 | 18.600 | 41.582 | 14.836 | 1.00 | 34.70 | C |
| ATOM | 952 | CG | PHE | A | 319 | 17.914 | 42.893 | 14.517 | 1.00 | 35.09 | C |
| ATOM | 953 | CD1 | PHE | A | 319 | 16.690 | 42.915 | 13.857 | 1.00 | 35.60 | C |
| ATOM | 954 | CE1 | PHE | A | 319 | 16.070 | 44.125 | 13.543 | 1.00 | 35.70 | C |
| ATOM | 955 | CZ | PHE | A | 319 | 16.671 | 45.326 | 13.891 | 1.00 | 35.54 | C |
| ATOM | 956 | CE2 | PHE | A | 319 | 17.891 | 45.317 | 14.546 | 1.00 | 35.62 | C |
| ATOM | 957 | CD2 | PHE | A | 319 | 18.511 | 44.104 | 14.850 | 1.00 | 35.50 | C |
| ATOM | 958 | C | PHE | A | 319 | 20.238 | 39.800 | 14.070 | 1.00 | 34.03 | C |
| ATOM | 959 | O | PHE | A | 319 | 21.095 | 39.626 | 14.934 | 1.00 | 33.62 | O |
| ATOM | 969 | N | PHE | A | 320 | 19.770 | 38.824 | 13.298 | 1.00 | 33.69 | N |
| ATOM | 970 | CA | PHE | A | 320 | 20.181 | 37.432 | 13.432 | 1.00 | 33.34 | C |
| ATOM | 971 | CB | PHE | A | 320 | 19.207 | 36.562 | 12.642 | 1.00 | 33.30 | C |
| ATOM | 972 | CG | PHE | A | 320 | 19.223 | 35.120 | 13.024 | 1.00 | 32.96 | C |
| ATOM | 973 | CD1 | PHE | A | 320 | 18.510 | 34.678 | 14.126 | 1.00 | 33.16 | C |
| ATOM | 974 | CE1 | PHE | A | 320 | 18.498 | 33.328 | 14.473 | 1.00 | 32.83 | C |
| ATOM | 975 | CZ | PHE | A | 320 | 19.198 | 32.419 | 13.714 | 1.00 | 32.73 | C |
| ATOM | 976 | CE2 | PHE | A | 320 | 19.911 | 32.852 | 12.603 | 1.00 | 32.89 | C |
| ATOM | 977 | CD2 | PHE | A | 320 | 19.920 | 34.193 | 12.265 | 1.00 | 32.53 | C |
| ATOM | 978 | C | PHE | A | 320 | 21.610 | 37.228 | 12.920 | 1.00 | 33.17 | C |
| ATOM | 979 | O | PHE | A | 320 | 22.388 | 36.483 | 13.508 | 1.00 | 32.85 | O |
| ATOM | 989 | N | ARG | A | 321 | 21.946 | 37.904 | 11.826 | 1.00 | 32.95 | N |
| ATOM | 990 | CA | ARG | A | 321 | 23.296 | 37.879 | 11.286 | 1.00 | 33.11 | C |
| ATOM | 991 | CB | ARG | A | 321 | 23.371 | 38.720 | 10.002 | 1.00 | 33.17 | C |
| ATOM | 992 | CG | ARG | A | 321 | 23.414 | 40.225 | 10.244 | 1.00 | 33.52 | C |
| ATOM | 993 | CD | ARG | A | 321 | 22.599 | 41.060 | 9.272 | 1.00 | 34.15 | C |
| ATOM | 994 | NE | ARG | A | 321 | 23.060 | 40.958 | 7.895 | 1.00 | 35.39 | N |
| ATOM | 995 | CZ | ARG | A | 321 | 22.557 | 41.658 | 6.879 | 1.00 | 36.44 | C |
| ATOM | 996 | NH1 | ARG | A | 321 | 21.556 | 42.519 | 7.069 | 1.00 | 36.12 | N |
| ATOM | 997 | NH2 | ARG | A | 321 | 23.058 | 41.495 | 5.656 | 1.00 | 37.27 | N |
| ATOM | 998 | C | ARG | A | 321 | 24.355 | 38.371 | 12.294 | 1.00 | 33.11 | C |
| ATOM | 999 | O | ARG | A | 321 | 25.536 | 38.053 | 12.146 | 1.00 | 33.13 | O |
| ATOM | 1013 | N | GLU | A | 322 | 23.928 | 39.144 | 13.297 | 1.00 | 32.99 | N |
| ATOM | 1014 | CA | GLU | A | 322 | 24.825 | 39.679 | 14.327 | 1.00 | 33.08 | C |
| ATOM | 1015 | CB | GLU | A | 322 | 24.300 | 41.024 | 14.855 | 1.00 | 33.17 | C |
| ATOM | 1016 | CG | GLU | A | 322 | 24.715 | 42.230 | 14.024 | 1.00 | 33.91 | C |
| ATOM | 1017 | CD | GLU | A | 322 | 23.628 | 43.286 | 13.942 | 1.00 | 34.92 | C |
| ATOM | 1018 | OE1 | GLU | A | 322 | 23.360 | 43.953 | 14.966 | 1.00 | 34.64 | O |
| ATOM | 1019 | OE2 | GLU | A | 322 | 23.037 | 43.441 | 12.850 | 1.00 | 36.18 | O |
| ATOM | 1020 | C | GLU | A | 322 | 25.060 | 38.743 | 15.514 | 1.00 | 32.90 | C |
| ATOM | 1021 | O | GLU | A | 322 | 25.918 | 39.016 | 16.352 | 1.00 | 33.11 | O |
| ATOM | 1028 | N | LEU | A | 323 | 24.303 | 37.658 | 15.602 | 1.00 | 32.84 | N |
| ATOM | 1029 | CA | LEU | A | 323 | 24.478 | 36.692 | 16.687 | 1.00 | 32.83 | C |
| ATOM | 1030 | CB | LEU | A | 323 | 23.190 | 35.891 | 16.927 | 1.00 | 32.77 | C |
| ATOM | 1031 | CG | LEU | A | 323 | 22.023 | 36.661 | 17.557 | 1.00 | 33.34 | C |
| ATOM | 1032 | CD1 | LEU | A | 323 | 20.727 | 35.899 | 17.402 | 1.00 | 33.32 | C |
| ATOM | 1033 | CD2 | LEU | A | 323 | 22.287 | 36.983 | 19.032 | 1.00 | 33.96 | C |
| ATOM | 1034 | C | LEU | A | 323 | 25.622 | 35.731 | 16.397 | 1.00 | 32.73 | C |
| ATOM | 1035 | O | LEU | A | 323 | 25.932 | 35.443 | 15.238 | 1.00 | 32.64 | O |
| ATOM | 1047 | N | LYS | A | 324 | 26.248 | 35.243 | 17.464 | 1.00 | 32.79 | N |
| ATOM | 1048 | CA | LYS | A | 324 | 27.164 | 34.111 | 17.370 | 1.00 | 32.90 | C |
| ATOM | 1049 | CB | LYS | A | 324 | 27.854 | 33.856 | 18.715 | 1.00 | 32.85 | C |
| ATOM | 1050 | CG | LYS | A | 324 | 28.911 | 34.890 | 19.080 | 1.00 | 33.17 | C |
| ATOM | 1051 | CD | LYS | A | 324 | 30.252 | 34.233 | 19.376 | 1.00 | 33.65 | C |
| ATOM | 1052 | CE | LYS | A | 324 | 31.277 | 35.236 | 19.863 | 1.00 | 33.53 | C |
| ATOM | 1053 | NZ | LYS | A | 324 | 31.327 | 35.267 | 21.342 | 1.00 | 34.06 | N |
| ATOM | 1054 | C | LYS | A | 324 | 26.360 | 32.880 | 16.945 | 1.00 | 32.82 | C |
| ATOM | 1055 | O | LYS | A | 324 | 25.171 | 32.795 | 17.232 | 1.00 | 32.69 | O |
| ATOM | 1069 | N | VAL | A | 325 | 27.013 | 31.928 | 16.282 | 1.00 | 32.80 | N |
| ATOM | 1070 | CA | VAL | A | 325 | 26.329 | 30.746 | 15.748 | 1.00 | 32.74 | C |
| ATOM | 1071 | CB | VAL | A | 325 | 27.313 | 29.785 | 15.017 | 1.00 | 32.75 | C |
| ATOM | 1072 | CG1 | VAL | A | 325 | 26.622 | 28.489 | 14.592 | 1.00 | 32.55 | C |
| ATOM | 1073 | CG2 | VAL | A | 325 | 27.916 | 30.460 | 13.801 | 1.00 | 32.95 | C |
| ATOM | 1074 | C | VAL | A | 325 | 25.576 | 29.985 | 16.839 | 1.00 | 32.61 | C |
| ATOM | 1075 | O | VAL | A | 325 | 24.502 | 29.455 | 16.587 | 1.00 | 32.67 | O |
| ATOM | 1085 | N | ASP | A | 326 | 26.127 | 29.948 | 18.049 | 1.00 | 32.56 | N |
| ATOM | 1086 | CA | ASP | A | 326 | 25.507 | 29.219 | 19.159 | 1.00 | 32.58 | C |
| ATOM | 1087 | CB | ASP | A | 326 | 26.466 | 29.141 | 20.347 | 1.00 | 32.73 | C |
| ATOM | 1088 | CG | ASP | A | 326 | 27.677 | 28.286 | 20.052 | 1.00 | 33.51 | C |
| ATOM | 1089 | OD1 | ASP | A | 326 | 28.810 | 28.720 | 20.359 | 1.00 | 33.87 | O |
| ATOM | 1090 | OD2 | ASP | A | 326 | 27.584 | 27.165 | 19.501 | 1.00 | 34.06 | O |
| ATOM | 1091 | C | ASP | A | 326 | 24.180 | 29.831 | 19.602 | 1.00 | 32.23 | C |
| ATOM | 1092 | O | ASP | A | 326 | 23.274 | 29.116 | 20.021 | 1.00 | 32.04 | O |
| ATOM | 1097 | N | ASP | A | 327 | 24.085 | 31.155 | 19.531 | 1.00 | 31.98 | N |
| ATOM | 1098 | CA | ASP | A | 327 | 22.831 | 31.854 | 19.797 | 1.00 | 31.79 | C |
| ATOM | 1099 | CB | ASP | A | 327 | 23.061 | 33.366 | 19.931 | 1.00 | 31.87 | C |
| ATOM | 1100 | CG | ASP | A | 327 | 23.557 | 33.758 | 21.302 | 1.00 | 31.54 | C |
| ATOM | 1101 | OD1 | ASP | A | 327 | 23.827 | 34.958 | 21.518 | 1.00 | 31.29 | O |
| ATOM | 1102 | OD2 | ASP | A | 327 | 23.709 | 32.925 | 22.219 | 1.00 | 31.09 | O |
| ATOM | 1103 | C | ASP | A | 327 | 21.837 | 31.589 | 18.686 | 1.00 | 31.58 | C |
| ATOM | 1104 | O | ASP | A | 327 | 20.660 | 31.360 | 18.945 | 1.00 | 31.34 | O |
| ATOM | 1109 | N | GLN | A | 328 | 22.327 | 31.630 | 17.450 | 1.00 | 31.47 | N |
| ATOM | 1110 | CA | GLN | A | 328 | 21.512 | 31.363 | 16.269 | 1.00 | 31.28 | C |
| ATOM | 1111 | CB | GLN | A | 328 | 22.344 | 31.540 | 14.991 | 1.00 | 31.16 | C |
| ATOM | 1112 | CG | GLN | A | 328 | 22.625 | 32.992 | 14.641 | 1.00 | 30.71 | C |
| ATOM | 1113 | CD | GLN | A | 328 | 23.408 | 33.156 | 13.348 | 1.00 | 31.08 | C |
| ATOM | 1114 | OE1 | GLN | A | 328 | 24.168 | 32.270 | 12.953 | 1.00 | 31.65 | O |
| ATOM | 1115 | NE2 | GLN | A | 328 | 23.236 | 34.296 | 12.694 | 1.00 | 31.31 | N |
| ATOM | 1116 | C | GLN | A | 328 | 20.868 | 29.971 | 16.316 | 1.00 | 31.10 | C |
| ATOM | 1117 | O | GLN | A | 328 | 19.685 | 29.835 | 16.020 | 1.00 | 30.80 | O |
| ATOM | 1126 | N | MET | A | 329 | 21.639 | 28.959 | 16.712 | 1.00 | 31.08 | N |
| ATOM | 1127 | CA | MET | A | 329 | 21.138 | 27.589 | 16.807 | 1.00 | 31.27 | C |
| ATOM | 1128 | CB | MET | A | 329 | 22.282 | 26.616 | 17.087 | 1.00 | 31.15 | C |
| ATOM | 1129 | CG | MET | A | 329 | 23.220 | 26.397 | 15.913 | 1.00 | 31.21 | C |
| ATOM | 1130 | SD | MET | A | 329 | 24.733 | 25.505 | 16.363 | 1.00 | 30.82 | S |
| ATOM | 1131 | CE | MET | A | 329 | 24.099 | 23.850 | 16.733 | 1.00 | 30.51 | C |
| ATOM | 1132 | C | MET | A | 329 | 20.073 | 27.448 | 17.897 | 1.00 | 31.58 | C |
| ATOM | 1133 | O | MET | A | 329 | 19.021 | 26.863 | 17.669 | 1.00 | 31.63 | O |
| ATOM | 1143 | N | LYS | A | 330 | 20.354 | 27.992 | 19.079 | 1.00 | 32.15 | N |
| ATOM | 1144 | CA | LYS | A | 330 | 19.412 | 27.977 | 20.202 | 1.00 | 32.43 | C |
| ATOM | 1145 | CB | LYS | A | 330 | 19.987 | 28.757 | 21.394 | 1.00 | 32.51 | C |
| ATOM | 1146 | CG | LYS | A | 330 | 21.110 | 28.047 | 22.140 | 1.00 | 32.82 | C |
| ATOM | 1147 | CD | LYS | A | 330 | 21.795 | 28.990 | 23.126 | 1.00 | 33.47 | C |
| ATOM | 1148 | CE | LYS | A | 330 | 23.120 | 28.428 | 23.648 | 1.00 | 33.83 | C |
| ATOM | 1149 | NZ | LYS | A | 330 | 24.201 | 29.467 | 23.711 | 1.00 | 32.91 | N |
| ATOM | 1150 | C | LYS | A | 330 | 18.044 | 28.556 | 19.812 | 1.00 | 32.44 | C |
| ATOM | 1151 | O | LYS | A | 330 | 17.005 | 27.964 | 20.101 | 1.00 | 32.44 | O |
| ATOM | 1165 | N | LEU | A | 331 | 18.057 | 29.706 | 19.146 | 1.00 | 32.54 | N |
| ATOM | 1166 | CA | LEU | A | 331 | 16.828 | 30.343 | 18.678 | 1.00 | 32.71 | C |
| ATOM | 1167 | CB | LEU | A | 331 | 17.130 | 31.717 | 18.061 | 1.00 | 32.73 | C |
| ATOM | 1168 | CG | LEU | A | 331 | 17.551 | 32.851 | 19.005 | 1.00 | 32.68 | C |
| ATOM | 1169 | CD1 | LEU | A | 331 | 17.572 | 34.181 | 18.262 | 1.00 | 32.27 | C |
| ATOM | 1170 | CD2 | LEU | A | 331 | 16.644 | 32.937 | 20.232 | 1.00 | 33.07 | C |
| ATOM | 1171 | C | LEU | A | 331 | 16.062 | 29.483 | 17.668 | 1.00 | 32.63 | C |
| ATOM | 1172 | O | LEU | A | 331 | 14.849 | 29.333 | 17.783 | 1.00 | 32.62 | O |
| ATOM | 1184 | N | LEU | A | 332 | 16.770 | 28.914 | 16.696 | 1.00 | 32.70 | N |
| ATOM | 1185 | CA | LEU | A | 332 | 16.130 | 28.137 | 15.624 | 1.00 | 32.70 | C |
| ATOM | 1186 | CB | LEU | A | 332 | 17.047 | 28.024 | 14.402 | 1.00 | 32.64 | C |
| ATOM | 1187 | CG | LEU | A | 332 | 17.132 | 29.237 | 13.468 | 1.00 | 32.81 | C |
| ATOM | 1188 | CD1 | LEU | A | 332 | 18.382 | 29.136 | 12.589 | 1.00 | 32.67 | C |
| ATOM | 1189 | CD2 | LEU | A | 332 | 15.884 | 29.372 | 12.599 | 1.00 | 32.79 | C |
| ATOM | 1190 | C | LEU | A | 332 | 15.678 | 26.738 | 16.080 | 1.00 | 32.77 | C |
| ATOM | 1191 | O | LEU | A | 332 | 14.683 | 26.218 | 15.580 | 1.00 | 32.73 | O |
| ATOM | 1203 | N | GLN | A | 333 | 16.407 | 26.131 | 17.015 | 1.00 | 32.76 | N |
| ATOM | 1204 | CA | GLN | A | 333 | 15.980 | 24.873 | 17.637 | 1.00 | 32.78 | C |
| ATOM | 1205 | CB | GLN | A | 333 | 17.051 | 24.347 | 18.591 | 1.00 | 32.79 | C |
| ATOM | 1206 | CG | GLN | A | 333 | 18.231 | 23.688 | 17.909 | 1.00 | 33.64 | C |
| ATOM | 1207 | CD | GLN | A | 333 | 19.427 | 23.533 | 18.834 | 1.00 | 35.25 | C |
| ATOM | 1208 | OE1 | GLN | A | 333 | 19.868 | 24.504 | 19.452 | 1.00 | 37.23 | O |
| ATOM | 1209 | NE2 | GLN | A | 333 | 19.953 | 22.319 | 18.932 | 1.00 | 36.48 | N |
| ATOM | 1210 | C | GLN | A | 333 | 14.673 | 25.058 | 18.407 | 1.00 | 32.63 | C |
| ATOM | 1211 | O | GLN | A | 333 | 13.914 | 24.114 | 18.595 | 1.00 | 32.48 | O |
| ATOM | 1220 | N | ASN | A | 334 | 14.434 | 26.283 | 18.861 | 1.00 | 32.57 | N |
| ATOM | 1221 | CA | ASN | A | 334 | 13.229 | 26.629 | 19.588 | 1.00 | 32.32 | C |
| ATOM | 1222 | CB | ASN | A | 334 | 13.504 | 27.847 | 20.474 | 1.00 | 32.52 | C |
| ATOM | 1223 | CG | ASN | A | 334 | 12.289 | 28.281 | 21.262 | 1.00 | 32.58 | C |
| ATOM | 1224 | OD1 | ASN | A | 334 | 11.741 | 27.504 | 22.037 | 1.00 | 32.87 | O |
| ATOM | 1225 | ND2 | ASN | A | 334 | 11.853 | 29.521 | 21.059 | 1.00 | 31.96 | N |
| ATOM | 1226 | C | ASN | A | 334 | 12.030 | 26.918 | 18.691 | 1.00 | 32.14 | C |
| ATOM | 1227 | O | ASN | A | 334 | 10.909 | 26.704 | 19.116 | 1.00 | 32.55 | O |
| ATOM | 1234 | N | CYS | A | 335 | 12.258 | 27.391 | 17.463 | 1.00 | 31.90 | N |
| ATOM | 1235 | CA | CYS | A | 335 | 11.178 | 27.950 | 16.630 | 1.00 | 31.50 | C |
| ATOM | 1236 | CB | CYS | A | 335 | 11.308 | 29.479 | 16.571 | 1.00 | 31.43 | C |
| ATOM | 1237 | SG | CYS | A | 335 | 12.477 | 30.095 | 15.341 | 1.00 | 30.63 | S |
| ATOM | 1238 | C | CYS | A | 335 | 11.075 | 27.415 | 15.198 | 1.00 | 31.40 | C |
| ATOM | 1239 | O | CYS | A | 335 | 10.334 | 27.976 | 14.391 | 1.00 | 30.88 | O |
| ATOM | 1245 | N | TRP | A | 336 | 11.796 | 26.342 | 14.884 | 1.00 | 31.31 | N |
| ATOM | 1246 | CA | TRP | A | 336 | 11.871 | 25.843 | 13.506 | 1.00 | 31.08 | C |
| ATOM | 1247 | CB | TRP | A | 336 | 12.893 | 24.697 | 13.369 | 1.00 | 31.04 | C |
| ATOM | 1248 | CG | TRP | A | 336 | 12.571 | 23.473 | 14.186 | 1.00 | 30.52 | C |
| ATOM | 1249 | CD1 | TRP | A | 336 | 12.866 | 23.268 | 15.496 | 1.00 | 30.03 | C |
| ATOM | 1250 | NE1 | TRP | A | 336 | 12.408 | 22.040 | 15.898 | 1.00 | 31.07 | N |
| ATOM | 1251 | CE2 | TRP | A | 336 | 11.801 | 21.421 | 14.837 | 1.00 | 31.48 | C |
| ATOM | 1252 | CD2 | TRP | A | 336 | 11.886 | 22.296 | 13.741 | 1.00 | 30.50 | C |
| ATOM | 1253 | CE3 | TRP | A | 336 | 11.333 | 21.892 | 12.523 | 1.00 | 31.09 | C |
| ATOM | 1254 | CZ3 | TRP | A | 336 | 10.724 | 20.651 | 12.441 | 1.00 | 31.91 | C |
| ATOM | 1255 | CH2 | TRP | A | 336 | 10.653 | 19.805 | 13.550 | 1.00 | 32.27 | C |
| ATOM | 1256 | CZ2 | TRP | A | 336 | 11.186 | 20.168 | 14.754 | 1.00 | 32.14 | C |
| ATOM | 1257 | C | TRP | A | 336 | 10.507 | 25.394 | 13.006 | 1.00 | 31.15 | C |
| ATOM | 1258 | O | TRP | A | 336 | 10.126 | 25.681 | 11.870 | 1.00 | 30.79 | O |
| ATOM | 1269 | N | SER | A | 337 | 9.771 | 24.700 | 13.866 | 1.00 | 31.38 | N |
| ATOM | 1270 | CA | SER | A | 337 | 8.479 | 24.144 | 13.480 | 1.00 | 31.62 | C |
| ATOM | 1271 | CB | SER | A | 337 | 8.061 | 22.997 | 14.400 | 1.00 | 31.24 | C |
| ATOM | 1272 | OG | SER | A | 337 | 8.016 | 23.414 | 15.744 | 1.00 | 31.53 | O |
| ATOM | 1273 | C | SER | A | 337 | 7.416 | 25.232 | 13.442 | 1.00 | 31.59 | C |
| ATOM | 1274 | O | SER | A | 337 | 6.498 | 25.149 | 12.641 | 1.00 | 31.76 | O |
| ATOM | 1280 | N | GLU | A | 338 | 7.551 | 26.254 | 14.286 | 1.00 | 31.73 | N |
| ATOM | 1281 | CA | GLU | A | 338 | 6.637 | 27.406 | 14.252 | 1.00 | 31.70 | C |
| ATOM | 1282 | CB | GLU | A | 338 | 6.888 | 28.340 | 15.416 | 1.00 | 31.60 | C |
| ATOM | 1283 | CG | GLU | A | 338 | 6.530 | 27.763 | 16.756 | 1.00 | 32.19 | C |
| ATOM | 1284 | CD | GLU | A | 338 | 6.753 | 28.765 | 17.853 | 1.00 | 32.93 | C |
| ATOM | 1285 | OE1 | GLU | A | 338 | 5.756 | 29.380 | 18.308 | 1.00 | 34.01 | O |
| ATOM | 1286 | OE2 | GLU | A | 338 | 7.930 | 28.949 | 18.236 | 1.00 | 33.13 | O |
| ATOM | 1287 | C | GLU | A | 338 | 6.795 | 28.204 | 12.972 | 1.00 | 31.65 | C |
| ATOM | 1288 | O | GLU | A | 338 | 5.817 | 28.678 | 12.407 | 1.00 | 31.78 | O |
| ATOM | 1295 | N | LEU | A | 339 | 8.035 | 28.353 | 12.528 | 1.00 | 31.51 | N |
| ATOM | 1296 | CA | LEU | A | 339 | 8.328 | 29.027 | 11.282 | 1.00 | 31.41 | C |
| ATOM | 1297 | CB | LEU | A | 339 | 9.835 | 29.255 | 11.133 | 1.00 | 31.41 | C |
| ATOM | 1298 | CG | LEU | A | 339 | 10.431 | 30.380 | 11.983 | 1.00 | 30.89 | C |
| ATOM | 1299 | CD1 | LEU | A | 339 | 11.892 | 30.523 | 11.698 | 1.00 | 30.75 | C |
| ATOM | 1300 | CD2 | LEU | A | 339 | 9.710 | 31.695 | 11.737 | 1.00 | 30.69 | C |
| ATOM | 1301 | C | LEU | A | 339 | 7.788 | 28.259 | 10.088 | 1.00 | 31.54 | C |
| ATOM | 1302 | O | LEU | A | 339 | 7.380 | 28.860 | 9.119 | 1.00 | 31.64 | O |
| ATOM | 1314 | N | LEU | A | 340 | 7.778 | 26.932 | 10.155 | 1.00 | 31.94 | N |
| ATOM | 1315 | CA | LEU | A | 340 | 7.245 | 26.126 | 9.056 | 1.00 | 31.87 | C |
| ATOM | 1316 | CB | LEU | A | 340 | 7.622 | 24.654 | 9.212 | 1.00 | 31.97 | C |
| ATOM | 1317 | CG | LEU | A | 340 | 9.031 | 24.250 | 8.781 | 1.00 | 32.44 | C |
| ATOM | 1318 | CD1 | LEU | A | 340 | 9.288 | 22.835 | 9.238 | 1.00 | 33.44 | C |
| ATOM | 1319 | CD2 | LEU | A | 340 | 9.221 | 24.364 | 7.274 | 1.00 | 33.15 | C |
| ATOM | 1320 | C | LEU | A | 340 | 5.738 | 26.253 | 9.002 | 1.00 | 31.86 | C |
| ATOM | 1321 | O | LEU | A | 340 | 5.167 | 26.358 | 7.939 | 1.00 | 31.57 | O |
| ATOM | 1333 | N | ILE | A | 341 | 5.106 | 26.241 | 10.168 | 1.00 | 32.25 | N |
| ATOM | 1334 | CA | ILE | A | 341 | 3.662 | 26.390 | 10.275 | 1.00 | 32.48 | C |
| ATOM | 1335 | CB | ILE | A | 341 | 3.223 | 26.137 | 11.729 | 1.00 | 32.47 | C |
| ATOM | 1336 | CG1 | ILE | A | 341 | 3.354 | 24.646 | 12.063 | 1.00 | 32.92 | C |
| ATOM | 1337 | CD1 | ILE | A | 341 | 2.600 | 23.734 | 11.147 | 1.00 | 33.53 | C |
| ATOM | 1338 | CG2 | ILE | A | 341 | 1.796 | 26.655 | 11.997 | 1.00 | 32.64 | C |
| ATOM | 1339 | C | ILE | A | 341 | 3.222 | 27.773 | 9.814 | 1.00 | 32.72 | C |
| ATOM | 1340 | O | ILE | A | 341 | 2.292 | 27.889 | 9.049 | 1.00 | 33.36 | O |
| ATOM | 1352 | N | LEU | A | 342 | 3.896 | 28.812 | 10.285 | 1.00 | 32.86 | N |
| ATOM | 1353 | CA | LEU | A | 342 | 3.582 | 30.187 | 9.916 | 1.00 | 32.92 | C |
| ATOM | 1354 | CB | LEU | A | 342 | 4.502 | 31.147 | 10.690 | 1.00 | 32.79 | C |
| ATOM | 1355 | CG | LEU | A | 342 | 4.244 | 32.657 | 10.743 | 1.00 | 32.45 | C |
| ATOM | 1356 | CD1 | LEU | A | 342 | 2.786 | 33.003 | 10.830 | 1.00 | 32.56 | C |
| ATOM | 1357 | CD2 | LEU | A | 342 | 4.966 | 33.247 | 11.931 | 1.00 | 32.87 | C |
| ATOM | 1358 | C | LEU | A | 342 | 3.747 | 30.356 | 8.403 | 1.00 | 33.37 | C |
| ATOM | 1359 | O | LEU | A | 342 | 2.897 | 30.935 | 7.727 | 1.00 | 33.46 | O |
| ATOM | 1371 | N | ASP | A | 343 | 4.843 | 29.817 | 7.886 | 1.00 | 33.57 | N |
| ATOM | 1372 | CA | ASP | A | 343 | 5.119 | 29.789 | 6.461 | 1.00 | 33.91 | C |
| ATOM | 1373 | CB | ASP | A | 343 | 6.443 | 29.058 | 6.251 | 1.00 | 34.09 | C |
| ATOM | 1374 | CG | ASP | A | 343 | 6.894 | 29.039 | 4.812 | 1.00 | 36.03 | C |
| ATOM | 1375 | OD1 | ASP | A | 343 | 6.949 | 27.928 | 4.237 | 1.00 | 37.42 | O |
| ATOM | 1376 | OD2 | ASP | A | 343 | 7.254 | 30.067 | 4.188 | 1.00 | 37.97 | O |
| ATOM | 1377 | C | ASP | A | 343 | 3.973 | 29.109 | 5.695 | 1.00 | 33.87 | C |
| ATOM | 1378 | O | ASP | A | 343 | 3.504 | 29.622 | 4.690 | 1.00 | 33.94 | O |
| ATOM | 1383 | N | HIS | A | 344 | 3.506 | 27.973 | 6.202 | 1.00 | 33.95 | N |
| ATOM | 1384 | CA | HIS | A | 344 | 2.438 | 27.202 | 5.568 | 1.00 | 33.73 | C |
| ATOM | 1385 | CB | HIS | A | 344 | 2.314 | 25.813 | 6.223 | 1.00 | 33.59 | C |
| ATOM | 1386 | CG | HIS | A | 344 | 1.050 | 25.081 | 5.871 | 1.00 | 33.64 | C |
| ATOM | 1387 | ND1 | HIS | A | 344 | 0.009 | 24.682 | 6.639 | 1.00 | 33.71 | N |
| ATOM | 1388 | CE1 | HIS | A | 344 | −0.891 | 24.056 | 5.811 | 1.00 | 33.42 | C |
| ATOM | 1389 | NE2 | HIS | A | 344 | −0.418 | 24.065 | 4.579 | 1.00 | 33.62 | N |
| ATOM | 1390 | CD2 | HIS | A | 344 | 0.753 | 24.677 | 4.587 | 1.00 | 33.15 | C |
| ATOM | 1391 | C | HIS | A | 344 | 1.112 | 27.955 | 5.645 | 1.00 | 33.74 | C |
| ATOM | 1392 | O | HIS | A | 344 | 0.393 | 28.048 | 4.659 | 1.00 | 33.67 | O |
| ATOM | 1401 | N | ILE | A | 345 | 0.820 | 28.509 | 6.817 | 1.00 | 33.93 | N |
| ATOM | 1402 | CA | ILE | A | 345 | −0.419 | 29.240 | 7.087 | 1.00 | 34.27 | C |
| ATOM | 1403 | CB | ILE | A | 345 | −0.486 | 29.614 | 8.610 | 1.00 | 34.58 | C |
| ATOM | 1404 | CG1 | ILE | A | 345 | −1.316 | 28.599 | 9.396 | 1.00 | 35.19 | C |
| ATOM | 1405 | CD1 | ILE | A | 345 | −0.853 | 27.166 | 9.282 | 1.00 | 36.16 | C |
| ATOM | 1406 | CG2 | ILE | A | 345 | −1.097 | 30.995 | 8.839 | 1.00 | 35.28 | C |
| ATOM | 1407 | C | ILE | A | 345 | −0.552 | 30.493 | 6.211 | 1.00 | 34.16 | C |
| ATOM | 1408 | O | ILE | A | 345 | −1.632 | 30.781 | 5.708 | 1.00 | 34.11 | O |
| ATOM | 1420 | N | TYR | A | 346 | 0.545 | 31.234 | 6.054 | 1.00 | 34.07 | N |
| ATOM | 1421 | CA | TYR | A | 346 | 0.577 | 32.458 | 5.246 | 1.00 | 33.99 | C |
| ATOM | 1422 | CB | TYR | A | 346 | 1.844 | 33.279 | 5.560 | 1.00 | 34.01 | C |
| ATOM | 1423 | CG | TYR | A | 346 | 2.011 | 34.517 | 4.706 | 1.00 | 33.78 | C |
| ATOM | 1424 | CD1 | TYR | A | 346 | 2.943 | 34.553 | 3.681 | 1.00 | 33.86 | C |
| ATOM | 1425 | CE1 | TYR | A | 346 | 3.089 | 35.671 | 2.887 | 1.00 | 33.62 | C |
| ATOM | 1426 | CZ | TYR | A | 346 | 2.301 | 36.774 | 3.109 | 1.00 | 33.41 | C |
| ATOM | 1427 | OH | TYR | A | 346 | 2.455 | 37.881 | 2.304 | 1.00 | 35.01 | O |
| ATOM | 1428 | CE2 | TYR | A | 346 | 1.365 | 36.769 | 4.116 | 1.00 | 33.50 | C |
| ATOM | 1429 | CD2 | TYR | A | 346 | 1.222 | 35.643 | 4.909 | 1.00 | 33.75 | C |
| ATOM | 1430 | C | TYR | A | 346 | 0.489 | 32.141 | 3.748 | 1.00 | 34.04 | C |
| ATOM | 1431 | O | TYR | A | 346 | 0.001 | 32.954 | 2.965 | 1.00 | 34.12 | O |
| ATOM | 1441 | N | ARG | A | 347 | 0.948 | 30.954 | 3.359 | 1.00 | 34.16 | N |
| ATOM | 1442 | CA | ARG | A | 347 | 0.784 | 30.463 | 1.988 | 1.00 | 34.03 | C |
| ATOM | 1443 | CB | ARG | A | 347 | 1.634 | 29.207 | 1.745 | 1.00 | 34.21 | C |
| ATOM | 1444 | CG | ARG | A | 347 | 2.540 | 29.294 | 0.505 | 1.00 | 35.09 | C |
| ATOM | 1445 | CD | ARG | A | 347 | 3.120 | 27.956 | 0.035 | 1.00 | 35.44 | C |
| ATOM | 1446 | NE | ARG | A | 347 | 3.551 | 27.129 | 1.157 | 1.00 | 36.44 | N |
| ATOM | 1447 | CZ | ARG | A | 347 | 4.673 | 27.300 | 1.852 | 1.00 | 35.95 | C |
| ATOM | 1448 | NH1 | ARG | A | 347 | 5.530 | 28.270 | 1.546 | 1.00 | 36.38 | N |
| ATOM | 1449 | NH2 | ARG | A | 347 | 4.937 | 26.483 | 2.862 | 1.00 | 35.51 | N |
| ATOM | 1450 | C | ARG | A | 347 | −0.685 | 30.161 | 1.681 | 1.00 | 33.69 | C |
| ATOM | 1451 | O | ARG | A | 347 | −1.130 | 30.346 | 0.552 | 1.00 | 33.51 | O |
| ATOM | 1465 | N | GLN | A | 348 | −1.432 | 29.703 | 2.685 | 1.00 | 33.40 | N |
| ATOM | 1466 | CA | GLN | A | 348 | −2.856 | 29.401 | 2.511 | 1.00 | 33.15 | C |
| ATOM | 1467 | CB | GLN | A | 348 | −3.388 | 28.478 | 3.618 | 1.00 | 32.81 | C |
| ATOM | 1468 | CG | GLN | A | 348 | −2.580 | 27.217 | 3.895 | 1.00 | 31.97 | C |
| ATOM | 1469 | CD | GLN | A | 348 | −2.118 | 26.487 | 2.642 | 1.00 | 30.80 | C |
| ATOM | 1470 | OE1 | GLN | A | 348 | −2.928 | 25.916 | 1.930 | 1.00 | 30.63 | O |
| ATOM | 1471 | NE2 | GLN | A | 348 | −0.816 | 26.485 | 2.393 | 1.00 | 29.21 | N |
| ATOM | 1472 | C | GLN | A | 348 | −3.716 | 30.661 | 2.468 | 1.00 | 33.27 | C |
| ATOM | 1473 | O | GLN | A | 348 | −4.767 | 30.650 | 1.847 | 1.00 | 33.65 | O |
| ATOM | 1482 | N | VAL | A | 349 | −3.283 | 31.735 | 3.124 | 1.00 | 33.41 | N |
| ATOM | 1483 | CA | VAL | A | 349 | −4.031 | 32.999 | 3.111 | 1.00 | 33.61 | C |
| ATOM | 1484 | CB | VAL | A | 349 | −3.498 | 34.010 | 4.181 | 1.00 | 33.65 | C |
| ATOM | 1485 | CG1 | VAL | A | 349 | −4.232 | 35.341 | 4.090 | 1.00 | 33.49 | C |
| ATOM | 1486 | CG2 | VAL | A | 349 | −3.631 | 33.436 | 5.598 | 1.00 | 33.59 | C |
| ATOM | 1487 | C | VAL | A | 349 | −3.971 | 33.647 | 1.722 | 1.00 | 33.75 | C |
| ATOM | 1488 | O | VAL | A | 349 | −4.999 | 34.016 | 1.153 | 1.00 | 33.66 | O |
| ATOM | 1498 | N | VAL | A | 350 | −2.759 | 33.756 | 1.183 | 1.00 | 34.03 | N |
| ATOM | 1499 | CA | VAL | A | 350 | −2.507 | 34.416 | −0.101 | 1.00 | 34.21 | C |
| ATOM | 1500 | CB | VAL | A | 350 | −0.993 | 34.705 | −0.287 | 1.00 | 34.19 | C |
| ATOM | 1501 | CG1 | VAL | A | 350 | −0.718 | 35.284 | −1.674 | 1.00 | 34.47 | C |
| ATOM | 1502 | CG2 | VAL | A | 350 | −0.471 | 35.635 | 0.812 | 1.00 | 33.80 | C |
| ATOM | 1503 | C | VAL | A | 350 | −2.984 | 33.577 | −1.291 | 1.00 | 34.42 | C |
| ATOM | 1504 | O | VAL | A | 350 | −3.853 | 34.003 | −2.049 | 1.00 | 34.59 | O |
| ATOM | 1514 | N | HIS | A | 351 | −2.407 | 32.386 | −1.441 | 1.00 | 34.69 | N |
| ATOM | 1515 | CA | HIS | A | 351 | −2.640 | 31.524 | −2.607 | 1.00 | 34.82 | C |
| ATOM | 1516 | CB | HIS | A | 351 | −1.348 | 30.780 | −2.963 | 1.00 | 34.90 | C |
| ATOM | 1517 | CG | HIS | A | 351 | −0.218 | 31.689 | −3.333 | 1.00 | 35.26 | C |
| ATOM | 1518 | ND1 | HIS | A | 351 | 0.934 | 31.789 | −2.582 | 1.00 | 35.29 | N |
| ATOM | 1519 | CE1 | HIS | A | 351 | 1.745 | 32.670 | −3.141 | 1.00 | 35.54 | C |
| ATOM | 1520 | NE2 | HIS | A | 351 | 1.159 | 33.149 | −4.224 | 1.00 | 35.61 | N |
| ATOM | 1521 | CD2 | HIS | A | 351 | −0.072 | 32.555 | −4.365 | 1.00 | 35.51 | C |
| ATOM | 1522 | C | HIS | A | 351 | −3.779 | 30.512 | −2.452 | 1.00 | 34.84 | C |
| ATOM | 1523 | O | HIS | A | 351 | −4.332 | 30.047 | −3.455 | 1.00 | 35.12 | O |
| ATOM | 1532 | N | GLY | A | 352 | −4.116 | 30.157 | −1.215 | 1.00 | 34.85 | N |
| ATOM | 1533 | CA | GLY | A | 352 | −5.173 | 29.193 | −0.955 | 1.00 | 34.93 | C |
| ATOM | 1534 | C | GLY | A | 352 | −6.541 | 29.618 | −1.473 | 1.00 | 35.05 | C |
| ATOM | 1535 | O | GLY | A | 352 | −6.881 | 30.805 | −1.483 | 1.00 | 35.03 | O |
| ATOM | 1539 | N | LYS | A | 353 | −7.321 | 28.632 | −1.912 | 1.00 | 35.18 | N |
| ATOM | 1540 | CA | LYS | A | 353 | −8.673 | 28.855 | −2.428 | 1.00 | 35.27 | C |
| ATOM | 1541 | CB | LYS | A | 353 | −8.710 | 28.613 | −3.945 | 1.00 | 35.11 | C |
| ATOM | 1542 | CG | LYS | A | 353 | −7.609 | 29.341 | −4.722 | 1.00 | 35.01 | C |
| ATOM | 1543 | CD | LYS | A | 353 | −8.051 | 29.730 | −6.135 | 1.00 | 34.85 | C |
| ATOM | 1544 | CE | LYS | A | 353 | −8.764 | 31.081 | −6.159 | 1.00 | 34.87 | C |
| ATOM | 1545 | NZ | LYS | A | 353 | −9.791 | 31.172 | −7.237 | 1.00 | 34.58 | N |
| ATOM | 1546 | C | LYS | A | 353 | −9.651 | 27.935 | −1.682 | 1.00 | 35.44 | C |
| ATOM | 1547 | O | LYS | A | 353 | −9.369 | 27.531 | −0.546 | 1.00 | 35.70 | O |
| ATOM | 1561 | N | GLU | A | 354 | −10.792 | 27.623 | −2.303 | 1.00 | 35.46 | N |
| ATOM | 1562 | CA | GLU | A | 354 | −11.815 | 26.765 | −1.692 | 1.00 | 35.38 | C |
| ATOM | 1563 | CB | GLU | A | 354 | −13.168 | 26.945 | −2.404 | 1.00 | 35.36 | C |
| ATOM | 1564 | CG | GLU | A | 354 | −14.215 | 25.868 | −2.108 | 1.00 | 35.30 | C |
| ATOM | 1565 | CD | GLU | A | 354 | −15.121 | 26.221 | −0.944 | 1.00 | 35.60 | C |
| ATOM | 1566 | OE1 | GLU | A | 354 | −16.315 | 26.498 | −1.194 | 1.00 | 35.89 | O |
| ATOM | 1567 | OE2 | GLU | A | 354 | −14.647 | 26.215 | 0.219 | 1.00 | 35.35 | O |
| ATOM | 1568 | C | GLU | A | 354 | −11.415 | 25.287 | −1.710 | 1.00 | 35.31 | C |
| ATOM | 1569 | O | GLU | A | 354 | −11.394 | 24.654 | −2.773 | 1.00 | 35.26 | O |
| ATOM | 1576 | N | GLY | A | 355 | −11.126 | 24.746 | −0.526 | 1.00 | 35.19 | N |
| ATOM | 1577 | CA | GLY | A | 355 | −10.839 | 23.330 | −0.365 | 1.00 | 35.11 | C |
| ATOM | 1578 | C | GLY | A | 355 | −9.633 | 22.889 | −1.173 | 1.00 | 35.02 | C |
| ATOM | 1579 | O | GLY | A | 355 | −9.748 | 22.034 | −2.053 | 1.00 | 34.80 | O |
| ATOM | 1583 | N | SER | A | 356 | −8.478 | 23.485 | −0.876 | 1.00 | 34.96 | N |
| ATOM | 1584 | CA | SER | A | 356 | −7.244 | 23.186 | −1.596 | 1.00 | 34.87 | C |
| ATOM | 1585 | CB | SER | A | 356 | −7.285 | 23.799 | −3.000 | 1.00 | 34.99 | C |
| ATOM | 1586 | OG | SER | A | 356 | −7.500 | 25.200 | −2.934 | 1.00 | 35.49 | O |
| ATOM | 1587 | C | SER | A | 356 | −6.026 | 23.722 | −0.861 | 1.00 | 34.59 | C |
| ATOM | 1588 | O | SER | A | 356 | −5.821 | 24.934 | −0.812 | 1.00 | 34.92 | O |
| ATOM | 1594 | N | ILE | A | 357 | −5.221 | 22.820 | −0.303 | 1.00 | 34.11 | N |
| ATOM | 1595 | CA | ILE | A | 357 | −3.946 | 23.194 | 0.304 | 1.00 | 33.79 | C |
| ATOM | 1596 | CB | ILE | A | 357 | −3.363 | 22.014 | 1.140 | 1.00 | 33.85 | C |
| ATOM | 1597 | CG1 | ILE | A | 357 | −4.358 | 21.514 | 2.206 | 1.00 | 34.33 | C |
| ATOM | 1598 | CD1 | ILE | A | 357 | −4.925 | 22.585 | 3.130 | 1.00 | 34.72 | C |
| ATOM | 1599 | CG2 | ILE | A | 357 | −2.046 | 22.411 | 1.777 | 1.00 | 33.26 | C |
| ATOM | 1600 | C | ILE | A | 357 | −2.942 | 23.600 | −0.779 | 1.00 | 33.45 | C |
| ATOM | 1601 | O | ILE | A | 357 | −2.601 | 22.792 | −1.639 | 1.00 | 33.37 | O |
| ATOM | 1613 | N | PHE | A | 358 | −2.486 | 24.852 | −0.746 | 1.00 | 33.22 | N |
| ATOM | 1614 | CA | PHE | A | 358 | −1.397 | 25.309 | −1.615 | 1.00 | 33.03 | C |
| ATOM | 1615 | CB | PHE | A | 358 | −1.456 | 26.827 | −1.805 | 1.00 | 32.96 | C |
| ATOM | 1616 | CG | PHE | A | 358 | −0.605 | 27.329 | −2.938 | 1.00 | 33.40 | C |
| ATOM | 1617 | CD1 | PHE | A | 358 | −1.138 | 27.503 | −4.207 | 1.00 | 33.96 | C |
| ATOM | 1618 | CE1 | PHE | A | 358 | −0.350 | 27.964 | −5.254 | 1.00 | 34.09 | C |
| ATOM | 1619 | CZ | PHE | A | 358 | 0.986 | 28.258 | −5.034 | 1.00 | 34.06 | C |
| ATOM | 1620 | CE2 | PHE | A | 358 | 1.526 | 28.092 | −3.775 | 1.00 | 33.93 | C |
| ATOM | 1621 | CD2 | PHE | A | 358 | 0.730 | 27.631 | −2.735 | 1.00 | 33.81 | C |
| ATOM | 1622 | C | PHE | A | 358 | −0.039 | 24.903 | −1.032 | 1.00 | 32.78 | C |
| ATOM | 1623 | O | PHE | A | 358 | 0.296 | 25.283 | 0.086 | 1.00 | 32.73 | O |
| ATOM | 1633 | N | LEU | A | 359 | 0.733 | 24.137 | −1.802 | 1.00 | 32.61 | N |
| ATOM | 1634 | CA | LEU | A | 359 | 2.042 | 23.641 | −1.379 | 1.00 | 32.50 | C |
| ATOM | 1635 | CB | LEU | A | 359 | 2.352 | 22.305 | −2.065 | 1.00 | 32.41 | C |
| ATOM | 1636 | CG | LEU | A | 359 | 1.500 | 21.103 | −1.641 | 1.00 | 32.41 | C |
| ATOM | 1637 | CD1 | LEU | A | 359 | 1.916 | 19.876 | −2.418 | 1.00 | 32.23 | C |
| ATOM | 1638 | CD2 | LEU | A | 359 | 1.606 | 20.837 | −0.141 | 1.00 | 32.63 | C |
| ATOM | 1639 | C | LEU | A | 359 | 3.170 | 24.635 | −1.662 | 1.00 | 32.41 | C |
| ATOM | 1640 | O | LEU | A | 359 | 2.997 | 25.586 | −2.420 | 1.00 | 32.43 | O |
| ATOM | 1652 | N | VAL | A | 360 | 4.326 | 24.394 | −1.045 | 1.00 | 32.14 | N |
| ATOM | 1653 | CA | VAL | A | 360 | 5.513 | 25.227 | −1.234 | 1.00 | 32.17 | C |
| ATOM | 1654 | CB | VAL | A | 360 | 6.612 | 24.898 | −0.170 | 1.00 | 32.13 | C |
| ATOM | 1655 | CG1 | VAL | A | 360 | 7.257 | 23.539 | −0.427 | 1.00 | 32.04 | C |
| ATOM | 1656 | CG2 | VAL | A | 360 | 7.658 | 25.995 | −0.108 | 1.00 | 31.93 | C |
| ATOM | 1657 | C | VAL | A | 360 | 6.079 | 25.117 | −2.658 | 1.00 | 32.12 | C |
| ATOM | 1658 | O | VAL | A | 360 | 6.755 | 26.028 | −3.135 | 1.00 | 32.06 | O |
| ATOM | 1668 | N | THR | A | 361 | 5.780 | 24.002 | −3.324 | 1.00 | 32.20 | N |
| ATOM | 1669 | CA | THR | A | 361 | 6.200 | 23.751 | −4.703 | 1.00 | 32.11 | C |
| ATOM | 1670 | CB | THR | A | 361 | 6.296 | 22.238 | −4.950 | 1.00 | 32.19 | C |
| ATOM | 1671 | OG1 | THR | A | 361 | 5.124 | 21.580 | −4.447 | 1.00 | 32.11 | O |
| ATOM | 1672 | CG2 | THR | A | 361 | 7.424 | 21.637 | −4.150 | 1.00 | 32.17 | C |
| ATOM | 1673 | C | THR | A | 361 | 5.284 | 24.379 | −5.764 | 1.00 | 32.17 | C |
| ATOM | 1674 | O | THR | A | 361 | 5.583 | 24.310 | −6.957 | 1.00 | 31.89 | O |
| ATOM | 1682 | N | GLY | A | 362 | 4.172 | 24.973 | −5.337 | 1.00 | 32.31 | N |
| ATOM | 1683 | CA | GLY | A | 362 | 3.347 | 25.787 | −6.222 | 1.00 | 32.41 | C |
| ATOM | 1684 | C | GLY | A | 362 | 2.003 | 25.211 | −6.634 | 1.00 | 32.46 | C |
| ATOM | 1685 | O | GLY | A | 362 | 1.171 | 25.935 | −7.178 | 1.00 | 32.54 | O |
| ATOM | 1689 | N | GLN | A | 363 | 1.777 | 23.927 | −6.370 | 1.00 | 32.56 | N |
| ATOM | 1690 | CA | GLN | A | 363 | 0.547 | 23.258 | −6.790 | 1.00 | 32.79 | C |
| ATOM | 1691 | CB | GLN | A | 363 | 0.812 | 21.790 | −7.103 | 1.00 | 32.86 | C |
| ATOM | 1692 | CG | GLN | A | 363 | 1.909 | 21.560 | −8.123 | 1.00 | 33.54 | C |
| ATOM | 1693 | CD | GLN | A | 363 | 3.300 | 21.598 | −7.519 | 1.00 | 34.24 | C |
| ATOM | 1694 | OE1 | GLN | A | 363 | 3.512 | 21.111 | −6.410 | 1.00 | 35.24 | O |
| ATOM | 1695 | NE2 | GLN | A | 363 | 4.247 | 22.184 | −8.242 | 1.00 | 34.95 | N |
| ATOM | 1696 | C | GLN | A | 363 | −0.535 | 23.348 | −5.719 | 1.00 | 32.83 | C |
| ATOM | 1697 | O | GLN | A | 363 | −0.268 | 23.724 | −4.580 | 1.00 | 32.41 | O |
| ATOM | 1706 | N | GLN | A | 364 | −1.759 | 22.991 | −6.100 | 1.00 | 32.98 | N |
| ATOM | 1707 | CA | GLN | A | 364 | −2.886 | 22.982 | −5.179 | 1.00 | 33.20 | C |
| ATOM | 1708 | CB | GLN | A | 364 | −3.951 | 23.981 | −5.628 | 1.00 | 33.20 | C |
| ATOM | 1709 | CG | GLN | A | 364 | −3.574 | 25.415 | −5.328 | 1.00 | 33.71 | C |
| ATOM | 1710 | CD | GLN | A | 364 | −4.765 | 26.324 | −5.131 | 1.00 | 34.23 | C |
| ATOM | 1711 | OE1 | GLN | A | 364 | −4.678 | 27.211 | −4.138 | 1.00 | 35.11 | O |
| ATOM | 1712 | NE2 | GLN | A | 364 | −5.753 | 26.236 | −5.872 | 1.00 | 33.26 | N |
| ATOM | 1713 | C | GLN | A | 364 | −3.478 | 21.592 | −5.098 | 1.00 | 33.35 | C |
| ATOM | 1714 | O | GLN | A | 364 | −4.246 | 21.194 | −5.966 | 1.00 | 33.60 | O |
| ATOM | 1723 | N | VAL | A | 365 | −3.111 | 20.850 | −4.059 | 1.00 | 33.56 | N |
| ATOM | 1724 | CA | VAL | A | 365 | −3.741 | 19.560 | −3.787 | 1.00 | 33.80 | C |
| ATOM | 1725 | CB | VAL | A | 365 | −2.866 | 18.670 | −2.869 | 1.00 | 33.82 | C |
| ATOM | 1726 | CG1 | VAL | A | 365 | −1.445 | 18.561 | −3.432 | 1.00 | 34.21 | C |
| ATOM | 1727 | CG2 | VAL | A | 365 | −2.825 | 19.202 | −1.440 | 1.00 | 34.08 | C |
| ATOM | 1728 | C | VAL | A | 365 | −5.115 | 19.815 | −3.167 | 1.00 | 33.83 | C |
| ATOM | 1729 | O | VAL | A | 365 | −5.245 | 20.623 | −2.258 | 1.00 | 33.87 | O |
| ATOM | 1739 | N | ASP | A | 366 | −6.143 | 19.151 | −3.681 | 1.00 | 33.96 | N |
| ATOM | 1740 | CA | ASP | A | 366 | −7.492 | 19.314 | −3.149 | 1.00 | 34.04 | C |
| ATOM | 1741 | CB | ASP | A | 366 | −8.522 | 18.599 | −4.033 | 1.00 | 34.21 | C |
| ATOM | 1742 | CG | ASP | A | 366 | −8.527 | 19.109 | −5.466 | 1.00 | 34.71 | C |
| ATOM | 1743 | OD1 | ASP | A | 366 | −9.479 | 18.782 | −6.209 | 1.00 | 34.96 | O |
| ATOM | 1744 | OD2 | ASP | A | 366 | −7.626 | 19.840 | −5.936 | 1.00 | 35.68 | O |
| ATOM | 1745 | C | ASP | A | 366 | −7.530 | 18.726 | −1.745 | 1.00 | 33.89 | C |
| ATOM | 1746 | O | ASP | A | 366 | −6.976 | 17.653 | −1.516 | 1.00 | 33.84 | O |
| ATOM | 1751 | N | TYR | A | 367 | −8.174 | 19.424 | −0.811 | 1.00 | 33.75 | N |
| ATOM | 1752 | CA | TYR | A | 367 | −8.304 | 18.936 | 0.563 | 1.00 | 33.80 | C |
| ATOM | 1753 | CB | TYR | A | 367 | −9.023 | 19.970 | 1.452 | 1.00 | 33.93 | C |
| ATOM | 1754 | CG | TYR | A | 367 | −9.343 | 19.462 | 2.846 | 1.00 | 34.89 | C |
| ATOM | 1755 | CD1 | TYR | A | 367 | −8.368 | 19.427 | 3.849 | 1.00 | 36.38 | C |
| ATOM | 1756 | CE1 | TYR | A | 367 | −8.661 | 18.939 | 5.125 | 1.00 | 37.01 | C |
| ATOM | 1757 | CZ | TYR | A | 367 | −9.944 | 18.479 | 5.398 | 1.00 | 38.19 | C |
| ATOM | 1758 | OH | TYR | A | 367 | −10.271 | 17.987 | 6.640 | 1.00 | 40.50 | O |
| ATOM | 1759 | CE2 | TYR | A | 367 | −10.920 | 18.503 | 4.422 | 1.00 | 37.48 | C |
| ATOM | 1760 | CD2 | TYR | A | 367 | −10.614 | 18.991 | 3.155 | 1.00 | 36.56 | C |
| ATOM | 1761 | C | TYR | A | 367 | −9.014 | 17.577 | 0.623 | 1.00 | 33.75 | C |
| ATOM | 1762 | O | TYR | A | 367 | −8.814 | 16.822 | 1.563 | 1.00 | 33.69 | O |
| ATOM | 1772 | N | SER | A | 368 | −9.837 | 17.276 | −0.382 | 1.00 | 33.85 | N |
| ATOM | 1773 | CA | SER | A | 368 | −10.509 | 15.974 | −0.508 | 1.00 | 33.98 | C |
| ATOM | 1774 | CB | SER | A | 368 | −11.280 | 15.904 | −1.834 | 1.00 | 34.00 | C |
| ATOM | 1775 | OG | SER | A | 368 | −11.625 | 17.198 | −2.309 | 1.00 | 34.49 | O |
| ATOM | 1776 | C | SER | A | 368 | −9.529 | 14.794 | −0.432 | 1.00 | 34.02 | C |
| ATOM | 1777 | O | SER | A | 368 | −9.789 | 13.792 | 0.235 | 1.00 | 33.77 | O |
| ATOM | 1783 | N | ILE | A | 369 | −8.412 | 14.936 | −1.141 | 1.00 | 34.21 | N |
| ATOM | 1784 | CA | ILE | A | 369 | −7.331 | 13.949 | −1.175 | 1.00 | 34.37 | C |
| ATOM | 1785 | CB | ILE | A | 369 | −6.213 | 14.451 | −2.142 | 1.00 | 34.54 | C |
| ATOM | 1786 | CG1 | ILE | A | 369 | −6.669 | 14.281 | −3.601 | 1.00 | 35.07 | C |
| ATOM | 1787 | CD1 | ILE | A | 369 | −6.411 | 15.511 | −4.464 | 1.00 | 35.66 | C |
| ATOM | 1788 | CG2 | ILE | A | 369 | −4.875 | 13.744 | −1.898 | 1.00 | 34.43 | C |
| ATOM | 1789 | C | ILE | A | 369 | −6.745 | 13.644 | 0.203 | 1.00 | 34.36 | C |
| ATOM | 1790 | O | ILE | A | 369 | −6.428 | 12.486 | 0.502 | 1.00 | 34.43 | O |
| ATOM | 1802 | N | ILE | A | 370 | −6.595 | 14.679 | 1.029 | 1.00 | 34.24 | N |
| ATOM | 1803 | CA | ILE | A | 370 | −6.007 | 14.523 | 2.360 | 1.00 | 34.08 | C |
| ATOM | 1804 | CB | ILE | A | 370 | −5.478 | 15.866 | 2.925 | 1.00 | 34.17 | C |
| ATOM | 1805 | CG1 | ILE | A | 370 | −4.742 | 16.687 | 1.854 | 1.00 | 33.90 | C |
| ATOM | 1806 | CD1 | ILE | A | 370 | −4.353 | 18.067 | 2.316 | 1.00 | 33.59 | C |
| ATOM | 1807 | CG2 | ILE | A | 370 | −4.533 | 15.601 | 4.095 | 1.00 | 34.30 | C |
| ATOM | 1808 | C | ILE | A | 370 | −7.015 | 13.909 | 3.328 | 1.00 | 33.94 | C |
| ATOM | 1809 | O | ILE | A | 370 | −6.656 | 13.066 | 4.138 | 1.00 | 34.11 | O |
| ATOM | 1821 | N | ALA | A | 371 | −8.274 | 14.322 | 3.225 | 1.00 | 33.84 | N |
| ATOM | 1822 | CA | ALA | A | 371 | −9.350 | 13.828 | 4.088 | 1.00 | 33.80 | C |
| ATOM | 1823 | CB | ALA | A | 371 | −10.659 | 14.505 | 3.718 | 1.00 | 33.80 | C |
| ATOM | 1824 | C | ALA | A | 371 | −9.530 | 12.312 | 4.034 | 1.00 | 33.78 | C |
| ATOM | 1825 | O | ALA | A | 371 | −9.734 | 11.674 | 5.063 | 1.00 | 33.95 | O |
| ATOM | 1831 | N | SER | A | 372 | −9.457 | 11.747 | 2.836 | 1.00 | 33.70 | N |
| ATOM | 1832 | CA | SER | A | 372 | −9.695 | 10.318 | 2.636 | 1.00 | 33.86 | C |
| ATOM | 1833 | CB | SER | A | 372 | −10.019 | 10.044 | 1.166 | 1.00 | 33.87 | C |
| ATOM | 1834 | OG | SER | A | 372 | −8.972 | 10.493 | 0.326 | 1.00 | 33.90 | O |
| ATOM | 1835 | C | SER | A | 372 | −8.536 | 9.414 | 3.067 | 1.00 | 33.97 | C |
| ATOM | 1836 | O | SER | A | 372 | −8.750 | 8.235 | 3.359 | 1.00 | 33.97 | O |
| ATOM | 1842 | N | GLN | A | 373 | −7.321 | 9.960 | 3.097 | 1.00 | 34.09 | N |
| ATOM | 1843 | CA | GLN | A | 373 | −6.111 | 9.173 | 3.346 | 1.00 | 34.20 | C |
| ATOM | 1844 | CB | GLN | A | 373 | −5.096 | 9.436 | 2.223 | 1.00 | 34.34 | C |
| ATOM | 1845 | CG | GLN | A | 373 | −4.472 | 8.176 | 1.610 | 1.00 | 35.16 | C |
| ATOM | 1846 | CD | GLN | A | 373 | −4.593 | 8.146 | 0.100 | 1.00 | 35.49 | C |
| ATOM | 1847 | OE1 | GLN | A | 373 | −4.000 | 8.979 | −0.593 | 1.00 | 36.05 | O |
| ATOM | 1848 | NE2 | GLN | A | 373 | −5.371 | 7.200 | −0.414 | 1.00 | 36.22 | N |
| ATOM | 1849 | C | GLN | A | 373 | −5.440 | 9.444 | 4.703 | 1.00 | 34.14 | C |
| ATOM | 1850 | O | GLN | A | 373 | −4.483 | 8.755 | 5.061 | 1.00 | 34.27 | O |
| ATOM | 1859 | N | ALA | A | 374 | −5.936 | 10.431 | 5.450 | 1.00 | 33.89 | N |
| ATOM | 1860 | CA | ALA | A | 374 | −5.306 | 10.860 | 6.699 | 1.00 | 33.61 | C |
| ATOM | 1861 | CB | ALA | A | 374 | −5.445 | 12.364 | 6.861 | 1.00 | 33.69 | C |
| ATOM | 1862 | C | ALA | A | 374 | −5.916 | 10.150 | 7.907 | 1.00 | 33.46 | C |
| ATOM | 1863 | O | ALA | A | 374 | −7.134 | 10.026 | 8.010 | 1.00 | 33.33 | O |
| ATOM | 1869 | N | GLY | A | 375 | −5.064 | 9.696 | 8.822 | 1.00 | 33.26 | N |
| ATOM | 1870 | CA | GLY | A | 375 | −5.514 | 9.101 | 10.069 | 1.00 | 33.10 | C |
| ATOM | 1871 | C | GLY | A | 375 | −5.955 | 10.140 | 11.089 | 1.00 | 32.86 | C |
| ATOM | 1872 | O | GLY | A | 375 | −6.176 | 11.297 | 10.749 | 1.00 | 32.93 | O |
| ATOM | 1876 | N | ALA | A | 376 | −6.065 | 9.727 | 12.347 | 1.00 | 32.67 | N |
| ATOM | 1877 | CA | ALA | A | 376 | −6.644 | 10.567 | 13.393 | 1.00 | 32.64 | C |
| ATOM | 1878 | CB | ALA | A | 376 | −6.963 | 9.738 | 14.623 | 1.00 | 32.62 | C |
| ATOM | 1879 | C | ALA | A | 376 | −5.756 | 11.743 | 13.774 | 1.00 | 32.62 | C |
| ATOM | 1880 | O | ALA | A | 376 | −6.232 | 12.875 | 13.828 | 1.00 | 32.68 | O |
| ATOM | 1886 | N | THR | A | 377 | −4.480 | 11.474 | 14.052 | 1.00 | 32.45 | N |
| ATOM | 1887 | CA | THR | A | 377 | −3.543 | 12.517 | 14.477 | 1.00 | 32.26 | C |
| ATOM | 1888 | CB | THR | A | 377 | −2.125 | 11.945 | 14.734 | 1.00 | 32.25 | C |
| ATOM | 1889 | OG1 | THR | A | 377 | −2.180 | 10.870 | 15.678 | 1.00 | 32.16 | O |
| ATOM | 1890 | CG2 | THR | A | 377 | −1.242 | 12.966 | 15.441 | 1.00 | 32.64 | C |
| ATOM | 1891 | C | THR | A | 377 | −3.446 | 13.628 | 13.453 | 1.00 | 32.17 | C |
| ATOM | 1892 | O | THR | A | 377 | −3.510 | 14.804 | 13.801 | 1.00 | 32.06 | O |
| ATOM | 1900 | N | LEU | A | 378 | −3.282 | 13.248 | 12.194 | 1.00 | 32.27 | N |
| ATOM | 1901 | CA | LEU | A | 378 | −3.087 | 14.211 | 11.116 | 1.00 | 32.63 | C |
| ATOM | 1902 | CB | LEU | A | 378 | −2.616 | 13.503 | 9.843 | 1.00 | 32.60 | C |
| ATOM | 1903 | CG | LEU | A | 378 | −2.487 | 14.297 | 8.547 | 1.00 | 32.45 | C |
| ATOM | 1904 | CD1 | LEU | A | 378 | −1.401 | 15.347 | 8.650 | 1.00 | 32.47 | C |
| ATOM | 1905 | CD2 | LEU | A | 378 | −2.188 | 13.335 | 7.432 | 1.00 | 32.88 | C |
| ATOM | 1906 | C | LEU | A | 378 | −4.348 | 14.998 | 10.831 | 1.00 | 32.93 | C |
| ATOM | 1907 | O | LEU | A | 378 | −4.270 | 16.142 | 10.410 | 1.00 | 32.91 | O |
| ATOM | 1919 | N | ASN | A | 379 | −5.502 | 14.374 | 11.041 | 1.00 | 33.56 | N |
| ATOM | 1920 | CA | ASN | A | 379 | −6.786 | 15.054 | 10.884 | 1.00 | 34.00 | C |
| ATOM | 1921 | CB | ASN | A | 379 | −7.948 | 14.059 | 10.938 | 1.00 | 34.08 | C |
| ATOM | 1922 | CG | ASN | A | 379 | −8.555 | 13.807 | 9.578 | 1.00 | 35.10 | C |
| ATOM | 1923 | OD1 | ASN | A | 379 | −9.189 | 14.834 | 9.022 | 1.00 | 37.09 | O |
| ATOM | 1924 | ND2 | ASN | A | 379 | −8.454 | 12.702 | 9.024 | 1.00 | 35.56 | N |
| ATOM | 1925 | C | ASN | A | 379 | −6.974 | 16.139 | 11.941 | 1.00 | 34.04 | C |
| ATOM | 1926 | O | ASN | A | 379 | −7.458 | 17.226 | 11.638 | 1.00 | 33.86 | O |
| ATOM | 1933 | N | ASN | A | 380 | −6.584 | 15.830 | 13.178 | 1.00 | 34.33 | N |
| ATOM | 1934 | CA | ASN | A | 380 | −6.598 | 16.800 | 14.271 | 1.00 | 34.68 | C |
| ATOM | 1935 | CB | ASN | A | 380 | −6.188 | 16.153 | 15.605 | 1.00 | 34.62 | C |
| ATOM | 1936 | CG | ASN | A | 380 | −7.263 | 15.241 | 16.175 | 1.00 | 35.17 | C |
| ATOM | 1937 | OD1 | ASN | A | 380 | −8.429 | 15.623 | 16.265 | 1.00 | 36.08 | O |
| ATOM | 1938 | ND2 | ASN | A | 380 | −6.873 | 14.025 | 16.568 | 1.00 | 35.65 | N |
| ATOM | 1939 | C | ASN | A | 380 | −5.677 | 17.976 | 13.975 | 1.00 | 34.86 | C |
| ATOM | 1940 | O | ASN | A | 380 | −5.983 | 19.100 | 14.349 | 1.00 | 35.04 | O |
| ATOM | 1947 | N | LEU | A | 381 | −4.563 | 17.704 | 13.297 | 1.00 | 35.08 | N |
| ATOM | 1948 | CA | LEU | A | 381 | −3.554 | 18.720 | 12.981 | 1.00 | 35.42 | C |
| ATOM | 1949 | CB | LEU | A | 381 | −2.219 | 18.061 | 12.628 | 1.00 | 35.50 | C |
| ATOM | 1950 | CG | LEU | A | 381 | −1.230 | 17.804 | 13.754 | 1.00 | 35.83 | C |
| ATOM | 1951 | CD1 | LEU | A | 381 | −0.150 | 16.866 | 13.243 | 1.00 | 37.15 | C |
| ATOM | 1952 | CD2 | LEU | A | 381 | −0.621 | 19.109 | 14.249 | 1.00 | 36.34 | C |
| ATOM | 1953 | C | LEU | A | 381 | −3.951 | 19.587 | 11.804 | 1.00 | 35.60 | C |
| ATOM | 1954 | O | LEU | A | 381 | −3.771 | 20.792 | 11.837 | 1.00 | 35.44 | O |
| ATOM | 1966 | N | MET | A | 382 | −4.446 | 18.948 | 10.748 | 1.00 | 36.32 | N |
| ATOM | 1967 | CA | MET | A | 382 | −4.815 | 19.630 | 9.507 | 1.00 | 36.73 | C |
| ATOM | 1968 | CB | MET | A | 382 | −5.170 | 18.616 | 8.403 | 1.00 | 37.03 | C |
| ATOM | 1969 | CG | MET | A | 382 | −4.049 | 18.370 | 7.386 | 1.00 | 38.21 | C |
| ATOM | 1970 | SD | MET | A | 382 | −3.965 | 19.676 | 6.089 | 1.00 | 41.87 | S |
| ATOM | 1971 | CE | MET | A | 382 | −2.387 | 20.427 | 6.432 | 1.00 | 40.39 | C |
| ATOM | 1972 | C | MET | A | 382 | −6.001 | 20.550 | 9.767 | 1.00 | 36.67 | C |
| ATOM | 1973 | O | MET | A | 382 | −6.090 | 21.636 | 9.189 | 1.00 | 36.42 | O |
| ATOM | 1983 | N | SER | A | 383 | −6.893 | 20.104 | 10.651 | 1.00 | 36.53 | N |
| ATOM | 1984 | CA | SER | A | 383 | −8.102 | 20.846 | 10.975 | 1.00 | 36.59 | C |
| ATOM | 1985 | CB | SER | A | 383 | −9.166 | 19.930 | 11.597 | 1.00 | 36.67 | C |
| ATOM | 1986 | OG | SER | A | 383 | −8.946 | 19.729 | 12.985 | 1.00 | 37.42 | O |
| ATOM | 1987 | C | SER | A | 383 | −7.813 | 22.029 | 11.894 | 1.00 | 36.27 | C |
| ATOM | 1988 | O | SER | A | 383 | −8.365 | 23.104 | 11.691 | 1.00 | 36.52 | O |
| ATOM | 1994 | N | HIS | A | 384 | −6.955 | 21.845 | 12.896 | 1.00 | 35.86 | N |
| ATOM | 1995 | CA | HIS | A | 384 | −6.545 | 22.974 | 13.743 | 1.00 | 35.64 | C |
| ATOM | 1996 | CB | HIS | A | 384 | −5.637 | 22.528 | 14.898 | 1.00 | 35.66 | C |
| ATOM | 1997 | CG | HIS | A | 384 | −6.295 | 21.571 | 15.846 | 1.00 | 36.74 | C |
| ATOM | 1998 | ND1 | HIS | A | 384 | −5.582 | 20.799 | 16.740 | 1.00 | 37.77 | N |
| ATOM | 1999 | CE1 | HIS | A | 384 | −6.418 | 20.039 | 17.428 | 1.00 | 37.38 | C |
| ATOM | 2000 | NE2 | HIS | A | 384 | −7.646 | 20.285 | 17.010 | 1.00 | 37.06 | N |
| ATOM | 2001 | CD2 | HIS | A | 384 | −7.598 | 21.240 | 16.021 | 1.00 | 36.93 | C |
| ATOM | 2002 | C | HIS | A | 384 | −5.849 | 24.027 | 12.886 | 1.00 | 35.20 | C |
| ATOM | 2003 | O | HIS | A | 384 | −6.069 | 25.217 | 13.073 | 1.00 | 35.15 | O |
| ATOM | 2012 | N | ALA | A | 385 | −5.037 | 23.571 | 11.931 | 1.00 | 34.96 | N |
| ATOM | 2013 | CA | ALA | A | 385 | −4.356 | 24.450 | 10.986 | 1.00 | 34.90 | C |
| ATOM | 2014 | CB | ALA | A | 385 | −3.433 | 23.653 | 10.095 | 1.00 | 34.70 | C |
| ATOM | 2015 | C | ALA | A | 385 | −5.323 | 25.256 | 10.131 | 1.00 | 35.19 | C |
| ATOM | 2016 | O | ALA | A | 385 | −5.062 | 26.418 | 9.849 | 1.00 | 35.14 | O |
| ATOM | 2022 | N | GLN | A | 386 | −6.435 | 24.642 | 9.730 | 1.00 | 35.67 | N |
| ATOM | 2023 | CA | GLN | A | 386 | −7.397 | 25.283 | 8.828 | 1.00 | 36.06 | C |
| ATOM | 2024 | CB | GLN | A | 386 | −8.306 | 24.251 | 8.145 | 1.00 | 36.24 | C |
| ATOM | 2025 | CG | GLN | A | 386 | −8.358 | 24.392 | 6.621 | 1.00 | 37.29 | C |
| ATOM | 2026 | CD | GLN | A | 386 | −6.987 | 24.230 | 5.979 | 1.00 | 39.41 | C |
| ATOM | 2027 | OE1 | GLN | A | 386 | −6.278 | 23.256 | 6.265 | 1.00 | 40.61 | O |
| ATOM | 2028 | NE2 | GLN | A | 386 | −6.599 | 25.192 | 5.129 | 1.00 | 40.28 | N |
| ATOM | 2029 | C | GLN | A | 386 | −8.247 | 26.340 | 9.523 | 1.00 | 36.15 | C |
| ATOM | 2030 | O | GLN | A | 386 | −8.578 | 27.351 | 8.907 | 1.00 | 36.33 | O |
| ATOM | 2039 | N | GLU | A | 387 | −8.594 | 26.110 | 10.792 | 1.00 | 36.03 | N |
| ATOM | 2040 | CA | GLU | A | 387 | −9.329 | 27.100 | 11.581 | 1.00 | 35.96 | C |
| ATOM | 2041 | CB | GLU | A | 387 | −9.845 | 26.508 | 12.901 | 1.00 | 36.12 | C |
| ATOM | 2042 | CG | GLU | A | 387 | −10.708 | 25.249 | 12.780 | 1.00 | 36.83 | C |
| ATOM | 2043 | CD | GLU | A | 387 | −11.928 | 25.410 | 11.876 | 1.00 | 38.35 | C |
| ATOM | 2044 | OE1 | GLU | A | 387 | −12.765 | 26.314 | 12.122 | 1.00 | 39.69 | O |
| ATOM | 2045 | OE2 | GLU | A | 387 | −12.061 | 24.616 | 10.920 | 1.00 | 38.32 | O |
| ATOM | 2046 | C | GLU | A | 387 | −8.433 | 28.295 | 11.878 | 1.00 | 35.69 | C |
| ATOM | 2047 | O | GLU | A | 387 | −8.916 | 29.401 | 12.105 | 1.00 | 36.05 | O |
| ATOM | 2054 | N | LEU | A | 388 | −7.125 | 28.065 | 11.876 | 1.00 | 35.32 | N |
| ATOM | 2055 | CA | LEU | A | 388 | −6.137 | 29.123 | 12.092 | 1.00 | 34.88 | C |
| ATOM | 2056 | CB | LEU | A | 388 | −4.791 | 28.500 | 12.480 | 1.00 | 34.87 | C |
| ATOM | 2057 | CG | LEU | A | 388 | −3.788 | 29.263 | 13.350 | 1.00 | 34.41 | C |
| ATOM | 2058 | CD1 | LEU | A | 388 | −2.382 | 28.927 | 12.914 | 1.00 | 34.37 | C |
| ATOM | 2059 | CD2 | LEU | A | 388 | −3.982 | 30.759 | 13.317 | 1.00 | 34.53 | C |
| ATOM | 2060 | C | LEU | A | 388 | −5.981 | 29.966 | 10.823 | 1.00 | 34.58 | C |
| ATOM | 2061 | O | LEU | A | 388 | −5.921 | 31.193 | 10.886 | 1.00 | 34.02 | O |
| ATOM | 2073 | N | VAL | A | 389 | −5.915 | 29.284 | 9.679 | 1.00 | 34.42 | N |
| ATOM | 2074 | CA | VAL | A | 389 | −5.860 | 29.926 | 8.365 | 1.00 | 34.23 | C |
| ATOM | 2075 | CB | VAL | A | 389 | −5.813 | 28.870 | 7.202 | 1.00 | 34.23 | C |
| ATOM | 2076 | CG1 | VAL | A | 389 | −6.074 | 29.513 | 5.842 | 1.00 | 33.63 | C |
| ATOM | 2077 | CG2 | VAL | A | 389 | −4.474 | 28.140 | 7.183 | 1.00 | 34.32 | C |
| ATOM | 2078 | C | VAL | A | 389 | −7.075 | 30.822 | 8.170 | 1.00 | 34.10 | C |
| ATOM | 2079 | O | VAL | A | 389 | −6.947 | 31.931 | 7.665 | 1.00 | 34.30 | O |
| ATOM | 2089 | N | ALA | A | 390 | −8.242 | 30.336 | 8.584 | 1.00 | 33.97 | N |
| ATOM | 2090 | CA | ALA | A | 390 | −9.509 | 31.031 | 8.370 | 1.00 | 34.01 | C |
| ATOM | 2091 | CB | ALA | A | 390 | −10.675 | 30.056 | 8.527 | 1.00 | 33.92 | C |
| ATOM | 2092 | C | ALA | A | 390 | −9.674 | 32.222 | 9.316 | 1.00 | 34.14 | C |
| ATOM | 2093 | O | ALA | A | 390 | −10.369 | 33.186 | 8.993 | 1.00 | 34.11 | O |
| ATOM | 2099 | N | LYS | A | 391 | −9.038 | 32.146 | 10.482 | 1.00 | 34.37 | N |
| ATOM | 2100 | CA | LYS | A | 391 | −9.016 | 33.261 | 11.424 | 1.00 | 34.68 | C |
| ATOM | 2101 | CB | LYS | A | 391 | −8.430 | 32.820 | 12.774 | 1.00 | 34.82 | C |
| ATOM | 2102 | CG | LYS | A | 391 | −9.136 | 33.404 | 14.007 | 1.00 | 35.74 | C |
| ATOM | 2103 | CD | LYS | A | 391 | −9.520 | 32.302 | 15.024 | 1.00 | 36.81 | C |
| ATOM | 2104 | CE | LYS | A | 391 | −10.838 | 31.609 | 14.679 | 1.00 | 36.91 | C |
| ATOM | 2105 | NZ | LYS | A | 391 | −10.727 | 30.130 | 14.854 | 1.00 | 37.71 | N |
| ATOM | 2106 | C | LYS | A | 391 | −8.185 | 34.398 | 10.830 | 1.00 | 34.75 | C |
| ATOM | 2107 | O | LYS | A | 391 | −8.597 | 35.557 | 10.849 | 1.00 | 34.88 | O |
| ATOM | 2121 | N | LEU | A | 392 | −7.019 | 34.038 | 10.294 | 1.00 | 34.71 | N |
| ATOM | 2122 | CA | LEU | A | 392 | −6.111 | 34.983 | 9.651 | 1.00 | 34.53 | C |
| ATOM | 2123 | CB | LEU | A | 392 | −4.748 | 34.330 | 9.376 | 1.00 | 34.46 | C |
| ATOM | 2124 | CG | LEU | A | 392 | −3.516 | 34.607 | 10.257 | 1.00 | 34.48 | C |
| ATOM | 2125 | CD1 | LEU | A | 392 | −3.795 | 35.339 | 11.565 | 1.00 | 34.91 | C |
| ATOM | 2126 | CD2 | LEU | A | 392 | −2.808 | 33.299 | 10.540 | 1.00 | 34.38 | C |
| ATOM | 2127 | C | LEU | A | 392 | −6.684 | 35.488 | 8.339 | 1.00 | 34.60 | C |
| ATOM | 2128 | O | LEU | A | 392 | −6.404 | 36.613 | 7.943 | 1.00 | 34.96 | O |
| ATOM | 2140 | N | ARG | A | 393 | −7.483 | 34.671 | 7.659 | 1.00 | 34.59 | N |
| ATOM | 2141 | CA | ARG | A | 393 | −8.050 | 35.079 | 6.376 | 1.00 | 34.71 | C |
| ATOM | 2142 | CB | ARG | A | 393 | −8.764 | 33.911 | 5.678 | 1.00 | 34.77 | C |
| ATOM | 2143 | CG | ARG | A | 393 | −8.670 | 33.925 | 4.146 | 1.00 | 35.35 | C |
| ATOM | 2144 | CD | ARG | A | 393 | −8.054 | 32.655 | 3.542 | 1.00 | 36.10 | C |
| ATOM | 2145 | NE | ARG | A | 393 | −8.962 | 31.510 | 3.635 | 1.00 | 36.52 | N |
| ATOM | 2146 | CZ | ARG | A | 393 | −8.634 | 30.242 | 3.368 | 1.00 | 36.68 | C |
| ATOM | 2147 | NH1 | ARG | A | 393 | −7.405 | 29.919 | 2.972 | 1.00 | 36.25 | N |
| ATOM | 2148 | NH2 | ARG | A | 393 | −9.552 | 29.285 | 3.493 | 1.00 | 36.62 | N |
| ATOM | 2149 | C | ARG | A | 393 | −9.012 | 36.235 | 6.618 | 1.00 | 34.60 | C |
| ATOM | 2150 | O | ARG | A | 393 | −9.026 | 37.215 | 5.868 | 1.00 | 34.50 | O |
| ATOM | 2164 | N | SER | A | 394 | −9.782 | 36.115 | 7.697 | 1.00 | 34.66 | N |
| ATOM | 2165 | CA | SER | A | 394 | −10.786 | 37.105 | 8.081 | 1.00 | 34.84 | C |
| ATOM | 2166 | CB | SER | A | 394 | −11.803 | 36.477 | 9.032 | 1.00 | 34.84 | C |
| ATOM | 2167 | OG | SER | A | 394 | −11.148 | 35.935 | 10.168 | 1.00 | 35.49 | O |
| ATOM | 2168 | C | SER | A | 394 | −10.194 | 38.352 | 8.734 | 1.00 | 34.77 | C |
| ATOM | 2169 | O | SER | A | 394 | −10.910 | 39.324 | 8.939 | 1.00 | 35.10 | O |
| ATOM | 2175 | N | LEU | A | 395 | −8.905 | 38.316 | 9.071 | 1.00 | 34.79 | N |
| ATOM | 2176 | CA | LEU | A | 395 | −8.170 | 39.493 | 9.561 | 1.00 | 34.82 | C |
| ATOM | 2177 | CB | LEU | A | 395 | −7.116 | 39.067 | 10.599 | 1.00 | 34.93 | C |
| ATOM | 2178 | CG | LEU | A | 395 | −7.469 | 38.936 | 12.079 | 1.00 | 34.71 | C |
| ATOM | 2179 | CD1 | LEU | A | 395 | −8.959 | 38.698 | 12.302 | 1.00 | 35.03 | C |
| ATOM | 2180 | CD2 | LEU | A | 395 | −6.632 | 37.818 | 12.704 | 1.00 | 34.11 | C |
| ATOM | 2181 | C | LEU | A | 395 | −7.441 | 40.245 | 8.443 | 1.00 | 34.81 | C |
| ATOM | 2182 | O | LEU | A | 395 | −6.627 | 41.122 | 8.726 | 1.00 | 34.82 | O |
| ATOM | 2194 | N | GLN | A | 396 | −7.723 | 39.905 | 7.185 | 1.00 | 34.89 | N |
| ATOM | 2195 | CA | GLN | A | 396 | −6.929 | 40.380 | 6.046 | 1.00 | 34.84 | C |
| ATOM | 2196 | CB | GLN | A | 396 | −7.413 | 41.741 | 5.555 | 1.00 | 34.96 | C |
| ATOM | 2197 | CG | GLN | A | 396 | −8.792 | 41.712 | 4.927 | 1.00 | 35.71 | C |
| ATOM | 2198 | CD | GLN | A | 396 | −9.868 | 42.050 | 5.926 | 1.00 | 36.74 | C |
| ATOM | 2199 | OE1 | GLN | A | 396 | −10.109 | 41.284 | 6.867 | 1.00 | 37.26 | O |
| ATOM | 2200 | NE2 | GLN | A | 396 | −10.504 | 43.205 | 5.748 | 1.00 | 36.40 | N |
| ATOM | 2201 | C | GLN | A | 396 | −5.440 | 40.430 | 6.396 | 1.00 | 34.49 | C |
| ATOM | 2202 | O | GLN | A | 396 | −4.780 | 41.460 | 6.250 | 1.00 | 34.51 | O |
| ATOM | 2211 | N | PHE | A | 397 | −4.945 | 39.298 | 6.883 | 1.00 | 34.03 | N |
| ATOM | 2212 | CA | PHE | A | 397 | −3.534 | 39.101 | 7.190 | 1.00 | 33.67 | C |
| ATOM | 2213 | CB | PHE | A | 397 | −3.337 | 37.641 | 7.608 | 1.00 | 33.47 | C |
| ATOM | 2214 | CG | PHE | A | 397 | −2.000 | 37.331 | 8.215 | 1.00 | 33.19 | C |
| ATOM | 2215 | CD1 | PHE | A | 397 | −1.432 | 38.159 | 9.170 | 1.00 | 32.83 | C |
| ATOM | 2216 | CE1 | PHE | A | 397 | −0.211 | 37.850 | 9.732 | 1.00 | 32.62 | C |
| ATOM | 2217 | CZ | PHE | A | 397 | 0.448 | 36.695 | 9.358 | 1.00 | 32.59 | C |
| ATOM | 2218 | CE2 | PHE | A | 397 | −0.112 | 35.855 | 8.422 | 1.00 | 32.92 | C |
| ATOM | 2219 | CD2 | PHE | A | 397 | −1.330 | 36.169 | 7.859 | 1.00 | 33.03 | C |
| ATOM | 2220 | C | PHE | A | 397 | −2.696 | 39.437 | 5.956 | 1.00 | 33.58 | C |
| ATOM | 2221 | O | PHE | A | 397 | −2.939 | 38.897 | 4.876 | 1.00 | 33.65 | O |
| ATOM | 2231 | N | ASP | A | 398 | −1.735 | 40.343 | 6.107 | 1.00 | 33.35 | N |
| ATOM | 2232 | CA | ASP | A | 398 | −0.904 | 40.768 | 4.982 | 1.00 | 33.41 | C |
| ATOM | 2233 | CB | ASP | A | 398 | −1.181 | 42.239 | 4.618 | 1.00 | 33.37 | C |
| ATOM | 2234 | CG | ASP | A | 398 | −0.824 | 43.207 | 5.728 | 1.00 | 33.14 | C |
| ATOM | 2235 | OD1 | ASP | A | 398 | −1.559 | 44.198 | 5.914 | 1.00 | 32.83 | O |
| ATOM | 2236 | OD2 | ASP | A | 398 | 0.173 | 43.079 | 6.455 | 1.00 | 33.24 | O |
| ATOM | 2237 | C | ASP | A | 398 | 0.583 | 40.502 | 5.251 | 1.00 | 33.44 | C |
| ATOM | 2238 | O | ASP | A | 398 | 0.946 | 39.966 | 6.302 | 1.00 | 33.44 | O |
| ATOM | 2243 | N | GLN | A | 399 | 1.431 | 40.875 | 4.295 | 1.00 | 33.34 | N |
| ATOM | 2244 | CA | GLN | A | 399 | 2.854 | 40.555 | 4.350 | 1.00 | 33.38 | C |
| ATOM | 2245 | CB | GLN | A | 399 | 3.501 | 40.777 | 2.987 | 1.00 | 33.34 | C |
| ATOM | 2246 | CG | GLN | A | 399 | 4.830 | 40.051 | 2.830 | 1.00 | 33.63 | C |
| ATOM | 2247 | CD | GLN | A | 399 | 5.540 | 40.388 | 1.538 | 1.00 | 33.48 | C |
| ATOM | 2248 | OE1 | GLN | A | 399 | 5.336 | 41.459 | 0.967 | 1.00 | 34.38 | O |
| ATOM | 2249 | NE2 | GLN | A | 399 | 6.381 | 39.479 | 1.078 | 1.00 | 33.37 | N |
| ATOM | 2250 | C | GLN | A | 399 | 3.629 | 41.338 | 5.415 | 1.00 | 33.38 | C |
| ATOM | 2251 | O | GLN | A | 399 | 4.607 | 40.828 | 5.961 | 1.00 | 33.40 | O |
| ATOM | 2260 | N | ARG | A | 400 | 3.204 | 42.567 | 5.697 | 1.00 | 33.19 | N |
| ATOM | 2261 | CA | ARG | A | 400 | 3.834 | 43.384 | 6.734 | 1.00 | 33.12 | C |
| ATOM | 2262 | CB | ARG | A | 400 | 3.236 | 44.795 | 6.760 | 1.00 | 33.18 | C |
| ATOM | 2263 | CG | ARG | A | 400 | 3.771 | 45.746 | 5.697 | 1.00 | 33.28 | C |
| ATOM | 2264 | CD | ARG | A | 400 | 3.441 | 45.372 | 4.257 | 1.00 | 33.87 | C |
| ATOM | 2265 | NE | ARG | A | 400 | 2.012 | 45.152 | 4.024 | 1.00 | 34.41 | N |
| ATOM | 2266 | CZ | ARG | A | 400 | 1.437 | 45.077 | 2.823 | 1.00 | 35.12 | C |
| ATOM | 2267 | NH1 | ARG | A | 400 | 2.152 | 45.210 | 1.707 | 1.00 | 35.16 | N |
| ATOM | 2268 | NH2 | ARG | A | 400 | 0.128 | 44.870 | 2.737 | 1.00 | 35.41 | N |
| ATOM | 2269 | C | ARG | A | 400 | 3.637 | 42.739 | 8.101 | 1.00 | 33.14 | C |
| ATOM | 2270 | O | ARG | A | 400 | 4.571 | 42.637 | 8.902 | 1.00 | 33.34 | O |
| ATOM | 2284 | N | GLU | A | 401 | 2.405 | 42.317 | 8.358 | 1.00 | 32.93 | N |
| ATOM | 2285 | CA | GLU | A | 401 | 2.043 | 41.662 | 9.605 | 1.00 | 32.83 | C |
| ATOM | 2286 | CB | GLU | A | 401 | 0.522 | 41.490 | 9.678 | 1.00 | 32.92 | C |
| ATOM | 2287 | CG | GLU | A | 401 | −0.256 | 42.802 | 9.720 | 1.00 | 32.72 | C |
| ATOM | 2288 | CD | GLU | A | 401 | −1.709 | 42.643 | 9.327 | 1.00 | 32.50 | C |
| ATOM | 2289 | OE1 | GLU | A | 401 | −2.163 | 41.499 | 9.150 | 1.00 | 32.82 | O |
| ATOM | 2290 | OE2 | GLU | A | 401 | −2.409 | 43.664 | 9.205 | 1.00 | 33.15 | O |
| ATOM | 2291 | C | GLU | A | 401 | 2.715 | 40.294 | 9.727 | 1.00 | 32.66 | C |
| ATOM | 2292 | O | GLU | A | 401 | 3.110 | 39.887 | 10.810 | 1.00 | 32.29 | O |
| ATOM | 2299 | N | PHE | A | 402 | 2.847 | 39.599 | 8.601 | 1.00 | 32.76 | N |
| ATOM | 2300 | CA | PHE | A | 402 | 3.440 | 38.260 | 8.566 | 1.00 | 32.72 | C |
| ATOM | 2301 | CB | PHE | A | 402 | 3.226 | 37.644 | 7.177 | 1.00 | 32.72 | C |
| ATOM | 2302 | CG | PHE | A | 402 | 4.169 | 36.520 | 6.848 | 1.00 | 32.34 | C |
| ATOM | 2303 | CD1 | PHE | A | 402 | 3.993 | 35.265 | 7.414 | 1.00 | 32.74 | C |
| ATOM | 2304 | CE1 | PHE | A | 402 | 4.856 | 34.214 | 7.101 | 1.00 | 33.43 | C |
| ATOM | 2305 | CZ | PHE | A | 402 | 5.908 | 34.421 | 6.212 | 1.00 | 33.25 | C |
| ATOM | 2306 | CE2 | PHE | A | 402 | 6.088 | 35.676 | 5.639 | 1.00 | 32.90 | C |
| ATOM | 2307 | CD2 | PHE | A | 402 | 5.220 | 36.716 | 5.958 | 1.00 | 32.18 | C |
| ATOM | 2308 | C | PHE | A | 402 | 4.933 | 38.261 | 8.940 | 1.00 | 32.65 | C |
| ATOM | 2309 | O | PHE | A | 402 | 5.369 | 37.473 | 9.778 | 1.00 | 32.26 | O |
| ATOM | 2319 | N | VAL | A | 403 | 5.700 | 39.153 | 8.318 | 1.00 | 32.50 | N |
| ATOM | 2320 | CA | VAL | A | 403 | 7.133 | 39.256 | 8.572 | 1.00 | 32.59 | C |
| ATOM | 2321 | CB | VAL | A | 403 | 7.819 | 40.232 | 7.574 | 1.00 | 32.76 | C |
| ATOM | 2322 | CG1 | VAL | A | 403 | 9.227 | 40.501 | 7.987 | 1.00 | 33.35 | C |
| ATOM | 2323 | CG2 | VAL | A | 403 | 7.808 | 39.666 | 6.156 | 1.00 | 32.98 | C |
| ATOM | 2324 | C | VAL | A | 403 | 7.416 | 39.684 | 10.025 | 1.00 | 32.33 | C |
| ATOM | 2325 | O | VAL | A | 403 | 8.432 | 39.294 | 10.596 | 1.00 | 32.22 | O |
| ATOM | 2335 | N | CYS | A | 404 | 6.525 | 40.482 | 10.612 | 1.00 | 32.07 | N |
| ATOM | 2336 | CA | CYS | A | 404 | 6.619 | 40.825 | 12.034 | 1.00 | 31.98 | C |
| ATOM | 2337 | CB | CYS | A | 404 | 5.576 | 41.868 | 12.437 | 1.00 | 31.95 | C |
| ATOM | 2338 | SG | CYS | A | 404 | 6.006 | 43.553 | 11.984 | 1.00 | 32.73 | S |
| ATOM | 2339 | C | CYS | A | 404 | 6.434 | 39.587 | 12.896 | 1.00 | 31.68 | C |
| ATOM | 2340 | O | CYS | A | 404 | 7.240 | 39.325 | 13.789 | 1.00 | 31.32 | O |
| ATOM | 2346 | N | LEU | A | 405 | 5.376 | 38.825 | 12.627 | 1.00 | 31.53 | N |
| ATOM | 2347 | CA | LEU | A | 405 | 5.103 | 37.619 | 13.404 | 1.00 | 31.60 | C |
| ATOM | 2348 | CB | LEU | A | 405 | 3.796 | 36.952 | 12.959 | 1.00 | 31.77 | C |
| ATOM | 2349 | CG | LEU | A | 405 | 2.460 | 37.607 | 13.353 | 1.00 | 32.40 | C |
| ATOM | 2350 | CD1 | LEU | A | 405 | 1.399 | 36.540 | 13.614 | 1.00 | 33.60 | C |
| ATOM | 2351 | CD2 | LEU | A | 405 | 2.572 | 38.535 | 14.558 | 1.00 | 32.88 | C |
| ATOM | 2352 | C | LEU | A | 405 | 6.265 | 36.636 | 13.326 | 1.00 | 31.30 | C |
| ATOM | 2353 | O | LEU | A | 405 | 6.578 | 35.983 | 14.310 | 1.00 | 30.88 | O |
| ATOM | 2365 | N | LYS | A | 406 | 6.915 | 36.566 | 12.166 | 1.00 | 31.55 | N |
| ATOM | 2366 | CA | LYS | A | 406 | 8.083 | 35.705 | 11.973 | 1.00 | 31.91 | C |
| ATOM | 2367 | CB | LYS | A | 406 | 8.575 | 35.779 | 10.531 | 1.00 | 32.21 | C |
| ATOM | 2368 | CG | LYS | A | 406 | 7.904 | 34.806 | 9.568 | 1.00 | 32.94 | C |
| ATOM | 2369 | CD | LYS | A | 406 | 8.875 | 34.340 | 8.478 | 1.00 | 33.06 | C |
| ATOM | 2370 | CE | LYS | A | 406 | 9.080 | 35.379 | 7.408 | 1.00 | 33.84 | C |
| ATOM | 2371 | NZ | LYS | A | 406 | 9.476 | 34.748 | 6.105 | 1.00 | 35.59 | N |
| ATOM | 2372 | C | LYS | A | 406 | 9.234 | 36.084 | 12.904 | 1.00 | 31.90 | C |
| ATOM | 2373 | O | LYS | A | 406 | 9.869 | 35.207 | 13.498 | 1.00 | 31.79 | O |
| ATOM | 2387 | N | PHE | A | 407 | 9.499 | 37.385 | 13.018 | 1.00 | 31.82 | N |
| ATOM | 2388 | CA | PHE | A | 407 | 10.566 | 37.888 | 13.883 | 1.00 | 31.91 | C |
| ATOM | 2389 | CB | PHE | A | 407 | 10.943 | 39.327 | 13.513 | 1.00 | 32.04 | C |
| ATOM | 2390 | CG | PHE | A | 407 | 11.979 | 39.419 | 12.425 | 1.00 | 32.81 | C |
| ATOM | 2391 | CD1 | PHE | A | 407 | 11.618 | 39.324 | 11.085 | 1.00 | 33.43 | C |
| ATOM | 2392 | CE1 | PHE | A | 407 | 12.575 | 39.401 | 10.080 | 1.00 | 33.43 | C |
| ATOM | 2393 | CZ | PHE | A | 407 | 13.896 | 39.568 | 10.410 | 1.00 | 33.08 | C |
| ATOM | 2394 | CE2 | PHE | A | 407 | 14.268 | 39.661 | 11.740 | 1.00 | 33.17 | C |
| ATOM | 2395 | CD2 | PHE | A | 407 | 13.313 | 39.586 | 12.738 | 1.00 | 33.02 | C |
| ATOM | 2396 | C | PHE | A | 407 | 10.230 | 37.786 | 15.374 | 1.00 | 31.70 | C |
| ATOM | 2397 | O | PHE | A | 407 | 11.121 | 37.589 | 16.179 | 1.00 | 31.73 | O |
| ATOM | 2407 | N | LEU | A | 408 | 8.958 | 37.899 | 15.737 | 1.00 | 31.69 | N |
| ATOM | 2408 | CA | LEU | A | 408 | 8.538 | 37.689 | 17.125 | 1.00 | 31.88 | C |
| ATOM | 2409 | CB | LEU | A | 408 | 7.093 | 38.171 | 17.341 | 1.00 | 31.91 | C |
| ATOM | 2410 | CG | LEU | A | 408 | 6.910 | 39.695 | 17.229 | 1.00 | 32.13 | C |
| ATOM | 2411 | CD1 | LEU | A | 408 | 5.445 | 40.098 | 17.261 | 1.00 | 32.21 | C |
| ATOM | 2412 | CD2 | LEU | A | 408 | 7.671 | 40.405 | 18.331 | 1.00 | 32.37 | C |
| ATOM | 2413 | C | LEU | A | 408 | 8.683 | 36.228 | 17.541 | 1.00 | 31.80 | C |
| ATOM | 2414 | O | LEU | A | 408 | 9.014 | 35.936 | 18.695 | 1.00 | 31.72 | O |
| ATOM | 2426 | N | VAL | A | 409 | 8.453 | 35.325 | 16.587 | 1.00 | 32.01 | N |
| ATOM | 2427 | CA | VAL | A | 409 | 8.598 | 33.881 | 16.787 | 1.00 | 31.99 | C |
| ATOM | 2428 | CB | VAL | A | 409 | 7.902 | 33.078 | 15.644 | 1.00 | 32.30 | C |
| ATOM | 2429 | CG1 | VAL | A | 409 | 8.321 | 31.601 | 15.646 | 1.00 | 32.11 | C |
| ATOM | 2430 | CG2 | VAL | A | 409 | 6.376 | 33.196 | 15.742 | 1.00 | 32.29 | C |
| ATOM | 2431 | C | VAL | A | 409 | 10.076 | 33.500 | 16.865 | 1.00 | 31.90 | C |
| ATOM | 2432 | O | VAL | A | 409 | 10.470 | 32.725 | 17.734 | 1.00 | 31.62 | O |
| ATOM | 2442 | N | LEU | A | 410 | 10.885 | 34.056 | 15.962 | 1.00 | 32.04 | N |
| ATOM | 2443 | CA | LEU | A | 410 | 12.315 | 33.753 | 15.901 | 1.00 | 32.18 | C |
| ATOM | 2444 | CB | LEU | A | 410 | 12.926 | 34.321 | 14.620 | 1.00 | 32.17 | C |
| ATOM | 2445 | CG | LEU | A | 410 | 14.415 | 34.035 | 14.370 | 1.00 | 32.90 | C |
| ATOM | 2446 | CD1 | LEU | A | 410 | 14.724 | 32.548 | 14.413 | 1.00 | 32.78 | C |
| ATOM | 2447 | CD2 | LEU | A | 410 | 14.867 | 34.617 | 13.036 | 1.00 | 32.88 | C |
| ATOM | 2448 | C | LEU | A | 410 | 13.057 | 34.289 | 17.132 | 1.00 | 32.17 | C |
| ATOM | 2449 | O | LEU | A | 410 | 13.786 | 33.553 | 17.804 | 1.00 | 31.58 | O |
| ATOM | 2461 | N | PHE | A | 411 | 12.839 | 35.566 | 17.428 | 1.00 | 32.38 | N |
| ATOM | 2462 | CA | PHE | A | 411 | 13.461 | 36.229 | 18.570 | 1.00 | 32.72 | C |
| ATOM | 2463 | CB | PHE | A | 411 | 13.688 | 37.711 | 18.249 | 1.00 | 32.66 | C |
| ATOM | 2464 | CG | PHE | A | 411 | 14.784 | 37.942 | 17.251 | 1.00 | 32.33 | C |
| ATOM | 2465 | CD1 | PHE | A | 411 | 14.503 | 38.077 | 15.901 | 1.00 | 31.89 | C |
| ATOM | 2466 | CE1 | PHE | A | 411 | 15.526 | 38.273 | 14.981 | 1.00 | 31.51 | C |
| ATOM | 2467 | CZ | PHE | A | 411 | 16.829 | 38.331 | 15.409 | 1.00 | 32.07 | C |
| ATOM | 2468 | CE2 | PHE | A | 411 | 17.124 | 38.195 | 16.754 | 1.00 | 32.29 | C |
| ATOM | 2469 | CD2 | PHE | A | 411 | 16.107 | 37.999 | 17.667 | 1.00 | 32.15 | C |
| ATOM | 2470 | C | PHE | A | 411 | 12.637 | 36.053 | 19.854 | 1.00 | 33.12 | C |
| ATOM | 2471 | O | PHE | A | 411 | 12.125 | 37.013 | 20.421 | 1.00 | 33.24 | O |
| ATOM | 2481 | N | SER | A | 412 | 12.544 | 34.808 | 20.311 | 1.00 | 33.72 | N |
| ATOM | 2482 | CA | SER | A | 412 | 11.729 | 34.437 | 21.458 | 1.00 | 34.09 | C |
| ATOM | 2483 | CB | SER | A | 412 | 11.416 | 32.952 | 21.395 | 1.00 | 34.02 | C |
| ATOM | 2484 | OG | SER | A | 412 | 10.653 | 32.555 | 22.515 | 1.00 | 34.93 | O |
| ATOM | 2485 | C | SER | A | 412 | 12.418 | 34.735 | 22.787 | 1.00 | 34.64 | C |
| ATOM | 2486 | O | SER | A | 412 | 13.653 | 34.759 | 22.868 | 1.00 | 34.91 | O |
| ATOM | 2492 | N | LEU | A | 413 | 11.608 | 34.941 | 23.827 | 1.00 | 34.87 | N |
| ATOM | 2493 | CA | LEU | A | 413 | 12.110 | 35.201 | 25.175 | 1.00 | 35.09 | C |
| ATOM | 2494 | CB | LEU | A | 413 | 11.293 | 36.312 | 25.826 | 1.00 | 35.20 | C |
| ATOM | 2495 | CG | LEU | A | 413 | 11.200 | 37.628 | 25.053 | 1.00 | 35.50 | C |
| ATOM | 2496 | CD1 | LEU | A | 413 | 10.327 | 38.626 | 25.815 | 1.00 | 35.90 | C |
| ATOM | 2497 | CD2 | LEU | A | 413 | 12.579 | 38.210 | 24.801 | 1.00 | 35.47 | C |
| ATOM | 2498 | C | LEU | A | 413 | 12.088 | 33.969 | 26.082 | 1.00 | 35.43 | C |
| ATOM | 2499 | O | LEU | A | 413 | 12.534 | 34.048 | 27.238 | 1.00 | 35.80 | O |
| ATOM | 2511 | N | ASP | A | 414 | 11.589 | 32.844 | 25.562 | 1.00 | 35.65 | N |
| ATOM | 2512 | CA | ASP | A | 414 | 11.506 | 31.585 | 26.311 | 1.00 | 35.96 | C |
| ATOM | 2513 | CB | ASP | A | 414 | 10.286 | 30.763 | 25.874 | 1.00 | 36.27 | C |
| ATOM | 2514 | CG | ASP | A | 414 | 8.993 | 31.553 | 25.905 | 1.00 | 37.59 | C |
| ATOM | 2515 | OD1 | ASP | A | 414 | 8.681 | 32.174 | 26.959 | 1.00 | 37.94 | O |
| ATOM | 2516 | OD2 | ASP | A | 414 | 8.225 | 31.587 | 24.910 | 1.00 | 38.86 | O |
| ATOM | 2517 | C | ASP | A | 414 | 12.741 | 30.707 | 26.123 | 1.00 | 35.95 | C |
| ATOM | 2518 | O | ASP | A | 414 | 12.710 | 29.512 | 26.447 | 1.00 | 35.92 | O |
| ATOM | 2523 | N | VAL | A | 415 | 13.818 | 31.288 | 25.599 | 1.00 | 35.97 | N |
| ATOM | 2524 | CA | VAL | A | 415 | 15.025 | 30.537 | 25.274 | 1.00 | 35.90 | C |
| ATOM | 2525 | CB | VAL | A | 415 | 15.536 | 30.881 | 23.864 | 1.00 | 35.88 | C |
| ATOM | 2526 | CG1 | VAL | A | 415 | 16.747 | 30.013 | 23.495 | 1.00 | 35.65 | C |
| ATOM | 2527 | CG2 | VAL | A | 415 | 14.412 | 30.721 | 22.852 | 1.00 | 35.53 | C |
| ATOM | 2528 | C | VAL | A | 415 | 16.112 | 30.827 | 26.300 | 1.00 | 35.98 | C |
| ATOM | 2529 | O | VAL | A | 415 | 16.359 | 31.982 | 26.643 | 1.00 | 35.96 | O |
| ATOM | 2539 | N | LYS | A | 416 | 16.757 | 29.759 | 26.765 | 1.00 | 36.07 | N |
| ATOM | 2540 | CA | LYS | A | 416 | 17.761 | 29.829 | 27.819 | 1.00 | 36.23 | C |
| ATOM | 2541 | CB | LYS | A | 416 | 17.573 | 28.653 | 28.789 | 1.00 | 36.38 | C |
| ATOM | 2542 | CG | LYS | A | 416 | 16.109 | 28.376 | 29.202 | 1.00 | 36.74 | C |
| ATOM | 2543 | CD | LYS | A | 416 | 15.509 | 29.510 | 30.059 | 1.00 | 37.09 | C |
| ATOM | 2544 | CE | LYS | A | 416 | 13.967 | 29.456 | 30.112 | 1.00 | 37.37 | C |
| ATOM | 2545 | NZ | LYS | A | 416 | 13.299 | 30.727 | 29.664 | 1.00 | 37.02 | N |
| ATOM | 2546 | C | LYS | A | 416 | 19.175 | 29.804 | 27.236 | 1.00 | 36.22 | C |
| ATOM | 2547 | O | LYS | A | 416 | 19.365 | 29.562 | 26.044 | 1.00 | 36.79 | O |
| ATOM | 2561 | N | ASN | A | 417 | 20.160 | 30.064 | 28.086 | 1.00 | 36.05 | N |
| ATOM | 2562 | CA | ASN | A | 417 | 21.576 | 29.989 | 27.721 | 1.00 | 36.01 | C |
| ATOM | 2563 | CB | ASN | A | 417 | 21.967 | 28.543 | 27.373 | 1.00 | 36.06 | C |
| ATOM | 2564 | CG | ASN | A | 417 | 21.402 | 27.524 | 28.348 | 1.00 | 36.37 | C |
| ATOM | 2565 | OD1 | ASN | A | 417 | 21.470 | 27.703 | 29.569 | 1.00 | 36.21 | O |
| ATOM | 2566 | ND2 | ASN | A | 417 | 20.849 | 26.437 | 27.810 | 1.00 | 36.12 | N |
| ATOM | 2567 | C | ASN | A | 417 | 22.040 | 30.920 | 26.584 | 1.00 | 35.90 | C |
| ATOM | 2568 | O | ASN | A | 417 | 23.056 | 30.640 | 25.943 | 1.00 | 36.16 | O |
| ATOM | 2575 | N | LEU | A | 418 | 21.327 | 32.019 | 26.335 | 1.00 | 35.55 | N |
| ATOM | 2576 | CA | LEU | A | 418 | 21.749 | 32.971 | 25.301 | 1.00 | 35.25 | C |
| ATOM | 2577 | CB | LEU | A | 418 | 20.573 | 33.817 | 24.807 | 1.00 | 35.25 | C |
| ATOM | 2578 | CG | LEU | A | 418 | 19.549 | 33.127 | 23.903 | 1.00 | 34.74 | C |
| ATOM | 2579 | CD1 | LEU | A | 418 | 18.248 | 33.896 | 23.901 | 1.00 | 34.67 | C |
| ATOM | 2580 | CD2 | LEU | A | 418 | 20.070 | 32.974 | 22.480 | 1.00 | 34.84 | C |
| ATOM | 2581 | C | LEU | A | 418 | 22.858 | 33.891 | 25.811 | 1.00 | 35.30 | C |
| ATOM | 2582 | O | LEU | A | 418 | 22.683 | 34.595 | 26.812 | 1.00 | 35.56 | O |
| ATOM | 2594 | N | GLU | A | 419 | 23.995 | 33.877 | 25.117 | 1.00 | 35.12 | N |
| ATOM | 2595 | CA | GLU | A | 419 | 25.113 | 34.786 | 25.389 | 1.00 | 34.93 | C |
| ATOM | 2596 | CB | GLU | A | 419 | 26.271 | 34.496 | 24.417 | 1.00 | 35.11 | C |
| ATOM | 2597 | CG | GLU | A | 419 | 27.618 | 35.080 | 24.849 | 1.00 | 35.95 | C |
| ATOM | 2598 | CD | GLU | A | 419 | 28.327 | 35.893 | 23.769 | 1.00 | 36.93 | C |
| ATOM | 2599 | OE1 | GLU | A | 419 | 27.903 | 35.855 | 22.591 | 1.00 | 37.21 | O |
| ATOM | 2600 | OE2 | GLU | A | 419 | 29.330 | 36.571 | 24.108 | 1.00 | 37.40 | O |
| ATOM | 2601 | C | GLU | A | 419 | 24.723 | 36.267 | 25.277 | 1.00 | 34.54 | C |
| ATOM | 2602 | O | GLU | A | 419 | 25.243 | 37.112 | 26.010 | 1.00 | 34.74 | O |
| ATOM | 2609 | N | ASN | A | 420 | 23.819 | 36.568 | 24.349 | 1.00 | 34.13 | N |
| ATOM | 2610 | CA | ASN | A | 420 | 23.409 | 37.935 | 24.054 | 1.00 | 33.83 | C |
| ATOM | 2611 | CB | ASN | A | 420 | 23.932 | 38.334 | 22.669 | 1.00 | 33.73 | C |
| ATOM | 2612 | CG | ASN | A | 420 | 23.800 | 39.818 | 22.390 | 1.00 | 33.79 | C |
| ATOM | 2613 | OD1 | ASN | A | 420 | 23.452 | 40.599 | 23.271 | 1.00 | 34.93 | O |
| ATOM | 2614 | ND2 | ASN | A | 420 | 24.084 | 40.215 | 21.154 | 1.00 | 33.36 | N |
| ATOM | 2615 | C | ASN | A | 420 | 21.883 | 38.071 | 24.135 | 1.00 | 33.79 | C |
| ATOM | 2616 | O | ASN | A | 420 | 21.220 | 38.424 | 23.159 | 1.00 | 33.75 | O |
| ATOM | 2623 | N | PHE | A | 421 | 21.336 | 37.796 | 25.317 | 1.00 | 33.81 | N |
| ATOM | 2624 | CA | PHE | A | 421 | 19.901 | 37.932 | 25.567 | 1.00 | 33.85 | C |
| ATOM | 2625 | CB | PHE | A | 421 | 19.539 | 37.391 | 26.959 | 1.00 | 33.86 | C |
| ATOM | 2626 | CG | PHE | A | 421 | 18.063 | 37.121 | 27.148 | 1.00 | 34.21 | C |
| ATOM | 2627 | CD1 | PHE | A | 421 | 17.443 | 36.091 | 26.466 | 1.00 | 34.19 | C |
| ATOM | 2628 | CE1 | PHE | A | 421 | 16.085 | 35.835 | 26.633 | 1.00 | 34.60 | C |
| ATOM | 2629 | CZ | PHE | A | 421 | 15.331 | 36.614 | 27.493 | 1.00 | 34.30 | C |
| ATOM | 2630 | CE2 | PHE | A | 421 | 15.931 | 37.650 | 28.180 | 1.00 | 34.46 | C |
| ATOM | 2631 | CD2 | PHE | A | 421 | 17.296 | 37.899 | 28.008 | 1.00 | 34.89 | C |
| ATOM | 2632 | C | PHE | A | 421 | 19.416 | 39.378 | 25.426 | 1.00 | 33.97 | C |
| ATOM | 2633 | O | PHE | A | 421 | 18.223 | 39.613 | 25.266 | 1.00 | 33.98 | O |
| ATOM | 2643 | N | GLN | A | 422 | 20.335 | 40.338 | 25.482 | 1.00 | 34.18 | N |
| ATOM | 2644 | CA | GLN | A | 422 | 19.990 | 41.756 | 25.366 | 1.00 | 34.43 | C |
| ATOM | 2645 | CB | GLN | A | 422 | 21.178 | 42.632 | 25.786 | 1.00 | 34.51 | C |
| ATOM | 2646 | CG | GLN | A | 422 | 20.986 | 44.111 | 25.486 | 1.00 | 34.92 | C |
| ATOM | 2647 | CD | GLN | A | 422 | 21.739 | 44.997 | 26.435 | 1.00 | 35.70 | C |
| ATOM | 2648 | OE1 | GLN | A | 422 | 21.151 | 45.572 | 27.353 | 1.00 | 36.52 | O |
| ATOM | 2649 | NE2 | GLN | A | 422 | 23.043 | 45.122 | 26.219 | 1.00 | 36.42 | N |
| ATOM | 2650 | C | GLN | A | 422 | 19.532 | 42.147 | 23.958 | 1.00 | 34.40 | C |
| ATOM | 2651 | O | GLN | A | 422 | 18.623 | 42.959 | 23.802 | 1.00 | 34.48 | O |
| ATOM | 2660 | N | LEU | A | 423 | 20.179 | 41.590 | 22.941 | 1.00 | 34.51 | N |
| ATOM | 2661 | CA | LEU | A | 423 | 19.839 | 41.894 | 21.551 | 1.00 | 34.47 | C |
| ATOM | 2662 | CB | LEU | A | 423 | 20.949 | 41.424 | 20.612 | 1.00 | 34.52 | C |
| ATOM | 2663 | CG | LEU | A | 423 | 20.714 | 41.703 | 19.127 | 1.00 | 34.75 | C |
| ATOM | 2664 | CD1 | LEU | A | 423 | 20.524 | 43.205 | 18.886 | 1.00 | 35.02 | C |
| ATOM | 2665 | CD2 | LEU | A | 423 | 21.868 | 41.158 | 18.309 | 1.00 | 34.96 | C |
| ATOM | 2666 | C | LEU | A | 423 | 18.525 | 41.239 | 21.145 | 1.00 | 34.38 | C |
| ATOM | 2667 | O | LEU | A | 423 | 17.789 | 41.783 | 20.318 | 1.00 | 34.30 | O |
| ATOM | 2679 | N | VAL | A | 424 | 18.253 | 40.067 | 21.721 | 1.00 | 34.23 | N |
| ATOM | 2680 | CA | VAL | A | 424 | 17.020 | 39.328 | 21.452 | 1.00 | 34.12 | C |
| ATOM | 2681 | CB | VAL | A | 424 | 17.076 | 37.888 | 22.042 | 1.00 | 34.10 | C |
| ATOM | 2682 | CG1 | VAL | A | 424 | 15.756 | 37.140 | 21.820 | 1.00 | 33.96 | C |
| ATOM | 2683 | CG2 | VAL | A | 424 | 18.234 | 37.109 | 21.430 | 1.00 | 34.07 | C |
| ATOM | 2684 | C | VAL | A | 424 | 15.828 | 40.089 | 22.022 | 1.00 | 34.00 | C |
| ATOM | 2685 | O | VAL | A | 424 | 14.845 | 40.320 | 21.322 | 1.00 | 34.05 | O |
| ATOM | 2695 | N | GLU | A | 425 | 15.937 | 40.485 | 23.289 | 1.00 | 34.07 | N |
| ATOM | 2696 | CA | GLU | A | 425 | 14.914 | 41.288 | 23.974 | 1.00 | 33.99 | C |
| ATOM | 2697 | CB | GLU | A | 425 | 15.299 | 41.510 | 25.455 | 1.00 | 34.16 | C |
| ATOM | 2698 | CG | GLU | A | 425 | 15.027 | 42.898 | 26.059 | 1.00 | 35.44 | C |
| ATOM | 2699 | CD | GLU | A | 425 | 13.681 | 43.018 | 26.762 | 1.00 | 36.48 | C |
| ATOM | 2700 | OE1 | GLU | A | 425 | 13.074 | 41.980 | 27.081 | 1.00 | 38.00 | O |
| ATOM | 2701 | OE2 | GLU | A | 425 | 13.228 | 44.157 | 27.007 | 1.00 | 37.46 | O |
| ATOM | 2702 | C | GLU | A | 425 | 14.669 | 42.610 | 23.241 | 1.00 | 33.64 | C |
| ATOM | 2703 | O | GLU | A | 425 | 13.541 | 43.095 | 23.214 | 1.00 | 33.65 | O |
| ATOM | 2710 | N | GLY | A | 426 | 15.716 | 43.162 | 22.627 | 1.00 | 33.17 | N |
| ATOM | 2711 | CA | GLY | A | 426 | 15.635 | 44.427 | 21.917 | 1.00 | 32.98 | C |
| ATOM | 2712 | C | GLY | A | 426 | 14.831 | 44.395 | 20.633 | 1.00 | 32.88 | C |
| ATOM | 2713 | O | GLY | A | 426 | 14.185 | 45.384 | 20.289 | 1.00 | 32.79 | O |
| ATOM | 2717 | N | VAL | A | 427 | 14.878 | 43.265 | 19.927 | 1.00 | 33.00 | N |
| ATOM | 2718 | CA | VAL | A | 427 | 14.163 | 43.087 | 18.657 | 1.00 | 32.84 | C |
| ATOM | 2719 | CB | VAL | A | 427 | 14.865 | 42.043 | 17.761 | 1.00 | 32.89 | C |
| ATOM | 2720 | CG1 | VAL | A | 427 | 14.070 | 41.782 | 16.473 | 1.00 | 32.87 | C |
| ATOM | 2721 | CG2 | VAL | A | 427 | 16.282 | 42.499 | 17.436 | 1.00 | 32.51 | C |
| ATOM | 2722 | C | VAL | A | 427 | 12.701 | 42.695 | 18.908 | 1.00 | 32.81 | C |
| ATOM | 2723 | O | VAL | A | 427 | 11.814 | 43.125 | 18.188 | 1.00 | 32.41 | O |
| ATOM | 2733 | N | GLN | A | 428 | 12.460 | 41.868 | 19.920 | 1.00 | 32.93 | N |
| ATOM | 2734 | CA | GLN | A | 428 | 11.119 | 41.710 | 20.471 | 1.00 | 33.10 | C |
| ATOM | 2735 | CB | GLN | A | 428 | 11.176 | 40.994 | 21.822 | 1.00 | 33.28 | C |
| ATOM | 2736 | CG | GLN | A | 428 | 10.754 | 39.555 | 21.801 | 1.00 | 34.10 | C |
| ATOM | 2737 | CD | GLN | A | 428 | 9.282 | 39.366 | 21.552 | 1.00 | 34.65 | C |
| ATOM | 2738 | OE1 | GLN | A | 428 | 8.460 | 40.243 | 22.117 | 1.00 | 35.25 | O |
| ATOM | 2739 | NE2 | GLN | A | 428 | 8.891 | 38.432 | 20.850 | 1.00 | 35.09 | N |
| ATOM | 2740 | C | GLN | A | 428 | 10.482 | 43.087 | 20.681 | 1.00 | 33.35 | C |
| ATOM | 2741 | O | GLN | A | 428 | 9.411 | 43.364 | 20.142 | 1.00 | 33.42 | O |
| ATOM | 2750 | N | GLU | A | 429 | 11.162 | 43.934 | 21.466 | 1.00 | 33.23 | N |
| ATOM | 2751 | CA | GLU | A | 429 | 10.696 | 45.283 | 21.812 | 1.00 | 33.25 | C |
| ATOM | 2752 | CB | GLU | A | 429 | 11.751 | 46.022 | 22.677 | 1.00 | 33.43 | C |
| ATOM | 2753 | CG | GLU | A | 429 | 11.819 | 47.550 | 22.523 | 1.00 | 34.14 | C |
| ATOM | 2754 | CD | GLU | A | 429 | 13.055 | 48.191 | 23.168 | 1.00 | 35.07 | C |
| ATOM | 2755 | OE1 | GLU | A | 429 | 14.188 | 47.703 | 22.953 | 1.00 | 35.79 | O |
| ATOM | 2756 | OE2 | GLU | A | 429 | 12.903 | 49.210 | 23.878 | 1.00 | 34.73 | O |
| ATOM | 2757 | C | GLU | A | 429 | 10.372 | 46.093 | 20.562 | 1.00 | 33.07 | C |
| ATOM | 2758 | O | GLU | A | 429 | 9.318 | 46.718 | 20.479 | 1.00 | 33.01 | O |
| ATOM | 2765 | N | GLN | A | 430 | 11.276 | 46.070 | 19.587 | 1.00 | 33.00 | N |
| ATOM | 2766 | CA | GLN | A | 430 | 11.168 | 46.961 | 18.435 | 1.00 | 33.08 | C |
| ATOM | 2767 | CB | GLN | A | 430 | 12.547 | 47.232 | 17.812 | 1.00 | 33.26 | C |
| ATOM | 2768 | CG | GLN | A | 430 | 12.744 | 48.696 | 17.403 | 1.00 | 33.83 | C |
| ATOM | 2769 | CD | GLN | A | 430 | 14.099 | 48.970 | 16.759 | 1.00 | 34.89 | C |
| ATOM | 2770 | OE1 | GLN | A | 430 | 15.018 | 48.152 | 16.845 | 1.00 | 35.54 | O |
| ATOM | 2771 | NE2 | GLN | A | 430 | 14.222 | 50.128 | 16.116 | 1.00 | 35.48 | N |
| ATOM | 2772 | C | GLN | A | 430 | 10.179 | 46.465 | 17.382 | 1.00 | 32.75 | C |
| ATOM | 2773 | O | GLN | A | 430 | 9.515 | 47.270 | 16.742 | 1.00 | 32.67 | O |
| ATOM | 2782 | N | VAL | A | 431 | 10.079 | 45.147 | 17.219 | 1.00 | 32.70 | N |
| ATOM | 2783 | CA | VAL | A | 431 | 9.166 | 44.534 | 16.248 | 1.00 | 32.47 | C |
| ATOM | 2784 | CB | VAL | A | 431 | 9.565 | 43.053 | 15.938 | 1.00 | 32.50 | C |
| ATOM | 2785 | CG1 | VAL | A | 431 | 8.535 | 42.364 | 15.053 | 1.00 | 32.38 | C |
| ATOM | 2786 | CG2 | VAL | A | 431 | 10.923 | 43.000 | 15.259 | 1.00 | 32.68 | C |
| ATOM | 2787 | C | VAL | A | 431 | 7.720 | 44.631 | 16.745 | 1.00 | 32.27 | C |
| ATOM | 2788 | O | VAL | A | 431 | 6.811 | 44.909 | 15.967 | 1.00 | 31.86 | O |
| ATOM | 2798 | N | ASN | A | 432 | 7.525 | 44.406 | 18.042 | 1.00 | 32.24 | N |
| ATOM | 2799 | CA | ASN | A | 432 | 6.228 | 44.590 | 18.696 | 1.00 | 32.24 | C |
| ATOM | 2800 | CB | ASN | A | 432 | 6.338 | 44.257 | 20.186 | 1.00 | 32.22 | C |
| ATOM | 2801 | CG | ASN | A | 432 | 5.068 | 44.576 | 20.965 | 1.00 | 32.31 | C |
| ATOM | 2802 | OD1 | ASN | A | 432 | 4.267 | 43.697 | 21.247 | 1.00 | 32.62 | O |
| ATOM | 2803 | ND2 | ASN | A | 432 | 4.898 | 45.837 | 21.340 | 1.00 | 33.78 | N |
| ATOM | 2804 | C | ASN | A | 432 | 5.731 | 46.014 | 18.538 | 1.00 | 32.55 | C |
| ATOM | 2805 | O | ASN | A | 432 | 4.562 | 46.233 | 18.248 | 1.00 | 33.02 | O |
| ATOM | 2812 | N | ALA | A | 433 | 6.631 | 46.974 | 18.751 | 1.00 | 32.58 | N |
| ATOM | 2813 | CA | ALA | A | 433 | 6.318 | 48.394 | 18.658 | 1.00 | 32.33 | C |
| ATOM | 2814 | CB | ALA | A | 433 | 7.425 | 49.208 | 19.301 | 1.00 | 32.37 | C |
| ATOM | 2815 | C | ALA | A | 433 | 6.118 | 48.842 | 17.212 | 1.00 | 32.14 | C |
| ATOM | 2816 | O | ALA | A | 433 | 5.382 | 49.789 | 16.954 | 1.00 | 32.32 | O |
| ATOM | 2822 | N | ALA | A | 434 | 6.785 | 48.170 | 16.280 | 1.00 | 31.90 | N |
| ATOM | 2823 | CA | ALA | A | 434 | 6.652 | 48.460 | 14.858 | 1.00 | 31.78 | C |
| ATOM | 2824 | CB | ALA | A | 434 | 7.793 | 47.832 | 14.087 | 1.00 | 31.66 | C |
| ATOM | 2825 | C | ALA | A | 434 | 5.320 | 47.945 | 14.336 | 1.00 | 31.96 | C |
| ATOM | 2826 | O | ALA | A | 434 | 4.703 | 48.579 | 13.492 | 1.00 | 31.92 | O |
| ATOM | 2832 | N | LEU | A | 435 | 4.888 | 46.792 | 14.848 | 1.00 | 32.20 | N |
| ATOM | 2833 | CA | LEU | A | 435 | 3.601 | 46.192 | 14.498 | 1.00 | 32.25 | C |
| ATOM | 2834 | CB | LEU | A | 435 | 3.544 | 44.731 | 14.974 | 1.00 | 32.26 | C |
| ATOM | 2835 | CG | LEU | A | 435 | 2.272 | 43.954 | 14.618 | 1.00 | 32.31 | C |
| ATOM | 2836 | CD1 | LEU | A | 435 | 2.268 | 43.556 | 13.156 | 1.00 | 32.62 | C |
| ATOM | 2837 | CD2 | LEU | A | 435 | 2.124 | 42.733 | 15.491 | 1.00 | 33.04 | C |
| ATOM | 2838 | C | LEU | A | 435 | 2.458 | 46.977 | 15.128 | 1.00 | 32.23 | C |
| ATOM | 2839 | O | LEU | A | 435 | 1.407 | 47.161 | 14.526 | 1.00 | 32.37 | O |
| ATOM | 2851 | N | LEU | A | 436 | 2.675 | 47.422 | 16.357 | 1.00 | 32.24 | N |
| ATOM | 2852 | CA | LEU | A | 436 | 1.726 | 48.261 | 17.073 | 1.00 | 32.35 | C |
| ATOM | 2853 | CB | LEU | A | 436 | 2.281 | 48.547 | 18.471 | 1.00 | 32.26 | C |
| ATOM | 2854 | CG | LEU | A | 436 | 1.439 | 49.268 | 19.516 | 1.00 | 31.74 | C |
| ATOM | 2855 | CD1 | LEU | A | 436 | 0.046 | 48.677 | 19.633 | 1.00 | 31.49 | C |
| ATOM | 2856 | CD2 | LEU | A | 436 | 2.170 | 49.207 | 20.850 | 1.00 | 30.94 | C |
| ATOM | 2857 | C | LEU | A | 436 | 1.497 | 49.560 | 16.303 | 1.00 | 32.58 | C |
| ATOM | 2858 | O | LEU | A | 436 | 0.406 | 50.109 | 16.319 | 1.00 | 32.74 | O |
| ATOM | 2870 | N | ASP | A | 437 | 2.540 | 50.009 | 15.611 | 1.00 | 32.93 | N |
| ATOM | 2871 | CA | ASP | A | 437 | 2.545 | 51.231 | 14.809 | 1.00 | 33.34 | C |
| ATOM | 2872 | CB | ASP | A | 437 | 4.004 | 51.615 | 14.529 | 1.00 | 33.56 | C |
| ATOM | 2873 | CG | ASP | A | 437 | 4.180 | 53.067 | 14.187 | 1.00 | 34.67 | C |
| ATOM | 2874 | OD1 | ASP | A | 437 | 3.873 | 53.464 | 13.039 | 1.00 | 36.40 | O |
| ATOM | 2875 | OD2 | ASP | A | 437 | 4.654 | 53.887 | 15.000 | 1.00 | 36.60 | O |
| ATOM | 2876 | C | ASP | A | 437 | 1.809 | 51.053 | 13.478 | 1.00 | 33.26 | C |
| ATOM | 2877 | O | ASP | A | 437 | 1.043 | 51.921 | 13.065 | 1.00 | 33.14 | O |
| ATOM | 2882 | N | TYR | A | 438 | 2.058 | 49.924 | 12.816 | 1.00 | 33.33 | N |
| ATOM | 2883 | CA | TYR | A | 438 | 1.507 | 49.629 | 11.492 | 1.00 | 33.23 | C |
| ATOM | 2884 | CB | TYR | A | 438 | 2.209 | 48.402 | 10.892 | 1.00 | 33.23 | C |
| ATOM | 2885 | CG | TYR | A | 438 | 1.564 | 47.842 | 9.638 | 1.00 | 33.26 | C |
| ATOM | 2886 | CD1 | TYR | A | 438 | 1.951 | 48.275 | 8.367 | 1.00 | 32.96 | C |
| ATOM | 2887 | CE1 | TYR | A | 438 | 1.358 | 47.758 | 7.219 | 1.00 | 32.44 | C |
| ATOM | 2888 | CZ | TYR | A | 438 | 0.374 | 46.791 | 7.339 | 1.00 | 33.21 | C |
| ATOM | 2889 | OH | TYR | A | 438 | −0.226 | 46.253 | 6.224 | 1.00 | 33.09 | O |
| ATOM | 2890 | CE2 | TYR | A | 438 | −0.018 | 46.346 | 8.584 | 1.00 | 33.50 | C |
| ATOM | 2891 | CD2 | TYR | A | 438 | 0.578 | 46.867 | 9.722 | 1.00 | 33.58 | C |
| ATOM | 2892 | C | TYR | A | 438 | 0.001 | 49.391 | 11.538 | 1.00 | 33.26 | C |
| ATOM | 2893 | O | TYR | A | 438 | −0.710 | 49.742 | 10.603 | 1.00 | 33.45 | O |
| ATOM | 2903 | N | THR | A | 439 | −0.478 | 48.787 | 12.623 | 1.00 | 33.17 | N |
| ATOM | 2904 | CA | THR | A | 439 | −1.904 | 48.505 | 12.784 | 1.00 | 32.96 | C |
| ATOM | 2905 | CB | THR | A | 439 | −2.149 | 47.460 | 13.891 | 1.00 | 32.82 | C |
| ATOM | 2906 | OG1 | THR | A | 439 | −1.494 | 47.864 | 15.099 | 1.00 | 32.55 | O |
| ATOM | 2907 | CG2 | THR | A | 439 | −1.515 | 46.123 | 13.543 | 1.00 | 32.81 | C |
| ATOM | 2908 | C | THR | A | 439 | −2.645 | 49.779 | 13.135 | 1.00 | 32.93 | C |
| ATOM | 2909 | O | THR | A | 439 | −3.839 | 49.899 | 12.876 | 1.00 | 32.78 | O |
| ATOM | 2917 | N | MET | A | 440 | −1.930 | 50.721 | 13.740 | 1.00 | 33.04 | N |
| ATOM | 2918 | CA | MET | A | 440 | −2.502 | 52.005 | 14.128 | 1.00 | 33.23 | C |
| ATOM | 2919 | CB | MET | A | 440 | −1.609 | 52.650 | 15.192 | 1.00 | 33.26 | C |
| ATOM | 2920 | CG | MET | A | 440 | −2.239 | 53.812 | 15.933 | 1.00 | 33.85 | C |
| ATOM | 2921 | SD | MET | A | 440 | −1.093 | 55.177 | 16.191 | 1.00 | 33.56 | S |
| ATOM | 2922 | CE | MET | A | 440 | −1.901 | 56.509 | 15.268 | 1.00 | 35.26 | C |
| ATOM | 2923 | C | MET | A | 440 | −2.696 | 52.949 | 12.921 | 1.00 | 33.22 | C |
| ATOM | 2924 | O | MET | A | 440 | −3.690 | 53.671 | 12.851 | 1.00 | 33.10 | O |
| ATOM | 2934 | N | CYS | A | 441 | −1.760 | 52.923 | 11.971 | 1.00 | 33.36 | N |
| ATOM | 2935 | CA | CYS | A | 441 | −1.806 | 53.808 | 10.803 | 1.00 | 33.61 | C |
| ATOM | 2936 | CB | CYS | A | 441 | −0.400 | 54.047 | 10.249 | 1.00 | 33.56 | C |
| ATOM | 2937 | SG | CYS | A | 441 | 0.735 | 54.841 | 11.403 | 1.00 | 33.54 | S |
| ATOM | 2938 | C | CYS | A | 441 | −2.676 | 53.252 | 9.682 | 1.00 | 33.85 | C |
| ATOM | 2939 | O | CYS | A | 441 | −3.497 | 53.968 | 9.113 | 1.00 | 34.02 | O |
| ATOM | 2945 | N | ASN | A | 442 | −2.473 | 51.980 | 9.354 | 1.00 | 34.11 | N |
| ATOM | 2946 | CA | ASN | A | 442 | −3.158 | 51.354 | 8.224 | 1.00 | 34.35 | C |
| ATOM | 2947 | CB | ASN | A | 442 | −2.328 | 50.186 | 7.676 | 1.00 | 34.38 | C |
| ATOM | 2948 | CG | ASN | A | 442 | −1.145 | 50.655 | 6.842 | 1.00 | 34.63 | C |
| ATOM | 2949 | OD1 | ASN | A | 442 | −1.280 | 50.902 | 5.640 | 1.00 | 34.76 | O |
| ATOM | 2950 | ND2 | ASN | A | 442 | 0.016 | 50.793 | 7.478 | 1.00 | 34.05 | N |
| ATOM | 2951 | C | ASN | A | 442 | −4.583 | 50.894 | 8.550 | 1.00 | 34.46 | C |
| ATOM | 2952 | O | ASN | A | 442 | −5.473 | 50.998 | 7.708 | 1.00 | 34.54 | O |
| ATOM | 2959 | N | TYR | A | 443 | −4.796 | 50.393 | 9.766 | 1.00 | 34.54 | N |
| ATOM | 2960 | CA | TYR | A | 443 | −6.117 | 49.942 | 10.203 | 1.00 | 34.56 | C |
| ATOM | 2961 | CB | TYR | A | 443 | −6.081 | 48.437 | 10.507 | 1.00 | 34.69 | C |
| ATOM | 2962 | CG | TYR | A | 443 | −5.607 | 47.585 | 9.348 | 1.00 | 35.03 | C |
| ATOM | 2963 | CD1 | TYR | A | 443 | −4.317 | 47.062 | 9.322 | 1.00 | 35.26 | C |
| ATOM | 2964 | CE1 | TYR | A | 443 | −3.879 | 46.284 | 8.257 | 1.00 | 35.43 | C |
| ATOM | 2965 | CZ | TYR | A | 443 | −4.734 | 46.023 | 7.200 | 1.00 | 35.55 | C |
| ATOM | 2966 | OH | TYR | A | 443 | −4.310 | 45.248 | 6.145 | 1.00 | 36.22 | O |
| ATOM | 2967 | CE2 | TYR | A | 443 | −6.017 | 46.529 | 7.203 | 1.00 | 35.32 | C |
| ATOM | 2968 | CD2 | TYR | A | 443 | −6.447 | 47.306 | 8.273 | 1.00 | 35.53 | C |
| ATOM | 2969 | C | TYR | A | 443 | −6.589 | 50.729 | 11.436 | 1.00 | 34.48 | C |
| ATOM | 2970 | O | TYR | A | 443 | −6.751 | 50.153 | 12.512 | 1.00 | 34.47 | O |
| ATOM | 2980 | N | PRO | A | 444 | −6.830 | 52.034 | 11.281 | 1.00 | 34.47 | N |
| ATOM | 2981 | CA | PRO | A | 444 | −7.146 | 52.901 | 12.428 | 1.00 | 34.45 | C |
| ATOM | 2982 | CB | PRO | A | 444 | −7.277 | 54.300 | 11.801 | 1.00 | 34.39 | C |
| ATOM | 2983 | CG | PRO | A | 444 | −7.520 | 54.072 | 10.358 | 1.00 | 34.34 | C |
| ATOM | 2984 | CD | PRO | A | 444 | −6.842 | 52.784 | 10.011 | 1.00 | 34.40 | C |
| ATOM | 2985 | C | PRO | A | 444 | −8.431 | 52.517 | 13.159 | 1.00 | 34.48 | C |
| ATOM | 2986 | O | PRO | A | 444 | −8.387 | 52.316 | 14.369 | 1.00 | 34.64 | O |
| ATOM | 2994 | N | GLN | A | 445 | −9.540 | 52.393 | 12.434 | 1.00 | 34.49 | N |
| ATOM | 2995 | CA | GLN | A | 445 | −10.833 | 52.062 | 13.046 | 1.00 | 34.45 | C |
| ATOM | 2996 | CB | GLN | A | 445 | −12.003 | 52.569 | 12.182 | 1.00 | 34.59 | C |
| ATOM | 2997 | CG | GLN | A | 445 | −12.002 | 52.095 | 10.724 | 1.00 | 34.96 | C |
| ATOM | 2998 | CD | GLN | A | 445 | −11.600 | 53.191 | 9.740 | 1.00 | 35.52 | C |
| ATOM | 2999 | OE1 | GLN | A | 445 | −12.241 | 54.244 | 9.675 | 1.00 | 35.47 | O |
| ATOM | 3000 | NE2 | GLN | A | 445 | −10.540 | 52.943 | 8.973 | 1.00 | 36.05 | N |
| ATOM | 3001 | C | GLN | A | 445 | −10.993 | 50.566 | 13.358 | 1.00 | 34.33 | C |
| ATOM | 3002 | O | GLN | A | 445 | −12.031 | 50.146 | 13.867 | 1.00 | 34.22 | O |
| ATOM | 3011 | N | GLN | A | 446 | −9.972 | 49.767 | 13.053 | 1.00 | 34.25 | N |
| ATOM | 3012 | CA | GLN | A | 446 | −9.913 | 48.378 | 13.503 | 1.00 | 34.22 | C |
| ATOM | 3013 | CB | GLN | A | 446 | −9.561 | 47.441 | 12.342 | 1.00 | 34.33 | C |
| ATOM | 3014 | CG | GLN | A | 446 | −10.776 | 46.944 | 11.544 | 1.00 | 34.64 | C |
| ATOM | 3015 | CD | GLN | A | 446 | −10.784 | 47.387 | 10.084 | 1.00 | 35.25 | C |
| ATOM | 3016 | OE1 | GLN | A | 446 | −9.604 | 47.648 | 9.520 | 1.00 | 35.55 | O |
| ATOM | 3017 | NE2 | GLN | A | 446 | −11.852 | 47.479 | 9.473 | 1.00 | 35.16 | N |
| ATOM | 3018 | C | GLN | A | 446 | −8.893 | 48.270 | 14.640 | 1.00 | 34.05 | C |
| ATOM | 3019 | O | GLN | A | 446 | −7.739 | 47.877 | 14.436 | 1.00 | 34.11 | O |
| ATOM | 3028 | N | THR | A | 447 | −9.349 | 48.628 | 15.838 | 1.00 | 33.83 | N |
| ATOM | 3029 | CA | THR | A | 447 | −8.504 | 48.697 | 17.029 | 1.00 | 33.62 | C |
| ATOM | 3030 | CB | THR | A | 447 | −9.275 | 49.356 | 18.199 | 1.00 | 33.60 | C |
| ATOM | 3031 | OG1 | THR | A | 447 | −9.974 | 50.519 | 17.745 | 1.00 | 33.72 | O |
| ATOM | 3032 | CG2 | THR | A | 447 | −8.320 | 49.893 | 19.245 | 1.00 | 33.65 | C |
| ATOM | 3033 | C | THR | A | 447 | −8.043 | 47.318 | 17.469 | 1.00 | 33.38 | C |
| ATOM | 3034 | O | THR | A | 447 | −6.967 | 47.167 | 18.037 | 1.00 | 33.24 | O |
| ATOM | 3042 | N | GLU | A | 448 | −8.874 | 46.316 | 17.205 | 1.00 | 33.33 | N |
| ATOM | 3043 | CA | GLU | A | 448 | −8.607 | 44.953 | 17.643 | 1.00 | 33.33 | C |
| ATOM | 3044 | CB | GLU | A | 448 | −9.902 | 44.117 | 17.598 | 1.00 | 33.57 | C |
| ATOM | 3045 | CG | GLU | A | 448 | −10.396 | 43.706 | 16.208 | 1.00 | 34.53 | C |
| ATOM | 3046 | CD | GLU | A | 448 | −11.412 | 44.661 | 15.602 | 1.00 | 35.80 | C |
| ATOM | 3047 | OE1 | GLU | A | 448 | −12.340 | 45.099 | 16.315 | 1.00 | 37.27 | O |
| ATOM | 3048 | OE2 | GLU | A | 448 | −11.284 | 44.972 | 14.396 | 1.00 | 36.76 | O |
| ATOM | 3049 | C | GLU | A | 448 | −7.481 | 44.251 | 16.877 | 1.00 | 32.92 | C |
| ATOM | 3050 | O | GLU | A | 448 | −6.917 | 43.304 | 17.390 | 1.00 | 32.96 | O |
| ATOM | 3057 | N | LYS | A | 449 | −7.151 | 44.727 | 15.675 | 1.00 | 32.74 | N |
| ATOM | 3058 | CA | LYS | A | 449 | −6.231 | 44.028 | 14.758 | 1.00 | 32.48 | C |
| ATOM | 3059 | CB | LYS | A | 449 | −5.880 | 44.903 | 13.552 | 1.00 | 32.68 | C |
| ATOM | 3060 | CG | LYS | A | 449 | −6.904 | 44.858 | 12.433 | 1.00 | 33.22 | C |
| ATOM | 3061 | CD | LYS | A | 449 | −6.519 | 43.873 | 11.331 | 1.00 | 33.05 | C |
| ATOM | 3062 | CE | LYS | A | 449 | −7.736 | 43.501 | 10.482 | 1.00 | 33.45 | C |
| ATOM | 3063 | NZ | LYS | A | 449 | −7.588 | 43.893 | 9.055 | 1.00 | 34.02 | N |
| ATOM | 3064 | C | LYS | A | 449 | −4.934 | 43.574 | 15.391 | 1.00 | 32.06 | C |
| ATOM | 3065 | O | LYS | A | 449 | −4.565 | 42.413 | 15.269 | 1.00 | 31.93 | O |
| ATOM | 3079 | N | PHE | A | 450 | −4.236 | 44.502 | 16.034 | 1.00 | 31.57 | N |
| ATOM | 3080 | CA | PHE | A | 450 | −2.958 | 44.213 | 16.679 | 1.00 | 31.15 | C |
| ATOM | 3081 | CB | PHE | A | 450 | −2.484 | 45.446 | 17.457 | 1.00 | 30.72 | C |
| ATOM | 3082 | CG | PHE | A | 450 | −1.300 | 45.200 | 18.338 | 1.00 | 29.50 | C |
| ATOM | 3083 | CD1 | PHE | A | 450 | −0.022 | 45.170 | 17.808 | 1.00 | 28.67 | C |
| ATOM | 3084 | CE1 | PHE | A | 450 | 1.081 | 44.946 | 18.624 | 1.00 | 28.48 | C |
| ATOM | 3085 | CZ | PHE | A | 450 | 0.907 | 44.756 | 19.985 | 1.00 | 28.29 | C |
| ATOM | 3086 | CE2 | PHE | A | 450 | −0.367 | 44.780 | 20.527 | 1.00 | 28.63 | C |
| ATOM | 3087 | CD2 | PHE | A | 450 | −1.463 | 45.003 | 19.705 | 1.00 | 28.93 | C |
| ATOM | 3088 | C | PHE | A | 450 | −3.059 | 42.994 | 17.605 | 1.00 | 31.37 | C |
| ATOM | 3089 | O | PHE | A | 450 | −2.212 | 42.106 | 17.564 | 1.00 | 31.23 | O |
| ATOM | 3099 | N | GLY | A | 451 | −4.097 | 42.971 | 18.435 | 1.00 | 31.62 | N |
| ATOM | 3100 | CA | GLY | A | 451 | −4.291 | 41.923 | 19.420 | 1.00 | 31.69 | C |
| ATOM | 3101 | C | GLY | A | 451 | −4.716 | 40.602 | 18.826 | 1.00 | 31.98 | C |
| ATOM | 3102 | O | GLY | A | 451 | −4.415 | 39.550 | 19.382 | 1.00 | 31.94 | O |
| ATOM | 3106 | N | GLN | A | 452 | −5.422 | 40.648 | 17.701 | 1.00 | 32.21 | N |
| ATOM | 3107 | CA | GLN | A | 452 | −5.818 | 39.431 | 17.000 | 1.00 | 32.35 | C |
| ATOM | 3108 | CB | GLN | A | 452 | −6.875 | 39.735 | 15.937 | 1.00 | 32.52 | C |
| ATOM | 3109 | CG | GLN | A | 452 | −8.138 | 40.366 | 16.506 | 1.00 | 33.79 | C |
| ATOM | 3110 | CD | GLN | A | 452 | −9.334 | 40.285 | 15.570 | 1.00 | 35.43 | C |
| ATOM | 3111 | OE1 | GLN | A | 452 | −10.201 | 39.422 | 15.738 | 1.00 | 38.13 | O |
| ATOM | 3112 | NE2 | GLN | A | 452 | −9.397 | 41.195 | 14.600 | 1.00 | 35.46 | N |
| ATOM | 3113 | C | GLN | A | 452 | −4.607 | 38.737 | 16.378 | 1.00 | 32.26 | C |
| ATOM | 3114 | O | GLN | A | 452 | −4.578 | 37.510 | 16.284 | 1.00 | 32.33 | O |
| ATOM | 3123 | N | LEU | A | 453 | −3.608 | 39.524 | 15.972 | 1.00 | 32.13 | N |
| ATOM | 3124 | CA | LEU | A | 453 | −2.343 | 38.999 | 15.462 | 1.00 | 31.92 | C |
| ATOM | 3125 | CB | LEU | A | 453 | −1.537 | 40.089 | 14.760 | 1.00 | 31.93 | C |
| ATOM | 3126 | CG | LEU | A | 453 | −2.164 | 40.745 | 13.533 | 1.00 | 32.30 | C |
| ATOM | 3127 | CD1 | LEU | A | 453 | −1.474 | 42.079 | 13.226 | 1.00 | 32.28 | C |
| ATOM | 3128 | CD2 | LEU | A | 453 | −2.098 | 39.818 | 12.343 | 1.00 | 32.77 | C |
| ATOM | 3129 | C | LEU | A | 453 | −1.490 | 38.388 | 16.573 | 1.00 | 31.82 | C |
| ATOM | 3130 | O | LEU | A | 453 | −0.947 | 37.308 | 16.394 | 1.00 | 31.92 | O |
| ATOM | 3142 | N | LEU | A | 454 | −1.362 | 39.066 | 17.712 | 1.00 | 31.54 | N |
| ATOM | 3143 | CA | LEU | A | 454 | −0.569 | 38.523 | 18.818 | 1.00 | 31.63 | C |
| ATOM | 3144 | CB | LEU | A | 454 | −0.344 | 39.571 | 19.910 | 1.00 | 31.56 | C |
| ATOM | 3145 | CG | LEU | A | 454 | 0.513 | 40.814 | 19.623 | 1.00 | 32.05 | C |
| ATOM | 3146 | CD1 | LEU | A | 454 | 0.722 | 41.576 | 20.917 | 1.00 | 32.01 | C |
| ATOM | 3147 | CD2 | LEU | A | 454 | 1.845 | 40.498 | 18.974 | 1.00 | 31.97 | C |
| ATOM | 3148 | C | LEU | A | 454 | −1.180 | 37.251 | 19.451 | 1.00 | 31.66 | C |
| ATOM | 3149 | O | LEU | A | 454 | −0.452 | 36.372 | 19.909 | 1.00 | 31.41 | O |
| ATOM | 3161 | N | LEU | A | 455 | −2.504 | 37.150 | 19.481 | 1.00 | 31.67 | N |
| ATOM | 3162 | CA | LEU | A | 455 | −3.161 | 35.984 | 20.086 | 1.00 | 32.10 | C |
| ATOM | 3163 | CB | LEU | A | 455 | −4.616 | 36.296 | 20.454 | 1.00 | 32.04 | C |
| ATOM | 3164 | CG | LEU | A | 455 | −4.697 | 37.248 | 21.650 | 1.00 | 33.04 | C |
| ATOM | 3165 | CD1 | LEU | A | 455 | −6.059 | 37.910 | 21.728 | 1.00 | 33.89 | C |
| ATOM | 3166 | CD2 | LEU | A | 455 | −4.368 | 36.542 | 22.965 | 1.00 | 33.05 | C |
| ATOM | 3167 | C | LEU | A | 455 | −3.083 | 34.746 | 19.186 | 1.00 | 31.85 | C |
| ATOM | 3168 | O | LEU | A | 455 | −3.350 | 33.631 | 19.627 | 1.00 | 31.09 | O |
| ATOM | 3180 | N | ARG | A | 456 | −2.690 | 34.966 | 17.934 | 1.00 | 32.10 | N |
| ATOM | 3181 | CA | ARG | A | 456 | −2.455 | 33.891 | 16.989 | 1.00 | 32.26 | C |
| ATOM | 3182 | CB | ARG | A | 456 | −2.549 | 34.416 | 15.550 | 1.00 | 32.15 | C |
| ATOM | 3183 | CG | ARG | A | 456 | −3.704 | 33.816 | 14.769 | 1.00 | 33.20 | C |
| ATOM | 3184 | CD | ARG | A | 456 | −5.022 | 34.579 | 14.856 | 1.00 | 34.71 | C |
| ATOM | 3185 | NE | ARG | A | 456 | −5.486 | 34.793 | 16.233 | 1.00 | 35.67 | N |
| ATOM | 3186 | CZ | ARG | A | 456 | −6.333 | 34.019 | 16.888 | 1.00 | 35.84 | C |
| ATOM | 3187 | NH1 | ARG | A | 456 | −6.840 | 32.938 | 16.326 | 1.00 | 38.46 | N |
| ATOM | 3188 | NH2 | ARG | A | 456 | −6.673 | 34.316 | 18.130 | 1.00 | 36.28 | N |
| ATOM | 3189 | C | ARG | A | 456 | −1.117 | 33.187 | 17.232 | 1.00 | 32.30 | C |
| ATOM | 3190 | O | ARG | A | 456 | −0.893 | 32.110 | 16.693 | 1.00 | 32.44 | O |
| ATOM | 3204 | N | LEU | A | 457 | −0.250 | 33.780 | 18.052 | 1.00 | 32.32 | N |
| ATOM | 3205 | CA | LEU | A | 457 | 1.077 | 33.218 | 18.329 | 1.00 | 32.42 | C |
| ATOM | 3206 | CB | LEU | A | 457 | 2.030 | 34.283 | 18.903 | 1.00 | 32.46 | C |
| ATOM | 3207 | CG | LEU | A | 457 | 2.618 | 35.236 | 17.866 | 1.00 | 32.71 | C |
| ATOM | 3208 | CD1 | LEU | A | 457 | 3.480 | 36.278 | 18.548 | 1.00 | 32.67 | C |
| ATOM | 3209 | CD2 | LEU | A | 457 | 3.400 | 34.468 | 16.802 | 1.00 | 33.11 | C |
| ATOM | 3210 | C | LEU | A | 457 | 1.081 | 31.978 | 19.222 | 1.00 | 32.32 | C |
| ATOM | 3211 | O | LEU | A | 457 | 1.753 | 31.009 | 18.887 | 1.00 | 31.91 | O |
| ATOM | 3223 | N | PRO | A | 458 | 0.389 | 32.008 | 20.363 | 1.00 | 32.34 | N |
| ATOM | 3224 | CA | PRO | A | 458 | 0.168 | 30.789 | 21.162 | 1.00 | 32.26 | C |
| ATOM | 3225 | CB | PRO | A | 458 | −0.832 | 31.244 | 22.230 | 1.00 | 32.08 | C |
| ATOM | 3226 | CG | PRO | A | 458 | −0.601 | 32.694 | 22.379 | 1.00 | 32.19 | C |
| ATOM | 3227 | CD | PRO | A | 458 | −0.184 | 33.194 | 21.023 | 1.00 | 32.34 | C |
| ATOM | 3228 | C | PRO | A | 458 | −0.412 | 29.612 | 20.372 | 1.00 | 32.23 | C |
| ATOM | 3229 | O | PRO | A | 458 | −0.053 | 28.468 | 20.620 | 1.00 | 32.17 | O |
| ATOM | 3237 | N | GLU | A | 459 | −1.308 | 29.893 | 19.439 | 1.00 | 32.43 | N |
| ATOM | 3238 | CA | GLU | A | 459 | −1.924 | 28.849 | 18.629 | 1.00 | 32.65 | C |
| ATOM | 3239 | CB | GLU | A | 459 | −3.102 | 29.428 | 17.851 | 1.00 | 32.67 | C |
| ATOM | 3240 | CG | GLU | A | 459 | −4.224 | 29.937 | 18.751 | 1.00 | 32.73 | C |
| ATOM | 3241 | CD | GLU | A | 459 | −5.343 | 30.605 | 17.981 | 1.00 | 32.06 | C |
| ATOM | 3242 | OE1 | GLU | A | 459 | −5.110 | 31.021 | 16.827 | 1.00 | 30.91 | O |
| ATOM | 3243 | OE2 | GLU | A | 459 | −6.457 | 30.706 | 18.531 | 1.00 | 32.50 | O |
| ATOM | 3244 | C | GLU | A | 459 | −0.931 | 28.205 | 17.661 | 1.00 | 32.91 | C |
| ATOM | 3245 | O | GLU | A | 459 | −0.988 | 27.000 | 17.422 | 1.00 | 33.42 | O |
| ATOM | 3252 | N | ILE | A | 460 | −0.042 | 29.020 | 17.100 | 1.00 | 32.86 | N |
| ATOM | 3253 | CA | ILE | A | 460 | 0.988 | 28.569 | 16.174 | 1.00 | 32.91 | C |
| ATOM | 3254 | CB | ILE | A | 460 | 1.668 | 29.794 | 15.513 | 1.00 | 32.94 | C |
| ATOM | 3255 | CG1 | ILE | A | 460 | 0.821 | 30.292 | 14.337 | 1.00 | 33.27 | C |
| ATOM | 3256 | CD1 | ILE | A | 460 | 1.113 | 31.729 | 13.930 | 1.00 | 33.07 | C |
| ATOM | 3257 | CG2 | ILE | A | 460 | 3.082 | 29.469 | 15.057 | 1.00 | 33.01 | C |
| ATOM | 3258 | C | ILE | A | 460 | 2.018 | 27.715 | 16.903 | 1.00 | 32.83 | C |
| ATOM | 3259 | O | ILE | A | 460 | 2.510 | 26.723 | 16.372 | 1.00 | 33.26 | O |
| ATOM | 3271 | N | ARG | A | 461 | 2.356 | 28.128 | 18.112 | 1.00 | 32.74 | N |
| ATOM | 3272 | CA | ARG | A | 461 | 3.226 | 27.366 | 18.977 | 1.00 | 32.81 | C |
| ATOM | 3273 | CB | ARG | A | 461 | 3.445 | 28.129 | 20.291 | 1.00 | 32.80 | C |
| ATOM | 3274 | CG | ARG | A | 461 | 4.155 | 27.355 | 21.403 | 1.00 | 33.87 | C |
| ATOM | 3275 | CD | ARG | A | 461 | 5.379 | 26.565 | 20.956 | 1.00 | 35.56 | C |
| ATOM | 3276 | NE | ARG | A | 461 | 6.614 | 27.122 | 21.503 | 1.00 | 37.83 | N |
| ATOM | 3277 | CZ | ARG | A | 461 | 7.793 | 27.169 | 20.873 | 1.00 | 39.34 | C |
| ATOM | 3278 | NH1 | ARG | A | 461 | 7.951 | 26.686 | 19.646 | 1.00 | 39.93 | N |
| ATOM | 3279 | NH2 | ARG | A | 461 | 8.833 | 27.710 | 21.484 | 1.00 | 40.57 | N |
| ATOM | 3280 | C | ARG | A | 461 | 2.626 | 25.974 | 19.232 | 1.00 | 32.85 | C |
| ATOM | 3281 | O | ARG | A | 461 | 3.331 | 24.976 | 19.155 | 1.00 | 32.83 | O |
| ATOM | 3295 | N | ALA | A | 462 | 1.321 | 25.925 | 19.492 | 1.00 | 32.83 | N |
| ATOM | 3296 | CA | ALA | A | 462 | 0.625 | 24.696 | 19.858 | 1.00 | 32.78 | C |
| ATOM | 3297 | CB | ALA | A | 462 | −0.743 | 25.017 | 20.433 | 1.00 | 32.94 | C |
| ATOM | 3298 | C | ALA | A | 462 | 0.475 | 23.746 | 18.684 | 1.00 | 32.82 | C |
| ATOM | 3299 | O | ALA | A | 462 | 0.680 | 22.550 | 18.838 | 1.00 | 32.87 | O |
| ATOM | 3305 | N | ILE | A | 463 | 0.115 | 24.271 | 17.515 | 1.00 | 32.96 | N |
| ATOM | 3306 | CA | ILE | A | 463 | 0.020 | 23.453 | 16.311 | 1.00 | 33.05 | C |
| ATOM | 3307 | CB | ILE | A | 463 | −0.512 | 24.264 | 15.118 | 1.00 | 33.15 | C |
| ATOM | 3308 | CG1 | ILE | A | 463 | −1.999 | 24.554 | 15.295 | 1.00 | 33.66 | C |
| ATOM | 3309 | CD1 | ILE | A | 463 | −2.543 | 25.616 | 14.336 | 1.00 | 33.68 | C |
| ATOM | 3310 | CG2 | ILE | A | 463 | −0.330 | 23.490 | 13.804 | 1.00 | 33.93 | C |
| ATOM | 3311 | C | ILE | A | 463 | 1.373 | 22.840 | 15.975 | 1.00 | 32.96 | C |
| ATOM | 3312 | O | ILE | A | 463 | 1.438 | 21.719 | 15.489 | 1.00 | 33.12 | O |
| ATOM | 3324 | N | SER | A | 464 | 2.446 | 23.566 | 16.263 | 1.00 | 33.28 | N |
| ATOM | 3325 | CA | SER | A | 464 | 3.793 | 23.143 | 15.893 | 1.00 | 33.50 | C |
| ATOM | 3326 | CB | SER | A | 464 | 4.694 | 24.372 | 15.746 | 1.00 | 33.48 | C |
| ATOM | 3327 | OG | SER | A | 464 | 5.001 | 24.918 | 17.010 | 1.00 | 33.94 | O |
| ATOM | 3328 | C | SER | A | 464 | 4.402 | 22.115 | 16.872 | 1.00 | 33.46 | C |
| ATOM | 3329 | O | SER | A | 464 | 5.169 | 21.261 | 16.462 | 1.00 | 33.02 | O |
| ATOM | 3335 | N | MET | A | 465 | 4.057 | 22.206 | 18.153 | 1.00 | 33.72 | N |
| ATOM | 3336 | CA | MET | A | 465 | 4.394 | 21.162 | 19.116 | 1.00 | 34.13 | C |
| ATOM | 3337 | CB | MET | A | 465 | 4.023 | 21.571 | 20.550 | 1.00 | 34.45 | C |
| ATOM | 3338 | CG | MET | A | 465 | 4.741 | 22.785 | 21.111 | 1.00 | 36.68 | C |
| ATOM | 3339 | SD | MET | A | 465 | 6.544 | 22.667 | 21.073 | 1.00 | 42.74 | S |
| ATOM | 3340 | CE | MET | A | 465 | 6.965 | 23.672 | 19.498 | 1.00 | 40.02 | C |
| ATOM | 3341 | C | MET | A | 465 | 3.634 | 19.877 | 18.780 | 1.00 | 33.70 | C |
| ATOM | 3342 | O | MET | A | 465 | 4.157 | 18.782 | 18.944 | 1.00 | 33.50 | O |
| ATOM | 3352 | N | GLN | A | 466 | 2.386 | 20.022 | 18.349 | 1.00 | 33.59 | N |
| ATOM | 3353 | CA | GLN | A | 466 | 1.560 | 18.882 | 17.964 | 1.00 | 33.51 | C |
| ATOM | 3354 | CB | GLN | A | 466 | 0.119 | 19.313 | 17.662 | 1.00 | 33.76 | C |
| ATOM | 3355 | CG | GLN | A | 466 | −0.759 | 19.653 | 18.877 | 1.00 | 35.20 | C |
| ATOM | 3356 | CD | GLN | A | 466 | −1.920 | 20.597 | 18.503 | 1.00 | 37.64 | C |
| ATOM | 3357 | OE1 | GLN | A | 466 | −2.524 | 20.475 | 17.422 | 1.00 | 37.77 | O |
| ATOM | 3358 | NE2 | GLN | A | 466 | −2.207 | 21.554 | 19.382 | 1.00 | 38.60 | N |
| ATOM | 3359 | C | GLN | A | 466 | 2.157 | 18.233 | 16.723 | 1.00 | 32.95 | C |
| ATOM | 3360 | O | GLN | A | 466 | 2.167 | 17.013 | 16.603 | 1.00 | 32.75 | O |
| ATOM | 3369 | N | ALA | A | 467 | 2.661 | 19.061 | 15.811 | 1.00 | 32.40 | N |
| ATOM | 3370 | CA | ALA | A | 467 | 3.199 | 18.584 | 14.541 | 1.00 | 32.15 | C |
| ATOM | 3371 | CB | ALA | A | 467 | 3.478 | 19.750 | 13.600 | 1.00 | 31.94 | C |
| ATOM | 3372 | C | ALA | A | 467 | 4.463 | 17.778 | 14.767 | 1.00 | 31.81 | C |
| ATOM | 3373 | O | ALA | A | 467 | 4.676 | 16.773 | 14.114 | 1.00 | 31.78 | O |
| ATOM | 3379 | N | GLU | A | 468 | 5.292 | 18.229 | 15.699 | 1.00 | 31.62 | N |
| ATOM | 3380 | CA | GLU | A | 468 | 6.526 | 17.537 | 16.042 | 1.00 | 31.80 | C |
| ATOM | 3381 | CB | GLU | A | 468 | 7.358 | 18.367 | 17.019 | 1.00 | 31.89 | C |
| ATOM | 3382 | CG | GLU | A | 468 | 8.091 | 19.540 | 16.394 | 1.00 | 32.60 | C |
| ATOM | 3383 | CD | GLU | A | 468 | 8.844 | 20.351 | 17.431 | 1.00 | 33.14 | C |
| ATOM | 3384 | OE1 | GLU | A | 468 | 9.472 | 19.757 | 18.320 | 1.00 | 34.44 | O |
| ATOM | 3385 | OE2 | GLU | A | 468 | 8.804 | 21.587 | 17.373 | 1.00 | 35.34 | O |
| ATOM | 3386 | C | GLU | A | 468 | 6.225 | 16.184 | 16.677 | 1.00 | 31.60 | C |
| ATOM | 3387 | O | GLU | A | 468 | 6.913 | 15.205 | 16.419 | 1.00 | 31.23 | O |
| ATOM | 3394 | N | GLU | A | 469 | 5.194 | 16.141 | 17.512 | 1.00 | 31.50 | N |
| ATOM | 3395 | CA | GLU | A | 469 | 4.795 | 14.906 | 18.169 | 1.00 | 31.51 | C |
| ATOM | 3396 | CB | GLU | A | 469 | 3.778 | 15.201 | 19.268 | 1.00 | 31.63 | C |
| ATOM | 3397 | CG | GLU | A | 469 | 4.443 | 15.808 | 20.487 | 1.00 | 32.62 | C |
| ATOM | 3398 | CD | GLU | A | 469 | 3.472 | 16.246 | 21.554 | 1.00 | 33.97 | C |
| ATOM | 3399 | OE1 | GLU | A | 469 | 3.706 | 17.323 | 22.142 | 1.00 | 36.05 | O |
| ATOM | 3400 | OE2 | GLU | A | 469 | 2.492 | 15.519 | 21.820 | 1.00 | 34.65 | O |
| ATOM | 3401 | C | GLU | A | 469 | 4.257 | 13.904 | 17.160 | 1.00 | 31.11 | C |
| ATOM | 3402 | O | GLU | A | 469 | 4.445 | 12.710 | 17.305 | 1.00 | 30.84 | O |
| ATOM | 3409 | N | TYR | A | 470 | 3.615 | 14.421 | 16.124 | 1.00 | 31.18 | N |
| ATOM | 3410 | CA | TYR | A | 470 | 3.097 | 13.624 | 15.029 | 1.00 | 31.08 | C |
| ATOM | 3411 | CB | TYR | A | 470 | 2.157 | 14.479 | 14.181 | 1.00 | 31.08 | C |
| ATOM | 3412 | CG | TYR | A | 470 | 1.884 | 13.895 | 12.826 | 1.00 | 30.75 | C |
| ATOM | 3413 | CD1 | TYR | A | 470 | 1.017 | 12.824 | 12.687 | 1.00 | 29.97 | C |
| ATOM | 3414 | CE1 | TYR | A | 470 | 0.768 | 12.279 | 11.469 | 1.00 | 29.24 | C |
| ATOM | 3415 | CZ | TYR | A | 470 | 1.387 | 12.782 | 10.344 | 1.00 | 29.32 | C |
| ATOM | 3416 | OH | TYR | A | 470 | 1.134 | 12.224 | 9.114 | 1.00 | 29.08 | O |
| ATOM | 3417 | CE2 | TYR | A | 470 | 2.258 | 13.844 | 10.449 | 1.00 | 29.65 | C |
| ATOM | 3418 | CD2 | TYR | A | 470 | 2.507 | 14.392 | 11.689 | 1.00 | 29.63 | C |
| ATOM | 3419 | C | TYR | A | 470 | 4.233 | 13.114 | 14.154 | 1.00 | 31.20 | C |
| ATOM | 3420 | O | TYR | A | 470 | 4.231 | 11.968 | 13.711 | 1.00 | 30.95 | O |
| ATOM | 3430 | N | LEU | A | 471 | 5.202 | 13.986 | 13.906 | 1.00 | 31.47 | N |
| ATOM | 3431 | CA | LEU | A | 471 | 6.342 | 13.670 | 13.055 | 1.00 | 31.45 | C |
| ATOM | 3432 | CB | LEU | A | 471 | 7.090 | 14.957 | 12.711 | 1.00 | 31.58 | C |
| ATOM | 3433 | CG | LEU | A | 471 | 7.955 | 15.021 | 11.454 | 1.00 | 32.71 | C |
| ATOM | 3434 | CD1 | LEU | A | 471 | 9.358 | 14.507 | 11.707 | 1.00 | 33.50 | C |
| ATOM | 3435 | CD2 | LEU | A | 471 | 7.309 | 14.285 | 10.291 | 1.00 | 34.11 | C |
| ATOM | 3436 | C | LEU | A | 471 | 7.273 | 12.674 | 13.752 | 1.00 | 31.18 | C |
| ATOM | 3437 | O | LEU | A | 471 | 7.907 | 11.856 | 13.092 | 1.00 | 30.93 | O |
| ATOM | 3449 | N | TYR | A | 472 | 7.326 | 12.737 | 15.083 | 1.00 | 30.88 | N |
| ATOM | 3450 | CA | TYR | A | 472 | 8.145 | 11.832 | 15.874 | 1.00 | 31.03 | C |
| ATOM | 3451 | CB | TYR | A | 472 | 8.389 | 12.368 | 17.298 | 1.00 | 31.05 | C |
| ATOM | 3452 | CG | TYR | A | 472 | 9.789 | 12.108 | 17.836 | 1.00 | 31.96 | C |
| ATOM | 3453 | CD1 | TYR | A | 472 | 10.508 | 13.112 | 18.492 | 1.00 | 33.47 | C |
| ATOM | 3454 | CE1 | TYR | A | 472 | 11.798 | 12.881 | 18.988 | 1.00 | 33.42 | C |
| ATOM | 3455 | CZ | TYR | A | 472 | 12.371 | 11.633 | 18.829 | 1.00 | 34.10 | C |
| ATOM | 3456 | OH | TYR | A | 472 | 13.632 | 11.382 | 19.304 | 1.00 | 35.26 | O |
| ATOM | 3457 | CE2 | TYR | A | 472 | 11.678 | 10.620 | 18.190 | 1.00 | 34.12 | C |
| ATOM | 3458 | CD2 | TYR | A | 472 | 10.395 | 10.862 | 17.698 | 1.00 | 33.44 | C |
| ATOM | 3459 | C | TYR | A | 472 | 7.461 | 10.472 | 15.928 | 1.00 | 30.86 | C |
| ATOM | 3460 | O | TYR | A | 472 | 8.121 | 9.443 | 15.900 | 1.00 | 30.53 | O |
| ATOM | 3470 | N | TYR | A | 473 | 6.136 | 10.473 | 16.000 | 1.00 | 30.75 | N |
| ATOM | 3471 | CA | TYR | A | 473 | 5.383 | 9.231 | 15.974 | 1.00 | 30.86 | C |
| ATOM | 3472 | CB | TYR | A | 473 | 3.889 | 9.494 | 16.167 | 1.00 | 30.83 | C |
| ATOM | 3473 | CG | TYR | A | 473 | 3.003 | 8.394 | 15.644 | 1.00 | 31.46 | C |
| ATOM | 3474 | CD1 | TYR | A | 473 | 2.811 | 7.223 | 16.372 | 1.00 | 32.50 | C |
| ATOM | 3475 | CE1 | TYR | A | 473 | 2.002 | 6.210 | 15.898 | 1.00 | 32.76 | C |
| ATOM | 3476 | CZ | TYR | A | 473 | 1.378 | 6.361 | 14.679 | 1.00 | 33.12 | C |
| ATOM | 3477 | OH | TYR | A | 473 | 0.570 | 5.366 | 14.196 | 1.00 | 34.13 | O |
| ATOM | 3478 | CE2 | TYR | A | 473 | 1.559 | 7.509 | 13.935 | 1.00 | 32.85 | C |
| ATOM | 3479 | CD2 | TYR | A | 473 | 2.362 | 8.516 | 14.419 | 1.00 | 32.05 | C |
| ATOM | 3480 | C | TYR | A | 473 | 5.649 | 8.502 | 14.655 | 1.00 | 30.79 | C |
| ATOM | 3481 | O | TYR | A | 473 | 5.908 | 7.300 | 14.651 | 1.00 | 30.88 | O |
| ATOM | 3491 | N | LYS | A | 474 | 5.604 | 9.244 | 13.551 | 1.00 | 30.68 | N |
| ATOM | 3492 | CA | LYS | A | 474 | 5.852 | 8.698 | 12.220 | 1.00 | 30.76 | C |
| ATOM | 3493 | CB | LYS | A | 474 | 5.534 | 9.744 | 11.141 | 1.00 | 30.92 | C |
| ATOM | 3494 | CG | LYS | A | 474 | 4.065 | 10.169 | 11.054 | 1.00 | 30.85 | C |
| ATOM | 3495 | CD | LYS | A | 474 | 3.152 | 9.061 | 10.545 | 1.00 | 30.99 | C |
| ATOM | 3496 | CE | LYS | A | 474 | 3.267 | 8.863 | 9.051 | 1.00 | 31.09 | C |
| ATOM | 3497 | NZ | LYS | A | 474 | 2.282 | 7.859 | 8.564 | 1.00 | 31.60 | N |
| ATOM | 3498 | C | LYS | A | 474 | 7.292 | 8.220 | 12.048 | 1.00 | 30.71 | C |
| ATOM | 3499 | O | LYS | A | 474 | 7.532 | 7.242 | 11.358 | 1.00 | 30.68 | O |
| ATOM | 3513 | N | HIS | A | 475 | 8.234 | 8.910 | 12.684 | 1.00 | 30.75 | N |
| ATOM | 3514 | CA | HIS | A | 475 | 9.653 | 8.551 | 12.651 | 1.00 | 30.75 | C |
| ATOM | 3515 | CB | HIS | A | 475 | 10.483 | 9.662 | 13.302 | 1.00 | 30.93 | C |
| ATOM | 3516 | CG | HIS | A | 475 | 11.896 | 9.271 | 13.612 | 1.00 | 31.06 | C |
| ATOM | 3517 | ND1 | HIS | A | 475 | 12.802 | 8.915 | 12.637 | 1.00 | 31.64 | N |
| ATOM | 3518 | CE1 | HIS | A | 475 | 13.960 | 8.621 | 13.199 | 1.00 | 32.02 | C |
| ATOM | 3519 | NE2 | HIS | A | 475 | 13.839 | 8.777 | 14.505 | 1.00 | 32.23 | N |
| ATOM | 3520 | CD2 | HIS | A | 475 | 12.558 | 9.183 | 14.789 | 1.00 | 31.44 | C |
| ATOM | 3521 | C | HIS | A | 475 | 9.935 | 7.231 | 13.365 | 1.00 | 30.67 | C |
| ATOM | 3522 | O | HIS | A | 475 | 10.763 | 6.439 | 12.914 | 1.00 | 30.53 | O |
| ATOM | 3531 | N | LEU | A | 476 | 9.247 | 7.013 | 14.482 | 1.00 | 30.76 | N |
| ATOM | 3532 | CA | LEU | A | 476 | 9.403 | 5.803 | 15.282 | 1.00 | 30.93 | C |
| ATOM | 3533 | CB | LEU | A | 476 | 8.888 | 6.022 | 16.703 | 1.00 | 30.82 | C |
| ATOM | 3534 | CG | LEU | A | 476 | 9.666 | 7.002 | 17.572 | 1.00 | 30.92 | C |
| ATOM | 3535 | CD1 | LEU | A | 476 | 8.931 | 7.201 | 18.879 | 1.00 | 31.19 | C |
| ATOM | 3536 | CD2 | LEU | A | 476 | 11.090 | 6.534 | 17.811 | 1.00 | 31.06 | C |
| ATOM | 3537 | C | LEU | A | 476 | 8.671 | 4.625 | 14.652 | 1.00 | 31.01 | C |
| ATOM | 3538 | O | LEU | A | 476 | 8.976 | 3.477 | 14.958 | 1.00 | 31.05 | O |
| ATOM | 3550 | N | ASN | A | 477 | 7.711 | 4.920 | 13.779 | 1.00 | 31.20 | N |
| ATOM | 3551 | CA | ASN | A | 477 | 6.995 | 3.906 | 13.017 | 1.00 | 31.50 | C |
| ATOM | 3552 | CB | ASN | A | 477 | 5.598 | 4.426 | 12.650 | 1.00 | 31.53 | C |
| ATOM | 3553 | CG | ASN | A | 477 | 4.593 | 3.313 | 12.406 | 1.00 | 31.48 | C |
| ATOM | 3554 | OD1 | ASN | A | 477 | 4.651 | 2.256 | 13.028 | 1.00 | 31.19 | O |
| ATOM | 3555 | ND2 | ASN | A | 477 | 3.651 | 3.557 | 11.502 | 1.00 | 31.87 | N |
| ATOM | 3556 | C | ASN | A | 477 | 7.769 | 3.521 | 11.756 | 1.00 | 31.75 | C |
| ATOM | 3557 | O | ASN | A | 477 | 7.375 | 2.608 | 11.042 | 1.00 | 31.73 | O |
| ATOM | 3564 | N | GLY | A | 478 | 8.862 | 4.235 | 11.483 | 1.00 | 32.20 | N |
| ATOM | 3565 | CA | GLY | A | 478 | 9.733 | 3.951 | 10.355 | 1.00 | 32.35 | C |
| ATOM | 3566 | C | GLY | A | 478 | 9.227 | 4.518 | 9.044 | 1.00 | 32.65 | C |
| ATOM | 3567 | O | GLY | A | 478 | 9.452 | 3.930 | 8.001 | 1.00 | 32.76 | O |
| ATOM | 3571 | N | ASP | A | 479 | 8.560 | 5.667 | 9.094 | 1.00 | 33.17 | N |
| ATOM | 3572 | CA | ASP | A | 479 | 7.935 | 6.270 | 7.911 | 1.00 | 33.61 | C |
| ATOM | 3573 | CB | ASP | A | 479 | 6.507 | 6.721 | 8.233 | 1.00 | 33.59 | C |
| ATOM | 3574 | CG | ASP | A | 479 | 5.576 | 5.564 | 8.523 | 1.00 | 33.30 | C |
| ATOM | 3575 | OD1 | ASP | A | 479 | 5.809 | 4.455 | 7.996 | 1.00 | 32.61 | O |
| ATOM | 3576 | OD2 | ASP | A | 479 | 4.576 | 5.678 | 9.266 | 1.00 | 33.16 | O |
| ATOM | 3577 | C | ASP | A | 479 | 8.706 | 7.471 | 7.368 | 1.00 | 34.18 | C |
| ATOM | 3578 | O | ASP | A | 479 | 8.478 | 7.893 | 6.235 | 1.00 | 34.12 | O |
| ATOM | 3583 | N | VAL | A | 480 | 9.601 | 8.026 | 8.180 | 1.00 | 34.85 | N |
| ATOM | 3584 | CA | VAL | A | 480 | 10.355 | 9.207 | 7.796 | 1.00 | 35.37 | C |
| ATOM | 3585 | CB | VAL | A | 480 | 10.548 | 10.174 | 8.985 | 1.00 | 35.28 | C |
| ATOM | 3586 | CG1 | VAL | A | 480 | 11.118 | 11.490 | 8.506 | 1.00 | 35.36 | C |
| ATOM | 3587 | CG2 | VAL | A | 480 | 9.227 | 10.413 | 9.721 | 1.00 | 35.36 | C |
| ATOM | 3588 | C | VAL | A | 480 | 11.710 | 8.737 | 7.268 | 1.00 | 36.07 | C |
| ATOM | 3589 | O | VAL | A | 480 | 12.563 | 8.325 | 8.059 | 1.00 | 36.27 | O |
| ATOM | 3599 | N | PRO | A | 481 | 11.915 | 8.802 | 5.945 | 1.00 | 36.81 | N |
| ATOM | 3600 | CA | PRO | A | 481 | 13.119 | 8.237 | 5.312 | 1.00 | 37.07 | C |
| ATOM | 3601 | CB | PRO | A | 481 | 12.940 | 8.591 | 3.827 | 1.00 | 37.06 | C |
| ATOM | 3602 | CG | PRO | A | 481 | 11.966 | 9.713 | 3.807 | 1.00 | 37.02 | C |
| ATOM | 3603 | CD | PRO | A | 481 | 11.040 | 9.454 | 4.949 | 1.00 | 36.94 | C |
| ATOM | 3604 | C | PRO | A | 481 | 14.424 | 8.816 | 5.868 | 1.00 | 37.40 | C |
| ATOM | 3605 | O | PRO | A | 481 | 14.435 | 9.956 | 6.341 | 1.00 | 37.33 | O |
| ATOM | 3613 | N | TYR | A | 482 | 15.504 | 8.038 | 5.765 | 1.00 | 37.95 | N |
| ATOM | 3614 | CA | TYR | A | 482 | 16.686 | 8.199 | 6.621 | 1.00 | 38.34 | C |
| ATOM | 3615 | CB | TYR | A | 482 | 17.853 | 7.290 | 6.177 | 1.00 | 38.37 | C |
| ATOM | 3616 | CG | TYR | A | 482 | 19.066 | 7.339 | 7.112 | 1.00 | 38.37 | C |
| ATOM | 3617 | CD1 | TYR | A | 482 | 20.346 | 7.042 | 6.644 | 1.00 | 38.27 | C |
| ATOM | 3618 | CE1 | TYR | A | 482 | 21.460 | 7.088 | 7.498 | 1.00 | 38.25 | C |
| ATOM | 3619 | CZ | TYR | A | 482 | 21.291 | 7.436 | 8.830 | 1.00 | 38.09 | C |
| ATOM | 3620 | OH | TYR | A | 482 | 22.366 | 7.486 | 9.685 | 1.00 | 38.67 | O |
| ATOM | 3621 | CE2 | TYR | A | 482 | 20.036 | 7.734 | 9.318 | 1.00 | 38.49 | C |
| ATOM | 3622 | CD2 | TYR | A | 482 | 18.931 | 7.686 | 8.466 | 1.00 | 38.46 | C |
| ATOM | 3623 | C | TYR | A | 482 | 17.201 | 9.623 | 6.804 | 1.00 | 38.80 | C |
| ATOM | 3624 | O | TYR | A | 482 | 17.946 | 10.142 | 5.976 | 1.00 | 39.04 | O |
| ATOM | 3634 | N | ASN | A | 483 | 16.769 | 10.221 | 7.913 | 1.00 | 39.25 | N |
| ATOM | 3635 | CA | ASN | A | 483 | 17.382 | 11.397 | 8.536 | 1.00 | 39.44 | C |
| ATOM | 3636 | CB | ASN | A | 483 | 18.591 | 10.976 | 9.380 | 1.00 | 39.64 | C |
| ATOM | 3637 | CG | ASN | A | 483 | 18.178 | 10.317 | 10.697 | 1.00 | 40.39 | C |
| ATOM | 3638 | OD1 | ASN | A | 483 | 17.263 | 9.485 | 10.733 | 1.00 | 41.13 | O |
| ATOM | 3639 | ND2 | ASN | A | 483 | 18.839 | 10.704 | 11.786 | 1.00 | 41.32 | N |
| ATOM | 3640 | C | ASN | A | 483 | 17.729 | 12.599 | 7.657 | 1.00 | 39.39 | C |
| ATOM | 3641 | O | ASN | A | 483 | 18.136 | 12.474 | 6.501 | 1.00 | 39.40 | O |
| ATOM | 3648 | N | ASN | A | 484 | 17.557 | 13.773 | 8.260 | 1.00 | 39.11 | N |
| ATOM | 3649 | CA | ASN | A | 484 | 17.561 | 15.034 | 7.545 | 1.00 | 38.80 | C |
| ATOM | 3650 | CB | ASN | A | 484 | 16.279 | 15.144 | 6.701 | 1.00 | 38.99 | C |
| ATOM | 3651 | CG | ASN | A | 484 | 15.164 | 14.220 | 7.185 | 1.00 | 39.62 | C |
| ATOM | 3652 | OD1 | ASN | A | 484 | 14.999 | 13.099 | 6.689 | 1.00 | 39.36 | O |
| ATOM | 3653 | ND2 | ASN | A | 484 | 14.390 | 14.693 | 8.156 | 1.00 | 41.23 | N |
| ATOM | 3654 | C | ASN | A | 484 | 17.681 | 16.211 | 8.527 | 1.00 | 38.46 | C |
| ATOM | 3655 | O | ASN | A | 484 | 17.984 | 16.020 | 9.718 | 1.00 | 38.43 | O |
| ATOM | 3662 | N | LEU | A | 485 | 17.482 | 17.430 | 8.031 | 1.00 | 37.55 | N |
| ATOM | 3663 | CA | LEU | A | 485 | 17.421 | 18.584 | 8.911 | 1.00 | 36.77 | C |
| ATOM | 3664 | CB | LEU | A | 485 | 17.335 | 19.886 | 8.104 | 1.00 | 36.72 | C |
| ATOM | 3665 | CG | LEU | A | 485 | 17.291 | 21.211 | 8.877 | 1.00 | 36.42 | C |
| ATOM | 3666 | CD1 | LEU | A | 485 | 18.450 | 21.318 | 9.878 | 1.00 | 36.10 | C |
| ATOM | 3667 | CD2 | LEU | A | 485 | 17.303 | 22.382 | 7.906 | 1.00 | 36.17 | C |
| ATOM | 3668 | C | LEU | A | 485 | 16.224 | 18.450 | 9.842 | 1.00 | 36.34 | C |
| ATOM | 3669 | O | LEU | A | 485 | 16.311 | 18.822 | 11.002 | 1.00 | 36.44 | O |
| ATOM | 3681 | N | LEU | A | 486 | 15.117 | 17.902 | 9.350 | 1.00 | 35.96 | N |
| ATOM | 3682 | CA | LEU | A | 486 | 13.877 | 17.880 | 10.136 | 1.00 | 35.67 | C |
| ATOM | 3683 | CB | LEU | A | 486 | 12.672 | 17.501 | 9.269 | 1.00 | 35.56 | C |
| ATOM | 3684 | CG | LEU | A | 486 | 11.448 | 18.380 | 9.548 | 1.00 | 35.99 | C |
| ATOM | 3685 | CD1 | LEU | A | 486 | 10.776 | 18.839 | 8.259 | 1.00 | 36.23 | C |
| ATOM | 3686 | CD2 | LEU | A | 486 | 10.461 | 17.642 | 10.439 | 1.00 | 36.77 | C |
| ATOM | 3687 | C | LEU | A | 486 | 13.962 | 16.967 | 11.360 | 1.00 | 35.44 | C |
| ATOM | 3688 | O | LEU | A | 486 | 13.501 | 17.338 | 12.439 | 1.00 | 35.19 | O |
| ATOM | 3700 | N | ILE | A | 487 | 14.567 | 15.793 | 11.190 | 1.00 | 35.33 | N |
| ATOM | 3701 | CA | ILE | A | 487 | 14.730 | 14.831 | 12.280 | 1.00 | 35.36 | C |
| ATOM | 3702 | CB | ILE | A | 487 | 14.969 | 13.391 | 11.715 | 1.00 | 35.46 | C |
| ATOM | 3703 | CG1 | ILE | A | 487 | 13.781 | 12.479 | 12.030 | 1.00 | 35.39 | C |
| ATOM | 3704 | CD1 | ILE | A | 487 | 12.530 | 12.847 | 11.291 | 1.00 | 35.62 | C |
| ATOM | 3705 | CG2 | ILE | A | 487 | 16.236 | 12.749 | 12.268 | 1.00 | 35.75 | C |
| ATOM | 3706 | C | ILE | A | 487 | 15.845 | 15.269 | 13.246 | 1.00 | 35.54 | C |
| ATOM | 3707 | O | ILE | A | 487 | 15.773 | 14.978 | 14.444 | 1.00 | 35.29 | O |
| ATOM | 3719 | N | GLU | A | 488 | 16.860 | 15.969 | 12.734 | 1.00 | 35.70 | N |
| ATOM | 3720 | CA | GLU | A | 488 | 17.903 | 16.534 | 13.595 | 1.00 | 35.93 | C |
| ATOM | 3721 | CB | GLU | A | 488 | 19.098 | 17.061 | 12.796 | 1.00 | 36.07 | C |
| ATOM | 3722 | CG | GLU | A | 488 | 20.361 | 17.164 | 13.648 | 1.00 | 36.89 | C |
| ATOM | 3723 | CD | GLU | A | 488 | 21.522 | 17.848 | 12.944 | 1.00 | 37.36 | C |
| ATOM | 3724 | OE1 | GLU | A | 488 | 21.611 | 19.106 | 13.022 | 1.00 | 36.35 | O |
| ATOM | 3725 | OE2 | GLU | A | 488 | 22.350 | 17.118 | 12.340 | 1.00 | 35.84 | O |
| ATOM | 3726 | C | GLU | A | 488 | 17.370 | 17.644 | 14.491 | 1.00 | 36.07 | C |
| ATOM | 3727 | O | GLU | A | 488 | 17.782 | 17.757 | 15.645 | 1.00 | 36.38 | O |
| ATOM | 3734 | N | MET | A | 489 | 16.464 | 18.467 | 13.971 | 1.00 | 36.15 | N |
| ATOM | 3735 | CA | MET | A | 489 | 15.813 | 19.479 | 14.797 | 1.00 | 36.13 | C |
| ATOM | 3736 | CB | MET | A | 489 | 14.948 | 20.415 | 13.955 | 1.00 | 36.15 | C |
| ATOM | 3737 | CG | MET | A | 489 | 15.660 | 21.161 | 12.840 | 1.00 | 36.56 | C |
| ATOM | 3738 | SD | MET | A | 489 | 17.129 | 22.024 | 13.359 | 1.00 | 37.55 | S |
| ATOM | 3739 | CE | MET | A | 489 | 16.447 | 23.045 | 14.629 | 1.00 | 35.97 | C |
| ATOM | 3740 | C | MET | A | 489 | 14.924 | 18.773 | 15.809 | 1.00 | 36.26 | C |
| ATOM | 3741 | O | MET | A | 489 | 14.868 | 19.155 | 16.979 | 1.00 | 36.69 | O |
| ATOM | 3751 | N | LEU | A | 490 | 14.236 | 17.732 | 15.343 | 1.00 | 36.17 | N |
| ATOM | 3752 | CA | LEU | A | 490 | 13.330 | 16.944 | 16.175 | 1.00 | 36.05 | C |
| ATOM | 3753 | CB | LEU | A | 490 | 12.593 | 15.917 | 15.299 | 1.00 | 36.05 | C |
| ATOM | 3754 | CG | LEU | A | 490 | 11.287 | 15.302 | 15.809 | 1.00 | 35.57 | C |
| ATOM | 3755 | CD1 | LEU | A | 490 | 10.121 | 16.275 | 15.672 | 1.00 | 35.46 | C |
| ATOM | 3756 | CD2 | LEU | A | 490 | 10.992 | 14.014 | 15.059 | 1.00 | 35.60 | C |
| ATOM | 3757 | C | LEU | A | 490 | 14.060 | 16.220 | 17.314 | 1.00 | 36.11 | C |
| ATOM | 3758 | O | LEU | A | 490 | 13.435 | 15.854 | 18.301 | 1.00 | 36.08 | O |
| ATOM | 3770 | N | HIS | A | 491 | 15.373 | 16.024 | 17.168 | 1.00 | 36.38 | N |
| ATOM | 3771 | CA | HIS | A | 491 | 16.173 | 15.234 | 18.110 | 1.00 | 36.54 | C |
| ATOM | 3772 | CB | HIS | A | 491 | 17.446 | 14.698 | 17.431 | 1.00 | 36.60 | C |
| ATOM | 3773 | CG | HIS | A | 491 | 17.294 | 13.341 | 16.805 | 1.00 | 36.62 | C |
| ATOM | 3774 | ND1 | HIS | A | 491 | 16.316 | 12.443 | 17.179 | 1.00 | 36.59 | N |
| ATOM | 3775 | CE1 | HIS | A | 491 | 16.433 | 11.340 | 16.459 | 1.00 | 36.55 | C |
| ATOM | 3776 | NE2 | HIS | A | 491 | 17.454 | 11.488 | 15.633 | 1.00 | 36.46 | N |
| ATOM | 3777 | CD2 | HIS | A | 491 | 18.012 | 12.728 | 15.831 | 1.00 | 36.39 | C |
| ATOM | 3778 | C | HIS | A | 491 | 16.570 | 15.997 | 19.376 | 1.00 | 36.98 | C |
| ATOM | 3779 | O | HIS | A | 491 | 17.134 | 15.399 | 20.291 | 1.00 | 37.55 | O |
| ATOM | 3788 | N | ALA | A | 492 | 16.306 | 17.302 | 19.439 | 1.00 | 37.19 | N |
| ATOM | 3789 | CA | ALA | A | 492 | 16.428 | 18.038 | 20.701 | 1.00 | 37.40 | C |
| ATOM | 3790 | CB | ALA | A | 492 | 17.904 | 18.249 | 21.084 | 1.00 | 37.44 | C |
| ATOM | 3791 | C | ALA | A | 492 | 15.695 | 19.372 | 20.624 | 1.00 | 37.56 | C |
| ATOM | 3792 | O | ALA | A | 492 | 14.466 | 19.402 | 20.521 | 1.00 | 37.90 | O |
| ATOM | 3798 | N | GLU | P | 741 | 26.174 | 18.537 | 18.803 | 1.00 | 34.61 | N |
| ATOM | 3799 | CA | GLU | P | 741 | 24.704 | 18.619 | 19.033 | 1.00 | 34.61 | C |
| ATOM | 3800 | CB | GLU | P | 741 | 24.354 | 19.893 | 19.824 | 1.00 | 34.56 | C |
| ATOM | 3801 | CG | GLU | P | 741 | 23.788 | 19.642 | 21.218 | 1.00 | 34.47 | C |
| ATOM | 3802 | CD | GLU | P | 741 | 22.302 | 19.320 | 21.214 | 1.00 | 34.56 | C |
| ATOM | 3803 | OE1 | GLU | P | 741 | 21.534 | 19.998 | 20.493 | 1.00 | 34.60 | O |
| ATOM | 3804 | OE2 | GLU | P | 741 | 21.895 | 18.391 | 21.944 | 1.00 | 34.22 | O |
| ATOM | 3805 | C | GLU | P | 741 | 23.968 | 18.579 | 17.690 | 1.00 | 34.65 | C |
| ATOM | 3806 | O | GLU | P | 741 | 23.336 | 17.574 | 17.350 | 1.00 | 34.82 | O |
| ATOM | 3812 | N | ASN | P | 742 | 24.075 | 19.665 | 16.928 | 1.00 | 34.52 | N |
| ATOM | 3813 | CA | ASN | P | 742 | 23.393 | 19.807 | 15.648 | 1.00 | 34.44 | C |
| ATOM | 3814 | CS | ASN | P | 742 | 22.328 | 20.908 | 15.750 | 1.00 | 34.59 | C |
| ATOM | 3815 | CG | ASN | P | 742 | 20.926 | 20.358 | 15.920 | 1.00 | 34.84 | C |
| ATOM | 3816 | OD1 | ASN | P | 742 | 20.128 | 20.365 | 14.979 | 1.00 | 34.23 | O |
| ATOM | 3817 | ND2 | ASN | P | 742 | 20.614 | 19.892 | 17.128 | 1.00 | 35.04 | N |
| ATOM | 3818 | C | ASN | P | 742 | 24.400 | 20.152 | 14.557 | 1.00 | 34.22 | C |
| ATOM | 3819 | O | ASN | P | 742 | 24.576 | 21.314 | 14.206 | 1.00 | 34.17 | O |
| ATOM | 3826 | N | ALA | P | 743 | 25.070 | 19.136 | 14.031 | 1.00 | 34.07 | N |
| ATOM | 3827 | CA | ALA | P | 743 | 26.135 | 19.351 | 13.056 | 1.00 | 34.03 | C |
| ATOM | 3828 | CB | ALA | P | 743 | 26.861 | 18.043 | 12.767 | 1.00 | 34.11 | C |
| ATOM | 3829 | C | ALA | P | 743 | 25.615 | 19.974 | 11.759 | 1.00 | 34.04 | C |
| ATOM | 3830 | O | ALA | P | 743 | 26.266 | 20.849 | 11.186 | 1.00 | 34.22 | O |
| ATOM | 3836 | N | LEU | P | 744 | 24.446 | 19.528 | 11.310 | 1.00 | 33.98 | N |
| ATOM | 3837 | CA | LEU | P | 744 | 23.820 | 20.034 | 10.083 | 1.00 | 34.02 | C |
| ATOM | 3838 | CB | LEU | P | 744 | 22.652 | 19.121 | 9.674 | 1.00 | 33.99 | C |
| ATOM | 3839 | CG | LEU | P | 744 | 21.890 | 19.396 | 8.364 | 1.00 | 34.54 | C |
| ATOM | 3840 | CD1 | LEU | P | 744 | 22.805 | 19.843 | 7.223 | 1.00 | 35.40 | C |
| ATOM | 3841 | CD2 | LEU | P | 744 | 21.103 | 18.158 | 7.935 | 1.00 | 34.32 | C |
| ATOM | 3842 | C | LEU | P | 744 | 23.340 | 21.491 | 10.209 | 1.00 | 33.95 | C |
| ATOM | 3843 | O | LEU | P | 744 | 23.545 | 22.292 | 9.299 | 1.00 | 34.11 | O |
| ATOM | 3855 | N | LEU | P | 745 | 22.705 | 21.831 | 11.330 | 1.00 | 33.80 | N |
| ATOM | 3856 | CA | LEU | P | 745 | 22.260 | 23.207 | 11.581 | 1.00 | 33.70 | C |
| ATOM | 3857 | CB | LEU | P | 745 | 21.507 | 23.314 | 12.923 | 1.00 | 33.70 | C |
| ATOM | 3858 | CG | LEU | P | 745 | 20.266 | 24.207 | 13.046 | 1.00 | 33.27 | C |
| ATOM | 3859 | CD1 | LEU | P | 745 | 20.170 | 24.815 | 14.437 | 1.00 | 32.24 | C |
| ATOM | 3860 | CD2 | LEU | P | 745 | 20.186 | 25.299 | 11.975 | 1.00 | 33.44 | C |
| ATOM | 3861 | C | LEU | P | 745 | 23.450 | 24.157 | 11.605 | 1.00 | 33.73 | C |
| ATOM | 3862 | O | LEU | P | 745 | 23.385 | 25.264 | 11.077 | 1.00 | 34.09 | O |
| ATOM | 3874 | N | ARG | P | 746 | 24.535 | 23.721 | 12.232 | 1.00 | 33.50 | N |
| ATOM | 3875 | CA | ARG | P | 746 | 25.749 | 24.518 | 12.295 | 1.00 | 33.42 | C |
| ATOM | 3876 | CB | ARG | P | 746 | 26.766 | 23.850 | 13.219 | 1.00 | 33.49 | C |
| ATOM | 3877 | CG | ARG | P | 746 | 28.025 | 24.669 | 13.417 | 1.00 | 33.46 | C |
| ATOM | 3878 | CD | ARG | P | 746 | 28.982 | 24.111 | 14.442 | 1.00 | 33.87 | C |
| ATOM | 3879 | NE | ARG | P | 746 | 29.480 | 25.184 | 15.296 | 1.00 | 35.04 | N |
| ATOM | 3880 | CZ | ARG | P | 746 | 28.927 | 25.572 | 16.444 | 1.00 | 35.37 | C |
| ATOM | 3881 | NH1 | ARG | P | 746 | 27.846 | 24.968 | 16.930 | 1.00 | 35.42 | N |
| ATOM | 3882 | NH2 | ARG | P | 746 | 29.475 | 26.576 | 17.121 | 1.00 | 36.26 | N |
| ATOM | 3883 | C | ARG | P | 746 | 26.366 | 24.736 | 10.907 | 1.00 | 33.26 | C |
| ATOM | 3884 | O | ARG | P | 746 | 26.813 | 25.836 | 10.583 | 1.00 | 33.19 | O |
| ATOM | 3898 | N | TYR | P | 747 | 26.396 | 23.683 | 10.099 | 1.00 | 33.16 | N |
| ATOM | 3899 | CA | TYR | P | 747 | 26.945 | 23.768 | 8.753 | 1.00 | 33.21 | C |
| ATOM | 3900 | CB | TYR | P | 747 | 26.883 | 22.404 | 8.051 | 1.00 | 33.28 | C |
| ATOM | 3901 | CG | TYR | P | 747 | 27.324 | 22.460 | 6.599 | 1.00 | 33.04 | C |
| ATOM | 3902 | CD1 | TYR | P | 747 | 28.666 | 22.570 | 6.272 | 1.00 | 32.68 | C |
| ATOM | 3903 | CE1 | TYR | P | 747 | 29.081 | 22.640 | 4.951 | 1.00 | 32.93 | C |
| ATOM | 3904 | CZ | TYR | P | 747 | 28.148 | 22.613 | 3.936 | 1.00 | 32.17 | C |
| ATOM | 3905 | OH | TYR | P | 747 | 28.574 | 22.695 | 2.636 | 1.00 | 30.38 | O |
| ATOM | 3906 | CE2 | TYR | P | 747 | 26.803 | 22.519 | 4.234 | 1.00 | 32.58 | C |
| ATOM | 3907 | CD2 | TYR | P | 747 | 26.396 | 22.444 | 5.562 | 1.00 | 32.25 | C |
| ATOM | 3908 | C | TYR | P | 747 | 26.196 | 24.812 | 7.924 | 1.00 | 33.22 | C |
| ATOM | 3909 | O | TYR | P | 747 | 26.809 | 25.580 | 7.185 | 1.00 | 32.89 | O |
| ATOM | 3919 | N | LEU | P | 748 | 24.874 | 24.830 | 8.058 | 1.00 | 33.41 | N |
| ATOM | 3920 | CA | LEU | P | 748 | 24.022 | 25.713 | 7.273 | 1.00 | 33.75 | C |
| ATOM | 3921 | CB | LEU | P | 748 | 22.569 | 25.242 | 7.338 | 1.00 | 33.68 | C |
| ATOM | 3922 | CG | LEU | P | 748 | 22.337 | 23.852 | 6.737 | 1.00 | 34.49 | C |
| ATOM | 3923 | CD1 | LEU | P | 748 | 21.048 | 23.231 | 7.267 | 1.00 | 35.23 | C |
| ATOM | 3924 | CD2 | LEU | P | 748 | 22.312 | 23.899 | 5.219 | 1.00 | 34.49 | C |
| ATOM | 3925 | C | LEU | P | 748 | 24.131 | 27.167 | 7.729 | 1.00 | 34.01 | C |
| ATOM | 3926 | O | LEU | P | 748 | 23.866 | 28.078 | 6.951 | 1.00 | 34.17 | O |
| ATOM | 3938 | N | LEU | P | 749 | 24.534 | 27.379 | 8.978 | 1.00 | 34.31 | N |
| ATOM | 3939 | CA | LEU | P | 749 | 24.711 | 28.727 | 9.518 | 1.00 | 34.77 | C |
| ATOM | 3940 | CB | LEU | P | 749 | 24.395 | 28.734 | 11.018 | 1.00 | 34.88 | C |
| ATOM | 3941 | CG | LEU | P | 749 | 22.940 | 28.447 | 11.393 | 1.00 | 34.51 | C |
| ATOM | 3942 | CD1 | LEU | P | 749 | 22.849 | 28.088 | 12.868 | 1.00 | 34.55 | C |
| ATOM | 3943 | CD2 | LEU | P | 749 | 22.055 | 29.637 | 11.075 | 1.00 | 34.15 | C |
| ATOM | 3944 | C | LEU | P | 749 | 26.107 | 29.341 | 9.284 | 1.00 | 35.01 | C |
| ATOM | 3945 | O | LEU | P | 749 | 26.273 | 30.550 | 9.453 | 1.00 | 34.78 | O |
| ATOM | 3957 | N | ASP | P | 750 | 27.089 | 28.517 | 8.901 | 1.00 | 35.61 | N |
| ATOM | 3958 | CA | ASP | P | 750 | 28.480 | 28.965 | 8.668 | 1.00 | 35.93 | C |
| ATOM | 3959 | CB | ASP | P | 750 | 29.477 | 27.962 | 9.256 | 1.00 | 36.06 | C |
| ATOM | 3960 | CG | ASP | P | 750 | 29.436 | 27.913 | 10.768 | 1.00 | 36.98 | C |
| ATOM | 3961 | OD1 | ASP | P | 750 | 29.449 | 28.987 | 11.411 | 1.00 | 36.82 | O |
| ATOM | 3962 | OD2 | ASP | P | 750 | 29.397 | 26.837 | 11.399 | 1.00 | 38.78 | O |
| ATOM | 3963 | C | ASP | P | 750 | 28.816 | 29.154 | 7.185 | 1.00 | 35.85 | C |
| ATOM | 3964 | O | ASP | P | 750 | 29.368 | 30.184 | 6.799 | 1.00 | 36.02 | O |
| ATOM | 3969 | N | LYS | P | 751 | 28.522 | 28.140 | 6.374 | 1.00 | 35.65 | N |
| ATOM | 3970 | CA | LYS | P | 751 | 28.734 | 28.191 | 4.924 | 1.00 | 35.57 | C |
| ATOM | 3971 | CB | LYS | P | 751 | 30.206 | 28.463 | 4.586 | 1.00 | 35.59 | C |
| ATOM | 3972 | CG | LYS | P | 751 | 30.463 | 29.776 | 3.854 | 1.00 | 35.24 | C |
| ATOM | 3973 | CD | LYS | P | 751 | 31.916 | 30.205 | 3.997 | 1.00 | 34.64 | C |
| ATOM | 3974 | CE | LYS | P | 751 | 32.268 | 31.306 | 3.020 | 1.00 | 34.57 | C |
| ATOM | 3975 | NZ | LYS | P | 751 | 31.359 | 32.471 | 3.156 | 1.00 | 34.62 | N |
| ATOM | 3976 | C | LYS | P | 751 | 28.309 | 26.871 | 4.273 | 1.00 | 35.69 | C |
| ATOM | 3977 | O | LYS | P | 751 | 27.211 | 26.366 | 4.514 | 1.00 | 35.79 | O |
| ATOM | 3991 | N | ASN | Q | 742 | 6.446 | 8.836 | −7.386 | 1.00 | 33.84 | N |
| ATOM | 3992 | CA | ASN | Q | 742 | 5.438 | 8.085 | −8.179 | 1.00 | 33.97 | C |
| ATOM | 3993 | CB | ASN | O | 742 | 5.294 | 6.647 | −7.653 | 1.00 | 34.00 | C |
| ATOM | 3994 | CG | ASN | Q | 742 | 6.317 | 5.690 | −8.262 | 1.00 | 34.60 | C |
| ATOM | 3995 | OD1 | ASN | Q | 742 | 6.298 | 5.429 | −9.467 | 1.00 | 35.17 | O |
| ATOM | 3996 | ND2 | ASN | Q | 742 | 7.212 | 5.159 | −7.427 | 1.00 | 34.86 | N |
| ATOM | 3997 | C | ASN | Q | 742 | 4.085 | 8.813 | −8.208 | 1.00 | 34.03 | C |
| ATOM | 3998 | O | ASN | Q | 742 | 3.900 | 9.725 | −9.013 | 1.00 | 34.29 | O |
| ATOM | 4004 | N | ALA | Q | 743 | 3.149 | 8.442 | −7.332 | 1.00 | 33.87 | N |
| ATOM | 4005 | CA | ALA | Q | 743 | 1.750 | 8.853 | −7.499 | 1.00 | 33.63 | C |
| ATOM | 4006 | CB | ALA | Q | 743 | 0.838 | 8.046 | −6.589 | 1.00 | 33.61 | C |
| ATOM | 4007 | C | ALA | Q | 743 | 1.549 | 10.344 | −7.263 | 1.00 | 33.66 | C |
| ATOM | 4008 | O | ALA | Q | 743 | 1.095 | 11.052 | −8.159 | 1.00 | 33.60 | O |
| ATOM | 4014 | N | LEU | Q | 744 | 1.902 | 10.815 | −6.067 | 1.00 | 33.62 | N |
| ATOM | 4015 | CA | LEU | Q | 744 | 1.717 | 12.220 | −5.699 | 1.00 | 33.50 | C |
| ATOM | 4016 | CB | LEU | Q | 744 | 2.040 | 12.445 | −4.218 | 1.00 | 33.52 | C |
| ATOM | 4017 | CG | LEU | Q | 744 | 1.892 | 13.887 | −3.710 | 1.00 | 33.80 | C |
| ATOM | 4018 | CD1 | LEU | Q | 744 | 0.518 | 14.458 | −4.037 | 1.00 | 34.25 | C |
| ATOM | 4019 | CD2 | LEU | Q | 744 | 2.139 | 13.963 | −2.216 | 1.00 | 33.73 | C |
| ATOM | 4020 | C | LEU | Q | 744 | 2.550 | 13.173 | −6.545 | 1.00 | 33.35 | C |
| ATOM | 4021 | O | LEU | Q | 744 | 2.045 | 14.196 | −6.998 | 1.00 | 33.25 | O |
| ATOM | 4033 | N | LEU | Q | 745 | 3.824 | 12.846 | −6.744 | 1.00 | 33.30 | N |
| ATOM | 4034 | CA | LEU | Q | 745 | 4.725 | 13.714 | −7.502 | 1.00 | 33.28 | C |
| ATOM | 4035 | CB | LEU | Q | 745 | 6.149 | 13.151 | −7.488 | 1.00 | 33.29 | C |
| ATOM | 4036 | CG | LEU | Q | 745 | 7.251 | 14.015 | −8.111 | 1.00 | 33.71 | C |
| ATOM | 4037 | CD1 | LEU | Q | 745 | 7.542 | 15.243 | −7.264 | 1.00 | 33.90 | C |
| ATOM | 4038 | CD2 | LEU | Q | 745 | 8.520 | 13.200 | −8.308 | 1.00 | 33.82 | C |
| ATOM | 4039 | C | LEU | Q | 745 | 4.235 | 13.912 | −8.944 | 1.00 | 33.20 | C |
| ATOM | 4040 | O | LEU | Q | 745 | 4.297 | 15.018 | −9.472 | 1.00 | 33.21 | O |
| ATOM | 4052 | N | ARG | Q | 746 | 3.741 | 12.842 | −9.563 | 1.00 | 33.07 | N |
| ATOM | 4053 | CA | ARG | Q | 746 | 3.205 | 12.906 | −10.923 | 1.00 | 33.12 | C |
| ATOM | 4054 | CB | ARG | Q | 746 | 2.917 | 11.499 | −11.464 | 1.00 | 33.14 | C |
| ATOM | 4055 | CG | ARG | Q | 746 | 2.375 | 11.499 | −12.884 | 1.00 | 33.02 | C |
| ATOM | 4056 | CD | ARG | Q | 746 | 2.622 | 10.222 | −13.663 | 1.00 | 33.71 | C |
| ATOM | 4057 | NE | ARG | Q | 746 | 2.144 | 10.353 | −15.043 | 1.00 | 33.83 | N |
| ATOM | 4058 | CZ | ARG | Q | 746 | 1.975 | 9.348 | −15.895 | 1.00 | 33.75 | C |
| ATOM | 4059 | NH1 | ARG | Q | 746 | 2.250 | 8.095 | −15.543 | 1.00 | 34.27 | N |
| ATOM | 4060 | NH2 | ARG | Q | 746 | 1.525 | 9.597 | −17.117 | 1.00 | 34.03 | N |
| ATOM | 4061 | C | ARG | Q | 746 | 1.936 | 13.760 | −10.995 | 1.00 | 33.14 | C |
| ATOM | 4062 | O | ARG | Q | 746 | 1.723 | 14.493 | −11.960 | 1.00 | 33.03 | O |
| ATOM | 4076 | N | TYR | Q | 747 | 1.100 | 13.656 | −9.968 | 1.00 | 33.26 | N |
| ATOM | 4077 | CA | TYR | Q | 747 | −0.101 | 14.473 | −9.849 | 1.00 | 33.28 | C |
| ATOM | 4078 | CB | TYR | Q | 747 | −0.900 | 14.050 | −8.611 | 1.00 | 33.30 | C |
| ATOM | 4079 | CG | TYR | Q | 747 | −1.943 | 15.048 | −8.171 | 1.00 | 33.77 | C |
| ATOM | 4080 | CD1 | TYR | Q | 747 | −1.782 | 15.782 | −6.998 | 1.00 | 34.13 | C |
| ATOM | 4081 | CE1 | TYR | Q | 747 | −2.744 | 16.708 | −6.593 | 1.00 | 34.84 | C |
| ATOM | 4082 | CZ | TYR | Q | 747 | −3.882 | 16.904 | −7.372 | 1.00 | 34.56 | C |
| ATOM | 4083 | OH | TYR | Q | 747 | −4.838 | 17.814 | −6.990 | 1.00 | 34.21 | O |
| ATOM | 4084 | CE2 | TYR | Q | 747 | −4.056 | 16.187 | −8.541 | 1.00 | 34.53 | C |
| ATOM | 4085 | CD2 | TYR | Q | 747 | −3.090 | 15.266 | −8.934 | 1.00 | 34.20 | C |
| ATOM | 4086 | C | TYR | Q | 747 | 0.229 | 15.969 | −9.794 | 1.00 | 33.27 | C |
| ATOM | 4087 | O | TYR | Q | 747 | −0.511 | 16.790 | −10.341 | 1.00 | 33.15 | O |
| ATOM | 4097 | N | LEU | Q | 748 | 1.345 | 16.313 | −9.150 | 1.00 | 33.36 | N |
| ATOM | 4098 | CA | LEU | Q | 748 | 1.741 | 17.712 | −8.976 | 1.00 | 33.37 | C |
| ATOM | 4099 | CB | LEU | Q | 748 | 2.834 | 17.832 | −7.909 | 1.00 | 33.40 | C |
| ATOM | 4100 | CG | LEU | Q | 748 | 2.445 | 17.435 | −6.478 | 1.00 | 33.31 | C |
| ATOM | 4101 | CD1 | LEU | Q | 748 | 3.686 | 17.318 | −5.594 | 1.00 | 33.15 | C |
| ATOM | 4102 | CD2 | LEU | Q | 748 | 1.444 | 18.411 | −5.863 | 1.00 | 33.23 | C |
| ATOM | 4103 | C | LEU | Q | 748 | 2.206 | 18.346 | −10.287 | 1.00 | 33.44 | C |
| ATOM | 4104 | O | LEU | Q | 748 | 1.816 | 19.467 | −10.615 | 1.00 | 33.35 | O |
| ATOM | 4116 | N | LEU | Q | 749 | 3.022 | 17.615 | −11.038 | 1.00 | 33.66 | N |
| ATOM | 4117 | CA | LEU | Q | 749 | 3.526 | 18.091 | −12.327 | 1.00 | 33.97 | C |
| ATOM | 4118 | CB | LEU | Q | 749 | 4.519 | 17.086 | −12.918 | 1.00 | 34.02 | C |
| ATOM | 4119 | CG | LEU | Q | 749 | 5.759 | 16.725 | −12.095 | 1.00 | 34.34 | C |
| ATOM | 4120 | CD1 | LEU | Q | 749 | 6.403 | 15.454 | −12.650 | 1.00 | 34.83 | C |
| ATOM | 4121 | CD2 | LEU | Q | 749 | 6.751 | 17.879 | −12.076 | 1.00 | 34.34 | C |
| ATOM | 4122 | C | LEU | Q | 749 | 2.397 | 18.305 | −13.339 | 1.00 | 34.10 | C |
| ATOM | 4123 | O | LEU | Q | 749 | 2.336 | 19.345 | −14.001 | 1.00 | 34.12 | O |
| ATOM | 4135 | N | ASP | Q | 750 | 1.504 | 17.320 | −13.437 | 1.00 | 34.17 | N |
| ATOM | 4136 | CA | ASP | Q | 750 | 0.480 | 17.283 | −14.484 | 1.00 | 34.24 | C |
| ATOM | 4137 | CB | ASP | Q | 750 | −0.186 | 15.896 | −14.528 | 1.00 | 34.24 | C |
| ATOM | 4138 | CG | ASP | Q | 750 | 0.696 | 14.833 | −15.193 | 1.00 | 34.67 | C |
| ATOM | 4139 | OD1 | ASP | Q | 750 | 1.827 | 15.155 | −15.631 | 1.00 | 34.76 | O |
| ATOM | 4140 | OD2 | ASP | Q | 750 | 0.334 | 13.641 | −15.325 | 1.00 | 34.79 | O |
| ATOM | 4141 | C | ASP | Q | 750 | −0.586 | 18.387 | −14.376 | 1.00 | 34.20 | C |
| ATOM | 4142 | O | ASP | Q | 750 | −1.326 | 18.620 | −15.335 | 1.00 | 34.27 | O |
| ATOM | 4147 | N | LYS | Q | 751 | −0.669 | 19.053 | −13.222 | 1.00 | 34.12 | N |
| ATOM | 4148 | CA | LYS | Q | 751 | −1.497 | 20.253 | −13.068 | 1.00 | 34.04 | C |
| ATOM | 4149 | CB | LYS | Q | 751 | −2.928 | 19.877 | −12.652 | 1.00 | 34.03 | C |
| ATOM | 4150 | CG | LYS | Q | 751 | −3.966 | 19.942 | −13.766 | 1.00 | 33.85 | C |
| ATOM | 4151 | CD | LYS | Q | 751 | −4.345 | 21.375 | −14.117 | 1.00 | 33.62 | C |
| ATOM | 4152 | CE | LYS | Q | 751 | −5.318 | 21.422 | −15.292 | 1.00 | 33.41 | C |
| ATOM | 4153 | NZ | LYS | Q | 751 | −5.054 | 22.579 | −16.185 | 1.00 | 33.02 | N |
| ATOM | 4154 | C | LYS | Q | 751 | −0.881 | 21.198 | −12.031 | 1.00 | 34.07 | C |
| ATOM | 4155 | O | LYS | Q | 751 | 0.328 | 21.448 | −12.031 | 1.00 | 33.89 | O |
| ATOM | 4169 | O43 | PPA | L | 1 | −2.683 | 13.046 | 2.647 | 1.00 | 36.59 | O |
| ATOM | 4170 | C42 | PPA | L | 1 | −1.973 | 12.439 | 1.856 | 1.00 | 36.77 | C |
| ATOM | 4171 | C44 | PPA | L | 1 | −1.315 | 13.116 | 0.665 | 1.00 | 36.61 | C |
| ATOM | 4172 | C45 | PPA | L | 1 | −1.556 | 14.627 | 0.599 | 1.00 | 36.00 | C |
| ATOM | 4173 | C46 | PPA | L | 1 | −0.460 | 15.423 | 1.301 | 1.00 | 35.69 | C |
| ATOM | 4174 | C47 | PPA | L | 1 | −0.890 | 16.866 | 1.574 | 1.00 | 35.51 | C |
| ATOM | 4175 | C48 | PPA | L | 1 | 0.176 | 17.655 | 2.340 | 1.00 | 35.35 | C |
| ATOM | 4176 | C49 | PPA | L | 1 | −0.440 | 18.673 | 3.301 | 1.00 | 35.77 | C |
| ATOM | 4177 | C50 | PPA | L | 1 | 0.569 | 19.206 | 4.325 | 1.00 | 35.36 | C |
| ATOM | 4178 | C51 | PPA | L | 1 | 1.309 | 20.425 | 3.796 | 1.00 | 35.59 | C |
| ATOM | 4179 | C52 | PPA | L | 1 | 2.761 | 20.483 | 4.283 | 1.00 | 36.70 | C |
| ATOM | 4180 | C53 | PPA | L | 1 | 2.893 | 20.954 | 5.734 | 1.00 | 36.61 | C |
| ATOM | 4181 | C54 | PPA | L | 1 | 4.043 | 21.942 | 5.875 | 1.00 | 36.48 | C |
| ATOM | 4182 | C55 | PPA | L | 1 | 4.143 | 22.465 | 7.304 | 1.00 | 37.07 | C |
| ATOM | 4183 | C56 | PPA | L | 1 | 4.833 | 21.460 | 8.196 | 1.00 | 37.56 | C |
| ATOM | 4184 | C57 | PPA | L | 1 | 5.276 | 21.837 | 9.396 | 1.00 | 38.55 | C |
| ATOM | 4185 | C58 | PPA | L | 1 | 5.976 | 20.859 | 10.319 | 1.00 | 38.57 | C |
| ATOM | 4186 | O41 | PPA | L | 1 | −1.752 | 11.011 | 2.077 | 1.00 | 36.88 | O |
| ATOM | 4187 | C40 | PPA | L | 1 | −1.735 | 10.466 | 3.404 | 1.00 | 35.73 | C |
| ATOM | 4188 | C38 | PPA | L | 1 | −0.454 | 10.840 | 4.147 | 1.00 | 34.28 | C |
| ATOM | 4189 | C39 | PPA | L | 1 | −0.291 | 9.981 | 5.398 | 1.00 | 33.72 | C |
| ATOM | 4190 | O19 | PPA | L | 1 | −1.199 | 10.305 | 6.457 | 1.00 | 33.32 | O |
| ATOM | 4191 | P16 | PPA | L | 1 | −1.011 | 9.490 | 7.839 | 1.00 | 33.40 | P |
| ATOM | 4192 | O17 | PPA | L | 1 | 0.404 | 9.747 | 8.301 | 1.00 | 32.80 | O |
| ATOM | 4193 | O18 | PPA | L | 1 | −1.470 | 8.066 | 7.620 | 1.00 | 34.54 | O |
| ATOM | 4194 | O15 | PPA | L | 1 | −2.015 | 10.119 | 8.930 | 1.00 | 34.72 | O |
| ATOM | 4195 | C14 | PPA | L | 1 | −1.556 | 10.312 | 10.276 | 1.00 | 35.34 | C |
| ATOM | 4196 | C6 | PPA | L | 1 | −2.191 | 9.386 | 11.302 | 1.00 | 35.84 | C |
| ATOM | 4197 | O7 | PPA | L | 1 | −3.227 | 10.071 | 11.968 | 1.00 | 35.93 | O |
| ATOM | 4198 | C8 | PPA | L | 1 | −1.182 | 8.964 | 12.355 | 1.00 | 36.16 | C |
| ATOM | 4199 | O9 | PPA | L | 1 | −1.615 | 7.735 | 12.931 | 1.00 | 37.73 | O |
| ATOM | 4200 | P10 | PPA | L | 1 | −2.873 | 7.663 | 13.933 | 1.00 | 39.45 | P |
| ATOM | 4201 | O12 | PPA | L | 1 | −4.116 | 7.289 | 13.150 | 1.00 | 40.20 | O |
| ATOM | 4202 | O13 | PPA | L | 1 | −2.848 | 8.894 | 14.792 | 1.00 | 38.10 | O |
| ATOM | 4203 | O11 | PPA | L | 1 | −2.556 | 6.354 | 14.835 | 1.00 | 39.26 | O |
| ATOM | 4204 | C1 | PPA | L | 1 | −2.997 | 6.260 | 16.190 | 1.00 | 37.98 | C |
| ATOM | 4205 | C2 | PPA | L | 1 | −4.455 | 5.829 | 16.286 | 1.00 | 37.45 | C |
| ATOM | 4206 | C4 | PPA | L | 1 | −5.119 | 6.606 | 17.415 | 1.00 | 37.20 | C |
| ATOM | 4207 | O5 | PPA | L | 1 | −5.436 | 7.911 | 16.978 | 1.00 | 36.01 | O |
| ATOM | 4208 | O3 | PPA | L | 1 | −4.558 | 4.439 | 16.569 | 1.00 | 36.89 | O |
| ATOM | 4209 | O37 | PPA | L | 1 | −0.493 | 12.216 | 4.510 | 1.00 | 34.58 | O |
| ATOM | 4210 | C21 | PPA | L | 1 | 0.800 | 12.858 | 4.656 | 1.00 | 34.98 | C |
| ATOM | 4211 | O20 | PPA | L | 1 | 1.663 | 12.665 | 3.821 | 1.00 | 35.77 | O |
| ATOM | 4212 | C22 | PPA | L | 1 | 1.064 | 13.763 | 5.837 | 1.00 | 35.70 | C |
| ATOM | 4213 | C23 | PPA | L | 1 | 1.171 | 15.234 | 5.433 | 1.00 | 36.25 | C |
| ATOM | 4214 | C24 | PPA | L | 1 | 2.597 | 15.624 | 5.056 | 1.00 | 36.54 | C |
| ATOM | 4215 | C25 | PPA | L | 1 | 3.562 | 15.418 | 6.214 | 1.00 | 36.61 | C |
| ATOM | 4216 | C26 | PPA | L | 1 | 4.644 | 16.488 | 6.265 | 1.00 | 37.07 | C |
| ATOM | 4217 | C27 | PPA | L | 1 | 5.594 | 16.193 | 7.426 | 1.00 | 37.44 | C |
| ATOM | 4218 | C28 | PPA | L | 1 | 6.185 | 17.459 | 8.035 | 1.00 | 37.93 | C |
| ATOM | 4219 | C29 | PPA | L | 1 | 5.940 | 17.528 | 9.527 | 1.00 | 38.45 | C |
| ATOM | 4220 | C30 | PPA | L | 1 | 4.730 | 17.746 | 10.054 | 1.00 | 39.11 | C |
| ATOM | 4221 | C31 | PPA | L | 1 | 3.494 | 17.929 | 9.200 | 1.00 | 38.75 | C |
| ATOM | 4222 | C32 | PPA | L | 1 | 2.382 | 18.600 | 10.002 | 1.00 | 37.88 | C |
| ATOM | 4223 | C33 | PPA | L | 1 | 1.194 | 18.898 | 9.087 | 1.00 | 37.14 | C |
| ATOM | 4224 | C34 | PPA | L | 1 | 0.081 | 19.598 | 9.852 | 1.00 | 36.55 | C |
| ATOM | 4225 | C35 | PPA | L | 1 | 0.284 | 21.107 | 9.890 | 1.00 | 35.37 | C |
| ATOM | 4226 | C36 | PPA | L | 1 | −0.314 | 21.757 | 8.667 | 1.00 | 34.63 | C |
| ATOM | 4227 | O3 | TRS | L | 3 | 9.347 | 29.715 | 2.255 | 1.00 | 42.27 | O |
| ATOM | 4228 | C3 | TRS | L | 3 | 10.479 | 30.376 | 2.803 | 1.00 | 41.84 | C |
| ATOM | 4229 | C | TRS | L | 3 | 10.156 | 31.810 | 3.223 | 1.00 | 41.88 | C |
| ATOM | 4230 | N | TRS | L | 3 | 9.040 | 31.825 | 4.173 | 1.00 | 42.35 | N |
| ATOM | 4231 | C2 | TRS | L | 3 | 11.367 | 32.385 | 3.945 | 1.00 | 41.73 | C |
| ATOM | 4232 | O2 | TRS | L | 3 | 11.291 | 32.060 | 5.319 | 1.00 | 41.98 | O |
| ATOM | 4233 | C1 | TRS | L | 3 | 9.766 | 32.667 | 2.012 | 1.00 | 42.24 | C |
| ATOM | 4234 | O1 | TRS | L | 3 | 8.431 | 32.437 | 1.595 | 1.00 | 42.95 | O |
| ATOM | 4235 | O | HOH | S | 1 | 2.108 | 5.986 | −1.133 | 1.00 | 23.43 | O |
| ATOM | 4236 | O | HOH | S | 2 | 13.244 | 5.029 | 13.018 | 1.00 | 35.88 | O |
| ATOM | 4237 | O | HOH | S | 3 | 11.511 | 38.773 | 6.589 | 1.00 | 30.96 | O |
| ATOM | 4238 | O | HOH | S | 4 | 15.542 | 30.877 | 1.405 | 1.00 | 42.07 | O |
| ATOM | 4239 | O | HOH | S | 5 | 13.286 | 31.186 | 18.972 | 1.00 | 25.09 | O |
| ATOM | 4240 | O | HOH | S | 6 | 0.792 | 27.561 | 22.768 | 1.00 | 32.79 | O |
| ATOM | 4241 | O | HOH | S | 7 | −5.956 | 26.103 | 15.642 | 1.00 | 35.97 | O |
| ATOM | 4242 | O | HOH | S | 8 | 24.892 | 33.395 | 10.244 | 1.00 | 29.28 | O |
| ATOM | 4243 | O | HOH | S | 9 | 6.109 | 25.566 | 5.485 | 1.00 | 31.19 | O |
| ATOM | 4244 | O | HOH | S | 10 | 15.560 | 15.611 | 2.255 | 1.00 | 33.67 | O |
| ATOM | 4245 | O | HOH | S | 11 | 8.926 | 31.294 | 19.207 | 1.00 | 38.26 | O |
| ATOM | 4246 | O | HOH | S | 12 | −7.554 | 15.507 | 7.142 | 1.00 | 32.33 | O |
| ATOM | 4247 | O | HOH | S | 13 | −10.744 | 29.028 | 1.079 | 1.00 | 35.19 | O |
| ATOM | 4248 | O | HOH | S | 14 | −11.785 | 42.259 | 12.347 | 1.00 | 25.32 | O |
| ATOM | 4249 | O | HOH | S | 15 | 9.213 | 25.519 | 16.927 | 1.00 | 31.32 | O |
| ATOM | 4250 | O | HOH | S | 16 | −4.149 | 23.203 | 18.085 | 1.00 | 36.87 | O |
| ATOM | 4251 | O | HOH | S | 17 | −9.056 | 18.745 | 19.148 | 1.00 | 32.61 | O |
| ATOM | 4252 | O | HOH | S | 18 | 4.012 | 22.577 | 1.516 | 1.00 | 31.64 | O |
| ATOM | 4253 | O | HOH | S | 19 | −2.446 | 3.229 | 17.583 | 1.00 | 41.97 | O |
| ATOM | 4254 | O | HOH | S | 20 | 0.638 | 36.935 | 22.612 | 1.00 | 45.48 | O |
| ATOM | 4255 | O | HOH | S | 21 | 18.151 | 17.304 | 4.963 | 1.00 | 33.50 | O |
| ATOM | 4256 | O | HOH | S | 22 | −12.356 | 38.189 | 17.814 | 1.00 | 34.96 | O |
| ATOM | 4257 | O | HOH | S | 23 | 0.105 | 41.383 | 1.512 | 1.00 | 32.51 | O |
| ATOM | 4258 | O | HOH | S | 24 | 25.510 | 40.496 | 25.615 | 1.00 | 30.99 | O |
| ATOM | 4259 | O | HOH | S | 25 | 29.679 | 33.213 | 15.194 | 1.00 | 31.76 | O |
| ATOM | 4260 | O | HOH | S | 26 | 9.168 | 30.436 | 22.604 | 1.00 | 40.62 | O |
| ATOM | 4261 | O | HOH | S | 27 | 23.696 | 15.780 | 14.185 | 1.00 | 36.32 | O |
| ATOM | 4262 | O | HOH | S | 28 | −4.140 | 26.248 | 17.617 | 1.00 | 38.61 | O |
| ATOM | 4263 | O | HOH | S | 29 | −5.299 | 7.378 | −3.270 | 1.00 | 28.27 | O |
| ATOM | 4264 | O | HOH | S | 30 | −6.455 | 6.396 | 13.074 | 1.00 | 39.28 | O |
| ATOM | 4265 | O | HOH | S | 31 | 21.631 | 15.274 | 16.032 | 1.00 | 30.87 | O |
| ATOM | 4266 | O | HOH | S | 32 | 21.836 | 28.385 | 5.120 | 1.00 | 22.13 | O |
| ATOM | 4267 | O | HOH | S | 33 | 29.257 | 21.528 | 11.551 | 1.00 | 26.18 | O |
| ATOM | 4268 | O | HOH | S | 34 | −2.361 | 18.935 | −9.788 | 1.00 | 25.42 | O |
| ATOM | 4271 | O | HOH | S | 37 | −0.094 | 5.428 | 10.256 | 1.00 | 20.40 | O |

TABLE 4
Human SF-1 amino acid and cDNA nucleotide sequences.

Sequence NM_004959

| | | | | | | | |
|---|---|---|---|---|---|---|---|
| 1 | ggaggacgga | cggacagggc | cagcctgctg | tccggctgcc | gcccgccgtg | gtgtgagggg | (SEQ ID NO:_) |
| 61 | gtttctgcgc | acccacagtc | gccaccgtcc | cacctgggct | gccggagcct | ccccctggac | |
| 121 | ccctggtgcc | cactgccacc | ctcatccggt | gtgagagcgc | tgcttccgct | tcgcggacgc | |
| 181 | cgcgggcatg | gactattcgt | acgacgagga | cctggacgag | ctgtgccccg | tgtgcgggga | |
| 241 | caaggtgtcc | ggctaccact | acggactgct | cacgtgtgag | agctgcaagg | gcttcttcaa | |
| 301 | gcgcacggtg | cagaacaaca | agoactacac | gtgcaccgag | agccagagct | gcaagatcga | |
| 361 | caagacgcag | cgcaagcgct | gtcccttctg | ccgcttccag | aaatgcctga | cggtggggat | |
| 421 | gcgcctggaa | gccgtgcgcg | ctgaccgtat | gaggggtggc | cggaacaagt | ttgggccgat | |
| 481 | gtacaagcgg | gaccgggccc | tgaaacagca | gaagaaggca | cagattcggg | ccaatggctt | |
| 541 | caagctggag | acagggcccc | cgatgggggt | gcccccgccg | ccccctcccg | caccggacta | |
| 601 | cgtgctgcct | cccagcctgc | atgggcctga | gcccaagggc | ctggccgccg | gtccacctgc | |
| 661 | tgggccactg | ggcgactttg | gggccccagc | actgcccatg | gccgtgcccg | gtgcccacgg | |
| 721 | gccactggct | ggctacctct | accctgcctt | tcctggccgt | gccatcaagt | ctgagtaccc | |
| 781 | ggagccttat | gccagccccc | cacagcctgg | gctgccgtac | ggctacccag | agcccttctc | |
| 841 | tggagggccc | aacgtgcctg | agctcatcct | gcagctgctg | cagctggagc | cggatgagga | |
| 901 | ccaggtgcgg | gcccgcatct | tgggctgcct | gcaggagccc | accaaaagcc | gccccgacca | |
| 961 | gccggcggcc | ttcggcctcc | tgtgcagaat | ggccgaccag | accttcatct | ccatcgtgga | |
| 1021 | ctgggcacgc | aggtgcatgg | tcttcaagga | gctggaggtg | gccgaccaga | tgacgctgct | |
| 1081 | gcagaactgc | tggagcgagc | tgctggtgtt | cgaccacatc | taccgccagg | tccagcacgg | |
| 1141 | caaggagggc | agcatcctgc | tggtcaccgg | gcaggaggtg | gagctgacca | cagtggccac | |
| 1201 | ccaggcgggc | tcgctgctgc | acagcctggt | gttgcgggcg | caggagctgg | tgctgcagct | |
| 1261 | gcttgcgctg | cagctggacc | ggcaggagtt | tgtctgcctc | aagttcatca | tcctcttcag | |
| 1321 | cctggatttg | aagttcctga | ataaccacat | cctggtgaaa | gacgctcagg | agaaggccaa | |
| 1381 | cgccgccctg | cttgactaca | ccctgtgcca | ctacccgcac | tgcggggaca | aattccagca | |
| 1441 | gctgctgctg | tgcctggtgg | aggtgcgggc | cctgagcatg | caggccaagg | agtacctgta | |
| 1501 | ccacaagcac | ctgggcaacg | agatgccccg | caacaacctg | ctcatcgaaa | tgctgcaagc | |
| 1561 | caagcagact | tgagcctggg | ccgggggcgg | ggccgggact | gggggcggga | ctgggggcgg | |
| 1621 | ggcctgggcg | gggccgcagc | cacaccgctg | gctctgcatg | gttcattttc | tgatgcccac | |
| 1681 | cgaggagccc | cagccccgtc | ccagaggccg | ctgcccctga | gttctgacac | tgtgtgtttg | |
| 1741 | ggaagtgggt | gaggctgggc | agggcctggc | ggaggtggag | tggccactgg | cacttgcctg | |
| 1801 | ctgcttggag | tgccccaagg | aggtggctgt | taaccacccg | ccccgccccc | tccctgctcc | |
| 1861 | cagctctctc | tcctggagtc | tgaagcctgc | aggtccgggg | aggaggttcg | ggattccctg | |
| 1921 | gtgggcctcg | acgtcccttg | gatcagaggt | catcccttcc | tcctctcctg | gaaacagaca | |
| 1981 | gggagaagtt | gagcaggtat | caactagggg | aggagagagg | gtctccagtg | ttccccccat | |
| 2041 | agagaccagg | agggagagcc | tctgttttgt | aaactaagga | taaccgagtt | tgctaaattg | |
| 2101 | agaggggcta | ttgggcccta | gaggacacta | ggagactggt | taggacaaaa | agaccttctc | |
| 2161 | cctagccctt | ctaccccacc | tgacctctgc | aagagggggc | attgatacat | catcgggaaa | |
| 2221 | aaactttgct | ccaggcatca | ctgattccct | ctcccaccca | aggagaacgt | ttggtacaat | |
| 2281 | cgacatccta | gccccaccca | gaggtggccc | tcccaggctg | gtatttatct | gcaaggttgt | |
| 2341 | agtcaagagg | tttttctccc | cgctttttgt | ttttaagctt | ctagacactc | cttgaaatgt | |
| 2401 | gtgtgtgatg | gagggaaggg | gacagatttg | aggactgaag | ctggggcttg | gggattgcca | |
| 2461 | ctaagtacag | ctgatggttt | ctccccggac | actcgcctac | taagtaccct | tggggtggtg | |
| 2521 | ctgggtcatt | acttctgagc | cccagcccca | atccagagaa | gcgctgttgc | ccgccctcca | |
| 2581 | cccactaggt | gaacagcagg | atgccctgtt | gggggcttca | ggtctctgtg | ggtgggaatg | |
| 2641 | caagtgaact | tgggaggggg | cacgggcctg | tagatcaggg | atagcgctgt | tgatcccctc | |
| 2701 | tctgtggctc | caacccgttg | ggtcccttgc | tgcaaaccca | tgaagctggc | cctcagctcc | |
| 2761 | ctgaccccct | gtcctaggtc | atgaaggaca | ctctgcaggg | tgaagcacca | gggagaggcc | |
| 2821 | tcggctgtct | cctgtccccg | gcggggtgcc | tgctgtccgt | cccgctttca | tgttactgtt | |
| 2881 | gcagcttgtg | ctgagcctgc | ccagttggag | gagactgggc | acccctgcct | cctgcctccc | |
| 2941 | gcctcccgcc | accctgtctc | agtacctccc | ccccccgccc | cctgaaacat | gtgcccctgc | |
| 3001 | caaggccgga | gacccacagc | cctgaaacga | gaagtgccct | taaggatcac | cccagccccc | |
| 3061 | acagccctgg | aataaatttc | gcaattagtt | tccaaaaaaa | aaaaaaaaaa | aaaaaaaaa | |

Sequence NP_004950

| | | | | | | | |
|---|---|---|---|---|---|---|---|
| 1 | mdysydedld | elcpvcgdkv | sgyhyglltc | esckgffkrt | vqnnkhytct | esqsckidkt | (SEQ ID NO:_) |
| 61 | qrkrcpfcrf | qkcltvgmrl | eavradrmrg | grnkfgpmyk | rdralkqqkk | aqirangfkl | |
| 121 | etgppmgvpp | ppppapdyvl | ppslhgpepk | glaagppagp | lgdfgapalp | mavpgahgpl | |
| 181 | agylypafpg | raikseypep | yasppqpglp | ygypepfsgg | pnvpelilql | lqlepdedqv | |
| 241 | rarilgclqe | ptksrpdqpa | afgllcrmad | qtfisivdwa | rrcmvfkele | vadqmtllqn | |
| 301 | cwsellvfdh | iyrqvqhgke | gsillvtgqe | velttvatqa | gsllhslvlr | aqelvlqlla | |
| 361 | lqldrqefvc | lkfiilfsld | lkflnnhilv | kdaqekanaa | lidytlchyp | hcgdkfqqll | |
| 421 | lclvevrals | mqakeylyhk | hlgnemprnn | lliemlqakq | t | | |

TABLE 5

Human LRH-1 amino acid and cDNA nucleotide sequences, and mouse LRH-1
nucleotide sequence.

Sequence NM_003822

| | | | | | | | |
|---|---|---|---|---|---|---|---|
| 1 | aaaaagtaca | gagtccaggg | aaagacttgc | ttgtaacttt | atgaattctg | gatttttttt | (SEQ ID NO:_) |
| 61 | tttcctttgc | tttttcttaa | ctttcactaa | gggttactgt | agtctgatgt | gtccttccca | |
| 121 | aggccacgaa | atttgacaag | ctgcactttt | cttttgctca | atgatttctg | ctttaagcca | |
| 181 | aagaactgcc | tataatttca | ctaagaatgt | cttctaattc | agatactggg | gatttacaag | |
| 241 | agtctttaaa | gcacggactt | acacctattg | tgtctcaatt | taaaatggtg | aattactcct | |
| 301 | atgatgaaga | tctggaagag | ctttgtcccg | tgtgtggaga | taaagtgtct | gggtaccatt | |
| 361 | atgggctcct | cacctgtgaa | agctgcaagg | gattttttaa | gcgaacagtc | caaaataata | |
| 421 | aaaggtacac | atgtatagaa | aaccagaact | gccaaattga | caaaacacag | agaaagcgtt | |
| 481 | gtccttactg | tcgttttcaa | aaatgtctaa | gtgttggaat | gaagctagaa | gctgtaaggg | |
| 541 | ccgaccgaat | gcgtggagga | aggaataagt | ttgggccaat | gtacaagaga | gacagggccc | |
| 601 | tgaagcaaca | gaaaaaagcc | ctcatccgag | ccaatggact | taagctagaa | gccatgtctc | |
| 661 | aggtgatcca | agctatgccc | tctgacctga | ccatttcctc | tgcaattcaa | aacatccact | |
| 721 | ctgcctccaa | aggcctacct | ctgaaccatg | ctgccttgcc | tcctacagac | tatgacagaa | |
| 781 | gtccctttgt | aacatccccc | attagcatga | caatgccccc | tcacggcagc | ctgcaaggtt | |
| 841 | accaaacata | tggccacttt | cctagccggg | ccatcaagtc | tgagtaccca | gacccctata | |
| 901 | ccagctcacc | cgagtccata | atgggctatt | catatatgga | tagttaccag | acgagctctc | |
| 961 | cagcaagcat | cccacatctg | atactggaac | ttttgaagtg | tgagccagat | gagcctcaag | |
| 1021 | tccaggctaa | aatcatggcc | tatttgcagc | aagagcaggc | taaccgaagc | aagcacgaaa | |
| 1081 | agctgagcac | ctttgggctt | atgtgcaaaa | tggcagatca | aactctcttc | tccattgtcg | |
| 1141 | agtgggccag | gagtagtatc | ttcttcagag | aacttaaggt | tgatgaccaa | atgaagctgc | |
| 1201 | ttcagaactg | ctggagtgag | ctcttaatcc | tcgaccacat | ttaccgacaa | gtggtacatg | |
| 1261 | gaaaggaagg | atccatcttc | ctggttactg | ggcaacaagt | ggactattcc | ataatagcat | |
| 1321 | cacaagccgg | agccaccctc | aacaacctca | tgagtcatgc | acaggagtta | gtggcaaaac | |
| 1381 | ttcgttctct | ccagtttgat | caacgagagt | tcgtatgtct | gaaattcttg | gtgctcttta | |
| 1441 | gtttagatgt | caaaaacctt | gaaaacttcc | agctggtaga | aggtgtccag | gaacaagtca | |
| 1501 | atgccgccct | gctggactac | acaatgtgta | actacccgca | gcagacagag | aaatttggac | |
| 1561 | agctacttct | tcgactaccc | gaaatccggg | ccatcagtat | gcaggctgaa | gaatacctct | |
| 1621 | actacaagca | cctgaacggg | gatgtgccct | ataataacct | tctcattgaa | atgttgcatg | |
| 1681 | ccaaaagagc | ataagttaca | acccctagga | gctctgcttt | caaaacaaaa | agagattggg | |
| 1741 | ggagtgggga | gggggaagaa | gaacaggaag | aaaaaaagta | ctctgaactg | ctccaagtaa | |
| 1801 | cgctaattaa | aaacttgctt | taaagatatt | gaatttaaaa | aggcataata | atcaaatact | |
| 1861 | taatagcaaa | taaatgatgt | atcagggtat | ttgtattgca | aactgtgaat | caaaggcttc | |
| 1921 | acagccccag | aggattccat | ataaaagaca | ttgtaatgga | gtggattgaa | ctcacagatg | |
| 1981 | gataccaaca | cggtcagaag | aaaaacggac | agaacggttc | ttgtatattt | aaactgatct | |
| 2041 | ccactatgaa | gaaatttagg | aactaatctt | attaattagg | cttatacagc | gggggatttg | |
| 2101 | agcttacagg | attcctccat | ggtaaagctg | aactgaaaca | attctcaaga | atgcatcagc | |
| 2161 | tgtacctaca | atagcccctc | cctcttcctt | tgaaggcccc | agcacctctg | ccctgtggtc | |
| 2221 | accgaatctg | tactaaggac | ctgtgttcag | ccacacccag | tggtagctcc | accaaatcat | |
| 2281 | gaacagccta | attttgagtg | tctgtgtctt | agacctgcaa | acagctaata | ggaaattcta | |
| 2341 | ttaatatgtt | agcttgccat | tttaaatatg | ttctgagggt | tgttttgtct | cgtgttcatg | |
| 2401 | atgttaagaa | aatgcaggca | gtatccctca | tcttatgtaa | gtgtgaatta | atattaaggg | |
| 2461 | aaatgactac | aaactttcaa | agcaaatgct | ccatagctaa | agcaacttag | accttatttc | |
| 2521 | tgctactgtt | gctgaaatgt | ggctttggca | ttgttggatt | tcataaaaaa | tttctggcag | |
| 2581 | gaagtcttgt | tagtatacat | cagtcttttt | catcatccaa | gtttgtagtt | catttaaaaa | |
| 2641 | tacaacatta | aacacatttt | gctaggatgt | caaatagtca | cagttctaag | tagttggaaa | |
| 2701 | caaaattgac | gcatgttaat | ctatgcaaag | agaaaggaaa | ggatgaggtg | atgtattgac | |
| 2761 | tcaaggttca | ttcttgctgc | aattgaacat | cctcaagagt | tgggatggaa | atggtgattt | |
| 2821 | ttacatgtgt | cctggaaaga | tattaaagta | attcaaatct | tccccaaagg | ggaaaggaag | |
| 2881 | agagtgatac | tgaccttttt | aagtcataga | ccaaagtctg | ctgtagaaca | aatatgggag | |
| 2941 | gacaaagaat | cgcaaattct | tcaaatgact | attatcagta | ttattaacat | gcgatgccac | |
| 3001 | aggtatgaaa | gtcttgcctt | atttcacaat | tttaaaaggt | agctgtgcag | atgtggatca | |
| 3061 | acatttgttt | aaaataaagt | attaatactt | taaagtcaaa | taagatatag | tgtttacatt | |
| 3121 | ctttaggtcc | tgaggggcag | ggggatctgt | gatataacaa | aatagcaaaa | gcggtaattt | |
| 3181 | ccttaatgtt | atttttctga | ttggtaatta | tttttaacag | tacttaatta | ttctatgtcg | |
| 3241 | tgagacacta | aaatcaaaaa | cgggaatctc | atttagactt | taattttttt | gagattatcg | |
| 3301 | gcggcacaat | cactttgtag | aaactgtaaa | aaataaaagt | atctcctagt | cccttaattt | |
| 3361 | tttcataaat | atttctggct | tttgagtagt | gtatttatat | tgtatatcat | actttcaact | |
| 3421 | gtagacaatt | atgatgctaa | tttattgttt | cttggtttca | cctttgtata | agatatagcc | |
| 3481 | aagactgaag | aaaccaaata | tatgtgttta | ctgtagcatg | tcttcaaatt | agtggaactt | |
| 3541 | agttcaggga | catagaagag | tcttaatgaa | ttaaaatcat | tcacttgatt | aaatgtctgt | |
| 3601 | aaatcttcat | cattcctact | gtagtttatt | taatatctat | tgtaaattat | gtgacttgta | |
| 3661 | gcttcctctg | gttttcaagt | aaactcaaca | aggtggagtc | ttacctggtt | ttcctttcca | |
| 3721 | agcattgtaa | attgtatacc | aaagatatta | gttattactt | ctgtgtgtac | aaagaggatt | |
| 3781 | attttattat | gtttattaat | cacctctaat | actcatccac | atgaagggta | cacattaggt | |
| 3841 | aagctgggcg | ttgactcatg | cgcagtctca | gtcacccgtg | ttatcttcgt | ggctcaaagg | |
| 3901 | acaatgcaaa | atcgccgatc | agagctcata | cccaaagcat | tacagagaac | agcagcatca | |
| 3961 | ttgccctccc | cagctgaaaa | acaagttggc | tagaagatac | atggagagga | atggtgtggt | |
| 4021 | caacagttaa | tgaaacggtt | ctatcatgca | tgtgtaatgt | ggatggagac | aattataaga | |
| 4081 | tttgactata | actatttgga | gggtctttaa | cattgccaaa | aaaacaaata | tgttgatttt | |
| 4141 | tattttattt | tattttttat | tttaagaggc | gggatcttga | tctcacatgt | tgcccaggct | |
| 4201 | ggccttgaac | tcctgggctc | aagcattcct | cctgcctcag | cctcccccat | agctgggact | |
| 4261 | aggggtgcat | gccagcatac | ctggctacgt | tgactcttaa | aatctatgtt | ctcttatttt | |
| 4321 | aaagatacag | tgctccccac | tgaaaattaa | acctaaaaaa | tgtcacatat | tggtatgttg | |
| 4381 | ttaacctggt | agattaaatc | atgagaatga | ttagaaagac | gggcaacaca | gcgggttaca | |
| 4441 | tccacactgc | tgatcacacc | aacgacagga | gctgataagc | aagaaagcgt | cacagccagc | |
| 4501 | gtctgttcac | ccaaggttga | caagtgaagt | ttctctaatg | ttgattgtta | gccgatttgt | |
| 4561 | aacctggcat | ttacttagca | actgccttat | caattacagg | atttgccggt | aaaagcagac | |
| 4621 | tcaaatataa | aggtttttgg | cttaacttgg | tttattatag | ttgctctatg | tttgtaaaca | |
| 4681 | gacaatctct | aatgtctgat | tatttgtatc | acagatctgc | agctgccttg | gacttgaatc | |
| 4741 | catgcaatgt | ttagagtgtg | aagtcagtta | cttgttgatg | ttttcttact | gtatcaatga | |
| 4801 | aatacatatt | gtcatgtcag | ttcttgccag | gaacttctca | acaaaatgga | attttttttt | |
| 4861 | tcagtatttc | aataaatatt | gatatgccca | gcctgataat | ttttaaaaaa | aaaaaa | |

Sequence NP_003813

| | | | | | | | |
|---|---|---|---|---|---|---|---|
| 1 | mssnsdtgdl | qeslkhgltp | ivsqfkmvny | sydedleelc | pvcgdkvsgy | hyglltcesc | (SEQ ID NO:_) |
| 61 | kgffkrtvqn | nkrytcienq | ncqidktqrk | rcpycrfqkc | lsvgmkleav | radrmrggrn | |
| 121 | kfgpmykrdr | alkqqkkali | ranglkleam | sqviqampsd | ltissaiqni | hsaskglpln | |
| 181 | haalpptdyd | rspfvtspis | mtmpphgslq | gyqtyghfps | raikseypdp | ytsspesimg | |
| 241 | ysymdsyqts | spasiphlil | ellkcepdep | qvqakimayl | qqeqanrskh | eklstfglmc | |
| 301 | kmadqtlfsi | vewarssiff | relkvddqmk | llqncwsell | ildhiyrqvv | hgkegsiflv | |
| 361 | tgqqvdysii | asqagatlnn | lmshaqelva | klrslqfdqr | efvclkflvl | fsldvknlen | |
| 421 | fqlvegvqeq | vnaalldytm | cnypqqtekf | gqlllrlpei | raismqaeey | lyykhlngdv | |
| 481 | pynnllieml | hakra | | | | | |

Sequence NM_205860

| | | | | | | | |
|---|---|---|---|---|---|---|---|
| 1 | aaaaagtaca | gagtccaggg | aaagacttgc | ttgtaacttt | atgaattctg | gatttttttt | (SEQ ID NO:_) |
| 61 | tttcctttgc | tttttcttaa | ctttcactaa | gggttactgt | agtctgatgt | gtccttccca | |
| 121 | aggccacgaa | atttgacaag | ctgcactttt | cttttgctca | atgatttctg | ctttaagcca | |
| 181 | aagaactgcc | tataatttca | ctaagaatgt | cttctaattc | agatactggg | gatttacaag | |
| 241 | agtctttaaa | gcacggactt | acacctattg | gtgctgggct | tccggaccga | cacggatccc | |
| 301 | ccatccccgc | ccgcggtcgc | cttgtcatgc | tgcccaaagt | ggagacggaa | gccctgggac | |
| 361 | tggctcgatc | gcatggggaa | cagggccaga | tgccggaaaa | catgcaagtg | tctcaattta | |
| 421 | aaatggtgaa | ttactcctat | gatgaagatc | tggaagagct | ttgtcccgtg | tgtggagata | |
| 481 | aagtgtctgg | gtaccattat | gggctcctca | cctgtgaaag | ctgcaaggga | ttttttaagc | |
| 541 | gaacagtcca | aaataataaa | aggtacacat | gtatagaaaa | ccagaactgc | caaattgaca | |
| 601 | aaacacagag | aaagcgttgt | ccttactgtc | gttttcaaaa | atgtctaagt | gttggaatga | |
| 661 | agctagaagc | tgtaagggcc | gaccgaatgc | gtggaggaag | gaataagttt | gggccaatgt | |
| 721 | acaagagaga | cagggccctg | aagcaacaga | aaaaagccct | catccgagcc | aatggactta | |
| 781 | agctagaagc | catgtctcag | gtgatccaag | ctatgccctc | tgacctgacc | atttcctctg | |
| 841 | caattcaaaa | catccactct | gcctccaaag | gcctacctct | gaaccatgct | gccttgcctc | |
| 901 | ctacagacta | tgacagaagt | ccctttgtaa | catcccccat | tagcatgaca | atgccccctc | |
| 961 | acggcagcct | gcaaggttac | caaacatatg | gccactttcc | tagccgggcc | atcaagtctg | |
| 1021 | agtacccaga | cccctatacc | agctcacccg | agtccataat | gggctattca | tatatggata | |
| 1081 | gttaccagac | gagctctcca | gcaagcatcc | cacatctgat | actggaactt | ttgaagtgtg | |
| 1141 | agccagatga | gcctcaagtc | caggctaaaa | tcatggccta | tttgcagcaa | gagcaggcta | |
| 1201 | accgaagcaa | gcacgaaaag | ctgagcacct | ttgggcttat | gtgcaaaatg | gcagatcaaa | |
| 1261 | ctctcttctc | cattgtcgag | tgggccagga | gtagtatctt | cttcagagaa | cttaaggttg | |
| 1321 | atgaccaaat | gaagctgctt | cagaactgct | ggagtgagct | cttaatcctc | gaccacattt | |
| 1381 | accgacaagt | ggtacatgga | aaggaaggat | ccatcttcct | ggttactggg | caacaagtgg | |
| 1441 | actattccat | aatagcatca | caagccggag | ccaccctcaa | caacctcatg | agtcatgcac | |
| 1501 | aggagttagt | ggcaaaactt | cgttctctcc | agtttgatca | acgagagttc | gtatgtctga | |
| 1561 | aattcttggt | gctctttagt | ttagatgtca | aaaaccttga | aaacttccag | ctggtagaag | |
| 1621 | gtgtccagga | acaagtcaat | gccgccctgc | tggactacac | aatgtgtaac | tacccgcagc | |
| 1681 | agacagagaa | atttggacag | ctacttcttc | gactacccga | aatccgggcc | atcagtatgc | |
| 1741 | aggctgaaga | atacctctac | tacaagcacc | tgaacgggga | tgtgccctat | aataaccttc | |
| 1801 | tcattgaaat | gttgcatgcc | aaaagagcat | aagttacaac | ccctaggagc | tctgctttca | |
| 1861 | aaacaaaaag | agattggggg | agtggggagg | gggaagaaga | acaggaagaa | aaaaagtact | |
| 1921 | ctgaactgct | ccaagtaacg | ctaattaaaa | acttgcttta | aagatattga | atttaaaaag | |
| 1981 | gcataataat | caaatactta | atagcaaata | aatgatgtat | cagggtattt | gtattgcaaa | |
| 2041 | ctgtgaatca | aaggcttcac | agccccagag | gattccatat | aaaagacatt | gtaatggagt | |
| 2101 | ggattgaact | cacagatgga | taccaacacg | gtcagaagaa | aaacggacag | aacggttctt | |
| 2161 | gtatatttaa | actgatctcc | actatgaaga | aatttaggaa | ctaatcttat | taattaggct | |
| 2221 | tatacagcgg | gggatttgag | cttacaggat | tcctccatgg | taaagctgaa | ctgaaacaat | |
| 2281 | tctcaagaat | gcatcagctg | tacctacaat | agcccctccc | tcttcctttg | aaggccccag | |
| 2341 | cacctctgcc | ctgtggtcac | cgaatctgta | ctaaggacct | gtgttcagcc | acacccagtg | |
| 2401 | gtagctccac | caaatcatga | acagcctaat | tttgagtgtc | tgtgtcttag | acctgcaaac | |
| 2461 | agctaatagg | aaattctatt | aatatgttag | cttgccattt | taaatatgtt | ctgagggttg | |
| 2521 | ttttgtctcg | tgttcatgat | gttaagaaaa | tgcaggcagt | atccctcatc | ttatgtaagt | |
| 2581 | gtgaattaat | attaagggaa | atgactacaa | actttcaaag | caaatgctcc | atagctaaag | |
| 2641 | caacttagac | cttatttctg | ctactgttgc | tgaaatgtgg | ctttggcatt | gttggatttc | |
| 2701 | ataaaaaatt | tctggcagga | agtcttgtta | gtatacatca | gtctttttca | tcatccaagt | |
| 2761 | ttgtagttca | tttaaaaata | caacattaaa | cacattttgc | taggatgtca | aatagtcaca | |
| 2821 | gttctaagta | gttggaaaca | aaattgacgc | atgttaatct | atgcaaagag | aaaggaaagg | |
| 2881 | atgaggtgat | gtattgactc | aaggttcatt | cttgctgcaa | ttgaacatcc | tcaagagttg | |
| 2941 | ggatggaaat | ggtgattttt | acatgtgtcc | tggaaagata | ttaaagtaat | tcaaatcttc | |
| 3001 | cccaaagggg | aaaggaagag | agtgatactg | acctttttaa | gtcatagacc | aaagtctgct | |
| 3061 | gtagaacaaa | tatgggagga | caaagaatcg | caaattcttc | aaatgactat | tatcagtatt | |
| 3121 | attaacatgc | gatgccacag | gtatgaaagt | cttgccttat | ttcacaattt | taaaaggtag | |
| 3181 | ctgtgcagat | gtggatcaac | atttgtttaa | aataaagtat | taatacttta | aagtcaaata | |
| 3241 | agatatagtg | tttacattct | ttaggtcctg | aggggcaggg | ggatctgtga | tataacaaaa | |
| 3301 | tagcaaaagc | ggtaatttcc | ttaatgttat | ttttctgatt | ggtaattatt | tttaacagta | |
| 3361 | cttaattatt | ctatgtcgtg | agacactaaa | atcaaaaacg | ggaatctcat | ttagacttta | |
| 3421 | atttttttga | gattatcggc | ggcacaatca | ctttgtagaa | actgtaaaaa | ataaaagtat | |
| 3481 | ctcctagtcc | cttaattttt | tcataaatat | ttctggcttt | tgagtagtgt | atttatattg | |
| 3541 | tatatcatac | tttcaactgt | agacaattat | gatgctaatt | tattgtttct | tggtttcacc | |
| 3601 | tttgtataag | atatagccaa | gactgaagaa | accaaatata | tgtgtttact | gtagcatgtc | |
| 3661 | ttcaaattag | tggaacttag | ttcagggaca | tagaagagtc | ttaatgaatt | aaaatcattc | |
| 3721 | acttgattaa | atgtctgtaa | atcttcatca | ttcctactgt | agtttattta | atatctattg | |
| 3781 | taaattatgt | gacttgtagc | ttcctctggt | tttcaagtaa | actcaacaag | gtggagtctt | |
| 3841 | acctggtttt | cctttccaag | cattgtaaat | tgtataccaa | agatattagt | tattacttct | |
| 3901 | gtgtgtacaa | agaggattat | tttattatgt | ttattaatca | cctctaatac | tcatccacat | |
| 3961 | gaagggtaca | cattaggtaa | gctgggcgtt | gactcatgcg | cagtctcagt | cacccgtgtt | |
| 4021 | atcttcgtgg | ctcaaaggac | aatgcaaaat | cgccgatcag | agctcatacc | caaagcatta | |
| 4081 | cagagaacag | cagcatcatt | gccctcccca | gctgaaaaac | aagttggcta | gaagatacat | |
| 4141 | ggagaggaat | ggtgtggtca | acagttaatg | aaacggttct | atcatgcatg | tgtaatgtgg | |
| 4201 | atggagacaa | ttataagatt | tgactataac | tatttggagg | gtctttaaca | ttgccaaaaa | |
| 4261 | aacaaatatg | ttgattttta | ttttatttta | ttttttattt | taagaggcgg | gatcttgatc | |
| 4321 | tcacatgttg | cccaggctgg | ccttgaactc | ctgggctcaa | gcattcctcc | tgcctcagcc | |
| 4381 | tcccccatag | ctgggactag | gggtgcatgc | cagcatacct | ggctacgttg | actcttaaaa | |
| 4441 | tctatgttct | cttattttaa | agatacagtg | ctccccactg | aaaattaaac | ctaaaaaatg | |
| 4501 | tcacatattg | gtatgttgtt | aacctggtag | attaaatcat | gagaatgatt | agaaagacgg | |
| 4561 | gcaacacagc | gggttacatc | cacactgctg | atcacaccaa | cgacaggagc | tgataagcaa | |
| 4621 | gaaagcgtca | cagccagcgt | ctgttcaccc | aaggttgaca | agtgaagttt | ctctaatgtt | |
| 4681 | gattgttagc | cgatttgtaa | cctggcattt | acttagcaac | tgccttatca | attacaggat | |
| 4741 | ttgccggtaa | aagcagactc | aaatataaag | gtttttggct | taacttggtt | tattatagtt | |
| 4801 | gctctatgtt | tgtaaacaga | caatctctaa | tgtctgatta | tttgtatcac | agatctgcag | |
| 4861 | ctgccttgga | cttgaatcca | tgcaatgttt | agagtgtgaa | gtcagttact | tgttgatgtt | |
| 4921 | ttcttactgt | atcaatgaaa | tacatattgt | catgtcagtt | cttgccagga | acttctcaac | |
| 4981 | aaaatggaat | tttttttttc | agtatttcaa | taaatattga | tatgcccagc | ctgataattt | |
| 5041 | ttaaaaaaaa | aaaa | | | | | |

Sequence NP_995582

| | | | | | | | |
|---|---|---|---|---|---|---|---|
| 1 | mssnsdtgdl | qeslkhgltp | igaglpdrhg | spipargrlv | mlpkveteal | glarshgeqg | (SEQ ID NO:_) |
| 61 | qmpenmqvsq | fkmvnysyde | dleelcpvcg | dkvsgyhygl | ltcesckgff | krtvqnnkry | |
| 121 | tcienqncqi | dktqrkrcpy | crfqkclsvg | mkleavradr | mrggrnkfgp | mykrdralkq | |
| 181 | qkkalirang | lkleamsqvi | qampsdltis | saiqnihsas | kglplnhaal | pptdydrspf | |
| 241 | vtspismtmp | phgslqgyqt | yghfpsraik | seypdpytss | pesimgysym | dsyqtsspas | |
| 301 | iphlilellk | cepdepqvqa | kimaylqqeq | anrskhekls | tfglmckmad | qtlfsivewa | |
| 361 | rssiffrelk | vddqmkllqn | cwsellildh | iyrqvvhgke | gsiflvtgqq | vdysiiasqa | |
| 421 | gatlnnlmsh | aqelvaklrs | lqfdqrefvc | lkflvlfsld | vknlenfqlv | egvqeqvnaa | |
| 481 | lldytmcnyp | qqtekfgqll | lrlpeirais | mqaeeylyyk | hlngdvpynn | lliemlhakr | |
| 541 | a | | | | | | |

Sequence NM_030676

| | | | | | | | |
|---|---|---|---|---|---|---|---|
| 1 | tgttttttcc | ccctttttct | taactttcac | taaggaaatg | agggttactg | tagtctgagg | (SEQ ID NO:_) |
| 61 | tttccttccc | aaagtcacaa | aatatgacaa | gctgcaatct | ttctcacatt | caatgatttc | |
| 121 | tgctgtaagc | caaaggactg | ccaataattt | cgctaagaat | gtctgctagt | ttggatactg | |
| 181 | gagattttca | agaatttctt | aagcatggac | ttacagctat | tgcgtctgca | ccagggtcag | |
| 241 | agactcgcca | ctcccccaaa | cgtgaggaac | aactccggga | aaaacgtgct | gggcttccgg | |
| 301 | accgacaccg | acgccccatt | cccgcccgca | gccgccttgt | catgctgccc | aaagtggaga | |
| 361 | cggaagcccc | aggactggtc | cgatcgcatg | gggaacaggg | gcagatgcca | gaaaacatgc | |
| 421 | aagtgtctca | atttaaaatg | gtgaattact | cctatgatga | agatctggaa | gagctatgtc | |
| 481 | ctgtgtgtgg | cgataaagtg | tctgggtacc | attacggtct | cctcacgtgc | gaaagctgca | |
| 541 | agggtttttt | taagcgaact | gtccaaaacc | aaaaaaggta | cacgtgcata | gagaaccaga | |
| 601 | attgccaaat | tgacaaaacg | cagagaaaac | gatgtcccta | ctgtcgattc | aaaaaatgta | |
| 661 | tcgatgttgg | gatgaagctg | gaagccgtaa | gagccgaccg | catgcgaggg | ggcagaaata | |
| 721 | agtttgggcc | aatgtacaag | agagacaggg | ctttgaagca | gcagaagaaa | gccctcattc | |
| 781 | gagccaatgg | acttaagctg | gaagccatgt | ctcaggtgat | ccaagcaatg | ccctcagacc | |
| 841 | tgacctctgc | aattcagaac | attcattccg | cctccaaagg | cctacctctg | agccatgtag | |
| 901 | ccttgcctcc | gacagactat | gacagaagtc | cctttgtcac | atctcccatt | agcatgacaa | |
| 961 | tgccacctca | cagcagcctg | catggttacc | aaccctatgg | tcactttcct | agtcgggcca | |
| 1021 | tcaagtctga | gtacccagac | ccctactcca | gctcacctga | gtcaatgatg | ggttactcct | |
| 1081 | acatggatgg | ttaccagaca | aactccccgg | ccagcatccc | acacctgata | ctggaacttt | |
| 1141 | tgaagtgtga | accagatgag | cctcaagttc | aagcgaagat | catggcttac | ctccagcaag | |
| 1201 | agcagagtaa | ccgaaacagg | caagaaaagc | tgagcgcatt | tgggctttta | tgcaaaatgg | |
| 1261 | cggaccagac | cctgttctcc | attgttgagt | gggccaggag | tagtatcttc | ttcagggaac | |
| 1321 | tgaaggttga | tgaccaaatg | aagctgcttc | aaaactgctg | gagtgagctc | ttgattctcg | |
| 1381 | atcacattta | ccgacaagtg | gcgcatggga | aggaagggac | aatcttcctg | gttactggag | |
| 1441 | aacacgtgga | ctactccacc | atcatctcac | acacagaagt | cgcgttcaac | aacctcctga | |
| 1501 | gtctcgcaca | ggagctggtg | gtgaggctcc | gttcccttca | gttcgatcag | cgggagtttg | |
| 1561 | tatgtctcaa | gttcctggtg | ctgttcagct | cagatgtgaa | gaacctggag | aacctgcagc | |
| 1621 | tggtggaagg | tgtccaagag | caggtgaatg | ccgccctgct | ggactacacg | gtttgcaact | |
| 1681 | acccacaaca | gactgagaaa | ttcggacagc | tacttcttcg | gctacccgag | atccgggcaa | |
| 1741 | tcagcaagca | ggcagaagac | tacctgtact | ataagcacgt | gaacggggat | gtgccctata | |
| 1801 | ataacctcct | cattgagatg | ctgcatgcca | aaagagccta | agtccccacc | cctggaagct | |
| 1861 | tgctctagga | acacagactg | gaaggagaag | aggaggacga | tgacagaaac | acaatactct | |
| 1921 | gaactgctcc | aagcaatgct | aattataaac | ttggtttaaa | gacactgaat | tttaaaagca | |
| 1981 | taataattaa | atacctaata | gcaaataaat | gatatatcag | ggtatttgta | ctgcaaactg | |
| 2041 | tgaatcaaag | gctgtatgaa | tcaaaggatt | catatgaaag | acattgtaat | ggggtggatt | |
| 2101 | gaacttacag | atggagacca | ataccacagc | agaataaaaa | tggacagaac | aatccttgta | |
| 2161 | tatttaaact | aatctgctat | taagaaattc | agaagttgat | ctctgttatt | aattggattt | |
| 2221 | gtcctgaatt | actccgtggt | gacgctgaac | aactcaagaa | tacatgggct | gtgcttggca | |
| 2281 | gcccctcccc | atccctccca | acaccaccac | ccccaccccc | acaaggccct | ataccttctg | |
| 2341 | acctgtgagc | cctgaagcta | ttttaaggac | ttctgttcag | ccatacccag | tagtagctcc | |
| 2401 | actaaaccat | gatttctgga | tgtctgtgtc | ttagacctgc | caacagctaa | taagaacaat | |
| 2461 | gtataaatat | gtcagcttgc | attttaaata | tgtgctgaag | tttgttttgt | cgtgtgttcg | |
| 2521 | taattaaaaa | gaaaacgggc | agtaaccctc | ttctatataa | gcattagtta | atattaaggg | |
| 2581 | aaatcaaaca | aatctaagcc | aatactccca | acaagcaagt | tagatcttac | ttctgctgct | |
| 2641 | gttgctgaaa | tgtggctttg | gcatggttgg | gtttcataaa | actttttggc | caagaggctt | |
| 2701 | gttagtatac | atccatctgt | ttagtcatca | aggtttgtag | ttcacttaaa | aaaaaataaa | |
| 2761 | ccactagaca | tcttttgctg | aatgtcaaat | agtcacagtc | taagtagcca | aaaagtcaaa | |
| 2821 | gcgtgttaaa | cattgccaaa | tgaaggaaag | ggtgagctgc | aaaggggatg | gttcgaggtt | |
| 2881 | cattccagtt | gtgacccgag | cgtccccaaa | acctgggatg | caaagacagt | gattctgcat | |
| 2941 | atggcctgga | aagacaggaa | agccagtctc | ctacaaaggg | gaatggaaga | tcctggcctc | |
| 3001 | taagtcatag | accaaagtct | gctgtag | | | | |

What is claimed is:

1. A method for identifying compounds that bind to the ligand binding domain of SF-1 or LRH-1, comprising:
contacting a SF-1 or LRH-1 ligand binding domain polypeptide with a test compound; and
determining whether said test compound binds to said SF-1 or LRH-1 ligand binding domain polypeptide, thereby identifying test compounds that bind to the ligand binding domain of SF-1 or LRH-1.

2. The method of claim 1, further comprising determining whether said compound binds in a ligand binding pocket.

3. The method of claim 1, further comprising determining whether said compound binds to a co-activator binding surface.

4. The method of claim 1, further comprising determining whether said compound modulates SF-1 or LRH-1.

5. A method for designing a ligand that binds to SF-1 or LRH-1, comprising:
identifying as one or more molecular scaffolds one or more compounds that bind to a binding site of SF-1 or LRH-1 ligand binding domain polypeptide with low affinity;
determining the orientation of the one or more molecular scaffolds at the binding site of the polypeptide by obtaining co-crystal structures of the one or more molecular scaffolds in the binding site; and
modifying one or more structures of at least one scaffold molecule so as to provide a ligand having altered binding affinity or binding specificity or both for binding to the polypeptide as compared to the binding of the scaffold molecule.

6. The method of claim 5, further comprising synthesizing said ligand.

7. The method of claim 5, wherein said one or more molecular scaffolds interact with at least 3 conserved amino acid residues in a binding pocket of said ligand binding domain.

8. The method of claim 5, wherein said one or more molecular scaffolds interact with at least 3 residues with which a phospholipid ligand interacts.

9. A method for identifying interaction properties of a SF-1 or LRH-1 binding compound, comprising:
identifying at least one conserved interacting amino acid residue in SF-1 or LRH-1 that interacts with said SF-1 or LRH-1 binding compound and at least one other SF-1 or LRH-1 binding compound; and
identifying at least one common interaction property of said binding compound with said conserved residues.

10. The method of claim 9, wherein said interaction property includes an interaction selected from the group consisting of hydrophobic interaction, charge-charge interaction, hydrogen bonding, charge-polar interaction, and polar-polar interaction.

11. A method for developing altered modulators for SF-1 or LRH-1, comprising:
selecting a molecular scaffold from a set of at least 3 molecular scaffolds that bind to SF-1 or LRH-1; and
modifying one or more structures of said scaffold molecule so as to provide a ligand having altered binding affinity or binding specificity or both for binding to the SF-1 or LRH-1 as compared to the binding of said molecular scaffold.

12. A method of identifying a modulator of a SF-1 or LRH-1 ligand binding domain polypeptide, comprising: designing or selecting a compound that interacts with amino acid residues in a ligand binding site of said SF-1 or LRH-1 ligand binding domain polypeptide, based upon a crystal structure of said ligand binding domain polypeptide, so as to provide said modulator.

13. The method of claim 12, wherein said crystal structure is a structure of SF-1 or LRH-1 ligand binding domain in complex with one or more of a ligand and a coactivator polypeptide.

14. The method of claim 12, further comprising synthesizing said modulator.

15. The method of claim 12, further comprising determining whether said compound modulates the activity of the SF-1 or LRH-1 polypeptide.

16. The method of claim 12, wherein said amino acid residues are conserved residues.

17. The method of claim 12, wherein said amino acid residues interact with a phospholipid ligand.

18. A method for designing a modulator that modulates the activity of a SF-1 or LRH-1, comprising:
evaluating the three-dimensional structure of crystallized SF-1 or LRH-1 ligand binding domain polypeptide complexed with one or more of a ligand and a coactivator polypeptide; and
synthesizing or selecting a compound based on the three-dimensional structure of said crystal complex that will bind to the SF-1 or LRH-1 ligand binding domain polypeptide.

19. The method of claim 18, further comprising determining whether said compound modulates the activity of SF-1 or LRH-1.

20. A protein crystal, comprising substantially pure SF-1 ligand binding domain polypeptide.

21. The crystal of claim 20, further comprising a ligand.

22. The crystal of claim 21, wherein said ligand is a phospholipid ligand.

23. A protein crystal, comprising substantially pure LRH-1 ligand binding domain polypeptide.

24. The crystal of claim 23, further comprising a ligand.

25. The crystal of claim 24, wherein said ligand is a phospholipid ligand.

26. A method for determining the three-dimensional structure of a crystallized SF-1 or LRH-1 ligand binding domain polypeptide in complex with one or more of a ligand and a coactivator polypeptide, comprising:
crystallizing substantially pure SF-1 or LRH-1 ligand binding domain polypeptide in complex with one or more of a ligand and a coactivator polypeptide to form a crystallized complex; and
analyzing the crystallized complex to determine the three-dimensional structure of the SF-1 or LRH-1 ligand binding domain polypeptide in complex with one or more of a ligand and a coactivator polypeptide.

27. The method of claim 26, wherein said ligand is a phospholipid ligand.

28. A method of treating a SF-1 or LRH-1 mediated disease or condition in a mammal, comprising: administering to said mammal a therapeutically effective amount of a SF-1 or LRH-1 modulator designed according to the method of claim 5, a prodrug of such modulator, or a pharmaceutically acceptable salt of such modulator or prodrug.

29. The method of claim 28, wherein said disease or condition is elevated cholesterol.

30. The method of claim 28, wherein said disease or condition is cancer.

31. The method of claim 28, wherein said disease or condition is hepatitis B virus infection.

32. The method of claim 28, wherein said disease or condition is a developmental defect or risk therof.

33. A method for identifying structurally and energetically allowed sites on a binding compound for attachment of an additional component, comprising: analyzing the orientation of the binding compound in a SF-1 or LRH-1 binding site, thereby identifying accessible sites on the compound for attachment of the additional component.

34. The method of claim 33, further comprising calculating the change in binding energy on attachment of the additional component at one or more of the accessible sites.

35. The method of claim 33, wherein the orientation is determined by co-crystallography.

36. The method of claim 33, wherein said additional component includes a linker.

37. The method of claim 33, wherein said additional component includes a label.

38. The method of claim 33, wherein said additional component includes a solid phase material.

39. A method for attaching a SF-1 or LRH-1 binding compound to an attachment component without substantially altering the ability of said SF-1 or LRH-1 binding compound to bind SF-1 or LRH-1, comprising:
identifying energetically allowed sites for attachment of said attachment component on the binding compound; and
attaching the binding compound or derivative thereof to the attachment component at the energetically allowed site.

40. A method for making an affinity matrix for SF-1 or LRH-1, comprising:
identifying energetically allowed sites on a SF-1 or LRH-1 binding compound for attachment to a solid phase matrix without substantially altering the ability of said SF-1 or LRH-1 binding compound to bind SF-1 or LRH-1; and
attaching said binding compound to said solid phase matrix through the energetically allowed site.

41. A modified SF-1 ligand binding domain, comprising a SF-1 ligand binding domain polypeptide modified by subsitution of surface cysteines, C247 or C412 or both.

42. The modified SF-1 ligand binding of claim 41 domain wherein said substitutions are substitution by serine residues.
